(19) United States
(12) Patent Application Publication
(10) Pub. No.: US 2020/0016159 A1
(43) Pub. Date: Jan. 16, 2020

(54) N3-CYCLICALLY SUBSTITUTED THIENOURACILS AND USE THEREOF

(71) Applicants: Bayer Aktiengesellschaft, Leverkusen (DE); Bayer Pharma Aktiengesellschaft, Berlin (DE)

(72) Inventors: Michael HAERTER, Leverkusen (DE); Dirk KOSEMUND, Berlin (DE); Yolanda CANCHO GRANDE, Leverkusen (DE); Martina DELBECK, Heiligenhaus (DE); Bernd KALTHOF, Wuppertal (DE); Klemens LUSTIG, Wuppertal (DE); Frank SUESSMEIER, Muenchen (DE)

(21) Appl. No.: 16/335,282

(22) PCT Filed: Sep. 18, 2017
(86) PCT No.: PCT/EP2017/073504

§ 371 Date: Mar. 21, 2019

(30) Foreign Application Priority Data
Sep. 23, 2016 (EP) 16190351.3
Jun. 30, 2017 (EP) 17179105.6

(51) Int. Cl. A61K 31/519; A61K 9/20; A61K 9/00; A61K 9/08; C07D 495/04

(52) U.S. Cl. CPC A61K 31/519; A61K 9/2027; C07D 495/04; A61K 9/08; A61K 9/0053

(57) ABSTRACT

The present application relates to novel thieno[2,3-d]pyrimidine-2,4-dione (“thienouracil”) derivatives having cyclic substituents in the 3 position, to processes for the preparation thereof, to the use thereof alone or in combinations for treatment and/or prevention of diseases and to the use thereof for production of medicaments for treatment and/or prevention of diseases, especially for treatment and/or prevention of pulmonary and cardiovascular disorders and of cancer.

The present application relates to novel thieno[2,3-d]pyrimidine-2,4-dione (“thienouracil”) derivatives having cyclic substituents in the 3 position, to processes for the preparation thereof, to the use thereof alone or in combinations for treatment and/or prevention of diseases and to the use thereof for production of medicaments for treatment and/or prevention of diseases, especially for treatment and/or prevention of pulmonary and cardiovascular disorders and of cancer.

The endogenous purine nucleoside adenosine is formed ubiquitously and modulates, as important signal molecule, a large number of physiological and pathophysiological processes. Most of it is formed during the intra- and extracellular degradation of adenine nucleotides, and a smaller amount is formed during the intracellular hydrolysis of S-adenosyl homocysteine. Under physiological conditions, extracellular adenosine can be re-phosphorylated by adenosine kinase to adenosine monophosphate (AMP) or rearranged by adenosine deaminase to inosine. The extracellular concentration is between 30 and 300 nM. As a result of tissue damage caused, for example, by hypoxia, in inflammation reaction and during oxidative stress, there is an increased formation and accumulation of adenosine, such that the extracellular concentration may increase to up to 15 μM.

The biological actions of adenosine are mediated via G-protein-coupled receptors located at the plasma membrane. Currently, four adenosine receptor subtypes have been demonstrated: A1 adenosine receptor (A1R), A2a adenosine receptor (A2aR), A2b adenosine receptor (A2bR) and A3 adenosine receptor (A3R). From among the adenosine receptors mentioned above, the A2b receptor has the weakest affinity for adenosine. For this reason, in contrast to the other adenosine receptors, it is not activated under normal physiological conditions. A1 and A3 receptors are coupled to Gi proteins which inhibit adenylate cyclase, whereas A2a and A2b receptors, via Gs proteins, stimulate adenylate cyclase, thus causing an intracellular increase of cAMP. Via Gq proteins, both the A1, the A3 and the A2b receptor activate phospholipase C which cleaves membrane-bound phosphatidylinositol-4,5-bisphosphate into inositol-1,4,5-triphosphate and diacylglycerol. This in turn leads to an increase of the intracellular calcium concentration and activation of further target proteins such as protein kinase C and the MAP kinases.

A2b receptors are expressed on pulmonary epithelial and smooth muscle cells, vascular endothelial and smooth muscle cells, fibroblasts and also inflammatory cells. Expression of the A2b receptor at the cell surface is a dynamic process and is greatly enhanced, for example, by hypoxia, inflammatory factors and free radicals. The adenosine-activated A2b receptors lead to formation and release of pro-inflammatory and pro-fibrotic cytokines such as, for example, IL-6, IL-4 and IL-8. Studies have shown that the A2b receptor plays an important role at the chronic stage of pulmonary disorders during tissue remodelling and promotes inter alia differentiation of fibroblasts in myofibroblasts, resulting in enhanced synthesis and deposition of collagen. In pulmonary tissue samples of patients suffering from idiopathic pulmonary fibrosis, COPD and pulmonary hypertension associated with COPD [Zhou et al., PLoS One 5, e9224 (2010); Selmann et al., PLoS One 2, e482 (2007)] and in various animal models of fibro-proliferative pulmonary disorders [Karmouty-Quintana et al., Am. J. Respir. Cell Mol. Biol. 49 (6), 1038-1047 (2013); Karmouty-Quintana et al., FASEB J. 26, 2546-2557 (2012); Sun et al., J. Clin. Invest. 116, 2173-2182 (2006)], it was possible to detect an increased expression of the A2b receptor. In the animal model of bleomycin-induced pulmonary fibrosis and pulmonary hypertension in the mouse, a genetic knock-out of the A2b receptor resulted both in inhibition of the progression of pulmonary fibrosis and pulmonary vascular remodeling and the resulting pulmonary hypertension [Karmouty-Quintana et al., Faseb J. 26, 2546-2557 (2012)]. It is assumed that the release of inter alia endothelin-1 (ET-1) and interleukin-6 (IL-6) from vascular cells, which is modulated by the A2b receptor, plays a role during the development of pulmonary hypertension associated with pulmonary fibrosis. Stimulation of human pulmonary arterial endothelial and smooth muscle cells with 5′-(N-ethylcarboxamido)adenosine (NECA), an adenosine analogue, results in the release of ET-1 and IL-6, which can be prevented by A2b receptor inhibition [Karmouty-Quintana et al., Faseb J. 26, 2546-2557 (2012)]. Elevated endothelin-1- and IL-6 concentrations were found in lung tissue and serum of patients suffering from pulmonary hypertension [Giaid et al., N. Engl. J. Med. 329, 1967-1968 (1993); Steiner et al., Circ. Res. 104, 236-244 (2009)]. Furthermore, it is assumed that the A2b receptor-mediated release of inter alia IL-6 and other profibrotic mediators and stimulation of the differentiation of fibroblasts in myofibroblasts in the lung leads to induction of fibrosis. Stimulation of human fibroblasts with NECA leads to the release of IL-6 which is increased by hypoxia and can be prevented by inhibiting the A2b receptor. It was possible to demonstrate an increased IL-6 expression in patients suffering from idiopathic pulmonary fibrosis and in animal models of pulmonary fibrosis [Zhong et al., Am. J. Respir. Cell Mol. Biol. 32, 2-8 (2005); Cavarra et al., Am. J. Physiol. Lung Cell. Mol. Physiol. 287, L1186-L1192 (2004)].

The A2b receptor also plays an important role in tissue remodelling after myocardial infarction. In the animal model of the permanent ligature of the coronary artery in the mouse, inhibition of the A2b receptor resulted in a reduction of caspase-1 activity and the invasion of inflammatory cells in heart tissue and the cytokines and adhesion molecules in plasma and in an improvement of systolic and diastolic heart function [Toldo et al., J. Pharmacol. Exp. Ther. 343, 587-595 (2012)].

In tumours and surrounding tissue, the local adenosine concentration is frequently greatly elevated as a result of the occurrence of hypoxia, as a result of necrotic processes or else because of genetic and epigenetic changes in tumour cells which lead to elevated extracellular production of adenosine with simultaneously reduced degradation and reduced cellular uptake of adenosine [J. Blay et al., Cancer Res. 57 (13), 2602-2605 (1997); G. Schulte, B. B. Fredholm, Cell Signal. 15 (9), 813-827 (2003)]. This leads to activation of the above-described adenosine receptors in tumour cells, tumour-associated cells and cells in the tissue surrounding the tumour. The signalling chains initiated as a result trigger various kinds of processes, the majority of which promote tumour growth and the spread thereof to other sites in the organism. For that reason, the inhibition of the adenosine signalling pathways constitutes a valuable strategy for treatment of cancer. For example, the inhibition of the A2b receptor-mediated adenosine signalling pathway with the A2b receptor antagonist MRS1754 leads to reduced growth of colon cancer cell lines [D.-F. Ma et al., Hum. Pathol. 41 (11), 1550-1557 (2010)]. The A2b receptor antagonist PSB603 reduces the growth of several prostate cancer cell lines [Q. Wei et al., Purinergic Signal. 9 (2), 271-280 (2013)].

The influence of adenosine on tumour metastases appears to be greater than the direct influence on the proliferation of tumour cells. This involves A2b receptor-mediated adenosine signalling chains in particular, and the blockage of the A2b receptor—both genetically and pharmacologically with A2b receptor antagonists—leads to reduced migration of tumour cells in vitro and reduced formation of metastases in animal models [J. Stagg et al., Proc. Natl. Acad. Sci. USA 107 (4), 1547-1552 (2010); C. J. Desmet et al., Proc. Natl. Acad. Sci. USA 110 (13), 5139-5144 (2013); E. Ntantie et al., Sci. Signal. 6 (277), ra39 (2013)].

Adenosine also affects the tumour-associated vascular endothelium: A2b receptor-mediated adenosine signalling chains lead to release of pro-angiogenic factors from various human tumour cell lines, but also from tumour-associated immune cells, and thus stimulate neovascularization, which promotes tumour growth [S. Ryzhov et al., Neoplasia 10 (9), 987-995 (2008); S. Merighi et al., Mol. Pharmacol. 72 (2), 395-406 (2007); S. Merighi et al., Neoplasia 11 (10), 1064-1073 (2009)].

There is increasingly better understanding of the importance of the immune system in suppression of tumour development, tumour growth and metastasis. It is found in this context that adenosine is capable of reducing the immune reaction [S. Gessi et al., Biochim. Biophys. Acta Biomembranes 1808 (5), 1400-1412 (2011); J. Stagg et al., Proc. Natl. Acad. Sci. USA 107 (4), 1547-1552 (2010); D. Jin et al., Cancer Res. 70 (6), 2245-2255 (2010); S. F. M. Häusler et al., Cancer Immunol. Immunother. 60 (10), 1405-1418 (2011); J. Spychala, Pharmacol. Ther. 87 (2-3), 161-173 (2000)]. The inhibition of the A2b receptor-mediated adenosine signalling pathway with the A2b receptor antagonist PSB603, in contrast, leads to a reduction in tumour growth and metastasis in melanoma animal models, which is attributed to inhibition of tumour-induced suppression of the immune system [W. Kaji et al., J. Toxicol. Sci. 39 (2), 191-198 (2014)]. This improvement is caused by a reduction in the proportion of regulatory T cells, which reduce the immune response, in the overall immune cell infiltrate in the presence of the A2b receptor antagonist. At the same time, the populations of cytotoxic CD8+ T cells and CD4+ T helper cells are increased. Furthermore, immunosuppressant effects of adenosine on further cells in the immune system have been described (M1 and M2 macrophages, dendritic cells, myeloid suppressor cells), some of which are mediated by the A2b receptor [B. Csoka et al., FASEB J. 26 (1), 376-386 (2012); S. V. Novitskiy et al., Blood 112 (5), 1822-1831 (2008); M. Yang et al., Immunol. Cell Biol. 88 (2), 165-171 (2010); S. Ryzhov et al., J. Immunol. 187 (11), 6120-6129 (2011)]. In animal models of bladder tumours and breast tumours, the A2b receptor antagonist ATL801 brings about slowing of tumour growth and a distinct reduction in metastasis [C. Cekic et al., J. Immunol. 188 (1), 198-205 (2012)]. These effects are accompanied by an ATL801-induced increase in the number of tumour antigen-presenting dendritic cells and a significant increase in the interferon γ level and, as a result, elevated concentrations of chemokine CXCL10, which in turn leads to activation of CXCR3+ T cells and ultimately to improved immune defence against tumour growth and metastasis.

It is therefore assumed that the A2b receptor plays an important role in many disorders, injuries and pathological changes whose aetiology and/or progression is associated with inflammatory events and/or proliferative and fibro-proliferative tissue and vessel remodelling. These may especially be disorders of and/or damage to the lung, the cardiovascular system or the kidney, or the disorder may be a blood disorder, a neoplastic disease or another inflammatory disorder.

Disorders of and damage to the lung which may be mentioned in this context are in particular idiopathic pulmonary fibrosis, pulmonary hypertension, bronchiolitis obliterans syndrome (BOS), chronic-obstructive pulmonary disease (COPD), asthma and cystic fibrosis. Disorders of and damage to the cardiovascular system in which the A2b receptor is involved are, for example, tissue changes following myocardial infarction and associated with heart failure. Renal disorders are, for example, renal insufficiency and kidney failure. An example of a blood disorder is sickle cell anemia. Examples of tissue degradation and remodeling in the event of neoplastic processes are the invasion of cancer cells into healthy tissue (formation of metastases) and neovascularization (neoangiogenesis). Another inflammatory disease where the A2b receptor is involved is, for example, multiple sclerosis.

Idiopathic fibrosis of the lung or idiopathic pulmonary fibrosis (IPF) is a progressive lung disease which, left untreated, results in death within an average of 2.5 to 3.5 years after diagnosis. At the time of diagnosis, patients are usually more than 60 years old, men being slightly more frequently affected than women. Onset of IPF is insidious and characterized by increasing shortness of breath and a dry tickly cough. IPF is one of the group of idiopathic interstitial pneumonias (IIP), a heterogeneous group of pulmonary disorders which are characterized by fibrosis and inflammation of varying severity which can be distinguished using clinical, imaging and fine tissue criteria. Within this group, idiopathic pulmonary fibrosis is of particular significance owing to its frequency and aggressive progression [Ley et al., Am. J. Respir. Crit. Care Med. 183, 431-440 (2011)]. IPF may either occur sporadically or be hereditary. As yet, the causes are unknown. However, in recent years there have been numerous indications that chronic damage of the alveolar epithelium leads to the release of profibrotic cytokines/mediators followed by increased fibroblast proliferation and increased collagen fiber formation, resulting in a patchy fibrosis and the typical honeycomb structure of the lung [Strieter et al., Chest 136, 1364-1370 (2009)]. The clinical sequelae of fibrotization are a decrease in the elasticity of the pulmonary tissue, a reduced diffusing capacity and the development of severe hypoxia. With regard to lung function, a corresponding worsening of the forced vital capacity (FVC) and the diffusing capacity (DLCO) can be detected. Essential and prognostically important comorbidities of IPF are acute exacerbation and pulmonary hypertension [Beck et al., Pneumologe 10, 105-111 (2013)]. The prevalence of pulmonary hypertension in interstitial pulmonary disorders is 10-40% [Lettieri et al., Chest 129, 746-752 (2006); Behr et al., Eur. Respir. J. 31, 1357-1367 (2008)]. Currently, there is no curative treatment for IPF—except for lung transplantation.

Pulmonary hypertension (PH) is a progressive lung disease which, left untreated, results in death within an average of 2.8 years after diagnosis. By definition, the mean pulmonary arterial pressure (mPAP) in case of chronic pulmonary hypertension is >25 mmHg at rest or >30 mmHg under exertion (normal value <20 mmHg). The pathophysiology of pulmonary hypertension is characterized by vasoconstriction and remodeling of the pulmonary vessels. In chronic PH, there is a neomuscularization of primarily unmuscularized lung vessels, and the circumference of the vascular musculature of the vessels already muscularized increases. This increasing obliteration of the pulmonary circulation results in progressive stress on the right heart, which leads to a reduced output from the right heart and eventually ends in right heart failure [M. Humbert et al., J. Am. Coll. Cardiol. 2004, 43, 13S-24S]. Idiopathic (or primary) pulmonary arterial hypertension (IPAH) is a very rare disorder, whereas secondary pulmonary hypertension (non-PAH PH, NPAHPH) is very common, and it is thought that the latter is currently the third most common group of cardiovascular disorders after coronary heart disease and systemic hypertension [Naeije, in: A. J. Peacock et al. (Eds.), Pulmonary Circulation. Diseases and their treatment, 3^{rd} edition, Hodder Arnold Publ., 2011, p. 3]. Since 2008, pulmonary hypertension is classified in accordance with the Dana Point classification into various sub-groups according to the respective etiology [D. Montana and G. Simonneau, in: A. J. Peacock et al. (Eds.), Pulmonary Circulation. Diseases and their treatment, 3^{rd} edition, Hodder Arnold Publ., 2011, pp. 197-206].

Despite all the advances in the therapy of PH there is as yet no prospect of cure of this serious disorder. Standard therapies available on the market (for example prostacyclin analogs, endothelin receptor antagonists, phosphodiesterase inhibitors) are able to improve the quality of life, the exercise tolerance and the prognosis of the patients. These are therapeutic principles which are administered systemically and act primarily hemodynamically by modulating vessel tone. The applicability of these medicaments is limited owing to side effects, some of which are serious, and/or complicated administration forms. The period over which the clinical situation of the patients can be improved or stabilized by specific monotherapy is limited (for example owing to the development of tolerance). Eventually the therapy escalates and thus a combination therapy is applied, where a plurality of medicaments must be given concurrently. Currently, these standard therapeutics are approved only for the treatment of pulmonary arterial hypertension (PAH). In the case of secondary forms of PH such as PH-COPD, these therapeutic principles (for example sildenafil, bosentan) fail in clinical studies since, as a result of non-selective vasodilation, they lead to a reduction (desaturation) of the arterial oxygen content in the patients. The probable reason for this is an unfavorable effect on the ventilation-perfusion adaptation in the lung in heterogeneous lung disorders owing to the systemic administration of non-selective vasodilators [I. Blanco et al., Am. J. Respir. Crit. Care Med. 2010, 181, 270-278; D. Stolz et al., Eur. Respir. J. 2008, 32, 619-628].

Novel combination therapies are one of the most promising future therapeutic options for the treatment of pulmonary hypertension. In this connection, the finding of novel pharmacological mechanisms for the treatment of PH is of particular interest [Ghofrani et al., Herz 2005, 30, 296-302; E. B. Rosenzweig, Expert Opin. Emerging Drugs 2006, 11, 609-619; T. Ito et al., Curr. Med. Chem. 2007, 14, 719-733]. In particular, such novel therapeutic approaches which can be combined with the therapy concepts already on the market may form the basis of a more efficient treatment and thus be of great advantage for the patients.

In the context of the present invention, the term “pulmonary hypertension” includes both primary and secondary sub-forms (NPAHPH) as defined according to the Dana Point classification in accordance with their respective etiology [D. Montana and G. Simonneau, in: A. J. Peacock et al. (Eds.), Pulmonary Circulation. Diseases and their treatment, 3^{rd} edition, Hodder Arnold Publ., 2011, p. 197-206; Hoeper et al., J. Am. Cardiol., 2009, 54 (1), Suppl. S, S85-S96]. These include in particular in group 1 pulmonary arterial hypertension (PAH), which, among others, embraces the idiopathic and the familial forms (IPAH and FPAH, respectively). Furthermore, PAH also embraces persistent pulmonary hypertension of the newborn and the associated pulmonary arterial hypertension (APAH) associated with collagenoses, congenital systemic pulmonary shunt lesions, portal hypertension, HIV infections, the intake of certain drugs and medicaments (for example of appetite suppressants), with disorders having a significant venous/capillary component such as pulmonary venoocclusive disorder and pulmonary capillary hemangiomatosis, or with other disorders such as disorders of the thyroid, glycogen storage diseases, Gaucher disease, hereditary teleangiectasia, hemoglobinopathies, myeloproliferative disorders and splenectomy. Group 2 of the Dana Point classification comprises PH patients having a causative left heart disorder, such as ventricular, atrial or valvular disorders. Group 3 comprises forms of pulmonary hypertension associated with a lung disorder, for example with chronic obstructive lung disease (COPD), interstitial lung disease (ILD), pulmonary fibrosis (IPF), and/or hypoxemia (e.g. sleep apnea syndrome, alveolar hypoventilation, chronic high-altitude sickness, hereditary deformities). Group 4 includes PH patients having chronic thrombotic and/or embolic disorders, for example in the case of thromboembolic obstruction of proximal and distal pulmonary arteries (CTEPH) or non-thrombotic embolisms (e.g. as a result of tumor disorders, parasites, foreign bodies). Less common forms of pulmonary hypertension, such as in patients suffering from sarcoidosis, histiocytosis X or lymphangiomatosis, are summarized in group 5.

Bronchiolitis obliterans syndrome (BOS) is a chronic rejection reaction after a lung transplant. Within the first five years after a lung transplant about 50-60% of all patients are affected, and within the first nine years more than 90% of patients [Estenne et al., Am. J. Respir. Crit. Care Med. 166, 440-444 (2003)]. The cause of the disease has not been elucidated. In spite of numerous improvements in the treatment of transplantation patients, the number of BOS cases has hardly changed over the last years. BOS is the most important long-term complication in lung transplantations and is considered to be the main reason for the fact that survival rates are still markedly below those for other organ transplantations. BOS is an inflammatory event which is associated with changes in the lung tissue affecting primarily the small respiratory passages. Damage and inflammatory changes of the epithelial cells and the subepithelial structures of the smaller respiratory passages lead, owing to ineffective regeneration of the epithelium and aberrant tissue repair, to excessive fibroproliferation. There is scarring and finally destruction of the bronchi and also clots of granulation tissue in the small respiratory passages and alveolae, occasionally with vascular involvement. The diagnosis is based on the lung function. In BOS, there is a worsening of the FEV1 compared to the average of the two best values measured postoperatively. Currently, there is no curative treatment of BOS. Some of the patients show improvements under intensified immunosuppression; patients not showing any response experience persistent deterioration, such that retransplantation is indicated.

Chronic obstructive pulmonary disease (COPD) is a slowly progressing pulmonary disease characterized by an obstruction of respiratory flow which is caused by pulmonary emphysema and/or chronic bronchitis. The first symptoms of the disease generally manifest themselves during the fourth or fifth decade of life. In the subsequent years of life, shortness of breath frequently becomes worse, and there are instances of coughing combined with copious and purulent sputum, and stenotic respiration extending as far as breathlessness (dyspnea). COPD is primarily a smokers' disease: smoking is the cause of 90% of all cases of COPD and of 80-90% of all COPD-related deaths. COPD is a big medical problem and constitutes the sixth most frequent cause of death worldwide. Of people over the age of 45, about 4-6% are affected. Although the obstruction of the respiratory flow may only be partial and temporal, COPD cannot be cured. Accordingly, the aim of treatment is to improve the quality of life, to alleviate the symptoms, to prevent acute worsening and to slow the progressive impairment of lung function. Existing pharmacotherapies, which have hardly changed over the last two or three decades, are the use of bronchodilators to open blocked respiratory passages, and in certain situations corticosteroids to control the inflammation of the lung [P. J. Barnes, N. Engl. J. Med. 343, 269-280 (2000)]. The chronic inflammation of the lung, caused by cigarette smoke or other irritants, is the driving force of the development of the disease. The basic mechanism comprises immune cells which, during the inflammatory reaction of the lung, release proteases and various cytokines which cause pulmonary emphysema and remodeling of the bronchi.

It is therefore an object of the present invention to provide novel substances which act as potent and selective antagonists of the adenosine A2b receptor and are suitable as such for treatment and/or prevention in particular of pulmonary and cardiovascular disorders and of cancer.

WO 2009/037468-A1 discloses 2-aminothieno[3,2-d]pyrimidine-4-carboxamides as adenosine A2b antagonists for treatment of asthma, COPD, diabetes and cancer. Antagonists of the adenosine A2a receptor that are especially suitable for treatment of CNS and addiction disorders are 6-heteroaryl-substituted thieno[2,3-d]pyrimidine-2,4-diones described in WO 2007/103776-A2, and 6-styryl-substituted thieno[2,3-d]pyrimidine-2,4-diones described in WO 2008/070529-A2. WO 98/54190-A1, WO 00/12514-A1, GB 2 363 377-A and US 2004/0122028-A1 disclose various thieno[2,3-d]pyrimidine-2,4-diones which can be used, inter alia, for treatment of inflammatory and proliferative disorders. U.S. Pat. No. 6,140,325 discloses carboxylate-substituted thieno[2,3-d]pyrimidine-2,4-diones as endothelin receptor antagonists. WO 00/61583-A1 claims xanthine analogues suitable for treatment of inflammatory, neurodegenerative and autoimmune disorders. WO 02/064598-A1 and WO 2004/014916-A1 describe bicyclic pyrimidinediones as inhibitors of matrix metalloproteinases (MMPs), especially of MMP-13. WO 2013/071169-A1, WO 2014/182943-A1 and WO 2014/182950-A1 disclose thieno[2,3-d]pyrimidine-2,4-diones as ACC inhibitors for treatment of infections and metabolic disorders. WO 2015/052065-A1 recently disclosed cyclic thienouracil-6-carboxamides as adenosine A2b receptor antagonists for treatment of disorders of the lung and the cardiovascular system, and WO 2016/023832-A1 discloses 3-(hydroxyalkyl)-substituted thieno[2,3-d]pyrimidin-2,4-diones as TRPC5 modulators for treatment of neurological disorders. In the intervening period, WO 2016/150901-A1 has published various 6-(heterocyclylmethyl)-substituted thienouracils as adenosine A2b receptor antagonists, and WO 2017/075056-A1 discloses further thieno[2,3-d]pyrimidine-2,4-dione derivatives as ACC inhibitors for treatment of infections and metabolic disorders.

The present invention provides compounds of the general formula (I)

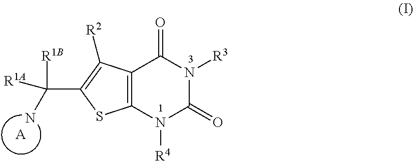

in which
the ring A is an azaheterocycle of the formula

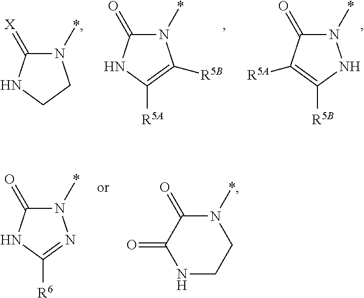

in which * marks the bond to the adjoining C(R^{1A})(R^{1B}) group,
R^{5A} and R^{5B} are the same or different and are independently hydrogen or (C₁-C₄)-alkyl,
R⁶ is hydrogen or (C₁-C₄)-alkyl,
and
X is O, S or N(R′) in which
   R⁷ represents cyano, methoxycarbonyl or ethoxycarbonyl,
R^{1A} and R^{1B} are independently hydrogen or deuterium,
R² is methyl or ethyl,
R³ is cyclopropyl, cyclobutyl, cyclopentyl, spiro[3.3]hept-2-yl, 3-oxetanyl or 3-tetrahydrofuranyl,
   where cyclopropyl, cyclobutyl, cyclopentyl and spiro[3.3]hept-2-yl may be up to disubstituted identically or differently by a radical selected from fluorine, methyl, ethyl, trifluoromethyl and methoxy,
   and
   where 3-oxetanyl and 3-tetrahydrofuranyl may be up to disubstituted identically or differently by a radical selected from fluorine and methyl,
and
R⁴ is methyl, ethyl, 2-fluoroethyl, 2,2-difluoroethyl, 2,2,2-trifluoroethyl, n-propyl, 3-cyanopropyl, 3-fluoropropyl, 3,3-difluoropropyl, 3,3,3-trifluoropropyl, n-butyl, 4-fluorobutyl, 4,4,4-trifluorobutyl, 3,3,4,4-tetrafluorobutyl, n-pentyl, iso-pentyl or n-hexyl,
or
R⁴ is a group of the formula —CH₂—R⁸ in which
   R⁸ is cyano, cyclopropyl, cyclobutyl, cyclopentyl, 2-oxetanyl, 3-oxetanyl, 2-tetrahydrofuranyl or 3-tetrahydrofuranyl,
   where cyclopropyl, cyclobutyl and cyclopentyl may be up to disubstituted by fluorine,
or
R⁴ is a group of the formula —CH₂—CH₂—OR⁹ or —CH₂—CH₂—SR¹⁰, in which
   R⁹ is methyl, trifluoromethyl, ethyl or iso-propyl
   and
   R^{9A} is methyl or trifluoromethyl,
      and solvates thereof.

Compounds of the invention are the compounds of the formula (I) and solvates thereof, the compounds of the formulae (I-1), (I-1a), (I-1b), (I-1c), (I-1d), (I-1e), (1-2), (I-3), (I-4), (I-5), (I-6), (I-7) and (I-8) adduced hereinafter that are encompassed by the formula (I) and solvates thereof, and the compounds that are encompassed by formula (I) and are described hereinafter as working examples and solvates thereof, if the compounds adduced hereinafter are not already solvates.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers; E/Z double bond isomers). The present invention therefore encompasses the enantiomers, diastereomers and double bond isomers, and the respective mixtures thereof. The stereoisomerically homogeneous constituents can be isolated from such mixtures in a known manner; chromatography processes are preferably used for this, in particular HPLC chromatography on an achiral or chiral phase.

If the compounds of the invention can occur in tautomeric forms, the present invention encompasses all the tautomeric forms.

In the context of the present invention, unless specified otherwise, the substituents and radicals are defined as follows:

In the context of the invention, (C₁-C₄)-alkyl is a straight-chain or branched alkyl radical having 1 to 4 carbon atoms. Preferred examples include: methyl, ethyl, n-propyl, isopropyl, n-butyl, isobutyl, sec-butyl and tert-butyl.

In the context of the present invention, all radicals which occur more than once are defined independently of one another. When radicals in the compounds of the invention are substituted, the radicals may be mono- or polysubstituted, unless specified otherwise. Substitution by one substituent or by two identical or different substituents is preferred. Particular preference is given to substitution by one substituent.

A specific embodiment of the present invention comprises compounds of the formula (I) in which the ring A is an azaheterocycle of the formula

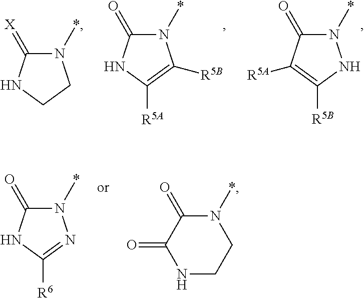

in which * marks the bond to the adjoining C(R^{1A})(R^{1B}) group,
R^{5A} and R^{5B} are the same or different and are independently hydrogen or (C₁-C₄)-alkyl,
R⁶ is hydrogen or (C₁-C₄)-alkyl,
and
X is O, S or N(R′) in which
   R⁷ represents cyano, methoxycarbonyl or ethoxycarbonyl,
R^{1A} and R^{1B} are independently hydrogen or deuterium,
R² is methyl or ethyl,
R³ is cyclopropyl, cyclobutyl, cyclopentyl, spiro[3.3]hept-2-yl, 3-oxetanyl or 3-tetrahydrofuranyl,
   where cyclopropyl, cyclobutyl, cyclopentyl and spiro[3.3]hept-2-yl may be up to disubstituted identically or differently by a radical selected from fluorine, methyl, ethyl, trifluoromethyl and methoxy,
   and
   where 3-oxetanyl and 3-tetrahydrofuranyl may be up to disubstituted identically or differently by a radical selected from fluorine and methyl,
and
R⁴ is methyl, ethyl, n-propyl, 3-fluoropropyl, 3,3-difluoropropyl, 3,3,3-trifluoropropyl, n-butyl, n-pentyl, iso-pentyl or n-hexyl,
or
R⁴ is a group of the formula —CH₂—R⁸ in which
   R⁸ is cyano, cyclopropyl, cyclobutyl, cyclopentyl, 2-oxetanyl, 3-oxetanyl, 2-tetrahydrofuranyl or 3-tetrahydrofuranyl,
      where cyclopropyl, cyclobutyl and cyclopentyl may be up to disubstituted by fluorine,
or
R⁴ is a group of the formula —CH₂—CH₂—OR⁹ in which
   R⁹ is methyl, trifluoromethyl, ethyl or iso-propyl,
      and solvates thereof.

Preference is given in the context of the present invention to compounds of the formula (I) in which the ring A is an azaheterocycle of the formula

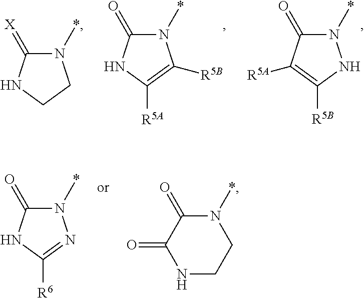

in which * marks the bond to the adjoining C(R^{1A})(R^{1B}) group,
R^{5A} and R^{5B} are the same or different and are independently hydrogen or methyl,
R⁶ is hydrogen or methyl,
and
X is O or N(R⁷) in which
   R⁷ represents cyano or methoxycarbonyl,
R^{1A} and R^{1B} are both hydrogen or both deuterium,
R² is methyl,
R³ is cyclopropyl, cyclobutyl, cyclopentyl or spiro[3.3]hept-2-yl,
   where cyclopropyl, cyclobutyl, cyclopentyl and spiro[3.3]hept-2-yl may be up to disubstituted identically or differently by a radical selected from fluorine, methyl and methoxy,
and
R⁴ is methyl, ethyl, 2-fluoroethyl, 2,2-difluoroethyl, 2,2,2-trifluoroethyl, n-propyl, 3-fluoropropyl, 3,3-difluoropropyl, 3,3,3-trifluoropropyl, n-butyl, 4,4,4-trifluorobutyl, n-pentyl or n-hexyl,
or
R⁴ is a group of the formula —CH₂—R⁸ in which
   R⁸ is cyclopropyl, cyclobutyl or 2-tetrahydrofuranyl,
      where cyclopropyl and cyclobutyl may be up to disubstituted by fluorine,
or
R⁴ is a group of the formula —CH₂—CH₂—OR⁹ in which
   R⁹ is methyl or trifluoromethyl,
      and solvates thereof.

A further preferred embodiment of the present invention encompasses compounds of the formula (I) in which

the ring A is an azaheterocycle of the formula

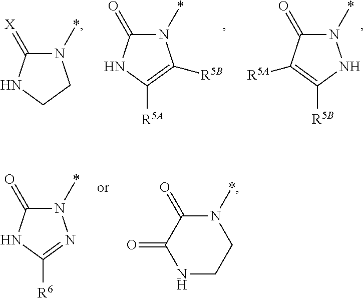

in which * marks the bond to the adjoining C(R^{1A})(R^{1B}) group,
R^{5A} and R^{5B} are the same or different and are independently hydrogen or methyl,
R⁶ is hydrogen or methyl,
and
X is O or N(R⁷) in which
   R⁷ represents cyano or methoxycarbonyl,
R^{1A} and R^{1B} are both hydrogen or both deuterium,
R² is methyl,
R³ is cyclopropyl, cyclobutyl, cyclopentyl or spiro[3.3]hept-2-yl,
   where cyclopropyl, cyclobutyl, cyclopentyl and spiro[3.3]hept-2-yl may be up to disubstituted identically or differently by a radical selected from fluorine, methyl and methoxy,
and
R⁴ is n-propyl, 3-fluoropropyl, 3,3-difluoropropyl, 3,3,3-trifluoropropyl, n-butyl, n-pentyl or n-hexyl,
or
R⁴ is a group of the formula —CH₂—R⁸ in which
   R⁸ is cyclopropyl, cyclobutyl or 2-tetrahydrofuranyl,
or
R⁴ is a group of the formula —CH₂—CH₂—OR⁹ in which
   R⁹ is methyl or trifluoromethyl,
      and solvates thereof.

A particular embodiment of the present invention relates to compounds of the formula (I) in which the ring A is an azaheterocycle of the formula

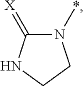

in which * marks the bond to the adjoining C(R^{1A})(R^{1B}) group
and
X is O or N(R⁷⁾ in which
   R⁷ represents cyano or methoxycarbonyl,
      and solvates thereof.

A further particular embodiment of the present invention relates to compounds of the formula (I) in which

the ring A is an azaheterocycle of the formula

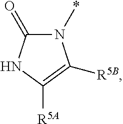

in which * marks the bond to the adjoining C(R^{1A})(R^{1B}) group and
R^{5A} and R^{5B} are each hydrogen,
   and solvates thereof.

A further particular embodiment of the present invention relates to compounds of the formula (I) in which

the ring A is an azaheterocycle of the formula

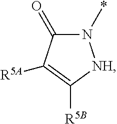

in which * marks the bond to the adjoining C(R^{1A})(R^{1B}) group
and
R^{5A} and R^{5B} are each hydrogen,
   and solvates thereof.

A further particular embodiment of the present invention relates to compounds of the formula (I) in which

the ring A is an azaheterocycle of the formula

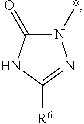

in which * marks the bond to the adjoining C(R^{1A})(R^{1B}) group
and
R⁶ is hydrogen,
   and solvates thereof.

A further particular embodiment of the present invention relates to compounds of the formula (I) in which

the ring A is an azaheterocycle of the formula

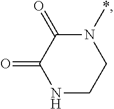

in which * marks the bond to the adjoining C(R^{1A})(R^{1B}) group,
   and solvates thereof.

A further particular embodiment of the present invention relates to compounds of the formula (I) in which

R^{1A} and R^{1B} are both hydrogen,
and solvates thereof.

A further particular embodiment of the present invention relates to compounds of the formula (I) in which

R² is methyl,
and solvates thereof.

A further particular embodiment of the present invention relates to compounds of the formula (I) in which

R³ is cyclopropyl, cyclobutyl or cyclopentyl,
where cyclopropyl, cyclobutyl and cyclopentyl may be up to disubstituted, identically or differently, by a radical selected from fluorine and methyl,
   and solvates thereof.

A further particular embodiment of the present invention relates to compounds of the formula (I) in which

R⁴ is 3-fluoropropyl, 3,3,3-trifluoropropyl or n-butyl,
and solvates thereof.

A further particular embodiment of the present invention relates to compounds of the formula (I) in which

R⁴ is methyl, ethyl, 2-fluoroethyl, 2,2-difluoroethyl, 2,2,2-trifluoroethyl or n-propyl, and solvates thereof.

A further particular embodiment of the present invention relates to compounds of the formula (I) in which

R⁴ is a group of the formula —CH₂—R⁸ in which
R⁸ is cyclopropyl or cyclobutyl,
   where cyclopropyl and cyclobutyl may be up to disubstituted by fluorine,
      and solvates thereof.

A further particular embodiment of the present invention relates to compounds of the formula (I) in which

R⁴ is a group of the formula —CH₂—CH₂—OR⁹ in which
R⁹ is methyl or trifluoromethyl,
   and solvates thereof.

In the context of the present invention, particular preference is given to compounds of the formula (I) in which

the ring A is an azaheterocycle of the formula

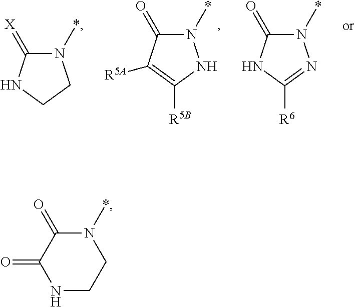

in which * marks the bond to the adjoining C(R^{1A})(R^{1B}) group,
R^{5A} and R^{5B} are each hydrogen,
R⁶ is hydrogen,
and
X is O or N(R⁷) in which
   R⁷ represents cyano or methoxycarbonyl,
R^{1A} and R^{1B} are both hydrogen or both deuterium,
R² is methyl,
R³ is cyclopropyl, cyclobutyl or cyclopentyl,
   where cyclopropyl, cyclobutyl and cyclopentyl may be up to disubstituted, identically or differently, by a radical selected from fluorine and methyl,
      and
      R⁴ is methyl, ethyl, 2-fluoroethyl, 2,2-difluoroethyl, 2,2,2-trifluoroethyl, n-propyl, 3-fluoropropyl, 3,3,3-trifluoropropyl or n-butyl,
      or
      R⁴ is a group of the formula —CH₂—R⁸ in which
   R⁸ is cyclopropyl or cyclobutyl,
      where cyclopropyl and cyclobutyl may be up to disubstituted by fluorine,
         or
         R⁴ is a group of the formula —CH₂—CH₂—OR⁹ in which
   R⁹ is methyl or trifluoromethyl,
      and solvates thereof.

A further particularly preferred embodiment of the present invention encompasses compounds of the formula (I) in which

the ring A is an azaheterocycle of the formula

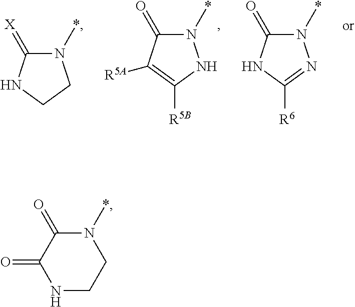

n which * marks the bond to the adjoining C(R^{1A})(R^{1B}) group,
R^{5A} and R^{5B} are each hydrogen,
R⁶ is hydrogen,
and
X is O or N(R⁷) in which
   R⁷ represents cyano or methoxycarbonyl,
R^{1A} and R^{1B} are both hydrogen or both deuterium,
R² is methyl,
R³ is cyclopropyl, cyclobutyl or cyclopentyl,
   where cyclopropyl, cyclobutyl and cyclopentyl may be up to disubstituted, identically or differently, by a radical selected from fluorine and methyl,
and
R⁴ is 3-fluoropropyl, 3,3,3-trifluoropropyl or n-butyl,
or
R⁴ is a group of the formula —CH₂—R⁸ in which
   R⁸ is cyclopropyl or cyclobutyl,
or
R⁴ is a group of the formula —CH₂—CH₂—OR⁹ in which
   R⁹ is methyl or trifluoromethyl,
      and solvates thereof.

The individual radical definitions specified in the respective combinations or preferred combinations of radicals are, independently of the respective combinations of the radicals specified, also replaced as desired by radical definitions of other combinations.

Very particular preference is given to combinations of two or more of the abovementioned preferred ranges.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to the comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of the invention may therefore possibly also constitute a preferred embodiment of the present invention. Isotopic variants of the compounds of the invention can be prepared by commonly used processes known to those skilled in the art, for example by the methods described further down and the procedures described in the working examples, by using corresponding isotopic modifications of the respective reagents and/or starting compounds.

Furthermore, the present invention also encompasses prodrugs of the compounds of the invention. The term “prodrugs” refers here to compounds which may themselves be biologically active or inactive, but are converted while present in the body, for example by a metabolic or hydrolytic route, to compounds of the invention.

The inventive compounds of the formula (I) may, depending on the respective nature of the azaheterocycle A, be prepared by different routes, some of which are also alternative routes.

For instance, inventive compounds of the formula (I-1)

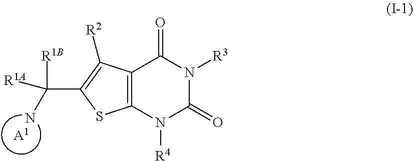

in which
the ring A¹ is an azaheterocycle of the formula

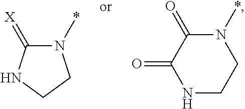

in which * marks the bond to the adjacent C(R^{1A})(R^{1B}) group and X has the definition given above,
R^{1A} and R^{1B} are both hydrogen,
and
R², R³ and R⁴ are as defined above,
can be prepared by a general method according to the following Reaction Scheme 1:

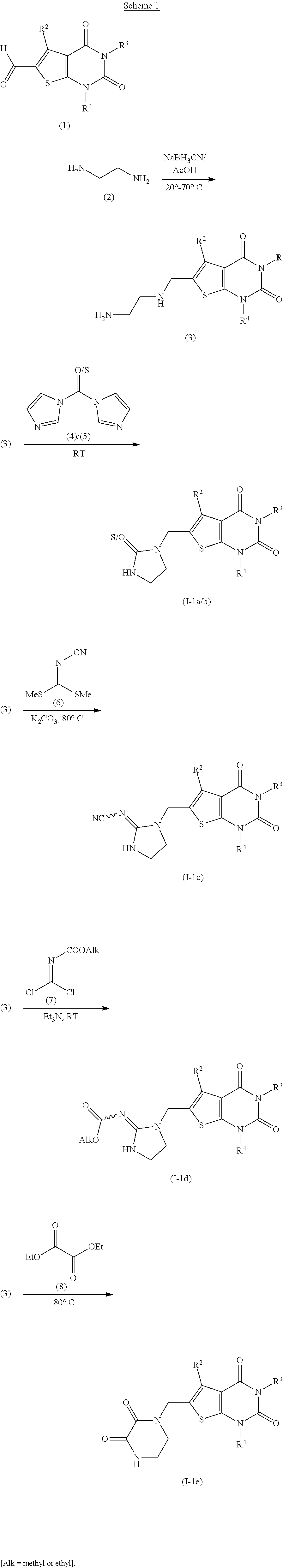

Thienouracil carbaldehydes of the formula (1) are first reacted with 1,2-diaminoethane (2) in a reductive amination to give the diamino compounds of the formula (3). A suitable reducing agent is especially sodium cyanoborohydride or sodium borohydride, each in the presence of acetic acid. A suitable solvent is methanol or ethanol, optionally in a mixture with dichloromethane, and the reaction is preferably effected within a temperature range between RT and +70° C. With regard to yield and simplicity of product isolation, it can be advantageous in this reaction, rather than the free diamine (2), to use a carbamate-protected derivative, for example tert-butyl (2-aminoethyl)carbamate or benzyl (2-aminoethyl)carbamate, and then to detach the protecting group (tert-butoxycarbonyl or benzyloxycarbonyl) again by customary methods in the resulting amination product which is analogous to (3).

The target compounds of the formulae (I-1a) and (I-1b) are obtained by subsequent reaction of the diamino compounds (3) with N,N′-carbonyldiimidazole (4) [for (I-1a)] or N,N′-thiocarbonyldiimidazole (5) [for (I-1b)]. The reactions are preferably effected at RT and in solvents such as tetrahydrofuran (THF), 1,4-dioxane or dimethyl sulfoxide (DMSO), optionally in the presence of a tertiary amine base, for example triethylamine. The products of the formula (I-1c) are obtained by reaction of the diamino compounds (3) with dimethyl N-cyanodithioiminocarbonate (6). The reaction is preferably effected in N,N-dimethylformamide (DMF) as solvent in the presence of alkali metal carbonates, for example potassium carbonate, as base at elevated temperatures around +80° C. The products of the formula (I-1d) are obtained by reaction of the diamino compounds (3) with methyl or ethyl (dichloromethylene)carbamate (7). The reaction is preferably effected in dichloromethane as solvent in the presence of a tertiary amine base, for example triethylamine, at RT. Finally, the products of the formula (I-1e) are obtained by reaction of the diamino compounds (3) with diethyl oxalate (8). The reaction is preferably effected in ethanol as solvent at elevated temperatures around +80° C.

Alternatively, inventive compounds of the formula (1-2)

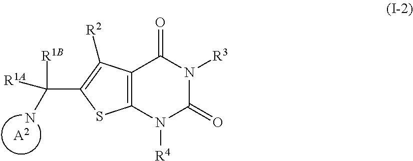

in which
the ring A² is an azaheterocycle of the formula

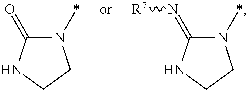

in which * marks the bond to the adjacent C(R^{1A})(R^{1B}) group and R⁷ has the definition
   given above,
   R^{1A} and R^{1B} are both hydrogen or both deuterium,
   and
   R², R³ and R⁴ are as defined above,
   can be prepared by a general method according to the following Reaction Scheme 2:

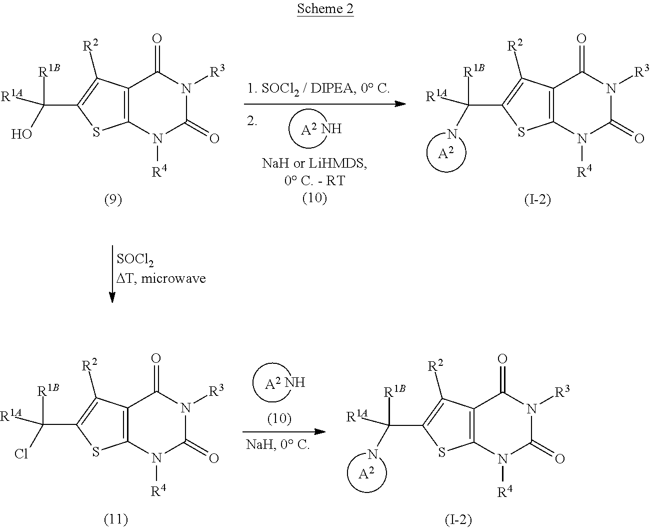

In the “one-pot” variant of this process, an alcohol of the formula (9) is converted first with a chlorinating agent, such as preferably thionyl chloride, in the presence of a tertiary amine base, for example N,N-diisopropylethylamine or triethylamine, to the corresponding chloro compound [corresponding to formula (11)]. This chloro compound is not isolated but admixed in the same reaction vessel with a solution of the deprotonated azaheterocycle of the formula (10), in order thus to obtain the target compound of the formula (1-2) in one step. Suitable bases for the deprotonation of the heterocycle (10) are strong bases, for example alkali metal hydrides or alkali metal amides; preference is given to using sodium hydride or lithium hexamethyldisilazide. The chlorination step is typically effected in a halogenated hydrocarbon as inert solvent—preference being given here to dichloromethane—in the temperature range around 0° C. The solution of the deprotonated heterocycle (10) is added at the same temperature. The substitution reaction to give (1-2) is then preferably effected at RT. Suitable solvents for preparation of the deprotonated heterocycle (10) are especially N,N-dimethylformamide (DMF), tetrahydrofuran (THF) or mixtures thereof. The deprotonation itself is preferably effected within a temperature range between 0° C. and +60° C.

Less hydrolysis-sensitive chloro compounds of the formula (11) can be prepared and also isolated by—similarly to the manner above—reacting alcohols of the formula (9) with a chlorinating agent, such as preferably thionyl chloride, in an inert solvent, for example chloroform or dichloromethane. The reaction is effected here preferably within a temperature range between RT and +80° C., and it has been found to be particularly advantageous for the heating above the boiling point of the particular solvent to use a microwave oven with employment of closed reaction vessels. In a subsequent, separate reaction step, the isolated chloro compounds of the formula (11) are then reacted under similar conditions, as elucidated above, with a solution of the deprotonated heterocycle (10).

In the above-described process, the azaheterocycles of the formula (10) in question can also be used in protected form with use of a suitable amide protecting group that masks one of the two NH groups, if necessary or required for avoidance of side reactions. Amide protecting groups of this kind are familiar to those skilled in the art (with regard to the suitability, introduction and removal of amide protecting groups see, for example, T. W. Greene and P. G. M. Wuts, Protective Groups in Organic Synthesis, Wiley, New York, 1999].

In a further, alternative process, inventive compounds of the formula (1-3)

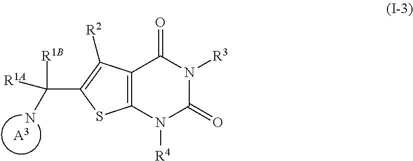

in which
the ring A³ is a cyclic urea derivative of the formula

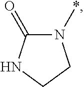

in which * marks the bond to the adjoining C(R^{A})(R^{1B}) group,
   R^{1A} and R^{1B} are both hydrogen,
   and
   R², R³ and R⁴ are as defined above,
   can be prepared according to the following Reaction Scheme 3:

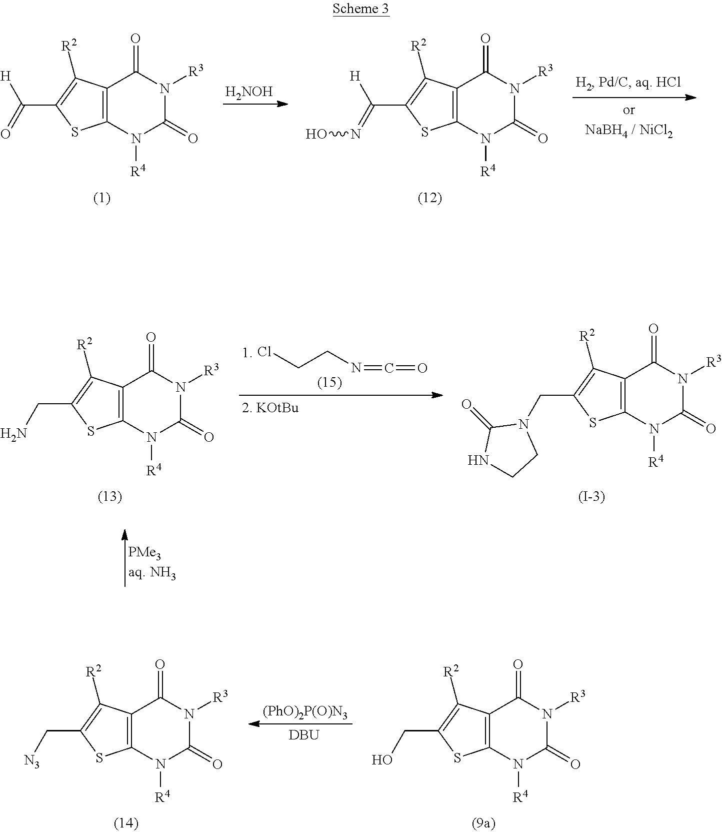

Here, aldehydes of the formula (1) are first converted with hydroxylamine to the corresponding oximes of the formula (12). The reaction is preferably effected at RT using an aqueous hydroxylamine solution in a water-miscible ether such as tetrahydrofuran (THF) as solvent. The subsequent reduction to give the aminomethyl compounds (13) can be achieved by hydrogenation in the presence of a noble metal catalyst. Preferred reaction conditions are hydrogen pressure 1 bar at RT in the presence of a catalytic amount of palladium (5-10% on charcoal) in methanol or ethanol as solvent. Preferably, the hydrogenation is effected in the presence of aqueous mineral acid, for example concentrated hydrochloric acid. Alternatively, the reduction to the aminomethyl compounds (13) can also be effected with sodium borohydride in the presence of suitable metal salts, for example nickel chloride or cobalt chloride. Preferred reaction conditions here are the use of sodium borohydride in combination with nickel(II) chloride hexahydrate in methanol as solvent at RT. Another route to the aminomethyl compounds of the formula (13) proceeds from the alcohols of the formula (9a). These are first converted to the corresponding azides of the formula (14) by reacting them with diphenylphosphoryl azide in the presence of an amine base, for example 1,8-diazabicyclo[5.4.0]undec-7-ene (DBU), at 0° C. to RT in tetrahydrofuran (THF). The reduction of azides (14) to the aminomethyl compounds (13) can then be effected, for example, by reacting with trimethylphosphine in tetrahydrofuran (THF) and concentrated aqueous ammonia at RT.

The aminomethyl compounds of the formula (13) obtained by one of the routes mentioned are then reacted in a one-pot process with chloroethyl isocyanate (15), at first forming an open-chain urea derivative. The reaction is preferably effected at RT in a solvent mixture of N,N-dimethylformamide (DMF) and tetrahydrofuran (THF), or in toluene within the temperature range between +60° C. and the boiling point of the solvent. The subsequent addition of a strong base, for example potassium tert-butoxide, to the reaction mixture at RT then results in the ring closure to give the target compounds of the formula (1-3).

Inventive compounds of the formula (1-4)

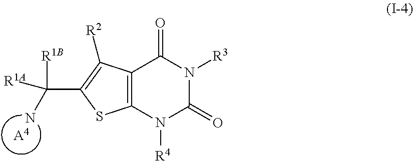

in which
the ring A⁴ is a 1,3-dihydroimidazol-2-one derivative of the formula

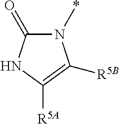

in which * marks the bond to the adjacent C(R^{1A})(R^{1B}) group and R^{5A} and R^{5B} have the definitions given above,
   R^{1A} and RIB are both hydrogen,
   and
   R², R³ and R⁴ are as defined above,
   can be obtained according to the following Reaction Scheme 4:

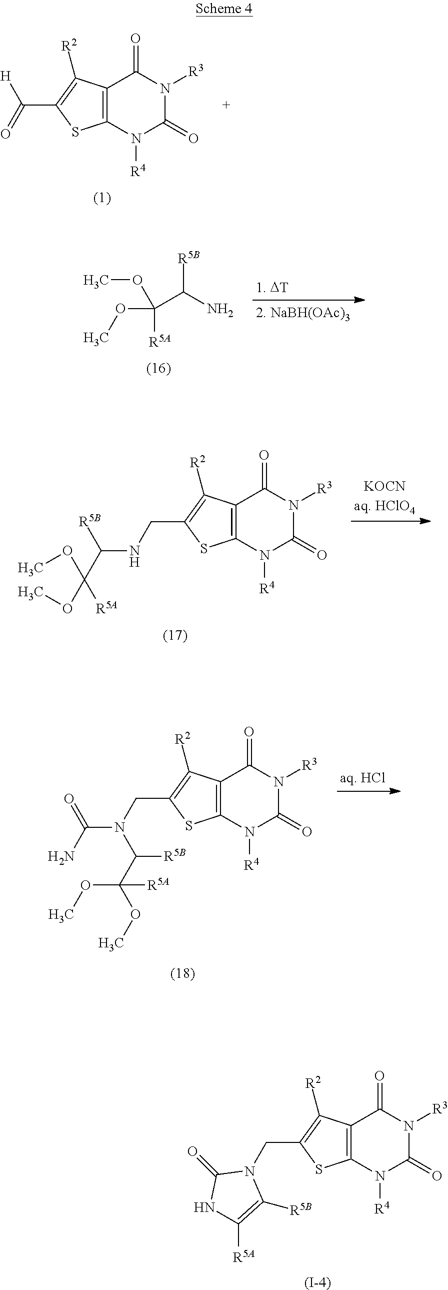

Aldehydes of the formula (1) are first heated to reflux here with amino acetals or amino ketals of the formula (16) in a suitable solvent, such as methanol or dichloromethane, in the manner of a reductive amination and then reduced at RT with sodium triacetoxyborohydride to the compounds of the formula (17). These are subsequently converted with potassium cyanate and aqueous perchloric acid in methanol at RT to the urea derivatives of the formula (18). In the last reaction step, the simultaneous acetal or ketal cleavage and ring closure are effected under acid catalysis to give the target compounds of the formula (1-4). The reaction is effected in methanol at RT with hydrochloric acid of different concentration (from 0.5 mol/l up to concentrated hydrochloric acid).

In formula (16) and the subsequent intermediates (17) and (18), the dimethyl acetal/ketal is shown; however, it is also possible to use other standard acetals or ketals in this process, especially cyclic examples such as 1,3-dioxolane or 1,3-dioxane derivatives.

Inventive compounds of the formula (1-5)

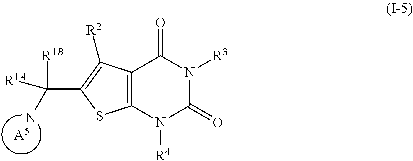

in which
the ring A⁵ is a 2,4-dihydro-1,2,4-triazol-3-one derivative of the formula

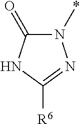

in which * marks the bond to the adjoining C(R^{1A})(R^{1B}) group
and
R⁶ is hydrogen,
   R^{1A} and RIB are both hydrogen,
   and
   R², R³ and R⁴ are as defined above,
   can be prepared by an alternative route according to Reaction Scheme 5:

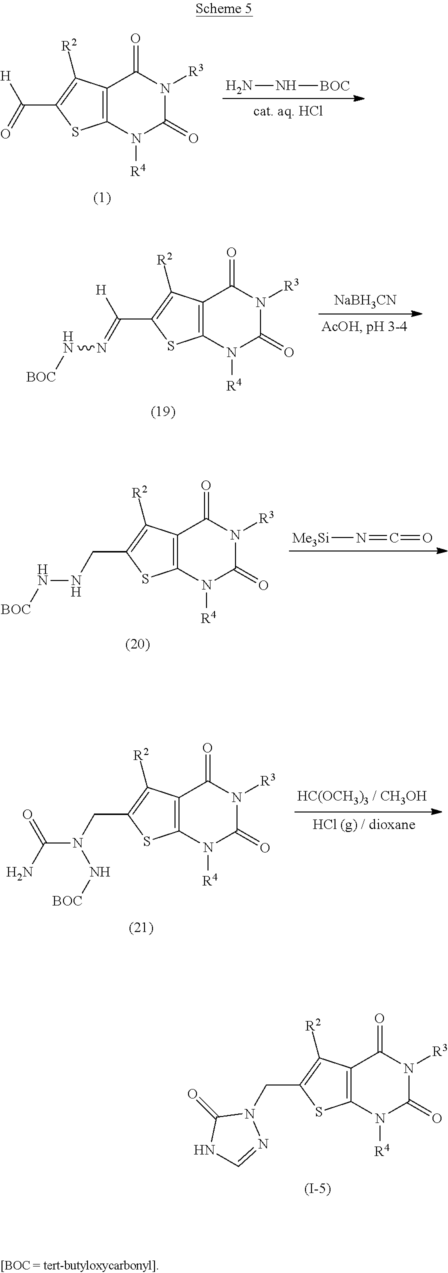

Aldehydes of the formula (1) are converted by reaction with BOC-protected hydrazine in ethanol and in the presence of a catalytic amount of concentrated hydrochloric acid at RT to the hydrazones of the formula (19), which are then converted with sodium cyanoborohydride in methanol at +65° C. to the hydrazine derivatives of the formula (20). The exact control of the pH plays a major role in the latter reaction: in the presence of Bromocresol Green as indicator, addition of acetic acid in portions maintains a pH of about 3-4 over the entire reaction time. The compounds of the formula (20) are then reacted with trimethylsilyl isocyanate to give urea derivatives of the formula (21). The reaction is conducted in an alcohol as solvent, preferably in isopropanol, at elevated temperature, preferably at about 50° C. Under these conditions, there is also simultaneous detachment of the trimethylsilyl group. The ring closure to give the target compounds of the formula (1-5) is achieved by acid-mediated reaction with trimethyl orthoformate. For this purpose, the compounds of the formula (21) are treated in the presence of hydrogen chloride with an excess of trimethyl orthoformate in methanol. This reaction is preferably conducted at room temperature.

Inventive compounds of the formula (1-6)

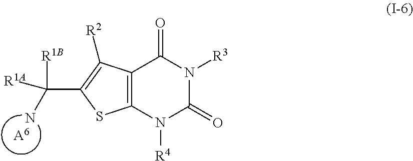

in which
the ring A⁶ is a 2,4-dihydro-1,2,4-triazol-3-one of the formula

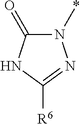

in which * marks the bond to the adjacent C(R^{1A})(R^{1B}) group and R⁶ has the definition given above,
   R^{1A} and R^{1B} are both hydrogen,
   and
   R², R³ and R⁴ are as defined above,
   can be prepared according to the following Reaction Scheme 6:

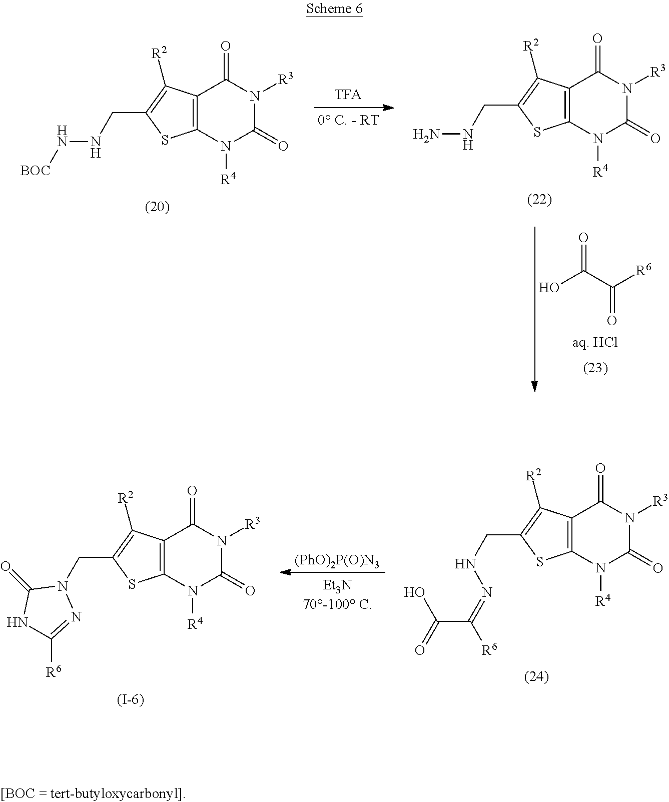

In this process, the protected hydrazine derivative of the formula (20) (see Scheme 5) is converted first with trifluoroacetic acid in dichloromethane to the free hydrazine of the formula (22). The BOC detachment is effected within a temperature range between 0° C. and RT, preferably at 0° C. In order to avoid breakdown of the product, the reaction time chosen should be no longer than required; in addition, subsequent workup and purifying operations should be conducted at no higher than RT. In analogy to a previously described two-stage process [see U.S. Pat. No. 6,077,814, Referential Production Examples 1-4], the hydrazine of the formula (22) is first condensed with glyoxylic acid (23) [R⁶═H] under acid catalysis to give the hydrazone of the formula (24). The reaction is effected in water in the presence of hydrochloric acid within a temperature range between 0° C. and RT, preferably at +10° C. to +20° C. Subsequently, the hydrazonocarboxylic acid (24) is converted with diphenylphosphoryl azide (DPPA) to the corresponding carbonyl azide which then gives the corresponding isocyanate in situ in the manner of a Curtius rearrangement, and then the latter cyclizes spontaneously to give the triazolone derivative of the formula (1-6). The reaction is effected in an inert solvent, for example toluene, and in the presence of a tertiary amine base, for example triethylamine. The reaction is conducted initially within a temperature range between about +40° C. and +80° C.; later on, the reaction temperature is then increased to +100° C. to +110° C.

By using appropriate 2-oxocarboxylic acids (23), it is also possible in principle by this process to obtain those inventive compounds of the formula (1-6) in which R⁶ is (C₁-C₄)-alkyl.

Alternatively, inventive compounds of the formula (1-7)

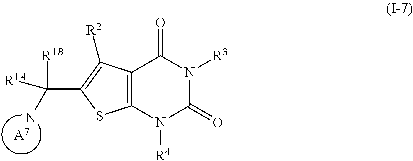

in which
the ring A⁷ is a 2,4-dihydro-1,2,4-triazol-3-one of the formula

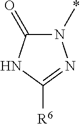

in which * marks the bond to the adjacent C(R^{1A})(R^{B}) group and R⁶ has the definition given above,
   R^{1A} and R^{1B} are both hydrogen or both deuterium,
   and
   R², R³ and R⁴ are as defined above,
   can be prepared according to the following Reaction Scheme 7:

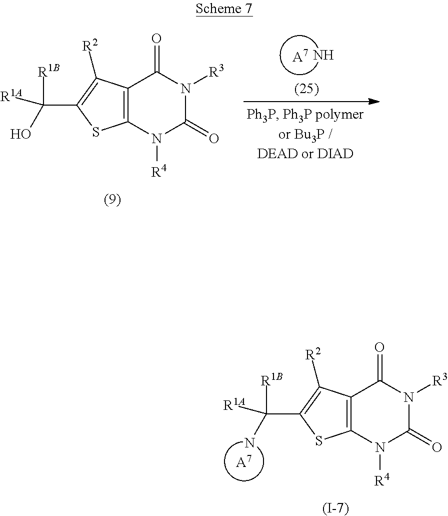

Alcohols of the formula (9) are reacted here in the manner of a Mitsunobu reaction by a direct route with an azaheterocycle of the formula (25) to give the target compounds of the formula (1-7). Suitable reagents for this transformation are, for example, triphenylphosphine, polymer-bound triphenylphosphine, tributylphosphine or trimethylphosphine, each in combination with diethyl azodicarboxylate (DEAD), diisopropyl diazodicarboxylate (DIAD) or azodicarboxylic acid dipiperidide (ADDP) [cf., for example, D. L. Hughes, Org. Reactions 42, 335 (1992); D. L. Hughes, Org. Prep. Proced. Int. 28 (2), 127 (1996)]. The reaction is preferably conducted in tetrahydrofuran (THF) or dichloromethane as solvent within a temperature range between 0° C. and RT.

In this process, the azaheterocycle (25) can also be used in protected form with use of a suitable amide protecting group that masks the N⁴ atom of the 1,2,4-triazol-3-one, if necessary or required for avoidance of side reactions. Amide protecting groups of this kind are familiar to those skilled in the art (with regard to the suitability, introduction and removal of amide protecting groups see, for example, T. W. Greene and P. G. M. Wuts, Protective Groups in Organic Synthesis, Wiley, New York, 1999].

Inventive compounds of the formula (1-8)

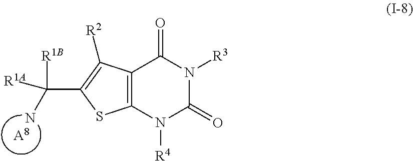

in which
the ring A⁸ is a 1,2-dihydropyrazol-3-one derivative of the formula

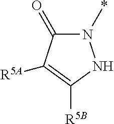

in which * marks the bond to the adjacent C(R^{1A})(R^{1B}) group and R^{5A} and R^{5B} have the definitions given above,
   R^{1A} and R^{1B} are both hydrogen,
   and
   R², R³ and R⁴ are as defined above,
   can be prepared according to the following Reaction Scheme 8:

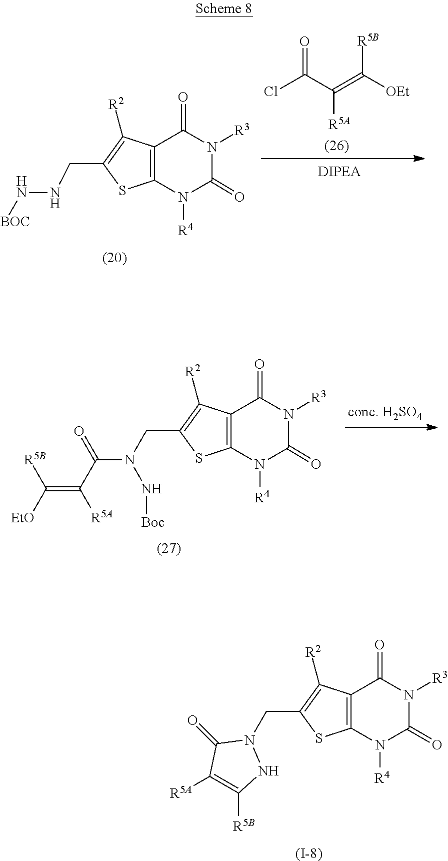

The reaction of the hydrazine derivatives of the formula (20) (see Scheme 5) with the acryloyl chlorides of the formula (26) is conducted under standard conditions, for example in dichloromethane as solvent within a temperature range between 0° C. and RT and in the presence of a tertiary amine base, for example N,N-diisopropylethylamine. The final acid-catalysed removal of the Boc protecting group and the subsequent ring closure to give the target compounds of the formula (I-8) are effected at RT either in pure concentrated sulfuric acid or in dichloromethane with an added catalytic amount of concentrated sulfuric acid.

The synthesis of the thienouracil intermediates of the formulae (1) and (9) used for the preparation of the compounds of the invention [see Schemes 1, 2, 3, 4, 5 and 7] is shown in the following Reaction Schemes 9-13:

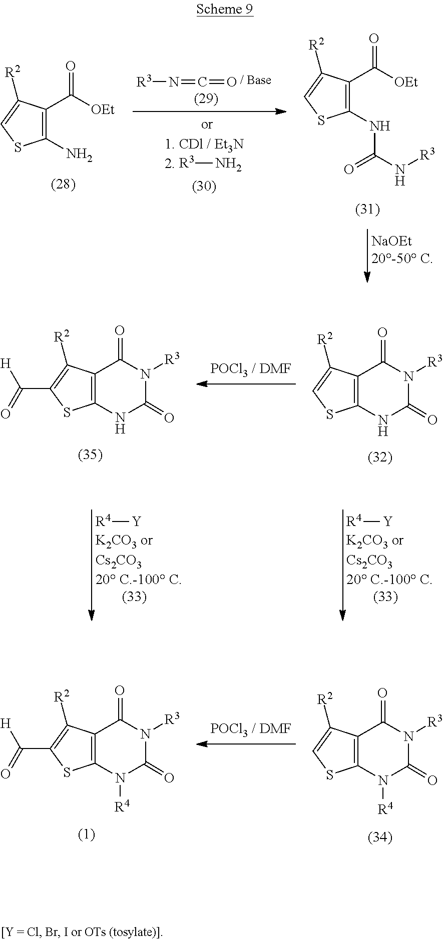

2-Aminothiophene-3-carboxylic esters of the formula (28) are converted here to the ureas of the formula (31) either with isocyanates of the formula (29) or, after activation with N,N′-carbonyldiimidazole (CDI), by reaction with amines of the formula (30). The reaction with the isocyanates (29) is preferably effected in an ethereal solvent, for example in tetrahydrofuran (THF), and in the presence of a tertiary amine base, for example triethylamine, under reflux conditions, or in pyridine as solvent and base at a temperature of about +50° C. The activation of the 2-aminothiophene-3-carboxylic ester (28) with CDI is likewise conducted in the presence of a tertiary amine base, for example triethylamine, in an inert solvent, preferably in tetrahydrofuran (THF) or dichloromethane, at RT and sometimes takes prolonged reaction times of several days. After addition of the amine component (30) to the CDI-activated 2-aminothiophene-3-carboxylic ester, there is generally rapid further reaction at RT to give the ureas of the formula (31). Subsequent treatment with alkali metal alkoxides in the corresponding alcohol as solvent (for example and with preference sodium ethoxide in ethanol) achieves ring closure to give the thienouracils of the formula (32) in a clean reaction. Depending on the substituent R³, the reaction already proceeds at RT, or it requires a somewhat elevated temperature around +50° C.

The subsequent alkylation with the compounds of the formula (33) is conducted in the presence of an inorganic base, for example potassium carbonate or caesium carbonate, in an inert solvent, for example and with preference N,N-dimethylformamide (DMF), tetrahydrofuran (THF), acetonitrile or mixtures thereof. The reaction temperature is typically between RT and about +100° C. In the case of volatile alkylating agents (33), it is found to be helpful to use closed reaction vessels and heating by means of a microwave oven. Depending on the nature of the leaving group Y, it may be advantageous to conduct the alkylation in the presence of a catalytic amount of potassium iodide. The compounds of the formula (34) thus obtained are then converted in a Vilsmeier-Haack reaction with a mixture of phosphorus oxychloride and N,N-dimethylformamide (DMF) in an exothermic reaction to the aldehydes of the formula (1). Typically, the heat released during the reaction is sufficient to achieve full conversion. Sometimes, however, it may also be necessary to heat the mixture to about +90° C. for a while after the heat of reaction has abated.

The above reaction sequence of alkylation and formylation can also be conducted in the reverse sequence, by first converting the thienouracils of the formula (32) under the conditions of the Vilsmeier-Haack reaction already described to the formyl derivatives of the formula (35) and then alkylating the latter under the conditions likewise already described with the compounds of the formula (33) to give the target aldehydes of the formula (1).

Alternatively, the aldehydes of the formula (1) can also be prepared from the thienouracil derivatives (32) or (34) [see Scheme 9] by the following general process:

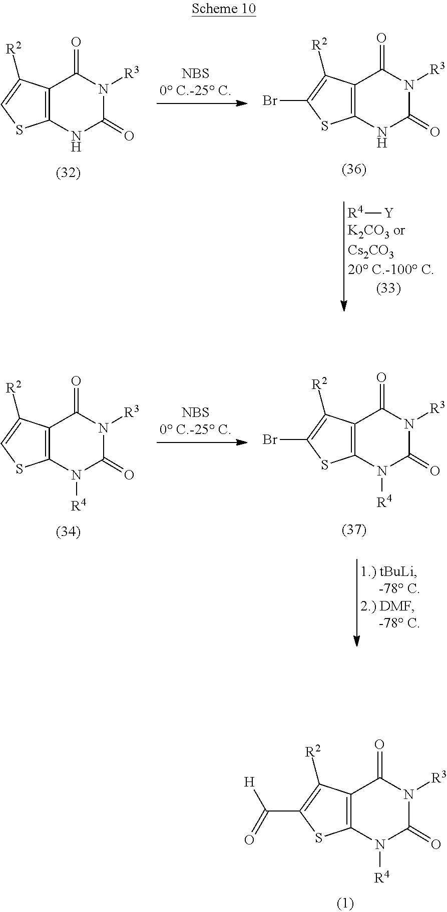

The thienouracils (32) or (34) are first converted here with a brominating agent to the brominated derivatives of the formula (36) or (37). By alkylation with a compound of the formula (33), the brominated thienouracils (36) can be converted to the derivatives of the formula (37). The synthesis sequence is completed by a halogen-metal exchange. Reaction of the metallated species thus generated in situ with a formamide gives the aldehydes of the formula (1). Examples of suitable brominating agents are N-bromosuccinimide (NBS) or elemental bromine; preference is given to NBS. The reaction is effected in an inert solvent, for example and with preference in dichloromethane or chloroform, within the temperature range between about 0° C. and room temperature. The alkylation of the compounds (36) to give the compounds (37) is effected under the same conditions as described above [see Scheme 9: conversion of (35) to (1) or of (32) to (34)]. The metallation of the 6-bromothienouracils (37) is preferably effected with tert-butyllithium in an ethereal solvent, such as preferably tetrahydrofuran, at a low temperature of about −78° C. At the same temperature, by addition of a formamide, preferably N,N-dimethylformamide (DMF), the aldehydes of the formula (1) are obtained.

Rather than the bromine derivatives, it is also possible to go via the corresponding chlorine or iodine derivatives in the above reaction sequence, these being obtainable from the compounds of the formula (32) or (34), for example through use of N-chlorosuccinimide (NCS), N-iodosuccinimide (NIS) or the elemental halogens (rather than NBS or bromine).

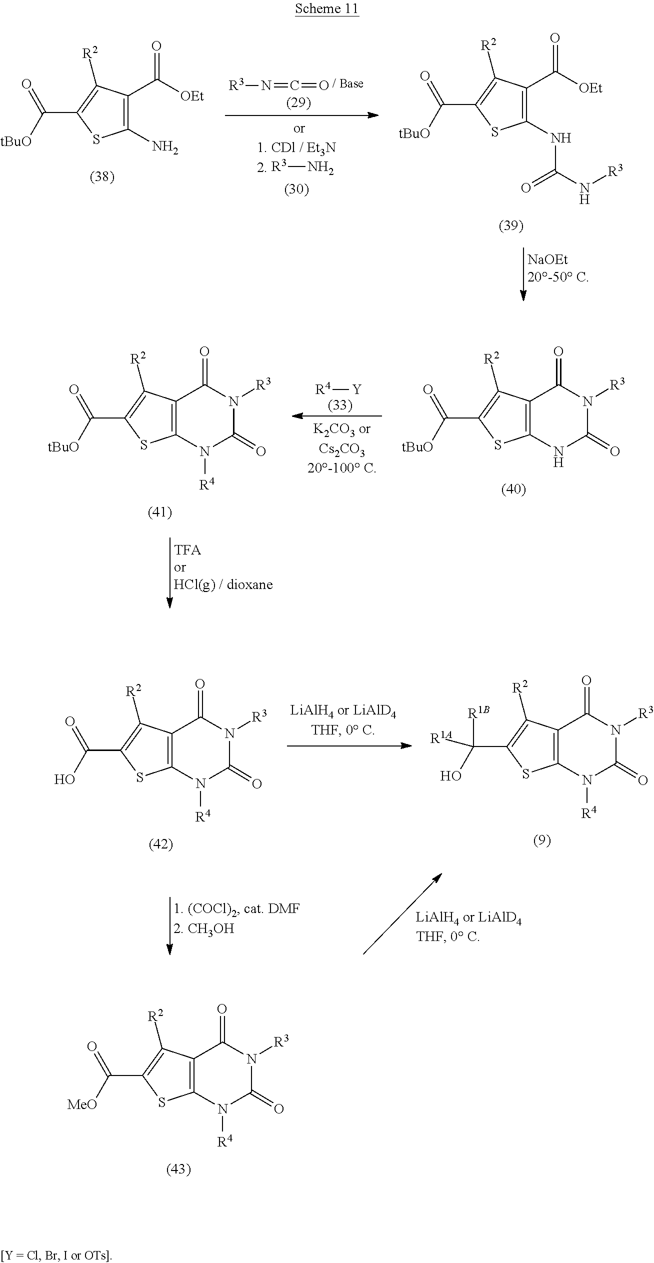

It is possible to obtain the thienouracil tert-butyl esters of the formula (41) from 5-aminothiophene-2,4-dicarboxylic esters of the formula (38) in an entirely analogous manner to the reactions described in Scheme 9 for the preparation of the intermediates (34). Subsequent treatment of the tert-butyl esters (41) at RT with either trifluoroacetic acid in dichloromethane or a solution of hydrogen chloride in 1,4-dioxane gives the carboxylic acids of the formula (42). These can either be converted to the alcohols of the formula (9) directly by reduction with lithium aluminium hydride [for R^{1A}═RIB=H] or lithium aluminium deuteride [for R^{1A}═RIB=D] at about 0° C. in an inert solvent, for example and with preference tetrahydrofuran (THF), or after prior conversion to the corresponding methyl esters of the formula (43). The latter can be obtained in a one-pot process by first converting the carboxylic acids of the formula (42) with oxalyl chloride in dichloromethane at RT in the presence of a catalytic amount of N,N-dimethylformamide (DMF) to the corresponding acid chlorides, which then give the methyl esters of the formula (43) by quenching with methanol.

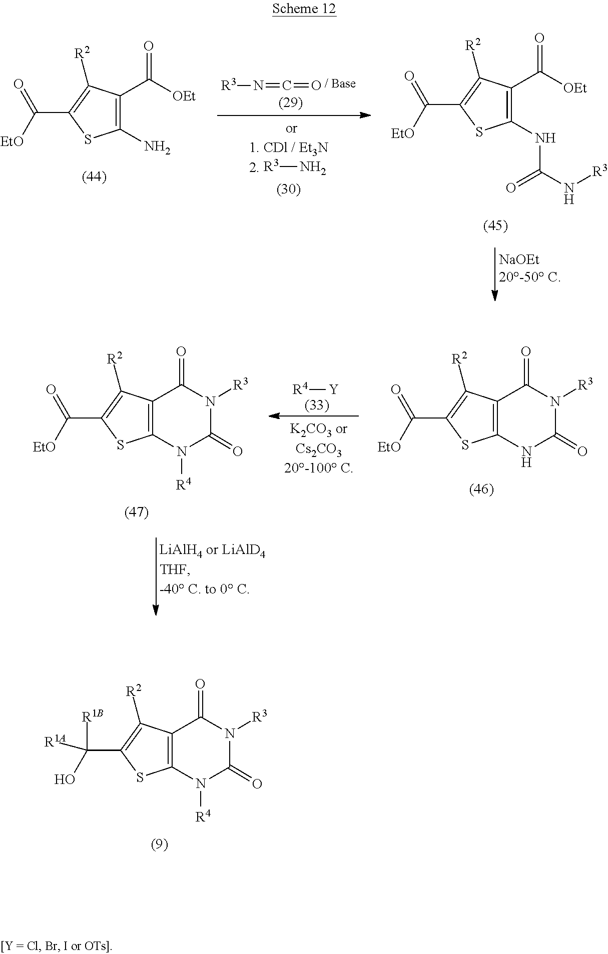

It is possible to obtain the thienouracil ethyl esters of the formula (47) from diethyl 5-aminothiophene-2,4-dicarboxylates of the formula (44), likewise in an entirely analogous manner to the reactions described in Scheme 9 for the preparation of the intermediates (34). The subsequent reduction with a complex metal hydride, for example and with preference lithium aluminium hydride [for R^{1A}═R^{1B}═H] or lithium aluminium deuteride [for R^{1A}═R^{1B}=D], then gives the alcohols of the formula (9) in a similar manner to that described above in Scheme 11. The reaction is effected typically in a temperature range between −40° C. and 0° C. in an inert solvent, for example and with preference tetrahydrofuran (THF).

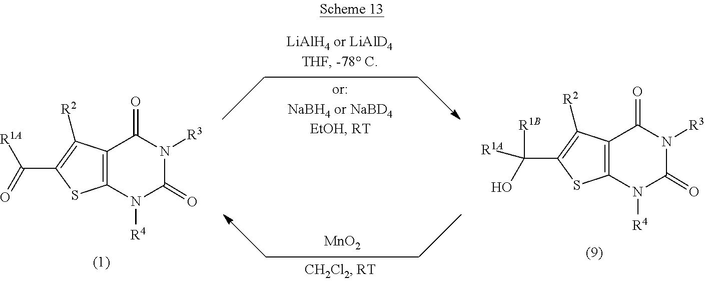

The aldehydes of the formula (1) and alcohols of the formula (9) obtained by one of the above-described processes can, if it seems desirable for synthesis purposes, be interconverted by several methods that are familiar to those skilled in the art. For example, the alcohols of the formula (9) can be oxidized with manganese dioxide in dichloromethane or with sulfur trioxide-pyridine complex in dimethyl sulfoxide (DMSO), in each case at RT, to the aldehydes of the formula (1). Conversely, the aldehydes of the formula (1) can be reduced with complex hydrides, for example lithium aluminium hydride, lithium aluminium deuteride, sodium borohydride or sodium borodeuteride, to the alcohols of the formula (9). The reduction with lithium aluminium hydride or lithium aluminium deuteride is preferably effected in tetrahydrofuran (THF) at −78° C., whereas the reduction with sodium borohydride or sodium borodeuteride can be effected, for example, in ethanol at RT. In this way, both the deuterated version of the aldehydes of the formula (1) [R^{1A}=D] and alcohols of the formula (9) in which one of the R^{1A} and R^{1B} radicals is hydrogen (¹H) and the other is deuterium (²H) are obtainable.

In addition, it is possible to obtain inventive compounds of the general formula (I) in which R^{1A} and/or R^{1B} is/are deuterium by proceeding from correspondingly deuterated aldehydes of the formula (1) [Schemes 1, 3, 4 and 5] or correspondingly deuterated alcohols of the formula (9) [Schemes 2, 3 and 7] or the deuterated intermediate (20) obtainable therefrom [Schemes 6 and 8], and using the corresponding deuterium variants of the complex metal hydrides specified therein (sodium borohydride, sodium triacetoxyborohydride or sodium cyanoborohydride), or using deuterium rather than hydrogen for the hydrogenation [Scheme 3].

Alkyl-substituted 2-aminothiophene-3-carboxylic esters such as the compounds of the formula (28) [Scheme 9, with R²=methyl or ethyl] and alkyl-substituted 5-aminothiophene-2,4-dicarboxylic esters such as the compounds of the formulae (38) and (44) [Schemes 11 and 12, with R²=methyl or ethyl] can be obtained by a known process via the 3-component reaction of acetone or 2-butanone or of an acetoacetic or 3-ketovaleric ester with an α-cyanoacetic ester and elemental sulfur [“Gewald reaction”; see, for example, B. P. McKibben et al., Tetrahedron Lett. 40, 5471-5474 (1999) and further literature cited therein].

The compounds of the formulae (2), (4), (5), (6), (7), (8), (10), (15), (16), (23), (25), (26), (29), (30) and (33) specified above are either commercially available or described as such in the literature, or they can be prepared from other commercially available compounds by literature methods familiar to those skilled in the art. Numerous detailed procedures and further literature references can also be found in the experimental section, in the section on the preparation of the starting compounds and intermediates.

The compounds of the invention have valuable pharmacological properties and can be used for prevention and treatment of diseases in humans and animals.

The compounds of the invention are potent and selective antagonists of the adenosine A2b receptor and are therefore suitable in particular for the treatment and/or prevention of disorders and pathological processes, especially those where the A2b receptor is involved in the course of an inflammatory event and/or tissue or vessel reconstruction.

In the context of the present invention, these include in particular disorders such as the group of the interstitial idiopathic pneumonias which includes idiopathic pulmonary fibrosis (IPF), acute interstitial pneumonia, non-specific interstitial pneumonias, lymphoid interstitial pneumonias, respiratory bronchiolitis with interstitial lung disease, cryptogenic organizing pneumonias, desquamative interstitial pneumonias and non-classifiable idiopathic interstitial pneumonias, furthermore granulomatous interstitial lung diseases, interstitial lung diseases of known aetiology and other interstitial lung diseases of unknown aetiology, pulmonary arterial hypertension (PAH) and other forms of pulmonary hypertension (PH), bronchiolitis obliterans syndrome (BOS), chronic-obstructive pulmonary disease (COPD), acute respiratory distress syndrome (ARDS), acute lung injury (ALI), alpha-1-antitrypsin deficiency (AATD), pulmonary emphysema (for example pulmonary emphysema induced by cigarette smoke), cystic fibrosis (CF), inflammatory and fibrotic disorders of the kidney, chronic intestinal inflammations (IBD, Crohn's disease, ulcerative colitis), peritonitis, peritoneal fibrosis, rheumatoid disorders, multiple sclerosis, inflammatory and fibrotic skin disorders, sickle cell anaemia and inflammatory and fibrotic eye disorders.

The compounds of the invention can additionally be used for treatment and/or prevention of asthmatic disorders of varying severity with intermittent or persistent characteristics (refractive asthma, bronchial asthma, allergic asthma, intrinsic asthma, extrinsic asthma, medicament- or dust-induced asthma), of various forms of bronchitis (chronic bronchitis, infectious bronchitis, eosinophilic bronchitis), of bronchiectasis, pneumonia, farmer's lung and related disorders, coughs and colds (chronic inflammatory cough, iatrogenic cough), inflammation of the nasal mucosa (including medicament-related rhinitis, vasomotoric rhinitis and seasonal allergic rhinitis, for example hay fever) and of polyps.

The compounds of the invention can additionally be used for treatment and/or prevention of cardiovascular disorders, for example high blood pressure (hypertension), heart failure, coronary heart disorders, stable and unstable angina pectoris, renal hypertension, peripheral and cardiovascular disorders, arrhythmias, rhythm disorders of the atria and ventricles, and conduction disorders, for example atrioventricular blocks of degrees I-III, supraventricular tachycardia, atrial fibrillation, atrial flutter, ventricular fibrillation, ventricular flutter, ventricular tachycardia, Torsade de pointes tachycardia, atrial and ventricular extrasystoles, AV-junctional extrasystoles, sick sinus syndrome, syncopes, AV nodal reentrant tachycardia, Wolff-Parkinson-White syndrome, acute coronary syndrome (ACS), autoimmune cardiac disorders (pericarditis, endocarditis, valvolitis, aortitis, cardiomyopathies), boxer cardiomyopathy, aneurysms, shock such as cardiogenic shock, septic shock and anaphylactic shock, and also for treatment and/or prevention of thromboembolic disorders and ischemias such as myocardial ischemia, myocardial infarction, stroke, cardiac hypertrophy, transient and ischemic attacks, preeclampsia, inflammatory cardiovascular disorders, spasms of the coronary arteries and peripheral arteries, edema formation such as, for example, pulmonary edema, cerebral edema, renal edema or edema caused by heart failure, peripheral circulatory disturbances, reperfusion damage, arterial and venous thromboses, microalbuminuria, myocardial insufficiency, endothelial dysfunction, micro- and macrovascular damage (vasculitis), and also to prevent restenoses, for example after thrombolysis therapies, percutaneous transluminal angioplasties (PTA), percutaneous transluminal coronary angioplasties (PTCA), heart transplants and bypass operations.

In the context of the present invention, the term “heart failure” encompasses both acute and chronic forms of heart failure, and also specific or related disease types thereof, such as acute decompensated heart failure, right heart failure, left heart failure, global failure, ischaemic cardiomyopathy, dilatative cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders and diastolic and systolic heart failure. The compounds of the invention are also suitable for treatment and/or prevention of renal disorders, in particular renal insufficiency and kidney failure. In the context of the present invention, the terms “renal insufficiency” and “kidney failure” encompass both acute and chronic manifestations thereof and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphatemia and/or need for dialysis. The present invention also encompasses the use of the compounds of the invention for treatment and/or prevention of sequelae of renal insufficiency, for example hypertension, pulmonary oedema, heart failure, uraemia, anaemia, electrolyte disturbances (for example hyperkalaemia, hyponatraemia) and disturbances in bone and carbohydrate metabolism.

In addition, the compounds of the invention are suitable for treatment and/or prevention of disorders of the urogenital system, for example benign prostate syndrome (BPS), benign prostate hyperplasia (BPH), benign prostate enlargement (BPE), bladder outlet obstruction (BOO), lower urinary tract syndromes (LUTS), neurogenic overactive bladder (OAB), incontinence, for example mixed urinary incontinence, urge urinary incontinence, stress urinary incontinence or overflow urinary incontinence (MUI, UUI, SUI, OUI), pelvic pain, and also erectile dysfunction and female sexual dysfunction.

In addition, the compounds according to the invention have antiinflammatory action and can therefore be used as antiinflammatory agents for the treatment and/or prevention of sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis, vulvovaginitis, rheumatoid disorders, inflammatory disorders of the central nervous system, multiple sclerosis, infammatory skin disorders and inflammatory eye disorders.

The compounds of the invention are also suitable for treatment and/or prevention of fibrotic disorders of the internal organs, for example the lung, the heart, the kidney, the bone marrow and in particular the liver, and also dermatological fibroses and fibrotic eye disorders. In the context of the present invention, the term “fibrotic disorders” includes in particular disorders such as hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, peritoneal fibrosis and similar fibrotic disorders, scleroderma, morphea, keloids, hypertrophic scarring, nevi, diabetic retinopathy, proliferative vitroretinopathy and disorders of the connective tissue (for example sarcoidosis). The compounds of the invention can likewise be used for promotion of wound healing, for controlling postoperative scarring, for example following glaucoma operations and cosmetically for ageing or keratinized skin.

The compounds of the invention can also be used for treatment and/or prevention of anemias such as hemolytic anemias, in particular hemoglobinopathies such as sickle cell anemia and thalassamias, megaloblastic anemias, iron deficiency anemias, anemias owing to acute blood loss, displacement anemias and aplastic anemias.

Moreover, the compounds according to the invention are suitable for the treatment of cancers such as, for example, skin cancer, brain tumours, head and neck tumours, oesophageal cancer, breast cancer, bone marrow tumours, leukaemias, liposarcomas, carcinomas of the gastrointestinal tract, of the liver, the pancreas, the lung, the kidney, the ureter, the prostate and the genital tract, bladder cancer and also of malignant tumours of the lymphoproliferative system, for example Hodgkin and Non-Hodgkin lymphoma.

In addition, the compounds of the invention can be used for treatment and/or prevention of arteriosclerosis, impaired lipid metabolism and dyslipidemias (hypolipoproteinemia, hypertriglyceridemias, hyperlipidemia, combined hyperlipidemias, hypercholesterolemia, abetalipoproteinemia, sitosterolemia), xanthomatosis, Tangier disease, adiposity, obesity, metabolic disorders (metabolic syndrome, hyperglycemia, insulin-dependent diabetes, non-insulin-dependent diabetes, gestation diabetes, hyperinsulinemia, insulin resistence, glucose intolerance and diabetic sequelae, such as retinopathy, nephropathy and neuropathy), of disorders of the gastrointestinal tract and the abdomen (glossitis, gingivitis, periodontitis, esophagitis, eosinophilic gastroenteritis, mastocytosis, Crohn's disease, colitis, proctitis, anus pruritis, diarrhea, celiac disease, hepatitis, hepatic fibrosis, cirrhosis of the liver, pancreatitis and cholecystitis), of disorders of the central nervous system and neurodegenerative disorders (stroke, Alzheimer's disease, Parkinson's disease, dementia, epilepsy, depressions, multiple sclerosis), immune disorders, thyroid disorders (hyperthyreosis), skin disorders (psoriasis, acne, eczema, neurodermitis, various forms of dermatitis, for example dermatitis abacribus, actinic dermatitis, allergic dermatitis, ammonia dermatitis, facticial dermatitis, autogenic dermatitis, atopic dermatitis, dermatitis calorica, dermatitis combustionis, dermatitis congelationis, dermatitis cosmetica, dermatitis escharotica, exfoliative dermatitis, dermatitis gangraenose, stasis dermatitis, dermatitis herpetiformis, lichenoid dermatitis, dermatitis linearis, dermatitis maligna, medicinal eruption dermatitis, dermatitis palmaris and plantaris, parasitic dermatitis, photoallergic contact dermatitis, phototoxic dermatitis, dermatitis pustularis, seborrhoeic dermatitis, sunburn, toxic dermatitis, Meleney's ulcer, dermatitis veneata, infectious dermatitis, pyrogenic dermatitis and perioral dermatitis, and also keratitis, bullosis, vasculitis, cellulitis, panniculitis, lupus erythematosus, erythema, lymphomas, skin cancer, Sweet syndrome, Weber-Christian syndrome, scar formation, wart formation, chilblains), of inflammatory eye diseases (saccoidosis, blepharitis, conjunctivitis, iritis, uveitis, chorioiditis, ophthalmitis), viral diseases (caused by influenza, adeno and corona viruses, for example HPV, HCMV, HIV, SARS), of disorders of the skeletal bone and the joints and also the skeletal muscle (various forms of arthritis, for example arthritis alcaptonurica, arthritis ankylosans, arthritis dysenterica, arthritis exsudativa, arthritis fungosa, arthritis gonorrhoica, arthritis mutilans, arthritis psoriatica, arthritis purulenta, arthritis rheumatica, arthritis serosa, arthritis syphilitica, arthritis tuberculosa, arthritis urica, arthritis villonodularis pigmentosa, atypical arthritis, hemophilic arthritis, juvenile chronic arthritis, rheumatoid arthritis and metastatic arthritis, and also Still syndrome, Felty syndrome, Sjörgen syndrome, Clutton syndrome, Poncet syndrome, Pott syndrome and Reiter syndrome, various forms of arthropathy, for example arthropathia deformans, arthropathia neuropathica, arthropathia ovaripriva, arthropathia psoriatica and arthropathia tabica, systemic scleroses, various forms of inflammatory myopathies, for example myopathie epidemica, myopathie fibrosa, myopathie myoglobinurica, myopathie ossificans, myopathie ossificans neurotica, myopathie ossificans progressiva multiplex, myopathie purulenta, myopathie rheumatica, myopathie trichinosa, myopathie tropica and myopathie typhosa, and also Giinther syndrome and Miinchmeyer syndrome), of inflammatory changes to the arteries (various forms of arteritis, for example endarteritis, mesarteritis, periarteritis, panarteritis, arteritis rheumatica, arteritis deformans, arteritis temporalis, arteritis cranialis, arteritis gigantocellularis and arteritis granulomatosa, and also Horton syndrome, Churg-Strauss syndrome and Takayasu arteritis), of Muckle-Well syndrome, of Kikuchi disease, of polychondritis, dermatosclerosis and also other disorders having an inflammatory or immunological component, for example cataract, cachexia, osteoporosis, gout, incontinence, lepra, Sezary syndrome and paraneoplastic syndrome, in the event of rejection reactions after organ transplants and for wound healing and angiogenesis particularly in the case of chronic wounds.

Because of their profile of properties, the compounds of the invention are particularly suitable for the treatment and/or prevention of interstitial lung diseases, especially idiopathic pulmonary fibrosis (IPF), and also of pulmonary hypertension (PH), bronchiolitis obliterans syndrome (BOS), chronic obstructive pulmonary disease (COPD), asthma, cystic fibrosis (CF), myocardial infarction, heart failure, haemoglobinopathies, here in particular sickle cell anaemia, and of cancers. The aforementioned well-characterized diseases in humans can also occur with comparable aetiology in other mammals and can likewise be treated therein with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention thus further provides for the use of the compounds of the invention for treatment and/or prevention of disorders, especially of the aforementioned disorders.

The present invention further provides for the use of the compounds of the invention for production of a medicament for treatment and/or prevention of disorders, especially of the aforementioned disorders.

The present invention further provides a medicament comprising at least one of the compounds of the invention for treatment and/or prevention of disorders, especially of the aforementioned disorders.

The present invention further provides for the use of the compounds of the invention in a method for treatment and/or prevention of disorders, especially of the aforementioned disorders.

The present invention further provides a process for treatment and/or prevention of disorders, especially of the aforementioned disorders, using an effective amount of at least one of the compounds of the invention.

The compounds of the invention can be used alone or, if required, in combination with one or more other pharmacologically active substances, provided that this combination does not lead to undesirable and unacceptable side effects. The present invention therefore further provides medicaments comprising at least one of the compounds of the invention and one or more further drugs, especially for treatment and/or prevention of the aforementioned disorders. Preferred examples of combination active ingredients suitable for this purpose include:
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat, nelociguat and vericiguat, and the compounds described in WO 00/06568, WO 00/06569, WO 02/42301, WO 03/095451, WO 2011/147809, WO 2012/004258, WO 2012/028647 and WO 2012/059549;
prostacyclin analogues and IP receptor agonists, by way of example and with preference iloprost, beraprost, treprostinil, epoprostenol or selexipag;
endothelin receptor antagonists, by way of example and with preference bosentan, darusentan, ambrisentan or sitaxsentan;
compounds which inhibit human neutrophile elastase (HNE), by way of example and with preference sivelestat or DX-890 (reltran);
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);
compounds which block the binding of serotonin to its receptors, by way of example and with preference antagonists of the 5-HT_{2B} receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, by way of example and with preference etomoxir, dichloroacetate, ranolazine or trimetazidine;
anti-obstructive agents as used, for example, for treatment of chronic obstructive pulmonary disease (COPD) or bronchial asthma, by way of example and with preference from the group of the inhalatively or systemically administered agonists of the beta-adrenergic receptor (beta-mimetics) and the inhalatively administered anti-muscarinergic substances;
antiinflammatory, immunomodulating, immunosuppressive and/or cytotoxic agents, by way of example and with preference from the group of the systemically or inhalatively administered corticosteroids and also acetylcysteine, montelukast, tipelukast, azathioprine, cyclophosphamide, hydroxycarbamide, azithromycin, IFN-γ, pirfenidone or etanercept;
antifibrotic agents, by way of example and with preference pirfenidone, lysophosphatidic acid receptor 1 (LPA-1) antagonists, sphingosine-1-phosphate receptor 3 (S1P3) antagonists, autotaxin inhibitors, FP receptor antagonists, lysyl oxidase (LOX) inhibitors, lysyl oxidase-like-2 inhibitors, vasoactive intestinal peptide (VIP), VIP analogues, αᵥβ₆-integrin antagonists, interferons, KCa3.1 blockers, CTGF inhibitors, IL-4 antagonists, IL-13 antagonists, TGF-β-antagonists, inhibitors of the WNT signalling pathway or CCR2 antagonists;
therapeutic antibodies and antibody-active ingredient conjugates, by way of example and with preference bevacizumab, cetuximab, trastuzumab, trastuzumab emtansin, brentuximab vedotin or anetumab ravtansin;
immunotherapeutic antibodies, by way of example and with preference ipilimumab, nivolumab, pembrolizumab (lambrolizumab), PF-06801591, pidilizumab, BMS-936559 (MDX-1105), atezolizumab, durvalumab, avelumab, MEDI-0680 or AMP-224;
antithrombotic agents, by way of example and with preference from the group of platelet aggregation inhibitors, the anticoagulants and the profibrinolytic substances;
hypotensive active ingredients, by way of example and with preference from the group of the calcium antagonists, angiotensin All antagonists, ACE inhibitors, vasopeptidase inhibitors, endothelin antagonists, renin inhibitors, alpha receptor blockers, beta receptor blockers, mineralocorticoid receptor antagonists and also the diuretics;
lipid metabolism modifiers, by way of example and with preference from the group of the thyroid receptor agonists, cholesterol synthesis inhibitors, by way of example and with preference HMG-CoA reductase or squalene synthesis inhibitors, of the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists; and/or
chemotherapeutics like those employed, for example, for the therapy of neoplasms in the lung or other organs.

In a preferred embodiment of the invention, the compounds of the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the compounds of the invention are administered in combination with an antimuscarinergic substance, by way of example and with preference ipratropium bromide, tiotropium bromide or oxitropium bromide.

In a preferred embodiment of the invention, the compounds of the invention are administered in combination with a corticosteroid, by way of example and with preference prednisone, prednisolone, methylprednisolone, triamcinolone, dexamethasone, beclomethasone, betamethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, melagatran, dabigatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin All antagonists, ACE inhibitors, endothelin antagonists, renin inhibitors, alpha receptor blockers, beta receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1 receptor blocker, by way of example and with preference prazosin.

In a preferred embodiment of the invention, the compounds of the invention are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin AII antagonist, by way of example and with preference losartan, candesartan, valsartan, telmisartan, irbesartan, olmesartan, eprosartan or azilsartan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone, eplerenone or finerenone.

In a preferred embodiment of the invention, the compounds of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment of the invention, the compounds of the invention are administered in combination with a CETP inhibitor, by way of example and with preference torcetrapib (CP-529 414), JJT-705 or CETP vaccine (Avant).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

Particular preference is given to combinations of the compounds of the invention with one or more further active ingredients selected from the group consisting of PDE 5 inhibitors, sGC activators, sGC stimulators, prostacyclin analogues, IP receptor agonists, endothelin antagonists, antifibrotic agents, antiinflammatory, immunomodulating, immunosuppressant and/or cytotoxic agents and/or compounds that inhibit the signal transduction cascade.

The present invention further provides medicaments which comprise at least one compound of the invention, typically together with one or more inert, non-toxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. take place intravenously, intraarterially, intracardially, intraspinally or intralumbally) or include an absorption (e.g. take place inhalatively, intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

Suitable for the other administration routes are, for example, pharmaceutical forms for inhalation (including powder inhalers, nebulizers, metered aerosols), nasal drops, solutions or sprays, throat sprays, tablets for lingual, sublingual or buccal administration, films/wafers or capsules, suppositories, eye drops, eye ointments or eyewashes, ocular inserts, ear drops, sprays, powders, washes or tampons, vaginal capsules, aqueous suspensions (lotions, shaking mixtures), lipophilic suspensions, emulsions, microemulsions, ointments, creams, transdermal therapeutic systems (e.g. patches), milk, pastes, foams, dusting powders, implants or stents.

Oral and parenteral administration are preferred, especially oral, intravenous and intrapulmonary (inhalative) administration.

The compounds of the invention can be converted to the administration forms mentioned. This can be done in a manner known per se, by mixing with inert, nontoxic, pharmaceutically suitable excipients. These excipients include
fillers and carriers (for example cellulose, microcrystalline cellulose, for example Avicel®, lactose, mannitol, starch, calcium phosphates, for example Di-Cafos®);
ointment bases (for example vaseline, paraffins, triglycerides, waxes, wool wax, wool wax alcohols, lanolin, hydrophilic ointment, polyethylene glycols);
suppository bases (for example polyethylene glycols, cocoa butter, hard fat);
solvents (e.g. water, ethanol, isopropanol, glycerol, propylene glycol, mid-chain triglycerides, fatty oils, liquid polyethylene glycols, paraffins);
surfactants, emulsifiers, dispersants or wetting agents (for example sodium dodecylsulfate, lecithin, phospholipids, fatty alcohols, for example Lanette®, sorbitan fatty acid esters, for example Span®, polyoxyethylene sorbitan fatty acid esters, for example Tween®, polyoxyethylene fatty acid glycerides, for example Cremophor®, polyoxyethylene fatty acid esters, polyoxyethylene fatty alcohol ethers, glycerol fatty acid esters, poloxamers, for example Pluronic®);
buffer substances, and also acids and bases (for example phosphates, carbonates, citric acid, acetic acid, hydrochloric acid, sodium hydroxide, ammonium carbonate, trometamol, triethanolamine);
isotonizing agents (for example glucose, sodium chloride);
adsorbents (for example finely divided silicas);
viscosity-increasing agents, gel formers, thickeners or binders (for example polyvinylpyrrolidone, methyl cellulose, hydroxypropyl cellulose, hydroxypropyl methylcellulose, carboxymethyl cellulose-sodium, starch, carbomers, polyacrylic acids, for example Carbopol®, alginates, gelatins);
disintegrants (for example modified starch, carboxymethyl cellulose-sodium, sodium starch glycolate, for example Explotab®, crosslinked polyvinylpyrrolidone, croscarmellose-sodium, for example AcDiSol®);
flow regulators, lubricants, glidants and mould release agents (for example magnesium stearate, stearic acid, talc, finely divided silicas, for example Aerosil®);
coating agents (for example sugar, shellac) and film formers for films or diffusion membranes with fast or modified dissolution (for example polyvinylpyrrolidones, for example Kollidon®, polyvinyl alcohol, ethyl cellulose, hydroxypropyl cellulose, hydroxypropyl methyl cellulose, hydroxypropyl methyl cellulose phthalate, cellulose acetate, cellulose acetate phthalate, polyacrylates, polymethacrylates, for example Eudragit®);
capsule materials (e.g. gelatins, hydroxypropyl methyl cellulose);
natural polymers (for example albumins);
synthetic polymers (for example polylactides, polyglycolides, polyacrylates, polymethacrylates, for example Eudragit®, polyvinylpyrrolidones, for example Kollidon®, polyvinyl alcohols, polyvinyl acetates, polyethylene oxides, polyethylene glycols and the copolymers and block copolymers thereof);
plasticizers (for example polyethylene glycols, propylene glycol, glycerol, triacetin, triacetyl citrate, dibutyl phthalate);
penetrants;
stabilizers (e.g. antioxidants, for example ascorbic acid, sodium ascorbate, ascorbyl palmitate, butylhydroxyanisole, butylhydroxytoluene, propyl gallate);
preservatives (for example parabens, sorbic acid, sodium benzoate, thiomersal, benzalkonium chloride, chlorhexidine acetate);
dyes (e.g. inorganic pigments, for example iron oxides, titanium dioxide);
aromas, sweeteners, flavour and/or odour correctors.

In general, it has been found to be advantageous in the case of parenteral administration to administer amounts of active compound of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight. In the case of intrapulmonary administration, the amount of active compound is generally about 0.1 to 50 mg per inhalation.

It may nevertheless be necessary in some cases to deviate from the stated amounts of active compounds, specifically as a function of body weight, route of administration, individual response to the active ingredient, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

### A. EXAMPLES

### Abbreviations and Acronyms

abs. absolute
Ac acetyl
aq. aqueous, aqueous solution
Boc tert-butoxycarbonyl
BPR backpressure regulator (in SFC-HPLC)
br broad (in NMR signal)
Ex. example
Bu butyl
c concentration
cat. catalytic
CDI N,N′-carbonyldiimidazole
CI chemical ionization (in MS)
conc. concentrated, concentrated solution
d doublet (in NMR)
d day(s)
TLC thin layer chromatography
DCI direct chemical ionization (in MS)
DCM dichloromethane
dd doublet of doublets (in NMR)
ddd doublet of doublet of doublets (NMR)
de diastereomeric excess
DIPEA N,N-diisopropylethylamine
dm doublet of multiplets (in NMR)
DME 1,2-dimethoxyethane
DMF N,N-dimethylformamide
DMSO dimethyl sulfoxide
dq doublet of quartets (in NMR)
dquin doublet of quintets (in NMR)
dt doublet of triplets (in NMR)
ΔT temperature increase, heating (of a reaction mixture)
dtd doublet of triplet of doublets (NMR)
ee enantiomeric excess
El electron impact ionization (in MS)
eq. equivalent(s)
ESI electrospray ionization (in MS)
Et ethyl
GC gas chromatography
GC/MS gas chromatography-coupled mass spectrometry
h hour(s)
HPLC high-pressure, high-performance liquid chromatography
iPr isopropyl
conc. concentrated (in the case of a solution)
LC liquid chromatography
LC/MS liquid chromatography-coupled mass spectrometry
lit. literature (reference)
m multiplet (in NMR)
M mol/l (concentration figure)
Me methyl
min minute(s)
MPLC medium-pressure liquid chromatography (on silica gel; also referred to as flash chromatography)
MS mass spectrometry
MWD multiwavelength detector
NBS N-bromosuccinimide
neg negative, negative ionization in MS
NMM N-methylmorpholine
NMO N-methylmorpholine N-oxide
NMP N-methyl-2-pyrrolidinone
NMR nuclear magnetic resonance spectrometry
Pd/C palladium on activated charcoal
PEG polyethylene glycol
Ph phenyl
pos positive, positive ionization in MS
Pr propyl
q quartet (in NMR)
quant. quantitative (in chemical yield)
quin quintet (in NMR)
R_{f} retention index (in TLC)
RP reverse phase (in HPLC)
RT room temperature
Rₜ retention time (in HPLC, LC/MS)
s singlet (in NMR)
sept septet (in NMR)
sext sextet (in NMR)
SFC supercritical liquid chromatography
t triplet (in NMR)
TBME tert-butyl methyl ether
tBu tert-butyl
td triplet of doublets (in NMR)
TFA trifluoroacetic acid
THF tetrahydrofuran
TMS tetramethylsilane
tq triplet of quartets (in NMR)
Ts para-toluenesulfonyl
tt triplet of triplets (in NMR)
UV ultraviolet spectrometry
Vol. volume
v/v volume to volume ratio (of a solution)

### HPLC, LC/MS and GC/MS Methods:

### Method 1 (LC/MS):

Instrument MS: Thermo Scientific FT-MS; UHPLC instrument: Thermo Scientific UltiMate 3000; column: Waters HSST3 C18 1.8 μm, 75 mm×2.1 mm; eluent A: 1 1 water+0.01% formic acid, eluent B: 1 1 acetonitrile+0.01% formic acid; gradient: 0.0 min 10% B→2.5 min 95% B→3.5 min 95% B; temperature: 50° C.; flow rate: 0.90 ml/min; UV detection: 210-300 nm.

### Method 2 (LC/MS):

Instrument: Waters Acquity SQD UPLC System; column: Waters Acquity UPLC HSS T3 1.8 μm, 50 mm×1 mm; eluent A: 1 1 water+0.25 ml 99% formic acid, eluent B: 1 1 acetonitrile+0.25 ml 99% formic acid; gradient: 0.0 min 90% A→1.2 min 5% A→2.0 min 5% A; temperature: 50° C.; flow rate: 0.40 ml/min; UV detection: 210-400 nm.

### Method 3 (LC/MS):

Instrument: Waters Acquity SQD UPLC System; column: Waters Acquity UPLC HSS T3 1.8 μm, 50 mm×1 mm; eluent A: 1 1 water+0.25 ml 99% formic acid, eluent B: 1 1 acetonitrile+0.25 ml 99% formic acid; gradient: 0.0 min 95% A→6.0 min 5% A→7.5 min 5% A; temperature: 50° C.; flow rate: 0.35 ml/min; UV detection: 210-400 nm.

### Method 4 (LC/MS):

Instrument: Waters Acquity UPLC-MS SingleQuad; column: Waters Acquity UPLC BEH C18 1.7 μm 50 mm×2.1 mm; eluent A: water+0.1% by vol. of formic acid (99%), eluent B: acetonitrile; gradient: 0.0-1.6 min 1-99% B, 1.6-2.0 min 99% B; flow rate: 0.8 ml/min; temperature: 60° C.; DAD scan: 210-400 nm.

### Method 5 (LC/MS):

Instrument: Micromass Quattro Premier with Waters UPLC Acquity; column: Thermo Hypersil GOLD 1.9 g 50×1 mm; eluent A: 1 1 water+0.5 ml 50% formic acid, eluent B: 1 1 acetonitrile+0.5 ml 50% formic acid; gradient: 0.0 min 97% A→0.5 min 97% A→3.2 min 5% A→4.0 min 5% A; temperature: 50° C.; flow rate: 0.30 ml/min; UV detection: 210 nm.

### Method 6 (LC/MS):

Instrument: Agilent MS Quad 6150 with HPLC Agilent 1290; column: Waters Acquity UPLC HSS T3 1.8 μm, 50 mm×2.1 mm; eluent A: 1 1 water+0.25 ml 99% formic acid, eluent B: 1 1 acetonitrile+0.25 ml 99% formic acid; gradient: 0.0 min 90% A→0.3 min 90% A→1.7 min 5% A→3.0 min 5% A; flow rate: 1.20 ml/min; temperature: 50° C.; UV detection: 205-305 nm.

### Method 7 (LC/MS):

Instrument: Waters Micromass Quattro Micro with HPLC Waters UPLC Acquity; column: Waters BEH C18 1.7 μm, 50 mm×2.1 mm; eluent A: 1 1 water+0.01 mol ammonium formate, eluent B: 1 1 acetonitrile; gradient: 0.0 min 95% A→0.1 min 95% A→2.0 min 15% A→2.5 min 15% A→2.51 min 10% A→3.0 min 10% A; flow rate: 0.5 ml/min; temperature: 40° C.; UV detection: 210 nm.

### Method 8 (LC/MS):

Instrument: Waters Single Quad MS System with Waters UPLC Acquity; column: Waters BEH C18 1.7 μm 50 mm×2.1 mm; eluent A: 1 1 water+1.0 ml aqueous ammonia (25%)/1, eluent B: 1 1 acetonitrile; gradient: 0.0 min 92% A→0.1 min 92% A→1.8 min 5% A→3.5 min 5% A; temperature: 50° C.; flow rate: 0.45 ml/min; UV detection: 210 nm (208-400 nm).

### Method 9 (GC-MS):

Instrument: Thermo DFS, Trace GC Ultra; column: Restek RTX-35, 15 m×200 μm×0.33 μm; constant flow rate of helium: 1.20 ml/min; oven: 60° C.; inlet: 220° C.; gradient: 60° C., 30° C./min→300° C. (hold for 3.33 min).

### Method 10 (Preparative HPLC):

Column: Chromatorex C18, 10 μm, 125 mm×30 mm; eluent: acetonitrile/water with 0.1% formic acid; gradient: 30:70→95:5 within 20 min.

### Method 11 (Preparative HPLC):

Column: Chromatorex C18, 10 μm, 125 mm×30 mm; eluent: acetonitrile/water with 0.1% formic acid; gradient: 20:80→95:5 within 20 min.

### Method 12 (Preparative HPLC):

Column: Chromatorex C18, 10 μm, 125 mm×30 mm; eluent: acetonitrile/water with 0.1% formic acid; gradient: 15:85→95:5 within 20 min.

### Method 13 (Preparative HPLC):

Column: Chromatorex C18, 10 μm, 250 mm×30 mm; eluent: acetonitrile/water with 0.1% trifluoroacetic acid; gradient: 10:90-95:5 within 30 min.

### Method 14 (Preparative HPLC):

Column: Chromatorex C18, 10 μm, 125 mm×30 mm; eluent: acetonitrile/water with 0.1% formic acid; gradient: 10:90→100:0 within 10 min.

Method 15 (preparative HPLC):

Column: Phenomenex Kinetex C18, 5 μm, 100 mm×30 mm; eluent A: water with 2% formic acid, eluent B: acetonitrile; gradient profile: 0 to 2 min 10% B, 2 to 2.2 min up to 30% B, 2.2 to 7 min up to 70% B, 7 to 7.5 min up to 92% B, 7.5 to 9 min 92% B; flow rate: 65 ml/min; room temperature; wavelength: 200-400 nm.

### Method 16 (Preparative HPLC):

Column: Reprosil-Pur C18, 10 μm, 250 mm×30 mm; eluent A: acetonitrile, eluent B: water with 0.05% trifluoroacetic acid; gradient: 0.0 min 10% A→4.25 min 10% A→4.5 min 40% A→11.5 min 60% A→12.0 min 100% A→14.5 min 100% A→14.75 min 20% A→18.0 min 20% A.

### Method 17 (Preparative HPLC):

Instrument: Waters Prep LC/MS-System; column: Phenomenex Kinetex C18, 5 μm, 100 mm×30 mm; eluent A: water with 2% formic acid, eluent B: acetonitrile; flow rate: 65 ml/min; gradient profile: 0 to 2 min 10% B, 2 to 2.2 min up to 20% B, 2.2 to 7 min up to 60% B, 7 to 7.5 min up to 92% B, 7.5 to 9 min 92% B; room temperature; wavelength: 200-400 nm.

### Method 18 (Preparative HPLC):

Instrument: Waters Prep LC/MS-System; column: XBridge C18, 5 μm, 100 mm×30 mm; eluent A: water with 2% formic acid, eluent B: acetonitrile; flow rate: 65 ml/min; gradient profile: 0 to 2 min 10% B, 2 to 2.2 min up to 20% B, 2.2 to 7 min up to 60% B, 7 to 7.5 min up to 92% B, 7.5 to 9 min 92% B; room temperature; wavelength: 200-400 nm.

### Further Details:

The descriptions of the coupling patterns of ¹H NMR signals which follow are guided by the visual appearance of the signals in question and do not necessarily correspond to a strict, physically correct interpretation. In general, the stated chemical shift refers to the centre of the signal in question; in the case of broad multiplets, an interval is generally given.

Melting points and melting point ranges, if stated, are uncorrected.

In cases where the reaction products were obtained by trituration, stirring or recrystallization, it was frequently possible to isolate further amounts of product from the respective mother liquor by chromatography. However, a description of this chromatography is dispensed with hereinbelow unless a large part of the total yield could only be isolated in this step.

All reactants or reagents whose preparation is not described explicitly hereinafter were purchased commercially from generally accessible sources. For all other reactants or reagents whose preparation is likewise not described hereinafter and which were not commercially obtainable or were obtained from sources which are not generally accessible, a reference is given to the published literature in which their preparation is described.

### Starting Compounds and Intermediates

### Example 1A

### Ethyl 2-[(cyclopropylcarbamoyl)amino]-4-methylthiophene-3-carboxylate

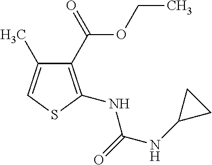 [SMILES: CCOC(=O)c1c(C)csc1NC(=O)NC1CC1]

### Process A:

To a solution of 3.0 g (15.7 mmol, 97% purity) of ethyl 2-amino-4-methylthiophene-3-carboxylate and 8.8 ml (62.8 mmol) of triethylamine in 80 ml of dichloromethane were added 5.09 g (31.4 mmol) of N,N′-carbonyldiimidazole (CDI), and the mixture was stirred at RT for 2 days. Then 2.2 ml (31.4 mmol) of cyclopropylamine were added. After a further 4 h at RT, the reaction mixture was transferred to a separating funnel and washed successively with about 50 ml each of water and saturated sodium chloride solution. After drying over anhydrous magnesium sulfate, the mixture was filtered and concentrated to dryness. The crude product that remained was purified by means of MPLC (Biotage Isolera One, SNAP KP-Sil cartridge, 100 g of silica gel, eluent: cyclohexane/ethyl acetate 1:1). After concentration of the product fractions and drying under high vacuum, 4.05 g (96% of theory) of the title compound were obtained.

¹H-NMR (400 MHz, DMSO-d₆, δ/ppm): 10.34 (broad, 1H), 7.90 (broad, 1H), 6.45 (s, 1H), 4.28 (q, 2H), 2.59-2.63 (m, 1H, partially concealed by DMSO signal), 2.27 (s, 3H), 1.31 (t, 3H), 0.69 (br. m, 2H), 0.46 (br. m, 2H).

LC/MS (Method 2, ESIpos): Rₜ=0.99 min, m/z=269 [M+H]⁺.

### Process B:

To a solution of 1.50 g (7.85 mmol, 97% purity) of ethyl 2-amino-4-methylthiophene-3-carboxylate in 7.5 ml of pyridine were added 1.31 g (15.7 mmol) of cyclopropyl isocyanate, and the mixture was stirred at 50° C. for 40 h. Subsequently, the mixture was concentrated to dryness. The remaining residue was taken up in a little dichloromethane and concentrated to dryness again. This procedure was repeated twice more. Final drying under high vacuum gave 2.25 g (100% of theory, 95% purity) of the title compound, which was used for subsequent reactions without further purification.

### Example 2A

### Ethyl 4-methyl-2-{[(1-methylcyclopropyl)carbamoyl]amino}thiophene-3-carboxylate

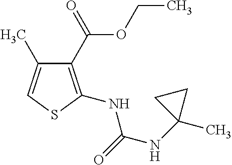

To a solution of 4.44 g (23.2 mmol) of ethyl 3-amino-4-methylthiophene-3-carboxylate in 133 ml of dichloromethane were added 7.54 g (46.5 mmol) of 1,1′-carbonyldiimidazole (CDI) and 13 ml (9.41 mmol) of triethylamine, and the mixture was stirred at RT for 2 days. Then 5.0 g (46.5 mmol) of 1-methylcyclopropanamine hydrochloride were added to the mixture, and the reaction mixture was stirred at RT for 4.5 h. The mixture was then washed successively with water and saturated sodium chloride solution. After drying over anhydrous magnesium sulfate, the mixture was filtered and concentrated. 6.87 g (98% of theory, 93% purity) of the title compound were obtained.

LC/MS (Method 2, ESIpos): Rₜ=1.04 min, m/z=283 [M+H]⁺.

### Example 3A

### Ethyl 4-methyl-2-({[1-(trifluoromethyl)cyclopropyl]carbamoyl}amino)thiophene-3-carboxylate

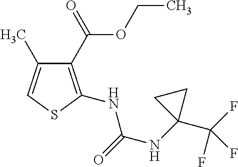 [SMILES: CCOC(=O)c1c(C)csc1NC(=O)NC1(C(F)(F)F)CC1]

Analogously to the process described in Ex. 1A, Process A, 2.96 g (16.0 mmol) of ethyl-2-amino-4-methylthiophene-3-dicarboxylate, 5.18 g (32.0 mmol) of CDI and 5.0 g (40.0 mmol) of 1-amino-1-(trifluoromethyl)cyclopropane were used to prepare 2.13 g (39% of theory) of the title compound.

¹H-NMR (600 MHz, DMSO-d₆, δ/ppm): 10.41 (br. s, 1H), 8.76 (br. s, 1H), 6.51 (s, 1H), 4.29 (q, 2H), 2.28 (d, 3H), 1.31 (t, 3H), 1.27 (br. s, 2H), 1.13 (br. s, 2H).

LC/MS (Method 1, ESIpos): Rₜ=2.05 min, m/z=337.08 [M+H]⁺.

### Example 4A

### Ethyl 4-methyl-2-{[(2-methylcyclopropyl)carbamoyl]amino}thiophene-3-carboxylate (trans racemate)

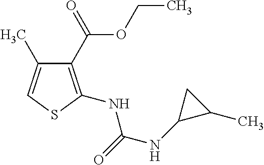

To a solution of 1.5 g (7.85 mmol) of ethyl 2-amino-4-methylthiophene-3-carboxylate in 7.5 ml of pyridine were added 1.61 g (15.7 mmol) of 1-isocyanato-2-methoxy-2-methylcyclopropane (trans racemate). The reaction mixture was stirred at 50° C. for 15 h. It was then concentrated to dryness on a rotary evaporator. The remaining residue was dissolved in dichloromethane and concentrated to dryness again. This material was then chromatographed using a silica gel cartridge (Biotage, 340 g of silica gel, eluent: hexane/ethyl acetate 95:5→60:40). 2.16 g (97% of theory) of the title compound were obtained.

¹H-NMR (400 MHz, DMSO-d₆, δ/ppm): 11.10-10.07 (m, 1H), 8.10-7.26 (m, 1H), 6.44 (s, 1H), 4.27 (q, 2H), 2.31-2.21 (m, 4H), 1.30 (t, 3H), 1.13-0.96 (m, 3H), 0.81 (br. s, 1H), 0.60 (br. s, 1H), 0.46 (br. s, 1H).

LC/MS (Method 4, ESIpos): Rₜ=1.29 min, m/z=282 [M+H]⁺.

### Example 5A

### Ethyl 2-{[(2,2-dimethylcyclopropyl)carbamoyl]amino}-4-methylthiophene-3-carboxylate (racemate)

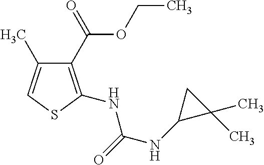

To a solution of 1.5 g (7.85 mmol) of ethyl 2-amino-4-methylthiophene-3-carboxylate in 7.5 ml of pyridine were added 1.84 g (15.71 mmol) of racemic 2-isocyanato-1,1-dimethylcyclopropane. The reaction mixture was stirred at 50° C. for 15 h. It was then concentrated to dryness on a rotary evaporator. The remaining residue was dissolved in dichloromethane and concentrated to dryness again. This material was then chromatographed using a silica gel cartridge (Biotage, 100 g of silica gel, eluent: hexane/ethyl acetate 95:5-60:40). 2.32 g (96% of theory) of the title compound were obtained.

¹H-NMR (400 MHz, DMSO-d₆, δ/ppm): 10.35 (br. s, 1H), 7.89 (br. s, 1H), 6.44 (br. s, 1H), 4.27 (q, 2H), 2.40-2.31 (m, 1H), 2.27 (s, 3H), 1.31 (t, 3H), 1.24-0.95 (m, 6H), 0.65 (br. s, 1H), 0.28 (br. s, 1H).

LC/MS (Method 4, ESIpos): Rₜ=1.36 min, m/z=297 [M+H]⁺.

### Example 6A

### Ethyl 2-{[(1-ethylcyclopropyl)carbamoyl]amino}-4-methylthiophene-3-carboxylate

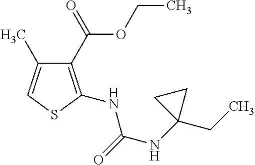 [SMILES: CCOC(=O)c1c(C)csc1NC(=O)NC1(CC)CC1]

Analogously to the process described in Ex. 1A, Process A, 1.09 g (5.87 mmol) of ethyl-2-amino-4-methylthiophene-3-dicarboxylate, 1.33 g (8.22 mmol) of CDI and 1.0 g (8.22 mmol) of 1-ethylcyclopropanamine were used to prepare 1.74 g (99% of theory) of the title compound. The reaction time after the addition of the amine in this case was 30 min. It was possible here to dispense with a chromatography purification; for purification, the crude product after the aqueous workup and the concentration was stirred with 20 ml of ethyl acetate at RT.

¹H-NMR (400 MHz, DMSO-d₆, δ/ppm): 10.21 (br. s, 1H), 8.10 (br. s, 1H), 6.43 (s, 1H), 4.28 (q, 2H), 2.27 (s, 3H), 1.52 (q, 2H), 1.31 (t, 3H), 0.89 (t, 3H), 0.64 (br. s, 4H).

LC/MS (Method 1, ESIpos): Rₜ=2.10 min, m/z=297.13 [M+H]⁺.

### Example 7A

### Ethyl 2-[(cyclobutylcarbamoyl)amino]-4-methylthiophene-3-carboxylate

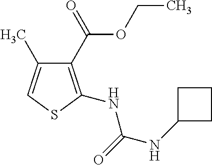 [SMILES: CCOC(=O)c1c(C)csc1NC(=O)NC1CCC1]

To a solution of 6.31 g (33.0 mmol, 97% purity) of ethyl 2-amino-4-methylthiophene-3-carboxylate and 18.4 ml (132 mmol) of triethylamine in 150 ml of dichloromethane were added 10.72 g (66.1 mmol) of CDI, and the mixture was stirred at RT for 2 days. Then 4.70 g (66.1 mmol) of cyclobutylamine were added. After a further 4 h at RT, the reaction mixture was transferred to a separating funnel and washed successively with about 100 ml each of water and saturated sodium chloride solution. After drying over anhydrous magnesium sulfate, the mixture was filtered and concentrated to dryness. The crude product that remained was purified by means of MPLC (Biotage Isolera One, SNAP KP-Sil cartridge, 340 g of silica gel, eluent: cyclohexane/ethyl acetate 5:1). After concentration of the product fractions and drying under high vacuum, 9.30 g (99% of theory) of the title compound were obtained.

¹H-NMR (400 MHz, DMSO-d₆, δ/ppm): 10.25 (s, 1H), 8.13 (br. d, 1H), 6.42 (s, 1H), 4.28 (q, 2H), 4.09 (sext, 1H), 2.26 (d, 3H), 2.24-2.15 (m, 2H), 1.94-1.79 (m, 2H), 1.72-1.54 (m, 2H), 1.31 (t, 3H).

LC/MS (Method 2, ESIpos): Rₜ=1.06 min, m/z=283 [M+H]⁺.

### Example 8A

### Ethyl 2-{[(3,3-difluorocyclobutyl)carbamoyl]amino}-4-methylthiophene-3-carboxylate

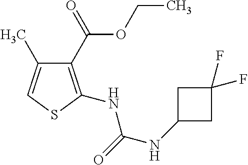 [SMILES: CCOC(=O)c1c(C)csc1NC(=O)NC1CC(F)(F)C1]

To a solution of 4.79 g (25.1 mmol, 97% purity) of ethyl 2-amino-4-methylthiophene-3-carboxylate in 144 ml of dichloromethane were added 8.13 g (50.2 mmol) of CDI and 14 ml (100 mmol) of triethylamine, and the mixture was stirred at RT for 3 days. Then 7.20 g (50.2 mmol) of 3,3-difluorocyclobutanamine hydrochloride were added, and stirring of the reaction mixture was continued at RT overnight. The mixture was then washed successively with about 200 ml each of water and saturated sodium chloride solution. After drying over anhydrous magnesium sulfate, the mixture was filtered and concentrated. The crude product was purified by means of MPLC (Biotage Isolera One, SNAP KP-Sil cartridge, silica gel, eluent: cyclohexane/ethyl acetate 2:1).

After combination of the product fractions, concentration and drying under high vacuum, 4.96 g (62% of theory) of the title compound were obtained.

¹H-NMR (400 MHz, DMSO-d₆, δ/ppm): 10.37 (s, 1H), 8.37 (d, 1H), 6.46 (s, 1H), 4.29 (q, 2H), 4.09-3.96 (m, 1H), 3.01-2.90 (m, 2H), 2.27 (s, 3H), 1.32 (t, 3H).

LC/MS (Method 1, ESIpos): Rₜ=1.99 min, m/z=319 [M+H]⁺.

### Example 9A

### Ethyl 4-methyl-2-[(oxetan-3-ylcarbamoyl)amino]thiophene-3-carboxylate

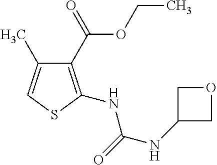 [SMILES: CCOC(=O)c1c(C)csc1NC(=O)NC1COC1]

Analogously to the process described in Ex. 7A, 3.0 g (16.2 mmol) of ethyl-2-amino-4-methylthiophene-3-dicarboxylate, 5.25 g (32.4 mmol) of CDI and 2.37 g (32.4 mmol) of oxetan-3-amine were used to prepare 4.39 g (94% of theory) of the title compound.

¹H-NMR (400 MHz, DMSO-d₆, δ/ppm): 10.39 (s, 1H), 8.62 (br. d, 1H), 6.45 (s, 1H), 4.83-4.69 (m, 3H), 4.46-4.37 (m, 2H), 4.29 (q, 2H), 2.27 (s, 3H), 1.32 (t, 3H).

LC/MS (Method 1, ESIpos): Rₜ=1.61 min, m/z=285.09 [M+H]⁺.

### Example 10A

### Ethyl 4-methyl-2-{[(1-methylcyclobutyl)carbamoyl]amino}thiophene-3-carboxylate

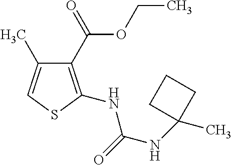

Analogously to the process described in Ex. 7A, 4.0 g (21.6 mmol) of ethyl-2-amino-4-methylthiophene-3-dicarboxylate, 5.25 g (32.4 mmol) of CDI and 3.68 g (43.2 mmol) of 1-methylcyclobutanamine were used to prepare 6.23 g (97% of theory) of the title compound. The reaction time after addition of the amine here was 1 h.

¹H-NMR (400 MHz, DMSO-d₆, δ/ppm): 10.23 (s, 1H), 7.99 (s, 1H), 6.41 (s, 1H), 4.28 (q, 2H), 2.32-2.20 (m, 2H), 2.26 (s, 3H), 1.95-1.84 (m, 2H), 1.84-1.70 (m, 2H), 1.39 (s, 3H), 1.31 (t, 3H).

LC/MS (Method 1, ESIpos): Rₜ=2.16 min, m/z=297.13 [M+H]⁺.

### Example 11A

### Ethyl 2-{[(trans-3-methoxycyclobutyl)carbamoyl]amino}-4-methylthiophene-3-carboxylate

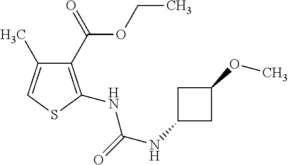

To a solution of 3.36 g (18.2 mmol) of ethyl 2-amino-4-methylthiophene-3-carboxylate in 105 ml of dichloromethane were added 4.42 g (27.3 mmol) of CDI and 10 ml (72.7 mmol) of triethylamine, and the mixture was stirred at RT for 4 days. Then 5.0 g (36.34 mmol) of trans-3-methoxycyclobutanamine hydrochloride were added to the mixture, and the reaction mixture was stirred at RT for a further 2 h. The mixture was then washed successively with water and saturated sodium chloride solution. After drying over anhydrous magnesium sulfate, the mixture was filtered and concentrated. 5.61 g (99% of theory) of the title compound were obtained.

¹H-NMR (400 MHz, DMSO-d₆, δ/ppm): 10.30 (s, 1H), 8.18 (br. s, 1H), 6.42 (s, 1H), 4.28 (q, 2H), 4.18-4.07 (m, 1H), 3.98-3.93 (m, 1H), 3.14 (s, 3H), 2.54 (s, 3H), 2.28-2.19 (m, 2H), 2.13-2.05 (m, 2H), 1.31 (t, 3H).

LC/MS (Method 1, ESIpos): Rₜ=1.84 min, m/z=313 [M+H]⁺.

### Example 12A

### Ethyl 2-{[(cis-3-methoxycyclobutyl)carbamoyl]amino}-4-methylthiophene-3-carboxylate

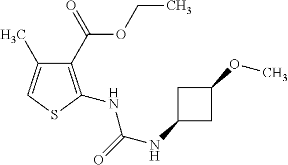

To a solution of 3.37 g (18.2 mmol) of ethyl 2-amino-4-methylthiophene-3-carboxylate in 105 ml of dichloromethane were added 4.42 g (27.3 mmol) of CDI and 10 ml (73.0 mmol) of triethylamine, and the mixture was stirred at RT for 5 days. Then 5.0 g (36.3 mmol) of cis-3-methoxycyclobutanamine hydrochloride were added to the mixture, and the reaction mixture was stirred at RT for a further 2 h. The mixture was then washed successively with water and saturated sodium chloride solution. After drying over anhydrous magnesium sulfate, the mixture was filtered and concentrated. 5.89 g (100% of theory) of the title compound were obtained.

¹H-NMR (400 MHz, DMSO-d₆, δ/ppm): 10.27 (s, 1H), 8.10 (br. s, 1H), 6.42 (s, 1H), 4.28 (q, 2H), 3.76-3.63 (m, 1H), 3.59-3.52 (m, 1H), 3.13 (s, 3H), 2.61-2.57 (m, 2H), 2.26 (d, 3H), 1.74-1.67 (m, 2H), 1.31 (t, 3H).

LC/MS (Method 2, ESIpos): Rₜ=0.97 min, m/z=313 [M+H]⁺.

### Example 13A

### Ethyl 2-{[(3,3-dimethylcyclobutyl)carbamoyl]amino}-4-methylthiophene-3-carboxylate

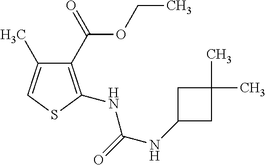

To a solution of 3.41 g (18.4 mmol) of ethyl 2-amino-4-methylthiophene-3-carboxylate in 105 ml of dichloromethane were added 4.48 g (27.7 mmol) of CDI and 10 ml (74.0 mmol) of triethylamine, and the mixture was stirred at RT for 3 days. Then 5 g (36.9 mmol) of 3,3-dimethylcyclobutanamine hydrochloride were added to the mixture, and the reaction mixture was stirred at RT for a further 2 h. The mixture was then washed successively with water and saturated sodium chloride solution. After drying over anhydrous magnesium sulfate, the mixture was filtered and concentrated. 6.93 g (100% of theory, 86% purity) of the title compound were obtained.

¹H-NMR (400 MHz, DMSO-d₆, δ/ppm): 10.26 (s, 1H), 8.08 (br. s, 1H), 6.41 (s, 1H), 4.28 (q, 2H), 4.14-4.04 (m, 1H), 2.26 (s, 3H), 2.11-2.06 (m, 2H), 1.69-1.64 (m, 2H), 1.31 (t, 3H), 1.12 (s, 3H), 1.10 (s, 3H).

LC/MS (Method 1, ESIpos): Rₜ=2.29 min, m/z=311 [M+H]⁺.

### Example 14A

### Ethyl 4-methyl-2-[(spiro[3.3]hept-2-ylcarbamoyl)amino]thiophene-3-carboxylate

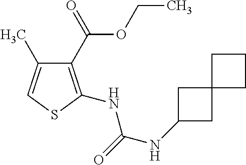 [SMILES: CCOC(=O)c1c(C)csc1NC(=O)NC1CC2(CCC2)C1]

Analogously to the process described in Ex. 7A, 3.14 g (16.9 mmol) of ethyl-2-amino-4-methylthiophene-3-carboxylate, 4.12 g (25.4 mmol) of CDI and 5.0 g (33.9 mmol) of spiro[3.3]heptan-2-amine hydrochloride were used to prepare 5.35 g (98% of theory) of the title compound. The reaction time after addition of the amine hydrochloride here was 1 h.

¹H-NMR (400 MHz, DMSO-d₆, δ/ppm): 10.24 (s, 1H), 8.06 (br. d, 1H), 6.41 (s, 1H), 4.28 (q, 2H), 3.94 (sext, 1H), 2.36-2.28 (m, 2H), 2.26 (d, 3H), 2.04-1.96 (m, 2H), 1.94-1.87 (m, 2H), 1.86-1.72 (m, 4H), 1.31 (t, 3H).

LC/MS (Method 2, ESIpos): Rₜ=1.24 min, m/z=323 [M+H]⁺.

### Example 15A

### Ethyl 2-[(cyclopentylcarbamoyl)amino]-4-methylthiophene-3-carboxylate

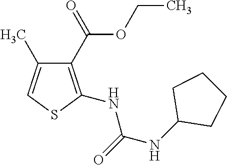 [SMILES: CCOC(=O)c1c(C)csc1NC(=O)NC1CCCC1]

To a solution of 4.0 g (21.6 mmol) of ethyl 2-amino-4-methylthiophene-3-carboxylate and 12 ml (86.4 mmol) of triethylamine in 120 ml of dichloromethane were added 5.25 g (32.4 mmol) of CDI, and the mixture was stirred at RT for 2 days. Then 4.3 ml (43.2 mmol) of cyclopentylamine were added. After stirring at RT for a further hour, the reaction mixture was transferred to a separating funnel and washed successively with about 100 ml each of water and saturated sodium chloride solution. After drying over anhydrous magnesium sulfate, the mixture was filtered and concentrated to dryness. The crude product that remained was purified by means of MPLC (Biotage Isolera One, SNAP KP-Sil cartridge, 340 g of silica gel, eluent: cyclohexane/ethyl acetate 2:1). After concentration of the product fractions and drying under high vacuum, 5.96 g (93% of theory) of the title compound were obtained.

¹H-NMR (400 MHz, DMSO-d₆, δ/ppm): 10.26 (s, 1H), 7.86 (br. d, 1H), 6.40 (s, 1H), 4.27 (q, 2H), 3.93 (sext, 1H), 2.26 (s, 3H), 1.82 (dq, 2H), 1.71-1.59 (m, 2H), 1.58-1.47 (m, 2H), 1.45-1.35 (m, 2H), 1.31 (t, 3H).

LC/MS (Method 2, ESIpos): Rₜ=1.13 min, m/z=297 [M+H]⁺.

### Example 16A

### 3-Cyclopropyl-5-methylthieno[2,3-d]pyrimidine-2,4(1H,3H)-dione

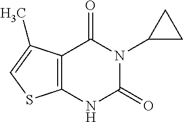

13.51 g (50.3 mmol) of the compound from Ex. 1A were dissolved in 160 ml of ethanol, and 37.6 ml (101 mmol) of a 21% solution of sodium ethoxide in ethanol were added. The reaction mixture was stirred first at RT for about 15 h and then at 50° C. for 2 h. Thereafter, the reaction mixture was acidified by adding 1 M hydrochloric acid. In the course of this, the product precipitated out. The product was filtered off with suction, washed to neutrality with water and dried under high vacuum. In this way, 10.95 g (97% of theory) of the title compound were obtained.

¹H-NMR (400 MHz, DMSO-d₆, δ/ppm): 11.92 (s, 1H), 6.64 (d, 1H), 2.56-2.50 (m, 1H, partially concealed by DMSO signal), 2.33 (d, 3H), 1.09-0.90 (m, 2H), 0.76-0.60 (m, 2H).

LC/MS (Method 2, ESIpos): Rₜ=0.60 min, m/z=223 [M+H]⁺.

### Example 17A

### 5-Methyl-3-(1-methylcyclopropyl)thieno[2,3-d]pyrimidine-2,4(1H,3H)-dione

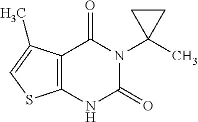 [SMILES: Cc1csc2[nH]c(=O)n(C3(C)CC3)c(=O)c12]

6.87 g (22.7 mmol, 93% purity) of the compound from Ex. 2A were dissolved in 214 ml of ethanol, and 12.7 ml (34.0 mmol) of sodium ethoxide solution (21% by weight in ethanol) were added. The reaction mixture was stirred first at RT for 16 h and then at 50° C. for 16 h. The mixture was then poured onto ice-water and adjusted to pH 5 with acetic acid. The precipitated solids were filtered off, washed to neutrality with water and suction-dried (=1st fraction of the title compound). The mother liquor was extracted with ethyl acetate, and the organic phase was washed with saturated sodium chloride solution. After drying over anhydrous magnesium sulfate, the mixture was filtered and concentrated (=2nd fraction of the title compound). In this way, a total of 4.30 g (77% of theory, 95% purity) of the title compound were obtained.

¹H-NMR (400 MHz, DMSO-d₆, δ/ppm): 11.90 (br. s, 1H), 6.64 (d, 1H), 2.34 (d, 3H), 1.32 (s, 3H), 0.90-0.78 (m, 4H).

LC/MS (Method 2, ESIpos): Rₜ=0.67 min, m/z=237 [M+H]⁺.

### Example 18A

### 5-Methyl-3-[1-(trifluoromethyl)cyclopropyl]thieno[2,3-d]pyrimidine-2,4(1H,3H)-dione

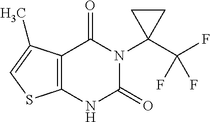 [SMILES: Cc1csc2[nH]c(=O)n(C3(C(F)(F)F)CC3)c(=O)c12]

2.12 g (6.30 mmol) of the compound from Ex. 3A were dissolved in 30 ml of ethanol, and 4.7 ml (12.6 mmol) of a 21% solution of sodium ethoxide in ethanol were added. The reaction mixture was stirred at RT for about 16 h. This was followed by acidification by adding 1 M hydrochloric acid. In the course of this, the product precipitated out. The product was filtered off with suction, washed to neutrality with water and dried under high vacuum. 1.67 g (91% of theory) of the title compound were obtained.

¹H-NMR (400 MHz, DMSO-d₆, δ/ppm): 12.16 (s, 1H), 6.70 (d, 1H), 2.34 (d, 3H), 1.65-1.46 (m, 2H), 1.41-1.26 (m, 2H).

LC/MS (Method 2, ESIpos): Rₜ=1.43 min, m/z=291 [M+H]⁺.

### Example 19A

### 5-Methyl-3-(2-methylcyclopropyl)thieno[2,3-d]pyrimidine-2,4(1H,3H)-dione (trans racemate)

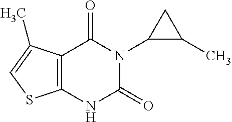 [SMILES: Cc1csc2[nH]c(=O)n(C3CC3C)c(=O)c12]

2.15 g (7.63 mmol) of the compound from Ex. 4A were dissolved in 25 ml of ethanol, and 5.7 ml (15.3 mmol) of a 21% solution of sodium ethoxide in ethanol were added. After the mixture had been stirred at RT for about 20 h, 17.5 ml of 1 M hydrochloric acid were added at RT. The resulting precipitate was filtered off with suction, washed with water until neutral and dried. 1.72 g (95% of theory) of the title compound were obtained.

¹H-NMR (400 MHz, DMSO-d₆, δ/ppm): 11.93 (br. s, 1H), 6.68-6.59 (m, 1H), 2.33 (d, 3H), 2.17 (dt, 1H), 1.14 (d, 3H), 1.05-0.92 (m, 1H), 0.89-0.74 (m, 2H).

LC/MS (Method 4, ESIpos): Rₜ=0.89 min, m/z=237 [M+H]⁺.

### Example 20A

### 3-(2,2-Dimethylcyclopropyl)-5-methylthieno[2,3-d]pyrimidine-2,4(1H,3H)-dione (racemate)

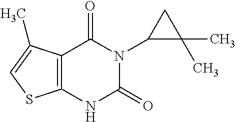 [SMILES: Cc1csc2[nH]c(=O)n(C3CC3(C)C)c(=O)c12]

2.31 g (7.81 mmol) of the compound from Ex. 5A were dissolved in 21.3 ml of ethanol, and 5.8 ml (15.6 mmol) of a 21% solution of sodium ethoxide in ethanol were added. After the mixture had been stirred at RT for 62 h, 18 ml of 1 M hydrochloric acid were added at RT. The resulting precipitate was filtered off with suction, washed with water until neutral and dried. 1.69 g (84% of theory) of the title compound were obtained.

¹H-NMR (400 MHz, DMSO-d₆, δ/ppm): 11.95 (br. s, 1H), 6.64 (d, 1H), 2.36-2.29 (m, 4H), 1.16 (s, 3H), 1.01 (dd, 1H), 0.86 (s, 3H), 0.78-0.72 (m, 1H).

LC/MS (Method 4, ESIpos): Rₜ=0.99 min, m/z=251 [M+H]⁺.

### Example 21A

### 3-(1-Ethylcyclopropyl)-5-methylthieno[2,3-d]pyrimidine-2,4(1H,3H)-dione

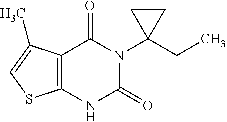 [SMILES: CCC1(n2c(=O)[nH]c3scc(C)c3c2=O)CC1]

1.73 g (5.84 mmol) of the compound from Ex. 6A were dissolved in 20 ml of ethanol, and 3.9 ml (10.5 mmol) of a 21% solution of sodium ethoxide in ethanol were added. The reaction mixture was stirred at 50° C. for about 16 h. Thereafter, the mixture was concentrated to about half of its original volume and then acidified by adding 1 M hydrochloric acid. In the course of this, the product precipitated out. The product was filtered off with suction, washed to neutrality with water and dried under high vacuum. 1.27 g (86% of theory) of the title compound were obtained.

¹H-NMR (400 MHz, DMSO-d₆, δ/ppm): 11.88 (s, 1H), 6.63 (d, 1H), 2.33 (d, 3H), 1.68 (qd, 2H), 0.98-0.91 (m, 1H), 0.91-0.86 (m, 1H), 0.85-0.77 (m, 2H), 0.81 (t, 3H).

LC/MS (Method 1, ESIpos): Rₜ=1.43 min, m/z=251.08 [M+H]⁺.

### Example 22A

### 3-Cyclobutyl-5-methylthieno[2,3-d]pyrimidine-2,4(1H,3H)-dione

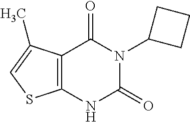 [SMILES: Cc1csc2[nH]c(=O)n(C3CCC3)c(=O)c12]

9.20 g (32.6 mmol) of the compound from Ex. 7A were dissolved in 90 ml of ethanol, and 24.3 ml (65.2 mmol) of a 21% solution of sodium ethoxide in ethanol were added. The reaction mixture was stirred first at RT for about 15 h and then at 50° C. for 1 h. Thereafter, the reaction mixture was acidified by adding 1 M hydrochloric acid. In the course of this, the product precipitated out. The product was filtered off with suction, washed to neutrality with water and dried under high vacuum. 6.48 g (84% of theory) of the title compound were obtained.

¹H-NMR (400 MHz, DMSO-d₆, δ/ppm): 12.00 (s, 1H), 6.65 (d, 1H), 5.23 (quin, 1H), 2.98-2.80 (m, 2H), 2.34 (d, 3H), 2.12 (qt, 2H), 1.88-1.61 (m, 2H).

LC/MS (Method 2, ESIpos): Rₜ=0.80 min, m/z=237 [M+H]⁺.

### Example 23A

### 3-(3,3-Difluorocyclobutyl)-5-methylthieno[2,3-d]pyrimidine-2,4(1H,3H)-dione

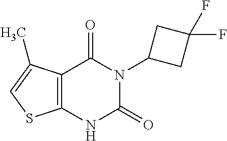 [SMILES: Cc1csc2[nH]c(=O)n(C3CC(F)(F)C3)c(=O)c12]

4.96 g (14.5 mmol) of the compound from Ex. 8A were dissolved in 137 ml of ethanol, and 7 ml of sodium ethoxide solution (21% by weight in ethanol) were added. The reaction mixture was stirred at RT for 3 h and then added to ice-water and adjusted to pH 5 with acetic acid. The precipitated solids were filtered off, washed to neutrality with water and suction-dried. 3.92 g (99% of theory) of the title compound were obtained.

¹H-NMR (400 MHz, DMSO-d₆, δ/ppm): 12.15 (s, 1H), 6.69 (d, 1H), 5.14 (q, 1H), 3.58-3.46 (m, 2H), 2.86-2.76 (m, 2H), 2.34 (d, 3H).

LC/MS (Method 1, ESIpos): Rₜ=1.56 min, m/z=273 [M+H]⁺.

### Example 24A

### 5-Methyl-3-(oxetan-3-yl)thieno[2,3-d]pyrimidine-2,4(1H,3H)-dione

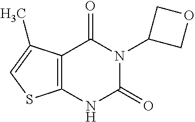 [SMILES: Cc1csc2[nH]c(=O)n(C3COC3)c(=O)c12]

Analogously to the process described in Ex. 18A, 4.30 g (15.1 mmol) of the compound from Ex. 9A were used to prepare 3.04 g (84% of theory) of the title compound. The reaction time here was 1 h.

¹H-NMR (400 MHz, DMSO-d₆, δ/ppm): 12.14 (s, 1H), 6.69 (s, 1H), 5.07-4.94 (m, 1H), 4.78-4.71 (m, 2H), 4.70-4.64 (m, 2H), 2.32 (s, 3H).

LC/MS (Method 1, ESIpos): Rₜ=0.96 min, m/z=239.05 [M+H]⁺.

### Example 25A

### 5-Methyl-3-(1-methylcyclobutyl)thieno[2,3-d]pyrimidine-2,4(1H,3H)-dione

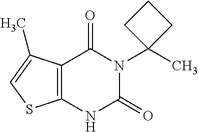 [SMILES: Cc1csc2[nH]c(=O)n(C3(C)CCC3)c(=O)c12]

Analogously to the process described in Ex. 16A, 6.23 g (21.0 mmol) of the compound from Ex. 10A were used to prepare 4.51 g (86% of theory) of the title compound.

¹H-NMR (400 MHz, DMSO-d₆, δ/ppm): 11.83 (s, 1H), 6.63 (d, 1H), 2.39-2.20 (m, 4H), 2.31 (d, 3H), 1.80-1.57 (m, 2H), 1.51 (s, 3H).

LC/MS (Method 1, ESIpos): Rₜ=1.54 min, m/z=251.08 [M+H]⁺.

### Example 26A

### 3-(trans-3-Methoxycyclobutyl)-5-methylthieno[2,3-d]pyrimidine-2,4(1H,3H)-dione

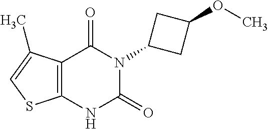 [SMILES: CO[C@H]1C[C@H](n2c(=O)[nH]c3scc(C)c3c2=O)C1]

5.61 g (18.0 mmol) of the compound from Ex. 11A were dissolved in 170 ml of ethanol, and 10 ml of sodium ethoxide solution (21% by weight in ethanol) were added. The reaction mixture was stirred at RT overnight, then added to ice-water, adjusted to pH 5 with acetic acid and extracted with dichloromethane. The organic phase was washed with water and saturated sodium chloride solution. After drying over anhydrous magnesium sulfate, the mixture was filtered and concentrated. 4.48 g (92% of theory) of the title compound were obtained.

¹H-NMR (400 MHz, DMSO-d₆, δ/ppm): 12.06 (s, 1H), 6.67 (d, 1H), 5.51-5.43 (m, 1H), 4.15-4.08 (m, 1H), 3.16 (s, 3H), 2.98-2.91 (m, 2H), 2.34 (d, 3H), 2.23-2.17 (m, 2H).

LC/MS (Method 2, ESIpos): Rₜ=0.71 min, m/z=267 [M+H]⁺.

### Example 27A

### 3-(cis-3-Methoxycyclobutyl)-5-methylthieno[2,3-d]pyrimidine-2,4(1H,3H)-dione

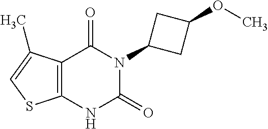 [SMILES: CO[C@H]1C[C@@H](n2c(=O)[nH]c3scc(C)c3c2=O)C1]

5.89 g (18.9 mmol) of the compound from Ex. 12A were dissolved in 180 ml of ethanol, and 11 ml of sodium ethoxide solution (21% by weight in ethanol) were added. The reaction mixture was stirred at RT overnight, then added to ice-water, adjusted to pH 5 with acetic acid and extracted with dichloromethane. The organic phase was washed with water and saturated sodium chloride solution. After drying over anhydrous magnesium sulfate, the mixture was filtered and concentrated. 4.65 g (92% of theory) of the title compound were obtained.

¹H-NMR (400 MHz, DMSO-d₆, δ/ppm): 12.01 (s, 1H), 6.67 (d, 1H), 4.75-4.66 (m, 1H), 3.67-3.60 (m, 1H), 3.16 (s, 3H), 2.83-2.76 (m, 2H), 2.54 (s, 3H), 2.52-2.44 (m, 2H).

LC/MS (Method 2, ESIpos): Rₜ=1.27 min, m/z=267 [M+H]⁺.

### Example 28A

### 3-(3,3-Dimethylcyclobutyl)-5-methylthieno[2,3-d]pyrimidine-2,4(1H,3H)-dione

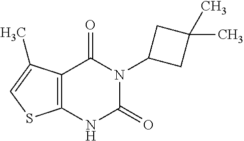 [SMILES: Cc1csc2[nH]c(=O)n(C3CC(C)(C)C3)c(=O)c12]

6.92 g (19.2 mmol, 86% purity) of the compound from Ex. 13A were dissolved in 181 ml of ethanol, and 11 ml of sodium ethoxide solution (21% by weight in ethanol) were added. Since the conversion was still incomplete after stirring at RT overnight, the mixture was stirred at 50° C. for a further 5 h. The reaction mixture was then poured onto ice-water and adjusted to pH 5 with acetic acid. The precipitated solids were filtered off, washed to neutrality with water and suction-dried. 5.66 g (100% of theory, 92% purity) of the title compound were obtained, which was used for subsequent reactions without further purification.

LC/MS (Method 1, ESIpos): Rₜ=1.85 min, m/z=265 [M+H]⁺.

### Example 29A

### 5-Methyl-3-(spiro[3.3]hept-2-yl)thieno[2,3-d]pyrimidine-2,4(1H,3H)-dione

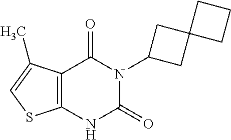 [SMILES: Cc1csc2[nH]c(=O)n(C3CC4(CCC4)C3)c(=O)c12]

Analogously to the process described in Ex. 16A, 5.32 g (16.5 mmol) of the compound from Ex. 14A were used to prepare 4.33 g (94% of theory) of the title compound.

¹H-NMR (400 MHz, DMSO-d₆, δ/ppm): 11.97 (s, 1H), 6.65 (d, 1H), 5.06 (quin, 1H), 2.94-2.81 (m, 2H), 2.33 (s, 3H), 2.20 (td, 2H), 2.10-2.02 (m, 2H), 2.01-1.93 (m, 2H), 1.86-1.75 (m, 2H).

LC/MS (Method 1, ESIpos): Rₜ=1.93 min, m/z=277.10 [M+H]⁺.

### Example 30A

### 3-Cyclopentyl-5-methylthieno[2,3-d]pyrimidine-2,4(1H,3H)-dione

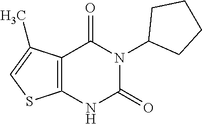 [SMILES: Cc1csc2[nH]c(=O)n(C3CCCC3)c(=O)c12]

5.96 g (20.1 mmol) of the compound from Ex. 15A were dissolved in 60 ml of ethanol, and 15 ml (40.2 mmol) of a 21% solution of sodium ethoxide in ethanol were added. The reaction mixture was stirred at 50° C. for about 16 h. This was followed by acidification by adding 1 M hydrochloric acid. In the course of this, the product precipitated out. The product was filtered off with suction, washed to neutrality with water and dried under high vacuum. 4.86 g (96% of theory) of the title compound were obtained.

¹H-NMR (400 MHz, DMSO-d₆, δ/ppm): 12.01 (s, 1H), 6.66 (d, 1H), 5.25 (quin, 1H), 2.34 (d, 3H), 2.12-1.98 (m, 2H), 1.95-1.81 (m, 2H), 1.79-1.65 (m, 2H), 1.61-1.47 (m, 2H).

LC/MS (Method 2, ESIpos): Rₜ=0.87 min, m/z=251 [M+H]⁺.

### Example 31A

### 3-Cyclopropyl-5-methyl-2,4-dioxo-1,2,3,4-tetrahydrothieno[2,3-d]pyrimidine-6-carbaldehyde

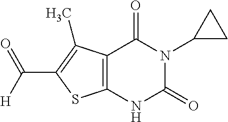 [SMILES: Cc1c(C=O)sc2[nH]c(=O)n(C3CC3)c(=O)c12]

To a solution of 10.95 g (49.3 mmol) of the compound from Ex. 16A in 37.9 ml (493 mmol) of DMF were cautiously added 55.1 ml (591 mmol) of phosphorus oxychloride. After the strongly exothermic reaction had abated, the mixture was stirred for another 15 min. Then the reaction mixture was stirred cautiously into 1.5 litres of water. After stirring at RT for about 15 h, the precipitated product was filtered off with suction, washed to neutrality with water and dried under high vacuum. 12.32 g (99% of theory) of the title compound were obtained.

¹H-NMR (400 MHz, DMSO-d₆, δ/ppm): 12.41 (s, 1H), 10.05 (s, 1H), 2.74 (s, 3H), 2.58-2.52 (m, 1H, partially concealed by DMSO signal), 1.08-0.91 (m, 2H), 0.78-0.62 (m, 2H).

LC/MS (Method 1, ESIpos): Rₜ=1.04 min, m/z=251.05 [M+H]⁺.

### Example 32A

### 5-Methyl-3-(1-methylcyclopropyl)-2,4-dioxo-1,2,3,4-tetrahydrothieno[2,3-d]pyrimidine-6-carbaldehyde

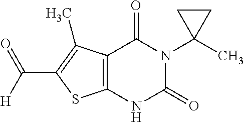 [SMILES: Cc1c(C=O)sc2[nH]c(=O)n(C3(C)CC3)c(=O)c12]

To a solution of 10.0 g (42.3 mmol) of the compound from Ex. 17A in 32.6 ml (423 mmol) of DMF were cautiously added 47.3 ml (508 mmol) of phosphorus oxychloride. After the strongly exothermic reaction had abated, the mixture was stirred for another 15 min. Then it was stirred cautiously into 1.5 litres of water. After stirring at RT for about 15 h, the precipitated product was filtered off with suction, washed to neutrality with water and dried under high vacuum. 10.50 g (94% of theory) of the title compound were obtained.

¹H-NMR (400 MHz, DMSO-d₆, δ/ppm): 12.40 (s, 1H), 10.05 (s, 1H), 2.75 (s, 3H), 1.33 (s, 3H), 0.91-0.82 (m, 4H).

LC/MS (Method 1, ESIpos): Rₜ=1.21 min, m/z=265.06 [M+H]⁺.

### Example 33A

### 5-Methyl-2,4-dioxo-3-[1-(trifluoromethyl)cyclopropyl]-1,2,3,4-tetrahydrothieno[2,3-d]pyrimidine-6-carbaldehyde

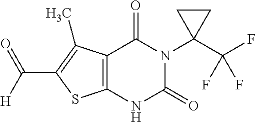 [SMILES: Cc1c(C=O)sc2[nH]c(=O)n(C3(C(F)(F)F)CC3)c(=O)c12]

Analogously to the process described in Ex. 31A, 1.66 g (5.72 mmol) of the compound from Ex. 18A, 6.4 ml (68.6 mmol) of phosphorus oxychloride and 4.4 ml (57.2 mmol) of DMF were used to prepare 1.80 g (99% of theory) of the title compound. The product here was extracted by stirring with water for only 1 h.

¹H-NMR (400 MHz, DMSO-d₆, δ/ppm): 12.66 (br. s, 1H), 10.07 (s, 1H), 2.75 (s, 3H), 1.68-1.46 (m, 2H), 1.42-1.27 (m, 2H).

LC/MS (Method 2, ESIpos): Rₜ=0.76 min, m/z=319 [M+H]⁺.

### Example 34A

### 5-Methyl-3-(2-methylcyclopropyl)-2,4-dioxo-1,2,3,4-tetrahydrothieno[2,3-d]pyrimidine-6-carbaldehyde (trans racemate)

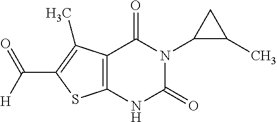 [SMILES: Cc1c(C=O)sc2[nH]c(=O)n(C3CC3C)c(=O)c12]

To a solution of 1.67 g (7.09 mmol) of the compound from Ex. 19A in 67 ml of DMF were cautiously added, while cooling with an ice bath, 6.6 ml (70.9 mmol) of phosphorus oxychloride.

The mixture was stirred at 70° C. for 1 h and then very substantially concentrated on a rotary evaporator. The residue obtained was added to ice-water and stirred. The precipitated product was filtered off with suction, washed to neutrality with water and dried. 1.46 g (72% of theory) of the title compound were obtained.

¹H-NMR (400 MHz, DMSO-d₆, δ/ppm): 12.42 (br. s, 1H), 10.04 (s, 1H), 2.73 (s, 3H), 2.19 (dt, 1H), 1.14 (d, 3H), 1.05-0.94 (m, 1H), 0.89-0.77 (m, 2H).

LC/MS (Method 4, ESIpos): Rₜ=0.88 min, m/z=265 [M+H]⁺.

### Example 35A

### 3-(2,2-Dimethylcyclopropyl)-5-methyl-2,4-dioxo-1,2,3,4-tetrahydrothieno[2,3-d]pyrimidine-6-carbaldehyde (racemate)

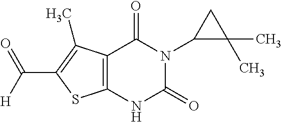 [SMILES: Cc1c(C=O)sc2[nH]c(=O)n(C3CC3(C)C)c(=O)c12]

To a solution of 1.64 g (6.4 mmol) of the compound from Ex. 20A in 60 ml of DMF were cautiously added, while cooling with an ice bath, 6 ml (64.1 mmol) of phosphorus oxychloride. The mixture was stirred at 70° C. for 1 h and then very substantially concentrated on a rotary evaporator. The residue obtained was added to ice-water and stirred. The precipitated product was filtered off with suction, washed to neutrality with water and dried. 1.57 g (88% of theory) of the title compound were obtained.

¹H-NMR (400 MHz, DMSO-d₆, δ/ppm): 12.45 (br. s, 1H), 10.05 (s, 1H), 2.74 (s, 3H), 2.34 (dd, 1H), 1.16 (s, 3H), 1.03 (dd, 1H), 0.88 (s, 3H), 0.79-0.69 (m, 1H).

LC/MS (Method 4, ESIpos): Rₜ=0.97 min, m/z=279 [M+H]⁺.

### Example 36A

### 3-(1-Ethylcyclopropyl)-5-methyl-2,4-dioxo-1,2,3,4-tetrahydrothieno[2,3-d]pyrimidine-6-carbaldehyde

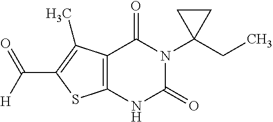 [SMILES: CCC1(n2c(=O)[nH]c3sc(C=O)c(C)c3c2=O)CC1]

Analogously to the process described in Ex. 31A, 1.26 g (5.03 mmol) of the compound from Ex. 21A, 4.7 ml (50.3 mmol) of phosphorus oxychloride and 4.7 ml (60.4 mmol) of DMF were used to prepare 1.30 g (92% of theory) of the title compound. The product here was extracted by stirring with water for only 2 h.

¹H-NMR (400 MHz, DMSO-d₆, δ/ppm): 12.38 (br. s, 1H), 10.04 (s, 1H), 2.74 (s, 3H), 1.74-1.62 (m, 2H), 1.01-0.75 (m, 4H), 0.82 (t, 3H).

LC/MS (Method 1, ESIpos): Rₜ=1.39 min, m/z=279.08 [M+H]⁺.

### Example 37A

### 3-Cyclobutyl-5-methyl-2,4-dioxo-1,2,3,4-tetrahydrothieno[2,3-d]pyrimidine-6-carbaldehyde

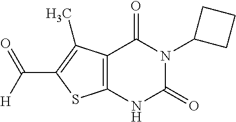 [SMILES: Cc1c(C=O)sc2[nH]c(=O)n(C3CCC3)c(=O)c12]

To a solution of 6.40 g (27.1 mmol) of the compound from Ex. 22A in 20.8 ml (271 mmol) of DMF were cautiously added 30.3 ml (325 mmol) of phosphorus oxychloride. After the strongly exothermic reaction had abated, the mixture was stirred for another 15 min. Then the reaction mixture was stirred cautiously into 1 litre of water. After stirring at RT for about 1 h, the precipitated product was filtered off with suction, washed to neutrality with water and dried under high vacuum. 7.11 g (99% of theory) of the title compound were obtained.

¹H-NMR (400 MHz, DMSO-d₆, δ/ppm): 12.47 (s, 1H), 10.05 (s, 1H), 5.16 (quin, 1H), 2.91-2.78 (m, 2H), 2.74 (s, 3H), 2.15 (qt, 2H), 1.90-1.62 (m, 2H).

LC/MS (Method 2, ESIpos): Rₜ=0.79 min, m/z=265 [M+H]⁺.

### Example 38A

### 3-(3,3-Difluorocyclobutyl)-5-methyl-2,4-dioxo-1,2,3,4-tetrahydrothieno[2,3-d]pyrimidine-6-carbaldehyde

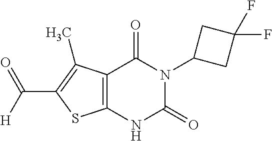 [SMILES: Cc1c(C=O)sc2[nH]c(=O)n(C3CC(F)(F)C3)c(=O)c12]

To a solution of 3.92 g (14.4 mmol) of the compound from Ex. 23A in 11 ml of DMF were added 12.7 ml (137 mmol) of phosphorus oxychloride. After the strongly exothermic reaction had abated, the mixture was stirred without further supply of heat for another 45 min. Then the reaction mixture was stirred cautiously into 1200 ml of ice-cold water. After stirring for 16 h, the precipitated product was filtered off with suction, washed with water until neutral and dried. 4.20 g (97% of theory) of the title compound were obtained.

LC/MS (Method 1, ESIpos): Rₜ=1.46 min, m/z=301 [M+H]⁺.

### Example 39A

### 5-Methyl-3-(1-methylcyclobutyl)-2,4-dioxo-1,2,3,4-tetrahydrothieno[2,3-d]pyrimidine-6-carbaldehyde

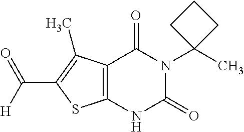 [SMILES: Cc1c(C=O)sc2[nH]c(=O)n(C3(C)CCC3)c(=O)c12]

Analogously to the process described in Ex. 37A, 5.56 g (22.2 mmol) of the compound from Ex. 25A, 24.8 ml (267 mmol) of phosphorus oxychloride and 17.1 ml (222 mmol) of DMF were used to prepare 5.96 g (96% of theory) of the title compound.

¹H-NMR (400 MHz, DMSO-d₆, δ/ppm): 12.34 (s, 1H), 10.05 (s, 1H), 2.72 (s, 3H), 2.42-2.18 (m, 4H), 1.82-1.58 (m, 2H), 1.51 (s, 3H).

LC/MS (Method 2, ESIpos): Rₜ=0.83 min, m/z=279 [M+H]⁺.

### Example 40A

### 3-(trans-3-Methoxycyclobutyl)-5-methyl-2,4-dioxo-1,2,3,4-tetrahydrothieno[2,3-d]pyrimidine-6-carbaldehyde

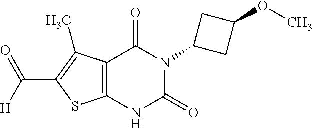 [SMILES: CO[C@H]1C[C@H](n2c(=O)[nH]c3sc(C=O)c(C)c3c2=O)C1]

To a solution of 4.43 g (16.6 mmol) of the compound from Ex. 26A in 13 ml of DMF were added 15 ml (160 mmol) of phosphorus oxychloride. After the strongly exothermic reaction had abated, the mixture was stirred without further supply of heat for another 1 h. Then the reaction mixture was stirred cautiously into 1200 ml of ice-cold water. After stirring for 16 h, the precipitated product was filtered off with suction, washed with water until neutral and dried. 4.47 g (85% of theory, 94% purity) of the title compound were obtained, which was used for subsequent reactions without further purification.

¹H-NMR (400 MHz, DMSO-d₆, δ/ppm): 12.53 (s, 1H), 10.06 (s, 1H), 5.48-5.37 (m, 1H), 4.13-4.10 (m, 1H), 3.16 (s, 3H), 2.96-2.89 (m, 2H), 2.75 (s, 3H), 2.25-2.19 (m, 2H).

LC/MS (Method 2, ESIpos): Rₜ=0.69 min, m/z=295 [M+H]⁺.

### Example 41A

### 3-(cis-3-Methoxycyclobutyl)-5-methyl-2,4-dioxo-1,2,3,4-tetrahydrothieno[2,3-d]pyrimidine-6-carbaldehyde

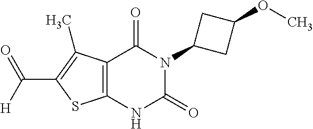 [SMILES: CO[C@H]1C[C@@H](n2c(=O)[nH]c3sc(C=O)c(C)c3c2=O)C1]

To a solution of 4.66 g (17.1 mmol) of the compound from Ex. 27A in 13 ml of DMF were added 15 ml (162 mmol) of phosphorus oxychloride. After the strongly exothermic reaction had abated, the mixture was stirred without further supply of heat for another 30 min. Then the reaction mixture was stirred cautiously into 1.7 litres of ice-cold water. After stirring for 16 h, the precipitated product was filtered off with suction, washed with water until neutral and dried. 3.99 g (60% of theory, 76% purity) of the title compound were obtained, which was used for subsequent reactions without further purification.

LC/MS (Method 1, ESIpos): Rₜ=1.21 min, m/z=295 [M+H]⁺.

### Example 42A

### 3-(3,3-Dimethylcyclobutyl)-5-methyl-2,4-dioxo-1,2,3,4-tetrahydrothieno[2,3-d]pyrimidine-6-carbaldehyde

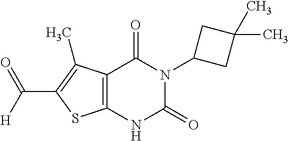 [SMILES: Cc1c(C=O)sc2[nH]c(=O)n(C3CC(C)(C)C3)c(=O)c12]

To a solution of 5.50 g (19.0 mmol, 92% purity) of the compound from Ex. 28A in 15 ml of DMF were added 17 ml (181 mmol) of phosphorus oxychloride. After the strongly exothermic reaction had abated, the mixture was stirred without further supply of heat for another 30 min. Then the reaction mixture was stirred cautiously into 2 litres of ice-cold water. After stirring for 16 h, the precipitated product was filtered off with suction, washed with water until neutral and dried. 4.80 g (87% of theory) of the title compound were obtained, which was used for subsequent reactions without further purification.

LC/MS (Method 1, ESIpos): Rₜ=1.80 min, m/z=293 [M+H]⁺.

### Example 43A

### 5-Methyl-2,4-dioxo-3-(spiro[3.3]hept-2-yl)-1,2,3,4-tetrahydrothieno[2,3-d]pyrimidine-6-carbaldehyde

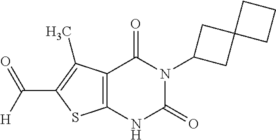 [SMILES: Cc1c(C=O)sc2[nH]c(=O)n(C3CC4(CCC4)C3)c(=O)c12]

Analogously to the process described in Ex. 37A, 4.32 g (15.6 mmol) of the compound from Ex. 29A, 14.5 ml (188 mmol) of phosphorus oxychloride and 12 ml (156 mmol) of DMF were used to prepare 4.73 g (99% of theory) of the title compound.

¹H-NMR (400 MHz, DMSO-d₆, δ/ppm): 12.45 (br. s, 1H), 10.04 (s, 1H), 5.07-4.92 (m, 1H), 2.88-2.78 (m, 2H), 2.73 (s, 3H), 2.28-2.18 (m, 2H), 2.11-2.02 (m, 2H), 2.02-1.93 (m, 2H), 1.88-1.74 (m, 2H).

LC/MS (Method 2, ESIpos): Rₜ=1.00 min, m/z=305 [M+H]⁺.

### Example 44A

### 3-Cyclopentyl-5-methyl-2,4-dioxo-1,2,3,4-tetrahydrothieno[2,3-d]pyrimidine-6-carbaldehyde

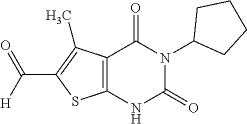 [SMILES: Cc1c(C=O)sc2[nH]c(=O)n(C3CCCC3)c(=O)c12]

To a solution of 4.85 g (19.4 mmol) of the compound from Ex. 30A in 14.9 ml (194 mmol) of DMF were cautiously added 21.7 ml (233 mmol) of phosphorus oxychloride. After the strongly exothermic reaction had abated, the mixture was stirred for another 15 min. Then the reaction mixture was stirred cautiously into 1.5 litre of water. After stirring at RT for about 1 h, the precipitated product was filtered off with suction, washed to neutrality with water and dried under high vacuum. 5.11 g (91% of theory, 97% purity) of the title compound were obtained.

¹H-NMR (400 MHz, DMSO-d₆, δ/ppm): 12.49 (s, 1H), 10.06 (s, 1H), 5.23 (quin, 1H), 2.75 (s, 3H), 2.11-1.96 (m, 2H), 1.95-1.82 (m, 2H), 1.81-1.68 (m, 2H), 1.62-1.47 (m, 2H).

LC/MS (Method 6, ESIpos): Rₜ=1.14 min, m/z=279 [M+H]⁺.

### Example 45A

### 1-(2-Methoxyethyl)-5-methyl-3-(1-methylcyclopropyl)thieno[2,3-d]pyrimidine-2,4(1H,3H)-dione

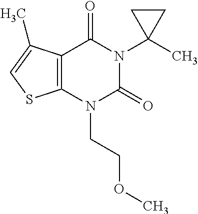 [SMILES: COCCn1c(=O)n(C2(C)CC2)c(=O)c2c(C)csc21]

33.0 g (140 mmol) of the compound from Ex. 17A and 48.25 g (349 mmol) of potassium carbonate were stirred in 400 ml of anhydrous DMF at RT for 30 min, before 26.3 ml (279 mmol) of 2-bromoethyl methyl ether were added. Then the reaction mixture was stirred at RT for about 16 h. Subsequently, water was added and the mixture was stirred at RT for 30 min. The product which precipitated out was filtered off with suction, washed with water and dried under high vacuum. 33.54 g (81% of theory) of the title compound were obtained.

¹H-NMR (400 MHz, DMSO-d₆, δ/ppm): 6.76 (d, 1H), 4.20-3.81 (m, 2H), 3.62 (t, 2H), 3.24 (s, 3H), 2.36 (d, 3H), 1.34 (s, 3H), 1.01-0.72 (m, 4H).

LC/MS (Method 1, ESIpos): Rₜ=1.62 min, m/z=295.11 [M+H]⁺.

### Example 46A

### 5-Methyl-3-(oxetan-3-yl)-1-(3,3,3-trifluoropropyl)thieno[2,3-d]pyrimidine-2,4(1H,3H)-dione

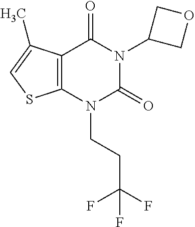 [SMILES: Cc1csc2c1c(=O)n(C1COC1)c(=O)n2CCC(F)(F)F]

Analogously to the process described in Ex. 45A, 1.42 g (5.96 mmol) of the compound from Ex. 24A and 4.0 g (17.9 mmol) of 1,1,1-trifluoro-3-iodopropane were used to prepare 1.75 g (87% of theory) of the title compound.

¹H-NMR (400 MHz, DMSO-d₆, δ/ppm): 6.89 (d, 1H), 5.04 (quin, 1H), 4.79-4.64 (m, 4H), 4.08 (t, 2H), 2.75 (qt, 2H), 2.35 (d, 3H).

LC/MS (Method 1, ESIpos): Rₜ=1.63 min, m/z=335.07 [M+H]⁺.

### Example 47A

### 6-Bromo-5-methyl-3-(oxetan-3-yl)-1-(3,3,3-trifluoropropyl)thieno[2,3-d]pyrimidine-2,4(1H,3H)-dione

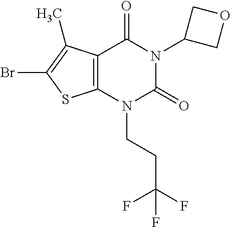 [SMILES: Cc1c(Br)sc2c1c(=O)n(C1COC1)c(=O)n2CCC(F)(F)F]

To a solution of 1.75 g (5.24 mmol) of the compound from Ex. 46A in 30 ml of dichloromethane were added, at 0° C., 978 mg (5.50 mmol) of N-bromosuccinimide (NBS). The cooling bath was removed, and the reaction mixture was stirred at RT for 1 h. Then it was concentrated to dryness on a rotary evaporator. The remaining residue was taken up in ethyl acetate and washed successively with water and saturated sodium chloride solution. After drying over anhydrous magnesium sulfate, the mixture was filtered and concentrated again. 2.17 g (96% of theory, 96% purity) of the title compound were obtained.

¹H-NMR (400 MHz, DMSO-d₆, δ/ppm): 5.04 (quin, 1H), 4.83-4.58 (m, 4H), 4.05 (t, 2H), 2.75 (qt, 2H), 2.32 (s, 3H).

LC/MS (Method 1, ESIpos): Rₜ=1.99 min, m/z=412.98/414.98 [M+H]⁺.

### Example 48A

### 3-Cyclopropyl-1,5-dimethyl-2,4-dioxo-1,2,3,4-tetrahydrothieno[2,3-d]pyrimidine-6-carbaldehyde

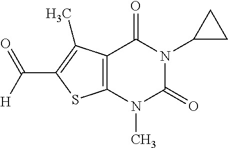 [SMILES: Cc1c(C=O)sc2c1c(=O)n(C1CC1)c(=O)n2C]

500 mg (2.0 mmol) of the compound from Ex. 31A and 976 mg (3.0 mmol) of caesium carbonate were stirred in 10 ml of anhydrous DMF at RT for 10 min, before 284 μl (3.0 mmol) of dimethyl sulfate were added. Then the reaction mixture was heated in a microwave oven (Biotage Initiator with dynamic control of irradiation power) to 60° C. for 1 h. After cooling to RT, water was added and the reaction mixture was stirred at RT for 30 min. The product which precipitated out was filtered off with suction, washed with water and dried under high vacuum. 440 mg (83% of theory) of the title compound were obtained.

¹H-NMR (400 MHz, DMSO-d₆, δ/ppm): 10.07 (s, 1H), 3.43 (s, 3H), 2.77 (s, 3H), 2.61 (tt, 1H), 1.10-0.94 (m, 2H), 0.78-0.63 (m, 2H).

LC/MS (Method 1, ESIpos): Rₜ=1.27 min, m/z=265.06 [M+H]⁺.

### Example 49A

### 1-Butyl-3-cyclopropyl-5-methyl-2,4-dioxo-1,2,34-tetrahydrothieno[2,3-d]pyrimidine-6-carbaldehyde

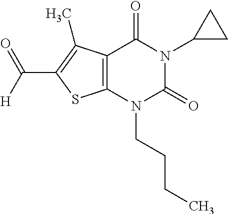 [SMILES: CCCCn1c(=O)n(C2CC2)c(=O)c2c(C)c(C=O)sc21]

Analogously to the method described in Ex. 45A, 500 mg (2.00 mmol) of the compound from Ex. 31A and 735 mg (4.00 mmol) of n-butyl iodide were used to prepare 515 mg (84% of theory) of the title compound.

¹H-NMR (400 MHz, DMSO-d₆, δ/ppm): 10.07 (s, 1H), 3.89 (t, 2H), 2.77 (s, 3H), 2.66-2.57 (m, 1H), 1.65 (quin, 2H), 1.35 (sext, 2H), 1.07-0.97 (m, 2H), 0.91 (t, 3H), 0.75-0.65 (m, 2H).

LC/MS (Method 1, ESIpos): Rₜ=1.83 min, m/z=307.11 [M+H]⁺.

### Example 50A

### 3-Cyclopropyl-1-(3-fluoropropyl)-5-methyl-2,4-dioxo-1,2,3,4-tetrahydrothieno[2,3-d]pyrimidine-6-carbaldehyde

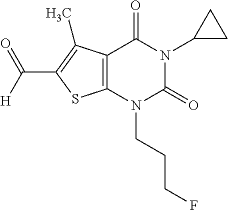 [SMILES: Cc1c(C=O)sc2c1c(=O)n(C1CC1)c(=O)n2CCCF]

To a solution of 390 mg (1.56 mmol) of the compound from Ex. 31A in 16.2 ml of DMF were added 538 mg (3.89 mmol) of potassium carbonate, and the mixture was stirred at RT for 15 min. Then 879 mg (4.67 mmol) of 1-fluoro-3-iodopropane were added, and the mixture was stirred at 50° C. for 17 h. The DMF was very substantially distilled off and the residue obtained was partitioned between semisaturated sodium chloride solution (100 ml) and ethyl acetate (50 ml). The water phase was extracted with ethyl acetate. The combined organic phases were dried over sodium sulfate, filtered and concentrated. The residue obtained was chromatographed using a silica gel cartridge (Biotage, 50 g of silica gel, eluent: hexane/ethyl acetate 92:8→34:66). 391 mg (80% of theory) of the title compound were obtained.

¹H-NMR (400 MHz, DMSO-d₆, δ/ppm): 10.08 (s, 1H), 4.60 (t, 1H), 4.48 (t, 1H), 4.00 (t, 2H), 2.77 (s, 3H), 2.65-2.56 (m, 1H), 2.14-2.06 (m, 1H), 2.06-1.99 (m, 1H), 1.05-0.98 (m, 2H), 0.73-0.66 (m, 2H).

LC/MS (Method 4, ESIpos): Rₜ=0.95 min, m/z=311 [M+H]⁺.

### Example 51A

### 3-Cyclopropyl-5-methyl-2,4-dioxo-1-(3,3,3-trifluoropropyl)-1,2,3,4-tetrahydrothieno[2,3-d]pyrimidine-6-carbaldehyde

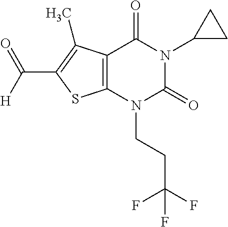 [SMILES: Cc1c(C=O)sc2c1c(=O)n(C1CC1)c(=O)n2CCC(F)(F)F]

900 mg (3.60 mmol) of the compound from Ex. 31A and 1.24 g (9.0 mmol) of potassium carbonate were stirred in 15 ml of anhydrous DMF at RT for 30 min, before 1.3 ml (10.8 mmol) of 1,1,1-trifluoro-3-iodopropane were added. Then the reaction mixture was stirred first at RT for 2 h and then at 50° C. for about 16 h. After cooling to RT, water was added and the mixture was extracted with ethyl acetate. The organic extract was concentrated and the residue was purified by means of MPLC (Biotage Isolera One, SNAP KP-Sil cartridge, 10 g of silica gel, eluent: cyclohexane/ethyl acetate 2:1). After concentration of the product fractions and drying under high vacuum, 1.07 g (85% of theory) of the title compound were obtained.

¹H-NMR (400 MHz, DMSO-d₆, δ/ppm): 10.09 (s, 1H), 4.13 (t, 2H), 2.87-2.69 (m, 2H), 2.78 (s, 3H), 2.63 (tt, 1H), 1.09-0.96 (m, 2H), 0.75-0.65 (m, 2H).

LC/MS (Method 2, ESIpos): Rₜ=0.89 min, m/z=347 [M+H]⁺.

### Example 52A

### 1-(Cyclobutylmethyl)-3-cyclopropyl-5-methyl-2,4-dioxo-1,2,3,4-tetrahydrothieno[2,3-d]pyrimidine-6-carbaldehyde

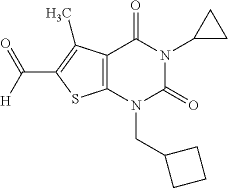 [SMILES: Cc1c(C=O)sc2c1c(=O)n(C1CC1)c(=O)n2CC1CCC1]

500 mg (2.00 mmol) of the compound from Ex. 31A and 690 mg (5.00 mmol) of potassium carbonate were stirred in 10 ml of anhydrous DMF at RT for 15 min, before 449 μl (4.00 mmol) of (bromomethyl)cyclobutane were added. Then the reaction mixture was stirred at 50° C. for about 16 h. After cooling to RT, water was added and the mixture was extracted with ethyl acetate. The organic extract was washed with saturated sodium chloride solution, dried over anhydrous magnesium sulfate, filtered and concentrated. The product was isolated by means of MPLC (Biotage Isolera One, SNAP KP-Sil cartridge, 50 g of silica gel, eluent: cyclohexane/ethyl acetate 2:1). After concentration of the product fractions and drying under high vacuum, 486 mg (76% of theory) of the title compound were obtained.

¹H-NMR (400 MHz, DMSO-d₆, δ/ppm): 10.07 (s, 1H), 3.96 (d, 2H), 2.82-2.69 (m, 1H), 2.76 (s, 3H), 2.66-2.58 (m, 1H), 2.04-1.91 (m, 2H), 1.89-1.74 (m, 4H), 1.07-0.96 (m, 2H), 0.73-0.64 (m, 2H).

LC/MS (Method 1, ESIpos): Rₜ=1.89 min, m/z=319.11 [M+H]⁺.

### Example 53A

### 3-Cyclopropyl-1-[(2,2-difluorocyclopropyl)methyl]-5-methyl-2,4-dioxo-1,2,3,4-tetrahydrothieno[2,3-d]pyrimidine-6-carbaldehyde (racemate)

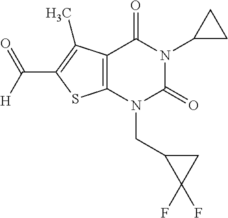 [SMILES: Cc1c(C=O)sc2c1c(=O)n(C1CC1)c(=O)n2CC1CC1(F)F]

Analogously to the method described in Ex. 52A, 1.0 g (4.00 mmol) of the compound from Ex. 31A and 1.02 g (6.00 mmol) of racemic 2-(bromomethyl)-1,1-difluorocyclopropane were used to prepare 980 mg (72% of theory) of the title compound. The reaction in this case was not effected at 50° C., but at RT.

¹H-NMR (400 MHz, DMSO-d₆, δ/ppm): 10.09 (s, 1H), 4.17 (ddd, 1H), 3.95 (dd, 1H), 2.78 (s, 3H), 2.67-2.58 (m, 1H), 2.30-2.12 (m, 1H), 1.78-1.62 (m, 1H), 1.49 (dtd, 1H), 1.10-0.94 (m, 2H), 0.79-0.64 (m, 2H).

LC/MS (Method 1, ESIpos): Rₜ=1.68 min, m/z=341.08 [M+H]⁺.

### Example 54A

### (3-Cyclopropyl-6-formyl-5-methyl-2,4-dioxo-3,4-dihydrothieno[2,3-d]pyrimidin-1(2H)-yl)acetonitrile

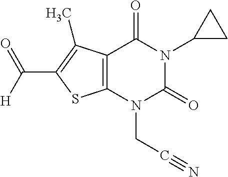 [SMILES: Cc1c(C=O)sc2c1c(=O)n(C1CC1)c(=O)n2CC#N]

Analogously to the method described in Ex. 52A, 500 mg (2.00 mmol) of the compound from Ex. 31A and 479 mg (4.00 mmol) of bromoacetonitrile were used to prepare 240 mg (40% of theory) of the title compound. The reaction in this case was not effected at 50° C., but at RT.

¹H-NMR (400 MHz, DMSO-d₆, δ/ppm): 10.10 (s, 1H), 5.18 (s, 2H), 2.78 (s, 3H), 2.69-2.59 (m, 1H), 1.12-0.94 (m, 2H), 0.81-0.63 (m, 2H).

LC/MS (Method 1, ESIpos): Rₜ=1.30 min, m/z=290.06 [M+H]⁺.

### Example 55A

### 3-Cyclopropyl-1-(2-methoxyethyl)-5-methyl-2,4-dioxo-1,2,3,4-tetrahydrothieno[2,3-d]pyrimidine-6-carbaldehyde

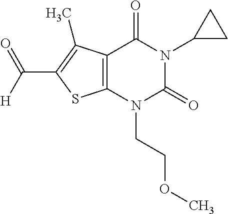 [SMILES: COCCn1c(=O)n(C2CC2)c(=O)c2c(C)c(C=O)sc21]

1.0 g (4.00 mmol) of the compound from Ex. 31A and 1.38 g (10.0 mmol) of potassium carbonate were stirred in 35 ml of anhydrous DMF at RT for 15 min, before 751 μl (8.00 mmol) of 2-bromoethyl methyl ether were added. Then the reaction mixture was stirred at 50° C. for about 16 h. After cooling to RT, water was added and the mixture was extracted with ethyl acetate. The organic extract was washed successively with water and saturated sodium chloride solution, dried over anhydrous magnesium sulfate, filtered and concentrated. The product was isolated by means of MPLC (Biotage Isolera One, SNAP KP-Sil cartridge, 100 g of silica gel, eluent: cyclohexane/ethyl acetate 2:1). After concentration of the product fractions and drying under high vacuum, a first fraction of 942 mg of the title compound was obtained. A likewise obtained mixed fraction was concentrated, and the residue was purified by means of preparative HPLC (Method 11). After concentration of the product fractions and drying under high vacuum, this gave a second fraction of 120 mg of the title compound. A total of 1.06 g (86% of theory) of the title compound was thus obtained.

¹H-NMR (500 MHz, DMSO-d₆, δ/ppm): 10.07 (s, 1H), 4.06 (t, 2H), 3.64 (t, 2H), 3.24 (s, 3H), 2.76 (s, 3H), 2.67-2.58 (m, 1H), 1.07-0.97 (m, 2H), 0.75-0.68 (m, 2H).

LC/MS (Method 1, ESIpos): Rₜ=1.36 min, m/z=309.09 [M+H]⁺.

### Example 56A

### 3-Cyclopropyl-1-(2-ethoxyethyl)-5-methyl-2,4-dioxo-1,2,3,4-tetrahydrothieno[2,3-d]pyrimidine-6-carbaldehyde

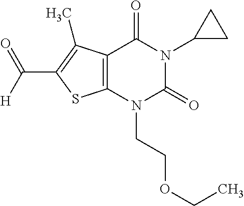 [SMILES: CCOCCn1c(=O)n(C2CC2)c(=O)c2c(C)c(C=O)sc21]

500 mg (2.00 mmol) of the compound from Ex. 31A and 690 mg (5.00 mmol) of potassium carbonate were stirred in 10 ml of anhydrous DMF at RT for 15 min, before 611 mg (4.00 mmol) of 2-bromoethyl ethyl ether were added. Then the reaction mixture was stirred at 50° C. for about 16 h. Subsequently, water was added and the mixture was stirred at RT for 30 min. The product which precipitated out was filtered off with suction, washed with water and dried under high vacuum. This gave a first fraction of 222 mg of the title compound. The filtrate was extracted with ethyl acetate. The organic extract was washed successively with water and saturated sodium chloride solution, dried over anhydrous magnesium sulfate, filtered and concentrated. A second fraction of 232 mg of the title compound was isolated from this residue by means of MPLC (Biotage Isolera One, SNAP KP-Sil cartridge, 50 g of silica gel, eluent: cyclohexane/ethyl acetate 2:1). A total of 454 mg (70% of theory) of the title compound was thus obtained.

¹H-NMR (400 MHz, DMSO-d₆, δ/ppm): 10.07 (s, 1H), 4.05 (t, 2H), 3.67 (t, 2H), 3.44 (q, 2H), 2.76 (s, 3H), 2.62 (tt, 1H), 1.09-0.96 (m, 2H), 1.03 (t, 3H), 0.76-0.66 (m, 2H).

LC/MS (Method 1, ESIpos): Rₜ=1.55 min, m/z=232.11 [M+H]⁺.

### Example 57A

### 3-Cyclopropyl-1-(2-isopropoxyethyl)-5-methyl-2,4-dioxo-1,2,3,4-tetrahydrothieno[2,3-d]pyrimidine-6-carbaldehyde

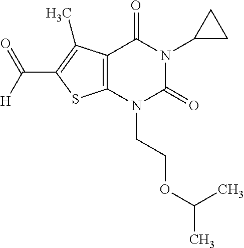 [SMILES: Cc1c(C=O)sc2c1c(=O)n(C1CC1)c(=O)n2CCOC(C)C]

Analogously to the process described in Ex. 52A, 500 mg (2.00 mmol) of the compound from Ex. 31A and 667 mg (4.00 mmol) of 2-bromoethyl isopropyl ether were used to obtain 513 mg (74% of theory) of the title compound.

¹H-NMR (400 MHz, DMSO-d₆, δ/ppm): 10.07 (s, 1H), 4.02 (t, 2H), 3.66 (t, 2H), 3.56 (sept, 1H), 2.76 (s, 3H), 2.63 (tt, 1H), 1.07-0.97 (m, 2H), 1.01 (d, 6H), 0.74-0.65 (m, 2H).

LC/MS (Method 1, ESIpos): Rₜ=1.71 min, m/z=337.12 [M+H]⁺.

### Example 58A

### 3-Cyclopropyl-5-methyl-2,4-dioxo-1-[2-(trifluoromethoxy)ethyl]-1,2,3,4-tetrahydrothieno[2,3-d]pyrimidine-6-carbaldehyde

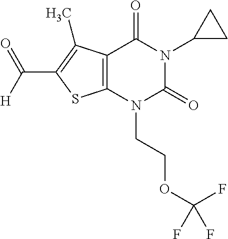 [SMILES: Cc1c(C=O)sc2c1c(=O)n(C1CC1)c(=O)n2CCOC(F)(F)F]

500 mg (2.00 mmol) of the compound from Ex. 31A and 690 mg (5.00 mmol) of potassium carbonate were stirred in 10 ml of anhydrous DMF at RT for 15 min, before 771 mg (4.00 mmol) of 1-bromo-2-(trifluoromethoxy)ethane [commercially available; lit.: P. E. Aldrich, W. A. Sheppard, J. Org. Chem. 29 (1), 11-15 (1964)] were added. Then the reaction mixture was stirred first at RT for about 16 h and then at 50° C. for about 24 h. After cooling to RT, water was added and the mixture was extracted with ethyl acetate. The organic extract was washed successively with water and saturated sodium chloride solution, dried over anhydrous magnesium sulfate, filtered and concentrated. After the residue had been dried under high vacuum, 592 mg (81% of theory) of the title compound were obtained.

¹H-NMR (400 MHz, DMSO-d₆, δ/ppm): 10.08 (s, 1H), 4.45-4.35 (m, 2H), 4.30-4.18 (m, 2H), 2.77 (s, 3H), 2.67-2.59 (m, 1H), 1.08-0.97 (m, 2H), 0.77-0.65 (m, 2H).

LC/MS (Method 2, ESIpos): Rₜ=0.90 min, m/z=363 [M+H]⁺.

### Example 59A

### 3-Cyclopropyl-5-methyl-2,4-dioxo-1-(tetrahydrofuran-2-ylmethyl)-1,2,3,4-tetrahydrothieno[2,3-d]pyrimidine-6-carbaldehyde (racemate)

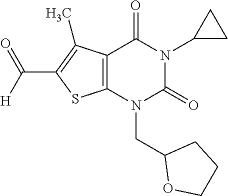 [SMILES: Cc1c(C=O)sc2c1c(=O)n(C1CC1)c(=O)n2CC1CCCO1]

628 mg (2.51 mmol) of the compound from Ex. 31A and 867 mg (6.27 mmol) of potassium carbonate were stirred in 23 ml of anhydrous DMF at RT for 15 min, before 570 μl (5.02 mmol) of racemic 2-(bromomethyl)tetrahydrofuran were added. Then the reaction mixture was stirred at 70° C. for about 16 h. After this time, the same amount of racemic 2-(bromomethyl)tetrahydrofuran again was added and the stirring was continued at 70° C. for 7 days. After cooling to RT, water was added to the reaction mixture, which was extracted with ethyl acetate. The organic extract was washed with saturated sodium chloride solution, dried over anhydrous magnesium sulfate, filtered and concentrated. The product was isolated by means of MPLC (Biotage Isolera One, SNAP KP-Sil cartridge, 100 g of silica gel, eluent: cyclohexane/ethyl acetate 1:1). After concentration of the product fractions and drying under high vacuum, 730 mg (84% of theory, 97% purity) of the title compound were obtained.

¹H-NMR (400 MHz, DMSO-d₆, δ/ppm): 10.07 (s, 1H), 4.26-4.17 (m, 1H), 4.10 (dd, 1H), 3.79-3.68 (m, 2H), 3.66-3.55 (m, 1H), 2.76 (s, 3H), 2.66-2.59 (m, 1H), 2.06-1.74 (m, 3H), 1.73-1.59 (m, 1H), 1.09-0.94 (m, 2H), 0.76-0.64 (m, 2H).

LC/MS (Method 1, ESIpos): Rₜ=1.48 min, m/z=335.11 [M+H]⁺.

### Example 60A

### 3-Cyclopropyl-5-methyl-2,4-dioxo-1-[(2R)-tetrahydrofuran-2-ylmethyl]-1,2,3,4-tetrahydrothieno[2,3-d]pyrimidine-6-carbaldehyde

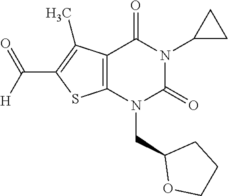 [SMILES: Cc1c(C=O)sc2c1c(=O)n(C1CC1)c(=O)n2C[C@H]1CCCO1]

Analogously to the method described in Ex. 59A, 500 mg (2.00 mmol) of the compound from Ex. 31A and 989 mg (6.00 mmol) of (2R)-2-(bromomethyl)tetrahydrofuran were used to prepare 382 mg (56% of theory) of the title compound.

¹H-NMR (400 MHz, DMSO-d₆, δ/ppm): 10.07 (s, 1H), 4.26-4.16 (m, 1H), 4.10 (dd, 1H), 3.79-3.68 (m, 2H), 3.66-3.57 (m, 1H), 2.76 (s, 3H), 2.67-2.58 (m, 1H), 2.06-1.75 (m, 3H), 1.72-1.60 (m, 1H), 1.07-0.97 (m, 2H), 0.75-0.65 (m, 2H).

LC/MS (Method 1, ESIpos): Rₜ=1.53 min, m/z=335.11 [M+H]⁺.

### Example 61A

### 3-Cyclopropyl-5-methyl-2,4-dioxo-l-[(2S)-tetrahydrofuran-2-ylmethyl]-1,2,3,4-tetrahydrothieno[2,3-d]pyrimidine-6-carbaldehyde

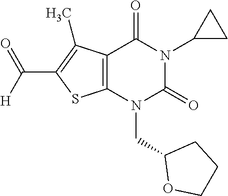 [SMILES: Cc1c(C=O)sc2c1c(=O)n(C1CC1)c(=O)n2C[C@@H]1CCCO1]

Analogously to the method described in Ex. 59A, 500 mg (2.00 mmol) of the compound from Ex. 31A and 989 mg (6.00 mmol) of (2S)-2-(bromomethyl)tetrahydrofuran were used to prepare 330 mg (48% of theory) of the title compound.

¹H-NMR (400 MHz, DMSO-d₆, δ/ppm): 10.07 (s, 1H), 4.26-4.16 (m, 1H), 4.10 (dd, 1H), 3.79-3.68 (m, 2H), 3.66-3.57 (m, 1H), 2.76 (s, 3H), 2.67-2.59 (m, 1H), 2.06-1.74 (m, 3H), 1.72-1.61 (m, 1H), 1.07-0.97 (m, 2H), 0.76-0.66 (m, 2H).

LC/MS (Method 1, ESIpos): Rₜ=1.53 min, m/z=335.11 [M+H]⁺.

### Example 62A

### 1,5-Dimethyl-3-(1-methylcyclopropyl)-2,4-dioxo-1,2,3,4-tetrahydrothieno[2,3-d]pyrimidine-6-carbaldehyde

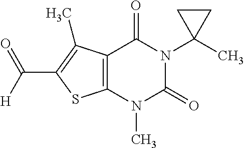 [SMILES: Cc1c(C=O)sc2c1c(=O)n(C1(C)CC1)c(=O)n2C]

Analogously to the method described in Ex. 48A, 500 mg (1.89 mmol) of the compound from Ex. 32A and 269 μl (2.84 mmol) of dimethyl sulfate were used to prepare 465 mg (85% of theory, 97% purity) of the title compound.

¹H-NMR (400 MHz, DMSO-d₆, δ/ppm): 10.07 (s, 1H), 3.43 (s, 3H), 2.78 (s, 3H), 1.34 (s, 3H), 1.01-0.76 (m, 4H).

LC/MS (Method 1, ESIpos): Rₜ=1.46 min, m/z=279.08 [M+H]⁺.

### Example 63A

### 1-Butyl-5-methyl-3-(1-methylcyclopropyl)-2,4-dioxo-1,2,3,4-tetrahydrothieno[2,3-d]pyrimidine-6-carbaldehyde

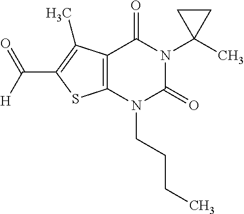 [SMILES: CCCCn1c(=O)n(C2(C)CC2)c(=O)c2c(C)c(C=O)sc21]

500 mg (1.89 mmol) of the compound from Ex. 32A and 654 mg (4.73 mmol) of potassium carbonate were stirred in 10 ml of anhydrous DMF at RT for 15 min, before 696 mg (3.78 mmol) of n-butyl iodide were added. Then the reaction mixture was stirred at RT for about 16 h. Subsequently, water was added and extraction was effected with ethyl acetate. The organic extract was washed successively with water and saturated sodium chloride solution, dried over anhydrous magnesium sulfate and concentrated. The residue was purified by means of MPLC (Biotage Isolera One, SNAP KP-Sil cartridge, 100 g of silica gel, eluent: cyclohexane/ethyl acetate 2:1). After concentration of the product fractions and drying under high vacuum, 557 mg (91% of theory) of the title compound were obtained.

¹H-NMR (400 MHz, DMSO-d₆, δ/ppm): 10.08 (s, 1H), 4.01-3.76 (m, 2H), 2.77 (s, 3H), 1.65 (quin, 2H), 1.43-1.28 (m, 2H), 1.34 (s, 3H), 0.99-0.77 (m, 4H), 0.92 (t, 3H).

LC/MS (Method 1, ESIpos): Rₜ=2.02 min, m/z=321.13 [M+H]⁺.

### Example 64A

### 5-Methyl-3-(1-methylcyclopropyl)-2,4-dioxo-1-(3,3,3-trifluoropropyl)-1,2,3,4-tetrahydrothieno[2,3-d]pyrimidine-6-carbaldehyde

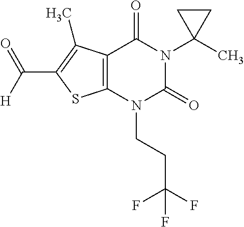 [SMILES: Cc1c(C=O)sc2c1c(=O)n(C1(C)CC1)c(=O)n2CCC(F)(F)F]

To a solution of 2.36 g (8.94 mmol) of the compound from Ex. 32A in 47 ml of DMF were added 3.10 g (22.4 mmol) of potassium carbonate, and the mixture was stirred at RT for 15 min. Then 3.1 ml (26.8 mmol) of 1,1,1-trifluoro-3-iodopropane were added. The mixture was stirred at 50° C. overnight. After cooling to RT, water was added to the mixture, which was extracted with dichloromethane. The organic phase was washed with saturated sodium chloride solution. After drying over anhydrous magnesium sulfate, the mixture was filtered and concentrated. 2.57 g (75% of theory, 94% purity) of the title compound were obtained.

LC/MS (Method 2, ESIpos): Rₜ=0.97 min, m/z=361 [M+H]⁺.

### Example 65A

### 1-(Cyclobutylmethyl)-5-methyl-3-(1-methylcyclopropyl)-2,4-dioxo-1,2,3,4-tetrahydrothieno[2,3-d]pyrimidine-6-carbaldehyde

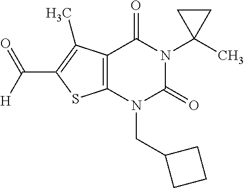 [SMILES: Cc1c(C=O)sc2c1c(=O)n(C1(C)CC1)c(=O)n2CC1CCC1]

500 mg (1.89 mmol) of the compound from Ex. 32A and 654 mg (4.73 mmol) of potassium carbonate were stirred in 10 ml of anhydrous DMF at RT for 15 min, before 564 mg (3.78 mmol) of (bromomethyl)cyclobutane were added. Then the reaction mixture was stirred first at RT for about 16 h and then at 50° C. for 8 h. Then water was added and extraction was effected with ethyl acetate. The organic extract was washed successively with water and saturated sodium chloride solution, dried over anhydrous magnesium sulfate and concentrated. The residue was purified by means of MPLC (Biotage Isolera One, SNAP KP-Sil cartridge, 100 g of silica gel, eluent: cyclohexane/ethyl acetate 2:1). After concentration of the product fractions and drying under high vacuum, 478 mg (76% of theory) of the title compound were obtained.

¹H-NMR (400 MHz, DMSO-d₆, δ/ppm): 10.07 (s, 1H), 4.14-3.78 (m, 2H), 2.84-2.69 (m, 1H), 2.77 (s, 3H), 2.05-1.90 (m, 2H), 1.89-1.73 (m, 4H), 1.34 (s, 3H), 0.99-0.72 (m, 4H).

LC/MS (Method 1, ESIpos): Rₜ=2.08 min, m/z=333.13 [M+H]⁺.

### Example 66A

### 1-[(2,2-Difluorocyclopropyl)methyl]-5-methyl-3-(1-methylcyclopropyl)-2,4-dioxo-1,2,3,4-tetrahydrothieno[2,3-d]pyrimidine-6-carbaldehyde (racemate)

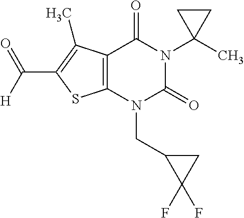 [SMILES: Cc1c(C=O)sc2c1c(=O)n(C1(C)CC1)c(=O)n2CC1CC1(F)F]

1.0 g (3.78 mmol) of the compound from Ex. 32A and 1.85 g (5.68 mmol) of caesium carbonate were stirred in 25 ml of anhydrous DMF at RT for 15 min, before 970 mg (5.68 mmol) of racemic 2-(bromomethyl)-1,1-difluorocyclopropane were added. Then the reaction mixture was stirred at RT for about 16 h. Subsequently, water was added and extraction was effected with ethyl acetate. The organic extract was washed successively with water and saturated sodium chloride solution, dried over anhydrous magnesium sulfate and concentrated. The residue was purified by means of MPLC (Biotage Isolera One, SNAP KP-Sil cartridge, 100 g of silica gel, eluent: cyclohexane/ethyl acetate 2:1). After concentration of the product fractions and drying under high vacuum, 1.17 g (85% of theory) of the title compound were obtained.

¹H-NMR (400 MHz, DMSO-d₆, δ/ppm): 10.09 (s, 1H), 4.32-3.79 (m, 2H), 2.78 (s, 3H), 2.30-2.13 (m, 1H), 1.78-1.63 (m, 1H), 1.54-1.44 (m, 1H), 1.35 (s, 3H), 1.02-0.75 (m, 4H).

LC/MS (Method 1, ESIpos): Rₜ=1.82 min, m/z=355.09 [M+H]⁺.

### Example 67A

### [6-Formyl-5-methyl-3-(1-methylcyclopropyl)-2,4-dioxo-3,4-dihydrothieno[2,3-d]pyrimidin-1(2H)-yl]acetonitrile

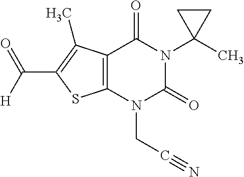 [SMILES: Cc1c(C=O)sc2c1c(=O)n(C1(C)CC1)c(=O)n2CC#N]

Analogously to the method described in Ex. 63A, 500 mg (1.89 mmol) of the compound from Ex. 32A and 454 mg (3.78 mmol) of bromoacetonitrile were used to prepare 455 mg (63% of theory, 80% purity) of the title compound.

¹H-NMR (400 MHz, DMSO-d₆, δ/ppm): 10.10 (s, 1H), 5.18 (br. d, 2H), 2.79 (s, 3H), 1.36 (s, 3H), 0.96-0.84 (m, 4H).

LC/MS (Method 2, ESIpos): Rₜ=0.78 min, m/z=304 [M+H]⁺.

### Example 68A

### 1-(2-Methoxyethyl)-5-methyl-3-(1-methylcyclopropyl)-2,4-dioxo-1,2,3,4-tetrahydrothieno[2,3-d]pyrimidine-6-carbaldehyde

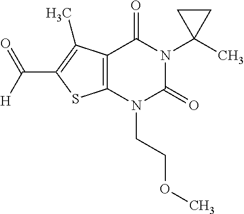 [SMILES: COCCn1c(=O)n(C2(C)CC2)c(=O)c2c(C)c(C=O)sc21]

### Process A:

2.36 g (8.94 mmol) of the compound from Ex. 32A were dissolved in 47 ml of anhydrous DMF, and 3.09 g (22.4 mmol) of potassium carbonate were added. The mixture was stirred at RT for 15 min. Then 2.5 ml (26.8 mmol) of 2-bromoethyl methyl ether were added. The reaction mixture was stirred at 50° C. for about 16 h. After cooling to RT, water was added, whereupon a portion of the product precipitated out, which was filtered off with suction, washed with a little water and dried under high vacuum. This gave a first fraction of 1.60 g (49% of theory, 89% purity) of the title compound. The filtrate and the wash water were combined and extracted with dichloromethane. The organic extract was washed with saturated sodium chloride solution and concentrated to dryness. In this way, a further 940 mg (23% of theory, 71% purity) of the product were isolated. A total of 2.54 g (73% of theory, 83% purity) of the title compound was thus obtained, which was used for subsequent reactions without further purification.

### Process B:

To a solution of 33.5 g (114 mmol) of the compound from Ex. 45A in 88 ml (1.14 mol) of DMF were cautiously added 127 ml (1.37 mol) of phosphorus oxychloride. After the strongly exothermic reaction had abated, the mixture was stirred for another 15 min. Then it was stirred cautiously into 3.5 litres of water. After stirring at RT for about 15 h, the precipitated solids were filtered off with suction, washed to neutrality with water and dried under high vacuum. The product was isolated therefrom by means of MPLC (Biotage Isolera One, SNAP KP-Sil cartridge, 340 g of silica gel, eluent: cyclohexane/ethyl acetate 2:1). After concentration, this gave a first fraction of the title compound (13.2 g) in pure form and a second, contaminated fraction. The contaminated fraction was stirred with ethyl acetate at RT for 16 h. The solids were filtered off with suction and, after drying, gave a second fraction of the title compound (11.0 g) in pure form. A total of 24.2 g (66% of theory) of the title compound was thus obtained.

¹H-NMR (400 MHz, DMSO-d₆, δ/ppm): 10.07 (s, 1H), 4.25-3.88 (m, 2H), 3.64 (br. t, 2H), 3.24 (s, 3H), 2.77 (s, 3H), 1.35 (s, 3H), 1.02-0.74 (m, 4H).

LC/MS (Method 2, ESIpos): Rₜ=0.82 min, m/z=323 [M+H]⁺.

### Example 69A

### 5-Methyl-3-(1-methylcyclopropyl)-2,4-dioxo-1-[2-(trifluoromethoxy)ethyl]-1,2,3,4-tetrahydrothieno[2,3-d]pyrimidine-6-carbaldehyde

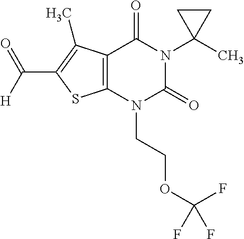 [SMILES: Cc1c(C=O)sc2c1c(=O)n(C1(C)CC1)c(=O)n2CCOC(F)(F)F]

500 mg (1.89 mmol) of the compound from Ex. 32A and 654 mg (4.73 mmol) of potassium carbonate were stirred in 10 ml of anhydrous DMF at RT for 15 min, before 730 mg (3.78 mmol) of 1-bromo-2-(trifluoromethoxy)ethane [commercially available; lit.: P. E. Aldrich, W. A. Sheppard, J. Org. Chem. 29 (1), 11-15 (1964)] were added. Then the reaction mixture was stirred first at RT for 2.5 days and then at 50° C. for about another 5 h. Then water was added and extraction was effected with ethyl acetate. The organic extract was washed successively with water and saturated sodium chloride solution, dried over anhydrous magnesium sulfate and concentrated. Drying under high vacuum gave 668 mg (93% of theory) of the title compound.

¹H-NMR (400 MHz, DMSO-d₆, δ/ppm): 10.08 (s, 1H), 4.43-4.38 (m, 2H), 4.36-4.10 (m, 2H), 2.78 (s, 3H), 1.35 (s, 3H), 0.99-0.78 (m, 4H).

LC/MS (Method 1, ESIpos): Rₜ=1.88 min, m/z=377.08 [M+H]⁺.

### Example 70A

### 1-(2-Ethoxyethyl)-5-methyl-3-(1-methylcyclopropyl)-2,4-dioxo-1,2,3,4-tetrahydrothieno[2,3-d]pyrimidine-6-carbaldehyde

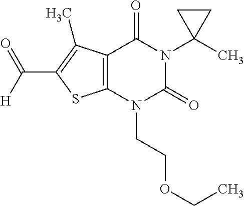 [SMILES: CCOCCn1c(=O)n(C2(C)CC2)c(=O)c2c(C)c(C=O)sc21]

Analogously to the method described in Ex. 65A, 500 mg (1.89 mmol) of the compound from Ex. 32A and 579 mg (3.78 mmol) of 2-bromoethyl ethyl ether were used to prepare 448 mg (70% of theory) of the title compound.

¹H-NMR (400 MHz, DMSO-d₆, δ/ppm): 10.07 (s, 1H), 4.22-3.89 (m, 2H), 3.67 (t, 2H), 3.44 (q, 2H), 2.77 (s, 3H), 1.35 (s, 3H), 0.98-0.77 (m, 4H).

LC/MS (Method 1, ESIpos): Rₜ=1.73 min, m/z=337.12 [M+H]⁺.

### Example 71A

### 1-(2-Isopropoxyethyl)-5-methyl-3-(1-methylcyclopropyl)-2,4-dioxo-1,2,3,4-tetrahydrothieno[2,3-d]pyrimidine-6-carbaldehyde

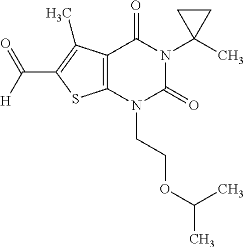 [SMILES: Cc1c(C=O)sc2c1c(=O)n(C1(C)CC1)c(=O)n2CCOC(C)C]

500 mg (1.89 mmol) of the compound from Ex. 32A and 523 mg (3.78 mmol) of potassium carbonate were stirred in 10 ml of anhydrous DMF at RT for 15 min, before 474 mg (2.84 mmol) of 2-bromoethyl isopropyl ether were added. Then the reaction mixture was stirred first at RT for 2.5 days and then at 50° C. for 8 h. Since the conversion was still incomplete, a further 131 mg (0.946 mmol) of potassium carbonate and 158 mg (0.946 mmol) of 2-bromoethyl isopropyl ether were added and the stirring was continued at 50° C. for about 16 h. Subsequently, the reaction mixture, after being cooled down to RT, was admixed with water and stirred at RT for 30 min. The product which precipitated out was filtered off with suction, washed with water and dried under high vacuum. 485 mg (73% of theory) of the title compound were obtained.

¹H-NMR (400 MHz, DMSO-d₆, δ/ppm): 10.07 (s, 1H), 4.20-3.86 (m, 2H), 3.66 (t, 2H), 3.56 (sept, 1H), 2.77 (s, 3H), 1.35 (s, 3H), 1.00 (d, 6H), 0.96-0.78 (m, 4H).

LC/MS (Method 1, ESIpos): Rₜ=1.87 min, m/z=351.14 [M+H]⁺.

### Example 72A

### 5-Methyl-3-(1-methylcyclopropyl)-2,4-dioxo-1-[(2R)-tetrahydrofuran-2-ylmethyl]-1,2,3,4-tetrahydrothieno[2,3-d]pyrimidine-6-carbaldehyde

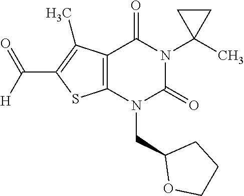 [SMILES: Cc1c(C=O)sc2c1c(=O)n(C1(C)CC1)c(=O)n2C[C@H]1CCCO1]

500 mg (1.89 mmol) of the compound from Ex. 32A and 654 mg (4.73 mmol) of potassium carbonate were stirred in 10 ml of anhydrous DMF at RT for 15 min, before 624 mg (3.78 mmol) of (2R)-2-(bromomethyl)tetrahydrofuran were added. Then the reaction mixture was stirred first at RT for 2.5 days. Since the conversion was only low, the mixture was then stirred at 50° C. for about 20 h. Since the conversion was still low, the reaction mixture was transferred to a microwave oven (Biotage Initiator with dynamic control of irradiation power) and heated therein to 100° C. for 9 h.

After cooling to RT, water was added to the reaction mixture, which was extracted with ethyl acetate. The organic extract was washed successively with water and saturated sodium chloride solution, dried over anhydrous magnesium sulfate and concentrated. The residue was purified by means of MPLC (Biotage Isolera One, SNAP KP-Sil cartridge, 100 g of silica gel, eluent: cyclohexane/ethyl acetate 2:1). After concentration of the product fractions and drying under high vacuum, 397 mg (57% of theory, 96% purity) of the title compound were obtained.

¹H-NMR (400 MHz, DMSO-d₆, δ/ppm): 10.07 (s, 1H), 4.30-3.94 (m, 2H), 3.88-3.54 (m, 3H), 2.77 (s, 3H), 2.06-1.74 (m, 3H), 1.73-1.59 (m, 1H), 1.35 (s, 3H), 0.98-0.75 (m, 4H).

LC/MS (Method 1, ESIpos): Rₜ=1.70 min, m/z=349.12 [M+H]⁺.

### Example 73A

### 5-Methyl-3-(1-methylcyclopropyl)-2,4-dioxo-1-[(2S)-tetrahydrofuran-2-ylmethyl]-1,2,3,4-tetrahydrothieno[2,3-d]pyrimidine-6-carbaldehyde

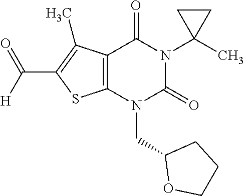 [SMILES: Cc1c(C=O)sc2c1c(=O)n(C1(C)CC1)c(=O)n2C[C@@H]1CCCO1]

Analogously to the method described in Ex. 65A, 500 mg (1.89 mmol) of the compound from Ex. 32A and 624 mg (3.78 mmol) of (2S)-2-(bromomethyl)tetrahydrofuran were used to prepare 527 mg (75% of theory, 95% purity) of the title compound.

¹H-NMR (400 MHz, DMSO-d₆, δ/ppm): 10.07 (s, 1H), 4.30-3.96 (m, 2H), 3.89-3.56 (m, 3H), 2.77 (s, 3H), 2.06-1.75 (m, 3H), 1.72-1.60 (m, 1H), 1.35 (s, 3H), 1.00-0.77 (m, 4H).

LC/MS (Method 1, ESIpos): Rₜ=1.70 min, m/z=349.12 [M+H]⁺.

### Example 74A

### 1-(2-Methoxyethyl)-5-methyl-2,4-dioxo-3-[1-(trifluoromethyl)cyclopropyl]-1,2,3,4-tetrahydrothieno[2,3-d]pyrimidine-6-carbaldehyde

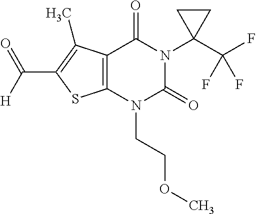 [SMILES: COCCn1c(=O)n(C2(C(F)(F)F)CC2)c(=O)c2c(C)c(C=O)sc21]

1.80 g (5.66 mmol) of the compound from Ex. 33A and 1.95 g (14.1 mmol) of potassium carbonate were stirred in 30 ml of anhydrous DMF at RT for 15 min, before 1 ml (11.3 mmol) of 2-bromoethyl methyl ether was added. Then the reaction mixture was stirred at 50° C. for about 16 h. After this time, a further 782 mg (5.66 mmol) of potassium carbonate and 531 μl (5.66 mmol) of 2-bromoethyl methyl ether were added and the stirring was continued at 50° C. for 7 h. After cooling to RT, water was added and extraction was effected with ethyl acetate. The organic extract was washed successively with water and saturated sodium chloride solution, dried over anhydrous magnesium sulfate, filtered and concentrated. The product was isolated by means of MPLC (Biotage Isolera One, SNAP KP-Sil cartridge, 100 g of silica gel, eluent: cyclohexane/ethyl acetate 2:1). After concentration of the product fractions and drying under high vacuum, 1.50 g (70% of theory) of the title compound were obtained.

¹H-NMR (400 MHz, DMSO-d₆, δ/ppm): 10.09 (s, 1H), 4.19-3.99 (m, 2H), 3.64 (t, 2H), 3.25 (s, 3H), 2.77 (s, 3H), 1.68-1.52 (m, 2H), 1.45-1.30 (m, 2H).

LC/MS (Method 1, ESIpos): Rₜ=1.71 min, m/z=377.08 [M+H]⁺.

### Example 75A

### 1-(3-Fluoropropyl)-5-methyl-3-(2-methylcyclopropyl)-2,4-dioxo-1,2,3,4-tetrahydrothieno[2,3-d]pyrimidine-6-carbaldehyde (trans racemate)

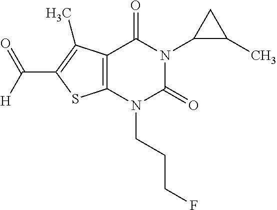 [SMILES: Cc1c(C=O)sc2c1c(=O)n(C1CC1C)c(=O)n2CCCF]

To a solution of 479 mg (1.68 mmol) of the compound from Ex. 34A in 20 ml of DMF were added 582 mg (4.21 mmol) of potassium carbonate, and the mixture was stirred at RT for 15 min. Then 951 mg (5.05 mmol) of 1-fluoro-3-iodopropane were added, and the mixture was stirred at 50° C. for 20 h. The DMF was very substantially distilled off and the residue obtained was partitioned between semisaturated sodium chloride solution (100 ml) and ethyl acetate (50 ml). The water phase was extracted with ethyl acetate. The combined organic phases were dried over sodium sulfate, filtered and concentrated. The residue obtained was chromatographed using a silica gel cartridge (Biotage, 100 g of silica gel, eluent: hexane/ethyl acetate 92:8-34:66). 462 mg (82% of theory) of the title compound were obtained.

¹H-NMR (400 MHz, DMSO-d₆, δ/ppm): 10.08 (s, 1H), 4.60 (t, 1H), 4.48 (t, 1H), 4.07-3.94 (m, 2H), 2.77 (s, 3H), 2.28-2.22 (m, 1H), 2.14-2.06 (m, 1H), 2.03 (t, 1H), 1.15 (d, 3H), 1.07-0.96 (m, 1H), 0.89-0.80 (m, 2H).

LC/MS (Method 4, ESIpos): Rₜ=1.09 min, m/z=325 [M+H]⁺.

### Example 76A

### 5-Methyl-3-(2-methylcyclopropyl)-2,4-dioxo-1-(3,3,3-trifluoropropyl)-1,2,3,4-tetrahydrothieno[2,3-d]pyrimidine-6-carbaldehyde (trans racemate)

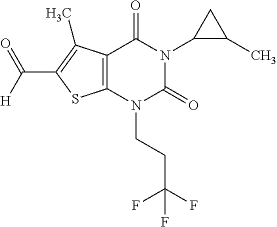 [SMILES: Cc1c(C=O)sc2c1c(=O)n(C1CC1C)c(=O)n2CCC(F)(F)F]

To a solution of 479 mg (1.68 mmol) of the compound from Ex. 34A in 20 ml of DMF were added 582 mg (4.21 mmol) of potassium carbonate, and the mixture was stirred at RT for 15 min. Then 1.17 g (5.05 mmol) of 1,1,1-trifluoro-3-iodopropane were added, and the mixture was stirred at 50° C. for 20 h. The DMF was very substantially distilled off and the residue obtained was partitioned between semisaturated sodium chloride solution (100 ml) and ethyl acetate (50 ml). The water phase was extracted with ethyl acetate. The combined organic phases were dried over sodium sulfate, filtered and concentrated. The residue obtained was chromatographed using a silica gel cartridge (Biotage, 100 g of silica gel, eluent: hexane/ethyl acetate 94:6→50:50). In this way, 517 mg (85% of theory) of the title compound were obtained.

¹H-NMR (400 MHz, DMSO-d₆, δ/ppm): 10.09 (s, 1H), 4.13 (d, 2H), 2.84-2.69 (m, 5H), 2.30-2.23 (m, 1H), 1.15 (d, 3H), 1.07-0.95 (m, 1H), 0.90-0.81 (m, 2H).

LC/MS (Method 4, ESIpos): Rₜ=1.2 min, m/z=361 [M+H]⁺.

### Example 77A

### 1-(2-Methoxyethyl)-5-methyl-3-(2-methylcyclopropyl)-2,4-dioxo-1,2,3,4-tetrahydrothieno[2,3-d]pyrimidine-6-carbaldehyde (trans racemate)

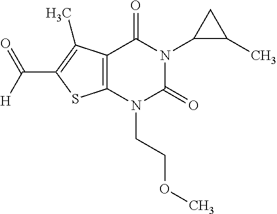 [SMILES: COCCn1c(=O)n(C2CC2C)c(=O)c2c(C)c(C=O)sc21]

To a solution of 479 mg (1.68 mmol) of the compound from Ex. 34A in 17 ml of DMF were added 582 mg (4.21 mmol) of potassium carbonate, and the mixture was stirred at RT for 15 min. 703 mg (5.05 mmol) of 2-bromoethyl methyl ether were then added, and the mixture was stirred at 50° C. for 70 h. The DMF was very substantially distilled off and the residue obtained was partitioned between semisaturated sodium chloride solution (100 ml) and ethyl acetate (50 ml). The water phase was extracted with ethyl acetate. The combined organic phases were dried over sodium sulfate, filtered and concentrated. The residue obtained was chromatographed using a silica gel cartridge (Biotage, 100 g of silica gel, eluent: hexane/ethyl acetate 92:8→34:66). In this way, 317 mg (58% of theory) of the title compound were obtained. In addition, 136 mg (21% of theory, 86% purity) of a second fraction of the title compound were isolated.

¹H-NMR (400 MHz, DMSO-d₆, δ/ppm): 10.07 (s, 1H), 4.11-3.99 (m, 2H), 3.63 (t, 2H), 3.23 (s, 3H), 2.76 (s, 3H), 2.26 (dt, 1H), 1.15 (d, 3H), 1.07-0.96 (m, 1H), 0.90-0.80 (m, 2H).

LC/MS (Method 4, ESIpos): Rₜ=1.05 min, m/z=323 [M+H]⁺.

### Example 78A

### 3-(2,2-Dimethylcyclopropyl)-1-(3-fluoropropyl)-5-methyl-2,4-dioxo-1,2,3,4-tetrahydrothieno[2,3-d]pyrimidine-6-carbaldehyde (racemate)

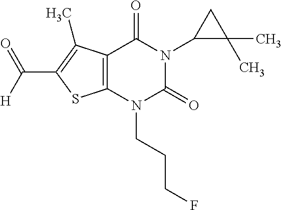 [SMILES: Cc1c(C=O)sc2c1c(=O)n(C1CC1(C)C)c(=O)n2CCCF]

Analogously to Ex. 75A, 565 mg (1.68 mmol) of the compound from Ex. 35A in 24 ml of DMF were reacted with 701 mg (5.07 mmol) of potassium carbonate and 1.14 g (6.09 mmol) of 1-fluoro-3-iodopropane. 609 mg (87% of theory) of the title compound were obtained.

¹H-NMR (400 MHz, DMSO-d₆, δ/ppm): 10.08 (s, 1H), 4.60 (t, 1H), 4.48 (t, 1H), 4.03 (br. d, 2H), 2.77 (s, 3H), 2.40 (dd, 1H), 2.15-1.99 (m, 2H), 1.17 (s, 3H), 1.05 (dd, 1H), 0.88 (s, 3H), 0.72 (dd, 1H).

LC/MS (Method 4, ESIpos): Rₜ=1.22 min, m/z=339 [M+H]⁺.

### Example 79A

### 3-(2,2-Dimethylcyclopropyl)-5-methyl-2,4-dioxo-1-(3,3,3-trifluoropropyl)-1,2,3,4-tetrahydrothieno[2,3-d]pyrimidino-6-carbaldehyde (racemate)

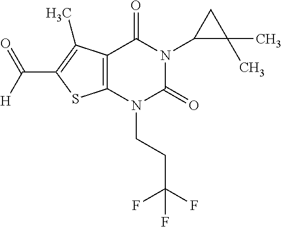 [SMILES: Cc1c(C=O)sc2c1c(=O)n(C1CC1(C)C)c(=O)n2CCC(F)(F)F]

To a solution of 500 mg (1.79 mmol) of the compound from Ex. 35A in 22 ml of DMF were added 620 mg (4.49 mmol) of potassium carbonate, and the mixture was stirred at RT for 15 min. Then 1.24 g (5.39 mmol) of 1,1,1-trifluoro-3-iodopropane were added, and the mixture was stirred at 50° C. for 15 h. The DMF was very substantially distilled off and the residue obtained was partitioned between semisaturated sodium chloride solution (100 ml) and ethyl acetate (50 ml). The water phase was extracted with ethyl acetate. The combined organic phases were dried over sodium sulfate, filtered and concentrated. The residue obtained was chromatographed using a silica gel cartridge (Biotage, 100 g of silica gel, eluent: hexane/ethyl acetate 94:6→50:50). In this way, 600 mg (88% of theory) of the title compound were obtained.

¹H-NMR (400 MHz, DMSO-d₆, δ/ppm): 10.09 (s, 1H), 4.15 (br. d, 2H), 2.85-2.71 (m, 5H), 2.42 (dd, 1H), 1.17 (s, 3H), 1.06 (dd, 1H), 0.87 (s, 3H), 0.73 (dd, 1H).

LC/MS (Method 4, ESIpos): Rₜ=1.32 min, m/z=375 [M+H]⁺.

### Example 80A

### 3-(2,2-Dimethylcyclopropyl)-1-(2-methoxyethyl)-5-methyl-2,4-dioxo-1,2,3,4-tetrahydrothieno[2,3-d]pyrimidine-6-carbaldehyde (racemate)

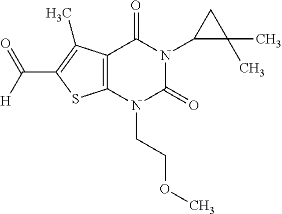 [SMILES: COCCn1c(=O)n(C2CC2(C)C)c(=O)c2c(C)c(C=O)sc21]

Analogously to Ex. 77A, 500 mg (1.79 mmol) of the compound from Ex. 35A in 18 ml of DMF were reacted with 621 mg (4.49 mmol) of potassium carbonate and 749 g (5.39 mmol) of 2-bromoethyl methyl ether. 485 mg (77% of theory) of the title compound were obtained.

¹H-NMR (400 MHz, DMSO-d₆, δ/ppm): 10.07 (s, 1H), 4.19-4.09 (m, 1H), 4.06-3.95 (m, 1H), 3.69-3.57 (m, 2H), 3.22 (s, 3H), 2.76 (s, 3H), 2.42 (dd, 1H), 1.17 (s, 3H), 1.06 (dd, 1H), 0.87 (s, 3H), 0.73 (dd, 1H).

LC/MS (Method 4, ESIpos): Rₜ=1.17 min, m/z=337 [M+H]⁺.

### Example 81A

### 3-(1-Ethylcyclopropyl)-5-methyl-2,4-dioxo-1-(3,3,3-trifluoropropyl)-1,2,3,4-tetrahydrothieno[2,3-d]pyrimidine-6-carbaldehyde

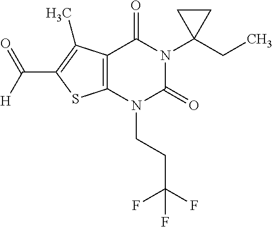 [SMILES: CCC1(n2c(=O)c3c(C)c(C=O)sc3n(CCC(F)(F)F)c2=O)CC1]

Analogously to the method described in Ex. 52A, 430 mg (1.54 mmol) of the compound from Ex. 36A and 692 mg (3.09 mmol) of 1,1,1-trifluoro-3-iodopropane were used to prepare 345 mg (59% of theory) of the title compound. The reaction was effected here not at 50° C. but at 60° C., and the reaction time was 26 h.

¹H-NMR (400 MHz, DMSO-d₆, δ/ppm): 10.09 (s, 1H), 4.23-4.04 (m, 2H), 2.86-2.70 (m, 2H), 2.78 (s, 3H), 1.70 (q, 2H), 1.04-0.84 (m, 4H), 0.83 (t, 3H).

LC/MS (Method 1, ESIpos): Rₜ=2.02 min, m/z=375.10 [M+H]⁺.

### Example 82A

### 3-(1-Ethylcyclopropyl)-1-(2-methoxyethyl)-5-methyl-2,4-dioxo-1,2,3,4-tetrahydrothieno[2,3-d]pyrimidine-6-carbaldehyde

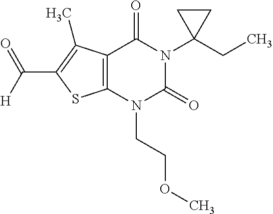 [SMILES: CCC1(n2c(=O)c3c(C)c(C=O)sc3n(CCOC)c2=O)CC1]

Analogously to the method described in Ex. 52A, 430 mg (1.54 mmol) of the compound from Ex. 36A and 644 mg (4.64 mmol) of 2-bromoethyl methyl ether were used to prepare 428 mg (80% of theory) of the title compound. The reaction in this case was not effected at 50° C., but at 60° C.

¹H-NMR (400 MHz, DMSO-d₆, δ/ppm): 10.07 (s, 1H), 4.17-3.94 (m, 2H), 3.63 (t, 2H), 3.24 (s, 3H), 2.76 (s, 3H), 1.70 (q, 2H), 1.03-0.77 (m, 4H), 0.83 (t, 3H).

LC/MS (Method 1, ESIpos): Rₜ=1.77 min, m/z=337.12 [M+H]⁺.

### Example 83A

### 3-Cyclobutyl-5-methyl-2,4-dioxo-1-(3,3,3-trifluoropropyl)-1,2,3,4-tetrahydrothieno[2,3-d]pyrimidine-6-carbaldehyde

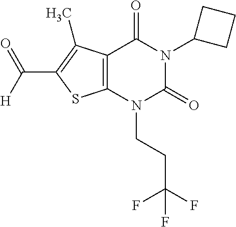 [SMILES: Cc1c(C=O)sc2c1c(=O)n(C1CCC1)c(=O)n2CCC(F)(F)F]

2.50 g (9.46 mmol) of the compound from Ex. 37A and 3.27 g (23.6 mmol) of potassium carbonate were stirred in 50 ml of anhydrous DMF at RT for 15 min, before 3.3 ml (28.4 mmol) of 1,1,1-trifluoro-3-iodopropane were added. Then the reaction mixture was stirred first at RT for about 16 h and then at 50° C. for 4 h. After cooling to RT, water was added thereto and it was stirred at RT for 30 min. The product which precipitated out was filtered off with suction, washed with water and dried under high vacuum. 2.90 g (85% of theory) of the title compound were obtained.

¹H-NMR (400 MHz, DMSO-d₆, δ/ppm): 10.10 (s, 1H), 5.17 (quin, 1H), 4.14 (t, 2H), 2.88-2.70 (m, 4H), 2.78 (s, 3H), 2.27-2.11 (m, 2H), 1.90-1.64 (m, 2H).

LC/MS (Method 1, ESIpos): Rₜ=2.07 min, m/z=361.08 [M+H]⁺.

### Example 84A

### 3-Cyclobutyl-1-(2-methoxyethyl)-5-methyl-2,4-dioxo-1,2,3,4-tetrahydrothieno[2,3-d]pyrimidine-6-carbaldehyde

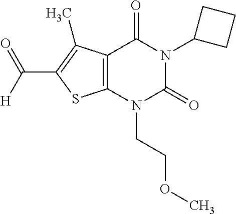 [SMILES: COCCn1c(=O)n(C2CCC2)c(=O)c2c(C)c(C=O)sc21]

2.50 g (9.46 mmol) of the compound from Ex. 37A and 3.27 g (23.6 mmol) of potassium carbonate were stirred in 50 ml of anhydrous DMF at RT for 15 min, before 1.8 ml (28.4 mmol) of 2-bromoethyl methyl ether were added. Then the reaction mixture was stirred first at RT for about 16 h and then at 50° C. for 24 h. After cooling to RT, water was added thereto and it was stirred at RT for 30 min. The precipitated solid was filtered off with suction, washed with water and dried under high vacuum. The product was isolated therefrom by means of MPLC (Biotage Isolera One, SNAP KP-Sil cartridge, 100 g of silica gel, eluent: cyclohexane/ethyl acetate 2:1). After concentration of the product fractions and drying under high vacuum, 2.25 g (73% of theory) of the title compound were obtained.

¹H-NMR (400 MHz, DMSO-d₆, δ/ppm): 10.08 (s, 1H), 5.17 (quin, 1H), 4.07 (t, 2H), 3.65 (t, 2H), 3.25 (s, 3H), 2.88-2.74 (m, 2H), 2.76 (s, 3H), 2.26-2.13 (m, 2H), 1.89-1.65 (m, 2H).

LC/MS (Method 2, ESIpos): Rₜ=0.96 min, m/z=323 [M+H]⁺.

### Example 85A

### 3-(3,3-Difluorocyclobutyl)-5-methyl-2,4-dioxo-1-(3,3,3-trifluoropropyl)-1,2,3,4-tetrahydrothieno[2,3-d]pyrimidine-6-carbaldehyde

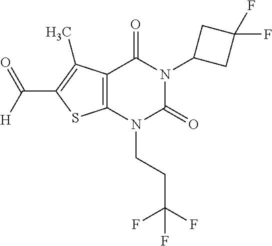 [SMILES: Cc1c(C=O)sc2c1c(=O)n(C1CC(F)(F)C1)c(=O)n2CCC(F)(F)F]

To a solution of 2.0 g (6.60 mmol) of the compound from Ex. 38A in 35 ml of DMF were added 2.30 g (16.6 mmol) of potassium carbonate, and the mixture was stirred at RT for 15 min. Then 2.3 ml (20.0 mmol) of 1,1,1-trifluoro-3-iodopropane were added. Since the conversion was still incomplete after stirring at RT for 3 days, the mixture was first stirred at 50° C. for 4.5 h, and then a further 0.92 g (6.60 mmol) of potassium carbonate and 0.78 ml (6.0 mmol) of 1,1,1-trifluoro-3-iodopropane were added. The reaction mixture was then stirred at 60° C. overnight. After cooling to RT, the mixture was diluted with water and stirred for 30 min. The precipitated product was filtered off with suction, washed to neutrality with water and dried. 2.15 g (78% of theory) of the title compound were obtained, which was used for subsequent reactions without further purification.

LC/MS (Method 2, ESIpos): Rₜ=1.08 min, m/z=397 [M+H]⁺.

### Example 86A

### 3-(3,3-Difluorocyclobutyl)-1-(2-methoxyethyl)-5-methyl-2,4-dioxo-1,2,3,4-tetrahydrothieno[2,3-d]pyrimidine-6-carbaldehyde

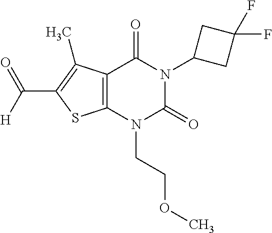 [SMILES: COCCn1c(=O)n(C2CC(F)(F)C2)c(=O)c2c(C)c(C=O)sc21]

To a solution of 2.0 g (6.60 mmol) of the compound from Ex. 38A in 35 ml of DMF were added 2.30 g (16.6 mmol) of potassium carbonate, and the mixture was stirred at RT for 15 min. Then 1.9 ml (20.0 mmol) of 2-bromoethyl methyl ether were added. Since the conversion was still incomplete after stirring at RT for 3 days, the mixture was first stirred at 50° C. for 4.5 h, then a further 0.92 g (6.60 mmol) of potassium carbonate and 0.78 ml (6.0 mmol) of 2-bromoethyl methyl ether were added and the stirring was then continued at 60° C. overnight. After cooling to RT, the mixture was diluted with water and stirred for 30 min. The precipitated product was filtered off with suction, washed to neutrality with water and dried. 2.15 g (78% of theory, 87% purity) of the title compound were obtained, which was used for subsequent reactions without further purification.

LC/MS (Method 2, ESIpos): Rₜ=0.96 min, m/z=359 [M+H]⁺.

### Example 87A

### 5-Methyl-3-(oxetan-3-yl)-2,4-dioxo-1-(3,3,3-trifluoropropyl)-1,2,3,4-tetrahydrothieno[2,3-d]pyrimidine-6-carbaldehyde

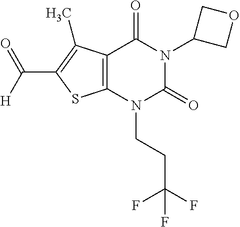 [SMILES: Cc1c(C=O)sc2c1c(=O)n(C1COC1)c(=O)n2CCC(F)(F)F]

To a solution of 2.16 g (5.23 mmol) of the compound from Ex. 47A in 50 ml of anhydrous THF were added dropwise, at −78° C., 6.3 ml (10.7 mmol) of a 1.7 M solution of tert-butyllithium in pentane. After stirring—still at −78° C.—for 1 h, a solution of 2 ml (26.1 mmol) of DMF in 10 ml of THF was added. After a further hour at −78° C., saturated aqueous ammonium chloride solution was added and then the mixture was warmed to RT. It was extracted with diethyl ether. The organic extract was dried over anhydrous magnesium sulfate, filtered and concentrated. The product was isolated by means of MPLC (Biotage Isolera One, SNAP KP-Sil cartridge, 100 g of silica gel, eluent: cyclohexane/ethyl acetate 2:1). After concentration of the product fractions and drying under high vacuum, 785 mg (41% of theory) of the title compound were obtained.

¹H-NMR (400 MHz, DMSO-d₆, δ/ppm): 10.10 (s, 1H), 5.05 (quin, 1H), 4.78-4.66 (m, 4H), 4.14 (t, 2H), 2.85-2.70 (m, 2H), 2.76 (s, 3H).

LC/MS (Method 1, ESIpos): Rₜ=1.54 min, m/z=363.06 [M+H]⁺.

### Example 88A

### 5-Methyl-3-(1-methylcyclobutyl)-2,4-dioxo-1-(3,3,3-trifluoropropyl)-1,2,3,4-tetrahydrothieno[2,3-d]pyrimidine-6-carbaldehyde

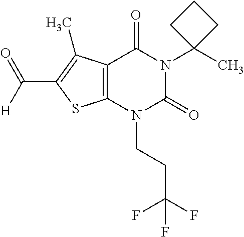 [SMILES: Cc1c(C=O)sc2c1c(=O)n(C1(C)CCC1)c(=O)n2CCC(F)(F)F]

2.95 g (10.6 mmol) of the compound from Ex. 39A and 3.66 g (26.5 mmol) of potassium carbonate were stirred in 60 ml of anhydrous DMF at RT for 15 min, before 3.7 ml (31.8 mmol) of 1,1,1-trifluoro-3-iodopropane were added. Then the reaction mixture was stirred at 50° C. for about 16 h. After cooling to RT, water was added and the mixture was extracted with ethyl acetate. The organic extract was washed with saturated sodium chloride solution, dried over anhydrous magnesium sulfate, filtered and concentrated. The product was purified by stirring with pentane/dichloromethane (25:1) at RT. After the solids had been filtered off with suction and dried under high vacuum, 3.25 g (77% of theory; 95% purity) of the title compound were obtained.

¹H-NMR (400 MHz, DMSO-d₆, δ/ppm): 10.09 (s, 1H), 4.12 (t, 2H), 2.85-2.69 (m, 2H), 2.76 (s, 3H), 2.40-2.21 (m, 4H), 1.84-1.57 (m, 2H), 1.53 (s, 3H).

LC/MS (Method 1, ESIpos): Rₜ=2.11 min, m/z=375.10 [M+H]⁺.

### Example 89A

### 1-(2-Methoxyethyl)-5-methyl-3-(1-methylcyclobutyl)-2,4-dioxo-1,2,3,4-tetrahydrothieno[2,3-d]pyrimidine-6-carbaldehyde

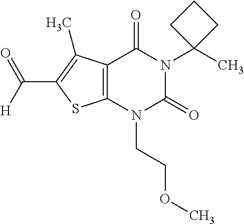 [SMILES: COCCn1c(=O)n(C2(C)CCC2)c(=O)c2c(C)c(C=O)sc21]

3.0 g (10.8 mmol) of the compound from Ex. 39A and 3.72 g (26.9 mmol) of potassium carbonate were stirred in 60 ml of anhydrous DMF at RT for 15 min, before 2 ml (21.6 mmol) of 2-bromoethyl methyl ether were added. Then the reaction mixture was stirred at 50° C. for about 16 h. After cooling to RT, water was added thereto and it was stirred at RT for 30 min. The precipitated solids were filtered off with suction, washed with a little water and dried under high vacuum. This gave a first fraction of 2.27 g of the title compound. The filtrate contaminated with the wash water was extracted with ethyl acetate. The organic extract was washed with saturated sodium chloride solution, dried over anhydrous magnesium sulfate, filtered and concentrated. A second fraction of 0.73 g of the title compound was isolated from the residue by means of MPLC (Biotage Isolera One, SNAP KP-Sil cartridge, 100 g of silica gel, eluent: cyclohexane/ethyl acetate 2:1). A total of 3.0 g (82% of theory) of the title compound was thus obtained.

¹H-NMR (400 MHz, DMSO-d₆, δ/ppm): 10.07 (s, 1H), 4.04 (t, 2H), 3.63 (t, 2H), 3.25 (s, 3H), 2.74 (s, 3H), 2.40-2.20 (m, 4H), 1.82-1.58 (m, 2H), 1.53 (s, 3H).

LC/MS (Method 1, ESIpos): Rₜ=1.88 min, m/z=337.12 [M+H]⁺.

### Example 90A

### 3-(trans-3-Methoxycyclobutyl)-5-methyl-2,4-dioxo-1-(3,3,3-trifluoropropyl)-1,2,3,4-tetrahydrothieno[2,3-d]pyrimidine-6-carbaldehyde

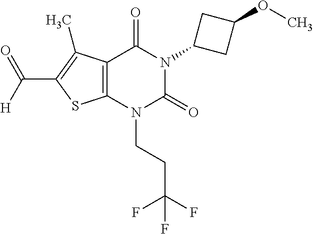 [SMILES: CO[C@H]1C[C@H](n2c(=O)c3c(C)c(C=O)sc3n(CCC(F)(F)F)c2=O)C1]

To a solution of 2.23 g (7.09 mmol, 94% purity) of the compound from Ex. 40A in 37 ml of DMF were added 2.45 g (17.7 mmol) of potassium carbonate, and the mixture was stirred at RT for 15 min. Then 2.5 ml (21.3 mmol) of 1,1,1-trifluoro-3-iodopropane were added. Since the conversion was still incomplete after stirring at RT for 16 h, the mixture was stirred at 50° C. for a further 2 days. After cooling to RT, the mixture was diluted with water and stirred for 30 min. The precipitated product was filtered off with suction, washed to neutrality with water and dried. 2.26 g (74% of theory, 91% purity) of the title compound were obtained, which was used for subsequent reactions without further purification.

¹H-NMR (400 MHz, DMSO-d₆, δ/ppm): 10.10 (s, 1H), 5.48-5.39 (m, 1H), 4.18-4.10 (m, 3H), 3.17 (s, 3H), 2.96-2.87 (m, 2H), 2.85-2.71 (m, 5H), 2.29-2.22 (m, 2H).

LC/MS (Method 2, ESIpos): Rₜ=1.01 min, m/z=391 [M+H]⁺.

### Example 91A

### 3-(trans-3-Methoxycyclobutyl)-1-(2-methoxyethyl)-5-methyl-2,4-dioxo-1,2,3,4-tetrahydrothieno[2,3-d]pyrimidine-6-carbaldehyde

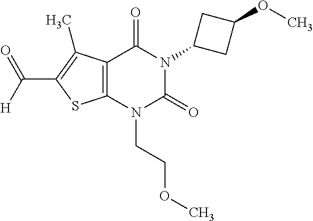 [SMILES: COCCn1c(=O)n([C@H]2C[C@H](OC)C2)c(=O)c2c(C)c(C=O)sc21]

To a solution of 2.23 g (7.09 mmol, 94% purity) of the compound from Ex. 40A in 37 ml of DMF were added 2.45 g (17.7 mmol) of potassium carbonate, and the mixture was stirred at RT for 15 min. Then 2.0 ml (21.3 mmol) of 2-bromoethyl methyl ether were added. Since the conversion was still incomplete after stirring at RT for 16 h, the mixture was stirred at 50° C. for a further 24 h. After cooling to RT, the mixture was diluted with water and extracted with dichloromethane. The organic phase was washed with water and saturated sodium chloride solution. After drying over anhydrous magnesium sulfate, the mixture was filtered and concentrated. 2.50 g (81% of theory, 81% purity) of the title compound were obtained, which was used for subsequent reactions without further purification.

LC/MS (Method 2, ESIpos): Rₜ=0.84 min, m/z=353 [M+H]⁺.

### Example 92A

### 3-(cis-3-Methoxycyclobutyl)-5-methyl-2,4-dioxo-1-(3,3,3-trifluoropropyl)-1,2,3,4-tetrahydrothieno[2,3-d]pyrimidine-6-carbaldehyde

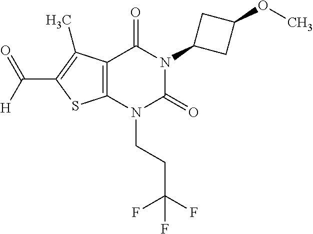 [SMILES: CO[C@H]1C[C@@H](n2c(=O)c3c(C)c(C=O)sc3n(CCC(F)(F)F)c2=O)C1]

To a solution of 1.99 g (5.14 mmol, 76% purity) of the compound from Ex. 41A in 36 ml of DMF were added 2.34 g (16.9 mmol) of potassium carbonate, and the mixture was stirred at RT for 15 min. Then 2.4 ml (20.0 mmol) of 1,1,1-trifluoro-3-iodopropane were added and the reaction mixture was stirred at 50° C. for 16 h. After cooling to RT, the mixture was diluted with water and stirred for 30 min. The precipitate was filtered off, washed with water and dried. 2.26 g (82% of theory) of the title compound were obtained.

LC/MS (Method 2, ESIpos): Rₜ=0.95 min, m/z=391 [M+H]⁺.

### Example 93A

### 3-(cis-3-Methoxycyclobutyl)-1-(2-methoxyethyl)-5-methyl-2,4-dioxo-1,2,3,4-tetrahydrothieno[2,3-d]pyrimidine-6-carbaldehyde

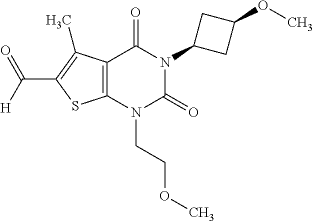 [SMILES: COCCn1c(=O)n([C@H]2C[C@@H](OC)C2)c(=O)c2c(C)c(C=O)sc21]

To a solution of 2.40 g (8.15 mmol, 76% purity) of the compound from Ex. 41A in 43 ml of DMF were added 2.82 g (20.4 mmol) of potassium carbonate, and the mixture was stirred at RT for 15 min. Then 2.4 ml (24.0 mmol) of 2-bromoethyl methyl ether were added and the reaction mixture was stirred at 50° C. for 16 h. After cooling to RT, the mixture was diluted with water and stirred for 30 min. The precipitate was filtered off, washed with water and dried. 2.12 g (64% of theory, 87% purity) of the title compound were obtained, which was used for subsequent reactions without further purification.

LC/MS (Method 2, ESIpos): Rₜ=0.85 min, m/z=353 [M+H]⁺.

### Example 94A

### 3-(3,3-Dimethylcyclobutyl)-5-methyl-2,4-dioxo-1-(3,3,3-trifluoropropyl)-1,2,3,4-tetrahydrothieno[2,3-d]pyrimidine-6-carbaldehyde

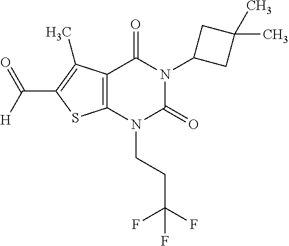 [SMILES: Cc1c(C=O)sc2c1c(=O)n(C1CC(C)(C)C1)c(=O)n2CCC(F)(F)F]

To a solution of 2.40 g (8.21 mmol, 87% purity) of the compound from Ex. 42A in 44 ml of DMF were added 2.84 g (20.5 mmol) of potassium carbonate, and the mixture was stirred at RT for 15 min. Then 2.9 ml (24.6 mmol) of 1,1,1-trifluoro-3-iodopropane were added. Since the conversion was still incomplete after stirring at RT for 3 days, the mixture was stirred at 50° C. for another 20 h. After cooling to RT, the mixture was diluted with water and stirred for 30 min. The precipitated product was filtered off with suction, washed to neutrality with water and dried. 2.83 g (84% of theory, 95% purity) of the title compound were obtained.

LC/MS (Method 1, ESIpos): Rₜ=1.23 min, m/z=389 [M+H]⁺.

### Example 95A

### 3-(3,3-Dimethylcyclobutyl)-1-(2-methoxyethyl)-5-methyl-2,4-dioxo-1,2,3,4-tetrahydrothieno[2,3-d]pyrimidine-6-carbaldehyde

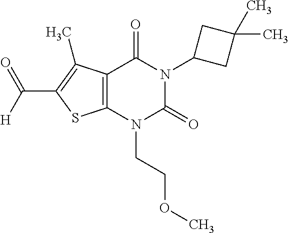 [SMILES: COCCn1c(=O)n(C2CC(C)(C)C2)c(=O)c2c(C)c(C=O)sc21]

To a solution of 2.4 g (8.21 mmol, 87% purity) of the compound from Ex. 42A in 44 ml of DMF were added 2.84 g (20.5 mmol) of potassium carbonate, and the mixture was stirred at RT for 15 min. Then 2.4 ml (24.6 mmol) of 2-bromoethyl methyl ether were added. Since the conversion was still incomplete after stirring at RT for 3 days, the mixture was stirred at 50° C. for another 20 h. After cooling to RT, the mixture was diluted with water and stirred for 30 min. The precipitated product was filtered off with suction, washed to neutrality with water and dried. 2.55 g (81% of theory, 91% purity) of the title compound were obtained, which was used for subsequent reactions without further purification.

LC/MS (Method 1, ESIpos): Rₜ=2.17 min, m/z=351 [M+H]⁺.

### Example 96A

### 5-Methyl-2,4-dioxo-3-(spiro[3.3]hept-2-yl)-1-(3,3,3-trifluoropropyl)-1,2,3,4-tetrahydrothieno[2,3-d]pyrimidine-6-carbaldehyde

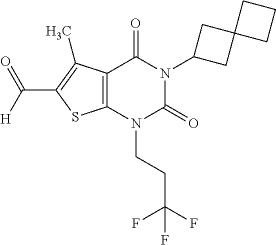 [SMILES: Cc1c(C=O)sc2c1c(=O)n(C1CC3(CCC3)C1)c(=O)n2CCC(F)(F)F]

Analogously to the process described in Ex. 52A, 2.35 g (7.72 mmol) of the compound from Ex. 43A and 2.7 ml (23.2 mmol) of 1,1,1-trifluoro-3-iodopropane were used to prepare 2.70 g (83% of theory, 96% purity) of the title compound.

¹H-NMR (400 MHz, DMSO-d₆, δ/ppm): 10.09 (s, 1H), 5.07-4.93 (m, 1H), 4.12 (t, 2H), 2.86-2.70 (m, 4H), 2.77 (s, 3H), 2.33-2.22 (m, 2H), 2.12-2.03 (m, 2H), 2.02-1.94 (m, 2H), 1.88-1.75 (m, 2H).

LC/MS (Method 1, ESIpos): Rₜ=2.43 min, m/z=401.11 [M+H]⁺.

### Example 97A

### 1-(2-Methoxyethyl)-5-methyl-2,4-dioxo-3-(spiro[3.3]hept-2-yl)-1,2,3,4-tetrahydrothieno[2,3-d]pyrimidine-6-carbaldehyde

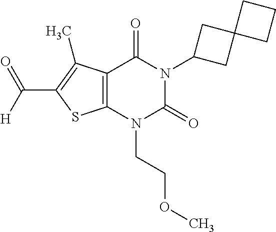 [SMILES: COCCn1c(=O)n(C2CC3(CCC3)C2)c(=O)c2c(C)c(C=O)sc21]

Analogously to the method described in Ex. 45A, 2.35 g (7.72 mmol) of the compound from Ex. 43A and 1.5 ml (15.4 mmol) of 2-bromoethyl methyl ether were used to prepare 2.51 g (89% of theory) of the title compound. The reaction in this case was not effected at RT but at 50° C.

¹H-NMR (400 MHz, DMSO-d₆, δ/ppm): 10.07 (s, 1H), 5.01 (quin, 1H), 4.05 (t, 2H), 3.63 (t, 2H), 3.24 (s, 3H), 2.86-2.72 (m, 2H), 2.75 (s, 3H), 2.27 (td, 2H), 2.07 (t, 2H), 2.02-1.94 (m, 2H), 1.88-1.74 (m, 2H).

LC/MS (Method 1, ESIpos): Rₜ=2.28 min, m/z=363.14 [M+H]⁺.

### Example 98A

### 3-Cyclopentyl-5-methyl-2,4-dioxo-1-(3,3,3-trifluoropropyl)-1,2,3,4-tetrahydrothieno[2,3-d]pyrimidine-6-carbaldehyde

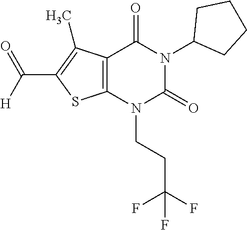 [SMILES: Cc1c(C=O)sc2c1c(=O)n(C1CCCC1)c(=O)n2CCC(F)(F)F]

Analogously to the process described in Ex. 52A, 2.55 g (8.89 mmol, 97% purity) of the compound from Ex. 44A and 3.1 ml (26.7 mmol) of 1,1,1-trifluoro-3-iodopropane were used to prepare 3.30 g (93% of theory, 94% purity) of the title compound.

¹H-NMR (400 MHz, DMSO-d₆, δ/ppm): 10.10 (s, 1H), 5.35-5.22 (m, 1H), 4.15 (t, 2H), 2.87-2.72 (m, 2H), 2.79 (s, 3H), 2.09-1.96 (m, 2H), 1.95-1.83 (m, 2H), 1.82-1.71 (m, 2H), 1.62-1.50 (m, 2H).

LC/MS (Method 2, ESIpos): Rₜ=1.15 min, m/z=375 [M+H]⁺.

### Example 99A

### 3-Cyclopentyl-1-(2-methoxyethyl)-5-methyl-2,4-dioxo-1,2,3,4-tetrahydrothieno[2,3-d]pyrimidine-6-carbaldehyde

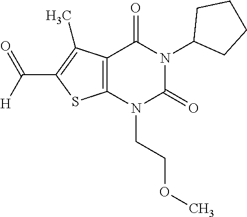 [SMILES: COCCn1c(=O)n(C2CCCC2)c(=O)c2c(C)c(C=O)sc21]

Analogously to the method described in Ex. 52A, 2.55 g (8.89 mmol, 97% purity) of the compound from Ex. 44A and 2.6 ml (27.5 mmol) of 2-bromoethyl methyl ether were used to prepare 2.78 g (88% of theory, 96% purity) of the title compound.

¹H-NMR (400 MHz, DMSO-d₆, δ/ppm): 10.08 (s, 1H), 5.28 (quin, 1H), 4.08 (t, 2H), 3.65 (t, 2H), 3.24 (s, 3H), 2.77 (s, 3H), 2.08-1.96 (m, 2H), 1.95-1.83 (m, 2H), 1.82-1.71 (m, 2H), 1.62-1.49 (m, 2H).

LC/MS (Method 1, ESIpos): Rₜ=1.97 min, m/z=337.12 [M+H]⁺.

### Example 100A

### 6-{[(2-Aminoethyl)amino]methyl}-3-cyclopropyl-1,5-dimethylthieno[2,3-d]pyrimidine-2,4(1H,3H)-dione

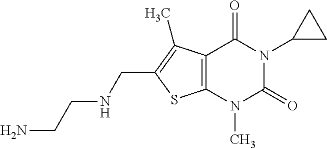 [SMILES: Cc1c(CNCCN)sc2c1c(=O)n(C1CC1)c(=O)n2C]

200 mg (0.757 mmol) of the compound from Ex. 48A were dissolved in a mixture of 5 ml of methanol and 2 ml of dichloromethane. Subsequently, 304 μl (4.54 mmol) of 1,2-diaminoethane and 173 μl (3.03 mmol) of acetic acid were added at RT. After 30 min, 109 mg (3.03 mmol) of sodium cyanoborohydride were added. Then the mixture was stirred at 60° C. for 2 days. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 290 mg (99% of theory, 80% purity) of the title compound, which was used for subsequent reactions without further purification.

LC/MS (Method 8, ESIpos): Rₜ=1.14 min, m/z=307 [M+H-H₂]⁺.

### Example 101A 6-{[(2-Aminoethyl)amino]methyl}-1-butyl-3-cyclopropyl-5-methylthieno[2,3-d]pyrimidine-2,4(1H,3H)-dione

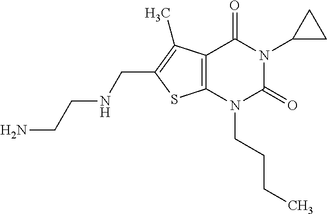 [SMILES: CCCCn1c(=O)n(C2CC2)c(=O)c2c(C)c(CNCCN)sc21]

Analogously to the method described in Ex. 100A, 210 mg (0.685 mmol) of the compound from Ex. 49A, 275 μl (4.11 mmol) of 1,2-diaminoethane and 172 mg (2.74 mmol) of sodium cyanoborohydride were used to prepare 250 mg (98% of theory, 95% purity) of the title compound.

The reaction time here was about 16 h.

LC/MS (Method 1, ESIpos): Rₜ=0.71 min, m/z=291.12 [M+H−C₂H₈N₂]⁺.

### Example 102A

### 6-{[(2-Aminoethyl)amino]methyl}-3-cyclopropyl-1-(3-fluoropropyl)-5-methylthieno[2,3-d]pyrimidine-2,4(1H,3H)-dione

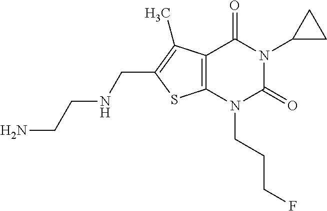 [SMILES: Cc1c(CNCCN)sc2c1c(=O)n(C1CC1)c(=O)n2CCCF]

Analogously to the method described in Ex. 100A, 385 mg (1.24 mmol) of the compound from Ex. 50A and 1,2-diaminoethane were used to prepare 585 mg (94% of theory, 71% purity) of the title compound. The reaction time here was 69 h.

LC/MS (Method 4, ESIpos): Rₜ=0.51 min, m/z=355 [M+H]⁺.

### Example 103A

### 6-{[(2-Aminoethyl)amino]methyl}-3-cyclopropyl-5-methyl-1-(3,3,3-trifluoropropyl)thieno[2,3-d]pyrimidine-2,4(1H,3H)-dione

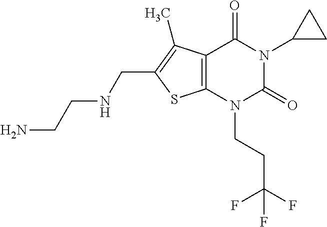 [SMILES: Cc1c(CNCCN)sc2c1c(=O)n(C1CC1)c(=O)n2CCC(F)(F)F]

250 mg (0.722 mmol) of the compound from Ex. 51A were dissolved in a mixture of 5.5 ml of methanol and 2.5 ml of dichloromethane. Subsequently, 290 μl (4.33 mmol) of 1,2-diaminoethane and 165 μl (2.89 mmol) of acetic acid were added at RT. After 30 min, 181 mg (2.89 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 312 mg (77% of theory, 70% purity) of the title compound, which was used for subsequent reactions without further purification.

LC/MS (Method 7, ESIpos): Rₜ=1.21 min, m/z=331 [M+H−C₂H₈N₂]⁺.

### Example 104A

### 6-{[(2-Aminoethyl)amino]methyl}-1-(cyclobutylmethyl)-3-cyclopropyl-5-methylthieno[2,3-d]pyrimidine-2,4(1H,3H)-dione

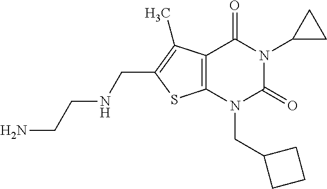 [SMILES: Cc1c(CNCCN)sc2c1c(=O)n(C1CC1)c(=O)n2CC1CCC1]

200 mg (0.628 mmol) of the compound from Ex. 52A were dissolved in a mixture of 5 ml of methanol and 2 ml of dichloromethane. Subsequently, 252 μl (3.77 mmol) of 1,2-diaminoethane and 144 μl (2.51 mmol) of acetic acid were added at RT. After 30 min, 158 mg (2.51 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 240 mg (98% of theory, 93% purity) of the title compound, which was used for subsequent reactions without further purification.

LC/MS (Method 8, ESIpos): Rₜ=1.63 min, m/z=303 [M+H−C₂H₈N₂]⁺.

### Example 105A

### 6-{[(2-Aminoethyl)amino]methyl}-3-cyclopropyl-1-[(2,2-difluorocyclopropyl)methyl]-5-methylthieno[2,3-d]pyrimidine-2,4(1H,3H)-dione (racemate)

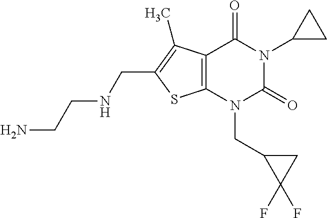 [SMILES: Cc1c(CNCCN)sc2c1c(=O)n(C1CC1)c(=O)n2CC1CC1(F)F]

Analogously to the method described in Ex. 103A, 400 mg (1.17 mmol) of the compound from Ex. 53A, 471 μl (7.05 mmol) of 1,2-diaminoethane and 295 mg (4.70 mmol) of sodium cyanoborohydride were used to prepare 490 mg (99% of theory, 92% purity) of the title compound.

### Example 106A

### 6-{[(2-Aminoethyl)amino]methyl}-3-cyclopropyl-1-(2-methoxyethyl)-5-methylthieno[2,3-d]pyrimidine-2,4(1H,3H)-dione

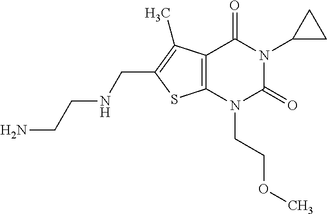 [SMILES: COCCn1c(=O)n(C2CC2)c(=O)c2c(C)c(CNCCN)sc21]

1.0 g (3.24 mmol) of the compound from Ex. 55A were dissolved in a mixture of 25 ml of methanol and 11 ml of dichloromethane. Subsequently, 1.3 ml (19.5 mmol) of 1,2-diaminoethane and 743 μl (13.0 mmol) of acetic acid were added at RT. After 30 min, a further 815 mg (13.0 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 1.23 g (86% of theory, 80% purity) of the title compound, which was used for subsequent reactions without further purification.

LC/MS (Method 2, ESIpos): Rₜ=0.24 min, m/z=293 [M+H−C₂H₈N₂]⁺.

### Example 107A

### 6-{[(2-Aminoethyl)amino]methyl}-3-cyclopropyl-1-(2-ethoxyethyl)-5-methylthieno[2,3-d]pyrimidine-2,4(1H,3H)-dione

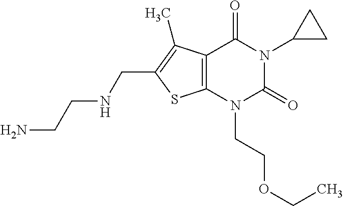 [SMILES: CCOCCn1c(=O)n(C2CC2)c(=O)c2c(C)c(CNCCN)sc21]

Analogously to the method described in Ex. 103A, 200 mg (0.620 mmol) of the compound from Ex. 56A, 249 μl (3.72 mmol) of 1,2-diaminoethane and 156 mg (2.48 mmol) of sodium cyanoborohydride were used to prepare 280 mg (98% of theory, 80% purity) of the title compound.

LC/MS (Method 1, ESIpos): Rₜ=0.58 min, m/z=307.11 [M+H−C₂H₈N₂]⁺.

### Example 108A

### 6-{[(2-Aminoethyl)amino]methyl}-3-cyclopropyl-1-(2-isopropoxyethyl)-5-methylthieno[2,3-d]pyrimidine-2,4(1H,3H)-dione

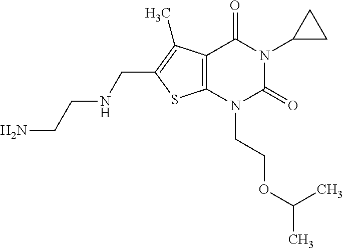 [SMILES: Cc1c(CNCCN)sc2c1c(=O)n(C1CC1)c(=O)n2CCOC(C)C]

Analogously to the method described in Ex. 103A, 200 mg (0.595 mmol) of the compound from Ex. 57A, 238 μl (3.57 mmol) of 1,2-diaminoethane and 149 mg (2.38 mmol) of sodium cyanoborohydride were used to prepare 242 mg (90% of theory, 85% purity) of the title compound.

LC/MS (Method 1, ESIpos): Rₜ=0.61 min, m/z=321.13 [M+H−C₂H₈N₂]⁺.

### Example 109A

### 6-{[(2-Aminoethyl)amino]methyl}-3-cyclopropyl-5-methyl-1-[2-(trifluoromethoxy)ethyl]thieno[2,3-d]pyrimidine-2,4(1H,3H)-dione

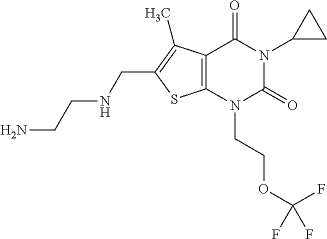 [SMILES: Cc1c(CNCCN)sc2c1c(=O)n(C1CC1)c(=O)n2CCOC(F)(F)F]

240 mg (0.662 mmol) of the compound from Ex. 58A were dissolved in a mixture of 4 ml of methanol and 1.5 ml of dichloromethane. Subsequently, 266 μl (3.97 mmol) of 1,2-diaminoethane and 152 μl (2.65 mmol) of acetic acid were added at RT. After 30 min, 167 mg (2.65 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 330 mg (98% of theory, 80% purity) of the title compound, which was used for subsequent reactions without further purification.

LC/MS (Method 1, ESIpos): Rₜ=0.73 min, m/z=347.07 [M+H−C₂H₈N₂]⁺.

### Example 110A

### 6-{[(2-Aminoethyl)amino]methyl}-3-cyclopropyl-5-methyl-1-(tetrahydrofuran-2-ylmethyl)thieno[2,3-d]pyrimidine-2,4(1H,3H)-dione (racemate)

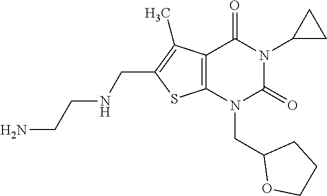 [SMILES: Cc1c(CNCCN)sc2c1c(=O)n(C1CC1)c(=O)n2CC1CCCO1]

400 mg (1.20 mmol) of the compound from Ex. 59A were dissolved in a mixture of 10 ml of methanol and 4 ml of dichloromethane. Subsequently, 480 μl (7.18 mmol) of 1,2-diaminoethane and 274 μl (4.79 mmol) of acetic acid were added at RT. After 30 min, 301 mg (4.79 mmol) of sodium cyanoborohydride were added. Then the mixture was stirred at 50° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 570 mg (100% of theory, 80% purity) of the title compound, which was used for subsequent reactions without further purification.

LC/MS (Method 1, ESIpos): Rₜ=0.56 min, m/z=319.11 [M+H−C₂H₈N₂]⁺.

### Example 111A

### 6-{[(2-Aminoethyl)amino]methyl}-3-cyclopropyl-5-methyl-1-[(2R)-tetrahydrofuran-2-ylmethyl]thieno[2,3-d]pyrimidine-2,4(1H,3H)-dione

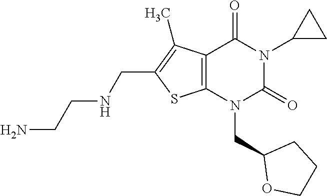 [SMILES: Cc1c(CNCCN)sc2c1c(=O)n(C1CC1)c(=O)n2C[C@H]1CCCO1]

Analogously to the method described in Ex. 100A, 150 mg (0.449 mmol) of the compound from Ex. 60A, 180 μl (2.69 mmol) of 1,2-diaminoethane and 113 mg (1.79 mmol) of sodium cyanoborohydride were used to prepare 170 mg (90% of theory, 90% purity) of the title compound.

LC/MS (Method 1, ESIpos): Rₜ=0.56 min, m/z=319.11 [M+H−C₂H₈N₂]⁺.

### Example 112A

### 6-{[(2-Aminoethyl)amino]methyl}-3-cyclopropyl-5-methyl-1-[(2S)-tetrahydrofuran-2-ylmethyl]thieno[2,3-d]pyrimidine-2,4(1H,3H)-dione

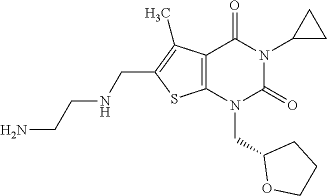 [SMILES: Cc1c(CNCCN)sc2c1c(=O)n(C1CC1)c(=O)n2C[C@@H]1CCCO1]

Analogously to the method described in Ex. 100A, 125 mg (0.374 mmol) of the compound from Ex. 61A, 150 μl (2.24 mmol) of 1,2-diaminoethane and 94 mg (1.50 mmol) of sodium cyanoborohydride were used to prepare 150 mg (95% of theory, 90% purity) of the title compound.

LC/MS (Method 1, ESIpos): Rₜ=0.53 min, m/z=319.11 [M+H−C₂H₈N₂]⁺.

### Example 113A

### 6-{[(2-Aminoethyl)amino]methyl}-1,5-dimethyl-3-(1-methylcyclopropyl)thieno[2,3-d]pyrimidine-2,4(1H,3H)-dione

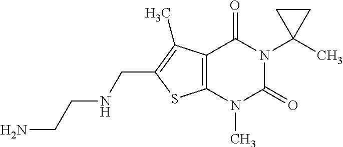 [SMILES: Cc1c(CNCCN)sc2c1c(=O)n(C1(C)CC1)c(=O)n2C]

Analogously to the method described in Ex. 103A, 200 mg (0.719 mmol) of the compound from Ex. 62A, 288 μl (4.31 mmol) of 1,2-diaminoethane and 181 mg (2.87 mmol) of sodium cyanoborohydride were used to prepare 248 mg (96% of theory, 90% purity) of the title compound.

LC/MS (Method 1, ESIpos): Rₜ=0.45 min, m/z=263.08 [M+H−C₂H₈N₂]⁺.

### Example 114A

### 6-{[(2-Aminoethyl)amino]methyl}-1-butyl-5-methyl-3-(1-methylcyclopropyl)thieno[2,3-d]pyrimidine-2,4(1H,3H)-dione

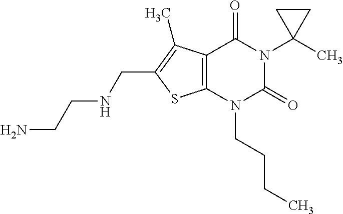 [SMILES: CCCCn1c(=O)n(C2(C)CC2)c(=O)c2c(C)c(CNCCN)sc21]

235 mg (0.733 mmol) of the compound from Ex. 63A were dissolved in a mixture of 5 ml of methanol and 2 ml of dichloromethane. Subsequently, 294 μl (4.40 mmol) of 1,2-diaminoethane and 176 μl (2.93 mmol) of acetic acid were added at RT. After 30 min, 184 mg (2.93 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 298 mg (100% of theory, 90% purity) of the title compound, which was used for subsequent reactions without further purification.

LC/MS (Method 1, ESIpos): Rₜ=0.78 min, m/z=305.13 [M+H−C₂H₈N₂]⁺.

### Example 115A

### 6-{[(2-Aminoethyl)amino]methyl}-5-methyl-3-(1-methylcyclopropyl)-1-(3,3,3-trifluoropropyl)thieno[2,3-d]pyrimidine-2,4(1H,3H)-dione

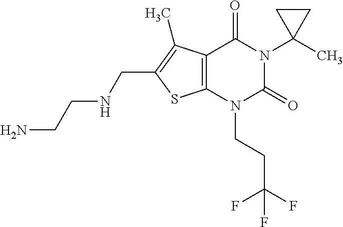 [SMILES: Cc1c(CNCCN)sc2c1c(=O)n(C1(C)CC1)c(=O)n2CCC(F)(F)F]

1.35 g (3.52 mmol, 94% purity) of the compound from Ex. 64A were dissolved in a mixture of 31 ml of methanol and 13 ml of dichloromethane. Then 1.4 ml (21 mmol) of 1,2-diaminoethane and 0.8 ml (14.0 mmol) of acetic acid were added at RT. The mixture was stirred at RT for 30 minutes, and then 886 mg (14.1 mmol) of sodium cyanoborohydride were added. After the reaction mixture had been stirred at 60° C. for 16 h, it was diluted with 14 ml of water, 10 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.30 g (78% of theory, 86% purity) of the title compound, which was used for subsequent reactions without further purification.

LC/MS (Method 2, ESIpos): Rₜ=0.47 min, m/z=405 [M+H]⁺.

### Example 116A

### 6-{[(2-Aminoethyl)amino]methyl}-1-(cyclobutylmethyl)-5-methyl-3-(1-methylcyclopropyl)thieno[2,3-d]pyrimidine-2,4(1H,3H)-dione

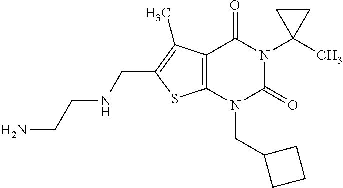 [SMILES: Cc1c(CNCCN)sc2c1c(=O)n(C1(C)CC1)c(=O)n2CC1CCC1]

180 mg (0.541 mmol) of the compound from Ex. 65A were dissolved in a mixture of 3.5 ml of methanol and 1.5 ml of dichloromethane. Subsequently, 217 μl (3.25 mmol) of 1,2-diaminoethane and 124 μl (2.17 mmol) of acetic acid were added at RT. After 30 min, 136 mg (2.17 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 240 mg (100% of theory, 85% purity) of the title compound, which was used for subsequent reactions without further purification.

LC/MS (Method 2, ESIpos): Rₜ=0.56 min, m/z=317 [M+H−C₂H₈N₂]⁺.

### Example 117A

### 6-{[(2-Aminoethyl)amino]methyl}-1-[(2,2-difluorocyclopropyl)methyl]-5-methyl-3-(1-methylcyclopropyl)thieno[2,3-d]pyrimidine-2,4(1H,3H)-dione (racemate)

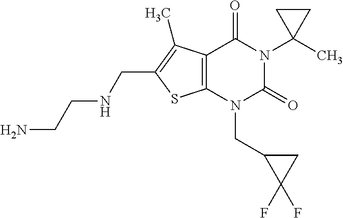 [SMILES: Cc1c(CNCCN)sc2c1c(=O)n(C1(C)CC1)c(=O)n2CC1CC1(F)F]

500 mg (1.41 mmol) of the compound from Ex. 66A were dissolved in a mixture of 10 ml of methanol and 4 ml of dichloromethane. Subsequently, 566 μl (8.47 mmol) of 1,2-diaminoethane and 323 μl (5.64 mmol) of acetic acid were added at RT. After 30 min, 355 mg (5.64 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 568 mg (89% of theory, 89% purity) of the title compound, which was used for subsequent reactions without further purification.

LC/MS (Method 1, ESIpos): Rₜ=0.73 min, m/z=330.10 [M+H−C₂H₈N₂]⁺.

### Example 118A

### 6-{[(2-Aminoethyl)amino]methyl}-1-(2-methoxyethyl)-5-methyl-3-(1-methylcyclopropyl)thieno[2,3-d]pyrimidine-2,4(1H,3H)-dione

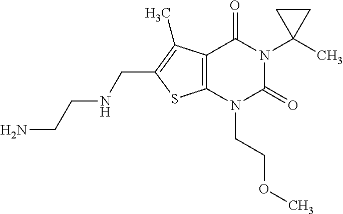 [SMILES: COCCn1c(=O)n(C2(C)CC2)c(=O)c2c(C)c(CNCCN)sc21]

13.2 g (40.9 mmol) of the compound from Ex. 68A were dissolved in a mixture of 300 ml of methanol and 140 ml of dichloromethane. Subsequently, 16.4 ml (246 mmol) of 1,2-diaminoethane and 9.4 ml (164 mmol) of acetic acid were added at RT. After 30 min, 10.3 g (164 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 19.1 g (100% of theory, 79% purity) of the title compound, which was used for subsequent reactions without further purification.

### Example 119A

### 6-{[(2-Aminoethyl)amino]methyl}-5-methyl-3-(1-methylcyclopropyl)-1-[2-(trifluoromethoxy)ethyl]thieno[2,3-d]pyrimidine-2,4(1H,3H)-dione

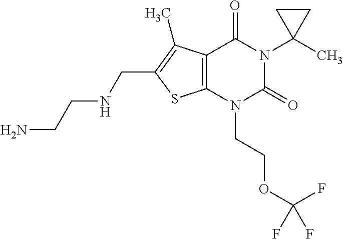 [SMILES: Cc1c(CNCCN)sc2c1c(=O)n(C1(C)CC1)c(=O)n2CCOC(F)(F)F]

300 mg (0.797 mmol) of the compound from Ex. 69A were dissolved in a mixture of 5 ml of methanol and 2 ml of dichloromethane. Subsequently, 320 μl (4.78 mmol) of 1,2-diaminoethane and 183 μl (3.19 mmol) of acetic acid were added at RT. After 30 min, 200 mg (3.19 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 359 mg (99% of theory, 93% purity) of the title compound, which was used for subsequent reactions without further purification.

LC/MS (Method 1, ESIpos): Rₜ=0.81 min, m/z=361.08 [M+H−C₂H₈N₂]⁺.

### Example 120A

### 6-{[(2-Aminoethyl)amino]methyl}-1-(2-ethoxyethyl)-5-methyl-3-(1-methylcyclopropyl)thieno[2,3-d]pyrimidine-2,4(1H,3H)-dione

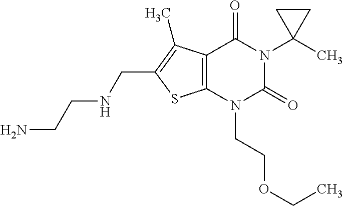 [SMILES: CCOCCn1c(=O)n(C2(C)CC2)c(=O)c2c(C)c(CNCCN)sc21]

Analogously to the method described in Ex. 103A, 195 mg (0.580 mmol) of the compound from Ex. 70A, 233 μl (3.48 mmol) of 1,2-diaminoethane and 146 mg (2.32 mmol) of sodium cyanoborohydride were used to prepare 240 mg (95% of theory, 88% purity) of the title compound.

LC/MS (Method 2, ESIpos): Rₜ=0.43 min, m/z=321 [M+H−C₂H₈N₂]⁺.

### Example 121A

### 6-{[(2-Aminoethyl)amino]methyl}-1-(2-isopropoxyethyl)-5-methyl-3-(1-methylcyclopropyl)thieno[2,3-d]pyrimidine-2,4(1H,3H)-dione

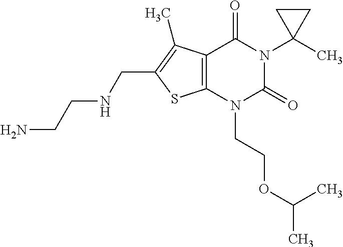 [SMILES: Cc1c(CNCCN)sc2c1c(=O)n(C1(C)CC1)c(=O)n2CCOC(C)C]

Analogously to the method described in Ex. 103A, 200 mg (0.571 mmol) of the compound from Ex. 71A, 229 μl (3.42 mmol) of 1,2-diaminoethane and 143 mg (2.28 mmol) of sodium cyanoborohydride were used to prepare 250 mg (99% of theory, 90% purity) of the title compound.

LC/MS (Method 1, ESIpos): Rₜ=0.76 min, m/z=335.14 [M+H−C₂H₈N₂]⁺.

### Example 122A

### 6-{[(2-Aminoethyl)amino]methyl}-5-methyl-3-(1-methylcyclopropyl)-1-[(2R)-tetrahydrofuran-2-ylmethyl]thieno[2,3-d]pyrimidine-2,4(1H,3H)-dione

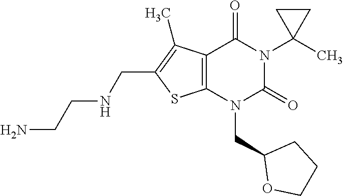 [SMILES: Cc1c(CNCCN)sc2c1c(=O)n(C1(C)CC1)c(=O)n2C[C@H]1CCCO1]

190 mg (0.545 mmol) of the compound from Ex. 72A were dissolved in a mixture of 4 ml of methanol and 1.5 ml of dichloromethane. Subsequently, 219 μl (3.27 mmol) of 1,2-diaminoethane and 125 μl (2.18 mmol) of acetic acid were added at RT. After 30 min, 137 mg (2.18 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 220 mg (97% of theory, 95% purity) of the title compound, which was used for subsequent reactions without further purification.

LC/MS (Method 1, ESIpos): Rₜ=0.63 min, m/z=333.13 [M+H−C₂H₈N₂]⁺.

### Example 123A

### 6-{[(2-Aminoethyl)amino]methyl}-5-methyl-3-(1-methylcyclopropyl)-1-[(2S)-tetrahydrofuran-2-ylmethyl]thieno[2,3-d]pyrimidine-2,4(1H,3H)-dione

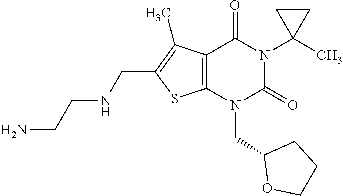 [SMILES: Cc1c(CNCCN)sc2c1c(=O)n(C1(C)CC1)c(=O)n2C[C@@H]1CCCO1]

Analogously to the method described in Ex. 103A, 250 mg (0.718 mmol) of the compound from Ex. 73A, 288 μl (4.31 mmol) of 1,2-diaminoethane and 180 mg (2.87 mmol) of sodium cyanoborohydride were used to prepare 308 mg (98% of theory, 90% purity) of the title compound.

LC/MS (Method 1, ESIpos): Rₜ=0.63 min, m/z=333.13 [M+H−C₂H₈N₂]⁺.

### Example 124A

### 6-{[(2-Aminoethyl)amino]methyl}-1-(2-methoxyethyl)-5-methyl-3-[1-(trifluoromethyl)cyclopropyl]thieno[2,3-d]pyrimidine-2,4(1H,3H)-dione

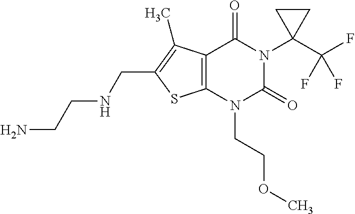 [SMILES: COCCn1c(=O)n(C2(C(F)(F)F)CC2)c(=O)c2c(C)c(CNCCN)sc21]

Analogously to the method described in Ex. 103A, 1.10 g (2.92 mmol) of the compound from Ex. 74A, 1.2 ml (17.5 mmol) of 1,2-diaminoethane and 735 mg (11.7 mmol) of sodium cyanoborohydride were used to prepare 1.28 g (98% of theory, 95% purity) of the title compound.

LC/MS (Method 2, ESIpos): Rₜ=0.44 min, m/z=421 [M+H]⁺.

### Example 125A

### 6-{[(2-Aminoethyl)amino]methyl}-1-(3-fluoropropyl)-5-methyl-3-(2-methylcyclopropyl)thieno[2,3-d]pyrimidine-2,4(1H,3H)-dione (trans racemate)

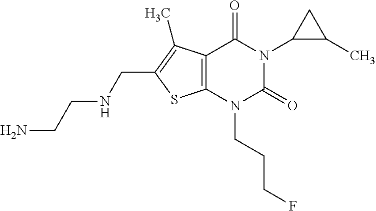 [SMILES: Cc1c(CNCCN)sc2c1c(=O)n(C1CC1C)c(=O)n2CCCF]

430 mg (1.28 mmol) of the compound from Ex. 75A were dissolved in a mixture of 26 ml of methanol and 12.4 ml of dichloromethane. Then 860 μl (12.8 mmol) of 1,2-diaminoethane and 294 μl (5.14 mmol) of acetic acid were added at RT. The mixture was stirred at RT for 30 minutes, and then 340 mg (5.14 mmol) of sodium cyanoborohydride were added. After the reaction mixture had been stirred at 60° C. for 95 h, it was admixed with 100 ml of water (pH about 9) and extracted with ethyl acetate. The organic extract was washed with saturated sodium chloride solution, dried over anhydrous sodium sulfate, filtered and concentrated. The crude product obtained, after drying under high vacuum, gave 510 mg (73% of theory, 68% purity) of the title compound, which was used for subsequent reactions without further purification.

LC/MS (Method 4, ESIpos): Rₜ=0.57 min, m/z=369 [M+H]⁺.

### Example 126A

### 6-{[(2-Aminoethyl)amino]methyl}-5-methyl-3-(2-methylcyclopropyl)-1-(3,3,3-trifluoropropyl)thieno[2,3-d]pyrimidine-2,4(1H,3H)-dione (trans racemate)

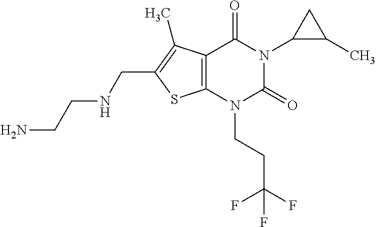 [SMILES: Cc1c(CNCCN)sc2c1c(=O)n(C1CC1C)c(=O)n2CCC(F)(F)F]

487 mg (1.35 mmol) of the compound from Ex. 76A were dissolved in a mixture of 27.4 ml of methanol and 13 ml of dichloromethane. Then 903 μl (13.5 mmol) of 1,2-diaminoethane and 309 μl (5.4 mmol) of acetic acid were added at RT. The mixture was stirred at RT for 30 minutes, and then 357 mg (5.4 mmol) of sodium cyanoborohydride were added. After the reaction mixture had been stirred at 60° C. for 95 h, it was admixed with 100 ml of water (pH about 9) and extracted with ethyl acetate. The organic extract was washed with saturated sodium chloride solution, dried over anhydrous sodium sulfate, filtered and concentrated. The crude product obtained, after drying under high vacuum, gave 610 mg (91% of theory, 82% purity) of the title compound, which was used for subsequent reactions without further purification.

LC/MS (Method 4, ESIpos): Rₜ=0.65 min, m/z=405 [M+H]⁺.

### Example 127A

### 6-{[(2-Aminoethyl)amino]methyl}-1-(2-methoxyethyl)-5-methyl-3-(2-methylcyclopropyl)thieno[2,3-d]pyrimidine-2,4(1H,3H)-dione (trans racemate)

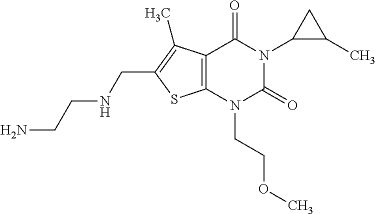 [SMILES: COCCn1c(=O)n(C2CC2C)c(=O)c2c(C)c(CNCCN)sc21]

397 mg (1.23 mmol) of the compound from Ex. 77A were dissolved in a mixture of 25 ml of methanol and 12 ml of dichloromethane. Then 825 μl (12.3 mmol) of 1,2-diaminoethane and 282 μl (4.93 mmol) of acetic acid were added at RT. The mixture was stirred at RT for 30 minutes, and then 326 mg (4.93 mmol) of sodium cyanoborohydride were added. After the reaction mixture had been stirred at 60° C. for 95 h, it was admixed with 100 ml of water (pH about 9) and extracted with ethyl acetate. The organic extract was washed with saturated sodium chloride solution, dried over anhydrous sodium sulfate, filtered and concentrated. The crude product obtained, after drying under high vacuum, gave 504 mg (90% of theory, 81% purity) of the title compound, which was used for subsequent reactions without further purification.

LC/MS (Method 4, ESIpos): Rₜ=0.55 min, m/z=367 [M+H]⁺.

### Example 128A

### 6-{[(2-Aminoethyl)amino]methyl}-3-(2,2-dimethylcyclopropyl)-1-(3-fluoropropyl)-5-methylthieno[2,3-d]pyrimidine-2,4(1H,3H)-dione(racemate)

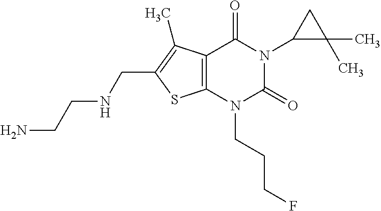 [SMILES: Cc1c(CNCCN)sc2c1c(=O)n(C1CC1(C)C)c(=O)n2CCCF]

Analogously to the method described in Ex. 126A, 566 mg (1.65 mmol) of the compound from Ex. 78A and 1,2-diaminoethane were used to prepare 869 mg (67% of theory, 49% purity) of the title compound. The reaction time here was 81 h.

LC/MS (Method 4, ESIpos): Rₜ=0.64 min, m/z=383 [M+H]⁺.

### Example 129A

### 6-{[(2-Aminoethyl)amino]methyl}-3-(2,2-dimethylcyclopropyl)-5-methyl-1-(3,3,3-trifluoropropyl)thieno[2,3-d]pyrimidine-2,4(1H,3H)-dione (racemate)

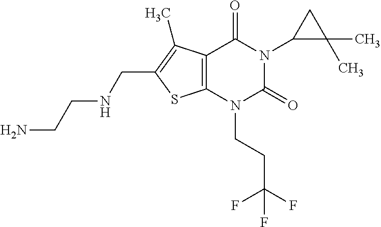 [SMILES: Cc1c(CNCCN)sc2c1c(=O)n(C1CC1(C)C)c(=O)n2CCC(F)(F)F]

557 mg (1.47 mmol) of the compound from Ex. 79A were dissolved in a mixture of 30 ml of methanol and 14 ml of dichloromethane. Then 984 μl (14.7 mmol) of 1,2-diaminoethane and 337 μl (5.89 mmol) of acetic acid were added at RT. The mixture was stirred at RT for 30 minutes, and then 390 mg (5.89 mmol) of sodium cyanoborohydride were added. After the reaction mixture had been stirred at 60° C. for 81 h, it was admixed with 100 ml of water (pH about 9) and extracted with ethyl acetate. The organic extract was washed with saturated sodium chloride solution, dried over anhydrous sodium sulfate, filtered and concentrated. The crude product obtained, after drying under high vacuum, gave 699 mg (95% of theory, 84% purity) of the title compound, which was used for subsequent reactions without further purification.

LC/MS (Method 4, ESIpos): Rₜ=0.70 min, m/z=419 [M+H]⁺.

### Example 130A

### 6-{[(2-Aminoethyl)amino]methyl}-3-(2,2-dimethylcyclopropyl)-1-(2-methoxyethyl)-5-methylthieno[2,3-d]pyrimidine-2,4(1H,3H)-dione(racemate)

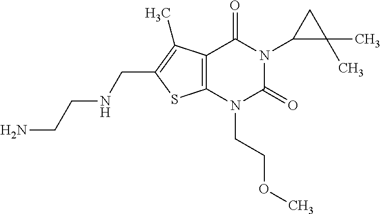 [SMILES: COCCn1c(=O)n(C2CC2(C)C)c(=O)c2c(C)c(CNCCN)sc21]

Analogously to the method described in Ex. 127A, 455 mg (1.29 mmol) of the compound from Ex. 80A and 1,2-diaminoethane were used to prepare 465 mg (84% of theory, 90% purity) of the title compound. The reaction time in this case was 94 h.

LC/MS (Method 4, ESIpos): Rₜ=0.63 min, m/z=381 [M+H]⁺.

### Example 131A

### 6-{[(2-Aminoethyl)amino]methyl}-3-(1-ethylcyclopropyl)-5-methyl-1-(3,3,3-trifluoropropyl)thieno[2,3-d]pyrimidine-2,4(1H,3H)-dione

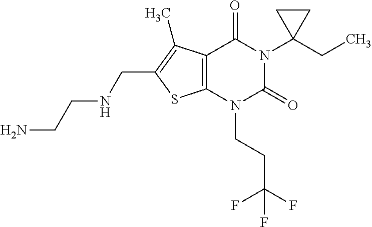 [SMILES: CCC1(n2c(=O)c3c(C)c(CNCCN)sc3n(CCC(F)(F)F)c2=O)CC1]

Analogously to the method described in Ex. 103A, 164 mg (0.428 mmol) of the compound from Ex. 81A, 176 μl (2.63 mmol) of 1,2-diaminoethane and 110 mg (1.75 mmol) of sodium cyanoborohydride were used to prepare 159 mg (78% of theory, 90% purity) of the title compound.

LC/MS (Method 2, ESIpos): Rₜ=0.52 min, m/z=359 [M+H−C₂H₈N₂]⁺.

### Example 132A

### 6-{[(2-Aminoethyl)amino]methyl}-3-(1-ethylcyclopropyl)-1-(2-methoxyethyl)-5-methylthieno[2,3-d]pyrimidine-2,4(1H,3H)-dione

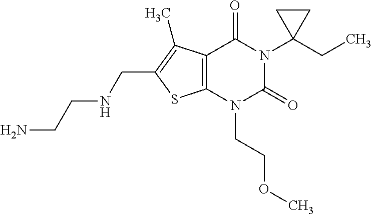 [SMILES: CCC1(n2c(=O)c3c(C)c(CNCCN)sc3n(CCOC)c2=O)CC1]

Analogously to the method described in Ex. 103A, 212 mg (0.616 mmol) of the compound from Ex. 82A, 247 μl (3.70 mmol) of 1,2-diaminoethane and 155 mg (2.47 mmol) of sodium cyanoborohydride were used to prepare 247 mg (94% of theory, 90% purity) of the title compound.

LC/MS (Method 2, ESIpos): Rₜ=0.44 min, m/z=321 [M+H−C₂H₈N₂]⁺.

### Example 133A

### 6-{[(2-Aminoethyl)amino]methyl}-3-cyclobutyl-5-methyl-1-(3,3,3-trifluoropropyl)thieno[2,3-d]pyrimidine-2,4(1H,3H)-dione

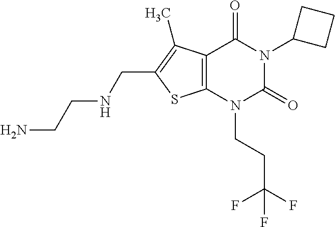 [SMILES: Cc1c(CNCCN)sc2c1c(=O)n(C1CCC1)c(=O)n2CCC(F)(F)F]

2.85 g (7.91 mmol) of the compound from Ex. 83A were dissolved in a mixture of 50 ml of methanol and 25 ml of dichloromethane. Subsequently, 3.2 ml (47.5 mmol) of 1,2-diaminoethane and 1.8 ml (31.6 mmol) of acetic acid were added at RT. After 30 min, 1.99 g (31.6 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 4.0 g (100% of theory, 80% purity) of the title compound, which was used for subsequent reactions without further purification.

LC/MS (Method 2, ESIpos): Rₜ=0.55 min, m/z=405 [M+H]⁺.

### Example 134A

### 6-{[(2-Aminoethyl)amino]methyl}-3-cyclobutyl-1-(2-methoxyethyl)-5-methylthieno[2,3-d]pyrimidine-2,4(1H,3H)-dione

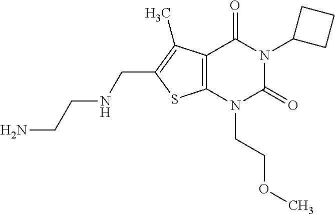 [SMILES: COCCn1c(=O)n(C2CCC2)c(=O)c2c(C)c(CNCCN)sc21]

2.24 g (6.95 mmol) of the compound from Ex. 84A were dissolved in a mixture of 50 ml of methanol and 25 ml of dichloromethane. Subsequently, 2.8 ml (41.7 mmol) of 1,2-diaminoethane and 1.6 ml (27.8 mmol) of acetic acid were added at RT. After 30 min, 1.75 g (27.8 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 2.70 g (84% of theory, 80% purity) of the title compound, which was used for subsequent reactions without further purification.

LC/MS (Method 2, ESIpos): Rₜ=0.45 min, m/z=307 [M+H−C₂H₈N₂]⁺.

### Example 135A

### 6-{[(2-Aminoethyl)amino]methyl}-3-(3,3-difluorocyclobutyl)-5-methyl-1-(3,3,3-trifluoropropyl)thieno[2,3-d]pyrimidine-2,4(1H,3H)-dione

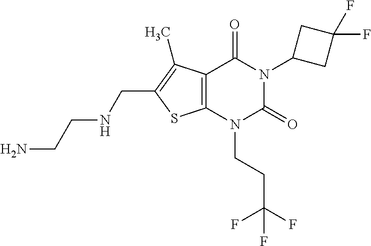 [SMILES: Cc1c(CNCCN)sc2c1c(=O)n(C1CC(F)(F)C1)c(=O)n2CCC(F)(F)F]

1.0 g (2.52 mmol) of the compound from Ex. 85A were dissolved in a mixture of 22 ml of methanol and 9 ml of dichloromethane. Then 1.0 ml (15.1 mmol) of 1,2-diaminoethane and 0.6 ml (10.1 mmol) of acetic acid were added at RT. The mixture was stirred at RT for 30 minutes, and then 634 mg (10.1 mmol) of sodium cyanoborohydride were added. After the reaction mixture had been stirred at 60° C. for 16 h, it was diluted with 10 ml of water, 7.5 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.26 g (98% of theory, 87% purity) of the title compound, which was used for subsequent reactions without further purification.

LC/MS (Method 2, ESIpos): Rₜ=0.59 min, m/z=441 [M+H]⁺.

### Example 136A

### 6-{[(2-Aminoethyl)amino]methyl}-3-(3,3-difluorocyclobutyl)-1-(2-methoxyethyl)-5-methylthieno[2,3-d]pyrimidine-2,4(1H,3H)-dione

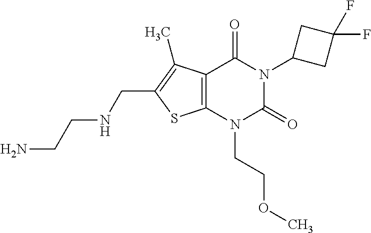 [SMILES: COCCn1c(=O)n(C2CC(F)(F)C2)c(=O)c2c(C)c(CNCCN)sc21]

1.10 g (2.67 mmol, 87% purity) of the compound from Ex. 86A were dissolved in a mixture of 24 ml of methanol and 10 ml of dichloromethane. Then 1.1 ml (16.1 mmol) of 1,2-diaminoethane and 0.6 ml (10.7 mmol) of acetic acid were added at RT. The mixture was stirred at RT for 30 minutes, and then 673 mg (10.70 mmol) of sodium cyanoborohydride were added. After the reaction mixture had been stirred at 60° C. for 16 h, it was diluted with 11 ml of water, 8 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.22 g (78% of theory, 69% purity) of the title compound, which was used for subsequent reactions without further purification.

LC/MS (Method 2, ESIpos): Rₜ=0.59 min, m/z=359 [M+H−C₂H₈N₂]⁺.

### Example 137A

### 6-{[(2-Aminoethyl)amino]methyl}-5-methyl-3-(oxetan-3-yl)-1-(3,3,3-trifluoropropyl)thieno[2,3-d]pyrimidine-2,4(1H,3H)-dione

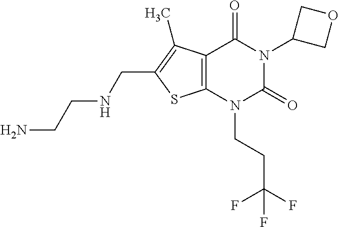 [SMILES: Cc1c(CNCCN)sc2c1c(=O)n(C1COC1)c(=O)n2CCC(F)(F)F]

Analogously to the method described in Ex. 103A, 402 mg (1.03 mmol, 93% purity) of the compound from Ex. 87A, 414 μl (6.19 mmol) of 1,2-diaminoethane and 259 mg (4.13 mmol) of sodium cyanoborohydride were used to prepare 540 mg (100% of theory, 78% purity) of the title compound.

LC/MS (Method 2, ESIpos): Rₜ=0.33 min, m/z=407 [M+H]⁺.

### Example 138A

### 6-{[(2-Aminoethyl)amino]methyl}-5-methyl-3-(1-methylcyclobutyl)-1-(3,3,3-trifluoropropyl)thieno[2,3-d]pyrimidine-2,4(1H,3H)-dione

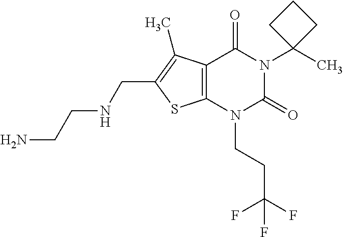 [SMILES: Cc1c(CNCCN)sc2c1c(=O)n(C1(C)CCC1)c(=O)n2CCC(F)(F)F]

Analogously to the method described in Ex. 103A, 1.20 g (3.21 mmol) of the compound from Ex. 88A, 1.3 ml (19.2 mmol) of 1,2-diaminoethane and 806 mg (12.8 mmol) of sodium cyanoborohydride were used to prepare 1.36 g (81% of theory, 80% purity) of the title compound.

LC/MS (Method 2, ESIpos): Rₜ=0.54 min, m/z=359 [M+H−C₂H₈N₂]⁺.

### Example 139A

### 6-{[(2-Aminoethyl)amino]methyl}-1-(2-methoxyethyl)-5-methyl-3-(1-methylcyclobutyl)thieno[2,3-d]pyrimidine-2,4(1H,3H)-dione

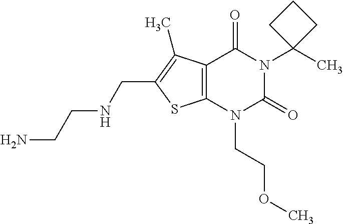 [SMILES: COCCn1c(=O)n(C2(C)CCC2)c(=O)c2c(C)c(CNCCN)sc21]

Analogously to the method described in Ex. 103A, 1.20 g (3.57 mmol) of the compound from Ex. 89A, 1.4 ml (21.4 mmol) of 1,2-diaminoethane and 897 mg (14.3 mmol) of sodium cyanoborohydride were used to prepare 1.30 g (76% of theory, 80% purity) of the title compound.

LC/MS (Method 1, ESIpos): Rₜ=0.72 min, m/z=321.13 [M+H−C₂H₈N₂]⁺.

### Example 140A

### 6-{[(2-Aminoethyl)amino]methyl}-3-(trans-3-methoxycyclobutyl)-5-methyl-1-(3,3,3-trifluoropropyl)thieno[2,3-d]pyrimidine-2,4(1H,3H)-dione

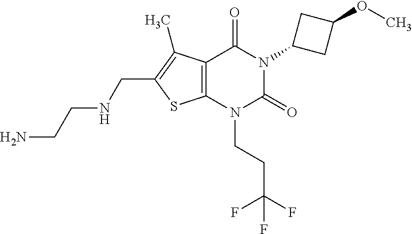 [SMILES: CO[C@H]1C[C@H](n2c(=O)c3c(C)c(CNCCN)sc3n(CCC(F)(F)F)c2=O)C1]

1.60 g (3.80 mmol, 91% purity) of the compound from Ex. 90A were dissolved in a mixture of 34 ml of methanol and 14 ml of dichloromethane. Then 1.5 ml (22.8 mmol) of 1,2-diaminoethane and 0.9 ml (15.2 mmol) of acetic acid were added at RT. The mixture was stirred at RT for 30 minutes, and then 953 mg (15.2 mmol) of sodium cyanoborohydride were added. After the reaction mixture had been stirred at 60° C. for 16 h, it was diluted with 16 ml of water, 12.5 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.66 g (61% of theory, 61% purity) of the title compound, which was used for subsequent reactions without further purification.

LC/MS (Method 2, ESIpos): Rₜ=0.48 min, m/z=375 [M+H−C₂H₈N₂]⁺.

### Example 141A

### 6-{[(2-Aminoethyl)amino]methyl}-3-(trans-3-methoxycyclobutyl)-1-(2-methoxyethyl)-5-methylthieno[2,3-d]pyrimidine-2,4(1H,3H)-dione

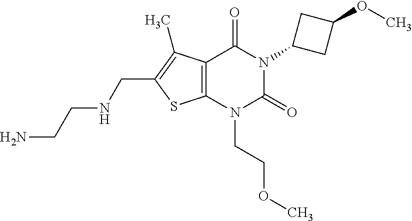 [SMILES: COCCn1c(=O)n([C@H]2C[C@H](OC)C2)c(=O)c2c(C)c(CNCCN)sc21]

1.60 g (3.69 mmol, 81% purity) of the compound from Ex. 91A were dissolved in a mixture of 33 ml of methanol and 14 ml of dichloromethane. Then 1.5 ml (22.1 mmol) of 1,2-diaminoethane and 0.8 ml (14.8 mmol) of acetic acid were added at RT. The mixture was stirred at RT for 30 minutes, and then 928 mg (14.8 mmol) of sodium cyanoborohydride were added. After the reaction mixture had been stirred at 60° C. for 16 h, it was diluted with 16 ml of water, 12.5 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.54 g (67% of theory, 63% purity) of the title compound, which was used for subsequent reactions without further purification.

LC/MS (Method 2, ESIpos): Rₜ=0.39 min, m/z=338 [M+H−C₂H₈N₂]⁺.

### Example 142A

### 6-{[(2-Aminoethyl)amino]methyl}-3-(cis-3-methoxycyclobutyl)-5-methyl-1-(3,3,3-trifluoropropyl)thieno[2,3-d]pyrimidine-2,4(1H,3H)-dione

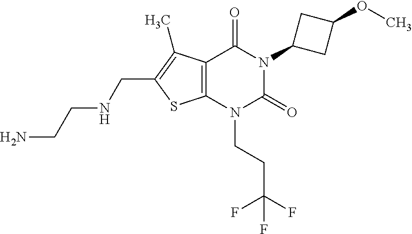 [SMILES: CO[C@H]1C[C@@H](n2c(=O)c3c(C)c(CNCCN)sc3n(CCC(F)(F)F)c2=O)C1]

1.0 g (2.48 mmol) of the compound from Ex. 92A were dissolved in a mixture of 22 ml of methanol and 9.2 ml of dichloromethane. Then 1.0 ml (14.9 mmol) of 1,2-diaminoethane and 0.6 ml (9.94 mmol) of acetic acid were added at RT. The mixture was stirred at RT for 30 minutes, and then 625 mg (9.94 mmol) of sodium cyanoborohydride were added. After the reaction mixture had been stirred at 60° C. for 16 h, it was diluted with 40 ml of water, 7 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.01 g (76% of theory, 81% purity) of the title compound, which was used for subsequent reactions without further purification.

LC/MS (Method 1, ESIpos): Rₜ=0.76 min, m/z=375 [M+H−C₂H₈N₂]⁺.

### Example 143A

### 6-{[(2-Aminoethyl)amino]methyl}-3-(cis-3-methoxycyclobutyl)-1-(2-methoxyethyl)-5-methylthieno[2,3-d]pyrimidine-2,4(1H,3H)-dione

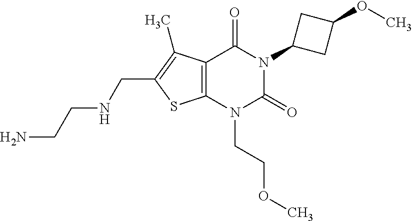 [SMILES: COCCn1c(=O)n([C@H]2C[C@@H](OC)C2)c(=O)c2c(C)c(CNCCN)sc21]

1.38 g (3.41 mmol, 87% purity) of the compound from Ex. 93A were dissolved in a mixture of 30 ml of methanol and 13 ml of dichloromethane. Then 1.4 ml (20.4 mmol) of 1,2-diaminoethane and 13 ml (0.78 mmol) of acetic acid were added at RT. The mixture was stirred at RT for 30 minutes, and then 856 mg (13.6 mmol) of sodium cyanoborohydride were added. After the reaction mixture had been stirred at 60° C. for 16 h, it was diluted with 40 ml of water, 7 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.29 g (95% of theory, 69% purity) of the title compound, which was used for subsequent reactions without further purification.

LC/MS (Method 2, ESIpos): Rₜ=0.56 min, m/z=337 [M+H−C₂H₈N₂]⁺.

### Example 144A

### 6-{[(2-Aminoethyl)amino]methyl}-3-(3,3-dimethylcyclobutyl)-5-methyl-1-(3,3,3-trifluoropropyl)thieno[2,3-d]pyrimidine-2,4(1H,3H)-dione

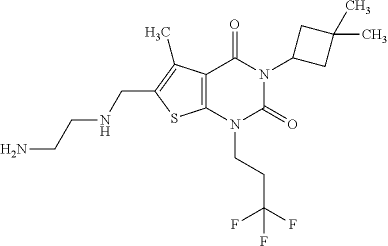 [SMILES: Cc1c(CNCCN)sc2c1c(=O)n(C1CC(C)(C)C1)c(=O)n2CCC(F)(F)F]

1.0 g (2.34 mmol, 95% purity) of the compound from Ex. 94A were dissolved in a mixture of 21 ml of methanol and 9 ml of dichloromethane. Then 0.9 ml (14.1 mmol) of 1,2-diaminoethane and 0.5 ml (9.37 mmol) of acetic acid were added at RT. The mixture was stirred at RT for 30 minutes, and then 589 mg (9.37 mmol) of sodium cyanoborohydride were added. After the reaction mixture had been stirred at 60° C. for 16 h, it was diluted with 40 ml of water, 7 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained, after drying under high vacuum, gave 1.01 g (91% of theory, 92% purity) of the title compound, which was used for subsequent reactions without further purification.

LC/MS (Method 1, ESIpos): Rₜ=1.13 min, m/z=373 [M+H−C₂H₈N₂]⁺.

### Example 145A

### 6-{[(2-Aminoethyl)amino]methyl}-3-(3,3-dimethylcyclobutyl)-1-(2-methoxyethyl)-5-methylthieno[2,3-d]pyrimidine-2,4(1H,3H)-dione

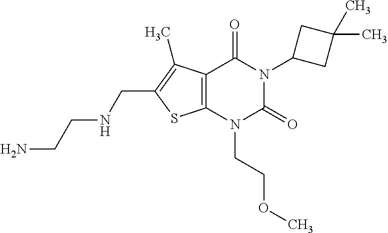 [SMILES: COCCn1c(=O)n(C2CC(C)(C)C2)c(=O)c2c(C)c(CNCCN)sc21]

1.0 g (2.48 mmol, 91% purity) of the compound from Ex. 95A were dissolved in a mixture of 22 ml of methanol and 9 ml of dichloromethane. Then 1.0 ml (14.9 mmol) of 1,2-diaminoethane and 0.6 ml (9.93 mmol) of acetic acid were added at RT. The mixture was stirred at RT for 30 minutes, and then 624 mg (9.93 mmol) of sodium cyanoborohydride were added. After the reaction mixture had been stirred at 60° C. for 16 h, it was diluted with 40 ml of water, 7 ml of 1 M sodium hydroxide solution were added (pH about 9), and extraction was effected with ethyl acetate. The organic extract was washed with saturated sodium chloride solution, dried over anhydrous magnesium sulfate, filtered and concentrated. The crude product obtained was taken up in acetonitrile. The solution was decanted off from the insoluble matter and concentrated, and the residue was dried under high vacuum. This gave 1.78 g (88% of theory, 48% purity) of the title compound, which was used for subsequent reactions without further purification.

LC/MS (Method 2, ESIpos): Rₜ=0.55 min, m/z=395 [M+H]⁺.

### Example 146A

### 6-{[(2-Aminoethyl)amino]methyl}-5-methyl-3-(spiro[3.3]hept-2-yl)-1-(3,3,3-trifluoropropyl)thieno[2,3-d]pyrimidine-2,4(1H,3H)-dione

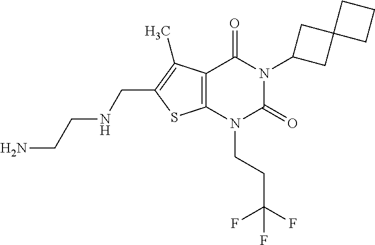 [SMILES: Cc1c(CNCCN)sc2c1c(=O)n(C1CC3(CCC3)C1)c(=O)n2CCC(F)(F)F]

Analogously to the method described in Ex. 103A, 1.20 g (3.0 mmol) of the compound from Ex. 96A, 1.2 ml (18.0 mmol) of 1,2-diaminoethane and 753 mg (12.0 mmol) of sodium cyanoborohydride were used to prepare 1.46 g (89% of theory, 82% purity) of the title compound.

LC/MS (Method 1, ESIpos): Rₜ=1.11 min, m/z=385.12 [M+H−C₂H₈N₂]⁺.

### Example 147A

### 6-{[(2-Aminoethyl)amino]methyl}-1-(2-methoxyethyl)-5-methyl-3-(spiro[3.3]hept-2-yl)thieno[2,3-d]pyrimidine-2,4(1H,3H)-dione

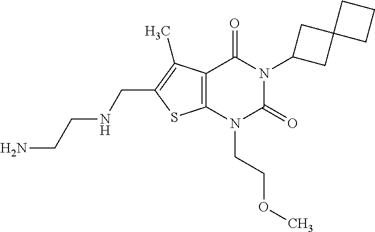 [SMILES: COCCn1c(=O)n(C2CC3(CCC3)C2)c(=O)c2c(C)c(CNCCN)sc21]

Analogously to the method described in Ex. 103A, 1.20 g (3.31 mmol) of the compound from Ex. 97A, 1.3 ml (19.9 mmol) of 1,2-diaminoethane and 832 mg (13.2 mmol) of sodium cyanoborohydride were used to prepare 1.38 g (83% of theory, 81% purity) of the title compound.

LC/MS (Method 1, ESIpos): Rₜ=0.89 min, m/z=347.14 [M+H−C₂H₈N₂]⁺.

### Example 148A

### 6-{[(2-Aminoethyl)amino]methyl}-3-cyclopentyl-5-methyl-1-(3,3,3-trifluoropropyl)thieno[2,3-d]pyrimidine-2,4(1H,3H)-dione

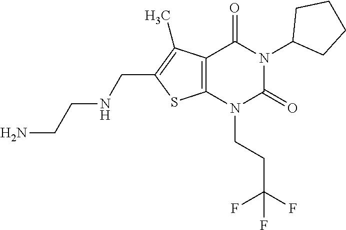 [SMILES: Cc1c(CNCCN)sc2c1c(=O)n(C1CCCC1)c(=O)n2CCC(F)(F)F]

1.60 g (4.27 mmol) of the compound from Ex. 98A were dissolved in a mixture of 29 ml of methanol and 15 ml of dichloromethane. Subsequently, 1.7 ml (25.6 mmol) of 1,2-diaminoethane and 979 μl (17.1 mmol) of acetic acid were added at RT. After 30 min, a further 1.07 g (17.1 mmol) of sodium cyanoborohydride were added. Then the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 1.0 g (31% of theory, 56% purity) of the title compound, which was used for subsequent reactions without further purification.

LC/MS (Method 2, ESIpos): Rₜ=0.56 min, m/z=419 [M+H]⁺.

### Example 149A

### 6-{[(2-Aminoethyl)amino]methyl}-3-cyclopenty-1-(2-methoxyethyl)-5-methylthieno[2,3-d]pyrimidine-2,4(1H,3H)-dione

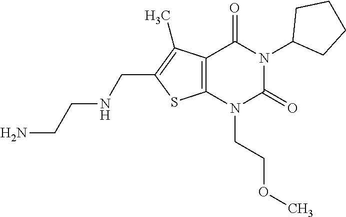 [SMILES: COCCn1c(=O)n(C2CCCC2)c(=O)c2c(C)c(CNCCN)sc21]

Analogously to the method described in Ex. 103A, 1.60 g (4.76 mmol) of the compound from Ex. 99A, 1.9 ml (28.5 mmol) of 1,2-diaminoethane and 1.20 g (19.0 mmol) of sodium cyanoborohydride were used to prepare 1.84 g (83% of theory, 82% purity) of the title compound.

LC/MS (Method 2, ESIpos): Rₜ=0.44 min, m/z=321 [M+H−C₂H₈N₂]⁺.

### Example 150A

### 3-Cyclopropyl-6-{[(2,2-dimethoxyethyl)amino]methyl}-5-methyl-1-(3,3,3-trifluoropropyl)thieno[2,3-d]pyrimidine-2,4(1H,3H)-dione

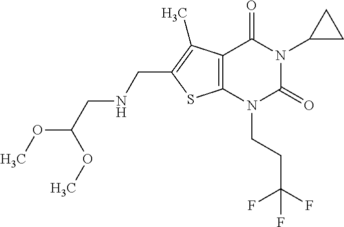 [SMILES: COC(CNCc1sc2c(c1C)c(=O)n(C1CC1)c(=O)n2CCC(F)(F)F)OC]

1.03 g (2.87 mmol) of the compound from Ex. 51A were dissolved in 65 ml of dichloromethane, and 0.46 ml (4.31 mmol) of 2,2-dimethoxyethanamine were added. The mixture was heated to 35° C. for 1 h. After cooling to RT, 1.83 g (8.62 mmol) of sodium triacetoxyborohydride were added. Stirring of the mixture was continued at RT. After 18 h, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. The crude product was purified by preparative HPLC (Method 13). After combination of the product fractions, concentration and drying under high vacuum, 795 mg (63% of theory) of the title compound were obtained.

LC/MS (Method 1, ESIpos): Rₜ=0.97 min, m/z=436 [M+H]⁺.

### Example 151A

### 3-Cyclopropyl-6-{[(2,2-dimethoxyethyl)amino]methyl}-1-(2-methoxyethyl)-5-methylthieno[2,3-d]pyrimidine-2,4(1H,3H)-dione

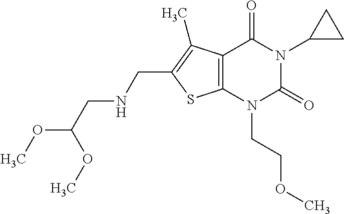 [SMILES: COCCn1c(=O)n(C2CC2)c(=O)c2c(C)c(CNCC(OC)OC)sc21]

888 g (2.50 mmol) of the compound from Ex. 55A were dissolved in 57 ml of dichloromethane, and 0.41 ml (3.76 mmol) of 2,2-dimethoxyethanamine were added. The mixture was heated to 35° C. for 1 h. After cooling to RT, 1.60 g (7.52 mmol) of sodium triacetoxyborohydride were added. Stirring of the mixture was continued at RT. After 18 h, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. 1.10 g (93% of theory, 85% purity) of the title compound were obtained, which was used for subsequent reactions without further purification.

LC/MS (Method 2, ESIpos): Rₜ=0.78 min, m/z=294 [M+H−C₄H₁₁NO₂]⁺.

### Example 152A

### 3-Cyclopropyl-6-{[(2,2-dimethoxyethyl)amino]methyl)aminomethyl}-5-methyl-1-(tetrahydrofuran-2-ylmethyl)thieno[2,3-d]pyrimidine-2,4(1H,3H)-dione (racemate)

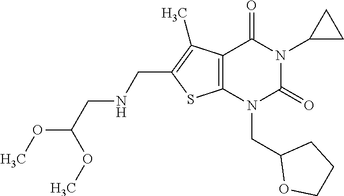 [SMILES: COC(CNCc1sc2c(c1C)c(=O)n(C1CC1)c(=O)n2CC1CCCO1)OC]

1.07 g (2.82 mmol) of the compound from Ex. 59A were dissolved in 63 ml of dichloromethane, and 0.5 ml (4.24 mmol) of 2,2-dimethoxyethanamine were added. The mixture was heated to 35° C. for 1 h. After cooling to RT, 1.80 g (8.47 mmol) of sodium triacetoxyborohydride were added. Stirring of the mixture was continued at RT. After 40 h, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. 1.24 g (62% of theory, 60% purity) of the title compound were obtained, which was used for subsequent reactions without further purification.

LC/MS (Method 7, ESIpos): Rₜ=1.41 min, m/z=320 [M+H−C₄H₁₁NO₂].

### Example 153A

### 1-{[3-Cyclopropyl-5-methyl-2,4-dioxo-1-(3,3,3-trifluoropropyl)-1,2,3,4-tetrahydrothieno[2,3-d]pyrimidin-6-yl]methyl}-1-(2,2-dimethoxyethyl)urea

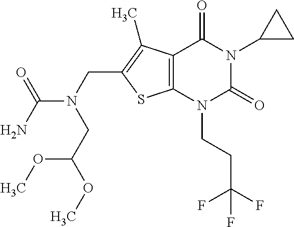 [SMILES: COC(CN(Cc1sc2c(c1C)c(=O)n(C1CC1)c(=O)n2CCC(F)(F)F)C(N)=O)OC]

To a solution of 795 mg (1.83 mmol) of the compound from Ex. 150A in 19 ml of methanol were added, at RT, first 341 mg (4.20 mmol) of potassium cyanate and then 268 μl (3.10 mmol) of perchloric acid (70% in water). After 1 h, the reaction mixture was admixed with water and with aqueous sodium hydrogencarbonate solution and then extracted with ethyl acetate. The organic extract was washed successively with aqueous sodium hydrogencarbonate solution and saturated sodium chloride solution, dried over anhydrous magnesium sulfate, filtered and concentrated. After the residue had been dried under high vacuum, 631 mg (51% of theory, 71% purity) of the title compound were obtained, which was used for subsequent reactions without further purification.

LC/MS (Method 1, ESIpos): Rₜ=1.44 min, m/z=332 [M+H−C₅H₁₂N₂O₃]⁺.

### Example 154A

### 1-{[3-Cyclopropyl-1-(2-methoxyethyl)-5-methyl-2,4-dioxo-1,2,3,4-tetrahydrothieno[2,3-d]pyrimidin-6-yl]methyl}-1-(2,2-dimethoxyethyl)urea

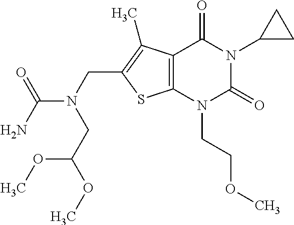 [SMILES: COCCn1c(=O)n(C2CC2)c(=O)c2c(C)c(CN(CC(OC)OC)C(N)=O)sc21]

To a solution of 1.10 g (2.35 mmol, 85% purity) of the compound from Ex. 151A in 24 ml of methanol were added, at RT, first 439 mg (5.41 mmol) of potassium cyanate and then 345 μl (4.0 mmol) of perchloric acid (70% in water). After 1 h, the reaction mixture was admixed with water and with aqueous sodium hydrogencarbonate solution and then extracted with ethyl acetate. The organic extract was washed successively with aqueous sodium hydrogencarbonate solution and saturated sodium chloride solution, dried over anhydrous magnesium sulfate, filtered and concentrated. After the residue had been dried under high vacuum, 1.06 g (67% of theory, 66% purity) of the title compound were obtained, which was used for subsequent reactions without further purification.

LC/MS (Method 1, ESIpos): Rₜ=1.18 min, m/z=293 [M+H−C₅H₁₂N₂O₃]⁺.

### Example 155A

### 1-{[3-Cyclopropyl-5-methyl-2,4-dioxo-1-(tetrahydrofuran-2-ylmethyl)-1,2,3,4-tetrahydrothieno[2,3-d]pyrimidin-6-yl]methyl}-1-(2,2-dimethoxyethyl)urea (racemate)

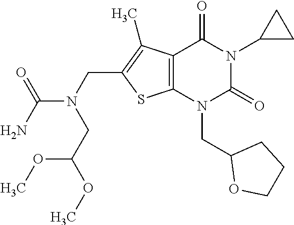 [SMILES: COC(CN(Cc1sc2c(c1C)c(=O)n(C1CC1)c(=O)n2CC1CCCO1)C(N)=O)OC]

To a solution of 1.22 g (1.73 mmol, 60% purity) of the compound from Ex. 152A in 18 ml of methanol were added, at RT, first 324 mg (3.99 mmol) of potassium cyanate and then 254 μl (2.95 mmol) of perchloric acid (70% in water). After 2 h, the reaction mixture was admixed with water and with aqueous sodium hydrogencarbonate solution and then extracted with ethyl acetate. The organic extract was washed successively with aqueous sodium hydrogencarbonate solution and saturated sodium chloride solution, dried over anhydrous magnesium sulfate, filtered and concentrated. After the residue had been dried under high vacuum, 1.07 g (79% of theory, 60% purity) of the title compound were obtained, which was used for subsequent reactions without further purification.

### Example 156A

### tert-Butyl 2-[(3-cyclopropyl-1,5-dimethyl-2,4-dioxo-1,2,3,4-tetrahydrothieno[2,3-d]pyrimidin-6-yl)methylene]hydrazinecarboxylate

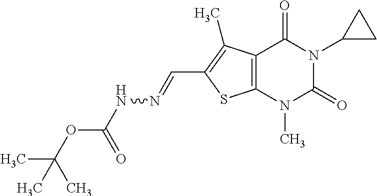 [SMILES: Cc1c(C=NNC(=O)OC(C)(C)C)sc2c1c(=O)n(C1CC1)c(=O)n2C]

To a solution of 235 mg (0.889 mmol) of the compound from Ex. 48A in 10 ml of ethanol were added first 176 mg (1.33 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 328 mg (97% of theory) of the title compound were obtained.

¹H-NMR (400 MHz, DMSO-d₆, δ/ppm): 10.82 (br. s, 1H), 8.28 (s, 1H), 3.42 (s, 3H), 2.63-2.56 (m, 1H), 2.44 (s, 3H), 1.45 (s, 9H), 1.06-0.95 (m, 2H), 0.73-0.63 (m, 2H).

LC/MS (Method 1, ESIneg): Rₜ=1.68 min, m/z=377.13 [M−H]⁻.

### Example 157A

### tert-Butyl 2-[(1-butyl-3-cyclopropyl-5-methyl-2,4-dioxo-1,2,3,4-tetrahydrothieno[2,3-d]pyrimidin-6-yl)methylene]hydrazinecarboxylate

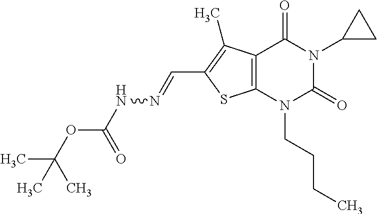 [SMILES: CCCCn1c(=O)n(C2CC2)c(=O)c2c(C)c(C=NNC(=O)OC(C)(C)C)sc21]

Analogously to the method described in Ex. 156A, 300 mg (0.979 mmol) of the compound from Ex. 49A and 194 mg (1.47 mmol) of tert-butyl hydrazinecarboxylate were used to prepare 406 mg (93% of theory, 95% purity) of the title compound.

¹H-NMR (400 MHz, DMSO-d₆, δ/ppm): 10.84 (br. s, 1H), 8.28 (s, 1H), 3.86 (br. t, 2H), 2.63-2.56 (m, 1H), 2.43 (s, 3H), 1.66 (quin, 2H), 1.45 (s, 9H), 1.36 (dq, 2H), 1.04-0.96 (m, 2H), 0.92 (t, 3H), 0.73-0.63 (m, 2H).

LC/MS (Method 2, ESIneg): Rₜ=1.10 min, m/z=419 [M−H]⁻.

### Example 158A

### tert-Butyl 2-{[3-cyclopropyl-5-methyl-2,4-dioxo-1-(3,3,3-trifluoropropyl)-1,2,3,4-tetrahydrothieno[2,3-d]pyrimidin-6-yl]methylene}hydrazinecarboxylate

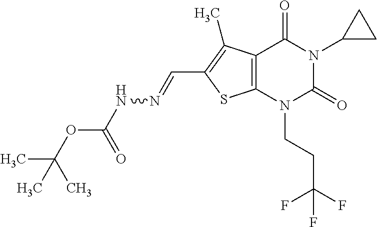 [SMILES: Cc1c(C=NNC(=O)OC(C)(C)C)sc2c1c(=O)n(C1CC1)c(=O)n2CCC(F)(F)F]

To a solution of 500 mg (1.44 mmol) of the compound from Ex. 51A in 13 ml of ethanol were added first 286 mg (2.17 mmol) of tert-butyl hydrazinecarboxylate and then 2 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 663 mg (99% of theory) of the title compound were obtained.

¹H-NMR (400 MHz, DMSO-d₆, δ/ppm): 10.86 (br. s, 1H), 8.29 (s, 1H), 4.10 (t, 2H), 2.85-2.69 (m, 2H), 2.65-2.56 (m, 1H), 2.44 (s, 3H), 1.45 (s, 9H), 1.06-0.96 (m, 2H), 0.73-0.64 (m, 2H).

LC/MS (Method 1, ESIneg): Rₜ=1.97 min, m/z=459.13 [M−H]⁻.

### Example 159A

### tert-Butyl 2-{[1-(cyclobutylmethyl)-3-cyclopropyl-5-methyl-2,4-dioxo-1,2,3,4-tetrahydrothieno[2,3-d]pyrimidin-6-yl]methylene}hydrazinecarboxylate

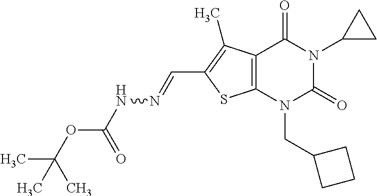 [SMILES: Cc1c(C=NNC(=O)OC(C)(C)C)sc2c1c(=O)n(C1CC1)c(=O)n2CC1CCC1]

Analogously to the method described in Ex. 156A, 284 mg (0.892 mmol) of the compound from Ex. 52A and 177 mg (1.34 mmol) of tert-butyl hydrazinecarboxylate were used to prepare 350 mg (90% of theory) of the title compound.

¹H-NMR (400 MHz, DMSO-d₆, δ/ppm): 10.84 (br. s, 1H), 8.28 (s, 1H), 3.93 (d, 2H), 2.83-2.69 (m, 1H), 2.60 (tt, 1H), 2.43 (s, 3H), 2.04-1.92 (m, 2H), 1.88-1.75 (m, 4H), 1.45 (s, 9H), 1.06-0.94 (m, 2H), 0.73-0.60 (m, 2H).

LC/MS (Method 1, ESIneg): Rₜ=2.15 min, m/z=431.18 [M−H]⁻.

### Example 160A

### tert-Butyl 2-({3-cyclopropyl-1-[(2,2-difluorocyclopropyl)methyl]-5-methyl-2,4-dioxo-1,2,3,4-tetrahydrothieno[2,3-d]pyrimidin-6-yl}methylene)hydrazinecarboxylate (racemate)

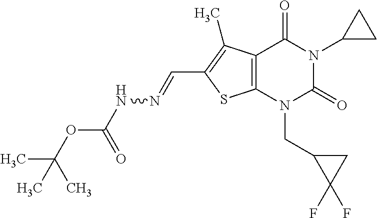 [SMILES: Cc1c(C=NNC(=O)OC(C)(C)C)sc2c1c(=O)n(C1CC1)c(=O)n2CC1CC1(F)F]

Analogously to the method described in Ex. 156A, 575 mg (1.69 mmol) of the compound from Ex. 53A and 335 mg (2.53 mmol) of tert-butyl hydrazinecarboxylate were used to prepare 743 mg (96% of theory) of the title compound.

¹H-NMR (400 MHz, DMSO-d₆, δ/ppm): 10.84 (br. s, 1H), 8.29 (s, 1H), 4.13 (ddd, 1H), 3.95 (dd, 1H), 2.61 (tt, 1H), 2.44 (s, 3H), 2.29-2.13 (m, 1H), 1.77-1.64 (m, 1H), 1.54-1.37 (m, 1H), 1.45 (s, 9H), 1.09-0.92 (m, 2H), 0.78-0.62 (m, 2H).

LC/MS (Method 1, ESIneg): Rₜ=1.98 min, m/z=453.14 [M−H]⁻.

### Example 161A

### tert-Butyl 2-{[1-(cyanomethyl)-3-cyclopropyl-5-methyl-2,4-dioxo-1,2,3,4-tetrahydrothieno[2,3-d]pyrimidin-6-yl]methylene}hydrazinecarboxylate

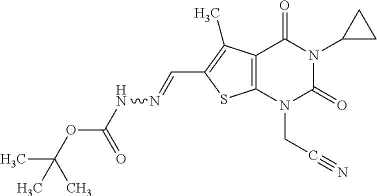 [SMILES: Cc1c(C=NNC(=O)OC(C)(C)C)sc2c1c(=O)n(C1CC1)c(=O)n2CC#N]

Analogously to the method described in Ex. 156A, 235 mg (0.796 mmol) of the compound from Ex. 54A and 158 mg (1.19 mmol) of tert-butyl hydrazinecarboxylate were used to prepare 308 mg (95% of theory) of the title compound.

¹H-NMR (400 MHz, DMSO-d₆, δ/ppm): 10.89 (br. s, 1H), 8.30 (s, 1H), 5.15 (s, 2H), 2.63 (tt, 1H), 2.45 (s, 3H), 1.46 (s, 9H), 1.10-0.92 (m, 2H), 0.81-0.63 (m, 2H).

LC/MS (Method 2, ESIneg): Rₜ=0.89 min, m/z=402 [M−H]⁻.

### Example 162A

### tert-Butyl 2-{[3-cyclopropyl-1-(2-methoxyethyl)-5-methyl-2,4-dioxo-1,2,3,4-tetrahydrothieno[2,3-d]pyrimidin-6-yl]methylene}hydrazinecarboxylate

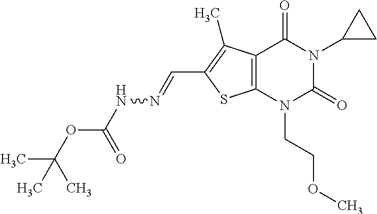 [SMILES: COCCn1c(=O)n(C2CC2)c(=O)c2c(C)c(C=NNC(=O)OC(C)(C)C)sc21]

Analogously to the method described in Ex. 156A, 383 mg (1.16 mmol, 93% purity) of the compound from Ex. 55A and 229 mg (1.73 mmol) of tert-butyl hydrazinecarboxylate were used to prepare 498 mg (98% of theory, 97% purity) of the title compound.

¹H-NMR (400 MHz, DMSO-d₆, δ/ppm): 10.83 (br. s, 1H), 8.27 (s, 1H), 4.11-3.95 (m, 2H), 3.63 (t, 2H), 3.25 (s, 3H), 2.65-2.56 (m, 1H), 2.43 (s, 3H), 1.45 (s, 9H), 1.07-0.94 (m, 2H), 0.75-0.63 (m, 2H).

LC/MS (Method 1, ESIneg): Rₜ=1.72 min, m/z=421.16 [M−H]⁻.

### Example 163A

### tert-Butyl 2-{[3-cyclopropyl-1-(2-ethoxyethyl)-5-methyl-2,4-dioxo-1,2,3,4-tetrahydrothieno[2,3-d]pyrimidin-6-yl]methylene}hydrazinecarboxylate

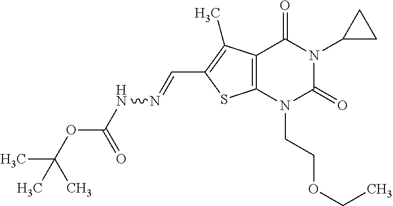 [SMILES: CCOCCn1c(=O)n(C2CC2)c(=O)c2c(C)c(C=NNC(=O)OC(C)(C)C)sc21]

Analogously to the method described in Ex. 156A, 250 mg (0.775 mmol) of the compound from Ex. 56A and 154 mg (1.16 mmol) of tert-butyl hydrazinecarboxylate were used to prepare 328 mg (96% of theory) of the title compound.

¹H-NMR (400 MHz, DMSO-d₆, δ/ppm): 10.83 (br. s, 1H), 8.28 (s, 1H), 4.02 (t, 2H), 3.66 (t, 2H), 3.45 (q, 2H), 2.64-2.56 (m, 1H), 2.43 (s, 3H), 1.45 (s, 9H), 1.05 (t, 3H), 1.03-0.95 (m, 2H), 0.75-0.62 (m, 2H).

LC/MS (Method 1, ESIneg): Rₜ=1.87 min, m/z=435.17 [M−H]⁻.

### Example 164A

### tert-Butyl 2-{[3-cyclopropyl-1-(2-isopropoxyethyl)-5-methyl-2,4-dioxo-1,2,3,4-tetrahydrothieno[2,3-d]pyrimidin-6-yl]methylene}hydrazinecarboxylate

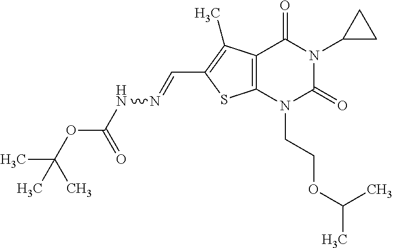 [SMILES: Cc1c(C=NNC(=O)OC(C)(C)C)sc2c1c(=O)n(C1CC1)c(=O)n2CCOC(C)C]

Analogously to the method described in Ex. 156A, 305 mg (0.907 mmol) of the compound from Ex. 57A and 180 mg (1.36 mmol) of tert-butyl hydrazinecarboxylate were used to prepare 381 mg (93% of theory) of the title compound.

¹H-NMR (400 MHz, DMSO-d₆, δ/ppm): 10.82 (br. s, 1H), 8.28 (s, 1H), 3.99 (t, 2H), 3.65 (t, 2H), 3.56 (sept, 1H), 2.65-2.56 (m, 1H), 2.43 (s, 3H), 1.45 (s, 9H), 1.06-0.96 (m, 2H), 1.02 (d, 6H), 0.72-0.63 (m, 2H).

LC/MS (Method 1, ESIneg): Rₜ=1.98 min, m/z=449.19 [M−H]⁻.

### Example 165A

### tert-Butyl 2-({3-cyclopropyl-5-methyl-2,4-dioxo-1-[2-(trifluoromethoxy)ethyl]-1,2,3,4-tetrahydrothieno[2,3-d]pyrimidin-6-yl}methylene)hydrazinecarboxylate

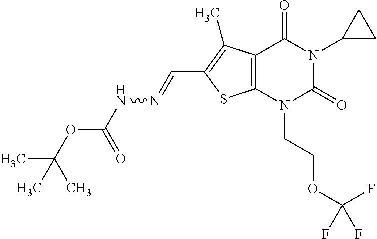 [SMILES: Cc1c(C=NNC(=O)OC(C)(C)C)sc2c1c(=O)n(C1CC1)c(=O)n2CCOC(F)(F)F]

To a solution of 350 mg (0.966 mmol) of the compound from Ex. 58A in 10 ml of ethanol were added first 191 mg (1.45 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 442 mg (96% of theory) of the title compound were obtained.

¹H-NMR (400 MHz, DMSO-d₆, δ/ppm): 10.85 (br. s, 1H), 8.28 (s, 1H), 4.40 (t, 2H), 4.24-4.17 (m, 2H), 2.65-2.57 (m, 1H), 2.44 (s, 3H), 1.45 (s, 9H), 1.07-0.97 (m, 2H), 0.73-0.65 (m, 2H).

LC/MS (Method 1, ESIneg): Rₜ=2.00 min, m/z=475.13 [M−H]⁻.

### Example 166A

### tert-Butyl 2-{[3-cyclopropyl-5-methyl-2,4-dioxo-1-(tetrahydrofuran-2-ylmethyl)-1,2,3,4-tetrahydrothieno[2,3-d]pyrimidin-6-yl]methylene}hydrazinecarboxylate (racemate)

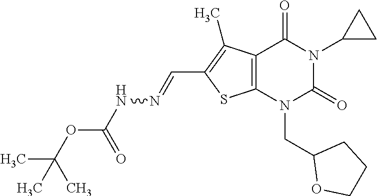 [SMILES: Cc1c(C=NNC(=O)OC(C)(C)C)sc2c1c(=O)n(C1CC1)c(=O)n2CC1CCCO1]

Analogously to the method described in Ex. 156A, 722 mg (2.01 mmol, 93% purity) of the compound from Ex. 59A and 398 mg (3.01 mmol) of tert-butyl hydrazinecarboxylate were used to prepare 861 mg (95% of theory) of the title compound.

¹H-NMR (400 MHz, DMSO-d₆, δ/ppm): 10.83 (br. s, 1H), 8.28 (s, 1H), 4.27-4.15 (m, 1H), 4.06 (dd, 1H), 3.83-3.68 (m, 2H), 3.66-3.57 (m, 1H), 2.65-2.56 (m, 1H), 2.43 (s, 3H), 2.06-1.75 (m, 3H), 1.72-1.59 (m, 1H), 1.45 (s, 9H), 1.06-0.95 (m, 2H), 0.75-0.62 (m, 2H).

LC/MS (Method 1, ESIneg): Rₜ=1.84 min, m/z=447.17 [M−H]⁻.

### Example 167A

### tert-Butyl 2-({3-cyclopropyl-5-methyl-2,4-dioxo-1-[(2R)-tetrahydrofuran-2-ylmethyl]-1,2,3,4-tetrahydrothieno[2,3-d]pyrimidin-6-yl}methylene)hydrazinecarboxylate

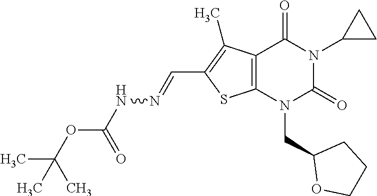 [SMILES: Cc1c(C=NNC(=O)OC(C)(C)C)sc2c1c(=O)n(C1CC1)c(=O)n2C[C@H]1CCCO1]

Analogously to the method described in Ex. 156A, 230 mg (0.688 mmol) of the compound from Ex. 60A and 137 mg (1.03 mmol) of tert-butyl hydrazinecarboxylate were used to prepare 278 mg (90% of theory) of the title compound.

¹H-NMR (400 MHz, DMSO-d₆, δ/ppm): 10.83 (br. s, 1H), 8.28 (s, 1H), 4.27-4.14 (m, 1H), 4.06 (dd, 1H), 3.82-3.67 (m, 2H), 3.67-3.57 (m, 1H), 2.64-2.57 (m, 1H), 2.43 (s, 3H), 2.06-1.74 (m, 3H), 1.72-1.59 (m, 1H), 1.45 (s, 9H), 1.06-0.96 (m, 2H), 0.74-0.63 (m, 2H).

LC/MS (Method 1, ESIneg): Rₜ=1.87 min, m/z=447.17 [M−H]⁻.

### Example 168A

### tert-Butyl 2-({3-cyclopropyl-5-methyl-2,4-dioxo-1-[(2S)-tetrahydrofuran-2-ylmethyl]-1,2,3,4-tetrahydrothieno[2,3-d]pyrimidin-6-yl}methylene)hydrazinecarboxylate

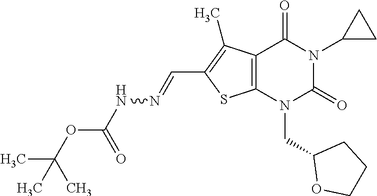 [SMILES: Cc1c(C=NNC(=O)OC(C)(C)C)sc2c1c(=O)n(C1CC1)c(=O)n2C[C@@H]1CCCO1]

Analogously to the method described in Ex. 156A, 200 mg (0.598 mmol) of the compound from Ex. 61A and 119 mg (0.897 mmol) of tert-butyl hydrazinecarboxylate were used to prepare 241 mg (89% of theory) of the title compound.

¹H-NMR (400 MHz, DMSO-d₆, δ/ppm): 10.83 (br. s, 1H), 8.28 (s, 1H), 4.26-4.15 (m, 1H), 4.06 (dd, 1H), 3.82-3.67 (m, 2H), 3.66-3.57 (m, 1H), 2.65-2.56 (m, 1H), 2.43 (s, 3H), 2.06-1.74 (m, 3H), 1.71-1.58 (m, 1H), 1.45 (s, 9H), 1.06-0.94 (m, 2H), 0.74-0.64 (m, 2H).

LC/MS (Method 1, ESIneg): Rₜ=1.87 min, m/z=447.17 [M−H]⁻.

### Example 169A

### tert-Butyl 2-{[1,5-dimethyl-3-(1-methylcyclopropyl)-2,4-dioxo-1,2,3,4-tetrahydrothieno[2,3-d]pyrimidin-6-yl]methylene}hydrazinecarboxylate

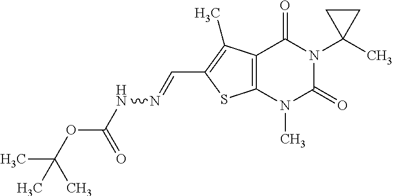 [SMILES: Cc1c(C=NNC(=O)OC(C)(C)C)sc2c1c(=O)n(C1(C)CC1)c(=O)n2C]

Analogously to the method described in Ex. 156A, 257 mg (0.923 mmol) of the compound from Ex. 62A and 183 mg (1.39 mmol) of tert-butyl hydrazinecarboxylate were used to prepare 362 mg (99% of theory) of the title compound.

¹H-NMR (400 MHz, DMSO-d₆, δ/ppm): 10.82 (br. s, 1H), 8.28 (s, 1H), 3.42 (s, 3H), 2.45 (s, 3H), 1.45 (s, 9H), 1.33 (s, 3H), 0.97-0.76 (m, 4H).

LC/MS (Method 2, ESIneg): Rₜ=0.96 min, m/z=391 [M−H]⁻.

### Example 170A

### tert-Butyl 2-{[1-butyl-5-methyl-3-(1-methylcyclopropyl)-2,4-dioxo-1,2,3,4-tetrahydrothieno[2,3-d]pyrimidin-6-yl]methylene}hydrazinecarboxylate

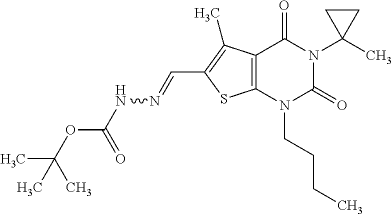 [SMILES: CCCCn1c(=O)n(C2(C)CC2)c(=O)c2c(C)c(C=NNC(=O)OC(C)(C)C)sc21]

Analogously to the method described in Ex. 156A, 300 mg (0.936 mmol) of the compound from Ex. 63A and 185 mg (1.40 mmol) of tert-butyl hydrazinecarboxylate were used to prepare 397 mg (97% of theory) of the title compound.

¹H-NMR (400 MHz, DMSO-d₆, δ/ppm): 10.84 (br. s, 1H), 8.28 (s, 1H), 4.01-3.71 (m, 2H), 2.44 (s, 3H), 1.66 (quin, 2H), 1.45 (s, 9H), 1.41-1.28 (m, 2H), 1.33 (s, 3H), 0.98-0.75 (m, 4H), 0.92 (t, 3H).

LC/MS (Method 1, ESIneg): Rₜ=2.26 min, m/z=433.19 [M−H]⁻.

### Example 171A

### tert-Butyl 2-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methylene}hydrazinecarboxylate

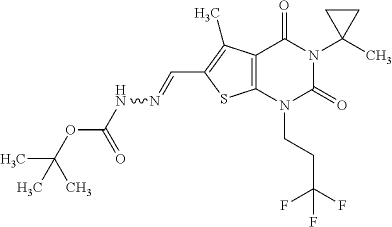 [SMILES: Cc1c(C=NNC(=O)OC(C)(C)C)sc2c1c(=O)n(C1(C)CC1)c(=O)n2CCC(F)(F)F]

To a solution of 1.07 g (2.80 mmol, 94% purity) of the compound from Ex. 64A in 34 ml of ethanol were added first 555 mg (4.20 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with water and neutralized by adding saturated aqueous sodium hydrogencarbonate solution. The precipitated solids were filtered off with suction, washed with a little water and dried under high vacuum. 1.34 g (94% of theory, 94% purity) of the title compound were obtained, which was used for subsequent reactions without further purification.

¹H-NMR (400 MHz, DMSO-d₆, δ/ppm): 10.86 (br. s, 1H), 8.29 (s, 1H), 4.28-4.09 (m, 2H), 2.81-2.73 (m, 2H), 2.45 (s, 3H), 1.45 (s, 9H), 1.34 (s, 3H), 0.95-0.80 (m, 4H).

LC/MS (Method 2, ESIpos): Rₜ=1.11 min, m/z=475 [M+H]⁺.

### Example 172A

### tert-Butyl 2-{[1-(cyclobutylmethyl)-5-methyl-3-(1-methylcyclopropyl)-2,4-dioxo-1,2,3,4-tetrahydrothieno[2,3-d]pyrimidin-6-yl]methylene}hydrazinecarboxylate

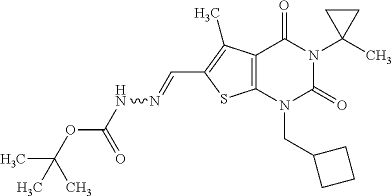 [SMILES: Cc1c(C=NNC(=O)OC(C)(C)C)sc2c1c(=O)n(C1(C)CC1)c(=O)n2CC1CCC1]

Analogously to the method described in Ex. 156A, 290 mg (0.872 mmol) of the compound from Ex. 65A and 173 mg (1.31 mmol) of tert-butyl hydrazinecarboxylate were used to prepare 362 mg (92% of theory) of the title compound.

¹H-NMR (400 MHz, DMSO-d₆, δ/ppm): 10.84 (br. s, 1H), 8.27 (s, 1H), 4.12-3.76 (m, 2H), 2.83-2.70 (m, 1H), 2.43 (s, 3H), 2.06-1.91 (m, 2H), 1.89-1.75 (m, 4H), 1.45 (s, 9H), 1.33 (s, 3H), 0.97-0.72 (m, 4H).

LC/MS (Method 2, ESIneg): Rₜ=1.20 min, m/z=445 [M−H]⁻.

### Example 173A

### tert-Butyl 2-({1-[(2,2-difluorocyclopropyl)methyl]-5-methyl-3-(1-methylcyclopropyl)-2,4-dioxo-1,2,3,4-tetrahydrothieno[2,3-d]pyrimidin-6-yl}methylene)hydrazinecarboxylate (racemate)

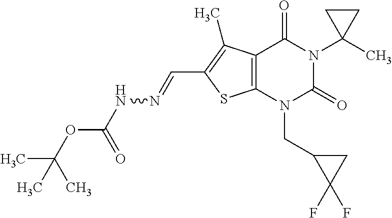 [SMILES: Cc1c(C=NNC(=O)OC(C)(C)C)sc2c1c(=O)n(C1(C)CC1)c(=O)n2CC1CC1(F)F]

Analogously to the method described in Ex. 156A, 660 mg (1.86 mmol) of the compound from Ex. 66A and 369 mg (2.79 mmol) of tert-butyl hydrazinecarboxylate were used to prepare 820 mg (91% of theory, 97% purity) of the title compound.

¹H-NMR (400 MHz, DMSO-d₆, δ/ppm): 10.85 (br. s, 1H), 8.29 (s, 1H), 4.28-3.81 (m, 2H), 2.45 (s, 3H), 2.30-2.14 (m, 1H), 1.79-1.64 (m, 1H), 1.52-1.41 (m, 1H), 1.45 (s, 9H), 1.34 (s, 3H), 0.98-0.77 (m, 4H).

LC/MS (Method 1, ESIneg): Rₜ=2.09 min, m/z=467.16 [M−H]⁻.

### Example 174A

### tert-Butyl 2-{[1-(cyanomethyl)-5-methyl-3-(1-methylcyclopropyl)-2,4-dioxo-1,2,3,4-tetrahydrothieno[2,3-d]pyrimidin-6-yl]methylene}hydrazinecarboxylate

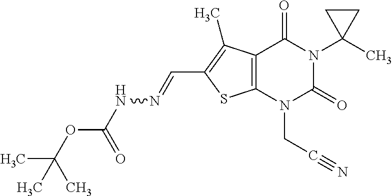 [SMILES: Cc1c(C=NNC(=O)OC(C)(C)C)sc2c1c(=O)n(C1(C)CC1)c(=O)n2CC#N]

Analogously to the method described in Ex. 156A, 250 mg (0.824 mmol) of the compound from Ex. 67A and 163 mg (1.24 mmol) of tert-butyl hydrazinecarboxylate were used to prepare 290 mg (75% of theory, 90% purity) of the title compound.

¹H-NMR (400 MHz, DMSO-d₆, δ/ppm): 10.89 (br. s, 1H), 8.30 (s, 1H), 5.14 (br. d, 2H), 2.45 (s, 3H), 1.46 (s, 9H), 1.34 (s, 3H), 0.92-0.85 (m, 4H).

LC/MS (Method 1, ESIneg): Rₜ=1.81 min, m/z=416.14 [M−H]⁻.

### Example 175A

### tert-Butyl 2-{[1-(2-methoxyethyl)-5-methyl-3-(1-methylcyclopropyl)-2,4-dioxo-1,2,3,4-tetrahydrothieno[2,3-d]pyrimidin-6-yl]methylene}hydrazinecarboxylate

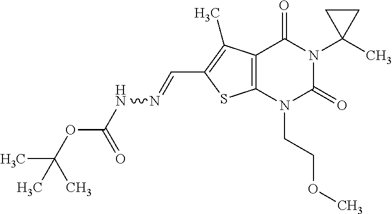 [SMILES: COCCn1c(=O)n(C2(C)CC2)c(=O)c2c(C)c(C=NNC(=O)OC(C)(C)C)sc21]

To a solution of 932 mg (2.10 mmol, 72% purity) of the compound from Ex. 68A in 25 ml of ethanol were added first 412 mg (3.12 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with water and neutralized by adding saturated aqueous sodium hydrogencarbonate solution. The precipitated solids were filtered off with suction, washed with a little water and dried under high vacuum. 1.17 g (95% of theory, 74% purity) of the title compound were obtained, which was used for subsequent reactions without further purification.

LC/MS (Method 2, ESIpos): Rₜ=0.99 min, m/z=437 [M+H]⁺.

### Example 176A

### tert-Butyl 2-({5-methyl-3-(1-methylcyclopropyl)-2,4-dioxo-1-[2-(trifluoromethoxy)ethyl]-1,2,3,4-tetrahydrothieno[2,3-d]pyrimidin-6-yl}methylene)hydrazinecarboxylate

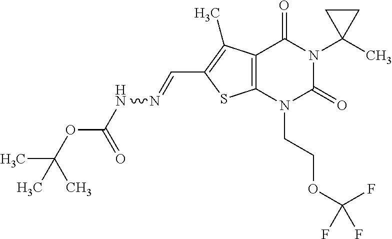 [SMILES: Cc1c(C=NNC(=O)OC(C)(C)C)sc2c1c(=O)n(C1(C)CC1)c(=O)n2CCOC(F)(F)F]

To a solution of 360 mg (0.957 mmol) of the compound from Ex. 69A in 10 ml of ethanol were added first 190 mg (1.44 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 432 mg (73% of theory, 80% purity) of the title compound were obtained.

¹H-NMR (400 MHz, DMSO-d₆, δ/ppm): 10.84 (br. s, 1H), 8.28 (s, 1H), 4.43-4.38 (m, 2H), 4.34-4.07 (m, 2H), 2.44 (s, 3H), 1.45 (s, 9H), 1.34 (s, 3H), 0.98-0.78 (m, 4H).

LC/MS (Method 1, ESIneg): Rₜ=2.11 min, m/z=489.14 [M−H]⁻.

### Example 177A

### tert-Butyl 2-{[1-(2-ethoxyethyl)-5-methyl-3-(1-methylcyclopropyl)-2,4-dioxo-1,2,3,4-tetrahydrothieno[2,3-d]pyrimidin-6-yl]methylene}hydrazinecarboxylate

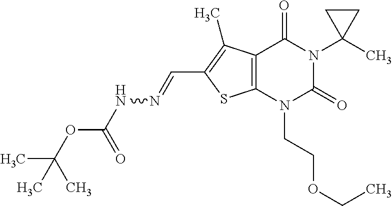 [SMILES: CCOCCn1c(=O)n(C2(C)CC2)c(=O)c2c(C)c(C=NNC(=O)OC(C)(C)C)sc21]

Analogously to the method described in Ex. 156A, 250 mg (0.743 mmol) of the compound from Ex. 70A and 147 mg (1.11 mmol) of tert-butyl hydrazinecarboxylate were used to prepare 316 mg (66% of theory, 70% purity) of the title compound. In this case, the product was additionally also purified by means of MPLC (Biotage Isolera One, SNAP KP-Sil cartridge, 50 g of silica gel, eluent: cyclohexane/ethyl acetate 2:1).

¹H-NMR (400 MHz, DMSO-d₆, δ/ppm): 10.83 (br. s, 1H), 8.28 (s, 1H), 4.22-3.87 (m, 2H), 3.70-3.61 (m, 2H), 3.45 (q, 2H), 2.44 (s, 3H), 1.45 (s, 9H), 1.34 (s, 3H), 1.05 (t, 3H), 0.96-0.78 (m, 4H).

LC/MS (Method 1, ESIneg): Rₜ=2.00 min, m/z=449.19 [M−H]⁻.

### Example 178A

### tert-Butyl 2-{[1-(2-isopropoxyethyl)-5-methyl-3-(1-methylcyclopropyl)-2,4-dioxo-1,2,3,4-tetrahydrothieno[2,3-d]pyrimidin-6-yl]methylene}hydrazinecarboxylate

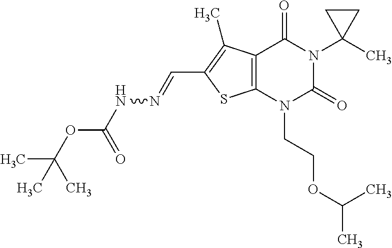 [SMILES: Cc1c(C=NNC(=O)OC(C)(C)C)sc2c1c(=O)n(C1(C)CC1)c(=O)n2CCOC(C)C]

Analogously to the method described in Ex. 156A, 280 mg (0.799 mmol) of the compound from Ex. 71A and 158 mg (1.20 mmol) of tert-butyl hydrazinecarboxylate were used to prepare 370 mg (99% of theory) of the title compound. The product here was additionally purified by means of MPLC (Biotage Isolera One, SNAP KP-Sil cartridge, 50 g of silica gel, eluent: cyclohexane/ethyl acetate 2:1).

¹H-NMR (400 MHz, DMSO-d₆, δ/ppm): 10.83 (br. s, 1H), 8.28 (s, 1H), 4.14-3.85 (m, 2H), 3.66 (t, 2H), 3.56 (sept, 1H), 2.44 (s, 3H), 1.45 (s, 9H), 1.33 (s, 3H), 1.01 (d, 6H), 0.96-0.75 (m, 4H).

LC/MS (Method 1, ESIneg): Rₜ=2.13 min, m/z=463.20 [M−H]⁻.

### Example 179A

### tert-Butyl 2-({5-methyl-3-(1-methylcyclopropyl)-2,4-dioxo-1-[(2R)-tetrahydrofuran-2-ylmethyl]-1,2,3,4-tetrahydrothieno[2,3-d]pyrimidin-6-yl}methylene)hydrazinecarboxylate

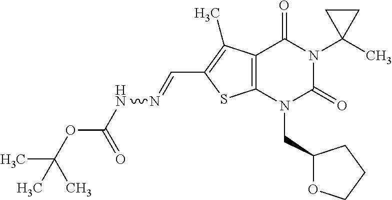 [SMILES: Cc1c(C=NNC(=O)OC(C)(C)C)sc2c1c(=O)n(C1(C)CC1)c(=O)n2C[C@H]1CCCO1]

Analogously to the method described in Ex. 156A, 200 mg (0.574 mmol) of the compound from Ex. 72A and 114 mg (0.861 mmol) of tert-butyl hydrazinecarboxylate were used to prepare 240 mg (90% of theory) of the title compound.

¹H-NMR (400 MHz, DMSO-d₆, δ/ppm): 10.83 (br. s, 1H), 8.28 (s, 1H), 4.34-3.92 (m, 2H), 3.89-3.53 (m, 3H), 2.44 (s, 3H), 2.07-1.75 (m, 3H), 1.73-1.59 (m, 1H), 1.45 (s, 9H), 1.34 (s, 3H), 0.96-0.78 (m, 4H).

LC/MS (Method 1, ESIneg): Rₜ=2.00 min, m/z=461.19 [M−H]⁻.

### Example 180A

### tert-Butyl 2-({5-methyl-3-(1-methylcyclopropyl)-2,4-dioxo-1-[(2S)-tetrahydrofuran-2-ylmethyl]-1,2,3,4-tetrahydrothieno[2,3-d]pyrimidin-6-yl}methylene)hydrazinecarboxylate

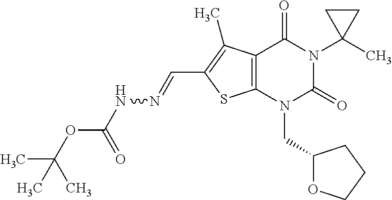 [SMILES: Cc1c(C=NNC(=O)OC(C)(C)C)sc2c1c(=O)n(C1(C)CC1)c(=O)n2C[C@@H]1CCCO1]

Analogously to the method described in Ex. 156A, 275 mg (0.789 mmol) of the compound from Ex. 73A and 156 mg (1.18 mmol) of tert-butyl hydrazinecarboxylate were used to prepare 342 mg (93% of theory) of the title compound.

¹H-NMR (400 MHz, DMSO-d₆, δ/ppm): 10.83 (br. s, 1H), 8.28 (s, 1H), 4.33-3.92 (m, 2H), 3.87-3.54 (m, 3H), 2.44 (s, 3H), 2.06-1.74 (m, 3H), 1.73-1.58 (m, 1H), 1.45 (s, 9H), 1.34 (s, 3H), 0.99-0.72 (m, 4H).

LC/MS (Method 1, ESIneg): Rₜ=2.00 min, m/z=461.19 [M−H]⁻.

### Example 181A

### tert-Butyl 2-({1-(2-methoxyethyl)-5-methyl-2,4-dioxo-3-[1-(trifluoromethyl)cyclopropyl]-1,2,3,4-tetrahydrothieno[2,3-d]pyrimidin-6-yl}methylene)hydrazinecarboxylate

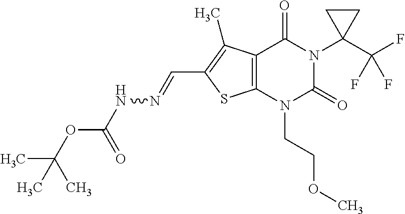 [SMILES: COCCn1c(=O)n(C2(C(F)(F)F)CC2)c(=O)c2c(C)c(C=NNC(=O)OC(C)(C)C)sc21]

Analogously to the method described in Ex. 156A, 365 mg (0.970 mmol) of the compound from Ex. 74A and 192 mg (1.46 mmol) of tert-butyl hydrazinecarboxylate were used to prepare 450 mg (94% of theory) of the title compound.

¹H-NMR (400 MHz, DMSO-d₆, δ/ppm): 10.87 (br. s, 1H), 8.28 (s, 1H), 4.15-3.96 (m, 2H), 3.64 (t, 2H), 3.26 (s, 3H), 2.43 (s, 3H), 1.66-1.51 (m, 2H), 1.45 (s, 9H), 1.39-1.32 (m, 2H).

LC/MS (Method 1, ESIneg): Rₜ=1.98 min, m/z=489.14 [M−H]⁻.

### Example 182A

### tert-Butyl 2-{[3-(1-ethylcyclopropyl)-5-methyl-2,4-dioxo-1-(3,3,3-trifluoropropyl)-1,2,3,4-tetrahydrothieno[2,3-d]pyrimidin-6-yl]methylene}hydrazinecarboxylate

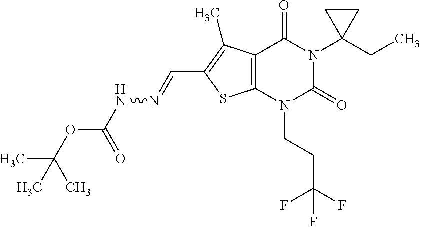 [SMILES: CCC1(n2c(=O)c3c(C)c(C=NNC(=O)OC(C)(C)C)sc3n(CCC(F)(F)F)c2=O)CC1]

Analogously to the method described in Ex. 156A, 166 mg (0.443 mmol) of the compound from Ex. 81A and 88 mg (0.665 mmol) of tert-butyl hydrazinecarboxylate were used to prepare 201 mg (74% of theory, 80% purity) of the title compound, which was used for subsequent reactions without further purification.

¹H-NMR (400 MHz, DMSO-d₆, δ/ppm): 10.86 (br. s, 1H), 8.29 (s, 1H), 4.11 (q, 2H), 2.85-2.69 (m, 2H), 2.44 (s, 3H), 1.69 (q, 2H), 1.45 (s, 9H), 1.01-0.88 (m, 2H), 0.87-0.78 (m, 2H), 0.82 (t, 3H).

LC/MS (Method 1, ESIneg): Rₜ=2.23 min, m/z=487.16 [M−H]⁻.

### Example 183A

### tert-Butyl 2-{[3-(1-ethylcyclopropyl)-1-(2-methoxyethyl)-5-methyl-2,4-dioxo-1,2,3,4-tetrahydrothieno[2,3-d]pyrimidin-6-yl]methylene}hydrazinecarboxylate

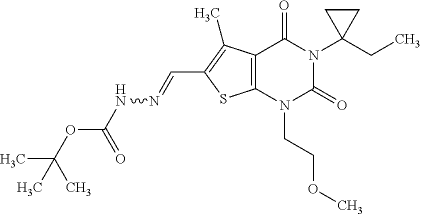 [SMILES: CCC1(n2c(=O)c3c(C)c(C=NNC(=O)OC(C)(C)C)sc3n(CCOC)c2=O)CC1]

Analogously to the method described in Ex. 156A, 212 mg (0.616 mmol) of the compound from Ex. 82A and 122 mg (0.924 mmol) of tert-butyl hydrazinecarboxylate were used to prepare 274 mg (86% of theory, 88% purity) of the title compound, which was used for subsequent reactions without further purification.

¹H-NMR (400 MHz, DMSO-d₆, δ/ppm): 10.83 (br. s, 1H), 8.27 (s, 1H), 4.12-3.93 (m, 2H), 3.63 (t, 2H), 3.25 (s, 3H), 2.43 (s, 3H), 1.70 (q, 2H), 1.45 (s, 9H), 1.01-0.76 (m, 4H), 0.82 (t, 3H).

LC/MS (Method 1, ESIneg): Rₜ=2.05 min, m/z=449.19 [M−H]⁻.

### Example 184A

### tert-Butyl 2-{[3-cyclobutyl-5-methyl-2,4-dioxo-1-(3,3,3-trifluoropropyl)-1,2,3,4-tetrahydrothieno[2,3-d]pyrimidin-6-yl]methylene}hydrazinecarboxylate

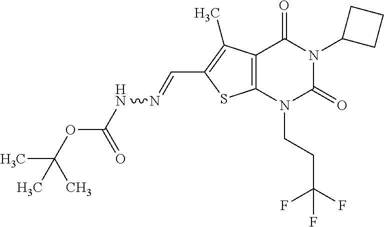 [SMILES: Cc1c(C=NNC(=O)OC(C)(C)C)sc2c1c(=O)n(C1CCC1)c(=O)n2CCC(F)(F)F]

To a solution of 357 mg (0.991 mmol) of the compound from Ex. 83A in 9 ml of ethanol were added first 196 mg (1.49 mmol) of tert-butyl hydrazinecarboxylate and then 2 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 451 mg (92% of theory, 96% purity) of the title compound were obtained.

¹H-NMR (400 MHz, DMSO-d₆, δ/ppm): 10.87 (br. s, 1H), 8.29 (s, 1H), 5.19 (quin, 1H), 4.11 (br. t, 2H), 2.90-2.69 (m, 4H), 2.44 (s, 3H), 2.17 (q, 2H), 1.88-1.65 (m, 2H), 1.45 (s, 9H).

LC/MS (Method 1, ESIneg): Rₜ=2.31 min, m/z=473.15 [M−H]⁻.

### Example 185A

### tert-Butyl 2-{[3-cyclobutyl-1-(2-methoxyethyl)-5-methyl-2,4-dioxo-1,2,3,4-tetrahydrothieno[2,3-d]pyrimidin-6-yl]methylene}hydrazinecarboxylate

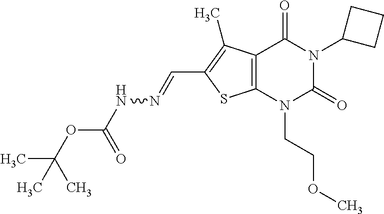 [SMILES: COCCn1c(=O)n(C2CCC2)c(=O)c2c(C)c(C=NNC(=O)OC(C)(C)C)sc21]

To a solution of 280 mg (0.869 mmol) of the compound from Ex. 84A in 9 ml of ethanol were added first 172 mg (1.30 mmol) of tert-butyl hydrazinecarboxylate and then 2 drops of concentrated hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 354 mg (91% of theory) of the title compound were obtained.

¹H-NMR (400 MHz, DMSO-d₆, δ/ppm): 10.84 (br. s, 1H), 8.28 (s, 1H), 5.20 (quin, 1H), 4.04 (t, 2H), 3.64 (t, 2H), 3.25 (s, 3H), 2.91-2.76 (m, 2H), 2.43 (s, 3H), 2.17 (dtd, 2H), 1.88-1.65 (m, 2H), 1.45 (s, 9H).

LC/MS (Method 1, ESIneg): Rₜ=2.12 min, m/z=435.17 [M−H]⁻.

### Example 186A

### tert-Butyl 2-{[3-(3,3-difluorocyclobutyl)-5-methyl-2,4-dioxo-1-(3,3,3-trifluoropropyl)-1,2,3,4-tetrahydrothieno[2,3-d]pyrimidin-6-yl]methylene}hydrazinecarboxylate

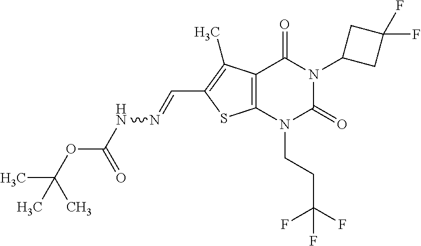 [SMILES: Cc1c(C=NNC(=O)OC(C)(C)C)sc2c1c(=O)n(C1CC(F)(F)C1)c(=O)n2CCC(F)(F)F]

To a solution of 519 mg (1.26 mmol) of the compound from Ex. 85A in 15 ml of ethanol were added first 250 mg (1.89 mmol) of tert-butyl hydrazinecarboxylate and then 5 drops of concentrated hydrochloric acid. After the reaction mixture had been stirred at RT for about 18 h, the majority of the ethanol was removed on a rotary evaporator. The remaining residue was diluted with water and neutralized by adding saturated aqueous sodium hydrogencarbonate solution. The precipitated solids were filtered off with suction, washed with water and dried under high vacuum. 670 mg (97% of theory, 93% purity) of the title compound were obtained, which was used for subsequent reactions without further purification.

LC/MS (Method 2, ESIpos): Rₜ=1.17 min, m/z=511 [M+H]⁺.

### Example 187A

### tert-Butyl 2-{[3-(3,3-difluorocyclobutyl)-1-(2-methoxyethyl)-5-methyl-2,4-dioxo-1,2,3,4-tetrahydrothieno[2,3-d]pyrimidin-6-yl]methylene}hydrazinecarboxylate

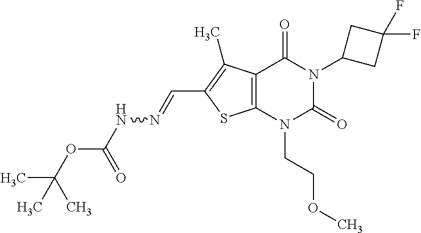 [SMILES: COCCn1c(=O)n(C2CC(F)(F)C2)c(=O)c2c(C)c(C=NNC(=O)OC(C)(C)C)sc21]

To a solution of 574 mg (1.40 mmol, 87% purity) of the compound from Ex. 86A in 17 ml of ethanol were added first 277 mg (2.10 mmol) of tert-butyl hydrazinecarboxylate and then 5 drops of concentrated hydrochloric acid. After the reaction mixture had been stirred at RT for about 18 h, the majority of the ethanol was removed on a rotary evaporator. The remaining residue was diluted with water and neutralized by adding saturated aqueous sodium hydrogencarbonate solution. The precipitated solids were filtered off with suction, washed with a little water and dried under high vacuum. 697 mg (91% of theory, 86% purity) of the title compound were obtained, which was used for subsequent reactions without further purification.

LC/MS (Method 2, ESIpos): Rₜ=1.10 min, m/z=473 [M+H]⁺.

### Example 188A

### tert-Butyl 2-{[5-methyl-3-(1-methylcyclobutyl)-2,4-dioxo-1-(3,3,3-trifluoropropyl)-1,2,3,4-tetrahydrothieno[2,3-d]pyrimidin-6-yl]methylene}hydrazinecarboxylate

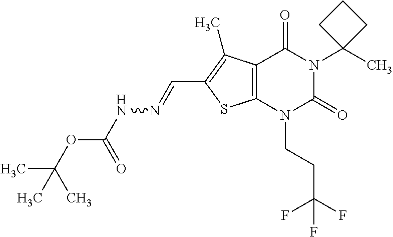 [SMILES: Cc1c(C=NNC(=O)OC(C)(C)C)sc2c1c(=O)n(C1(C)CCC1)c(=O)n2CCC(F)(F)F]

Analogously to the method described in Ex. 156A, 300 mg (0.761 mmol, 95% purity) of the compound from Ex. 88A and 151 mg (1.14 mmol) of tert-butyl hydrazinecarboxylate were used to prepare 346 mg (93% of theory) of the title compound.

¹H-NMR (400 MHz, DMSO-d₆, δ/ppm): 10.86 (br. s, 1H), 8.29 (s, 1H), 4.09 (t, 2H), 2.84-2.69 (m, 2H), 2.42 (s, 3H), 2.36-2.22 (m, 4H), 1.82-1.57 (m, 2H), 1.52 (s, 3H), 1.45 (s, 9H).

LC/MS (Method 1, ESIneg): Rₜ=2.32 min, m/z=487.16 [M−H]⁻.

### Example 189A

### tert-Butyl 2-{[1-(2-methoxyethyl)-5-methyl-3-(1-methylcyclobutyl)-2,4-dioxo-1,2,3,4-tetrahydrothieno[2,3-d]pyrimidin-6-yl]methylene}hydrazinecarboxylate

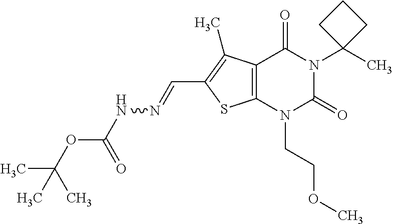 [SMILES: COCCn1c(=O)n(C2(C)CCC2)c(=O)c2c(C)c(C=NNC(=O)OC(C)(C)C)sc21]

Analogously to the method described in Ex. 156A, 300 mg (0.892 mmol) of the compound from Ex. 89A and 177 mg (1.34 mmol) of tert-butyl hydrazinecarboxylate were used to prepare 400 mg (99% of theory) of the title compound.

¹H-NMR (400 MHz, DMSO-d₆, δ/ppm): 10.83 (br. s, 1H), 8.27 (s, 1H), 4.06-3.96 (m, 2H), 3.62 (t, 2H), 3.26 (s, 3H), 2.41 (s, 3H), 2.37-2.22 (m, 4H), 1.80-1.57 (m, 2H), 1.52 (s, 3H), 1.45 (s, 9H).

LC/MS (Method 1, ESIneg): Rₜ=2.13 min, m/z=449.19 [M−H]⁻.

### Example 190A

### tert-Butyl 2-{[3-(trans-3-methoxycyclobutyl)-5-methyl-2,4-dioxo-1-(3,3,3-trifluoropropyl)-1,2,3,4-tetrahydrothieno[2,3-d]pyrimidin-6-yl]methylene}hydrazinecarboxylate

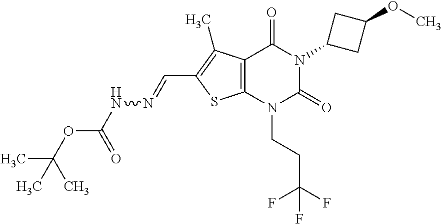 [SMILES: CO[C@H]1C[C@H](n2c(=O)c3c(C)c(C=NNC(=O)OC(C)(C)C)sc3n(CCC(F)(F)F)c2=O)C1]

To a solution of 778 mg (1.80 mmol, 91% purity) of the compound from Ex. 90A in 15 ml of ethanol were added first 358 mg (2.71 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was admixed with water and neutralized with saturated aqueous sodium hydrogencarbonate solution. The precipitated solids were filtered off, washed with water and dried under high vacuum. 0.95 g (86% of theory, 82% purity) of the title compound was obtained, which was used for subsequent reactions without further purification.

LC/MS (Method 2, ESIpos): Rₜ=1.13 min, m/z=505 [M+H]⁺.

### Example 191A

### tert-Butyl 2-{[3-(trans-3-methoxycyclobutyl)-1-(2-methoxyethyl)-5-methyl-2,4-dioxo-1,2,3,4-tetrahydrothieno[2,3-d]pyrimidin-6-yl]methylene}hydrazinecarboxylate

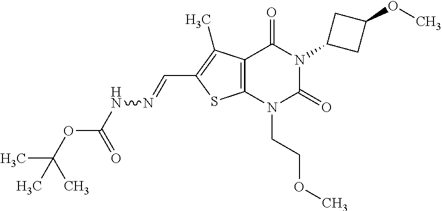 [SMILES: COCCn1c(=O)n([C@H]2C[C@H](OC)C2)c(=O)c2c(C)c(C=NNC(=O)OC(C)(C)C)sc21]

To a solution of 815 mg (1.89 mmol, 81% purity) of the compound from Ex. 91A in 22 ml of ethanol were added first 373 mg (2.82 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The residue that remained was admixed with water and ethyl acetate. The organic phase was washed with saturated aqueous sodium hydrogencarbonate solution, water and saturated aqueous sodium chloride solution. After drying over anhydrous magnesium sulfate, the mixture was filtered and concentrated to dryness. 1.08 g (88% of theory, 71% purity) of the title compound were obtained, which was used for subsequent reactions without further purification.

LC/MS (Method 2, ESIpos): Rₜ=1.02 min, m/z=467 [M+H]⁺.

### Example 192A

### tert-Butyl 2-{[3-(cis-3-methoxycyclobutyl)-5-methyl-2,4-dioxo-1-(3,3,3-trifluoropropyl)-1,2,3,4-tetrahydrothieno[2,3-d]pyrimidin-6-yl]methylene}hydrazinecarboxylate

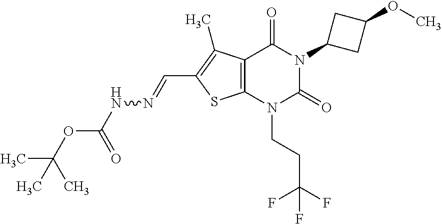 [SMILES: CO[C@H]1C[C@@H](n2c(=O)c3c(C)c(C=NNC(=O)OC(C)(C)C)sc3n(CCC(F)(F)F)c2=O)C1]

To a solution of 500 mg (1.23 mmol) of the compound from Ex. 92A in 15 ml of ethanol were added first 244 mg (1.84 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with water and ethyl acetate, and neutralized by adding saturated aqueous sodium hydrogencarbonate solution. The organic phase was washed with water and saturated sodium chloride solution, dried over anhydrous magnesium sulfate, filtered and concentrated. The crude product obtained, after drying under high vacuum, gave 0.53 g (81% of theory, 95% purity) of the title compound, which was used for subsequent reactions without further purification.

LC/MS (Method 2, ESIpos): Rₜ=1.11 min, m/z=505 [M+H]⁺.

### Example 193A

### tert-Butyl 2-{[3-(cis-3-methoxycyclobutyl)-1-(2-methoxyethyl)-5-methyl-2,4-dioxo-1,2,3,4-tetrahydrothieno[2,3-d]pyrimidin-6-yl]methylene}hydrazinecarboxylate

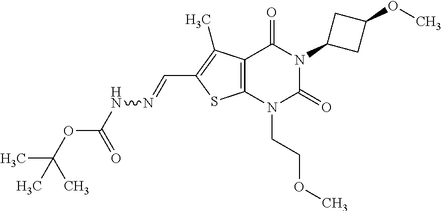 [SMILES: COCCn1c(=O)n([C@H]2C[C@@H](OC)C2)c(=O)c2c(C)c(C=NNC(=O)OC(C)(C)C)sc21]

To a solution of 500 mg (1.35 mmol, 87% purity) of the compound from Ex. 93A in 16 ml of ethanol were added first 267 mg (2.02 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The remaining residue was diluted with water and ethyl acetate, and neutralized by adding saturated aqueous sodium hydrogencarbonate solution. The organic phase was washed with water and saturated sodium chloride solution, dried over anhydrous magnesium sulfate, filtered and concentrated. The crude product obtained, after drying under high vacuum, gave 0.84 g (100% of theory, 80% purity) of the title compound, which was used for subsequent reactions without further purification.

LC/MS (Method 2, ESIpos): Rₜ=1.00 min, m/z=467 [M+H]⁺.

### Example 194A

### tert-Butyl 2-{[3-(3,3-dimethylcyclobutyl)-5-methyl-2,4-dioxo-1-(3,3,3-trifluoropropyl)-1,2,3,4-tetrahydrothieno[2,3-d]pyrimidin-6-yl]methylene}hydrazinecarboxylate

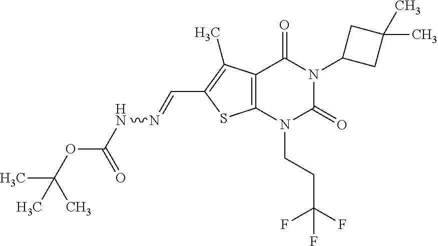 [SMILES: Cc1c(C=NNC(=O)OC(C)(C)C)sc2c1c(=O)n(C1CC(C)(C)C1)c(=O)n2CCC(F)(F)F]

To a solution of 500 mg (1.18 mmol, 95% purity) of the compound from Ex. 94A in 14 ml of ethanol were added first 235 mg (1.78 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The residue that remained was admixed with water and ethyl acetate. The organic phase was washed with saturated aqueous sodium hydrogencarbonate solution, water and saturated aqueous sodium chloride solution. After drying over anhydrous magnesium sulfate, the mixture was filtered and concentrated to dryness. 0.42 g (71% of theory, 87% purity) of the title compound was obtained, which was used for subsequent reactions without further purification.

LC/MS (Method 2, ESIpos): Rₜ=1.34 min, m/z=503 [M+H]⁺.

### Example 195A

### tert-Butyl 2-{[3-(3,3-dimethylcyclobutyl)-1-(2-methoxyethyl)-5-methyl-2,4-dioxo-1,2,3,4-tetrahydrothieno[2,3-d]pyrimidin-6-yl]methylene}hydrazinecarboxylate

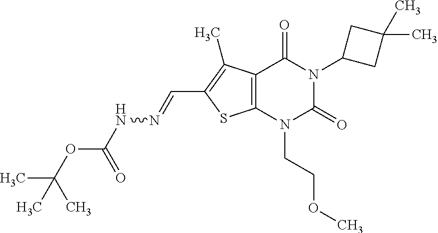 [SMILES: COCCn1c(=O)n(C2CC(C)(C)C2)c(=O)c2c(C)c(C=NNC(=O)OC(C)(C)C)sc21]

To a solution of 500 mg (1.43 mmol, 91% purity) of the compound from Ex. 95A in 17 ml of ethanol were added first 283 mg (2.14 mmol) of tert-butyl hydrazinecarboxylate and then 3 drops of concentrated hydrochloric acid. After the reaction mixture had been stirred at RT for about 18 h, the majority of the ethanol was removed on a rotary evaporator. The residue that remained was admixed with water and ethyl acetate. The organic phase was washed with saturated aqueous sodium hydrogencarbonate solution, water and saturated aqueous sodium chloride solution. After drying over anhydrous magnesium sulfate, the mixture was filtered and concentrated to dryness. 0.74 g (64% of theory, 81% purity) of the title compound were obtained, which was used for subsequent reactions without further purification.

LC/MS (Method 2, ESIpos): Rₜ=1.25 min, m/z=467 [M+H]⁺.

### Example 196A

### tert-Butyl 2-{[5-methyl-2,4-dioxo-3-(spiro[3.3]hept-2-yl)-1-(3,3,3-trifluoropropyl)-1,2,3,4-tetrahydrothieno[2,3-d]pyrimidin-6-yl]methylene}hydrazinecarboxylate

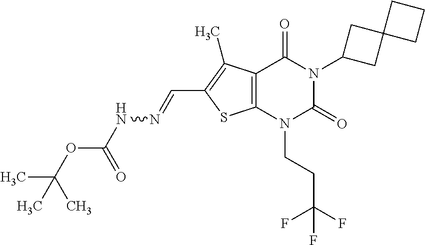 [SMILES: Cc1c(C=NNC(=O)OC(C)(C)C)sc2c1c(=O)n(C1CC3(CCC3)C1)c(=O)n2CCC(F)(F)F]

Analogously to the method described in Ex. 156A, 400 mg (0.999 mmol) of the compound from Ex. 96A and 198 mg (1.50 mmol) of tert-butyl hydrazinecarboxylate were used to prepare 498 mg (96% of theory) of the title compound.

¹H-NMR (400 MHz, DMSO-d₆, δ/ppm): 10.86 (br. s, 1H), 8.29 (s, 1H), 5.03 (quin, 1H), 4.10 (t, 2H), 2.89-2.69 (m, 4H), 2.43 (s, 3H), 2.31-2.19 (m, 2H), 2.11-2.03 (m, 2H), 2.01-1.94 (m, 2H), 1.87-1.74 (m, 2H), 1.45 (s, 9H).

LC/MS (Method 1, ESIneg): Rₜ=2.58 min, m/z=513.18 [M−H]⁻.

### Example 197A

### tert-Butyl 2-{[1-(2-methoxyethyl)-5-methyl-2,4-dioxo-3-(spiro[3.3]hept-2-yl)-1,2,3,4-tetrahydrothieno[2,3-d]pyrimidin-6-yl]methylene}hydrazinecarboxylate

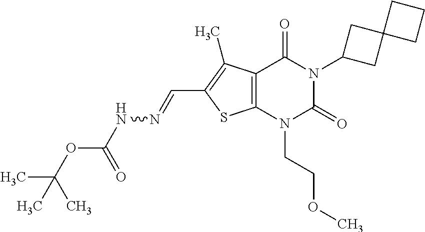 [SMILES: COCCn1c(=O)n(C2CC3(CCC3)C2)c(=O)c2c(C)c(C=NNC(=O)OC(C)(C)C)sc21]

Analogously to the method described in Ex. 156A, 400 mg (1.10 mmol) of the compound from Ex. 97A and 219 mg (1.66 mmol) of tert-butyl hydrazinecarboxylate were used to prepare 510 mg (96% of theory) of the title compound.

¹H-NMR (400 MHz, DMSO-d₆, δ/ppm): 10.84 (br. s, 1H), 8.27 (s, 1H), 5.04 (quin, 1H), 4.03 (t, 2H), 3.63 (t, 2H), 3.25 (s, 3H), 2.90-2.77 (m, 2H), 2.42 (s, 3H), 2.24 (td, 2H), 2.06 (t, 2H), 2.01-1.93 (m, 2H), 1.86-1.73 (m, 2H), 1.45 (s, 9H).

LC/MS (Method 1, ESIneg): Rₜ=2.45 min, m/z=475.20 [M−H]⁻.

### Example 198A

### tert-Butyl 2-{[3-cyclopentyl-5-methyl-2,4-dioxo-1-(3,3,3-trifluoropropyl)-1,2,3,4-tetrahydrothieno[2,3-d]pyrimidin-6-yl]methylene}hydrazinecarboxylate

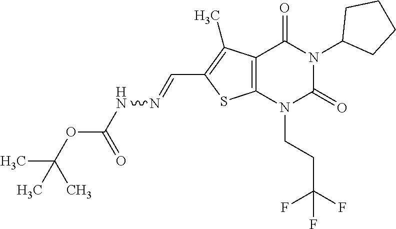 [SMILES: Cc1c(C=NNC(=O)OC(C)(C)C)sc2c1c(=O)n(C1CCCC1)c(=O)n2CCC(F)(F)F]

Analogously to the method described in Ex. 156A, 400 mg (1.07 mmol) of the compound from Ex. 98A and 212 mg (1.60 mmol) of tert-butyl hydrazinecarboxylate were used to prepare 477 mg (83% of theory, 91% purity) of the title compound, which was used for subsequent reactions without further purification.

¹H-NMR (400 MHz, DMSO-d₆, δ/ppm): 10.87 (br. s, 1H), 8.30 (s, 1H), 5.29 (quin, 1H), 4.12 (t, 2H), 2.85-2.72 (m, 2H), 2.45 (s, 3H), 2.08-1.97 (m, 2H), 1.95-1.83 (m, 2H), 1.81-1.69 (m, 2H), 1.63-1.49 (m, 2H), 1.45 (s, 9H).

LC/MS (Method 2, ESIneg): Rₜ=1.27 min, m/z=487 [M−H]⁻.

### Example 199A

### tert-Butyl 2-{[3-cyclopentyl-1-(2-methoxyethyl)-5-methyl-2,4-dioxo-1,2,3,4-tetrahydrothieno[2,3-d]pyrimidin-6-yl]methylene}hydrazinecarboxylate

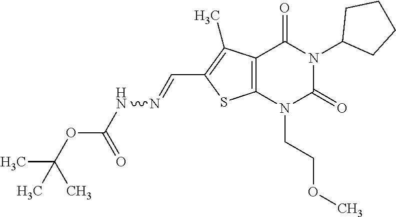 [SMILES: COCCn1c(=O)n(C2CCCC2)c(=O)c2c(C)c(C=NNC(=O)OC(C)(C)C)sc21]

Analogously to the method described in Ex. 156A, 400 mg (1.19 mmol) of the compound from Ex. 99A and 236 mg (1.78 mmol) of tert-butyl hydrazinecarboxylate were used to prepare 505 mg (88% of theory, 93% purity) of the title compound, which was used for subsequent reactions without further purification.

¹H-NMR (400 MHz, DMSO-d₆, δ/ppm): 10.84 (br. s, 1H), 8.28 (s, 1H), 5.29 (quin, 1H), 4.05 (t, 2H), 3.64 (t, 2H), 3.25 (s, 3H), 2.44 (s, 3H), 2.09-1.96 (m, 2H), 1.94-1.82 (m, 2H), 1.80-1.69 (m, 2H), 1.62-1.49 (m, 2H), 1.45 (s, 9H).

LC/MS (Method 2, ESIneg): Rₜ=1.17 min, m/z=449 [M−H]⁻.

### Example 200A

### tert-Butyl 2-[(3-cyclopropyl-1,5-dimethyl-2,4-dioxo-1,2,3,4-tetrahydrothieno[2,3-d]pyrimidin-6-yl)methyl]hydrazinecarboxylate

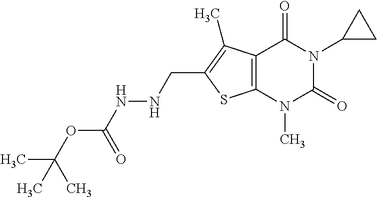 [SMILES: Cc1c(CNNC(=O)OC(C)(C)C)sc2c1c(=O)n(C1CC1)c(=O)n2C]

To a solution of 325 mg (0.859 mmol) of the compound from Ex. 156A in 18 ml of methanol were added 270 mg (4.29 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 135 mg (2.15 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP KP-Sil cartridge, 50 g of silica gel, eluent: cyclohexane/ethyl acetate 2:1). The product fractions were combined and concentrated. Drying under high vacuum gave 232 mg (71% of theory) of the title compound.

¹H-NMR (400 MHz, DMSO-d₆, δ/ppm): 8.24 (br. s, 1H), 4.96 (br. s, 1H), 3.96 (br. d, 2H), 3.38 (s, 3H), 2.62-2.56 (m, 1H), 2.31 (s, 3H), 1.38 (s, 9H), 1.06-0.96 (m, 2H), 0.71-0.63 (m, 2H).

LC/MS (Method 1, ESIneg): Rₜ=1.53 min, m/z=379.14 [M−H]⁻.

### Example 201A

### tert-Butyl 2-[(1-butyl-3-cyclopropyl-5-methyl-2,4-dioxo-1,2,3,4-tetrahydrothieno[2,3-d]pyrimidin-6-yl)methyl]hydrazinecarboxylate

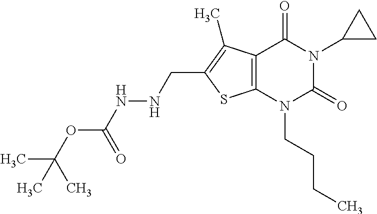 [SMILES: CCCCn1c(=O)n(C2CC2)c(=O)c2c(C)c(CNNC(=O)OC(C)(C)C)sc21]

Analogously to the method described in Ex. 200A, 400 mg (0.951 mmol) of the compound from Ex. 157A and a total of 448 mg (7.13 mmol) of sodium cyanoborohydride were used to prepare 318 mg (72% of theory, 92% purity) of the title compound, which was used for subsequent reactions without further purification.

¹H-NMR (400 MHz, DMSO-d₆, δ/ppm): 8.23 (br. s, 1H), 4.99 (br. d, 1H), 3.96 (br. d, 2H), 3.83 (t, 2H), 2.63-2.56 (m, 1H), 2.31 (s, 3H), 1.65 (quin, 2H), 1.38 (s, 9H), 1.38-1.29 (m, 2H), 1.05-0.96 (m, 2H), 0.92 (t, 3H), 0.71-0.61 (m, 2H).

LC/MS (Method 1, ESIneg): Rₜ=1.98 min, m/z=421.19 [M−H]⁻.

### Example 202A

### tert-Butyl 2-{[3-cyclopropyl-5-methyl-2,4-dioxo-1-(3,3,3-trifluoropropyl)-1,2,3,4-tetrahydrothieno[2,3-d]pyrimidin-6-yl]methyl}hydrazinecarboxylate

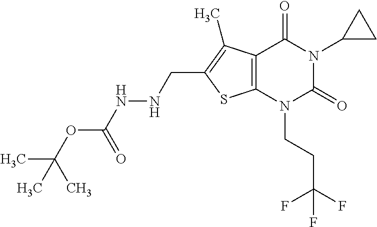 [SMILES: Cc1c(CNNC(=O)OC(C)(C)C)sc2c1c(=O)n(C1CC1)c(=O)n2CCC(F)(F)F]

To a solution of 655 mg (1.42 mmol) of the compound from Ex. 158A in 30 ml of methanol were added 447 mg (7.11 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h and after 3 h, a further 224 mg (3.55 mmol) of sodium cyanoborohydride were added in each case. Over the entire reaction time, by addition of further acetic acid, the pH was constantly regulated such that the indicator colour just remained yellow. After a total of 7 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP KP-Sil cartridge, 50 g of silica gel, eluent: cyclohexane/ethyl acetate 1:2). The product fractions were combined and concentrated. Drying under high vacuum gave 570 mg (86% of theory) of the title compound.

¹H-NMR (400 MHz, DMSO-d₆, δ/ppm): 8.24 (br. s, 1H), 5.04 (br. d, 1H), 4.08 (t, 2H), 3.97 (d, 2H), 2.82-2.68 (m, 2H), 2.64-2.56 (m, 1H), 2.31 (s, 3H), 1.38 (s, 9H), 1.06-0.96 (m, 2H), 0.70-0.62 (m, 2H).

LC/MS (Method 1, ESIneg): Rₜ=1.86 min, m/z=461.15 [M−H]⁻.

### Example 203A

### tert-Butyl 2-{[1-(cyclobutylmethyl)-3-cyclopropyl-5-methyl-2,4-dioxo-1,2,3,4-tetrahydrothieno[2,3-d]pyrimidin-6-yl]methyl}hydrazinecarboxylate

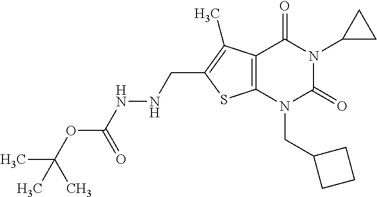 [SMILES: Cc1c(CNNC(=O)OC(C)(C)C)sc2c1c(=O)n(C1CC1)c(=O)n2CC1CCC1]

Analogously to the method described in Ex. 200A, 350 mg (0.809 mmol) of the compound from Ex. 159A and a total of 381 mg (6.07 mmol) of sodium cyanoborohydride were used to prepare 378 mg (97% of theory, 90% purity) of the title compound, which was used for subsequent reactions without further purification.

¹H-NMR (400 MHz, DMSO-d₆, δ/ppm): 8.24 (br. s, 1H), 4.99 (br. d, 1H), 3.95 (br. d, 2H), 3.90 (d, 2H), 2.85-2.71 (m, 1H), 2.59 (tt, 1H), 2.30 (s, 3H), 2.03-1.91 (m, 2H), 1.89-1.74 (m, 4H), 1.38 (s, 9H), 1.03-0.98 (m, 2H), 0.69-0.60 (m, 2H).

LC/MS (Method 1, ESIpos): Rₜ=2.02 min, m/z=303.12 [M+H−C₅H₁₂N₂O₂]⁺.

### Example 204A

### tert-Butyl 2-({3-cyclopropyl-1-[(2,2-difluorocyclopropyl)methyl]-5-methyl-2,4-dioxo-1,2,3,4-tetrahydrothieno[2,3-d]pyrimidin-6-yl}methyl)hydrazinecarboxylate (racemate)

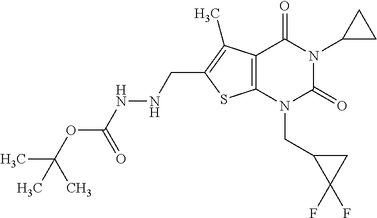 [SMILES: Cc1c(CNNC(=O)OC(C)(C)C)sc2c1c(=O)n(C1CC1)c(=O)n2CC1CC1(F)F]

Analogously to the method described in Ex. 200A, 740 mg (1.63 mmol) of the compound from Ex. 160A and a total of 767 mg (12.2 mmol) of sodium cyanoborohydride were used to prepare 690 mg (80% of theory, 86% purity) of the title compound, which was used for subsequent reactions without further purification.

¹H-NMR (400 MHz, DMSO-d₆, δ/ppm): 8.27 (br. s, 1H), 5.03 (br. s, 1H), 4.10-3.89 (m, 2H), 3.97 (s, 2H), 2.65-2.57 (m, 1H), 2.31 (s, 3H), 2.20 (td, 1H), 1.76-1.62 (m, 1H), 1.53-1.42 (m, 1H), 1.38 (s, 9H), 1.06-0.98 (m, 2H), 0.71-0.64 (m, 2H).

LC/MS (Method 1, ESIneg): Rₜ=1.87 min, m/z=455.16 [M−H]⁻.

### Example 205A

### tert-Butyl 2-{[1-(cyclomethyl)-3-cyclopropyl-5-methyl-2,4-dioxo-1,2,3,4-tetrahydrothieno[2,3-d]pyrimidin-6-yl]methyl}hydrazinecarboxylate

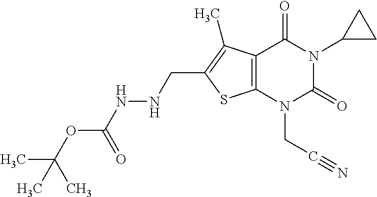 [SMILES: Cc1c(CNNC(=O)OC(C)(C)C)sc2c1c(=O)n(C1CC1)c(=O)n2CC#N]

Analogously to the method described in Ex. 200A, 305 mg (0.756 mmol) of the compound from Ex. 161A and a total of 356 mg (5.67 mmol) of sodium cyanoborohydride were used to prepare 222 mg (80% of theory) of the title compound.

¹H-NMR (400 MHz, DMSO-d₆, δ/ppm): 8.27 (br. s, 1H), 5.06 (s, 2H), 5.04 (br. s, 1H), 3.99 (br. d, 2H), 2.67-2.59 (m, 1H), 2.32 (s, 3H), 1.39 (s, 9H), 1.07-0.96 (m, 2H), 0.74-0.65 (m, 2H).

LC/MS (Method 1, ESIneg): Rₜ=1.58 min, m/z=404.14 [M−H]⁻.

### Example 206A

### tert-Butyl 2-{[3-cyclopropyl-1-(2-methoxyethyl)-5-methyl-2,4-dioxo-1,2,3,4-tetrahydrothieno[2,3-d]pyrimidin-6-yl]methyl}hydrazinecarboxylate

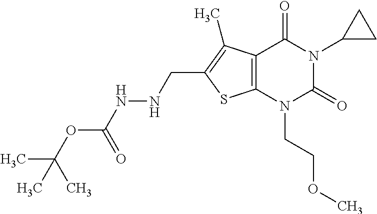 [SMILES: COCCn1c(=O)n(C2CC2)c(=O)c2c(C)c(CNNC(=O)OC(C)(C)C)sc21]

Analogously to the method described in Ex. 202A, 305 mg (0.756 mmol) of the compound from Ex. 162A and a total of 736 mg (2.34 mmol) of sodium cyanoborohydride were used to prepare 369 mg (72% of theory) of the title compound. The total reaction time in this case was about 20 h.

¹H-NMR (400 MHz, DMSO-d₆, δ/ppm): 8.24 (br. s, 1H), 4.96 (br. d, 1H), 3.99 (t, 2H), 3.95 (br. d, 2H), 3.62 (t, 2H), 3.25 (s, 3H), 2.64-2.55 (m, 1H), 2.31 (s, 3H), 1.38 (s, 9H), 1.05-0.96 (m, 2H), 0.71-0.61 (m, 2H).

LC/MS (Method 1, ESIneg): Rₜ=1.58 min, m/z=423.17 [M−H]⁻.

### Example 207A

### tert-Butyl 2-{[3-cyclopropyl-1-(2-ethoxyethyl)-5-methyl-2,4-dioxo-1,2,3,4-tetrahydrothieno[2,3-d]pyrimidin-6-yl]methyl}hydrazinecarboxylate

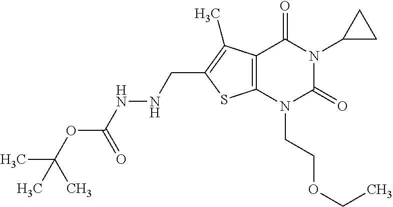 [SMILES: CCOCCn1c(=O)n(C2CC2)c(=O)c2c(C)c(CNNC(=O)OC(C)(C)C)sc21]

Analogously to the method described in Ex. 200A, 325 mg (0.745 mmol) of the compound from Ex. 163A and a total of 227 mg (3.61 mmol) of sodium cyanoborohydride were used to prepare 233 mg (71% of theory) of the title compound.

¹H-NMR (400 MHz, DMSO-d₆, δ/ppm): 8.23 (br. s, 1H), 4.96 (br. s, 1H), 3.98 (t, 2H), 3.96 (br. d, 2H), 3.65 (t, 2H), 3.45 (q, 2H), 2.64-2.56 (m, 1H), 2.31 (s, 3H), 1.38 (s, 9H), 1.06 (t, 3H), 1.04-0.98 (m, 2H), 0.71-0.60 (m, 2H).

LC/MS (Method 1, ESIpos): Rₜ=1.73 min, m/z=307.11 [M+H−C₅H₁₂N₂O₂]⁺.

### Example 208A

### tert-Butyl 2-{[3-cyclopropyl-1-(2-isopropoxyethyl)-5-methyl-2,4-dioxo-1,2,3,4-tetrahydrothieno[2,3-d]pyrimidin-6-yl]methyl}hydrazinecarboxylate

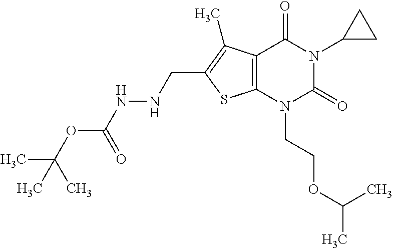 [SMILES: Cc1c(CNNC(=O)OC(C)(C)C)sc2c1c(=O)n(C1CC1)c(=O)n2CCOC(C)C]

Analogously to the method described in Ex. 200A, 380 mg (0.843 mmol) of the compound from Ex. 164A and a total of 227 mg (3.61 mmol) of sodium cyanoborohydride were used to prepare 377 mg (98% of theory) of the title compound.

¹H-NMR (400 MHz, DMSO-d₆, δ/ppm): 8.22 (br. s, 1H), 4.95 (br. d, 1H), 4.01-3.88 (m, 4H), 3.64 (t, 2H), 3.56 (dt, 1H), 2.60 (tt, 1H), 2.31 (s, 3H), 1.38 (s, 9H), 1.04-0.98 (m, 2H), 1.03 (d, 6H), 0.71-0.60 (m, 2H).

LC/MS (Method 2, ESIneg): Rₜ=0.98 min, m/z=497 [M−H+HCOOH]⁻.

### Example 209A

### tert-Butyl 2-({3-cyclopropyl-5-methyl-2,4-dioxo-1-[2-(trifluoromethoxy)ethyl]-1,2,3,4-tetrahydrothieno[2,3-d]pyrimidin-6-yl}methyl)hydrazinecarboxylate

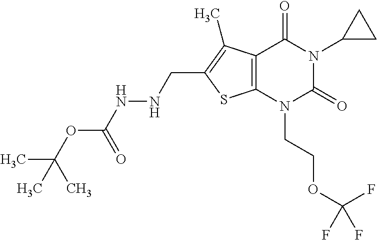 [SMILES: Cc1c(CNNC(=O)OC(C)(C)C)sc2c1c(=O)n(C1CC1)c(=O)n2CCOC(F)(F)F]

To a solution of 440 mg (0.923 mmol) of the compound from Ex. 165A in 20 ml of methanol were added 290 mg (4.62 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 145 mg (2.31 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP KP-Sil cartridge, 50 g of silica gel, eluent: cyclohexane/ethyl acetate 2:1). The product fractions were combined and concentrated. Drying under high vacuum gave 339 mg (72% of theory, 95% purity) of the title compound, which was used for subsequent reactions without further purification.

¹H-NMR (400 MHz, DMSO-d₆, δ/ppm): 8.23 (br. s, 1H), 4.98 (br. d, 1H), 4.39 (t, 2H), 4.16 (t, 2H), 3.96 (br. d, 2H), 2.65-2.57 (m, 1H), 2.31 (s, 3H), 1.38 (s, 9H), 1.04-0.99 (m, 2H), 0.70-0.61 (m, 2H).

LC/MS (Method 1, ESIneg): Rₜ=1.90 min, m/z=477.14 [M−H]⁻.

### Example 210A

### tert-Butyl 2-{[3-cyclopropyl-5-methyl-2,4-dioxo-1-(tetrahydrofuran-2-ylmethyl)-1,2,3,4-tetrahydrothieno[2,3-d]pyrimidin-6-yl]methyl}hydrazinecarboxylate (racemate)

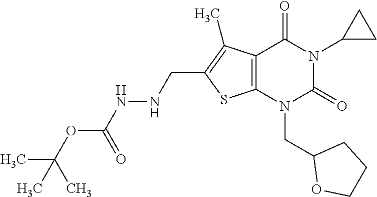 [SMILES: Cc1c(CNNC(=O)OC(C)(C)C)sc2c1c(=O)n(C1CC1)c(=O)n2CC1CCCO1]

Analogously to the method described in Ex. 202A, 805 mg (1.80 mmol) of the compound from Ex. 166A and a total of 959 mg (15.3 mmol) of sodium cyanoborohydride were used to prepare 619 mg (76% of theory) of the title compound.

¹H-NMR (400 MHz, DMSO-d₆, δ/ppm): 8.24 (br. s, 1H), 4.95 (br. s, 1H), 4.29-4.18 (m, 1H), 4.02-3.88 (m, 3H), 3.80-3.74 (m, 1H), 3.70 (dd, 1H), 3.65-3.57 (m, 1H), 2.60 (td, 1H), 2.31 (s, 3H), 2.01-1.76 (m, 3H), 1.72-1.60 (m, 1H), 1.39 (s, 9H), 1.07-0.96 (m, 2H), 0.70-0.62 (m, 2H).

LC/MS (Method 1, ESIneg): Rₜ=1.69 min, m/z=449.19 [M−H]⁻.

### Example 211A

### tert-Butyl 2-({3-cyclopropyl-5-methyl-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

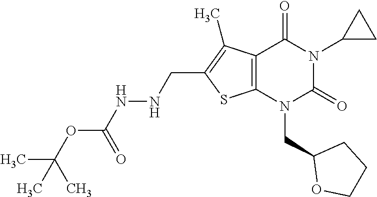 [SMILES: Cc1c(CNNC(=O)OC(C)(C)C)sc2c1c(=O)n(C1CC1)c(=O)n2C[C@H]1CCCO1]

Analogously to the method described in Ex. 200A, 275 mg (0.613 mmol) of the compound from Ex. 167A and a total of 289 mg (4.60 mmol) of sodium cyanoborohydride were used to prepare 223 mg (80% of theory) of the title compound.

¹H-NMR (400 MHz, DMSO-d₆, δ/ppm): 8.24 (br. s, 1H), 4.95 (br. s, 1H), 4.32-4.17 (m, 1H), 4.03-3.89 (m, 3H), 3.81-3.74 (m, 1H), 3.70 (dd, 1H), 3.65-3.57 (m, 1H), 2.60 (tt, 1H), 2.31 (s, 3H), 2.03-1.75 (m, 3H), 1.72-1.60 (m, 1H), 1.39 (s, 9H), 1.06-0.95 (m, 2H), 0.71-0.60 (m, 2H).

LC/MS (Method 1, ESIneg): Rₜ=1.70 min, m/z=449.19 [M−H]⁻.

### Example 212A

### tert-Butyl 2-({3-cyclopropyl-5-methyl-2,4-dioxo-1-[(2S)-tetrahydrofuran-2-ylmethyl]-1,2,3,4-tetrahydrothieno[2,3-d]pyrimidin-6-yl}methyl)hydrazinecarboxylate

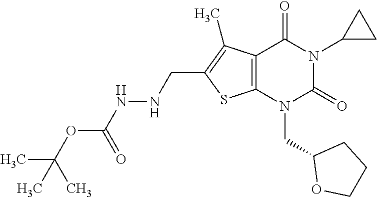 [SMILES: Cc1c(CNNC(=O)OC(C)(C)C)sc2c1c(=O)n(C1CC1)c(=O)n2C[C@@H]1CCCO1]

Analogously to the method described in Ex. 200A, 235 mg (0.524 mmol) of the compound from Ex. 168A and a total of 247 mg (3.93 mmol) of sodium cyanoborohydride were used to prepare 178 mg (74% of theory) of the title compound.

¹H-NMR (400 MHz, DMSO-d₆, δ/ppm): 8.24 (br. s, 1H), 4.95 (br. s, 1H), 4.31-4.16 (m, 1H), 4.04-3.89 (m, 3H), 3.81-3.74 (m, 1H), 3.70 (dd, 1H), 3.65-3.57 (m, 1H), 2.64-2.56 (m, 1H), 2.31 (s, 3H), 2.03-1.74 (m, 3H), 1.72-1.59 (m, 1H), 1.39 (s, 9H), 1.07-0.94 (m, 2H), 0.72-0.60 (m, 2H).

LC/MS (Method 1, ESIneg): Rₜ=1.70 min, m/z=449.19 [M−H]⁻.

### Example 213A

### tert-Butyl 2-{[1,5-dimethyl-3-(1-methylcyclopropyl)-2,4-dioxo-1,2,3,4-tetrahydrothieno[2,3-d]pyrimidin-6-yl]methyl}hydrazinecarboxylate

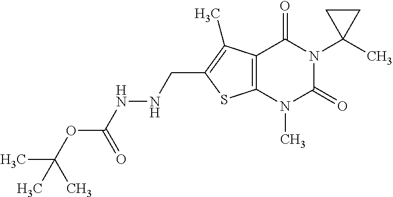 [SMILES: Cc1c(CNNC(=O)OC(C)(C)C)sc2c1c(=O)n(C1(C)CC1)c(=O)n2C]

Analogously to the method described in Ex. 200A, 355 mg (0.905 mmol) of the compound from Ex. 169A and a total of 426 mg (6.78 mmol) of sodium cyanoborohydride were used to prepare 267 mg (71% of theory, 95% purity) of the title compound, which was used for subsequent reactions without further purification.

¹H-NMR (400 MHz, DMSO-d₆, δ/ppm): 8.23 (br. s, 1H), 4.96 (br. d, 1H), 3.96 (d, 2H), 3.38 (s, 3H), 2.32 (s, 3H), 1.38 (s, 9H), 1.33 (s, 3H), 0.99-0.76 (m, 4H).

LC/MS (Method 2, ESIneg): Rₜ=0.89 min, m/z=439 [M−H+HCOOH]⁻.

### Example 214A

### tert-Butyl 2-{[1-butyl-5-methyl-3-(1-methylcyclopropyl)-2,4-dioxo-1,2,3,4-tetrahydrothieno[2,3-d]pyrimidin-6-yl]methyl}hydrazinecarboxylate

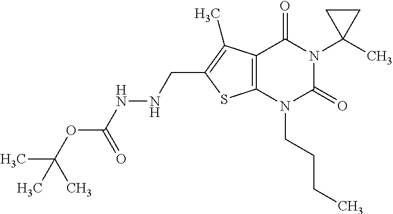 [SMILES: CCCCn1c(=O)n(C2(C)CC2)c(=O)c2c(C)c(CNNC(=O)OC(C)(C)C)sc21]

Analogously to the method described in Ex. 200A, 392 mg (0.902 mmol) of the compound from Ex. 170A and a total of 253 mg (4.03 mmol) of sodium cyanoborohydride were used to prepare 353 mg (89% of theory) of the title compound.

¹H-NMR (400 MHz, DMSO-d₆, δ/ppm): 8.22 (br. s, 1H), 4.98 (br. s, 1H), 3.95 (br. d, 2H), 3.90-3.70 (m, 2H), 2.31 (s, 3H), 1.65 (quin, 2H), 1.44-1.23 (m, 2H), 1.38 (s, 9H), 1.33 (s, 3H), 1.02-0.66 (m, 2H), 0.92 (t, 3H).

LC/MS (Method 1, ESIneg): Rₜ=2.14 min, m/z=435.21 [M−H]⁻.

### Example 215A

### tert-Butyl 2-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methyl}hydrazinecarboxylate

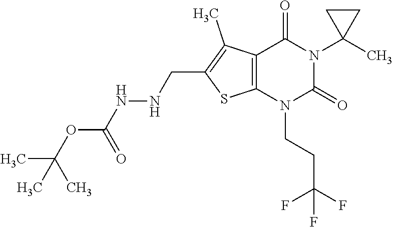 [SMILES: Cc1c(CNNC(=O)OC(C)(C)C)sc2c1c(=O)n(C1(C)CC1)c(=O)n2CCC(F)(F)F]

To a solution of 1.33 g (2.64 mmol, 94% purity) of the compound from Ex. 171A in 25 ml of methanol were added 830 mg (13.21 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 16 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 1.28 g (80% of theory, 79% purity) of the title compound were obtained, which was used for subsequent reactions without further purification.

LC/MS (Method 2, ESIneg): Rₜ=1.05 min, m/z=521 [M−H+HCOOH]⁻.

### Example 216A

### tert-Butyl 2-{[1-(cyclobutylmethyl)-5-methyl-3-(1-methylcyclopropyl)-2,4-dioxo-1,2,3,4-tetrahydrothieno[2,3-d]pyrimidin-6-yl]methyl}hydrazinecarboxylate

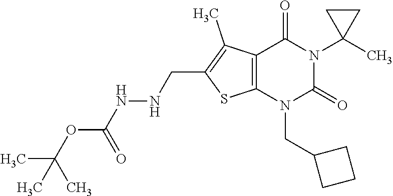 [SMILES: Cc1c(CNNC(=O)OC(C)(C)C)sc2c1c(=O)n(C1(C)CC1)c(=O)n2CC1CCC1]

Analogously to the method described in Ex. 200A, 360 mg (0.806 mmol) of the compound from Ex. 172A and a total of 380 mg (6.05 mmol) of sodium cyanoborohydride were used to prepare 249 mg (68% of theory) of the title compound.

¹H-NMR (400 MHz, DMSO-d₆, δ/ppm): 8.23 (br. s, 1H), 4.98 (br. d, 1H), 4.05-3.73 (m, 2H), 3.95 (d, 2H), 2.83-2.72 (m, 1H), 2.31 (s, 3H), 2.03-1.90 (m, 2H), 1.89-1.75 (m, 4H), 1.38 (s, 9H), 1.32 (s, 3H), 1.00-0.66 (m, 4H).

LC/MS (Method 1, ESIneg): Rₜ=2.19 min, m/z=447.21 [M−H]⁻.

### Example 217A

### tert-Butyl 2-({1-[(2,2-difluorocyclopropyl)methyl]-5-methyl-3-(1-methylcyclopropyl)-2,4-dioxo-1,2,3,4-tetrahydrothieno[2,3-d]pyrimidin-6-yl}methyl)hydrazinecarboxylate (racemate)

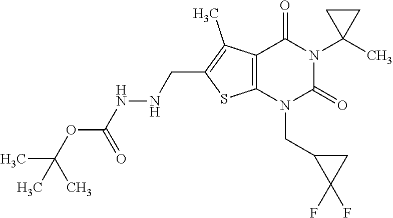 [SMILES: Cc1c(CNNC(=O)OC(C)(C)C)sc2c1c(=O)n(C1(C)CC1)c(=O)n2CC1CC1(F)F]

Analogously to the method described in Ex. 200A, 810 mg (1.73 mmol) of the compound from Ex. 173A and a total of 485 mg (7.72 mmol) of sodium cyanoborohydride were used to prepare 682 mg (76% of theory, 91% purity) of the title compound, which was used for subsequent reactions without further purification.

¹H-NMR (400 MHz, DMSO-d₆, δ/ppm): 8.23 (br. s, 1H), 5.01 (br. s, 1H), 4.18-3.84 (m, 2H), 3.97 (br. d, 2H), 2.32 (s, 3H), 2.27-2.12 (m, 1H), 1.78-1.62 (m, 1H), 1.52-1.42 (m, 1H), 1.38 (s, 9H), 1.34 (s, 3H), 1.00-0.75 (m, 4H).

LC/MS (Method 1, ESIneg): Rₜ=2.00 min, m/z=469.17 [M−H]⁻.

### Example 218A

### tert-Butyl 2-{[1-(cyanomethyl)-5-methyl-3-(1-methylcyclopropyl)-2,4-dioxo-1,2,3,4-tetrahydrothieno[2,3-d]pyrimidin-6-yl]methyl}hydrazinecarboxylate

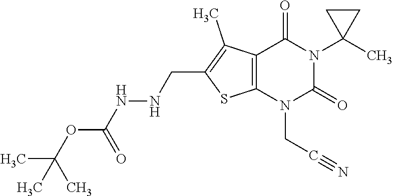 [SMILES: Cc1c(CNNC(=O)OC(C)(C)C)sc2c1c(=O)n(C1(C)CC1)c(=O)n2CC#N]

Analogously to the method described in Ex. 200A, 285 mg (0.478 mmol, 70% purity) of the compound from Ex. 174A and a total of 225 mg (3.58 mmol) of sodium cyanoborohydride were used to prepare 204 mg (94% of theory, 92% purity) of the title compound, which was used for subsequent reactions without further purification.

¹H-NMR (400 MHz, DMSO-d₆, δ/ppm): 8.26 (br. s, 1H), 5.09-5.01 (m, 3H), 3.99 (br. d, 2H), 2.33 (s, 3H), 1.39 (s, 9H), 1.35 (s, 3H), 1.02-0.77 (m, 4H).

LC/MS (Method 2, ESIneg): Rₜ=0.90 min, m/z=418 [M−H]⁻.

### Example 219A

### tert-Butyl 2-{[1-(2-methoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

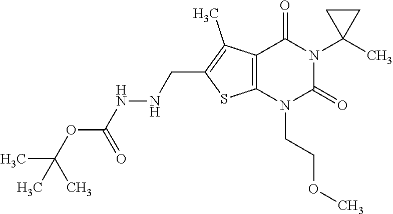 [SMILES: COCCn1c(=O)n(C2(C)CC2)c(=O)c2c(C)c(CNNC(=O)OC(C)(C)C)sc21]

To a solution of 1.17 g (1.98 mmol, 73% purity) of the compound from Ex. 175A in 19 ml of methanol were added 620 mg (9.87 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 2 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 660 mg (69% of theory, 91% purity) of the title compound were obtained, which was used for subsequent reactions without further purification.

LC/MS (Method 2, ESIpos): Rₜ=0.91 min, m/z=439 [M+H]⁺.

### Example 220A

### tert-Butyl 2-({5-methyl-3-(1-methylcyclopropyl)-2,4-dioxo-1-[2-(trifluoromethoxy)ethyl]-1,2,3,4-tetrahydrothieno[2,3-d]pyrimidin-6-yl}methyl)hydrazinecarboxylate

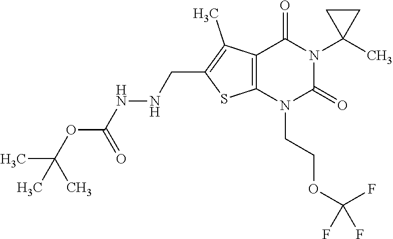 [SMILES: Cc1c(CNNC(=O)OC(C)(C)C)sc2c1c(=O)n(C1(C)CC1)c(=O)n2CCOC(F)(F)F]

To a solution of 420 mg (0.685 mmol, 80% purity) of the compound from Ex. 176A in 15 ml of methanol were added 215 mg (3.43 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 108 mg (1.72 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP KP-Sil cartridge, 50 g of silica gel, eluent: cyclohexane/ethyl acetate 2:1). The product fractions were combined and concentrated. Drying under high vacuum gave 292 mg (74% of theory, 86% purity) of the title compound, which was used for subsequent reactions without further purification.

¹H-NMR (400 MHz, DMSO-d₆, δ/ppm): 8.23 (br. s, 1H), 4.98 (br. d, 1H), 4.46-4.33 (m, 2H), 4.30-4.04 (m, 2H), 3.96 (br. d, 2H), 2.32 (s, 3H), 1.38 (s, 9H), 1.33 (s, 3H), 0.97-0.75 (m, 4H).

LC/MS (Method 1, ESIneg): Rₜ=2.03 min, m/z=537.16 [M−H+HCOOH]⁻.

### Example 221A

### tert-Butyl 2-{[1-(2-ethoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

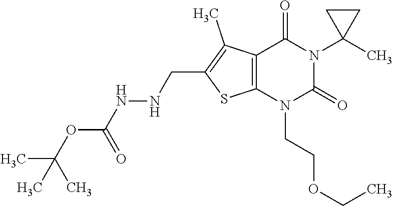 [SMILES: CCOCCn1c(=O)n(C2(C)CC2)c(=O)c2c(C)c(CNNC(=O)OC(C)(C)C)sc21]

Analogously to the method described in Ex. 200A, 310 mg (0.482 mmol, 70% purity) of the compound from Ex. 177A and a total of 227 mg (3.61 mmol) of sodium cyanoborohydride were used to prepare 202 mg (76% of theory, 82% purity) of the title compound, which was used for subsequent reactions without further purification.

¹H-NMR (400 MHz, DMSO-d₆, δ/ppm): 8.22 (br. s, 1H), 4.96 (br. d, 1H), 4.15-3.80 (m, 2H), 3.95 (br. d, 2H), 3.65 (t, 2H), 3.45 (q, 2H), 2.31 (s, 3H), 1.38 (s, 9H), 1.33 (s, 3H), 1.05 (t, 3H), 0.96-0.76 (m, 4H).

LC/MS (Method 1, ESIneg): Rₜ=1.88 min, m/z=451.20 [M−H]⁻.

### Example 222A

### tert-Butyl 2-{[1-(2-isopropoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

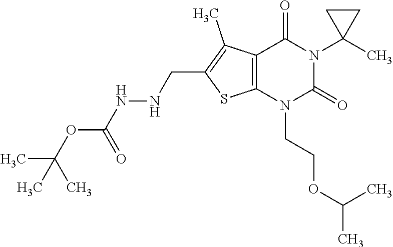 [SMILES: Cc1c(CNNC(=O)OC(C)(C)C)sc2c1c(=O)n(C1(C)CC1)c(=O)n2CCOC(C)C]

Analogously to the method described in Ex. 200A, 365 mg (0.786 mmol) of the compound from Ex. 178A and a total of 227 mg (3.61 mmol) of sodium cyanoborohydride were used to prepare 284 mg (77% of theory) of the title compound.

¹H-NMR (400 MHz, DMSO-d₆, δ/ppm): 8.21 (br. s, 1H), 4.95 (br. d, 1H), 4.08-3.82 (m, 2H), 3.95 (br. d, 2H), 3.64 (t, 2H), 3.56 (sept, 1H), 2.31 (s, 3H), 1.38 (s, 9H), 1.33 (s, 3H), 1.03 (d, 6H), 0.96-0.74 (m, 4H).

LC/MS (Method 1, ESIneg): Rₜ=2.01 min, m/z=465.22 [M−H]⁻.

### Example 223A

### tert-Butyl 2-({5-methyl-3-(1-methylcyclopropyl)-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

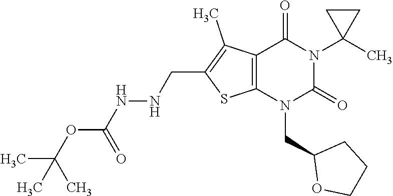 [SMILES: Cc1c(CNNC(=O)OC(C)(C)C)sc2c1c(=O)n(C1(C)CC1)c(=O)n2C[C@H]1CCCO1]

Analogously to the method described in Ex. 200A, 235 mg (0.508 mmol) of the compound from Ex. 179A and a total of 239 mg (3.81 mmol) of sodium cyanoborohydride were used to prepare 152 mg (64% of theory) of the title compound.

¹H-NMR (400 MHz, DMSO-d₆, δ/ppm): 8.23 (br. s, 1H), 4.94 (br. s, 1H), 4.32-3.85 (m, 4H), 3.84-3.53 (m, 3H), 2.31 (s, 3H), 2.02-1.76 (m, 3H), 1.72-1.60 (m, 1H), 1.38 (s, 9H), 1.33 (s, 3H), 0.97-0.71 (m, 4H).

LC/MS (Method 1, ESIneg): Rₜ=1.86 min, m/z=509.21 [M−H+HCOOH]⁻.

### Example 224A

### tert-Butyl 2-({5-methyl-3-(1-methylcyclopropyl)-2,4-dioxo-1-[(2S)-tetrahydrofuran-2-ylmethyl]-1,2,3,4-tetrahy drothieno[2,3-d]pyrimidin-6-yl}methyl)hydrazinecarboxylate

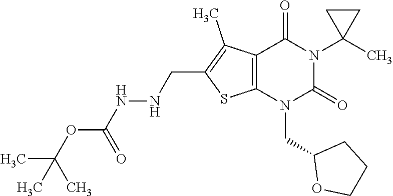 [SMILES: Cc1c(CNNC(=O)OC(C)(C)C)sc2c1c(=O)n(C1(C)CC1)c(=O)n2C[C@@H]1CCCO1]

Analogously to the method described in Ex. 200A, 340 mg (0.735 mmol) of the compound from Ex. 180A and a total of 346 mg (5.51 mmol) of sodium cyanoborohydride were used to prepare 270 mg (79% of theory) of the title compound.

¹H-NMR (400 MHz, DMSO-d₆, δ/ppm): 8.23 (br. s, 1H), 4.94 (br. s, 1H), 4.30-3.85 (m, 4H), 3.83-3.53 (m, 3H), 2.31 (s, 3H), 2.03-1.75 (m, 3H), 1.73-1.59 (m, 1H), 1.38 (s, 9H), 1.33 (s, 3H), 0.98-0.72 (m, 4H).

LC/MS (Method 1, ESIneg): Rₜ=1.86 min, m/z=509.21 [M−H+HCOOH]⁻.

### Example 225A

### tert-Butyl 2-({1-(2-methoxyethyl)-5-methyl-2,4-dioxo-3-[1-(trifluoromethyl)cyclopropyl]-1,2,3,4-tetrahydrothieno[2,3-d]pyrimidin-6-yl}methyl)hydrazinecarboxylate

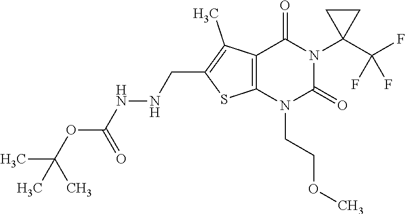 [SMILES: COCCn1c(=O)n(C2(C(F)(F)F)CC2)c(=O)c2c(C)c(CNNC(=O)OC(C)(C)C)sc21]

Analogously to the method described in Ex. 200A, 445 mg (0.907 mmol) of the compound from Ex. 181A and a total of 428 mg (6.80 mmol) of sodium cyanoborohydride were used to prepare 405 mg (87% of theory, 96% of theory) of the title compound.

¹H-NMR (400 MHz, DMSO-d₆, δ/ppm): 8.24 (br. s, 1H), 5.00 (br. d, 1H), 4.05-3.99 (m, 2H), 3.97 (br. d, 2H), 3.63 (t, 2H), 3.25 (s, 3H), 2.31 (s, 3H), 1.65-1.51 (m, 2H), 1.38 (s, 9H), 1.35-1.31 (m, 2H).

LC/MS (Method 1, ESIneg): Rₜ=1.88 min, m/z=491.16 [M−H]⁻.

### Example 226A

### tert-Butyl 2-{[3-(1-ethylcyclopropyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

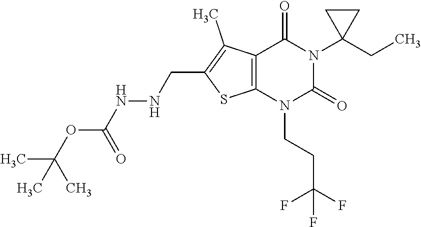 [SMILES: CCC1(n2c(=O)c3c(C)c(CNNC(=O)OC(C)(C)C)sc3n(CCC(F)(F)F)c2=O)CC1]

Analogously to the method described in Ex. 200A, 200 mg (0.328 mmol, 80% purity) of the compound from Ex. 182A and a total of 154 mg (2.46 mmol) of sodium cyanoborohydride were used to prepare 146 mg (90% of theory) of the title compound.

LC/MS (Method 1, ESIneg): Rₜ=2.17 min, m/z=535.18 [M−H+HCOOH]⁻.

### Example 227A

### tert-Butyl 2-{[3-(1-ethylcyclopropyl)-1-(2-methoxyethyl)-5-methyl-2,4-dioxo-1,2,3,4-tetrahydrothieno[2,3-d]pyrimidin-6-yl]methyl}hydrazinecarboxylate

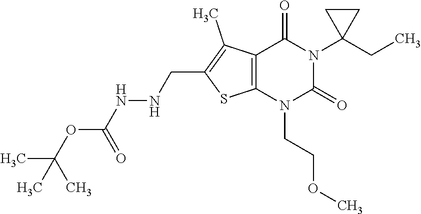 [SMILES: CCC1(n2c(=O)c3c(C)c(CNNC(=O)OC(C)(C)C)sc3n(CCOC)c2=O)CC1]

Analogously to the method described in Ex. 200A, 270 mg (0.527 mmol, 88% purity) of the compound from Ex. 183A and a total of 249 mg (3.96 mmol) of sodium cyanoborohydride were used to prepare 146 mg (61% of theory) of the title compound.

LC/MS (Method 1, ESIneg): Rₜ=1.91 min, m/z=451.20 [M−H]⁻.

### Example 228A

### tert-Butyl 2-{[3-cyclobutyl-5-methyl-2,4-dioxo-1-(3,3,3-trifluoropropyl)-1,2,3,4-tetrahydrothieno[2,3-d]pyrimidin-6-yl]methyl}hydrazinecarboxylate

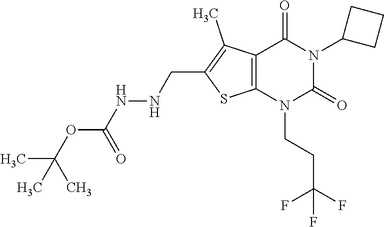 [SMILES: Cc1c(CNNC(=O)OC(C)(C)C)sc2c1c(=O)n(C1CCC1)c(=O)n2CCC(F)(F)F]

To a solution of 450 mg (0.948 mmol) of the compound from Ex. 184A in 9.5 ml of methanol were added 298 mg (4.74 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h and after 3 h, a further 149 mg (2.37 mmol) of sodium cyanoborohydride were added in each case. Over the entire reaction time, by addition of further acetic acid, the pH was constantly regulated such that the indicator colour just remained yellow. After a total of 7 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was stirred with acetonitrile at RT. After the product had been filtered off with suction and dried under high vacuum, 402 mg (85% of theory; 96% purity) of the title compound were obtained.

¹H-NMR (400 MHz, DMSO-d₆, δ/ppm): 8.25 (br. s, 1H), 5.22 (quin, 1H), 5.05 (br. d, 1H), 4.09 (t, 2H), 3.98 (d, 2H), 2.91-2.69 (m, 4H), 2.31 (s, 3H), 2.22-2.10 (m, 2H), 1.87-1.64 (m, 2H), 1.38 (s, 9H).

LC/MS (Method 1, ESIneg): Rₜ=2.22 min, m/z=475.16 [M−H]⁻.

### Example 229A

### tert-Butyl 2-{[3-cyclobutyl-1-(2-methoxyethyl)-5-methyl-2,4-dioxo-1,2,3,4-tetrahydrothieno[2,3-d]pyrimidin-6-yl]methyl}hydrazinecarboxylate

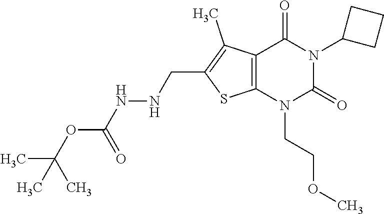 [SMILES: COCCn1c(=O)n(C2CCC2)c(=O)c2c(C)c(CNNC(=O)OC(C)(C)C)sc21]

To a solution of 350 mg (0.802 mmol) of the compound from Ex. 185A in 8 ml of methanol were added 252 mg (4.01 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h and after 3 h, a further 126 mg (2.00 mmol) of sodium cyanoborohydride were added in each case. Over the entire reaction time, by addition of further acetic acid, the pH was constantly regulated such that the indicator colour just remained yellow. After a total of 7 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was stirred with acetonitrile at RT. After the product had been filtered off with suction and dried under high vacuum, 266 mg (75% of theory) of the title compound were obtained.

¹H-NMR (400 MHz, DMSO-d₆, δ/ppm): 8.25 (br. s, 1H), 5.23 (quin, 1H), 4.97 (br. d, 1H), 4.00 (t, 2H), 3.96 (br. d, 2H), 3.64 (t, 2H), 3.25 (s, 3H), 2.91-2.78 (m, 2H), 2.31 (s, 3H), 2.16 (dtd, 2H), 1.87-1.63 (m, 2H), 1.39 (s, 9H).

LC/MS (Method 1, ESIneg): Rₜ=1.99 min, m/z=483.19 [M−H+HCOOH]⁻.

### Example 230A

### tert-Butyl 2-{[3-(3,3-difluorocyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

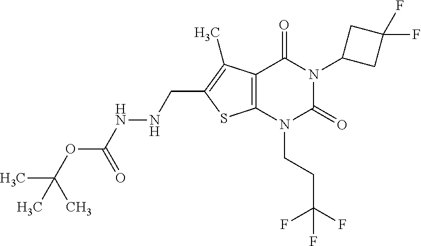 [SMILES: Cc1c(CNNC(=O)OC(C)(C)C)sc2c1c(=O)n(C1CC(F)(F)C1)c(=O)n2CCC(F)(F)F]

To a solution of 670 mg (1.22 mmol, 93% purity) of the compound from Ex. 186A in 12 ml of methanol were added 384 mg (6.11 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 2 h and after 4 h, a further 192 mg (2.50 mmol) of sodium cyanoborohydride were added in each case.

After a total of 16 h at 65° C., the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 630 mg (83% of theory, 82% purity) of the title compound were obtained, which was used for subsequent reactions without further purification.

LC/MS (Method 2, ESIpos): Rₜ=1.18 min, m/z=405 [M+H]⁺.

### Example 231A

### tert-Butyl 2-{[3-(3,3-difluorocyclobutyl)-1-(2-methoxethyl)-5-methyl-2,4-dioxo-1,2,3,4-tetrahydrothieno[2,3-d]pyrimidin-6-yl]methyl}hydrazinecarboxylate

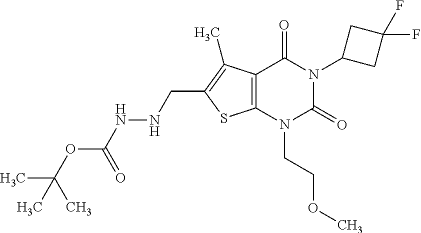 [SMILES: COCCn1c(=O)n(C2CC(F)(F)C2)c(=O)c2c(C)c(CNNC(=O)OC(C)(C)C)sc21]

To a solution of 697 mg (1.27 mmol, 86% purity) of the compound from Ex. 187A in 12 ml of methanol were added 400 mg (6.37 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 2 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by preparative HPLC (Method 13). After combination of the product fractions, concentration and drying under high vacuum, 194 mg (32% of theory) of the title compound were obtained.

LC/MS (Method 2, ESIneg): Rₜ=1.07 min, m/z=473 [M−H]⁻.

### Example 232A

### tert-Butyl 2-{[5-methyl-3-(1-methylcyclobutyl)-2,4-dioxo-1-(3,3,3-trifluoropropyl)-1,2,3,4-tetrahydrothieno[2,3-d]pyrimidin-6-yl]methyl}hydrazinecarboxylate

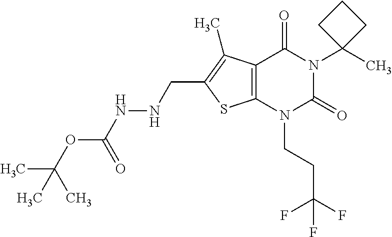 [SMILES: Cc1c(CNNC(=O)OC(C)(C)C)sc2c1c(=O)n(C1(C)CCC1)c(=O)n2CCC(F)(F)F]

Analogously to the method described in Ex. 200A, 345 mg (0.706 mmol) of the compound from Ex. 188A and a total of 333 mg (5.30 mmol) of sodium cyanoborohydride were used to prepare 330 mg (95% of theory) of the title compound.

¹H-NMR (400 MHz, DMSO-d₆, δ/ppm): 8.23 (br. s, 1H), 5.03 (br. d, 1H), 4.06 (br. t, 2H), 3.97 (br. d, 2H), 2.82-2.65 (m, 2H), 2.35-2.23 (m, 4H), 2.29 (s, 3H), 1.81-1.57 (m, 2H), 1.51 (s, 3H), 1.38 (s, 9H).

LC/MS (Method 2, ESIneg): Rₜ=1.21 min, m/z=489 [M−H]⁻.

### Example 233A

### tert-Butyl 2-{[1-(2-methoxyethyl)-5-methyl-3-(1-methylcyclobutyl)-2,4-dioxo-1,2,3,4-tetrahydrothieno[2,3-d]pyrimidin-6-yl]methyl}hydrazinecarboxylate

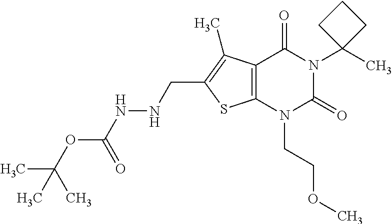 [SMILES: COCCn1c(=O)n(C2(C)CCC2)c(=O)c2c(C)c(CNNC(=O)OC(C)(C)C)sc21]

Analogously to the method described in Ex. 200A, 400 mg (0.888 mmol) of the compound from Ex. 189A and a total of 418 mg (6.66 mmol) of sodium cyanoborohydride were used to prepare 305 mg (75% of theory) of the title compound.

¹H-NMR (400 MHz, DMSO-d₆, δ/ppm): 8.23 (br. s, 1H), 4.95 (br. s, 1H), 4.00-3.91 (m, 4H), 3.61 (t, 2H), 3.25 (s, 3H), 2.36-2.21 (m, 4H), 2.28 (s, 3H), 1.79-1.57 (m, 2H), 1.51 (s, 3H), 1.39 (s, 9H).

### Example 234A

### tert-Butyl 2-{[3-(trans-3-methoxycyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

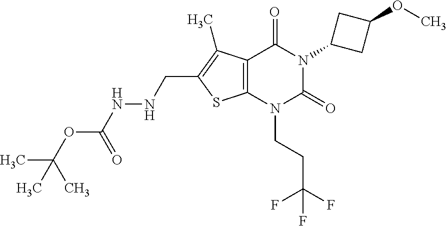 [SMILES: CO[C@H]1C[C@H](n2c(=O)c3c(C)c(CNNC(=O)OC(C)(C)C)sc3n(CCC(F)(F)F)c2=O)C1]

To a solution of 946 mg (1.53 mmol, 82% purity) of the compound from Ex. 190A in 18 ml of methanol were added 482 mg (7.67 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 24 h, another 240 mg of sodium cyanoborohydride were added and stirring of the mixture was continued at RT for 18 h. The volatile constituents of the reaction mixture was then substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 1.11 g (90% of theory, 63% purity) of the title compound was obtained, which was used for subsequent reactions without further purification.

LC/MS (Method 2, ESIneg): Rₜ=1.08 min, m/z=551 [M−H+HCOOH]⁻.

### Example 235A

### tert-Butyl 2-{[3-(trans-3-methoxycyclobutyl)-1-(2-methoxyethyl)-5-methyl-2,4-dioxo-1,2,3,4-tetrahydrothieno[2,3-d]pyrimidin-6-yl]methyl}hydrazinecarboxylate

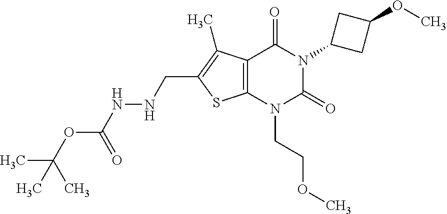 [SMILES: COCCn1c(=O)n([C@H]2C[C@H](OC)C2)c(=O)c2c(C)c(CNNC(=O)OC(C)(C)C)sc21]

To a solution of 1.07 g (1.64 mmol, 71% purity) of the compound from Ex. 191A in 22 ml of methanol were added 514 mg (8.18 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 18 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 1.02 g (98% of theory, 74% purity) of the title compound was obtained, which was used for subsequent reactions without further purification.

LC/MS (Method 2, ESIneg): Rₜ=0.94 min, m/z=513 [M−H+HCOOH]⁻.

### Example 236A

### tert-Butyl 2-{[3-(cis-3-methoxycyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

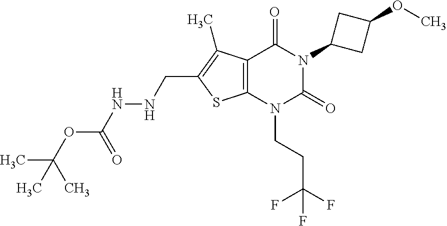 [SMILES: CO[C@H]1C[C@@H](n2c(=O)c3c(C)c(CNNC(=O)OC(C)(C)C)sc3n(CCC(F)(F)F)c2=O)C1]

To a solution of 0.53 g (1.05 mmol) of the compound from Ex. 192A in 14 ml of methanol were added 330 mg (5.25 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 20 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 0.51 g (73% of theory, 76% purity) of the title compound were obtained, which was used for subsequent reactions without further purification.

LC/MS (Method 1, ESIneg): Rₜ=2.02 min, m/z=505 [M−H+HCOOH]⁻.

### Example 237A

### tert-Butyl 2-{[3-(cis-3-methoxycyclobutyl)-1-(2-methoxyethyl)-5-methyl-2,4-dioxo-1,2,3,4-tetrahydrothieno[2,3-d]pyrimidin-6-yl]methyl}hydrazinecarboxylate

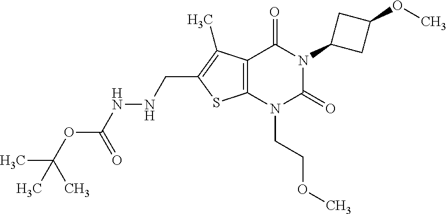 [SMILES: COCCn1c(=O)n([C@H]2C[C@@H](OC)C2)c(=O)c2c(C)c(CNNC(=O)OC(C)(C)C)sc21]

To a solution of 0.84 g (1.45 mmol, 80% purity) of the compound from Ex. 193A in 20 ml of methanol were added 454 mg (7.23 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 20 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 0.48 g (71% of theory, 60% purity) of the title compound was obtained, which was used for subsequent reactions without further purification.

LC/MS (Method 1, ESIneg): Rₜ=1.75 min, m/z=513 [M−H+HCOOH]⁻.

### Example 238A

### tert-Butyl 2-{[3-(3,3-dimethylcyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

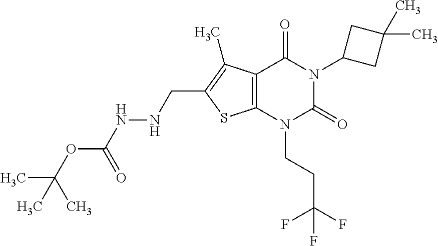 [SMILES: Cc1c(CNNC(=O)OC(C)(C)C)sc2c1c(=O)n(C1CC(C)(C)C1)c(=O)n2CCC(F)(F)F]

To a solution of 425 mg (0.85 mmol, 87% purity) of the compound from Ex. 194A in 11 ml of methanol were added 266 mg (4.23 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 24 h, the reaction mixture was concentrated. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 0.40 g (92% of theory, 83% purity) of the title compound was obtained, which was used for subsequent reactions without further purification.

LC/MS (Method 1, ESIneg): Rₜ=2.46 min, m/z=549 [M−H+HCOOH]⁻.

### Example 239A

### tert-Butyl 2-{[3-(3,3-dimethylcyclobutyl)-1-(2-methoxyethyl)-5-methyl-2,4-dioxo-1,2,3,4-tetrahydrothieno[2,3-d]pyrimidin-6-yl]methyl}hydrazinecarboxylate

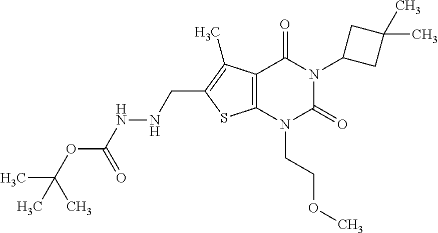 [SMILES: COCCn1c(=O)n(C2CC(C)(C)C2)c(=O)c2c(C)c(CNNC(=O)OC(C)(C)C)sc21]

To a solution of 745 mg (1.30 mmol, 81% purity) of the compound from Ex. 195A in 17 ml of methanol were added 408 mg (6.49 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After a total of 24 h, the reaction mixture was admixed with saturated aqueous sodium hydrogencarbonate solution and concentrated. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. 0.59 g (98% of theory, 56% purity) of the title compound was obtained, which was used for subsequent reactions without further purification.

LC/MS (Method 1, ESIneg): Rₜ=2.28 min, m/z=511 [M−H+HCOOH]⁻.

### Example 240A

### tert-Butyl 2-{[5-methyl-2,4-dioxo-3-(spiro[3.3]hept-2-yl)-1-(3,3,3-trifluoropropyl)-1,2,3,4-tetrahydrothieno[2,3-d]pyrimidin-6-yl]methyl}hydrazinecarboxylate

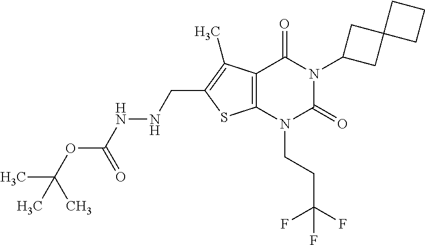 [SMILES: Cc1c(CNNC(=O)OC(C)(C)C)sc2c1c(=O)n(C1CC3(CCC3)C1)c(=O)n2CCC(F)(F)F]

Analogously to the method described in Ex. 200A, 485 mg (0.943 mmol) of the compound from Ex. 196A and a total of 444 mg (7.07 mmol) of sodium cyanoborohydride were used to prepare 437 mg (86% of theory, 96% of theory) of the title compound.

¹H-NMR (400 MHz, DMSO-d₆, δ/ppm): 8.24 (br. s, 1H), 5.11-4.96 (m, 2H), 4.07 (br. t, 2H), 3.97 (br. d, 2H), 2.89-2.62 (m, 4H), 2.30 (s, 3H), 2.23 (td, 2H), 2.10-2.03 (m, 2H), 2.01-1.93 (m, 2H), 1.86-1.74 (m, 2H), 1.38 (s, 9H).

LC/MS (Method 2, ESIneg): Rₜ=1.33 min, m/z=561 [M−H+HCOOH]⁻.

### Example 241A

### tert-Butyl 2-{[1-(2-methoxyethyl)-5-methyl-2,4-dioxo-3-(spiro[3.3]hept-2-yl)-1,2,3,4-tetrahydrothieno[2,3-d]pyrimidin-6-yl]methyl}hydrazinecarboxylate

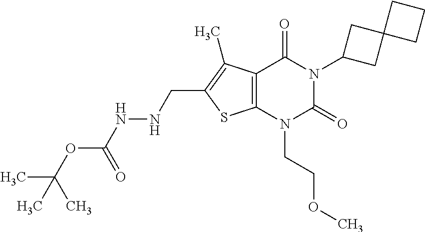 [SMILES: COCCn1c(=O)n(C2CC3(CCC3)C2)c(=O)c2c(C)c(CNNC(=O)OC(C)(C)C)sc21]

Analogously to the method described in Ex. 200A, 500 mg (1.05 mmol) of the compound from Ex. 197A and a total of 494 mg (7.87 mmol) of sodium cyanoborohydride were used to prepare 392 mg (78% of theory) of the title compound.

¹H-NMR (400 MHz, DMSO-d₆, δ/ppm): 8.23 (br. s, 1H), 5.07 (quin, 1H), 4.96 (br. s, 1H), 3.99 (t, 2H), 3.95 (br. d, 2H), 3.62 (t, 2H), 3.25 (s, 3H), 2.91-2.78 (m, 2H), 2.30 (s, 3H), 2.23 (td, 2H), 2.11-2.03 (m, 2H), 2.01-1.94 (m, 2H), 1.86-1.74 (m, 2H), 1.38 (s, 9H).

LC/MS (Method 2, ESIneg): Rₜ=1.24 min, m/z=523 [M−H+HCOOH]⁻.

### Example 242A

### tert-Butyl 2-{[3-cyclopentyl-5-methyl-2,4-dioxo-1-(3,3,3-trifluoropropyl)-1,2,3,4-tetrahydrothieno[2,3-d]pyrimidin-6-yl]methyl}hydrazinecarboxylate

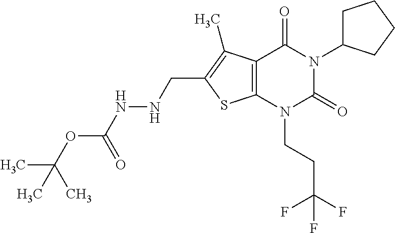 [SMILES: Cc1c(CNNC(=O)OC(C)(C)C)sc2c1c(=O)n(C1CCCC1)c(=O)n2CCC(F)(F)F]

Analogously to the method described in Ex. 200A, 477 mg (0.976 mmol) of the compound from Ex. 198A and a total of 460 mg (7.32 mmol) of sodium cyanoborohydride were used to prepare 435 mg (80% of theory, 88% purity) of the title compound, which was used for subsequent reactions without further purification.

¹H-NMR (400 MHz, DMSO-d₆, δ/ppm): 8.24 (br. s, 1H), 5.30 (quin, 1H), 5.05 (br. d, 1H), 4.10 (t, 2H), 3.98 (br. d, 2H), 2.86-2.69 (m, 2H), 2.32 (s, 3H), 2.09-1.97 (m, 2H), 1.93-1.84 (m, 2H), 1.80-1.66 (m, 2H), 1.62-1.48 (m, 2H), 1.38 (s, 9H).

LC/MS (Method 2, ESIneg): Rₜ=1.23 min, m/z=489 [M−H]⁻.

### Example 243A

### tert-Butyl 2-{[3-cyclopentyl-1-(2-methoxyethyl)-5-methyl-2,4-dioxo-1,2,3,4-tetrahydrothieno[2,3-d]pyrimidin-6-yl]methyl}hydrazinecarboxylate

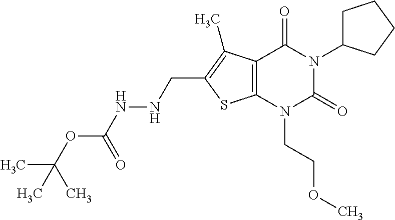 [SMILES: COCCn1c(=O)n(C2CCCC2)c(=O)c2c(C)c(CNNC(=O)OC(C)(C)C)sc21]

Analogously to the method described in Ex. 200A, 505 mg (1.05 mmol, 94% purity) of the compound from Ex. 199A and a total of 497 mg (7.90 mmol) of sodium cyanoborohydride were used to prepare 494 mg (91% of theory, 88% purity) of the title compound. Purification of the product by means of MPLC was dispensed with here.

¹H-NMR (400 MHz, DMSO-d₆, δ/ppm): 8.24 (br. s, 1H), 5.30 (quin, 1H), 4.98 (br. s, 1H), 4.01 (t, 2H), 3.96 (br. d, 2H), 3.64 (t, 2H), 3.25 (s, 3H), 2.32 (s, 3H), 2.08-1.96 (m, 2H), 1.95-1.82 (m, 2H), 1.80-1.68 (m, 2H), 1.62-1.48 (m, 2H), 1.39 (s, 9H).

LC/MS (Method 2, ESIneg): Rₜ=1.11 min, m/z=497 [M−H+HCOOH]⁻.

### Example 244A

### tert-Butyl 2-carbamoyl-2-[(3-cyclopropyl-1,5-dimethyl-2,4-dioxo-1,2,3,4-tetrahydrothieno[2,3-d]pyrimidin-6-yl)methyl]hydrazinecarboxylate

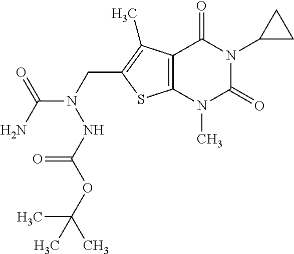 [SMILES: Cc1c(CN(NC(=O)OC(C)(C)C)C(N)=O)sc2c1c(=O)n(C1CC1)c(=O)n2C]

To a solution of 230 mg (0.605 mmol) of the compound from Ex. 200A in 15 ml of isopropanol were added 162 μl (1.21 mmol) of trimethylsilyl isocyanate, and the mixture was stirred at RT for 2.5 days. The reaction mixture was then concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and concentrated. The residue was purified by means of MPLC (Biotage Isolera One, SNAP KP-Sil cartridge, 50 g of silica gel, eluent: cyclohexane/ethyl acetate 1:2). Concentration of the product fractions and drying of the residue under high vacuum gave 130 mg (50% of theory) of the title compound.

¹H-NMR (400 MHz, DMSO-d₆, δ/ppm): 8.91 (br. s, 1H), 6.16 (br. s, 2H), 5.17-3.93 (broad, 2H), 3.38 (s, 3H), 2.64-2.56 (m, 1H), 2.32 (s, 3H), 1.38 (br. s, 9H), 1.07-0.95 (m, 2H), 0.68-0.62 (m, 2H).

LC/MS (Method 2, ESIpos): Rₜ=0.73 min, m/z=424 [M+H]⁺.

### Example 245A

### tert-Butyl 2-[(1-butyl-3-cyclopropyl-5-methyl-2,4-dioxo-1,2,3,4-tetrahy drothieno[2,3-d]pyrimidin-6-yl)methyl]-2-carbamoylhydrazinecarboxylate

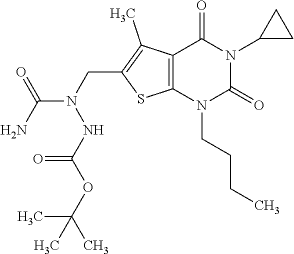 [SMILES: CCCCn1c(=O)n(C2CC2)c(=O)c2c(C)c(CN(NC(=O)OC(C)(C)C)C(N)=O)sc21]

Analogously to the method described in Ex. 244A, 316 mg (0.688 mmol, 92% purity) of the compound from Ex. 201A and 185 μl (1.38 mmol) of trimethylsilyl isocyanate were used to prepare 375 mg (99% of theory, 85% purity) of the title compound. Purification of the product by means of MPLC was dispensed with here.

LC/MS (Method 1, ESIneg): Rₜ=1.62 min, m/z=464.20 [M−H]⁻.

### Example 246A

### tert-Butyl 2-carbamoyl-2-{[3-cyclopropyl-5-methyl-2,4-dioxo-1-(3,3,3-trifluoropropyl)-1,2,3,4-tetrahydrothieno[2,3-d]pyrimidin-6-yl]methyl}hydrazinecarboxylate

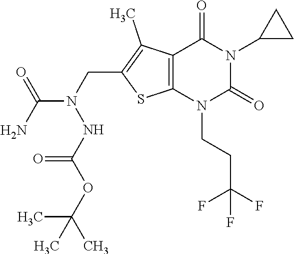 [SMILES: Cc1c(CN(NC(=O)OC(C)(C)C)C(N)=O)sc2c1c(=O)n(C1CC1)c(=O)n2CCC(F)(F)F]

To a solution of 150 mg (0.324 mmol) of the compound from Ex. 202A in 10 ml of isopropanol were added 130 μl (0.973 mmol) of trimethylsilyl isocyanate, and the mixture was stirred at 50° C. for 5 h. The reaction mixture was then concentrated to dryness. The residue that remained was purified directly by means of MPLC (Biotage Isolera One, SNAP KP-Sil cartridge, 10 g of silica gel, eluent: cyclohexane/ethyl acetate 1:1→dichloromethane/methanol 20:1). Concentration of the product fractions and drying under high vacuum gave 181 mg (92% of theory, 84% purity) of the title compound, which was used for subsequent reactions without further purification.

¹H-NMR (400 MHz, DMSO-d₆, δ/ppm): 8.91 (br. s, 1H), 6.19 (s, 2H), 5.12-4.18 (broad, 2H), 4.08 (br. t, 2H), 2.80-2.65 (m, 2H), 2.61 (tt, 1H), 2.33 (s, 3H), 1.38 (br. s, 9H), 1.07-0.97 (m, 2H), 0.71-0.57 (m, 2H).

LC/MS (Method 1, ESIneg): Rₜ=1.58 min, m/z=504.15 [M−H]⁻.

### Example 247A

### tert-Butyl 2-carbamoyl-2-{[1-(cyclobutylmethyl)-3-cyclopropyl-5-methyl-2,4-dioxo-1,2,3,4-tetrahydrothieno[2,3-d]pyrimidin-6-yl]methyl}hydrazinecarboxylate

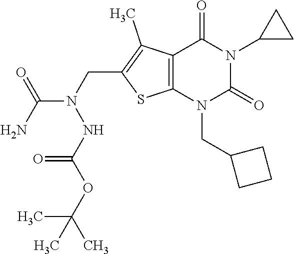 [SMILES: Cc1c(CN(NC(=O)OC(C)(C)C)C(N)=O)sc2c1c(=O)n(C1CC1)c(=O)n2CC1CCC1]

Analogously to the method described in Ex. 244A, 375 mg (0.781 mmol, 90% purity) of the compound from Ex. 203A and 209 μl (1.56 mmol) of trimethylsilyl isocyanate were used to prepare 372 mg (99% of theory) of the title compound. The reaction time here was about 16 h.

LC/MS (Method 1, ESIneg): Rₜ=1.68 min, m/z=476.20 [M−H]⁻.

### Example 248A

### tert-Butyl 2-carbamoyl-2-({3-cyclopropyl-1-[(2,2-difluorocyclopropyl)methyl]-5-methyl-2,4-dioxo-1,2,3,4-tetrahydrothieno[2,3-d]pyrimidin-6-yl}methyl)hydrazinecarboxylate (racemate)

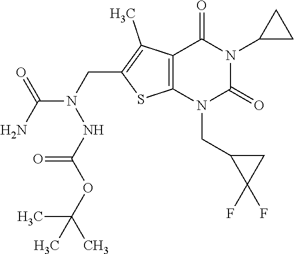 [SMILES: Cc1c(CN(NC(=O)OC(C)(C)C)C(N)=O)sc2c1c(=O)n(C1CC1)c(=O)n2CC1CC1(F)F]

Analogously to the method described in Ex. 244A, 685 mg (1.35 mmol, 90% purity) of the compound from Ex. 204A and 362 μl (2.70 mmol) of trimethylsilyl isocyanate were used to prepare 660 mg (90% of theory, 93% purity) of the title compound. The reaction time in this case was 7 days.

¹H-NMR (400 MHz, DMSO-d₆, δ/ppm): 8.91 (br. s, 1H), 6.18 (br. s, 2H), 5.03-4.13 (broad, 2H), 4.12-3.88 (m, 2H), 2.66-2.58 (m, 1H), 2.33 (s, 3H), 2.18 (td, 1H), 1.76-1.63 (m, 1H), 1.52-1.43 (m, 1H), 1.38 (br. s, 9H), 1.08-0.94 (m, 2H), 0.68-0.63 (m, 2H).

LC/MS (Method 1, ESIneg): Rₜ=1.54 min, m/z=498.16 [M−H]⁻.

### Example 249A

### tert-Butyl 2-carbamoyl-2-{[1-(cyanomethyl)-3-cyclopropyl-5-methyl-2,4-dioxo-1,2,3,4-tetrahydrothieno[2,3-d]pyrimidin-6-yl]methyl}hydrazinecarboxylate

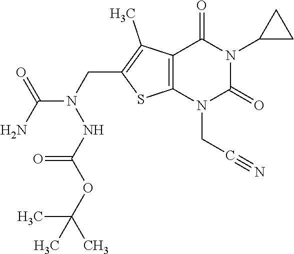 [SMILES: Cc1c(CN(NC(=O)OC(C)(C)C)C(N)=O)sc2c1c(=O)n(C1CC1)c(=O)n2CC#N]

Analogously to the method described in Ex. 244A, 220 mg (0.543 mmol) of the compound from Ex. 205A and 146 μl (1.09 mmol) of trimethylsilyl isocyanate were used to prepare 230 mg (89% of theory, 95% purity) of the title compound. The reaction time here was about 16 h.

LC/MS (Method 1, ESIneg): Rₜ=1.31 min, m/z=447.15 [M−H]⁻.

### Example 250A

### tert-Butyl 2-carbamoyl-2-{[3-cyclopropyl-1-(2-methoxyethyl)-5-methyl-2,4-dioxo-1,2,3,4-tetrahydrothieno[2,3-d]pyrimidin-6-yl]methyl}hydrazinecarboxylate

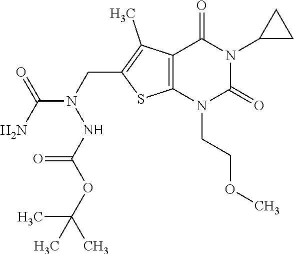 [SMILES: COCCn1c(=O)n(C2CC2)c(=O)c2c(C)c(CN(NC(=O)OC(C)(C)C)C(N)=O)sc21]

To a solution of 365 mg (0.860 mmol) of the compound from Ex. 206A in 30 ml of isopropanol were added 353 μl (2.58 mmol) of trimethylsilyl isocyanate, and the mixture was stirred at 50° C. for about 16 h. Thereafter, the reaction mixture was concentrated to about one third of the original volume. In the course of this, a portion of the product precipitated out, which was filtered off with suction. The mother liquor was concentrated further and then separated into its components by means of preparative HPLC (Method 11). The product fractions were concentrated, combined with the solids previously removed, and dried under high vacuum. 259 mg (59% of theory, 93% purity) of the title compound were obtained, which was used for subsequent reactions without further purification.

¹H-NMR (400 MHz, DMSO-d₆, δ/ppm): 8.91 (br. s, 1H), 6.17 (br. s, 2H), 5.19-4.10 (broad, 2H), 3.99 (t, 2H), 3.62 (t, 2H), 3.24 (s, 3H), 2.61 (tt, 1H), 2.31 (s, 3H), 1.38 (br. s, 9H), 1.07-0.94 (m, 2H), 0.70-0.58 (m, 2H).

LC/MS (Method 1, ESIneg): Rₜ=1.31 min, m/z=466.18 [M−H]⁻.

### Example 251A

### tert-Butyl 2-carbamoyl-2-{[3-cyclopropyl-1-(2-ethoxyethyl)-5-methyl-2,4-dioxo-1,2,3,4-tetrahydrothieno[2,3-d]pyrimidin-6-yl]methyl}hydrazinecarboxylate

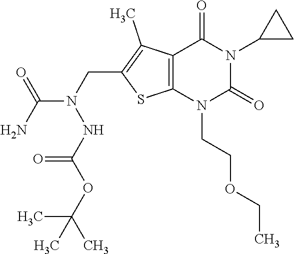 [SMILES: CCOCCn1c(=O)n(C2CC2)c(=O)c2c(C)c(CN(NC(=O)OC(C)(C)C)C(N)=O)sc21]

Analogously to the method described in Ex. 244A, 230 mg (0.524 mmol) of the compound from Ex. 207A and 141 μl (1.05 mmol) of trimethylsilyl isocyanate were used to prepare 262 mg (98% of theory, 95% purity) of the title compound. The reaction time here was about 16 h.

LC/MS (Method 2, ESIneg): Rₜ=0.78 min, m/z=480 [M−H]⁻.

### Example 252A

### tert-Butyl 2-carbamoyl-2-{[3-cyclopropyl-1-(2-isopropoxyethyl)-5-methyl-2,4-dioxo-1,2,3,4-tetrahydrothieno[2,3-d]pyrimidin-6-yl]methyl}hydrazinecarboxylate

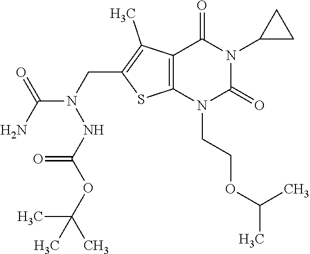 [SMILES: Cc1c(CN(NC(=O)OC(C)(C)C)C(N)=O)sc2c1c(=O)n(C1CC1)c(=O)n2CCOC(C)C]

Analogously to the method described in Ex. 244A, 375 mg (0.829 mmol) of the compound from Ex. 208A and 222 μl (1.66 mmol) of trimethylsilyl isocyanate were used to prepare 410 mg (99% of theory) of the title compound. The reaction time here was about 16 h.

LC/MS (Method 1, ESIneg): Rₜ=1.56 min, m/z=494.21 [M−H]⁻.

### Example 253A

### tert-Butyl 2-carbamoyl-2-({3-cyclopropyl-5-methyl-2,4-dioxo-1-[2-(trifluoromethoxy)ethyl]-1,2,3,4-tetrahydrothieno[2,3-d]pyrimidin-6-yl}methyl)hydrazinecarboxylate

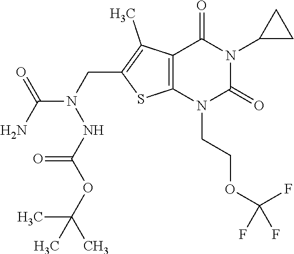 [SMILES: Cc1c(CN(NC(=O)OC(C)(C)C)C(N)=O)sc2c1c(=O)n(C1CC1)c(=O)n2CCOC(F)(F)F]

To a solution of 335 mg (0.665 mmol, 95% purity) of the compound from Ex. 209A in 15 ml of isopropanol were added 178 μl (1.33 mmol) of trimethylsilyl isocyanate, and the mixture was stirred at RT for 2.5 days. The reaction mixture was then concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and concentrated. After drying under high vacuum, 360 mg (98% of theory, 95% purity) of the title compound were obtained.

LC/MS (Method 1, ESIpos): Rₜ=1.60 min, m/z=520.15 [M+H]⁺.

### Example 254A

### tert-Butyl 2-carbamoyl-2-{[3-cyclopropyl-5-methyl-2,4-dioxo-1-(tetrahydrofuran-2-ylmethyl)-1,2,3,4-tetrahydrothieno[2,3-d]pyrimidin-6-yl]methyl}hydrazinecarboxylate (racemate)

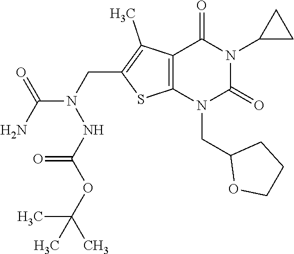 [SMILES: Cc1c(CN(NC(=O)OC(C)(C)C)C(N)=O)sc2c1c(=O)n(C1CC1)c(=O)n2CC1CCCO1]

Analogously to the method described in Ex. 250A, 365 mg (0.810 mmol) of the compound from Ex. 210A and 333 μl (2.43 mmol) of trimethylsilyl isocyanate were used to prepare 358 mg (85% of theory, 95% purity) of the title compound.

¹H-NMR (400 MHz, DMSO-d₆, δ/ppm): 8.94 (br. s, 1H), 6.17 (s, 2H), 5.14-4.29 (broad, 2H), 4.29-4.16 (m, 1H), 3.96 (dd, 1H), 3.81-3.66 (m, 2H), 3.61 (q, 1H), 2.65-2.57 (m, 1H), 2.31 (s, 3H), 2.02-1.75 (m, 3H), 1.72-1.59 (m, 1H), 1.38 (br. s, 9H), 1.09-0.94 (m, 2H), 0.71-0.55 (m, 2H).

LC/MS (Method 1, ESIneg): Rₜ=1.40 min, m/z=492.19 [M−H]⁻.

### Example 255A

### tert-Butyl 2-carbamoyl-2-({3-cyclopropyl-5-methyl-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

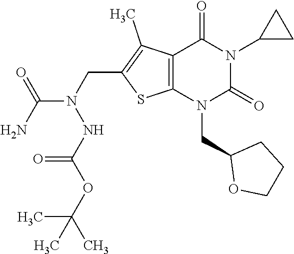 [SMILES: Cc1c(CN(NC(=O)OC(C)(C)C)C(N)=O)sc2c1c(=O)n(C1CC1)c(=O)n2C[C@H]1CCCO1]

Analogously to the method described in Ex. 244A, 220 mg (0.488 mmol) of the compound from Ex. 211A and 131 μl (0.977 mmol) of trimethylsilyl isocyanate were used to prepare 260 mg (97% of theory, 90% purity) of the title compound. The reaction time here was about 16 h.

LC/MS (Method 1, ESIneg): Rₜ=1.42 min, m/z=492.19 [M−H]⁻.

### Example 256A

### tert-Butyl 2-carbamoyl-2-({3-cyclopropyl-5-methyl-2,4-dioxo-1-[(2S)-tetrahydrofuran-2-ylmethyl]-1,2,3,4-tetrahydrothieno[2,3-d]pyrimidin-6-yl}methyl)hydrazinecarboxylate

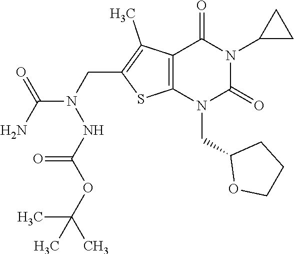 [SMILES: Cc1c(CN(NC(=O)OC(C)(C)C)C(N)=O)sc2c1c(=O)n(C1CC1)c(=O)n2C[C@@H]1CCCO1]

Analogously to the method described in Ex. 244A, 175 mg (0.388 mmol) of the compound from Ex. 212A and 104 μl (0.777 mmol) of trimethylsilyl isocyanate were used to prepare 212 mg (99% of theory, 90% purity) of the title compound. The reaction time here was about 16 h.

LC/MS (Method 1, ESIneg): Rₜ=1.40 min, m/z=492.19 [M−H]⁻.

### Example 257A

### tert-Butyl 2-carbamoyl-2-{[1,5-dimethyl-3-(1-methylcyclopropyl)-2,4-dioxo-1,2,3,4-tetrahydrothieno[2,3-d]pyrimidin-6-yl]methyl}hydrazinecarboxylate

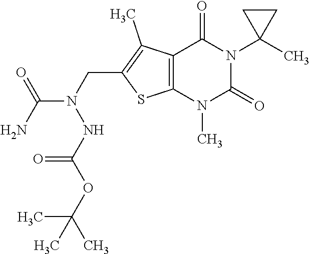 [SMILES: Cc1c(CN(NC(=O)OC(C)(C)C)C(N)=O)sc2c1c(=O)n(C1(C)CC1)c(=O)n2C]

Analogously to the method described in Ex. 244A, 265 mg (0.638 mmol, 95% purity) of the compound from Ex. 213A and 171 μl (1.28 mmol) of trimethylsilyl isocyanate were used to prepare 280 mg (95% of theory, 95% purity) of the title compound. The reaction time here was about 16 h.

¹H-NMR (400 MHz, DMSO-d₆, δ/ppm): 8.89 (br. s, 1H), 6.16 (br. s, 2H), 5.13-4.10 (broad, 2H), 3.38 (s, 3H), 2.33 (s, 3H), 1.38 (br. s, 9H), 1.33 (s, 3H), 0.97-0.74 (m, 4H).

LC/MS (Method 1, ESIneg): Rₜ=1.40 min, m/z=436.17 [M−H]⁻.

### Example 258A

### tert-Butyl 2-{[1-butyl-5-methyl-3-(1-methylcyclopropyl)-2,4-dioxo-1,2,3,4-tetrahydrothieno[2,3-d]pyrimidin-6-yl]methyl}-2-carbamoylhydrazinecarboxylate

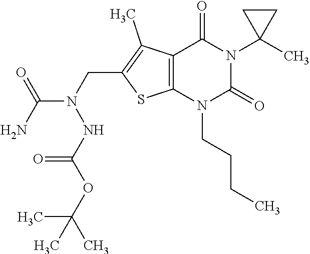 [SMILES: CCCCn1c(=O)n(C2(C)CC2)c(=O)c2c(C)c(CN(NC(=O)OC(C)(C)C)C(N)=O)sc21]

Analogously to the method described in Ex. 244A, 350 mg (0.802 mmol) of the compound from Ex. 214A and 215 μl (1.60 mmol) of trimethylsilyl isocyanate were used to prepare 322 mg (74% of theory, 89% purity) of the title compound. The reaction time here was about 16 h.

LC/MS (Method 1, ESIneg): Rₜ=1.77 min, m/z=478.21 [M−H]⁻.

### Example 259A

### tert-Butyl 2-carbamoyl-2-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methyl}hydrazinecarboxylate

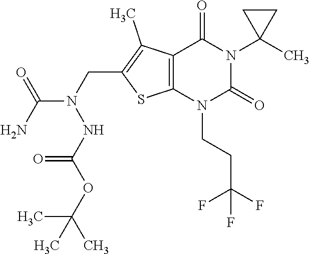 [SMILES: Cc1c(CN(NC(=O)OC(C)(C)C)C(N)=O)sc2c1c(=O)n(C1(C)CC1)c(=O)n2CCC(F)(F)F]

To a solution of 1.28 g (2.11 mmol, 79% purity) of the compound from Ex. 215A in 27 ml of isopropanol were added 487 mg (4.22 mmol) of trimethylsilyl isocyanate. After a total of 24 h at RT, the volatile constituents of the reaction mixture were substantially removed on a rotary evaporator. 1.55 g (100% of theory, 74% purity) of the title compound were obtained, which was used for subsequent reactions without further purification.

LC/MS (Method 2, ESIpos): Rₜ=0.89 min, m/z=520 [M+H]⁺.

### Example 260A

### tert-Butyl 2-carbamoyl-2-{[1-(cyclobutylmethyl)-5-methyl-3-(1-methylcyclopropyl)-2,4-dioxo-1,2,3,4-tetrahydrothieno[2,3-d]pyrimidin-6-yl]methyl}hydrazinecarboxylate

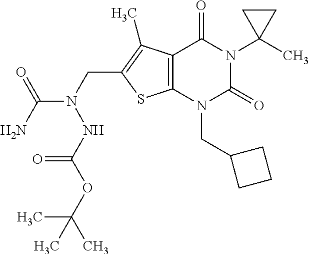 [SMILES: Cc1c(CN(NC(=O)OC(C)(C)C)C(N)=O)sc2c1c(=O)n(C1(C)CC1)c(=O)n2CC1CCC1]

Analogously to the method described in Ex. 244A, 245 mg (0.546 mmol) of the compound from Ex. 216A and 147 μl (1.09 mmol) of trimethylsilyl isocyanate were used to prepare 255 mg (85% of theory, 90% purity) of the title compound. The reaction time here was about 16 h.

¹H-NMR (400 MHz, DMSO-d₆, δ/ppm): 8.88 (br. s, 1H), 6.18 (br. s, 2H), 4.95-4.13 (broad, 2H), 4.07-3.75 (m, 2H), 2.83-2.69 (m, 1H), 2.31 (s, 3H), 2.04-1.89 (m, 2H), 1.87-1.74 (m, 4H), 1.38 (br. s, 9H), 1.32 (s, 3H), 0.99-0.70 (m, 4H).

LC/MS (Method 2, ESIneg): Rₜ=0.96 min, m/z=490 [M−H]⁻.

### Example 261A

### tert-Butyl 2-carbamoyl-2-({1-[(2,2-difluorocyclopropyl)methyl]-5-methyl-3-(1-methylcyclopropyl)-2,4-dioxo-1,2,3,4-tetrahydrothieno[2,3-d]pyrimidin-6-yl}methyl)hydrazinecarboxylate (racemate)

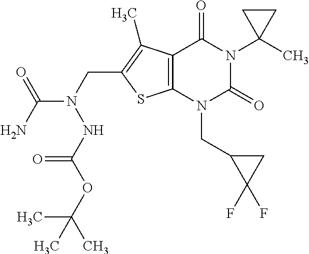 [SMILES: Cc1c(CN(NC(=O)OC(C)(C)C)C(N)=O)sc2c1c(=O)n(C1(C)CC1)c(=O)n2CC1CC1(F)F]

Analogously to the method described in Ex. 244A, 680 mg (1.32 mmol, 91% purity) of the compound from Ex. 217A and 353 μl (2.63 mmol) of trimethylsilyl isocyanate were used to prepare 670 mg (91% of theory, 92% purity) of the title compound. The reaction time here was about 16 h.

LC/MS (Method 1, ESIneg): Rₜ=1.69 min, m/z=512.18 [M−H]⁻.

### Example 262A

### tert-Butyl 2-carbamoyl-2-{[1-(cyanomethyl)-5-methyl-3-(1-methylcyclopropyl)-2,4-dioxo-1,2,3,4-tetrahydrothieno[2,3-d]pyrimidin-6-yl]methyl}hydrazinecarboxylate

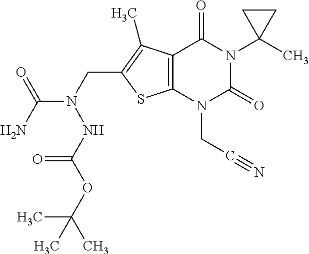 [SMILES: Cc1c(CN(NC(=O)OC(C)(C)C)C(N)=O)sc2c1c(=O)n(C1(C)CC1)c(=O)n2CC#N]

Analogously to the method described in Ex. 244A, 200 mg (0.439 mmol, 92% purity) of the compound from Ex. 218A and 118 μl (0.877 mmol) of trimethylsilyl isocyanate were used to prepare 125 mg (56% of theory, 92% purity) of the title compound. The reaction time here was about 16 h.

¹H-NMR (400 MHz, DMSO-d₆, δ/ppm): 8.92 (br. s, 1H), 6.20 (br. s, 2H), 5.08 (br. s, 2H), 4.59 (broad, 2H), 2.34 (br. s, 3H), 1.39 (br. s, 9H), 1.35 (s, 3H), 1.04-0.71 (m, 4H).

LC/MS (Method 1, ESIneg): Rₜ=1.46 min, m/z=461.16 [M−H]⁻.

### Example 263A

### tert-Butyl 2-carbamoyl-2-{[1-(2-methoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

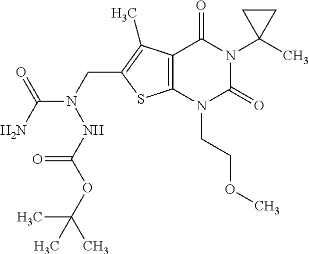 [SMILES: COCCn1c(=O)n(C2(C)CC2)c(=O)c2c(C)c(CN(NC(=O)OC(C)(C)C)C(N)=O)sc21]

To a solution of 660 mg (1.36 mmol, 91% purity) of the compound from Ex. 219A in 17 ml of isopropanol were added 314 mg (2.72 mmol) of trimethylsilyl isocyanate. After 24 h at RT, the volatile constituents of the reaction mixture were substantially removed on a rotary evaporator. 808 mg (100% of theory, 83% purity) of the title compound were obtained, which was used for subsequent reactions without further purification.

LC/MS (Method 2, ESIpos): Rₜ=0.77 min, m/z=482 [M+H]⁺.

### Example 264A

### tert-Butyl 2-carbamoyl-2-({5-methyl-3-(1-methylcyclopropyl)-2,4-dioxo-1-[2-(trifluoromethoxy)ethyl]-1,2,3,4-tetrahydrothieno[2,3-d]pyrimidin-6-yl}methyl)hydrazinecarboxylate

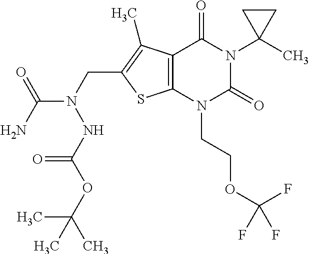 [SMILES: Cc1c(CN(NC(=O)OC(C)(C)C)C(N)=O)sc2c1c(=O)n(C1(C)CC1)c(=O)n2CCOC(F)(F)F]

To a solution of 290 mg (0.509 mmol, 86% purity) of the compound from Ex. 220A in 10 ml of isopropanol were added 137 μl (1.02 mmol) of trimethylsilyl isocyanate, and the mixture was stirred at RT for about 16 h. The reaction mixture was then concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and concentrated. The residue was purified by means of MPLC (Biotage Isolera One, SNAP KP-Sil cartridge, 50 g of silica gel, eluent: cyclohexane/ethyl acetate 1:1). After concentration of the product fractions and drying under high vacuum, 236 mg (81% of theory, 94% purity) of the title compound were obtained.

¹H-NMR (400 MHz, DMSO-d₆, δ/ppm): 8.88 (br. s, 1H), 6.17 (br. s, 2H), 4.57 (broad, 2H), 4.39 (t, 2H), 4.30-4.04 (m, 2H), 2.33 (s, 3H), 1.37 (br. s, 9H), 1.33 (s, 3H), 0.99-0.72 (m, 4H).

LC/MS (Method 1, ESIneg): Rₜ=1.74 min, m/z=534.16 [M−H]⁻.

### Example 265A

### tert-Butyl 2-carbamoyl-2-{[1-(2-ethoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

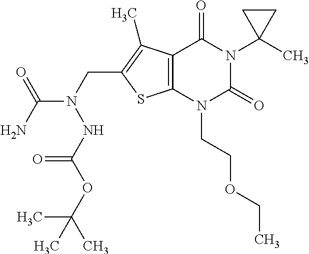 [SMILES: CCOCCn1c(=O)n(C2(C)CC2)c(=O)c2c(C)c(CN(NC(=O)OC(C)(C)C)C(N)=O)sc21]

Analogously to the method described in Ex. 264A, 200 mg (0.442 mmol) of the compound from Ex. 221A and 119 μl (0.884 mmol) of trimethylsilyl isocyanate were used to prepare 159 mg (72% of theory) of the title compound.

¹H-NMR (400 MHz, DMSO-d₆, δ/ppm): 8.88 (br. s, 1H), 6.16 (s, 2H), 5.00-4.18 (broad, 2H), 4.14-3.84 (m, 2H), 3.64 (t, 2H), 3.44 (q, 2H), 2.32 (s, 3H), 1.38 (br. s, 9H), 1.33 (s, 3H), 1.05 (t, 3H), 0.99-0.73 (m, 4H).

LC/MS (Method 2, ESIneg): Rₜ=0.85 min, m/z=494 [M−H]⁻.

### Example 266A

### tert-Butyl 2-carbamoyl-2-{[1-(2-isopropoxyethyl)-5-methyl-3-(1-methylcyclopropyl)-2,4-dioxo-1,2,3,4-tetrahydrothieno[2,3-d]pyrimidin-6-yl]methyl}hydrazinecarboxylate

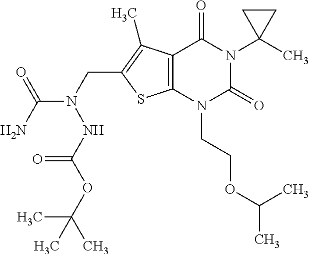 [SMILES: Cc1c(CN(NC(=O)OC(C)(C)C)C(N)=O)sc2c1c(=O)n(C1(C)CC1)c(=O)n2CCOC(C)C]

Analogously to the method described in Ex. 264A, 280 mg (0.600 mmol) of the compound from Ex. 222A and 161 μl (1.20 mmol) of trimethylsilyl isocyanate were used to prepare 258 mg (84% of theory) of the title compound.

¹H-NMR (400 MHz, DMSO-d₆, δ/ppm): 8.87 (br. s, 1H), 6.16 (br. s, 2H), 5.14-4.14 (broad, 2H), 4.10-3.81 (m, 2H), 3.64 (t, 2H), 3.60-3.50 (sept, 1H), 2.32 (s, 3H), 1.38 (br. s, 9H), 1.33 (s, 3H), 1.02 (d, 6H), 0.97-0.73 (m, 4H).

LC/MS (Method 1, ESIneg): Rₜ=1.69 min, m/z=508.22 [M−H]⁻.

### Example 267A

### tert-Butyl 2-carbamoyl-2-({5-methyl-3-(1-methylcyclopropyl)-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

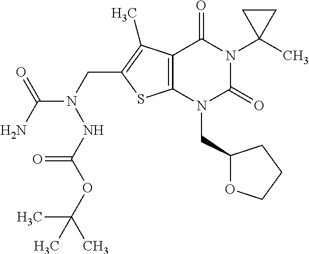 [SMILES: Cc1c(CN(NC(=O)OC(C)(C)C)C(N)=O)sc2c1c(=O)n(C1(C)CC1)c(=O)n2C[C@H]1CCCO1]

Analogously to the method described in Ex. 264A, 150 mg (0.323 mmol) of the compound from Ex. 223A and 87 μl (0.646 mmol) of trimethylsilyl isocyanate were used to prepare 157 mg (83% of theory, 87% purity) of the title compound.

LC/MS (Method 1, ESIneg): Rₜ=1.56 min, m/z=506.21 [M−H]⁻.

### Example 268A

### tert-Butyl 2-carbamoyl-2-({5-methyl-3-(1-methylcyclopropyl)-2,4-dioxo-1-[(2S)-tetrahydrofuran-2-ylmethyl]-1,2,3,4-tetrahydrothieno[2,3-d]pyrimidin-6-yl}methyl)hydrazinecarboxylate

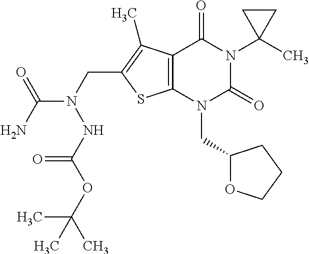 [SMILES: Cc1c(CN(NC(=O)OC(C)(C)C)C(N)=O)sc2c1c(=O)n(C1(C)CC1)c(=O)n2C[C@@H]1CCCO1]

Analogously to the method described in Ex. 264A, 265 mg (0.570 mmol) of the compound from Ex. 224A and 153 μl (1.14 mmol) of trimethylsilyl isocyanate were used to prepare 300 mg (98% of theory, 95% purity) of the title compound.

LC/MS (Method 1, ESIneg): Rt=1.55 min, m/z=506.21 [M−H]⁻.

### Example 269A

### tert-Butyl 2-carbamoyl-2-({1-(2-methoxyethyl)-5-methyl-2,4-dioxo-3-[1-(trifluoromethyl)cyclopropyl]-1,2,3,4-tetrahydrothieno[2,3-d]pyrimidin-6-yl}methyl)hydrazinecarboxylate

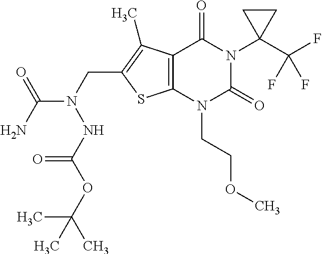 [SMILES: COCCn1c(=O)n(C2(C(F)(F)F)CC2)c(=O)c2c(C)c(CN(NC(=O)OC(C)(C)C)C(N)=O)sc21]

Analogously to the method described in Ex. 264A, 400 mg (0.812 mmol) of the compound from Ex. 269A and 218 μl (1.62 mmol) of trimethylsilyl isocyanate were used to prepare 492 mg (98% of theory, 87% purity) of the title compound. Purification of the product by means of MPLC was dispensed with here.

LC/MS (Method 2, ESIneg): Rₜ=0.87 min, m/z=534 [M−H]⁻.

### Example 270A

### tert-Butyl 2-carbamoyl-2-{[3-(1-ethylcyclopropyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

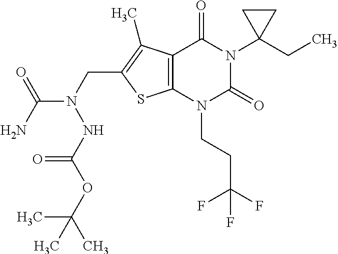 [SMILES: CCC1(n2c(=O)c3c(C)c(CN(NC(=O)OC(C)(C)C)C(N)=O)sc3n(CCC(F)(F)F)c2=O)CC1]

Analogously to the method described in Ex. 264A, 146 mg (0.238 mmol, 80% purity) of the compound from Ex. 226A and 64 μl (0.476 mmol) of trimethylsilyl isocyanate were used to prepare 119 mg (93% of theory) of the title compound.

¹H-NMR (400 MHz, DMSO-d₆, δ/ppm): 8.89 (br. s, 1H), 6.19 (br. s, 2H), 4.57 (broad, 2H), 4.09 (br. t, 2H), 2.82-2.63 (m, 2H), 2.33 (s, 3H), 1.69 (q, 2H), 1.38 (br. s, 9H), 1.03-0.87 (m, 2H), 0.84-0.75 (m, 2H), 0.81 (t, 3H).

### Example 271A

### tert-Butyl 2-carbamoyl-2-{[3-(1-ethylcyclopropyl)-1-(2-methoxyethyl)-5-methyl-2,4-dioxo-1,2,3,4-tetrahydrothieno[2,3-d]pyrimidin-6-yl]methyl}hydrazinecarboxylate

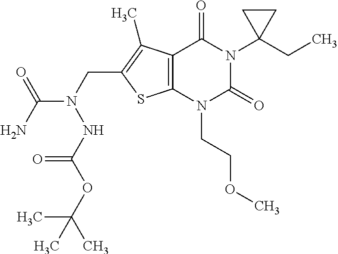 [SMILES: CCC1(n2c(=O)c3c(C)c(CN(NC(=O)OC(C)(C)C)C(N)=O)sc3n(CCOC)c2=O)CC1]

Analogously to the method described in Ex. 244A, 146 mg (0.323 mmol) of the compound from Ex. 227A and 87 μl (0.645 mmol) of trimethylsilyl isocyanate were used to prepare 168 mg (99% of theory, 95% purity) of the title compound. Purification of the product by means of MPLC was dispensed with here.

LC/MS (Method 1, ESIneg): Rₜ=1.62 min, m/z=494.21 [M−H]⁻.

### Example 272A

### tert-Butyl 2-carbamoyl-2-{[3-cyclobutyl-5-methyl-2,4-dioxo-1-(3,3,3-trifluoropropyl)-1,2,3,4-tetrahydrothieno[2,3-d]pyrimidin-6-yl]methyl}hydrazinecarboxylate

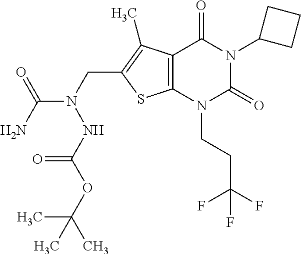 [SMILES: Cc1c(CN(NC(=O)OC(C)(C)C)C(N)=O)sc2c1c(=O)n(C1CCC1)c(=O)n2CCC(F)(F)F]

To a solution of 150 mg (0.302 mmol, 96% purity) of the compound from Ex. 228A in 9 ml of isopropanol were added 124 μl (0.907 mmol) of trimethylsilyl isocyanate, and the mixture was stirred at 50° C. for about 16 h. The reaction mixture was then concentrated and the residue was then separated into its components by means of preparative HPLC (Method 11). The combined product fractions were concentrated and the residue was dried under high vacuum. 121 mg (75% of theory) of the title compound were obtained.

¹H-NMR (400 MHz, DMSO-d₆, δ/ppm): 8.91 (br. s, 1H), 6.20 (br. s, 2H), 5.21 (quin, 1H), 5.07-4.20 (broad, 2H), 4.09 (t, 2H), 2.89-2.64 (m, 4H), 2.33 (s, 3H), 2.23-2.10 (m, 2H), 1.88-1.63 (m, 2H), 1.38 (br. s, 9H).

LC/MS (Method 1, ESIneg): Rₜ=1.90 min, m/z=518.17 [M−H]⁻.

### Example 273A

### tert-Butyl 2-carbamoyl-2-{[3-cyclobutyl-1-(2-methoxyethyl)-5-methyl-2,4-dioxo-1,2,3,4-tetrahydrothieno[2,3-d]pyrimidin-6-yl]methyl}hydrazinecarboxylate

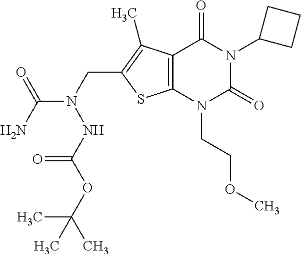 [SMILES: COCCn1c(=O)n(C2CCC2)c(=O)c2c(C)c(CN(NC(=O)OC(C)(C)C)C(N)=O)sc21]

To a solution of 150 mg (0.342 mmol) of the compound from Ex. 229A in 10 ml of isopropanol were added 141 μl (1.03 mmol) of trimethylsilyl isocyanate, and the mixture was stirred at 50° C. for about 16 h. The reaction mixture was then concentrated and the residue was then separated into its components by means of preparative HPLC (Method 11). The combined product fractions were concentrated and the residue was dried under high vacuum. 113 mg (68% of theory) of the title compound were obtained.

¹H-NMR (400 MHz, DMSO-d₆, δ/ppm): 8.91 (br. s, 1H), 6.18 (s, 2H), 5.22 (quin, 1H), 5.05-4.17 (broad, 2H), 4.00 (t, 2H), 3.63 (t, 2H), 3.25 (s, 3H), 2.90-2.76 (m, 2H), 2.32 (s, 3H), 2.16 (qt, 2H), 1.87-1.63 (m, 2H), 1.48-1.16 (br. s, 9H).

LC/MS (Method 1, ESIneg): Rₜ=1.70 min, m/z=480.19 [M−H]⁻.

### Example 274A

### tert-Butyl 2-carbamoyl-2-{[3-(3,3-difluorocyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

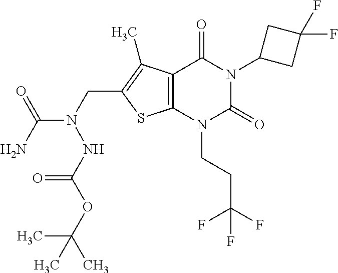 [SMILES: Cc1c(CN(NC(=O)OC(C)(C)C)C(N)=O)sc2c1c(=O)n(C1CC(F)(F)C1)c(=O)n2CCC(F)(F)F]

To a solution of 630 mg (1.23 mmol, 82% purity) of the compound from Ex. 230A in 16 ml of isopropanol were added 283 mg (2.46 mmol) of trimethylsilyl isocyanate. After a total of 30 h at RT, the volatile constituents of the reaction mixture were substantially removed on a rotary evaporator. 756 mg (87% of theory, 79% purity) of the title compound were obtained, which was used for subsequent reactions without further purification.

LC/MS (Method 2, ESIpos): Rₜ=1.00 min, m/z=556 [M+H]⁺.

### Example 275A

### tert-Butyl 2-carbamoyl-2-{[3-(3,3-difluorocyclobutyl)-1-(2-methoxyethyl)-5-methyl-2,4-dioxo-1,2,3,4-tetrahydrothieno[2,3-d]pyrimidin-6-yl]methyl}hydrazinecarboxylate

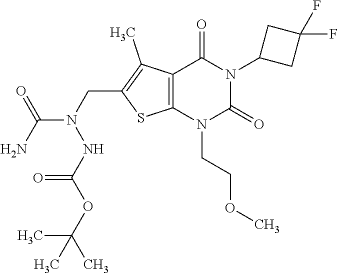 [SMILES: COCCn1c(=O)n(C2CC(F)(F)C2)c(=O)c2c(C)c(CN(NC(=O)OC(C)(C)C)C(N)=O)sc21]

To a solution of 195 mg (0.41 mmol) of the compound from Ex. 231A in 5 ml of isopropanol were added 94 mg (0.82 mmol) of trimethylsilyl isocyanate. After a total of 30 h at RT, the volatile constituents of the reaction mixture were substantially removed on a rotary evaporator. 232 mg (94% of theory, 86% purity) of the title compound were obtained, which was used for subsequent reactions without further purification.

LC/MS (Method 2, ESIpos): Rₜ=0.90 min, m/z=518 [M+H]⁺.

### Example 276A

### tert-Butyl 2-carbamoyl-2-{[5-methyl-3-(1-methylcyclobutyl)-2,4-dioxo-1-(3,3,3-trifluoropropyl)-1,2,3,4-tetrahydrothieno[2,3-d]pyrimidin-6-yl]methyl}hydrazinecarboxylate

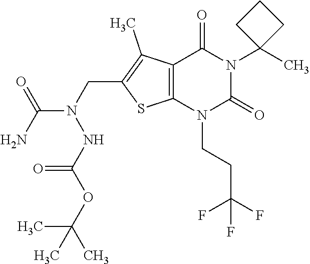 [SMILES: Cc1c(CN(NC(=O)OC(C)(C)C)C(N)=O)sc2c1c(=O)n(C1(C)CCC1)c(=O)n2CCC(F)(F)F]

Analogously to the method described in Ex. 264A, 325 mg (0.663 mmol) of the compound from Ex. 232A and 178 μl (1.33 mmol) of trimethylsilyl isocyanate were used to prepare 415 mg (99% of theory, 85% purity) of the title compound. Purification of the product by means of MPLC was dispensed with here.

LC/MS (Method 1, ESIneg): Rₜ=1.95 min, m/z=532.18 [M−H]⁻.

### Example 277A

### tert-Butyl 2-carbamoyl-2-{[1-(2-methoxyethyl)-5-methyl-3-(1-methylcyclobutyl)-2,4-dioxo-1,2,3,4-tetrahydrothieno[2,3-d]pyrimidin-6-yl]methyl}hydrazinecarboxylate

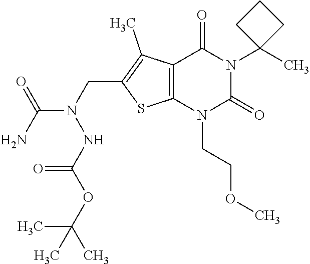 [SMILES: COCCn1c(=O)n(C2(C)CCC2)c(=O)c2c(C)c(CN(NC(=O)OC(C)(C)C)C(N)=O)sc21]

Analogously to the method described in Ex. 264A, 300 mg (0.663 mmol) of the compound from Ex. 233A and 178 μl (1.33 mmol) of trimethylsilyl isocyanate were used to prepare 388 mg (96% of theory, 82% purity) of the title compound. Purification of the product by means of MPLC was dispensed with here.

LC/MS (Method 1, ESIneg): Rₜ=1.72 min, m/z=494.21 [M−H]⁻.

### Example 278A

### tert-Butyl 2-carbamoyl-2-{[3-(trans-3-methoxycyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

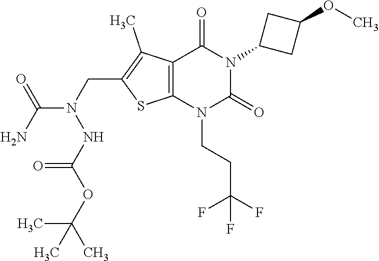 [SMILES: CO[C@H]1C[C@H](n2c(=O)c3c(C)c(CN(NC(=O)OC(C)(C)C)C(N)=O)sc3n(CCC(F)(F)F)c2=O)C1]

To a solution of 1.11 g (1.38 mmol, 63% purity) of the compound from Ex. 234A in 17 ml of isopropanol were added 317 mg (2.80 mmol) of trimethylsilyl isocyanate. After a total of 72 h at RT, the volatile constituents of the reaction mixture were substantially removed on a rotary evaporator. 1.09 g (89% of theory, 62% purity) of the title compound were obtained, which was used for subsequent reactions without further purification.

LC/MS (Method 2, ESIpos): Rₜ=0.94 min, m/z=550 [M+H]⁺.

### Example 279A

### tert-Butyl 2-carbamoyl-2-{[3-(trans-3-methoxycyclobutyl)-1-(2-methoxyethyl)-5-methyl-2,4-dioxo-1,2,3,4-tetrahydrothieno[2,3-d]pyrimidin-6-yl]methyl}hydrazinecarboxylate

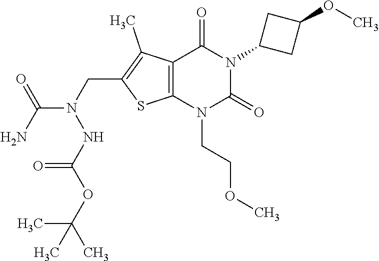 [SMILES: COCCn1c(=O)n([C@H]2C[C@H](OC)C2)c(=O)c2c(C)c(CN(NC(=O)OC(C)(C)C)C(N)=O)sc21]

To a solution of 1.02 g (1.60 mmol, 74% purity) of the compound from Ex. 235A in 20 ml of isopropanol were added 369 mg (3.20 mmol) of trimethylsilyl isocyanate. After a total of 72 h at RT, the volatile constituents of the reaction mixture were substantially removed on a rotary evaporator. 1.21 g (79% of theory, 54% purity) of the title compound were obtained, which was used for subsequent reactions without further purification.

LC/MS (Method 2, ESIpos): Rₜ=0.82 min, m/z=512 [M+H]⁺.

### Example 280A

### tert-Butyl 2-carbamoyl-2-{[3-(cis-3-methoxycyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

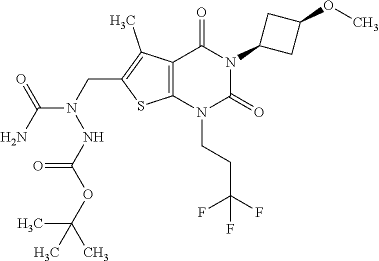 [SMILES: CO[C@H]1C[C@@H](n2c(=O)c3c(C)c(CN(NC(=O)OC(C)(C)C)C(N)=O)sc3n(CCC(F)(F)F)c2=O)C1]

To a solution of 0.51 g (0.77 mmol, 76% purity) of the compound from Ex. 236A in 10 ml of isopropanol were added 177 mg (1.54 mmol) of trimethylsilyl isocyanate. After a total of 32 h at RT, the volatile constituents of the reaction mixture were substantially removed on a rotary evaporator. 0.62 g (78% of theory, 53% purity) of the title compound was obtained, which was used for subsequent reactions without further purification.

### Example 281A

### tert-Butyl 2-carbamoyl-2-{[3-(cis-3-methoxycyclobutyl)-1-(2-methoxyethyl)-5-methyl-2,4-dioxo-1,2,3,4-tetrahydrothieno[2,3-d]pyrimidin-6-yl]methyl}hydrazinecarboxylate

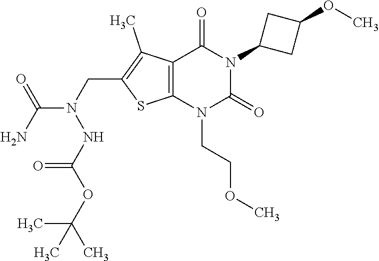 [SMILES: COCCn1c(=O)n([C@H]2C[C@@H](OC)C2)c(=O)c2c(C)c(CN(NC(=O)OC(C)(C)C)C(N)=O)sc21]

To a solution of 0.48 g (0.83 mmol, 60% purity) of the compound from Ex. 237A in 10 ml of isopropanol were added 190 mg (1.65 mmol) of trimethylsilyl isocyanate. After a total of 72 h at RT, a further 50 μl (0.37 mmol) of trimethylsilyl isocyanate were added, and the reaction mixture were stirred at RT for another 16 h. The volatile constituents of the reaction mixture were then substantially removed on a rotary evaporator. 0.48 g (60% of theory, 52% purity) of the title compound was obtained, which was used for subsequent reactions without further purification.

### Example 282A

### tert-Butyl 2-carbamoyl-2-{[3-(3,3-dimethylcyclobutyl)-5-methyl-2,4-dioxo-1-(3,3,3-trifluoropropyl)-1,2,3,4-tetrahydrothieno[2,3-d]pyrimidin-6-yl]methyl}hydrazinecarboxylate

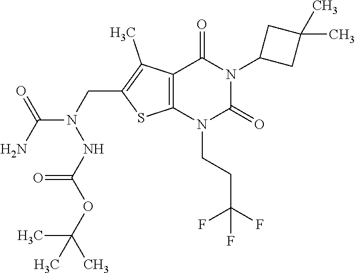 [SMILES: Cc1c(CN(NC(=O)OC(C)(C)C)C(N)=O)sc2c1c(=O)n(C1CC(C)(C)C1)c(=O)n2CCC(F)(F)F]

To a solution of 395 mg (0.64 mmol, 83% purity) of the compound from Ex. 238A in 8 ml of isopropanol were added 148 mg (1.28 mmol) of trimethylsilyl isocyanate. After a total of 72 h at RT, the volatile constituents of the reaction mixture were substantially removed on a rotary evaporator. 0.39 g (95% of theory, 85% purity) of the title compound was obtained, which was used for subsequent reactions without further purification.

### Example 283A

### tert-Butyl 2-carbamoyl-2-{[3-(3,3-dimethylcyclobutyl)-1-(2-methoxyethyl)-5-methyl-2,4-dioxo-1,2,3,4-tetrahydrothieno[2,3-d]pyrimidin-6-yl]methyl}hydrazinecarboxylate

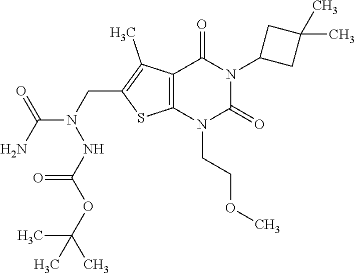 [SMILES: COCCn1c(=O)n(C2CC(C)(C)C2)c(=O)c2c(C)c(CN(NC(=O)OC(C)(C)C)C(N)=O)sc21]

To a solution of 595 mg (1.0 mmol, 56% purity) of the compound from Ex. 239A in 13 ml of isopropanol were added 229 mg (2.0 mmol) of trimethylsilyl isocyanate. After a total of 72 h at RT, the volatile constituents of the reaction mixture were substantially removed on a rotary evaporator. 0.59 g (70% of theory, 59% purity) of the title compound was obtained, which was used for subsequent reactions without further purification.

### Example 284A

### tert-Butyl 2-carbamoyl-2-{[5-methyl-2,4-dioxo-3-(spiro[3.3]hept-2-yl)-1-(3,3,3-trifluoropropyl)-1,2,3,4-tetrahydrothieno[2,3-d]pyrimidin-6-yl]methyl}hydrazinecarboxylate

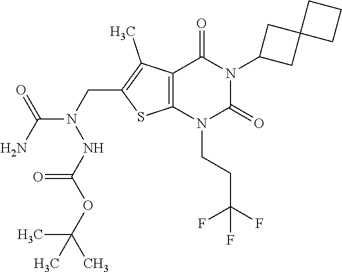 [SMILES: Cc1c(CN(NC(=O)OC(C)(C)C)C(N)=O)sc2c1c(=O)n(C1CC3(CCC3)C1)c(=O)n2CCC(F)(F)F]

Analogously to the method described in Ex. 264A, 425 mg (0.823 mmol) of the compound from Ex. 240A and 221 μl (1.65 mmol) of trimethylsilyl isocyanate were used to prepare 460 mg (99% of theory) of the title compound.

¹H-NMR (400 MHz, DMSO-d₆, δ/ppm): 8.90 (br. s, 1H), 6.20 (br. s, 2H), 5.05 (quin, 1H), 4.93-4.17 (broad, 2H), 4.08 (t, 2H), 2.87-2.63 (m, 4H), 2.32 (s, 3H), 2.29-2.20 (m, 2H), 2.07 (t, 2H), 2.01-1.93 (m, 2H), 1.87-1.74 (m, 2H), 1.38 (br. s, 9H).

LC/MS (Method 2, ESIneg): Rₜ=1.17 min, m/z=558 [M−H]⁻.

### Example 285A

### tert-Butyl 2-carbamoyl-2-{[1-(2-methoxyethyl)-5-methyl-2,4-dioxo-3-(spiro[3.3]hept-2-yl)-1,2,3,4-tetrahydrothieno[2,3-d]pyrimidin-6-yl]methyl}hydrazinecarboxylate

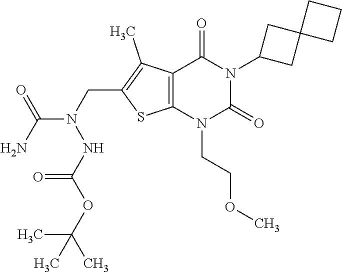 [SMILES: COCCn1c(=O)n(C2CC3(CCC3)C2)c(=O)c2c(C)c(CN(NC(=O)OC(C)(C)C)C(N)=O)sc21]

Analogously to the method described in Ex. 264A, 380 mg (0.794 mmol) of the compound from Ex. 241A and 213 μl (1.59 mmol) of trimethylsilyl isocyanate were used to prepare 380 mg (91% of theory) of the title compound.

¹H-NMR (400 MHz, DMSO-d₆, δ/ppm): 8.90 (br. s, 1H), 6.17 (br. s, 2H), 5.06 (quin, 1H), 4.55 (broad, 2H), 3.99 (t, 2H), 3.62 (t, 2H), 3.24 (s, 3H), 2.83 (td, 2H), 2.30 (s, 3H), 2.24 (td, 2H), 2.10-2.03 (m, 2H), 2.02-1.93 (m, 2H), 1.88-1.75 (m, 2H), 1.38 (br. s, 9H).

LC/MS (Method 2, ESIneg): Rₜ=1.07 min, m/z=520 [M−H]⁻.

### Example 286A

### tert-Butyl 2-carbamoyl-2-{[3-cyclopentyl-5-methyl-2,4-dioxo-1-(3,3,3-trifluoropropyl)-1,2,3,4-tetrahydrothieno[2,3-d]pyrimidin-6-yl]methyl}hydrazinecarboxylate

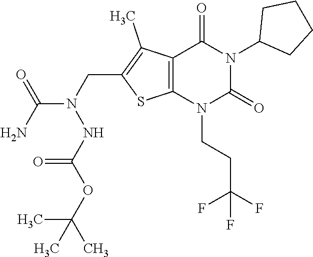 [SMILES: Cc1c(CN(NC(=O)OC(C)(C)C)C(N)=O)sc2c1c(=O)n(C1CCCC1)c(=O)n2CCC(F)(F)F]

To a solution of 435 mg (0.782 mmol, 88% purity) of the compound from Ex. 242A in 20 ml of isopropanol were added 212 μl (1.56 mmol) of trimethylsilyl isocyanate, and the mixture was stirred at RT for 2.5 days. The reaction mixture was then concentrated to dryness. The remaining residue was stirred with a little methanol at RT. The solids were filtered off with suction and, after drying under high vacuum, gave 290 mg (69% of theory) of the title compound.

¹H-NMR (400 MHz, DMSO-d₆, δ/ppm): 8.90 (br. s, 1H), 6.20 (s, 2H), 5.30 (t, 1H), 5.11-4.17 (broad, 1H), 4.10 (t, 2H), 2.81-2.69 (m, 2H), 2.33 (s, 3H), 2.06-1.96 (m, 2H), 1.96-1.84 (m, 2H), 1.79-1.69 (m, 2H), 1.59-1.51 (m, 2H), 1.38 (br. s, 9H).

LC/MS (Method 1, ESIneg): Rₜ=2.02 min, m/z=532.18 [M−H]⁻.

### Example 287A

### tert-Butyl 2-carbamoyl-2-{[3-cyclopentyl-1-(2-methoxyethyl)-5-methyl-2,4-dioxo-1,2,3,4-tetrahydrothieno[2,3-d]pyrimidin-6-yl]methyl}hydrazinecarboxylate

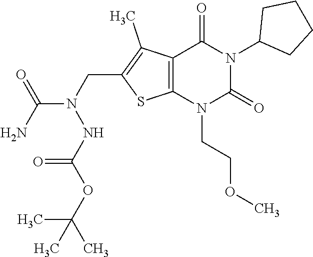 [SMILES: COCCn1c(=O)n(C2CCCC2)c(=O)c2c(C)c(CN(NC(=O)OC(C)(C)C)C(N)=O)sc21]

Analogously to the method described in Ex. 244A, 494 mg (0.967 mmol, 88% purity) of the compound from Ex. 243A and 259 μl (1.93 mmol) of trimethylsilyl isocyanate were used to prepare 460 mg (87% of theory, 91% purity) of the title compound.

¹H-NMR (400 MHz, DMSO-d₆, δ/ppm): 8.90 (br. s, 1H), 6.18 (s, 2H), 5.30 (quin, 1H), 4.98-4.17 (broad, 2H), 4.01 (t, 2H), 3.63 (t, 2H), 3.24 (s, 3H), 2.32 (s, 3H), 2.07-1.96 (m, 2H), 1.95-1.83 (m, 2H), 1.81-1.67 (m, 2H), 1.62-1.49 (m, 2H), 1.39 (br. s, 9H).

LC/MS (Method 1, ESIneg): Rₜ=1.78 min, m/z=494.21 [M−H]⁻.

### Example 288A

### Diethyl 5-[(cyclopropylcarbamoyl)amino]-3-methylthiophene-2,4-dicarboxylate

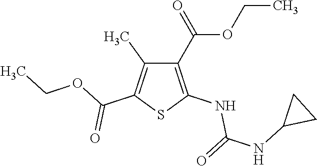 [SMILES: CCOC(=O)c1sc(NC(=O)NC2CC2)c(C(=O)OCC)c1C]

To a solution of 3.0 g (11.3 mmol, 97% purity) of diethyl 5-amino-3-methylthiophene-2,4-dicarboxylate in 12 ml of pyridine were added 3.2 ml (45.2 mmol, 98% purity) of cyclopropyl isocyanate, and the mixture was heated to 80° C. for about 16 h. Subsequently, the reaction mixture was concentrated to dryness. The residue that remained was taken up in dichloromethane three times and concentrated again each time. Then the residue was taken up in ethyl acetate and washed with water. After reconcentration, the solids obtained were stirred in a mixture of 200 ml of diisopropyl ether, 20 ml of ethyl acetate and 20 ml of dichloromethane at RT for 2 h. Then the solids were filtered off with suction and dried under high vacuum. This gave a first fraction of 3.05 g of the title compound. The mother liquor from the stirring was concentrated, and a further 0.8 g of the title compound was isolated from this residue by means of MPLC (Biotage Isolera One, SNAP KP-Sil cartridge, 10 g of silica gel, eluent: cyclohexane/ethyl acetate 2:1). A total of 3.85 g (99% of theory) of the title compound was thus obtained.

¹H-NMR (400 MHz, DMSO-d₆, δ/ppm): 10.52 (br. s, 1H), 8.21 (br. s, 1H), 4.32 (q, 2H), 4.23 (q, 2H), 2.66 (s, 3H), 2.62-2.55 (m, 1H, partially concealed by DMSO signal), 1.32 (t, 3H), 1.28 (t, 3H), 0.76-0.62 (m, 2H), 0.52-0.41 (m, 2H).

LC/MS (Method 1, ESIpos): Rₜ=2.05 min, m/z=341.12 [M+H]⁺.

### Example 289A

### Ethyl 3-cyclopropyl-5-methyl-2,4-dioxo-1,2,3,4-tetrahydrothieno[2,3-d]pyrimidine-6-carboxylate

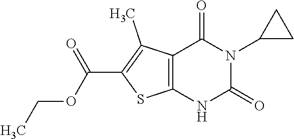 [SMILES: CCOC(=O)c1sc2[nH]c(=O)n(C3CC3)c(=O)c2c1C]

3.85 g (11.3 mmol) of the compound from Ex. 288A were dissolved in 96 ml of ethanol, and 7.5 ml (20.1 mmol) of a 21% solution of sodium ethoxide in ethanol were added. The reaction mixture was stirred at RT for about 15 h. Thereafter, the mixture was concentrated to about half its original volume and then acidified by adding 1 M hydrochloric acid. In the course of this, the product precipitated out. The product was filtered off with suction, washed to neutrality with water and dried under high vacuum. 3.06 g (91% of theory) of the title compound were obtained.

¹H-NMR (400 MHz, DMSO-d₆, δ/ppm): 12.25 (s, 1H), 4.25 (q, 2H), 2.72 (s, 3H), 2.58-2.51 (m, 1H, partially concealed by DMSO signal), 1.28 (t, 3H), 1.03-0.95 (m, 2H), 0.73-0.65 (m, 2H).

LC/MS (Method 1, ESIpos): Rₜ=1.48 min, m/z=295.07 [M+H]⁺.

### Example 290A

### Ethyl 3-cyclopropyl-5-methyl-2,4-dioxo-1-(3,3,3-trifluoropropyl)-1,2,3,4-tetrahydrothieno[2,3-d]pyrimidine-6-carboxylate

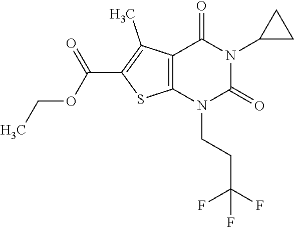 [SMILES: CCOC(=O)c1sc2c(c1C)c(=O)n(C1CC1)c(=O)n2CCC(F)(F)F]

1.0 g (3.40 mmol) of the compound from Ex. 289A and 1.17 g (8.49 mmol) of potassium carbonate were stirred in 25 ml of anhydrous DMF at RT for 15 min, before 2.28 g (10.2 mmol) of 1,1,1-trifluoro-3-iodopropane were added. Then the reaction mixture was stirred at 50° C. for about 16 h. After cooling to RT, water was added thereto and it was stirred at RT for 30 min. The product which precipitated out was filtered off with suction, washed with a little water and dried under high vacuum. 1.23 g (91% of theory) of the title compound were obtained.

¹H-NMR (400 MHz, DMSO-d₆, δ/ppm): 4.29 (q, 2H), 4.12 (t, 2H), 2.87-2.68 (m, 2H), 2.76 (s, 3H), 2.65-2.58 (m, 1H), 1.30 (t, 3H), 1.08-0.98 (m, 2H), 0.73-0.64 (m, 2H).

LC/MS (Method 1, ESIpos): Rₜ=2.06 min, m/z=391.09 [M+H]⁺.

### Example 291A

### Ethyl 3-cyclopropyl-1-(2-methoxyethyl)-5-methyl-2,4-dioxo-1,2,3,4-tetrahydrothieno[2,3-d]pyrimidine-6-carboxylate

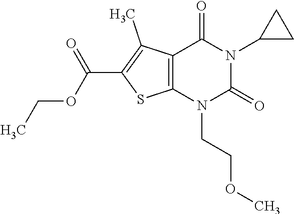 [SMILES: CCOC(=O)c1sc2c(c1C)c(=O)n(C1CC1)c(=O)n2CCOC]

Analogously to the method described in Ex. 290A, 1.0 g (3.40 mmol) of the compound from Ex. 289A and 1.89 g (13.6 mmol) of 2-bromoethyl methyl ether were used to prepare 1.13 g (92% of theory) of the title compound.

¹H-NMR (400 MHz, DMSO-d₆, δ/ppm): 4.27 (q, 2H), 4.04 (t, 2H), 3.63 (t, 2H), 3.24 (s, 3H), 2.75 (s, 3H), 2.65-2.58 (m, 1H), 1.29 (t, 3H), 1.06-0.97 (m, 2H), 0.74-0.65 (m, 2H).

LC/MS (Method 1, ESIpos): Rₜ=1.83 min, m/z=353.12 [M+H]⁺.

### Example 292A

### 3-Cyclopropyl-6-[dideutero(hydroxy)methyl]-5-methyl-1-(3,3,3-trifluoropropyl)thieno[2,3-d]pyrimidine-2,4(1H,3H)-dione

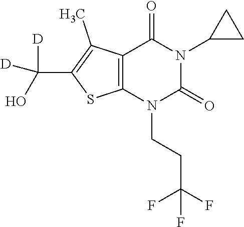 [SMILES: [2H]C([2H])(O)c1sc2c(c1C)c(=O)n(C1CC1)c(=O)n2CCC(F)(F)F]

To a solution of 1.20 g (3.01 mmol) of the compound from Ex. 290A in 27 ml of THF were added dropwise, at −78° C., 2.71 ml (2.71 mmol) of a 1 M solution of lithium aluminium deuteride in THF. The reaction mixture was then stirred at 0° C. for 1 h. Then 1.2 ml of water, 9 ml of 1 M sodium hydroxide solution and a little kieselguhr were added, and the cooling bath was removed. The precipitate formed was filtered off with suction and washed thoroughly with THF. The filtrate combined with the wash liquid was concentrated to dryness. The residue that remained here was taken up in ethyl acetate and washed successively with water and saturated aqueous sodium chloride solution. After drying over anhydrous magnesium sulfate, the mixture was filtered and concentrated. The crude product was stirred with a little dichloromethane at RT. The solids were removed and dried under reduced pressure. The mother liquor was concentrated and the residue was purified by means of MPLC (Biotage Isolera One, SNAP KP-Sil cartridge, 50 g of silica gel, eluent: cyclohexane/ethyl acetate 9:1-0:1). The product fraction was concentrated and combined with the solids isolated beforehand. This gave a total of 723 mg (65% of theory, 95% purity) of the title compound.

¹H-NMR (400 MHz, DMSO-d₆, δ/ppm): 5.54 (s, 1H), 4.08 (t, 2H), 2.74 (qt, 2H), 2.60 (tt, 1H), 2.32 (s, 3H), 1.06-0.96 (m, 2H), 0.73-0.63 (m, 2H).

LC/MS (Method 1, ESIpos): Rₜ=1.39 min, m/z=351.09 [M+H]⁺.

### Example 293A

### 3-Cyclopropyl-6-[dideutero(hydroxy)methyl]-1-(2-methoxyethyl)-5-methylthieno[2,3-d]pyrimidine-2,4(1H,3H)-dione

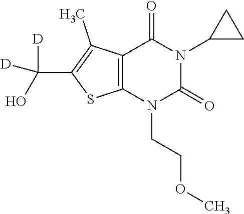 [SMILES: [2H]C([2H])(O)c1sc2c(c1C)c(=O)n(C1CC1)c(=O)n2CCOC]

Analogously to the process described in Ex. 292A, 1.12 g (3.12 mmol) of the compound from Ex. 291A and 3.8 ml (3.80 mmol) of a 1 M lithium aluminium deuteride solution were used to prepare 570 mg (58% of theory) of the title compound.

¹H-NMR (400 MHz, DMSO-d₆, δ/ppm): 5.48 (s, 1H), 4.00 (t, 2H), 3.62 (t, 2H), 3.24 (s, 3H), 2.64-2.56 (m, 1H), 2.30 (s, 3H), 1.05-0.96 (m, 2H), 0.71-0.63 (m, 2H).

LC/MS (Method 2, ESIpos): Rₜ=0.66 min, m/z=313 [M+H]⁺.

### Example 294A

### Ethyl 4-methyl-2-{[(2-methylcyclopropyl)carbamoyl]amino}thiophene-3-carboxylate (mixture of trans and cis racemate)

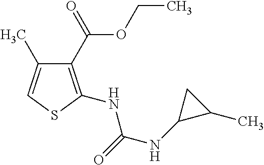

1-Isocyanato-2-methylcyclopropane: To a solution of 16.05 g (157 mmol, 98% purity) of commercially available 2-methylcyclopropanecarboxylic acid, typically consisting to an extent of about 85% of trans racemate and to an extent of about 15% of cis racemate, and 22 ml (157 mmol) of triethylamine in 300 ml of toluene were added dropwise 34 ml (157 mmol) of diphenyl phosphorazidate (DPPA). After the dropwise addition had ended, the mixture was heated to reflux for 2 h and then cooled down to RT.

Title compound: In a second reaction vessel, 10.0 g (52.4 mmol, 97% purity) of ethyl 2-amino-4-methylthiophene-3-carboxylate were dissolved in 67 ml of pyridine, and the isocyanate solution in toluene prepared above was added thereto. Subsequently, the reaction mixture was stirred at 80° C. for about 18 h. After cooling to RT, the mixture was diluted with about 1.2 litres of ethyl acetate and washed successively with water, 0.5 M hydrochloric acid, 5% aqueous ammonia and saturated aqueous sodium chloride solution. After drying over anhydrous magnesium sulfate, the mixture was filtered and the concentrated. The remaining residue was purified by means of suction filtration through 350 g of silica gel (eluent: cyclohexane/ethyl acetate 9:1→2:1). After combining the product fractions, concentration and drying of the residue under high vacuum, 14.26 g (94% of theory, 98% purity) of the title compound were obtained as a mixture of about 85% trans racemate and about 15% cis racemate.

LC/MS (Method 2, ESIpos): Rₜ=1.01 min, m/z=283 [M+H]⁺, 15.2% area; Rₜ=1.04 min, m/z=283 [M+H]⁺, 82.8% area.

### Example 295A

### 5-Methyl-3-(2-methylcyclopropyl)thieno[2,3-d]pyrimidine-2,4(1H,3H)-dione (trans racemate)

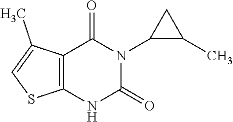 [SMILES: Cc1csc2[nH]c(=O)n(C3CC3C)c(=O)c12]

4.80 g (17.0 mmol) of the compound from Ex. 294A were dissolved in 54 ml of ethanol, and 12.7 ml (34.0 mmol) of a solution of sodium ethoxide (21%) in ethanol were added. After the mixture had been stirred at RT for about 16 h, it was diluted with 65 ml of water and brought to pH 3 with 1 M hydrochloric acid while stirring vigorously. The solid that precipitated out was filtered off, washed to neutrality with 50 ml of water and dried. Subsequently, the solids were stirred with 125 ml of acetonitrile at RT for about 18 h. Filtration with suction and drying under high vacuum gave 3.08 g (74% of theory, 97% purity, remainder: cis racemate) of the title compound, which is identical to the compound from Ex. 19A.

¹H-NMR (400 MHz, DMSO-d₆, δ/ppm): 11.91 (s, 1H), 6.63 (d, 1H), 2.33 (d, 3H), 2.17 (dt, 1H), 1.14 (d, 3H), 1.04-0.92 (m, 1H), 0.88-0.75 (m, 2H).

LC/MS (Method 1, ESIpos): Rₜ=1.32 min, m/z=237.07 [M+H]⁺.

### Example 296A

### 5-Methyl-3-(2-methylcyclopropyl)thieno[2,3-d]pyrimidine-2,4(1H,3H)-dione (cis racemate)

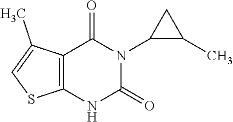 [SMILES: Cc1csc2[nH]c(=O)n(C3CC3C)c(=O)c12]

The aqueous filtrate from the preparation of the compound from Ex. 295A was extracted with ethyl acetate. The organic extract was dried over anhydrous magnesium sulfate, filtered and concentrated. Drying of the residue under high vacuum gave 0.90 g (21% of theory, 95% purity; remainder: trans racemate) of the title compound.

¹H-NMR (400 MHz, DMSO-d₆, δ/ppm): 11.95 (br. s, 1H), 6.64 (d, 1H), 2.60-2.53 (m, 1H, partially concealed by DMSO signal), 2.33 (d, 3H), 1.26-1.16 (m, 2H), 0.85 (d, 3H), 0.63-0.48 (m, 1H).

LC/MS (Method 1, ESIpos): Rₜ=1.27 min, m/z=237.07 [M+H]⁺.

### Example 297A

### 5-Methyl-3-(2-methylcyclopropyl)-2,4-dioxo-1,2,3,4-tetrahydrothieno[2,3-d]pyrimidine-6-carbaldehyde (cis racemate)

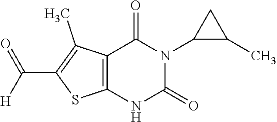 [SMILES: Cc1c(C=O)sc2[nH]c(=O)n(C3CC3C)c(=O)c12]

Analogously to the process described in Ex. 37A, 780 mg (3.30 mmol) of the compound from Ex. 296A, 3.7 ml (39.6 mmol) of phosphorus oxychloride and 3.6 ml (46.2 mmol) of DMF were used to prepare 688 mg (74% of theory, 95% purity) of the title compound. A difference was that the reaction time after the addition of phosphorus oxychloride here was 60 min.

¹H-NMR (400 MHz, DMSO-d₆, δ/ppm): 12.44 (s, 1H), 10.05 (s, 1H), 2.74 (s, 3H), 2.58 (td, 1H), 1.28-1.16 (m, 2H), 0.87 (d, 3H), 0.60-0.49 (m, 1H).

LC/MS (Method 1, ESIpos): Rₜ=1.20 min, m/z=265.06 [M+H]⁺.

### Example 298A

### (2R)-1-Hydroxy-3-phenylpropan-2-aminium (1S,2S)-2-methylcyclopropanecarboxylate

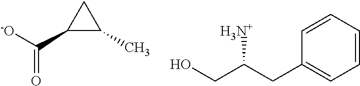

Analogously to a known process [see WO 2008/103185-A2], 660 g (6.59 mol) of commercially available 2-methylcyclopropanecarboxylic acid, which typically consists to an extent of 85% of trans racemate and to an extent of about 15% of cis racemate, in 6.6 were initially charged, and 508 g (3.36 mol) of (2R)-2-amino-3-phenylpropan-1-ol (R-phenylalaninol) were added. A thick white precipitate flocculated out here. The heterogeneous mixture was heated to 70° C. and stirred at this temperature for 2 h. Over this time, the precipitate went almost completely into solution. The heating was switched off and the mixture was gradually cooled down to RT while stirring. After about 16 h, the solids that had precipitated out again were filtered off with suction and washed twice with 500 ml each time of ethyl acetate. The precipitate was dried under reduced pressure and then divided into two equal portions, with which the procedure as described below was run in exactly the same way: Each of the two portions of solids was taken up in 5.1 litres of ethyl acetate and heated to reflux for 2 h. Over this time, the solids almost completely went back into solution again. Then the heating was switched off again and the mixture was gradually cooled down to RT while stirring. After about 16 h, the reprecipitated solids were filtered off with suction and washed twice with 250 ml each time of ethyl acetate. The precipitate was dried again under reduced pressure. This recrystallizating operation was repeated once more: The solids were suspended in 2.6 litres of ethyl acetate and heated again to reflux for 2 h. In this case, a significant amount of the solids remained undissolved. The mixture was gradually cooled down again to RT while stirring. After 2 days, the solids were filtered off with suction, washed twice with 210 ml each time of ethyl acetate and then dried under reduced pressure. The two solids fractions thus treated were combined and together gave 309 g (18% of theory) of the title compound.

¹H-NMR (400 MHz, DMSO-d₆, δ/ppm): 7.33-7.25 (m, 2H), 7.24-7.14 (m, 3H), 5.50 (broad, ca. 4H), 3.39-3.29 (m, 1H), 3.27-3.17 (m, 1H), 2.97 (br. s, 1H), 2.77-2.65 (m, 1H), 2.61-2.52 (m, 1H), 1.21-1.06 (m, 2H), 1.02 (d, 3H), 0.87 (dt, 1H), 0.53-0.47 (m, 1H).

LC/MS (Method 8, ESIpos): Rₜ=0.98 min, m/z=152 [M+H]⁺ (phenylalaninol).

### Example 299A

### (1S,2S)-2-Methylcyclopropanecarboxylic acid

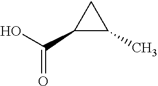

To 2.46 litres of 1 M hydrochloric acid were added 309 g (1.23 mol) of the compound from Ex. 298A. The mixture was stirred at RT for about 10 min. Then the mixture was extracted twice with 1.22 litres each time of ethyl acetate. The combined organic extracts were washed once with water and dried over anhydrous magnesium sulfate. After filtration, the mixture was concentrated and the residue was dried briefly under high vacuum. 115 g (93% of theory) of the title compound were obtained.

¹H-NMR (400 MHz, DMSO-d₆, δ/ppm): 11.94 (s, 1H), 1.27-1.15 (m, 2H), 1.05 (d, 3H), 0.99-0.92 (m, 1H), 0.65 (ddd, 1H).

LC/MS (Method 8, ESIneg): Rₜ=0.26 min, m/z=99 [M−H].

Specific optical rotation: [c]D₂₀=+95.9°·ml·dm⁻¹·g⁻¹ (ethanol).

### Example 300A

### (1S,2S)-1-Isocyanato-2-methylcyclopropane

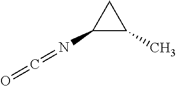

To an initial charge of 156 g (1.56 mol) of the compound from Ex. 299A in 2.1 litres of toluene were added 206 ml (1.48 mol) of triethylamine. Then the reaction vessel was immersed into an oil bath preheated to 85° C. Once the internal temperature had reached 60° C., the dropwise addition of 407 g (1.48 mol) of diphenyl phosphorazidate (DPPA) was commenced. From about 75° C., controlled evolution of nitrogen set in, and the reaction mixture heated up to 95° C. as a result of the exothermic reaction. The oil bath that was on a labjack was lowered from the flask, and the rate of dropwise addition of the DPPA was adjusted such that the internal temperature settled at about 95-100° C. After the dropwise addition had ended, the oil bath was raised back to the flask and the reaction mixture was stirred at 85° C. for a further 15 min. After cooling down to about 40° C., the solution of the title compound thus obtained was used for further reactions (see Ex. 301A).

### Example 301A

### Ethyl 4-methyl-2-({[(1S,2S)-2-methylcyclopropyl]carbamoyl}amino)thiophene-3-carboxylate

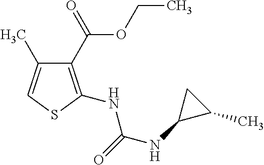

A solution of 211 g (1.14 mol) of ethyl 2-amino-4-methylthiophene-3-carboxylate in 1.37 litres of pyridine was heated to 50° C. and, over a period of 15 min, the isocyanate solution from Ex. 300A (1.48 mol, assuming conversion of 100%) was added. Subsequently, the reaction mixture was stirred at 80° C. for about 18 h. After cooling to RT, the mixture was diluted with 1.2 litres of ethyl acetate and successively extracted by shaking with the following aqueous phases: four times 4 litres of 2 M hydrochloric acid, 4 litres of 5% aqueous ammonia, 4 litres of 10% sodium chloride solution. After drying over anhydrous magnesium sulfate, the mixture was filtered and concentrated. The residue that remained was purified by means of chromatography on silica gel (0.063-0.2 mm, 12 kg) with dichloromethane/acetone (98:2, 120 litres) as eluent. After concentration of the product fractions and drying under high vacuum, 293 g (91% of theory) of the title compound were obtained.

¹H-NMR (400 MHz, DMSO-d₆, δ/ppm): 11.00-10.00 (broad, 1H), 8.24-7.49 (m, 1H), 6.44 (s, 1H), 4.28 (q, 2H), 2.27 (s, 3H), 2.27-2.24 (m, 1H), 1.31 (t, 3H), 1.05 (br. d, 3H), 0.92-0.72 (m, 1H), 0.70-0.55 (m, 1H), 0.52-0.42 (m, 1H).

LC/MS (Method 1, ESIpos): Rₜ=2.04 min, m/z=283.11 [M+H]⁺.

### Example 302A

### 5-Methyl-3-[(S,2S)-2-methylcyclopropyl]thieno[2,3-d]pyrimidine-2,4(1H,3H)-dione

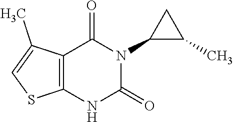 [SMILES: Cc1csc2[nH]c(=O)n([C@H]3C[C@@H]3C)c(=O)c12]

To a suspension of 293 g (1.04 mol) of the compound from Ex. 301A in 2.9 litres of ethanol were added 2.1 litres of a 21% solution of sodium ethoxide in ethanol. The resultant clear solution was stirred at 50° C. for 2.5 days. This was followed by cooling to RT and dilution with 2.9 litres of water. The mixture was brought to about pH 3 by adding 2.3 litres of 1 M hydrochloric acid, in the course of which the product precipitated out. The mixture was filtered with suction, washed with 2 litres of water and then dried over phosphorus pentoxide under reduced pressure. In this way, 205 g (83% of theory) of the title compound were obtained.

¹H-NMR (500 MHz, DMSO-d₆, δ/ppm): 11.90 (s, 1H), 6.63 (d, 1H), 2.33 (d, 3H), 2.17 (dt, 1H), 1.14 (d, 3H), 1.03-0.94 (m, 1H), 0.87-0.75 (m, 2H).

LC/MS (Method 1, ESIpos): Rₜ=1.28 min, m/z=237.07 [M+H]⁺.

### Example 303A

### 5-Methyl-3-[(1S,2S)-2-methylcyclopropyl]-2,4-dioxo-1,2,3,4-tetrahydrothieno[2,3-d]pyrimidine-6-carbaldehyde

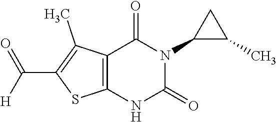 [SMILES: Cc1c(C=O)sc2[nH]c(=O)n([C@H]3C[C@@H]3C)c(=O)c12]

To an initially charged solution of 205 g (0.870 mol) of the compound from Ex. 302A in 2 litres (26.1 mol) of DMF at 60° C. were rapidly added 405 ml (4.35 mol) of phosphorus oxychloride over a period of 30 min. As a result of the exothermic reaction, the internal temperature rose to 80° C. The heating bath was lowered from the flask, and the rate of dropwise addition of the phosphorus oxychloride was adjusted such that the internal temperature stayed at about 80° C. After the dropwise addition had ended, the heating bath was raised back to the flask and the stirring was continued at 80° C. for a further 30 min. Subsequently, the mixture was cooled down to about 35° C. and 12.1 litres of water at 35° C. were poured in while stirring. The mixture was stirred at RT for about 2 h, during which the product gradually precipitated out. The solids were filtered off with suction, washed three times with 1.5 litres each time of water and then dried over phosphorus pentoxide under reduced pressure. 187 g (81% of theory) of the title compound were obtained.

¹H-NMR (400 MHz, DMSO-d₆, δ/ppm): 12.40 (s, 1H), 10.04 (s, 1H), 2.74 (s, 3H), 2.19 (dt, 1H), 1.14 (d, 3H), 1.07-0.95 (m, 1H), 0.90-0.75 (m, 2H).

LC/MS (Method 6, ESIpos): Rₜ=0.96 min, m/z=265 [M+H]⁺.

### Example 304A

### Ethyl 4-methyl-2-({[2-(trifluoromethyl)cyclopropyl]carbamoyl}amino)thiophene-3-carboxylate (trans racemate)

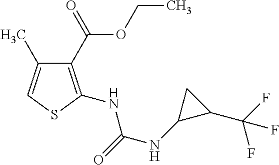 [SMILES: CCOC(=O)c1c(C)csc1NC(=O)NC1CC1C(F)(F)F]

To a solution of 1.0 g (5.63 mmol) of ethyl 2-amino-4-methylthiophene-3-carboxylate and 3.1 ml (22.5 mmol) of triethylamine in 18 ml of dichloromethane were added 1.14 g (7.03 mmol) of N,N′-carbonyldiimidazole (CDI), and the mixture was stirred at RT for 4 days. Then 1.0 g (6.19 mmol) of racemic trans-2-(trifluoromethyl)cyclopropanamine hydrochloride were added. After a further 2 h at RT, the reaction mixture was transferred to a separating funnel and washed successively with about 50 ml each time of water, 10% aqueous citric acid solution and saturated sodium chloride solution. After drying over anhydrous magnesium sulfate, the mixture was filtered and concentrated to dryness. The crude product that remained was purified by means of MPLC (Biotage Isolera One, SNAP KP-Sil cartridge, 50 g of silica gel, cyclohexane/ethyl acetate 5:1). After concentration of the product fractions and drying of the residue under high vacuum, 1.23 g (63% of theory, 97% purity) of the title compound were obtained.

¹H-NMR (400 MHz, DMSO-d₆, δ/ppm): 10.36 (br. s, 1H), 8.25 (br. s, 1H), 6.47 (s, 1H), 4.28 (q, 2H), 3.01-2.93 (m, 1H), 2.27 (s, 3H), 2.05-1.92 (m, 1H), 1.31 (t, 3H), 1.21-1.14 (m, 1H), 1.14-1.06 (m, 1H).

LC/MS (Method 1, ESIpos): Rₜ=2.09 min, m/z=337.08 [M+H]⁺.

### Example 305A

### 5-Methyl-3-[2-(trifluoromethyl)cyclopropyl]thieno[2,3-d]pyrimidine-2,4(1H,3H)-dione (trans racemate)

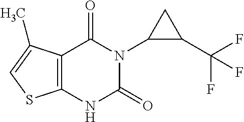 [SMILES: Cc1csc2[nH]c(=O)n(C3CC3C(F)(F)F)c(=O)c12]

1.23 g (3.55 mmol, 97% purity) of the compound from Ex. 304A were dissolved in 12 ml of ethanol, and 2.7 ml (7.10 mmol) of a 21% solution of sodium ethoxide in ethanol were added. The reaction mixture was stirred at RT for 3 h. Thereafter, the reaction mixture was acidified by adding 1 M hydrochloric acid (about pH 3). In the course of this, the product precipitated out. The product was filtered off with suction, washed to neutrality with water and dried under high vacuum. 1.04 g (97% of theory, 96% purity) of the title compound were obtained.

¹H-NMR (400 MHz, DMSO-d₆, δ/ppm): 12.04 (s, 1H), 6.67 (d, 1H), 2.93 (ddd, 1H), 2.34 (d, 3H), 2.24 (dtd, 1H), 1.50-1.42 (m, 1H), 1.34 (dt, 1H).

LC/MS (Method 1, ESIpos): Rₜ=1.51 min, m/z=291.04 [M+H]⁺.

### Example 306A

### 5-Methyl-2,4-dioxo-3-[2-(trifluoromethyl)cyclopropyl]-1,2,3,4-tetrahydrothieno[2,3-d]pyrimidine-6-carbaldehyde (trans racemate)

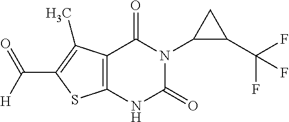 [SMILES: Cc1c(C=O)sc2[nH]c(=O)n(C3CC3C(F)(F)F)c(=O)c12]

To a solution of 1.04 g (3.58 mmol) of the compound from Ex. 305A in 3.9 ml (50.2 mmol) of DMF were cautiously added 4.0 ml (43.0 mmol) of phosphorus oxychloride. After the strongly exothermic reaction had abated, the mixture was stirred for another 30 min. Then the reaction mixture at RT was stirred cautiously into 400 ml of water. After stirring at RT for about 60 min, the precipitated product was filtered off with suction, washed to neutrality with water and dried under high vacuum. 1.03 g (90% of theory) of the title compound were obtained.

¹H-NMR (400 MHz, DMSO-d₆, δ/ppm): 12.53 (br. s, 1H), 10.06 (s, 1H), 2.93 (ddd, 1H), 2.75 (s, 3H), 2.31-2.18 (m, 1H), 1.49 (q, 1H), 1.40-1.30 (m, 1H).

LC/MS (Method 2, ESIpos): Rₜ=0.79 min, m/z=319 [M+H]⁺.

### Example 307A

### Ethyl 2-{[(2-ethylcyclopropyl)carbamoyl]amino}-4-methylthiophene-3-carboxylate (trans racemate)

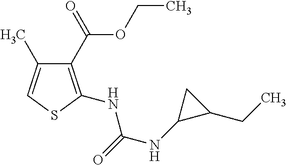 [SMILES: CCOC(=O)c1c(C)csc1NC(=O)NC1CC1CC]

Analogously to the process described in Ex. 304A, 1.39 g (7.48 mmol) of ethyl-2-amino-4-methylthiophene-3-carboxylate, 1.52 g (9.34 mmol) of N,N′-carbonyldiimidazole (CDI) and 1.0 g (8.22 mmol) of racemic trans-2-ethylcyclopropanamine hydrochloride were used to prepare 2.0 g (86% of theory, 96% purity) of the title compound.

¹H-NMR (400 MHz, DMSO-d₆, δ/ppm): 10.27 (broad, 1H), 7.91 (broad, 1H), 6.44 (s, 1H), 4.27 (q, 2H), 2.27 (s, 3H), 1.31 (t, 3H), 1.26-1.11 (m, 1H), 0.95 (t, 3H), 0.88-0.71 (m, 1H), 0.65-0.41 (m, 2H).

LC/MS (Method 1, ESIpos): Rₜ=2.15 min, m/z=297.13 [M+H]⁺.

### Example 308A

### 3-(2-Ethylcyclopropyl)-5-methylthieno[2,3-d]pyrimidine-2,4(1H,3H)-dione (trans racemate)

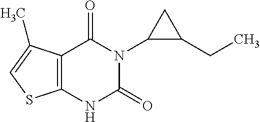 [SMILES: CCC1CC1n1c(=O)[nH]c2scc(C)c2c1=O]

Analogously to the method described in Ex. 305A, 2.0 g (6.55 mmol, 96% purity) of the compound from Ex. 307A were used to obtain 1.54 g (90% of theory, 96% purity) of the title compound. A difference here was that the reaction time was about 16 h.

¹H-NMR (400 MHz, DMSO-d₆, δ/ppm): 11.88 (s, 1H), 6.63 (d, 1H), 2.33 (d, 3H), 2.25 (dt, 1H), 1.69-1.55 (m, 1H), 1.28-1.14 (m, 1H), 1.07-0.93 (m, 1H), 0.99 (t, 3H), 0.84-0.75 (m, 2H).

LC/MS (Method 1, ESIpos): Rₜ=1.50 min, m/z=251.08 [M+H]⁺.

### Example 309A

### 3-(2-Ethylcyclopropyl)-5-methyl-2,4-dioxo-1,2,3,4-tetrahydrothieno[2,3-d]pyrimidine-6-carbaldehyde (trans racemate)

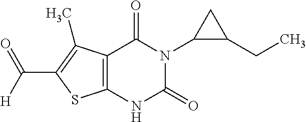 [SMILES: CCC1CC1n1c(=O)[nH]c2sc(C=O)c(C)c2c1=O]

Analogously to the method described in Ex. 306A, 1.54 g (5.91 mmol, 96% purity) of the compound from Ex. 308A, 6.6 ml (86.3 mmol) of DMF and 6.9 ml (74.0 mmol) of phosphorus oxychloride were used to obtain 1.64 g (98% of theory) of the title compound.

¹H-NMR (400 MHz, DMSO-d₆, δ/ppm): 12.38 (s, 1H), 10.04 (s, 1H), 2.74 (s, 3H), 2.30-2.22 (m, 1H), 1.68-1.50 (m, 1H), 1.30-1.15 (m, 1H), 1.09-0.92 (m, 1H), 1.00 (t, 3H), 0.86-0.76 (m, 2H).

LC/MS (Method 1, ESIpos): Rₜ=1.47 min, m/z=279.08 [M+H]⁺.

### Example 310A

### Ethyl 2-{[(2-methoxycyclopropyl)carbamoyl]amino}-4-methylthiophene-3-carboxylate (trans racemate)

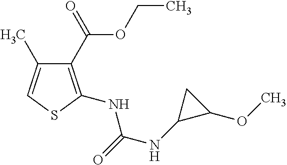

Analogously to the process described in Ex. 304A, 1.39 g (7.48 mmol) of ethyl-2-amino-4-methylthiophene-3-carboxylate, 1.52 g (9.34 mmol) of N,N′-carbonyldiimidazole (CDI) and 1.02 g (8.22 mmol) of racemic trans-2-methoxycyclopropanamine hydrochloride were used to prepare 1.90 g (85% of theory) of the title compound.

¹H-NMR (400 MHz, DMSO-d₆, δ/ppm): 10.37 (broad, 1H), 7.94 (broad, 1H), 6.44 (s, 1H), 4.28 (q, 2H), 3.29 (s, 3H), 3.23-3.16 (m, 1H), 2.66-2.59 (m, 1H), 2.27 (d, 3H), 1.31 (t, 3H), 0.99-0.83 (m, 1H), 0.57-0.43 (m, 1H).

LC/MS (Method 2, ESIpos): Rₜ=0.92 min, m/z=299 [M+H]⁺.

### Example 311A

### 3-(2-Methoxycyclopropyl)-5-methylthieno[2,3-d]pyrimidine-2,4(1H,3H)-dione (trans racemate)

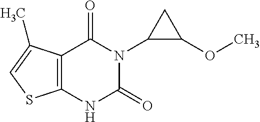 [SMILES: COC1CC1n1c(=O)[nH]c2scc(C)c2c1=O]

Analogously to the method described in Ex. 305A, 1.9 g (6.37 mmol) of the compound from Ex. 310A were used to obtain 1.55 g (96% of theory) of the title compound. A difference here was that the reaction time was about 16 h.

¹H-NMR (400 MHz, DMSO-d₆, δ/ppm): 11.94 (s, 1H), 6.66 (d, 1H), 3.45-3.36 (m, 1H), 3.23 (s, 3H), 2.59-2.52 (m, 1H), 2.33 (d, 3H), 1.28 (td, 1H), 0.95 (ddd, 1H).

LC/MS (Method 2, ESIpos): Rₜ=0.57 min, m/z=253 [M+H]⁺.

### Example 312A

### 3-(2-Methoxycyclopropyl)-5-methyl-2,4-dioxo-1,2,3,4-tetrahydrothieno[2,3-d]pyrimidine-6-carbaldehyde (trans racemate)

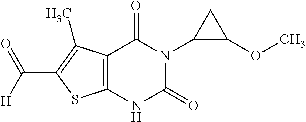 [SMILES: COC1CC1n1c(=O)[nH]c2sc(C=O)c(C)c2c1=O]

1.35 g (5.35 mmol) of the compound from Example 311A were dissolved in 21 ml of anhydrous DMF, and 5 ml (53.5 mmol) of phosphorus oxychloride were added gradually at 0° C. The mixture was stirred at 0° C. for about another 30 min, then the reaction mixture was stirred at 50° C. for 1 h and at 70° C. for a further hour. After cooling to RT, the reaction mixture was poured cautiously into 200 ml of water. The mixture was extracted with ethyl acetate, and the organic extract was washed with water and saturated aqueous sodium chloride solution. After drying over anhydrous magnesium sulfate, the mixture was filtered and concentrated. After the residue had been dried under high vacuum, 1.26 g (83% of theory) of the title compound were obtained.

¹H-NMR (400 MHz, DMSO-d₆, δ/ppm): 12.45 (s, 1H), 10.05 (s, 1H), 3.47-3.38 (m, 1H), 3.23 (s, 3H), 2.74 (s, 3H), 2.58 (dt, 1H), 1.30 (td, 1H), 0.94 (ddd, 1H).

LC/MS (Method 2, ESIpos): Rₜ=0.55 min, m/z=281 [M+H]⁺.

### Example 313A

### (2,2-Difluorocyclopentyl)methanol (racemate)

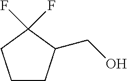

1.0 g (5.61 mmol) of racemic ethyl 2,2-difluorocyclopentanecarboxylate were dissolved in 20 ml of anhydrous THF, and 5.6 ml (5.61 mmol) of a 1 M solution of lithium aluminium hydride in THF were added dropwise at −78° C. After 30 min, the cooling bath was removed, and stirring was continued at RT. After 1 h at RT, saturated ammonium chloride solution was added cautiously and the mixture was extracted with ethyl acetate. The organic extract was dried over anhydrous magnesium sulfate, filtered and concentrated. 755 mg (96% of theory, 97% purity) of the title compound were obtained.

¹H-NMR (400 MHz, DMSO-d₆, δ/ppm): 4.61 (t, 1H), 3.57 (dt, 1H), 3.41-3.33 (m, 1H), 2.32-2.16 (m, 1H), 2.12-1.85 (m, 3H), 1.78-1.58 (m, 2H), 1.58-1.44 (m, 1H).

### Example 314A

### (3,3-Difluorocyclopentyl)methanol (racemate)

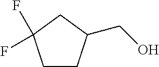

Analogously to the method described in Ex. 313A, 2.0 g (11.2 mmol) of racemic ethyl 3,3-difluorocyclopentanecarboxylate and 11.2 ml (11.2 mmol) of a 1 M solution of lithium aluminium hydride in THF were used to obtain 1.34 g (83% of theory, 95% purity) of the title compound.

¹H-NMR (400 MHz, DMSO-d₆, δ/ppm): 4.63 (t, 1H), 3.40-3.26 (m, 2H), 2.28-1.91 (m, 4H), 1.89-1.72 (m, 2H), 1.48 (dq, 1H).

### Example 315A

### (2,2-Difluorocyclobutyl)methyl 4-methylbenzenesulfonate (racemate)

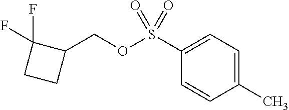 [SMILES: Cc1ccc(S(=O)(=O)OCC2CCC2(F)F)cc1]

To a solution of 1.0 g (7.94 mmol, 97% purity) of racemic (2,2-difluorocyclobutyl)methanol in 15 ml of dichloromethane were added 1.9 ml (23.8 mmol) of pyridine, and the mixture was cooled to 0° C. At this temperature, 1.67 g (8.74 mmol) of para-toluenesulfonyl chloride were added in portions. Then the reaction mixture was stirred at RT for about 18 h. Subsequently, it was diluted with dichloromethane, washed with water, dried over anhydrous magnesium sulfate, filtered and concentrated. 2.19 g (99% of theory) of the title compound were obtained.

¹H-NMR (400 MHz, DMSO-d₆, δ/ppm): 7.79 (d, 2H), 7.50 (d, 2H), 4.24-3.99 (m, 2H), 3.24-2.99 (m, 1H), 2.48-2.33 (m, 2H), 2.43 (s, 3H), 1.93-1.73 (m, 1H), 1.53-1.31 (m, 1H).

GC/MS (Method 9, EI): Rₜ=6.20 min, m/z=276 [M]⁺.

### Example 316A

### (3,3-Difluorocyclobutyl)methyl 4-methylbenzenesulfonate

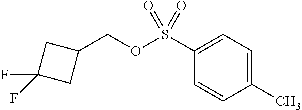 [SMILES: Cc1ccc(S(=O)(=O)OCC2CC(F)(F)C2)cc1]

To a solution of 4.0 g (31.1 mmol, 95% purity) of (3,3-difluorocyclobutyl)methanol in 60 ml of dichloromethane were added 7.6 ml (93.4 mmol) of pyridine, and the mixture was cooled to 0° C. At this temperature, 6.53 g (34.2 mmol) of para-toluenesulfonyl chloride were added in portions. Then the reaction mixture was stirred at RT for about 18 h. Subsequently, it was diluted with dichloromethane, washed with water, dried over anhydrous magnesium sulfate, filtered and concentrated. 8.65 g (98% of theory, 97% purity) of the title compound were obtained.

¹H-NMR (400 MHz, DMSO-d₆, δ/ppm): 7.80 (d, 2H), 7.50 (d, 2H), 4.09 (d, 2H), 2.67-2.56 (m, 2H), 2.43 (s, 3H), 2.37-2.21 (m, 3H).

GC/MS (Method 9, EI): Rₜ=6.13 min, m/z=276 [M]⁺.

### Example 317A

### (2,2-Difluorocyclopentyl)methyl 4-methylbenzenesulfonate (racemate)

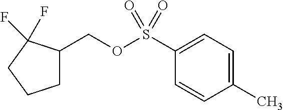 [SMILES: Cc1ccc(S(=O)(=O)OCC2CCCC2(F)F)cc1]

To a solution of 670 mg (4.43 mmol, 90% purity) of the compound from Ex. 313A in 10 ml of dichloromethane were added 1.1 ml (13.3 mmol) of pyridine, and the mixture was cooled to 0° C. At this temperature, 929 mg (4.87 mmol) of para-toluenesulfonyl chloride were added in portions. Then the reaction mixture was stirred at RT for about 18 h. Subsequently, it was diluted with dichloromethane, washed with water, dried over anhydrous magnesium sulfate, filtered and concentrated. The residue that remained was purified by means of MPLC (Biotage Isolera One, SNAP KP-Sil cartridge, 50 g of silica gel, cyclohexane/ethyl acetate 5:1). Concentration of the product fractions gave 1.23 g (95% of theory) of the title compound.

¹H-NMR (400 MHz, DMSO-d₆, δ/ppm): 7.79 (d, 2H), 7.49 (d, 2H), 4.11-3.99 (m, 2H), 2.62-2.53 (m, 1H), 2.43 (s, 3H), 2.19-1.82 (m, 3H), 1.74-1.55 (m, 2H), 1.46-1.32 (m, 1H).

### Example 318A

### (3,3-Difluorocyclopentyl)methyl 4-methylbenzenesulfonate (racemate)

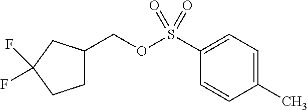 [SMILES: Cc1ccc(S(=O)(=O)OCC2CCC(F)(F)C2)cc1]

Analogously to the method described in Ex. 317A, 1.33 g (9.28 mmol, 95% purity) of the compound from Ex. 314A and 1.95 g (10.2 mmol) of para-toluenesulfonyl chloride were used to obtain 2.55 g (94% of theory) of the title compound.

¹H-NMR (400 MHz, DMSO-d₆, δ/ppm): 7.80 (d, 2H), 7.49 (d, 2H), 3.99 (d, 2H), 2.48-2.35 (m, 1H), 2.43 (s, 3H), 2.24-1.90 (m, 3H), 1.89-1.67 (m, 2H), 1.42 (dq, 1H).

GC/MS (Method 9, EI): Rₜ=6.66 min, m/z=290 [M]⁺.

### Example 319A

### 3-Cyclopropyl-1-ethyl-5-methyl-2,4-dioxo-1,2,3,4-tetrahydrothieno[2,3-d]pyrimidine-6-carbaldehyde

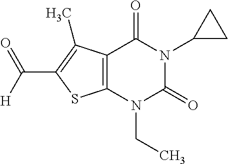 [SMILES: CCn1c(=O)n(C2CC2)c(=O)c2c(C)c(C=O)sc21]

Analogously to the process described in Ex. 63A, 130 mg (0.519 mmol) of the compound from Ex. 31A and 162 mg (1.04 mmol) of ethyl iodide were used to prepare 129 mg (89% of theory) of the title compound. The reaction time at RT here was 2.5 days, and the purification of the product by means of MPLC was dispensed with.

¹H-NMR (400 MHz, DMSO-d₆, δ/ppm): 10.08 (s, 1H), 3.93 (q, 2H), 2.77 (s, 3H), 2.65-2.57 (m, 1H), 1.24 (t, 3H), 1.07-0.97 (m, 2H), 0.76-0.66 (m, 2H).

LC/MS (Method 1, ESIpos): Rₜ=1.48 min, m/z=279.08 [M+H]⁺.

### Example 320A

### 3-Cyclopropyl-5-methyl-2,4-dioxo-1-propyl-1,2,3,4-tetrahydrothieno[2,3-d]pyrimidine-6-carbaldehyde

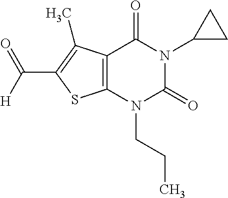 [SMILES: CCCn1c(=O)n(C2CC2)c(=O)c2c(C)c(C=O)sc21]

Analogously to the process described in Ex. 52A, 130 mg (0.519 mmol) of the compound from Ex. 31A and 177 mg (1.04 mmol) of propyl iodide were used to prepare 135 mg (88% of theory) of the title compound. The purification of the product by means of MPLC was dispensed with here.

¹H-NMR (400 MHz, DMSO-d₆, δ/ppm): 10.08 (s, 1H), 3.91-3.79 (m, 2H), 2.77 (s, 3H), 2.65-2.58 (m, 1H), 1.70 (sext, 2H), 1.06-0.97 (m, 2H), 0.92 (t, 3H), 0.75-0.66 (m, 2H).

LC/MS (Method 1, ESIpos): Rₜ=1.67 min, m/z=293.10 [M+H]⁺.

### Example 321A

### 3-Cyclopropyl-1-(2-fluoroethyl)-5-methyl-2,4-dioxo-1,2,3,4-tetrahydrothieno[2,3-d]pyrimidine-6-carbaldehyde

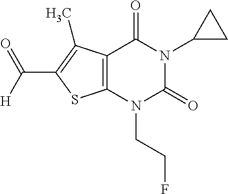 [SMILES: Cc1c(C=O)sc2c1c(=O)n(C1CC1)c(=O)n2CCF]

Analogously to the method described in Ex. 58A, 130 mg (0.519 mmol) of the compound from Ex. 31A and 181 mg (1.04 mmol) of 1-fluoro-2-iodoethane were used to prepare 84 mg (51% of theory, 94% purity) of the title compound. The reaction time at RT here was 2.5 days, and at 50° C. it was 2 h.

¹H-NMR (400 MHz, DMSO-d₆, δ/ppm): 10.08 (s, 1H), 4.74 (dt, 2H), 4.24 (dt, 2H), 2.77 (s, 3H), 2.66-2.58 (m, 1H), 1.06-0.97 (m, 2H), 0.77-0.68 (m, 2H).

LC/MS (Method 2, ESIpos): Rₜ=0.72 min, m/z=297 [M+H]⁺.

### Example 322A

### 3-Cyclopropyl-1-(4-fluorobutyl)-5-methyl-2,4-dioxo-1,2,3,4-tetrahydrothieno[2,3-d]pyrimidine-6-carbaldehyde

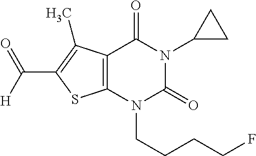 [SMILES: Cc1c(C=O)sc2c1c(=O)n(C1CC1)c(=O)n2CCCCF]

Analogously to the method described in Ex. 45A, 130 mg (0.519 mmol) of the compound from Ex. 31A and 161 mg (1.04 mmol) of 1-bromo-4-fluorobutane were used to prepare 129 mg (76% of theory) of the title compound. The reaction time at RT here was about 2.5 days; subsequently, the mixture was heated to 50° C. for about another 24 h.

¹H-NMR (400 MHz, DMSO-d₆, δ/ppm): 10.08 (s, 1H), 4.57-4.49 (m, 1H), 4.41 (t, 1H), 3.94 (t, 2H), 2.77 (s, 3H), 2.61 (tt, 1H), 1.85-1.61 (m, 4H), 1.08-0.95 (m, 2H), 0.75-0.65 (m, 2H).

LC/MS (Method 1, ESIpos): Rₜ=1.63 min, m/z=325.10 [M+H]⁺.

### Example 323A

### 3-Cyclopropyl-1-(2,2-difluoroethyl)-5-methyl-2,4-dioxo-1,2,3,4-tetrahydrothieno[2,3-d]pyrimidine-6-carbaldehyde

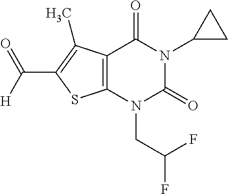 [SMILES: Cc1c(C=O)sc2c1c(=O)n(C1CC1)c(=O)n2CC(F)F]

150 mg (0.599 mmol) of the compound from Ex. 31A and 207 mg (1.50 mmol) of potassium carbonate were stirred in 4 ml of anhydrous DMF at RT for 15 min, before 219 μl (2.40 mmol) of 1,1-difluoro-2-iodoethane were added. Then the reaction mixture was stirred at 80° C. for 5 days. After cooling to RT, water was added and the mixture was extracted with ethyl acetate. The organic extract was washed successively with water and saturated sodium chloride solution, dried over anhydrous magnesium sulfate, filtered and concentrated. The product was purified by means of preparative HPLC (Method 11). After concentration of the product fractions and drying under high vacuum, this gave 94 mg (49% of theory) of the title compound.

¹H-NMR (400 MHz, DMSO-d₆, δ/ppm): 10.09 (s, 1H), 6.34 (tt, 1H), 4.38 (td, 2H), 2.77 (s, 3H), 2.69-2.59 (m, 1H), 1.08-0.97 (m, 2H), 0.77-0.68 (m, 2H).

LC/MS (Method 1, ESIpos): Rₜ=1.51 min, m/z=315.06 [M+H]⁺.

### Example 324A

### 3-Cyclopropyl-5-methyl-2,4-dioxo-1-(4,4,4-trifluorobutyl)-1,2,3,4-tetrahydrothieno[2,3-d]pyrimidine-6-carbaldehyde

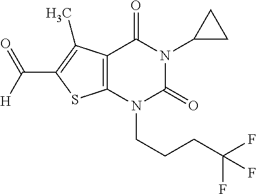 [SMILES: Cc1c(C=O)sc2c1c(=O)n(C1CC1)c(=O)n2CCCC(F)(F)F]

Analogously to the process described in Ex. 52A, 130 mg (0.519 mmol) of the compound from Ex. 31A and 371 mg (1.56 mmol) of 1,1,1-trifluoro-4-iodobutane were used to prepare 171 mg (88% of theory, 97% purity) of the title compound. Chromatographic purification of the product was dispensed with here.

¹H-NMR (400 MHz, DMSO-d₆, δ/ppm): 10.08 (s, 1H), 3.98 (t, 2H), 2.77 (s, 3H), 2.61 (tt, 1H), 2.51-2.34 (m, 2H, partially concealed by DMSO signal), 1.90 (dt, 2H), 1.07-0.95 (m, 2H), 0.76-0.64 (m, 2H).

LC/MS (Method 1, ESIpos): Rₜ=1.80 min, m/z=361.08 [M+H]⁺.

### Example 325A

### 3-Cyclopropyl-1-[(2,2-difluorocyclobutyl)methyl]-5-methyl-2,4-dioxo-1,2,3,4-tetrahydrothieno[2,3-d]pyrimidine-6-carbaldehyde (racemate)

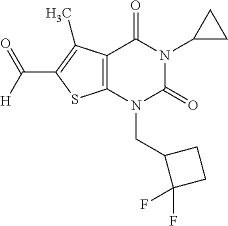 [SMILES: Cc1c(C=O)sc2c1c(=O)n(C1CC1)c(=O)n2CC1CCC1(F)F]

400 mg (1.60 mmol) of the compound from Ex. 31A and 552 mg (4.00 mmol) of potassium carbonate were stirred in 10 ml of anhydrous DMF at RT for 10 min, before 883 mg (3.20 mmol) of the compound from Ex. 315A were added. Then the reaction mixture was heated in a microwave oven (Biotage Initiator with dynamic control of irradiation power), first to 80° C. for 12 h and then to 100° C. for another 3 h. After cooling to RT, the reaction mixture was diluted with ethyl acetate and washed successively twice with water and once with saturated sodium chloride solution. After drying over anhydrous magnesium sulfate, filtration and concentration, the remaining residue was purified by means of MPLC (Biotage Isolera One, SNAP Ultra cartridge, 50 g of silica gel, cyclohexane/ethyl acetate 2:1). Concentration of the product fractions gave 364 mg (64% of theory) of the title compound.

¹H-NMR (400 MHz, DMSO-d₆, δ/ppm): 10.08 (s, 1H), 4.08 (dd, 2H), 3.37-3.21 (m, 2H, partially concealed by water signal), 2.77 (s, 3H), 2.62 (tt, 1H), 2.53-2.40 (m, 1H, partially concealed by DMSO signal), 2.02-1.87 (m, 1H), 1.64 (quin, 1H), 1.08-0.96 (m, 2H), 0.76-0.62 (m, 2H).

LC/MS (Method 1, ESIpos): Rₜ=1.74 min, m/z=355.09 [M+H]⁺.

### Example 326A

### 3-Cyclopropyl-1-[(2,2-difluorocyclobutyl)methyl]-5-methyl-2,4-dioxo-1,2,3,4-tetrahydrothieno[2,3-d]pyrimidine-6-carbaldehyde (enantiomer 1)

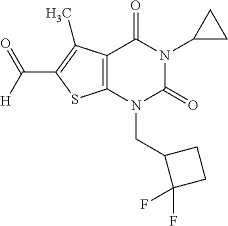 [SMILES: Cc1c(C=O)sc2c1c(=O)n(C1CC1)c(=O)n2CC1CCC1(F)F]

355 mg of the racemic compound from Ex. 325A were dissolved in 5 ml of acetonitrile/ethanol (1:1) and, in 100 portions, separated into the enantiomers by preparative HPLC on a chiral phase [column: Daicel Chiralpak IE, 5 μm, 250 mm×20 mm; eluent: n-heptane/ethanol 60:40; flow rate: 30 ml/min; temperature: 25° C.; detection: 220 nm]. The product fractions were each concentrated on a rotary evaporator, admixed with acetonitrile and water, and freeze-dried. 131 mg (90% of theory, 97.9% ee) of the title compound (enantiomer 1) and 117 mg (65% of theory, 96.3% ee) of enantiomer 2 (see Example 327A) were obtained.

¹H-NMR (400 MHz, DMSO-d₆, δ/ppm): 10.08 (s, 1H), 4.18-3.98 (m, 2H), 3.39-3.18 (m, 2H, partially concealed by water signal), 2.77 (s, 3H), 2.62 (tt, 1H), 2.55-2.40 (m, 1H, partially concealed by DMSO signal), 2.02-1.87 (m, 1H), 1.72-1.56 (m, 1H), 1.08-0.97 (m, 2H), 0.76-0.62 (m, 2H).

LC/MS (Method 1, ESIpos): Rₜ=1.76 min, m/z=355.09 [M+H]⁺.

Chiral analytical HPLC [column: Daicel Chiraltek IE, 3 μm, 100 mm×4.6 mm; eluent: isohexane/ethanol 1:1; flow rate: 1.0 ml/min; temperature: 30° C.; injection: 5 μl; DAD 220 nm]: Rₜ=4.64 min.

### Example 327A

### 3-Cyclopropyl-1-[(2,2-difluorocyclobuty)methyl]-5-methyl-2,4-dioxo-1,2,3,4-tetrahydrothieno[2,3-d]pyrimidine-6-carbaldehyde (enantiomer 2)

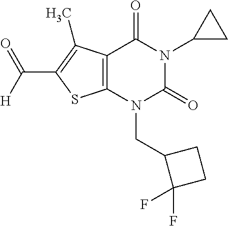 [SMILES: Cc1c(C=O)sc2c1c(=O)n(C1CC1)c(=O)n2CC1CCC1(F)F]

355 mg of the racemic compound from Ex. 325A were used to obtain, by means of the preparative HPLC on a chiral phase described in Ex. 326A, 117 mg (65% of theory, 96.3% ee) of the title compound (enantiomer 2) and 131 mg (90% of theory, 97.9% ee) of enantiomer 1 (see Ex. 326A).

¹H-NMR (400 MHz, DMSO-d₆, δ/ppm): 10.08 (s, 1H), 4.16-4.00 (m, 2H), 3.35-3.20 (m, 2H, partially concealed by water signal), 2.77 (s, 3H), 2.62 (tt, 1H), 2.55-2.40 (m, 1H, partially concealed by DMSO signal), 2.02-1.85 (m, 1H), 1.64 (quin, 1H), 1.08-0.96 (m, 2H), 0.75-0.63 (m, 2H).

LC/MS (Method 1, ESIpos): Rₜ=1.76 min, m/z=355.09 [M+H]⁺.

Chiral analytical HPLC [column: Daicel Chiraltek IE, 3 μm, 100 mm×4.6 mm; eluent: isohexane/ethanol 1:1; flow rate: 1.0 ml/min; temperature: 30° C.; injection: 5 μl; DAD 220 nm]: Rₜ=5.34 min.

### Example 328A

### 3-Cyclopropyl-1-[(3,3-difluorocyclobuty)methyl]-5-methyl-2,4-dioxo-1,2,3,4-tetrahydrothieno[2,3-d]pyrimidine-6-carbaldehyde

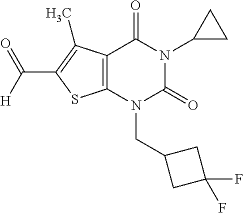 [SMILES: Cc1c(C=O)sc2c1c(=O)n(C1CC1)c(=O)n2CC1CC(F)(F)C1]

300 mg (1.20 mmol) of the compound from Ex. 31A and 415 mg (3.00 mmol) of potassium carbonate were stirred in 12 ml of anhydrous DMF at RT for 10 min, before 662 mg (2.40 mmol) of the compound from Ex. 316A were added. Then the reaction mixture was heated in a microwave oven (Biotage Initiator with dynamic control of irradiation power) to 80° C. for 16 h. After cooling to RT, the reaction mixture was diluted with ethyl acetate and washed successively twice with water and once with saturated sodium chloride solution. After drying over anhydrous magnesium sulfate, filtration and concentration, the remaining residue was purified by means of MPLC (Biotage Isolera One, SNAP Ultra cartridge, 50 g of silica gel, cyclohexane/ethyl acetate 2:1). Concentration of the product fractions gave 248 mg (58% of theory) of the title compound.

¹H-NMR (500 MHz, DMSO-d₆, δ/ppm): 10.08 (s, 1H), 4.09 (d, 2H), 2.77 (s, 3H), 2.73-2.57 (m, 4H), 2.57-2.46 (m, 2H, partially concealed by DMSO signal), 1.06-0.96 (m, 2H), 0.74-0.64 (m, 2H).

LC/MS (Method 6, ESIpos): Rₜ=1.22 min, m/z=355 [M+H]⁺.

### Example 329A

### 3-Cyclopropyl-1-[(2,2-difluorocyclopentyl)methyl]-5-methyl-2,4-dioxo-1,2,3,4-tetrahydrothieno[2,3-d]pyrimidine-6-carbaldehyde (racemate)

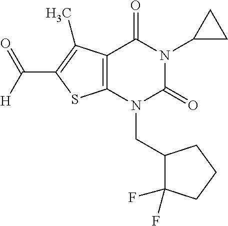 [SMILES: Cc1c(C=O)sc2c1c(=O)n(C1CC1)c(=O)n2CC1CCCC1(F)F]

300 mg (1.20 mmol) of the compound from Ex. 31A and 415 mg (3.00 mmol) of potassium carbonate were stirred in 12 ml of anhydrous DMF at RT for 10 min, before 696 mg (2.40 mmol) of the compound from Ex. 317A were added. Then the reaction mixture was heated in a microwave oven (Biotage Initiator with dynamic control of irradiation power), first to 80° C. for 20 h and subsequently to 100° C. for another 5 h. After cooling to RT, the reaction mixture was diluted with ethyl acetate and washed successively twice with water and once with saturated sodium chloride solution. After drying over anhydrous magnesium sulfate, filtration and concentration, the remaining residue was purified by means of MPLC (Biotage Isolera One, SNAP Ultra cartridge, 50 g of silica gel, cyclohexane/ethyl acetate 2:1). Concentration of the product fractions gave 270 mg (61% of theory) of the title compound.

¹H-NMR (400 MHz, DMSO-d₆, δ/ppm): 10.08 (s, 1H), 4.11-3.91 (m, 2H), 2.84-2.66 (m, 1H), 2.77 (s, 3H), 2.66-2.58 (m, 1H), 2.23-2.02 (m, 2H), 2.01-1.88 (m, 1H), 1.84-1.51 (m, 3H), 1.09-0.95 (m, 2H), 0.79-0.61 (m, 2H).

LC/MS (Method 1, ESIpos): Rₜ=1.87 min, m/z=369.11 [M+H]⁺.

### Example 330A

### 3-Cyclopropyl-1-[(2,2-difluorocyclopentyl)methyl]-5-methyl-2,4-dioxo-1,2,3,4-tetrahydrothieno[2,3-d]pyrimidine-6-carbaldehyde (enantiomer 1)

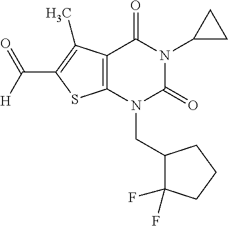 [SMILES: Cc1c(C=O)sc2c1c(=O)n(C1CC1)c(=O)n2CC1CCCC1(F)F]

268 mg of the racemic compound from Ex. 329A were dissolved in 8 ml of acetonitrile and, in 18 portions, separated into the enantiomers by preparative HPLC on a chiral phase [column: Daicel Chiralpak AD-H, 5 μm, 250 mm×30 mm; eluent: n-heptane/ethanol 30:70; flow rate: 40 ml/min; temperature: 25° C.; detection: 220 nm]. The product fractions were each concentrated on a rotary evaporator, admixed with acetonitrile and water, and freeze-dried. 120 mg (89% of theory, >99% ee) of the title compound (enantiomer 1) and 118 mg (88% of theory, >99% ee) of enantiomer 2 (see Example 331A) were obtained.

¹H-NMR (400 MHz, DMSO-d₆, δ/ppm): 10.08 (s, 1H), 4.10-3.91 (m, 2H), 2.83-2.66 (m, 1H), 2.77 (s, 3H), 2.66-2.58 (m, 1H), 2.23-2.02 (m, 2H), 2.01-1.88 (m, 1H), 1.84-1.50 (m, 3H), 1.09-0.94 (m, 2H), 0.77-0.59 (m, 2H).

LC/MS (Method 2, ESIpos): Rₜ=0.97 min, m/z=369 [M+H]⁺.

Chiral analytical HPLC [column: Daicel Chiraltek AD-3, 3 μm 100 mm×4.6 mm; eluent: n-heptane/ethanol 1:1; flow rate: 3.0 ml/min; temperature: 30° C.; injection: 5 μl; DAD 220 nm]: Rₜ=3.61 min.

### Example 331A

### 3-Cyclopropyl-1-[(2,2-difluorocyclopentyl)methyl]-5-methyl-2,4-dioxo-1,2,3,4-tetrahydrothieno[2,3-d]pyrimidine-6-carbaldehyde (enantiomer 2)

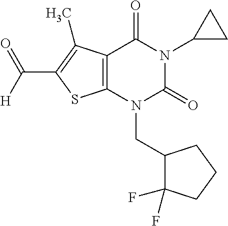 [SMILES: Cc1c(C=O)sc2c1c(=O)n(C1CC1)c(=O)n2CC1CCCC1(F)F]

268 mg of the racemic compound from Ex. 329A were used to obtain, by means of the preparative HPLC on a chiral phase described in Ex. 330A, 118 mg (88% of theory, >99% ee) of the title compound (enantiomer 2) and 120 mg (89% of theory, >99% ee) of enantiomer 1 (see Ex. 330A).

¹H-NMR (400 MHz, DMSO-d₆, δ/ppm): 10.08 (s, 1H), 4.10-3.91 (m, 2H), 2.82-2.66 (m, 1H), 2.77 (s, 3H), 2.66-2.58 (m, 1H), 2.24-2.02 (m, 2H), 2.01-1.88 (m, 1H), 1.84-1.48 (m, 3H), 1.12-0.94 (m, 2H), 0.77-0.60 (m, 2H).

LC/MS (Method 2, ESIpos): Rₜ=0.96 min, m/z=369 [M+H]⁺.

Chiral analytical HPLC [column: Daicel Chiraltek AD-3, 3 μm 100 mm×4.6 mm; eluent: n-heptane/ethanol 1:1; flow rate: 3.0 ml/min; temperature: 30° C.; injection: 5 μl; DAD 220 nm]: Rₜ=8.94 min.

### Example 332A

### 3-Cyclopropyl-1-[(3,3-difluorocyclopentyl)methyl]-5-methyl-2,4-dioxo-1,2,3,4-tetrahydrothieno[2,3-d]pyrimidine-6-carbaldehyde (racemate)

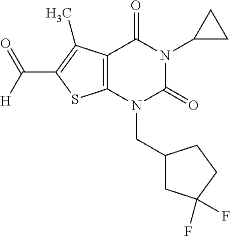 [SMILES: Cc1c(C=O)sc2c1c(=O)n(C1CC1)c(=O)n2CC1CCC(F)(F)C1]

Analogously to the method described in Ex. 328A, 300 mg (1.20 mmol) of the compound from Ex. 31A and 696 mg (2.40 mmol) of the compound from Ex. 318A were used to obtain 295 mg (66% of theory) of the title compound.

¹H-NMR (400 MHz, DMSO-d₆, δ/ppm): 10.08 (s, 1H), 4.05-3.81 (m, 2H), 2.77 (s, 3H), 2.69-2.56 (m, 2H), 2.38-1.83 (m, 5H), 1.60 (dq, 1H), 1.08-0.96 (m, 2H), 0.77-0.63 (m, 2H).

LC/MS (Method 6, ESIpos): Rₜ=1.27 min, m/z=369 [M+H]⁺.

### Example 333A

### 4-(3-Cyclopropyl-6-formyl-5-methyl-2,4-dioxo-3,4-dihydrothieno[2,3-d]pyrimidin-1(2H)-yl)butanenitrile

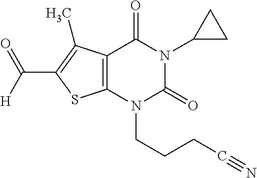 [SMILES: Cc1c(C=O)sc2c1c(=O)n(C1CC1)c(=O)n2CCCC#N]

Analogously to the method described in Ex. 66A, 130 mg (0.519 mmol) of the compound from Ex. 31A and 154 mg (1.04 mmol) of 4-bromobutyronitrile were used to prepare 143 mg (86% of theory) of the title compound. Chromatography was dispensed with here.

¹H-NMR (400 MHz, DMSO-d₆, δ/ppm): 10.08 (s, 1H), 3.99 (t, 2H), 2.77 (s, 3H), 2.67-2.56 (m, 3H), 1.99 (quin, 2H), 1.08-0.96 (m, 2H), 0.76-0.66 (m, 2H).

LC/MS (Method 1, ESIpos): Rₜ=1.35 min, m/z=318.09 [M+H]⁺.

### Example 334A

### 3-Cyclopropyl-5-methyl-2,4-dioxo-1-{2-[(trifluoromethyl)sulfanyl]ethyl}-1,2,3,4-tetrahydrothieno[2,3-d]pyrimidine-6-carbaldehyde

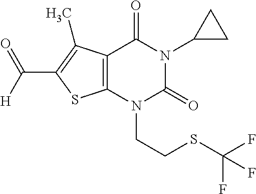 [SMILES: Cc1c(C=O)sc2c1c(=O)n(C1CC1)c(=O)n2CCSC(F)(F)F]

Analogously to the method described in Ex. 69A, 130 mg (0.519 mmol) of the compound from Ex. 31A and 217 mg (1.04 mmol) of 1-bromo-2-[(trifluoromethyl)sulfanyl]ethane were used to prepare 172 mg (85% of theory, 98% purity) of the title compound. A difference here was that the mixture was stirred at 60° C. for 3 h.

¹H-NMR (400 MHz, DMSO-d₆, δ/ppm): 10.08 (s, 1H), 4.17 (t, 2H), 3.34 (t, 2H), 2.77 (s, 3H), 2.66-2.57 (m, 1H), 1.08-0.97 (m, 2H), 0.75-0.63 (m, 2H).

LC/MS (Method 2, ESIpos): Rₜ=0.98 min, m/z=379 [M+H]⁺.

### Example 335A

### 1-[(3,3-Difluorocyclobutyl)methyl]-5-methyl-3-(1-methylcyclopropyl)-2,4-dioxo-1,2,3,4-tetrahydrothieno[2,3-d]pyrimidine-6-carbaldehyde

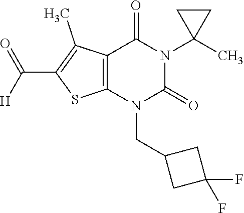 [SMILES: Cc1c(C=O)sc2c1c(=O)n(C1(C)CC1)c(=O)n2CC1CC(F)(F)C1]

400 mg (1.51 mmol) of the compound from Ex. 32A and 314 mg (2.27 mmol) of potassium carbonate were stirred in 15 ml of anhydrous DMF at RT for 10 min, before 627 mg (2.27 mmol) of the compound from Ex. 316A were added. Then the reaction mixture was heated in a microwave oven (Biotage Initiator with dynamic control of irradiation power) to 80° C. for 15 h. After cooling to RT, the reaction mixture was diluted with ethyl acetate and washed successively twice with water and once with saturated sodium chloride solution. After drying over anhydrous magnesium sulfate, filtration and concentration, the remaining residue was purified by means of MPLC (Biotage Isolera One, SNAP Ultra cartridge, 50 g of silica gel, cyclohexane/ethyl acetate 2:1). Concentration of the product fractions gave 380 mg (68% of theory) of the title compound.

¹H-NMR (400 MHz, DMSO-d₆, δ/ppm): 10.08 (s, 1H), 4.25-3.90 (m, 2H), 2.77 (s, 3H), 2.73-2.58 (m, 3H), 1.34 (s, 3H), 1.01-0.74 (m, 4H).

LC/MS (Method 1, ESIpos): Rₜ=1.89 min, m/z=369.11 [M+H]⁺.

### Example 336A

### 1,5-Dimethyl-3-(2-methylcyclopropyl)-2,4-dioxo-1,2,3,4-tetrahydrothieno[2,3-d]pyrimidine-6-carbaldehyde (trans racemate)

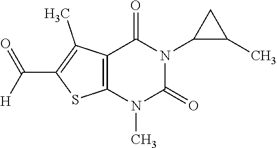 [SMILES: Cc1c(C=O)sc2c1c(=O)n(C1CC1C)c(=O)n2C]

200 mg (0.8 mmol) of the compound from Ex. 34A and 261 mg (1.9 mmol) of potassium carbonate were initially charged in 2.9 ml of anhydrous DMF, 141 μl (2.3 mmol) of methyl iodide were added and the mixture was stirred at RT for 1 h. Then 3 ml of water were added to the reaction mixture, which was stirred at RT for 10 min. The product which precipitated out was filtered off with suction, washed with water and then dried under high vacuum. 160 mg (76% of theory) of the title compound were obtained.

¹H-NMR (400 MHz, DMSO-d₆, δ/ppm): 10.07 (s, 1H), 3.43 (s, 3H), 2.77 (s, 3H), 2.25 (m, 1H), 1.16 (d, 3H), 1.07-0.96 (m, 1H), 0.90-0.80 (m, 2H).

LC/MS (Method 1, ESIpos): Rₜ=1.52 min, m/z=279.08 [M+H]⁺.

### Example 337A

### 1-Ethyl-5-methyl-3-(2-methylcyclopropyl)-2,4-dioxo-1,2,3,4-tetrahydrothieno[2,3-d]pyrimidine-6-carbaldehyde (trans racemate)

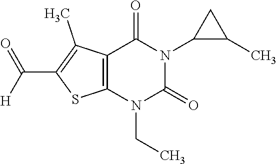 [SMILES: CCn1c(=O)n(C2CC2C)c(=O)c2c(C)c(C=O)sc21]

200 mg (0.8 mmol) of the compound from Ex. 34A and 261 mg (1.9 mmol) of potassium carbonate were initially charged in 2.9 ml of anhydrous DMF, 354 mg (2.3 mmol) of ethyl iodide were added and the mixture was heated to 80° C. in a microwave (Biotage Initiator) for 1 h. Then water was added to the reaction mixture, which was stirred at RT for 10 min. The product which precipitated out was filtered off with suction, washed with water and then dried under high vacuum. 241 mg (99% of theory, 91% purity) of the title compound were obtained.

¹H-NMR (400 MHz, DMSO-d₆, δ/ppm): 10.08 (s, 1H), 3.93 (q, 2H), 2.77 (s, 3H), 2.26 (m, 1H), 1.24 (t, 3H), 1.15 (d, 3H), 1.07-0.96 (m, 1H), 0.90-0.80 (m, 2H).

LC/MS (Method 2, ESIpos): Rₜ=0.88 min, m/z=293.2 [M+H]⁺.

### Example 338A

### 5-Methyl-3-(2-methylcyclopropyl)-2,4-dioxo-1-propyl-1,2,3,4-tetrahydrothieno[2,3-d]pyrimidine-6-carbaldehyde (trans racemate)

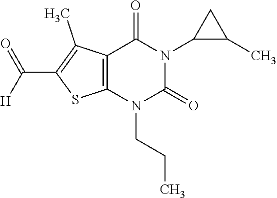 [SMILES: CCCn1c(=O)n(C2CC2C)c(=O)c2c(C)c(C=O)sc21]

200 mg (0.8 mmol) of the compound from Ex. 34A and 261 mg (1.9 mmol) of potassium carbonate were initially charged in 2.9 ml of anhydrous DMF, 221 μl (2.3 mmol) of 1-iodopropane were added and the mixture was stirred at RT for 15 h. Then 3 ml of water were added to the reaction mixture, which was stirred at RT for 10 min. The liquor was decanted off from the tacky residue, and the residue was again admixed with water and stirred. The liquor was decanted off again and the residue that remained was dried under high vacuum. 210 mg (83% of theory, 92% purity) of the title compound were obtained.

¹H-NMR (400 MHz, DMSO-d₆, δ/ppm): 10.07 (s, 1H), 3.86 (m, 2H), 2.77 (s, 3H), 2.26 (m, 1H), 1.15 (d, 3H), 1.07-0.96 (m, 1H), 0.92 (t, 3H), 0.88-0.80 (m, 2H).

LC/MS (Method 2, ESIpos): Rₜ=0.97 min, m/z=307.1 [M+H]⁺.

### Example 339A

### 1-Butyl-5-methyl-3-[(1S,2S)-2-methylcyclopropyl]-2,4-dioxo-1,2,3,4-tetrahydrothieno[2,3-d]pyrimidine-6-carbaldehyde

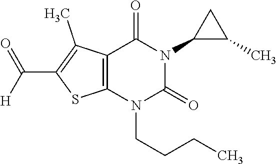 [SMILES: CCCCn1c(=O)n([C@H]2C[C@@H]2C)c(=O)c2c(C)c(C=O)sc21]

300 mg (1.14 mmol) of the compound from Ex. 303A were dissolved in 2.9 ml of anhydrous DMF, 555 mg (1.70 mmol) of caesium carbonate were added, and the mixture was stirred at RT for 10 min. Then 388 μl (3.41 mmol) of 1-iodobutane were added. After the reaction mixture had been stirred in a microwave oven (Biotage Initiator with dynamic control of irradiation power) at 100° C. for 60 min, it was poured onto water and acidified by addition of 1 M hydrochloric acid. In the course of this, the product precipitated out. After stirring at RT for 1 h, the solids were filtered off with suction, washed with a little water and dried under high vacuum. 345 mg (92% of theory) of the title compound were obtained.

¹H-NMR (500 MHz, DMSO-d₆, δ/ppm): 10.07 (s, 1H), 3.95-3.82 (m, 2H), 2.77 (s, 3H), 2.29-2.22 (m, 1H), 1.65 (quin, 2H), 1.41-1.30 (m, 2H), 1.15 (d, 3H), 1.06-0.96 (m, 1H), 0.91 (t, 3H), 0.88-0.81 (m, 2H).

LC/MS (Method 1, ESIpos): Rₜ=2.06 min, m/z=321.13 [M+H]⁺.

### Example 340A

### 1-(2-Fluoroethyl)-5-methyl-3-(2-methylcyclopropyl)-2,4-dioxo-1,2,3,4-tetrahydrothieno[2,3-d]pyrimidine-6-carbaldehyde (trans racemate)

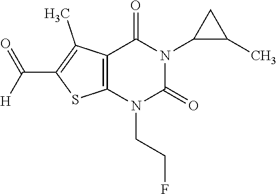 [SMILES: Cc1c(C=O)sc2c1c(=O)n(C1CC1C)c(=O)n2CCF]

200 mg (0.8 mmol) of the compound from Ex. 34A and 261 mg (1.9 mmol) of potassium carbonate were initially charged in 2.9 ml of anhydrous DMF, 403 mg (2.31 mmol) of 1-fluoro-2-iodoethane were added and the mixture was heated to 80° C. in a microwave (Biotage Initiator) for 1 h. Water was then added and the reaction mixture was extracted with ethyl acetate. The organic phase was washed with water and saturated aqueous sodium chloride solution, dried over sodium sulfate and filtered, and the filtrate was concentrated to dryness on a rotary evaporator. The residue that remained was converted in subsequent reactions without further purification. 212 mg (90% of theory, 90% purity) of the title compound were obtained.

¹H-NMR (400 MHz, DMSO-d₆, δ/ppm): 10.08 (s, 1H), 4.73 (dt, 2H), 4.23 (dm, 2H), 2.77 (s, 3H), 2.26 (m, 1H), 1.15 (d, 3H), 1.09-0.98 (m, 1H), 0.91-0.82 (m, 2H).

LC/MS (Method 2, ESIpos): Rₜ=0.83 min, m/z=311.2 [M+H]⁺.

### Example 341A

### 1-(2,2-Difluoroethyl)-5-methyl-3-(2-methylcyclopropyl)-2,4-dioxo-1,2,3,4-tetrahydrothieno[2,3-d]pyrimidine-6-carbaldehyde (trans racemate)

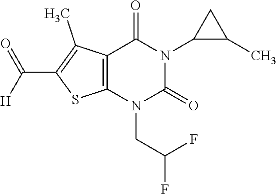 [SMILES: Cc1c(C=O)sc2c1c(=O)n(C1CC1C)c(=O)n2CC(F)F]

200 mg (0.8 mmol) of the compound from Ex. 34A and 261 mg (1.9 mmol) of potassium carbonate were initially charged in 2.9 ml of anhydrous DMF, 435 mg (2.26 mmol) of 1,1-difluoro-2-iodoethane were added and the mixture was first heated to 80° C. in a microwave (Biotage Initiator) for 1 h. Subsequently, the mixture was heated to 100° C. for a further 3 h and then to 120° C. for another 1 h. The reaction mixture was then separated directly into its components by means of preparative HPLC (Method 16). 169 mg (68% of theory) of the title compound were obtained.

¹H-NMR (400 MHz, DMSO-d₆, δ/ppm): 10.09 (s, 1H), 6.34 (tt, 1H), 4.38 (tt, 2H), 2.77 (s, 3H), 2.28 (m, 1H), 1.15 (d, 3H), 1.09-0.98 (m, 1H), 0.91-0.82 (m, 2H).

### Example 342A

### 5-Methyl-3-(2-methylcyclopropyl)-2,4-dioxo-1-(2,2,2-trifluoroethyl)-1,2,3,4-tetrahydrothieno[2,3-d]pyrimidine-6-carbaldehyde (trans racemate)

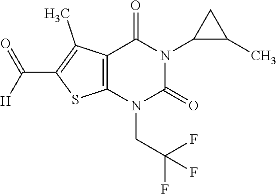 [SMILES: Cc1c(C=O)sc2c1c(=O)n(C1CC1C)c(=O)n2CC(F)(F)F]

200 mg (0.8 mmol) of the compound from Ex. 34A and 261 mg (1.9 mmol) of potassium carbonate were initially charged in 2.9 ml of anhydrous DMF, 476 mg (2.27 mmol) of 1,1,1-trifluoro-2-iodoethane were added and the mixture was heated to 140° C. in a microwave (Biotage Initiator) for 3 h. The reaction mixture was then filtered and the filtrate was separated into its components by means of preparative HPLC (Method 16). 105 mg (39% of theory) of the title compound were obtained.

¹H-NMR (400 MHz, DMSO-d₆, δ/ppm): 10.10 (s, 1H), 4.91 (m, 2H), 2.77 (s, 3H), 2.31 (m, 1H), 1.16 (d, 3H), 1.09-0.98 (m, 1H), 0.89-0.83 (m, 2H).

LC/MS (Method 1, ESIpos): Rₜ=1.84 min, m/z=347.07 [M+H]⁺.

### Example 343A

### 5-Methyl-3-(2-methylcyclopropyl)-2,4-dioxo-1-(4,4,4-trifluorobutyl)-1,2,3,4-tetrahydrothieno[2,3-d]pyrimidine-6-carbaldehyde (trans racemate)

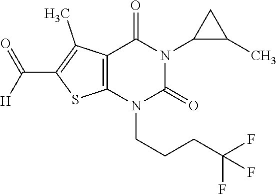 [SMILES: Cc1c(C=O)sc2c1c(=O)n(C1CC1C)c(=O)n2CCCC(F)(F)F]

200 mg (0.8 mmol) of the compound from Ex. 34A and 261 mg (1.9 mmol) of potassium carbonate were initially charged in 2.9 ml of anhydrous DMF, 540 mg (2.27 mmol) of 1,1,1-trifluoro-4-iodobutane were added and the mixture was heated to 80° C. in a microwave (Biotage Initiator) for 1 h. Then water was added to the reaction mixture, which was stirred at RT for 10 min. The product which precipitated out was filtered off with suction, washed with water and then dried under high vacuum. 250 mg (81% of theory, 92% purity) of the title compound were obtained.

¹H-NMR (400 MHz, DMSO-d₆, δ/ppm): 10.08 (s, 1H), 3.99 (m, 2H), 2.77 (s, 3H), 2.44 (m, 2H), 2.25 (m, 1H), 1.89 (m, 2H), 1.15 (d, 3H), 1.07-0.97 (m, 1H), 0.90-0.80 (m, 2H).

LC/MS (Method 1, ESIpos): Rₜ=1.99 min, m/z=375.1 [M+H]⁺.

### Example 344A

### 5-Methyl-3-(2-methylcyclopropyl)-2,4-dioxo-1-(3,3,4,4-tetrafluorobutyl)-1,2,3,4-tetrahydrothieno[2,3-d]pyrimidine-6-carbaldehyde (trans racemate)

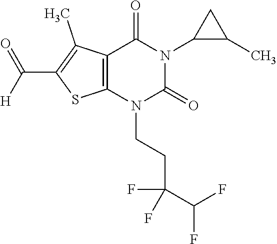 [SMILES: Cc1c(C=O)sc2c1c(=O)n(C1CC1C)c(=O)n2CCC(F)(F)C(F)F]

200 mg (0.8 mmol) of the compound from Ex. 34A and 261 mg (1.9 mmol) of potassium carbonate were initially charged in 2.9 ml of anhydrous DMF, 499 mg (2.27 mmol) of 1-bromo-3,3,4,4-tetrafluorobutane were added and the mixture was stirred at RT overnight. Then the reaction mixture was stirred once again at 35° C. for 16 h and finally at 60° C. for 6 h. Then 5 ml of water were added to the reaction mixture, which was extracted with ethyl acetate. The organic phase was dried with sodium sulfate, filtered and concentrated on a rotary evaporator. The residue that remained was chromatographed using a silica gel cartridge (Biotage, 10 g SNAP Ultra, eluent: cyclohexane/0-100% ethyl acetate). In this way, 193 mg (59% of theory, 90% purity) of the title compound were obtained.

¹H-NMR (400 MHz, DMSO-d₆, δ/ppm): 10.09 (s, 1H), 6.55 (tt, 1H), 4.13 (m, 2H), 2.78 (s, 3H), 2.57-2.44 (m, 2H), 2.26 (m, 1H), 1.15 (d, 3H), 1.07-0.98 (m, 1H), 0.89-0.82 (m, 2H).

LC/MS (Method 1, ESIpos): Rₜ=1.93 min, m/z=393.1 [M+H]⁺.

### Example 345A

### 1-(Cyclobutylmethyl)-5-methyl-3-[(1S,2S)-2-methylcyclopropyl]-2,4-dioxo-1,2,3,4-tetrahydrothieno[2,3-d]pyrimidine-6-carbaldehyde

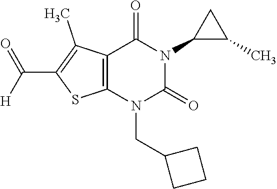 [SMILES: Cc1c(C=O)sc2c1c(=O)n([C@H]1C[C@@H]1C)c(=O)n2CC1CCC1]

To a solution of 300 mg (1.14 mmol) of the compound from Ex. 303A in 5 ml of anhydrous DMF were added 392 mg (2.84 mmol) of potassium carbonate, and the mixture was stirred at RT for 15 min. Then 507 mg (3.41 mmol) of (bromomethyl)cyclobutane were added, and the reaction mixture was stirred at 50° C. for about 16 h. After cooling to RT, the mixture was diluted with 200 ml of ethyl acetate and washed successively with water and saturated sodium chloride solution. After drying over anhydrous magnesium sulfate, the mixture was filtered and the concentrated. The residue that remained was purified by means of MPLC (Biotage Isolera One, SNAP Ultra cartridge, 25 g of silica gel, cyclohexane/ethyl acetate gradient). After concentration of the product fraction and drying under high vacuum, 335 mg (89% of theory) of the title compound were obtained.

¹H-NMR (500 MHz, DMSO-d₆, δ/ppm): 10.07 (s, 1H), 4.02-3.89 (m, 2H), 2.82-2.70 (m, 1H), 2.76 (s, 3H), 2.26 (dt, 1H), 2.03-1.93 (m, 2H), 1.88-1.74 (m, 4H), 1.15 (d, 3H), 1.06-0.95 (m, 1H), 0.89-0.80 (m, 2H).

LC/MS (Method 1, ESIpos): Rₜ=2.09 min, m/z=333.13 [M+H]⁺.

### Example 346A

### 1-[(2,2-Difluorocyclopropyl)methyl]-5-methyl-3-[(1S,2S)-2-methylcyclopropyl]-2,4-dioxo-1,2,3,4-tetrahydrothieno[2,3-d]pyrimidine-6-carbaldehyde (diastereomer mixture)

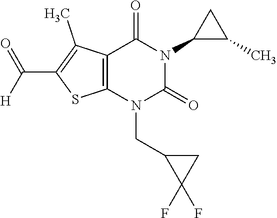 [SMILES: Cc1c(C=O)sc2c1c(=O)n([C@H]1C[C@@H]1C)c(=O)n2CC1CC1(F)F]

258 mg (0.975 mmol) of the compound from Ex. 303A were dissolved in 5 ml of anhydrous DMF, 337 mg (2.44 mmol) of potassium carbonate were added, and the mixture was stirred at RT for 15 min. Then 250 mg (1.46 mmol) of racemic 2-(bromomethyl)-1,1-difluorocyclopropane were added. After the reaction mixture had been stirred at 50° C. for about 16 h, it was poured onto water. In the course of this, the product precipitated out. After stirring at RT for 30 min, the solids were filtered off with suction, washed with a little water and dried under high vacuum. 328 mg (91% of theory, 96% purity) of the title compound were obtained.

¹H-NMR (500 MHz, DMSO-d₆, δ/ppm): 10.09 (s, 1H), 4.23-4.11 (m, 1H), 3.95 (ddd, 1H), 2.78 (s, 3H), 2.27 (dt, 1H), 2.25-2.15 (m, 1H), 1.76-1.64 (m, 1H), 1.49 (dtd, 1H), 1.16 (d, 3H), 1.07-0.98 (m, 1H), 0.90-0.81 (m, 2H).

LC/MS (Method 2, ESIpos): Rₜ=1.00 min, m/z=355 [M+H]⁺.

### Example 347A

### 1-[(3,3-Difluorocyclobutyl)methyl]-5-methyl-3-(2-methylcyclopropyl)-2,4-dioxo-1,2,3,4-tetrahydrothieno[2,3-d]pyrimidine-6-carbaldehyde (trans racemate)

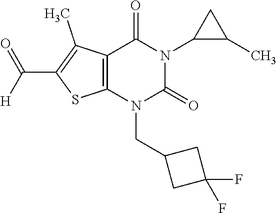 [SMILES: Cc1c(C=O)sc2c1c(=O)n(C1CC1C)c(=O)n2CC1CC(F)(F)C1]

319 mg (1.21 mmol) of the compound from Ex. 34A and 250 mg (1.81 mmol) of potassium carbonate were initially charged in 4.5 ml of anhydrous DMF, 500 mg (1.81 mmol) of the compound from Ex. 316A were added and the mixture was heated to 80° C. in a microwave (Biotage Initiator) for 1 h. Then 10 ml of water were added to the reaction mixture, which was stirred at RT for 10 min. The product which precipitated out was filtered off with suction, washed with water and then dried under high vacuum. 390 mg (75% of theory, 85% purity) of the title compound were obtained.

¹H-NMR (400 MHz, DMSO-d₆, δ/ppm): 10.08 (s, 1H), 4.09 (m, 2H), 2.77 (s, 3H), 2.65 (m, 4H), 2.26 (m, 1H), 1.15 (d, 3H), 1.06-0.96 (m, 1H), 0.88-0.81 (m, 2H).

LC/MS (Method 1, ESIpos): Rₜ=1.92 min, m/z=369.1 [M+H]⁺.

### Example 348A

### 1-[(3,3-Difluorocyclopentyl)methyl]-5-methyl-3-[(1S,2S)-2-methylcyclopropyl]-2,4-dioxo-1,2,3,4-tetrahydrothieno[2,3-d]pyrimidine-6-carbaldehyde (diastereomer mixture)

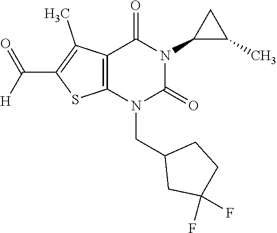 [SMILES: Cc1c(C=O)sc2c1c(=O)n([C@H]1C[C@@H]1C)c(=O)n2CC1CCC(F)(F)C1]

300 mg (1.14 mmol) of the compound from Ex. 303A and 925 mg (2.84 mmol) of caesium carbonate were initially charged in 3 ml of anhydrous DMF, 659 mg (2.27 mmol) of the compound from Ex. 318A were added and the mixture was heated to 80° C. in a microwave (Biotage Initiator) for 18 h. Subsequently, another 165 mg (0.57 mmol) of the compound from Ex. 318A were added and the mixture was heated to 80° C. for a further 6 h. The reaction mixture was then taken up in ethyl acetate and washed with water and saturated aqueous sodium chloride solution. The organic phase was dried over magnesium sulfate, filtered and concentrated on a rotary evaporator. The residue that remained was chromatographed using a silica gel cartridge (Biotage, 100 g SNAP Ultra, cyclohexane/ethyl acetate 2:1). 92 mg (21% of theory) of the title compound were obtained.

¹H-NMR (400 MHz, DMSO-d₆, δ/ppm): 10.08 (s, 1H), 4.03-3.89 (m, 2H), 2.77 (s, 3H), 2.67-2.55 (m, 1H), 2.37-1.86 (m, 6H), 1.59 (m, 1H), 1.16 (d, 3H), 1.06-0.96 (m, 1H), 0.88-0.81 (m, 2H).

LC/MS (Method 1, ESIpos): Rₜ=1.99 min, m/z=383.1 [M+H]⁺.

### Example 349A

### 1-(2-Methoxyethyl)-5-methyl-3-(2-methylcyclopropyl)-2,4-dioxo-1,2,3,4-tetrahydrothieno[2,3-d]pyrimidine-6-carbaldehyde (cis racemat)

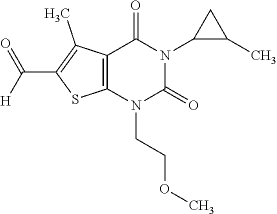 [SMILES: COCCn1c(=O)n(C2CC2C)c(=O)c2c(C)c(C=O)sc21]

Analogously to the method described in Ex. 63A, 685 mg (2.59 mmol) of the compound from Ex. 297A and 1.08 g (7.77 mmol) of 2-bromoethyl methyl ether were used to prepare 538 mg (64% of theory) of the title compound. The reaction time here was 2.5 days.

¹H-NMR (400 MHz, DMSO-d₆, δ/ppm): 10.07 (s, 1H), 4.21-4.10 (m, 1H), 4.06-3.93 (m, 1H), 3.72-3.56 (m, 2H), 3.23 (s, 3H), 2.76 (s, 3H), 2.66 (td, 1H), 1.29-1.20 (m, 2H), 0.86 (d, 3H), 0.57-0.48 (m, 1H).

LC/MS (Method 1, ESIpos): Rₜ=1.58 min, m/z=323.11 [M+H]⁺.

### Example 350A

### 1-(2-Methoxyethyl)-5-methyl-3-[(1S,2S)-2-methylcyclopropyl)-2,4-dioxo-1,2,3,4-tetrahydrothieno[2,3-d]pyrimidine-6-carbaldehyde

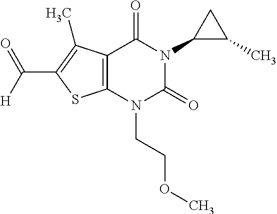 [SMILES: COCCn1c(=O)n([C@H]2C[C@@H]2C)c(=O)c2c(C)c(C=O)sc21]

To a suspension of 32.50 g (123 mmol) of the compound from Ex. 303A in 450 ml of DMF were added 42.49 g (307 mmol) potassium carbonate and the mixture was stirred at RT for 15 min, before 51.27 g (369 mmol) of 2-bromoethyl methyl ether were added. The reaction mixture was stirred at RT for 4 days. Then 2.25 litres of water were added, and the mixture was extracted with 1 litre of ethyl acetate. After phase separation, the solids present in the water phase were filtered off with suction, washed with a little ethyl acetate and dried under reduced pressure (10.70 g of product). The filtrate was extracted with 1 litre of ethyl acetate. The combined ethyl acetate phases were washed with 500 ml of 10% sodium chloride solution, dried over anhydrous magnesium sulfate, filtered and concentrated. The remaining residue was stirred with 145 ml of ethyl acetate at RT. The solids were filtered off with suction, washed with a little ethyl acetate and dried under reduced pressure (19.70 g of product). The mother liquor was concentrated and the residue was chromatographed using silica gel (500 g of 0.04-0.063 mm silica gel, petroleum ether/ethyl acetate 7:3). The product fractions were combined and concentrated, and the residue was dried under reduced pressure (3.16 g of product). In this way, a total of 33.56 g (84% of theory) of the title compound was obtained.

¹H-NMR (500 MHz, DMSO-d₆, δ/ppm): 10.07 (s, 1H), 4.14-3.98 (m, 2H), 3.63 (t, 2H), 3.24 (s, 3H), 2.76 (s, 3H), 2.30-2.23 (m, 1H), 1.15 (d, 3H), 1.08-0.96 (m, 1H), 0.90-0.80 (m, 2H).

LC/MS (Method 6, ESIpos): Rₜ=1.16 min, m/z=323 [M+H]⁺.

### Example 351A

### 5-Methyl-3-(2-methylcyclopropyl)-2,4-dioxo-1-[2-(trifluoromethoxy)ethyl]-1,2,3,4-tetrahydrothieno[2,3-d]pyrimidine-6-carbaldehyde (trans racemate)

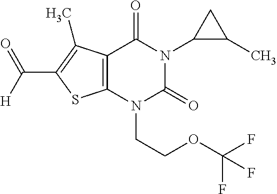 [SMILES: Cc1c(C=O)sc2c1c(=O)n(C1CC1C)c(=O)n2CCOC(F)(F)F]

1.21 g (4.58 mmol) of the compound from Ex. 34A and 1.33 g (9.61 mmol) of potassium carbonate were stirred in 20 ml of anhydrous DMF at RT for 15 min, before 972 mg (5.04 mmol) of 1-bromo-2-(trifluoromethoxy)ethane[commercially available; lit.: P. E. Aldrich, W. A. Sheppard, J. Org. Chem. 29 (1), 11-15 (1964)] were added. Then the reaction mixture was stirred first at RT for 2.5 days and then at 50° C. for 6 h. After being left to stand to RT for a further 2.5 days, the mixture was admixed with water and extracted with ethyl acetate. The organic extract was washed successively with water and saturated sodium chloride solution, dried over anhydrous magnesium sulfate and concentrated. The residue was purified by means of MPLC (Biotage Isolera One, SNAP Ultra cartridge, 100 g of silica gel, cyclohexane/ethyl acetate gradient). After concentration of the product fractions and drying under high vacuum, 1.17 g (67% of theory) of the title compound were obtained.

¹H-NMR (400 MHz, DMSO-d₆, δ/ppm): 10.08 (s, 1H), 4.45-4.35 (m, 2H), 4.32-4.17 (m, 2H), 2.77 (s, 3H), 2.31-2.24 (m, 1H), 1.16 (d, 3H), 1.09-0.95 (m, 1H), 0.90-0.80 (m, 2H).

LC/MS (Method 1, ESIpos): Rₜ=1.93 min, m/z=377.08 [M+H]⁺.

### Example 352A

### 5-Methyl-3-(2-methylcyclopropyl)-2,4-dioxo-1-{2-[(trifluoromethyl)sulfanyl]ethyl}-1,2,3,4-tetrahydrothieno[2,3-d]pyrimidine-6-carbaldehyde (trans racemate)

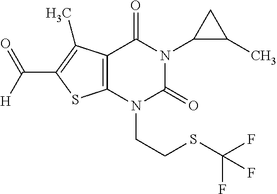 [SMILES: Cc1c(C=O)sc2c1c(=O)n(C1CC1C)c(=O)n2CCSC(F)(F)F]

200 mg (0.76 mmol) of the compound from Ex. 34A and 261 mg (1.89 mmol) of potassium carbonate were initially charged in 2.9 ml of anhydrous DMF, 456 mg (2.12 mmol) of 1-bromo-2-[(trifluoromethyl)sulfanyl]ethane were added and the mixture was heated to 80° C. in a microwave (Biotage Initiator) for 1 h. Then water was then added to the reaction mixture, which was stirred at RT for 10 min. The product which precipitated out was filtered off with suction, washed with water and then dried under high vacuum. 267 mg (82% of theory, 91% purity) of the title compound were obtained.

¹H-NMR (400 MHz, DMSO-d₆, δ/ppm): 10.08 (s, 1H), 4.17 (m, 2H), 3.34 (t, 2H), 2.77 (s, 3H), 2.26 (m, 1H), 1.15 (d, 3H), 1.05-0.95 (m, 1H), 0.89-0.83 (m, 2H).

LC/MS (Method 2, ESIpos): Rₜ=1.06 min, m/z=393.0 [M+H]⁺.

### Example 353A

### 5-Methyl-3-[(1S,2S)-2-methylcyclopropyl]-1-(oxetan-2-ylmethyl)-2,4-dioxo-1,2,3,4-tetrahydrothieno[2,3-d]pyrimidine-6-carbaldehyde (diastereomer mixture)

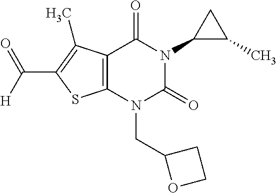 [SMILES: Cc1c(C=O)sc2c1c(=O)n([C@H]1C[C@@H]1C)c(=O)n2CC1CCO1]

400 mg (1.52 mmol) of the compound from Ex. 303A and 523 mg (1.89 mmol) of potassium carbonate were initially charged in 5.7 ml of anhydrous DMF, 686 mg (4.54 mmol) of 2-(bromomethyl)oxetane were added and the mixture was heated in a microwave (Biotage Initiator) first to 80° C. for 1 h and then to 100° C. for 2 h. Then 100 ml of water were added to the reaction mixture, which was stirred at RT for 10 min. The mixture was extracted three times with 50 ml each time of ethyl acetate, and the combined organic phases were washed twice with saturated aqueous sodium chloride solution, dried over sodium sulfate, filtered and concentrated on a rotary evaporator. The residue was washed with water, then taken up in a methanol/dichloromethane mixture, concentrated again on a rotary evaporator and finally dried under high vacuum. 470 mg (85% of theory, 91% purity) of the title compound were obtained.

¹H-NMR (400 MHz, DMSO-d₆, δ/ppm): 10.07 (s, 1H), 5.00 (m, 1H), 4.46 (m, 2H), 4.18 (m, 2H), 2.76 (s, 3H), 2.70 (m, 2H), 2.27 (m, 1H), 1.15 (d, 3H), 1.07-0.99 (m, 1H), 0.88-0.83 (m, 2H).

LC/MS (Method 1, ESIpos): Rₜ=1.55 min, m/z=335.1 [M+H]⁺.

### Example 354A

### 5-Methyl-3-[(1S,2S)-2-methylcyclopropyl]-1-(oxetan-3-ylmethyl)-2,4-dioxo-1,2,3,4-tetrahydrothieno[2,3-d]pyrimidine-6-carbaldehyde

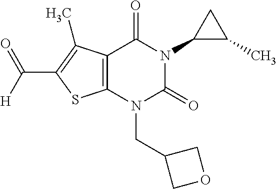 [SMILES: Cc1c(C=O)sc2c1c(=O)n([C@H]1C[C@@H]1C)c(=O)n2CC1COC1]

100 mg (0.38 mmol) of the compound from Ex. 303A and 131 mg (0.47 mmol) of potassium carbonate were initially charged in 1.4 ml of anhydrous DMF, 122 mg (1.15 mmol) of 3-(chloromethyl)oxetane were added and the mixture was heated in a microwave (Biotage Initiator) first to 80° C. for 1 h and then to 100° C. for 5 h. Then 10 ml of water were added to the reaction mixture, which was stirred at RT for 10 min. The precipitate present was filtered off, washed with water, then taken up in a methanol/dichloromethane mixture, concentrated again on a rotary evaporator and dried under high vacuum. The aqueous filtrate was extracted twice with 10 ml each time of ethyl acetate, and the combined organic phases were washed with saturated aqueous sodium chloride solution, dried over sodium sulfate, filtered and concentrated on a rotary evaporator. The product thus obtained was combined with the product obtained from the filtration. A total of 102 mg (81% of theory) of the title compound were obtained.

### Example 355A

### 5-Methyl-3-(2-methylcyclopropyl)-2,4-dioxo-1-[(2R)-tetrahydrofuran-2-ylmethyl]-1,2,3,4-tetrahydrothieno[2,3-d]pyrimidine-6-carbaldehyde (trans diastereomer mixture)

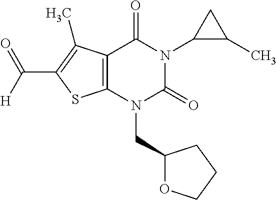 [SMILES: Cc1c(C=O)sc2c1c(=O)n(C1CC1C)c(=O)n2C[C@H]1CCCO1]

200 mg (0.76 mmol) of the compound from Ex. 34A and 261 mg (1.89 mmol) of potassium carbonate were initially charged in 2.9 ml of anhydrous DMF, 394 mg (2.28 mmol) of (2R)-2-(bromomethyl)tetrahydrofuran were added and the mixture was heated to 100° C. in a microwave (Biotage Initiator) for 5.5 h. Water was then added and the reaction mixture was extracted with ethyl acetate. The organic phase was washed with water and with saturated aqueous sodium chloride solution, dried over sodium sulfate, filtered and concentrated on a rotary evaporator. 210 mg (64% of theory, 80% purity) of the title compound were obtained.

LC/MS (Method 6, ESIpos): Rₜ=1.22 min, m/z=349.2 [M+H]⁺.

### Example 356A

### 5-Methyl-3-(2-methylcyclopropyl)-2,4-dioxo-1-(tetrahydrofuran-3-ylmethyl)-1,2,3,4-tetrahydrothieno[2,3-d]pyrimidine-6-carbaldehyde (mixture of all trans stereoisomers)

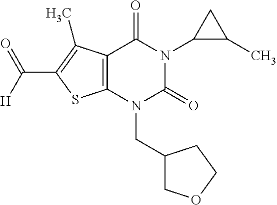 [SMILES: Cc1c(C=O)sc2c1c(=O)n(C1CC1C)c(=O)n2CC1CCOC1]

200 mg (0.76 mmol) of the compound from Ex. 34A and 261 mg (1.89 mmol) of potassium carbonate were initially charged in 3 ml of anhydrous DMF, 374 mg (2.27 mmol) of racemic 3-(bromomethyl)tetrahydrofuran were added and the mixture was heated to 100° C. in a microwave (Biotage Initiator) for 2 h. About 15 ml of water were then added to the reaction mixture, which was extracted three times with 5 ml each time of ethyl acetate. The organic phase was dried over sodium sulfate, filtered and concentrated on a rotary evaporator. 279 mg (96% of theory, 91% purity) of the title compound were obtained.

¹H-NMR (400 MHz, DMSO-d₆, δ/ppm): 10.08 (s, 1H), 3.91 (m, 1H), 3.80 (m, 1H), 3.64 (m, 1H), 3.50 (m, 1H), 2.77 (s, 3H), 2.72 (m, 1H), 2.26 (m, 1H), 1.97 (m, 1H), 1.67 (m, 1H), 1.16 (d, 3H), 1.07-0.97 (m, 1H), 0.89-0.81 (m, 2H).

LC/MS (Method 1, ESIpos): Rₜ=1.59 min, m/z=349.1 [M+H]⁺.

### Example 357A

### 1-(2-Methoxyethyl)-5-methyl-2,4-dioxo-3-[2-(trifluoromethyl)cyclopropyl]-1,2,3,4-tetrahydrothieno[2,3-d]pyrimidine-6-carbaldehyde (trans racemate)

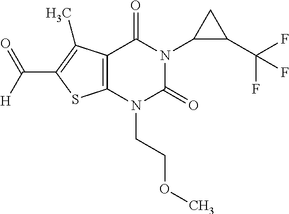 [SMILES: COCCn1c(=O)n(C2CC2C(F)(F)F)c(=O)c2c(C)c(C=O)sc21]

1.03 g (3.22 mmol) of the compound from Ex. 306A and 1.11 g (8.05 mmol) of potassium carbonate were stirred in 15 ml of anhydrous DMF at RT for 15 min, before 1.34 g (9.66 mmol) of 2-bromoethyl methyl ether were added. Then the reaction mixture was stirred first at RT for 2.5 days and then at 55° C. for 4 h. After cooling to RT, the mixture was diluted with 30 ml of ethyl acetate and washed successively with water and saturated sodium chloride solution, dried over anhydrous magnesium sulfate and concentrated. The residue was purified by means of MPLC (Biotage Isolera One, SNAP Ultra cartridge, 50 g of silica gel, cyclohexane/ethyl acetate gradient). After concentration of the product fractions, the solids obtained were stirred in a mixture of 100 ml of cyclohexane and 10 ml of dichloromethane at RT. The purified solids were filtered off with suction and dried under high vacuum. 608 mg (50% of theory) of the title compound were obtained.

¹H-NMR (400 MHz, DMSO-d₆, δ/ppm): 10.08 (s, 1H), 4.15-3.99 (m, 2H), 3.64 (t, 2H), 3.25 (s, 3H), 2.99 (ddd, 1H), 2.77 (s, 3H), 2.27 (dtd, 1H), 1.52 (q, 1H), 1.40-1.31 (m, 1H).

LC/MS (Method 2, ESIpos): Rₜ=0.90 min, m/z=377 [M+H]⁺.

### Example 358A

### 3-(2-Ethylcyclopropyl)-1-(2-methoxyethyl)-5-methyl-2,4-dioxo-1,2,3,4-tetrahydrothieno[2,3-d]pyrimidine-6-carbaldehyde (trans racemate)

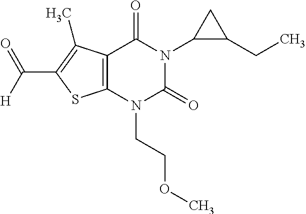 [SMILES: CCC1CC1n1c(=O)c2c(C)c(C=O)sc2n(CCOC)c1=O]

820 mg (2.95 mmol) of the compound from Ex. 309A and 1.02 g (7.37 mmol) of potassium carbonate were stirred in 12 ml of anhydrous DMF at RT for 15 min, before 1.23 g (8.84 mmol) of 2-bromoethyl methyl ether were added. Then the reaction mixture was stirred at 100° C. in a microwave oven (Biotage Initiator with dynamic control of irradiation power) for 4 h. After cooling to RT, the mixture was diluted with 30 ml of ethyl acetate and washed successively with water and saturated sodium chloride solution, dried over anhydrous magnesium sulfate and concentrated. The residue was purified by means of MPLC (Biotage Isolera One, SNAP Ultra cartridge, 50 g of silica gel, cyclohexane/ethyl acetate gradient). After concentration of the product fractions, the solids obtained were dried under high vacuum. 867 mg (87% of theory) of the title compound were obtained.

¹H-NMR (400 MHz, DMSO-d₆, δ/ppm): 10.07 (s, 1H), 4.12-3.99 (m, 2H), 3.63 (t, 2H), 3.24 (s, 3H), 2.76 (s, 3H), 2.38-2.29 (m, 1H), 1.61 (dquin, 1H), 1.25 (dquin, 1H), 1.11-0.95 (m, 1H), 1.00 (t, 3H), 0.90-0.77 (m, 2H).

LC/MS (Method 1, ESIpos): Rₜ=1.82 min, m/z=337.12 [M+H]⁺.

### Example 359A

### 3-(2-Methoxycyclopropyl)-1-(2-methoxyethyl)-5-methyl-2,4-dioxo-1,2,3,4-tetrahydrothieno[2,3-d]pyrimidine-6-carbaldehyde (trans racemate)

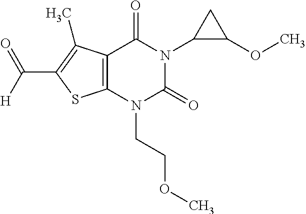 [SMILES: COCCn1c(=O)n(C2CC2OC)c(=O)c2c(C)c(C=O)sc21]

817 mg (2.92 mmol) of the compound from Ex. 312A and 1.01 g (7.29 mmol) of potassium carbonate were stirred in 14 ml of anhydrous DMF at RT for 15 min, before 1.22 g (8.74 mmol) of 2-bromoethyl methyl ether were added. Subsequently, the reaction mixture was stirred at RT for 3 days. Thereafter, 400 ml of water were added. In the course of this, a portion of the product precipitated out, which was isolated by suction filtration. The filtrate was extracted with ethyl acetate. The organic extract was washed successively with water and saturated aqueous sodium chloride solution. After drying over anhydrous magnesium sulfate, the mixture was filtered and the concentrated. The residue that remained was combined with the product previously isolated and purified by means of MPLC (Biotage Isolera One, SNAP Ultra cartridge, 50 g of silica gel, cyclohexane/ethyl acetate gradient). After the product fractions had been concentrated, the product was dried under high vacuum. 676 mg (66% of theory, 96% purity) of the title compound were obtained.

¹H-NMR (400 MHz, DMSO-d₆, δ/ppm): 10.07 (s, 1H), 4.18-3.98 (m, 2H), 3.70-3.57 (m, 2H), 3.49-3.41 (m, 1H), 3.24 (s, 3H), 3.23 (s, 3H), 2.76 (s, 3H), 2.64 (dt, 1H), 1.33 (td, 1H), 0.91 (ddd, 1H).

LC/MS (Method 1, ESIpos): Rₜ=1.29 min, m/z=339.10 [M+H]⁺.

### Example 360A tert-Butyl {2-[({1-(2-methoxyethyl)-5-methyl-3-[(1S,2S)-2-methylcyclopropyl]-2,4-dioxo-1,2,3,4-tetrahydrothieno[2,3-d]pyrimidin-6-yl}methyl)amino]ethyl}carbamate

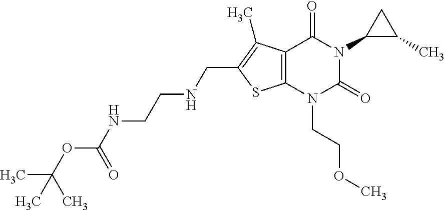 [SMILES: COCCn1c(=O)n([C@H]2C[C@@H]2C)c(=O)c2c(C)c(CNCCNC(=O)OC(C)(C)C)sc21]

To a solution of 208.7 g (647 mmol) of the compound from Ex. 350A in 5 litres of ethanol were added, at RT, 155.6 g (971 mmol) of tert-butyl (2-aminoethyl)carbamate and 55.6 ml (971 mmol) of acetic acid, and then the mixture was stirred at 55° C. for 15 h. After cooling to RT, a total of 44.1 g (1.17 mol) of solid sodium borohydride were added in three portions to the reaction mixture within 90 min. In the course of this, vigorous evolution of gas occurred. One hour after the last addition of sodium borohydride, 417 ml of water were added to the reaction mixture, which was stirred vigorously at RT for 10 min. The mixture was then concentrated to dryness on a rotary evaporator. The residue that remained was taken up in a mixture of 2.14 litres of toluene and 856 ml of 1 M sodium hydroxide solution, and stirred at RT. After phase separation, the aqueous phase was extracted with 1 litre of toluene. The combined toluene phases were then washed twice with 800 ml each time of water, dried over anhydrous magnesium sulfate and concentrated. The residue was chromatographed using silica gel (10 kg of 0.063-0.2 mm silica gel, dichloromethane/methanol 97:3). After concentration of the product fractions and drying under reduced pressure, 291 g (96% of theory) of the title compound were obtained.

¹H-NMR (400 MHz, DMSO-d₆, δ/ppm): 6.84-6.61 (m, 1H), 4.06-3.89 (m, 2H), 3.78-3.74 (m, 2H), 3.61 (t, 2H), 3.24 (s, 3H), 3.01 (q, 2H), 2.59-2.53 (m, 2H), 2.42-2.33 (m, 1H), 2.26-2.19 (m, 1H), 1.37 (s, 9H), 1.15 (d, 3H), 1.06-0.91 (m, 1H), 0.87-0.77 (m, 2H).

### Example 361A

### 6-{[(2-Aminoethyl)amino]methyl}-3-cyclopropyl-1-[(3,3-difluorocyclobutyl)methyl]-5-methylthieno[2,3-d]pyrimidine-2,4(1H,3H)-dione

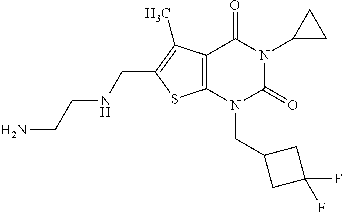 [SMILES: Cc1c(CNCCN)sc2c1c(=O)n(C1CC1)c(=O)n2CC1CC(F)(F)C1]

150 mg (0.423 mmol) of the compound from Ex. 328A were dissolved in a mixture of 4 ml of methanol and 2 ml of dichloromethane. Subsequently, 170 μl (2.54 mmol) of 1,2-diaminoethane and 97 μl (1.69 mmol) of acetic acid were added at RT. After 30 min, a further 106 mg (1.69 mmol) of sodium cyanoborohydride were added. Subsequently, the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The crude product thus obtained, after drying under high vacuum, gave 189 mg (95% of theory, 85% purity) of the title compound, which was used for subsequent reactions without further purification.

LC/MS (Method 1, ESIpos): Rₜ=0.73 min, m/z=399.17 [M+H]⁺.

### Example 362A

### 1,5-Dimethyl-3-[(1S,2S)-2-methylcyclopropyl]-2,4-dioxo-1,2,3,4-tetrahydrothieno[2,3-d]pyrimidine-6-carbaldehyde

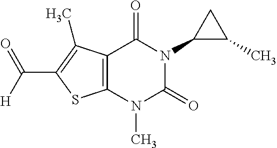 [SMILES: Cc1c(C=O)sc2c1c(=O)n([C@H]1C[C@@H]1C)c(=O)n2C]

300 mg (1.14 mmol) of the compound from Ex. 303A were dissolved in 5 ml of anhydrous DMF, 392 mg (2.84 mmol) of potassium carbonate were added, and the mixture was stirred at RT for 15 min. Then 212 μl (3.41 mmol) of methyl iodide were added. After the reaction mixture had been stirred at 50° C. for 1 h, it was poured onto water and acidified with 1 M hydrochloric acid. In the course of this, the product precipitated out. After stirring at RT for 1 h, the solids were filtered off with suction, washed with a little water and dried under high vacuum. 313 mg (99% of theory) of the title compound were obtained.

¹H-NMR (500 MHz, DMSO-d₆, δ/ppm): 10.07 (s, 1H), 3.43 (s, 3H), 2.77 (s, 3H), 2.29-2.22 (m, 1H), 1.15 (d, 3H), 1.06-0.95 (m, 1H), 0.90-0.81 (m, 2H).

LC/MS (Method 1, ESIpos): Rₜ=1.54 min, m/z=279.08 [M+H]⁺.

### Example 363A

### 6-{[(2-Aminoethyl)amino]methyl}-1,5-dimethyl-3-(2-methylcyclopropyl)thieno[2,3-d]pyrimidine-2,4(1H,3H)-dione (trans racemate)

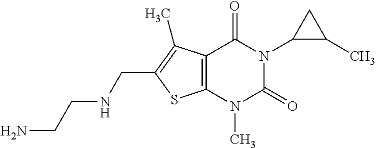 [SMILES: Cc1c(CNCCN)sc2c1c(=O)n(C1CC1C)c(=O)n2C]

To a solution of 160 mg (0.58 mmol) of the compound from Ex. 336A in 4 ml of methanol and 1 ml of dichloromethane were added 130 μl (2.3 mmol) of acetic acid and 230 μl (3.45 mmol) of 1,2-ethylenediamine. The mixture was then stirred at RT for 30 min. The reaction mixture was then diluted with 4 ml of methanol, and 145 mg (2.3 mmol) of sodium cyanoborohydride were added. The mixture was stirred at 60° C. for 8 h and then at RT for a further 60 h. Then 2 M sodium hydroxide solution and a little sodium chloride were added to the mixture, which was extracted three times with ethyl acetate. The combined organic phases were washed with saturated aqueous sodium chloride solution, dried over sodium sulfate, filtered and concentrated on a rotary evaporator. The residue was taken up in 5 ml of ethyl acetate, washed with a mixture of 1 ml each of saturated aqueous sodium chloride solution and 1M sodium hydroxide solution, dried over sodium sulfate, filtered and concentrated again on a rotary evaporator. After the residue had been dried under high vacuum, 173 mg (93% of theory) of the title compound were obtained, which was used further without further purification.

### Example 364A

### 6-{[(2-Aminoethyl)amino]methyl}-1-ethyl-5-methyl-3-(2-methylcyclopropyl)thieno[2,3-d]pyrimidine-2,4(1H,3H)-dione (trans racemate)

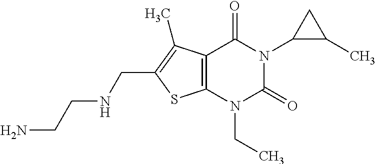 [SMILES: CCn1c(=O)n(C2CC2C)c(=O)c2c(C)c(CNCCN)sc21]

To a solution of 220 mg (94% purity, 0.71 mmol) of the compound from Ex. 337A in 3.8 ml of methanol and 0.9 ml of dichloromethane were added 160 μl (2.83 mmol) of acetic acid and 280 μl (4.25 mmol) of 1,2-ethylenediamine. The mixture was then stirred at RT for 30 min. Then 178 mg (2.83 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. The residue was dried under high vacuum. 317 mg (>100% of theory) of the crude title compound were obtained, which was used further without further purification.

### Example 365A

### 6-{[(2-Aminoethyl)amino]methyl}-5-methyl-3-(2-methylcyclopropyl)-1-propylthieno[2,3-d]pyrimidine-2,4(1H,3H)-dione (trans racemate)

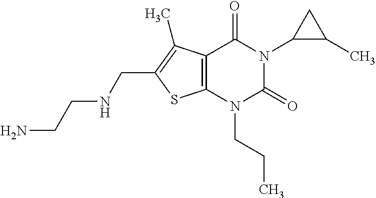 [SMILES: CCCn1c(=O)n(C2CC2C)c(=O)c2c(C)c(CNCCN)sc21]

To a solution of 205 mg (0.67 mmol) of the compound from Ex. 338A in 4.6 ml of methanol and 1.2 ml of dichloromethane were added 150 μl (2.68 mmol) of acetic acid and 270 μl (4.02 mmol) of 1,2-ethylenediamine. The mixture was then stirred at RT for 30 min. Then 168 mg (2.66 mmol) of sodium cyanoborohydride were added, and stirring of the reaction mixture was continued at 60° C. overnight. Then 6 ml of 1 M sodium hydroxide solution and 6 ml of saturated aqueous sodium chloride solution were added to the mixture, which was extracted three times with 10 ml each time of ethyl acetate. The combined organic phases were washed with saturated aqueous sodium chloride solution, dried over sodium sulfate, filtered and concentrated on a rotary evaporator. After the residue had been dried under high vacuum, 266 mg (>100% of theory) of the crude title compound were obtained, which was used further without further purification.

### Example 366A

### 6-{[(2-Aminoethyl)amino]methyl}-1-butyl-5-methyl-3-[(1S,2S)-2-methylcyclopropyl]thieno[2,3-d]pyrimidine-2,4(1H,3H)-dione

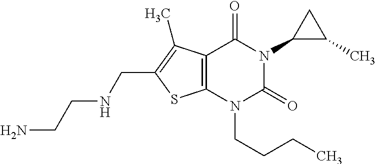 [SMILES: CCCCn1c(=O)n([C@H]2C[C@@H]2C)c(=O)c2c(C)c(CNCCN)sc21]

To a solution of 96 mg (0.26 mmol, 87% purity) of the compound from Ex. 339A in 1.8 ml of methanol and 0.5 ml of dichloromethane were added 60 μl (1.05 mmol) of acetic acid and 110 μl (1.57 mmol) of 1,2-ethylenediamine. The mixture was then stirred at RT for 30 min. Then 66 mg (1.05 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 150 mg (>100% of theory) of the crude title compound were obtained, which was used further without further purification.

### Example 367A

### 6-{[(2-Aminoethyl)amino]methyl}-1-(2-fluoroethyl)-5-methyl-3-(2-methylcyclopropyl)thieno[2,3-d]pyrimidine-2,4(1H,3H)-dione (trans racemate)

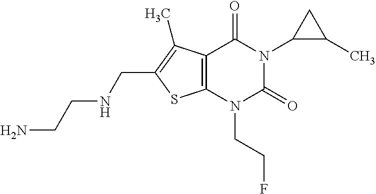 [SMILES: Cc1c(CNCCN)sc2c1c(=O)n(C1CC1C)c(=O)n2CCF]

To a solution of 210 mg (0.68 mmol) of the compound from Ex. 340A in 4 ml of methanol and 1 ml of dichloromethane were added 160 μl (2.72 mmol) of acetic acid and 270 μl (4.01 mmol) of 1,2-ethylenediamine. The mixture was then stirred at RT for 30 min. Then 170 mg (2.72 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 300 mg (>100% of theory) of the crude title compound were obtained, which was used further without further purification.

### Example 368A

### 6-{[(2-Aminoethyl)amino]methyl}-1-(2,2-difluoroethyl)-5-methyl-3-(2-methylcyclopropyl)thieno[2,3-d]pyrimidine-2,4(1H,3H)-dione (trans racemate)

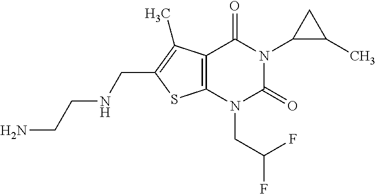 [SMILES: Cc1c(CNCCN)sc2c1c(=O)n(C1CC1C)c(=O)n2CC(F)F]

To a solution of 167 mg (0.51 mmol) of the compound from Ex. 341A in 3.9 ml of methanol and 1 ml of dichloromethane were added 120 μl (2.03 mmol) of acetic acid and 200 μl (3.05 mmol) of 1,2-ethylenediamine. The mixture was then stirred at RT for 30 min. Then 128 mg (2.03 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 279 mg (>100% of theory) of the crude title compound were obtained, which was used further without further purification.

### Example 369A

### 6-{[(2-Aminoethyl)amino]methyl}-5-methyl-3-(2-methylcyclopropyl)-1-(2,2,2-trifluoroethyl)thieno[2,3-d]pyrimidine-2,4(1H,3H)-dione (trans racemate)

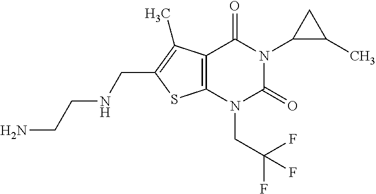 [SMILES: Cc1c(CNCCN)sc2c1c(=O)n(C1CC1C)c(=O)n2CC(F)(F)F]

To a solution of 100 mg (0.28 mmol) of the compound from Ex. 342A in 1.6 ml of methanol and 0.4 ml of dichloromethane were added 60 μl (1.12 mmol) of acetic acid and 110 μl (1.68 mmol) of 1,2-ethylenediamine. The mixture was then stirred at RT for 30 min. Then 70 mg (1.12 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 158 mg (>100% of theory) of the crude title compound were obtained, which was used further without further purification.

### Example 370A

### 6-{[(2-Aminoethyl)amino]methyl}-5-methyl-3-(2-methylcyclopropyl)-1-(4,4,4-trifluorobutyl)thieno[2,3-d]pyrimidine-2,4(1H,3H)-dione (trans racemate)

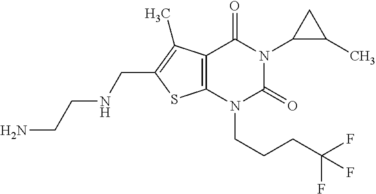 [SMILES: Cc1c(CNCCN)sc2c1c(=O)n(C1CC1C)c(=O)n2CCCC(F)(F)F]

To a solution of 250 mg (0.67 mmol) of the compound from Ex. 343A in 3.7 ml of methanol and 0.9 ml of dichloromethane were added 140 μl (2.46 mmol) of acetic acid and 250 μl (3.70 mmol) of 1,2-ethylenediamine. The mixture was then stirred at RT for 30 min. Then 154 mg (2.45 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 457 mg (>100% of theory) of the crude title compound were obtained, which was used further without further purification.

### Example 371A

### 6-{[(2-Aminoethyl)amino]methyl}-5-methyl-3-(2-methylcyclopropyl)-1-(3,3,4,4-tetrafluorobutyl)thieno[2,3-d]pyrimidine-2,4(1H,3H)-dione (trans racemate)

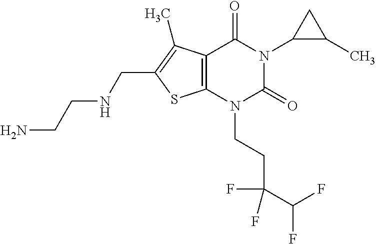 [SMILES: Cc1c(CNCCN)sc2c1c(=O)n(C1CC1C)c(=O)n2CCC(F)(F)C(F)F]

To a solution of 190 mg (0.48 mmol) of the compound from Ex. 344A in 4 ml of methanol and 1 ml of dichloromethane were added 110 μl (1.93 mmol) of acetic acid and 190 μl (2.91 mmol) of 1,2-ethylenediamine. The mixture was then stirred at RT for 30 min. Then 122 mg (1.94 mmol) of sodium cyanoborohydride were added, and stirring of the reaction mixture was continued at 60° C. overnight. Then 5 ml of 1 M sodium hydroxide solution and 5 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 245 mg (>100% of theory) of the crude title compound were obtained, which was used further without further purification.

LC/MS (Method 1, ESIpos): Rₜ=0.84 min, m/z=437.1 [M+H]⁺.

### Example 372A

### 6-{[(2-Aminoethyl)amino]methyl}-1-(cyclobutylmethyl)-5-methyl-3-[(1S,2S)-2-methylcyclopropyl]thieno[2,3-d]pyrimidine-2,4(1H,3H)-dione

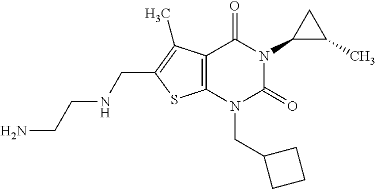 [SMILES: Cc1c(CNCCN)sc2c1c(=O)n([C@H]1C[C@@H]1C)c(=O)n2CC1CCC1]

To a solution of 100 mg (0.30 mmol) of the compound from Ex. 345A in 2.1 ml of methanol and 0.5 ml of dichloromethane were added 70 μl (1.20 mmol) of acetic acid and 120 μl (1.80 mmol) of 1,2-ethylenediamine. The mixture was then stirred at RT for 30 min. Then 75 mg (1.20 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 148 mg (>100% of theory) of the crude title compound were obtained, which was used further without further purification.

### Example 373A

### 6-{[(2-Aminoethyl)amino]methyl}-1-[(2,2-difluorocyclopropyl)methyl]-5-methyl-3-[(1S,2S)-2-methylcyclopropyl]thieno[2,3-d]pyrimidine-2,4(1H,3H)-dione (diastereomer mixture)

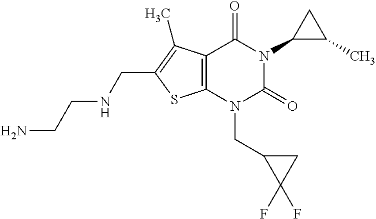 [SMILES: Cc1c(CNCCN)sc2c1c(=O)n([C@H]1C[C@@H]1C)c(=O)n2CC1CC1(F)F]

To a solution of 100 mg (0.28 mmol) of the compound from Ex. 346A in 2 ml of methanol and 0.5 ml of dichloromethane were added 70 μl (1.20 mmol) of acetic acid and 110 μl (1.70 mmol) of 1,2-ethylenediamine. The mixture was then stirred at RT for 30 min. Then 71 mg (1.13 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 153 mg (>100% of theory) of the crude title compound were obtained, which was used further without further purification.

### Example 374A

### 6-{[(2-Aminoethyl)amino]methyl}-1-[(3,3-difluorocyclobutyl)methyl]-5-methyl-3-(2-methylcyclopropyl)thieno[2,3-d]pyrimidine-2,4(1H,3H)-dione (trans racemate)

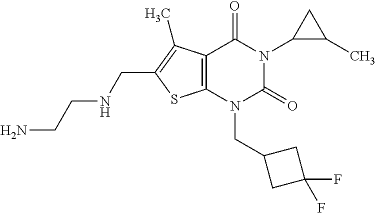 [SMILES: Cc1c(CNCCN)sc2c1c(=O)n(C1CC1C)c(=O)n2CC1CC(F)(F)C1]

To a solution of 200 mg (0.46 mmol, 85% purity) of the compound from Ex. 347A in 2.7 ml of methanol and 0.7 ml of dichloromethane were added 110 μl (1.85 mmol) of acetic acid and 190 μl (2.77 mmol) of 1,2-ethylenediamine. The mixture was then stirred at RT for 30 min. Then 116 mg (1.84 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 210 mg (>100% of theory) of the crude title compound were obtained, which was used further without further purification.

### Example 375A

### 6-{[(2-Aminoethyl)amino]methyl}-1-[(3,3-difluorocyclopentyl)methyl]-5-methyl-3-[(1S,2S)-2-methylcyclopropyl]thieno[2,3-d]pyrimidine-2,4(1H,3H)-dione (diastereomer mixture)

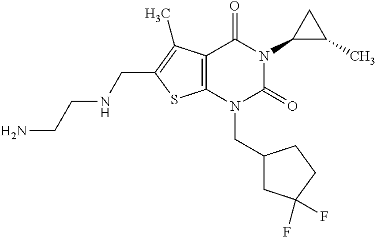 [SMILES: Cc1c(CNCCN)sc2c1c(=O)n([C@H]1C[C@@H]1C)c(=O)n2CC1CCC(F)(F)C1]

Analogously to the method described in Ex. 374A, 90 mg (0.24 mmol) of the compound from Ex. 348A were used to obtain 102 mg (>100% of theory) of the crude title compound, which was used further without further purification.

### Example 376A

### 6-{[(2-Aminoethyl)amino]methyl}-1-(2-methoxyethyl)-5-methyl-3-(2-methylcyclopropyl)thieno[2,3-d]pyrimidine-2,4(1H,3H)-dione (cis racemate)

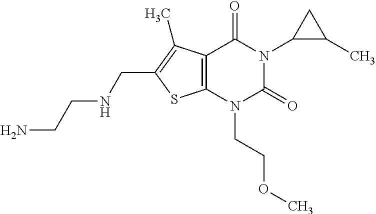 [SMILES: COCCn1c(=O)n(C2CC2C)c(=O)c2c(C)c(CNCCN)sc21]

Analogously to the method described in Ex. 103A, 510 mg (1.58 mmol) of the compound from Ex. 349A, 635 μl (9.49 mmol) of 1,2-diaminoethane and 419 mg (6.33 mmol) of sodium cyanoborohydride were used to prepare 604 mg (91% of theory, 88% purity) of the title compound.

LC/MS (Method 1, ESIpos): Rₜ=0.58 min, m/z=307.11 [M+H−C₂H₈N₂]⁺.

### Example 377A

### 6-{[(2-Aminoethyl)amino]methyl}-1-(2-methoxyethyl)-5-methyl-3-[(1S,2S)-2-methylcyclopropyl]thieno[2,3-d]pyrimidine-2,4(1H,3H)-dione dihydrochloride

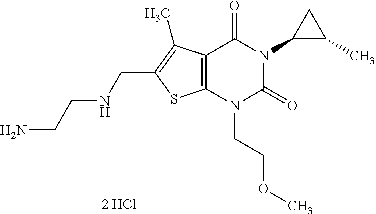 [SMILES: COCCn1c(=O)n([C@H]2C[C@@H]2C)c(=O)c2c(C)c(CNCCN)sc21]

290 g (621 mmol) of the compound from Ex. 360A were dissolved in 1 litre of dioxane, and 2.9 litres of a 4 M solution of hydrogen chloride in dioxane were added at RT. The reaction mixture was stirred at RT for 16 h. Then it was diluted with 2.5 litres of toluene and concentrated again. The residue obtained was taken up in 2.5 litres each time of toluene twice more and concentrated again each time. Then the product was freed of solvent residues under high vacuum. 273 g (99% of theory) of the title compound were obtained.

¹H-NMR (400 MHz, DMSO-d₆, δ/ppm): 9.82 (br. s, 2H), 8.42 (br. s, 3H), 4.37 (br. s, 2H), 4.08-3.91 (m, 2H), 3.64 (t, 2H), 3.29-3.20 (m, 4H), 3.25 (s, 3H), 2.48 (s, 3H), 2.30-2.22 (m, 1H), 1.15 (d, 3H), 1.06-0.93 (m, 1H), 0.90-0.77 (m, 2H).

LC/MS (Method 2, ESIpos): Rₜ=0.40 min, m/z=367 [M+H]⁺.

### Example 378A

### 6-{[(2-Aminoethyl)amino]methyl}-5-methyl-3-(2-methylcyclopropyl)-1-[2-(trifluoromethoxy)ethyl]thieno[2,3-d]pyrimidine-2,4(1H,3H)-dione (trans racemate)

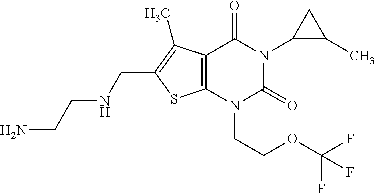 [SMILES: Cc1c(CNCCN)sc2c1c(=O)n(C1CC1C)c(=O)n2CCOC(F)(F)F]

870 mg (2.31 mmol) of the compound from Ex. 351A were dissolved in a mixture of 20 ml of methanol and 7.5 ml of dichloromethane. Subsequently, 927 μl (13.9 mmol) of 1,2-diaminoethane and 529 μl (9.25 mmol) of acetic acid were added at RT. After 30 min, a further 612 mg (9.25 mmol) of sodium cyanoborohydride were added. Subsequently, the mixture was stirred at 60° C. for about 16 h. After cooling to RT, 2 M sodium hydroxide solution was added and extraction was effected with ethyl acetate. The organic extract was washed with saturated sodium chloride solution, dried over anhydrous magnesium sulfate, filtered and concentrated. The residue that remained was purified by means of MPLC (Biotage Isolera One, SNAP Ultra cartridge, 50 g of silica gel, dichloromethane/methanol gradient). After concentration of the product fractions and drying under high vacuum, 550 mg (53% of theory, 94% purity) of the title compound were obtained.

¹H-NMR (400 MHz, DMSO-d₆, δ/ppm): 6.35 (broad, 3H), 4.39 (t, 2H), 4.24-4.07 (m, 2H), 3.81 (s, 2H), 2.93-2.82 (m, 2H), 2.77-2.68 (m, 2H), 2.35 (s, 3H), 2.29-2.22 (m, 1H), 1.15 (d, 3H), 1.04-0.92 (m, 1H), 0.89-0.77 (m, 2H).

LC/MS (Method 1, ESIpos): Rₜ=0.83 min, m/z=421.15 [M+H]⁺.

### Example 379A

### 6-{[(2-Aminoethyl)amino]methyl}-5-methyl-3-(2-methylcyclopropyl)-1-{2-[(trifluoromethyl)sulfanyl]ethyl}thieno[2,3-d]pyrimidine-2,4(1H,3H)-dione (trans racemate)

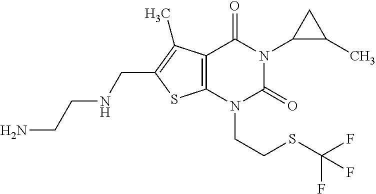 [SMILES: Cc1c(CNCCN)sc2c1c(=O)n(C1CC1C)c(=O)n2CCSC(F)(F)F]

To a solution of 265 mg (0.68 mmol) of the compound from Ex. 352A in 6 ml of methanol and 2 ml of dichloromethane were added 160 μl (2.70 mmol) of acetic acid and 270 μl (4.07 mmol) of 1,2-ethylenediamine. The mixture was then stirred at RT for 30 min. Then 170 mg (2.70 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 360 mg (>100% of theory) of the crude title compound were obtained, which was used further without further purification.

### Example 380A

### 6-{[(2-Aminoethyl)amino]methyl}-5-methyl-3-[(1S,2S)-2-methylcyclopropyl]-1-(oxetan-2-ylmethyl)thieno[2,3-d]pyrimidine-2,4(1H,3H)-dione (diastereomer mixture)

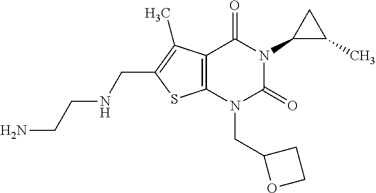 [SMILES: Cc1c(CNCCN)sc2c1c(=O)n([C@H]1C[C@@H]1C)c(=O)n2CC1CCO1]

To a solution of 470 mg (1.41 mmol) of the compound from Ex. 353A in 10 ml of methanol and 3 ml of dichloromethane were added 320 μl (5.63 mmol) of acetic acid and 560 μl (8.45 mmol) of 1,2-ethylenediamine. The mixture was then stirred at RT for 30 min. Then 353 mg (5.62 mmol) of sodium cyanoborohydride were added, and stirring of the reaction mixture was continued at 60° C. overnight. Then 5 ml of 1 M sodium hydroxide solution and 5 ml of saturated aqueous sodium chloride solution were added to the mixture, which was extracted three times with 10 ml each time of ethyl acetate. The combined organic phases were washed with saturated aqueous sodium chloride solution, dried over sodium sulfate, filtered and concentrated on a rotary evaporator. After the residue had been dried under high vacuum, 460 mg (86% of theory) of the crude title compound were obtained, which was used further without further purification.

### Example 381A

### 6-{[(2-Aminoethyl)amino]methyl}-5-methyl-3-[(1S,2S)-2-methylcyclopropyl]-1-(oxetan-3-ylmethyl)thieno[2,3-d]pyrimidine-2,4(1H,3H)-dione

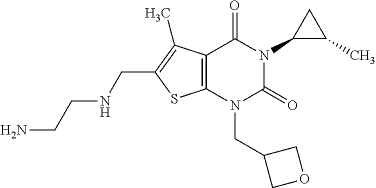 [SMILES: Cc1c(CNCCN)sc2c1c(=O)n([C@H]1C[C@@H]1C)c(=O)n2CC1COC1]

To a solution of 100 mg (0.30 mmol) of the compound from Ex. 354A in 2 ml of methanol and 0.5 ml of dichloromethane were added 70 μl (1.20 mmol) of acetic acid and 120 μl (1.80 mmol) of 1,2-ethylenediamine. The mixture was then stirred at RT for 30 min. Then 75 mg (1.19 mmol) of sodium cyanoborohydride were added, and stirring of the reaction mixture was continued at 60° C. overnight. Then 3 ml of 1 M sodium hydroxide solution and 3 ml of saturated aqueous sodium chloride solution were added to the mixture, which was extracted three times with 5 ml each time of ethyl acetate. The combined organic phases were washed with saturated aqueous sodium chloride solution, dried over sodium sulfate, filtered and concentrated on a rotary evaporator. After the residue had been dried under high vacuum, 150 mg (>100% of theory) of the crude title compound were obtained, which was used further without further purification.

### Example 382A

### 6-{[(2-Aminoethyl)amino]methyl}-5-methyl-3-(2-methylcyclopropyl)-1-[(2R)-tetrahydrofuran-2-ylmethyl]thieno[2,3-d]pyrimidine-2,4(1H,3H)-dione (trans-diastereomer mixture)

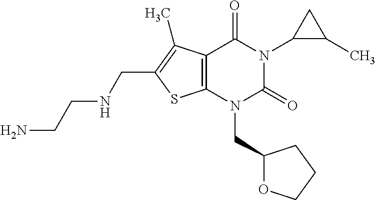 [SMILES: Cc1c(CNCCN)sc2c1c(=O)n(C1CC1C)c(=O)n2C[C@H]1CCCO1]

To a solution of 210 mg (0.48 mmol, 80% purity) of the compound from Ex. 355A in 4.2 ml of methanol and 1 ml of dichloromethane were added 110 μl (1.93 mmol) of acetic acid and 190 μl (2.90 mmol) of 1,2-ethylenediamine. The mixture was then stirred at RT for 30 min. The mixture was then diluted with a further 4 ml of methanol, and 121 mg (1.93 mmol) of sodium cyanoborohydride were added. The reaction mixture was stirred at 60° C. for 8 h and then overnight at RT. Then 6 ml of 1 M sodium hydroxide solution and 6 ml of saturated aqueous sodium chloride solution were added to the mixture, which was extracted three times with 10 ml each time of ethyl acetate. The combined organic phases were washed with saturated aqueous sodium chloride solution, dried over sodium sulfate, filtered and concentrated on a rotary evaporator. After the residue had been dried under high vacuum, 233 mg (>100% of theory) of the crude title compound were obtained, which was used further without further purification.

### Example 383A

### 6-{[(2-Aminoethyl)amino]methyl}-5-methyl-3-(2-methylcyclopropyl)-1-(tetrahydrofuran-3-ylmethyl)thieno[2,3-d]pyrimidine-2,4(1H,3H)-dione (mixture of all trans stereoisomers)

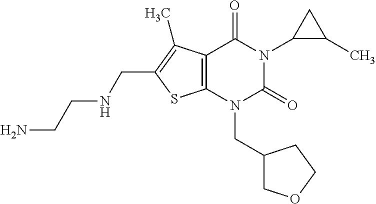 [SMILES: Cc1c(CNCCN)sc2c1c(=O)n(C1CC1C)c(=O)n2CC1CCOC1]

Analogously to the method described in Ex. 374A, 264 mg (0.69 mmol, 91% purity) of the compound from Ex. 356A were used to obtain 372 mg (>100% of theory) of the crude title compound, which was used further without further purification.

### Example 384A

### 6-{[(2-Aminoethyl)amino]methyl}-1-(2-methoxyethyl)-5-methyl-3-[2-(trifluoromethyl)cyclopropyl]thieno[2,3-d]pyrimidine-2,4(1H,3H)-dione (trans racemate)

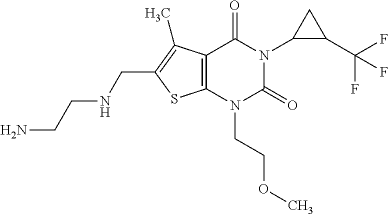 [SMILES: COCCn1c(=O)n(C2CC2C(F)(F)F)c(=O)c2c(C)c(CNCCN)sc21]

Analogously to the method described in Ex. 103A, 300 mg (0.797 mmol) of the compound from Ex. 357A, 320 μl (4.78 mmol) of 1,2-diaminoethane and 211 mg (3.19 mmol) of sodium cyanoborohydride were used to prepare 411 mg (99% of theory, 80% purity) of the title compound.

LC/MS (Method 1, ESIpos): Rₜ=0.74 min, m/z=421.15 [M+H]⁺.

### Example 385A

### 6-{[(2-Aminoethyl)amino]methyl}-3-(2-ethylcyclopropyl)-1-(2-methoxyethyl)-5-methylthieno[2,3-d]pyrimidine-2,4(1H,3H)-dione (trans racemate)

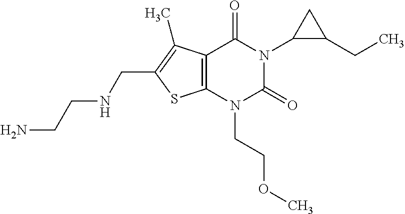 [SMILES: CCC1CC1n1c(=O)c2c(C)c(CNCCN)sc2n(CCOC)c1=O]

Analogously to the method described in Ex. 103A, 275 mg (0.817 mmol) of the compound from Ex. 358A, 328 μl (4.91 mmol) of 1,2-diaminoethane and 216 mg (3.27 mmol) of sodium cyanoborohydride were used to prepare 323 mg (86% of theory, 83% purity) of the title compound.

LC/MS (Method 1, ESIpos): Rₜ=0.73 min, m/z=321.13 [M+H−C₂H₈N₂]⁺.

### Example 386A

### 6-{[(2-Aminoethyl)amino]methyl}-3-(2-methoxycyclopropyl)-1-(2-methoxyethyl)-5-methylthieno[2,3-d]pyrimidine-2,4(1H,3H)-dione (trans racemate)

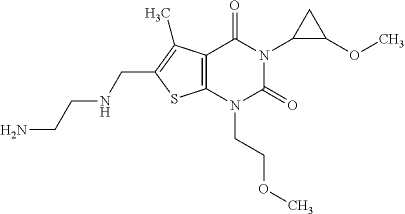 [SMILES: COCCn1c(=O)n(C2CC2OC)c(=O)c2c(C)c(CNCCN)sc21]

Analogously to the method described in Ex. 103A, 320 mg (0.946 mmol) of the compound from Ex. 359A, 379 μl (5.67 mmol) of 1,2-diaminoethane and 250 mg (3.78 mmol) of sodium cyanoborohydride were used to prepare 509 mg (98% of theory, 70% purity) of the title compound.

### Example 387A

### 3-Cyclopropyl-1-[(3,3-difluorocyclobutyl)methyl]-6-{[(2,2-dimethoxyethyl)amino]methyl}-5-methylthieno[2,3-d]pyrimidine-2,4(1H,3H)-dione

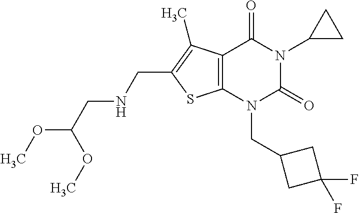 [SMILES: COC(CNCc1sc2c(c1C)c(=O)n(C1CC1)c(=O)n2CC1CC(F)(F)C1)OC]

200 mg (0.564 mmol) of the compound from Ex. 328A were dissolved in 10 ml of dichloromethane, and 92 μl (0.847 mmol) of 2,2-dimethoxyethanamine were added. The mixture was heated to 35° C. for 1 h. After cooling to RT, 378 mg (1.69 mmol) of sodium triacetoxyborohydride were added. Stirring of the mixture was continued at RT. After 2 days, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. The crude product was purified by means of MPLC (Biotage Isolera One, SNAP Ultra cartridge, 10 g of silica gel, cyclohexane/ethyl acetate 1:1). After combination of the product fractions, concentration and drying under high vacuum, 193 mg (77% of theory) of the title compound were obtained.

¹H-NMR (400 MHz, DMSO-d₆, δ/ppm): 4.40 (t, 1H), 4.02 (d, 2H), 3.81 (d, 2H), 3.26 (s, 6H), 2.74-2.55 (m, 7H), 2.31 (s, 3H), 2.29-2.20 (m, 1H), 1.05-0.94 (m, 2H), 0.73-0.59 (m, 2H).

LC/MS (Method 1, ESIpos): Rₜ=1.02 min, m/z=444.18 [M+H]⁺.

### Example 388A

### 6-{[(2,2-Dimethoxyethyl)amino]methyl}-5-methyl-3-(1-methylcyclopropyl)-1-(3,3,3-trifluoropropyl)thieno[2,3-d]pyrimidine-2,4(1H,3H)-dione

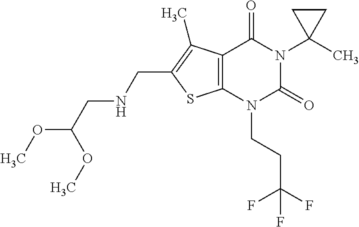

300 mg (0.833 mmol) of the compound from Ex. 64A were dissolved in 17 ml of dichloromethane, and 135 μl (1.25 mmol) of 2,2-dimethoxyethanamine were added. The mixture was heated to 35° C. for 1 h. After cooling to RT, 557 mg (2.50 mmol) of sodium triacetoxyborohydride were added. Stirring of the mixture was continued at RT. Since the conversion was still incomplete after 16 h, the mixture was heated to 35° C. for another 16 h. After cooling to RT, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. The crude product was purified by means of MPLC (Biotage Isolera One, SNAP Ultra cartridge, 25 g of silica gel, cyclohexane/ethyl acetate 2:1). After combination of the product fractions, concentration and drying under high vacuum, 300 mg (80% of theory) of the title compound were obtained.

¹H-NMR (400 MHz, DMSO-d₆, δ/ppm): 4.40 (t, 1H), 4.21-3.94 (m, 2H), 3.82 (s, 2H), 3.26 (s, 6H), 2.82-2.68 (m, 2H), 2.62 (br. d, 2H), 2.33-2.24 (broad, 1H), 2.32 (s, 3H), 1.33 (s, 3H), 1.01-0.70 (m, 4H).

LC/MS (Method 1, ESIpos): Rₜ=1.11 min, m/z=450.17 [M+H]⁺.

### Example 389A

### 1-[(3,3-Difluorocyclobutyl)methyl]-6-{[(2,2-dimethoxyethyl)amino]methyl}-5-methyl-3-(1-methylcyclopropyl)thieno[2,3-d]pyrimidine-2,4(1H,3H)-dione

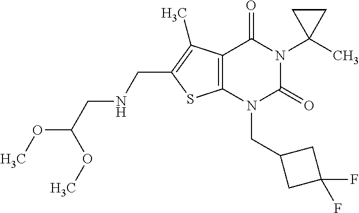 [SMILES: COC(CNCc1sc2c(c1C)c(=O)n(C1(C)CC1)c(=O)n2CC1CC(F)(F)C1)OC]

360 mg (0.977 mmol) of the compound from Ex. 335A were dissolved in 20 ml of dichloromethane, and 158 μl (154 mmol) of 2,2-dimethoxyethanamine were added. The mixture was heated to 35° C. for 1 h. After cooling to RT, 654 mg (2.93 mmol) of sodium triacetoxyborohydride were added. Stirring of the mixture was continued at RT. Since the conversion was still incomplete after 16 h, the mixture was heated to 35° C. for another 16 h. After cooling to RT, the mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. The crude product was purified by means of MPLC (Biotage Isolera One, SNAP Ultra cartridge, 25 g of silica gel, cyclohexane/ethyl acetate 1:2). After combination of the product fractions, concentration and drying under high vacuum, 324 mg (68% of theory, 95% purity) of the title compound were obtained.

¹H-NMR (400 MHz, DMSO-d₆, δ/ppm): 4.40 (t, 1H), 4.17-3.90 (m, 2H), 3.81 (s, 2H), 3.26 (s, 6H), 2.77-2.57 (m, 5H), 2.31 (s, 3H), 2.28 (br. s, 1H), 1.32 (s, 3H), 0.99-0.71 (m, 4H).

LC/MS (Method 1, ESIpos): Rₜ=1.20 min, m/z=458.19 [M+H]⁺.

### Example 390A

### 6-{[(2,2-Dimethoxyethyl)amino]methyl}-1-(2-methoxyethyl)-5-methyl-3-(1-methylcyclopropyl)thieno[2,3-d]pyrimidine-2,4(1H,3H)-dione

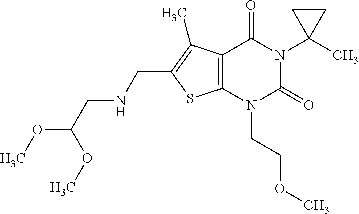

Analogously to the method described in Ex. 389A, 300 mg (0.931 mmol) of the compound from Ex. 68A, 151 μl (1.40 mmol) of 2,2-dimethoxyethanamine and 623 mg (2.79 mmol) of sodium triacetoxyborohydride were used to obtain 305 mg (74% of theory, 94% purity) of the title compound.

¹H-NMR (400 MHz, DMSO-d₆, δ/ppm): 4.40 (t, 1H), 4.14-3.86 (m, 2H), 3.80 (s, 2H), 3.68-3.57 (m, 2H), 3.26 (s, 6H), 3.24 (s, 3H), 2.61 (d, 2H), 2.31 (s, 3H), 1.33 (s, 3H), 0.97-0.74 (m, 4H).

LC/MS (Method 1, ESIpos): Rₜ=0.88 min, m/z=307.11 [M+H−C₄H₁₁NO₂]⁺.

### Example 391A

### 6-{[(2,2-Dimethoxyethyl)amino]methyl}-5-methyl-3-(2-methylcyclopropyl)-1-[2-(trifluoromethoxy)ethyl]thieno[2,3-d]pyrimidine-2,4(1H,3H)-dione (trans racemate)

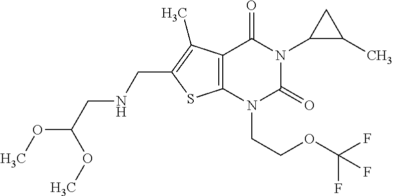

400 mg (1.02 mmol, 95% purity) of the compound from Ex. 351A were dissolved in 20 ml of dichloromethane, and 164 μl (1.52 mmol) of 2,2-dimethoxyethanamine were added. The mixture was heated to 35° C. for 1 h. After cooling to RT, 645 mg (3.04 mmol) of sodium triacetoxyborohydride were added. Stirring of the mixture was continued at RT. Since the conversion was still incomplete after 16 h, the mixture was stirred at RT for another 5 days. Thereafter, a further 55 μl (0.507 mmol) of 2,2-dimethoxyethanamine and 215 mg (1.01 mmol) of sodium triacetoxyborohydride were added, and the stirring was continued at RT for 20 h. Subsequently, the reaction mixture was diluted with dichloromethane and washed successively with saturated sodium hydrogencarbonate solution and saturated sodium chloride solution. After drying over anhydrous magnesium sulfate, the mixture was filtered and the filtrate was concentrated to dryness. The crude product was purified by means of MPLC (Biotage Isolera One, SNAP Ultra cartridge, 25 g of silica gel, cyclohexane/ethyl acetate 1:1). After combination of the product fractions, concentration and drying under high vacuum, 365 mg (74% of theory, 96% purity) of the title compound were obtained.

¹H-NMR (400 MHz, DMSO-d₆, δ/ppm): 4.40-4.36 (m, 3H), 4.25-4.06 (m, 2H), 3.81 (s, 2H), 3.25 (s, 6H), 2.60 (d, 2H), 2.31 (s, 3H), 2.27-2.21 (m, 1H), 1.15 (d, 3H), 1.02-0.92 (m, 1H), 0.88-0.78 (m, 2H).

LC/MS (Method 1, ESIpos): Rₜ=1.17 min, m/z=466.16 [M+H]⁺.

### Example 392A

### 1-({3-Cyclopropyl-1-[(3,3-difluorocyclobutyl)methyl]-5-methyl-2,4-dioxo-1,2,3,4-tetrahydrothieno[2,3-d]pyrimidin-6-yl}methyl)-1-(2,2-dimethoxyethyl)urea

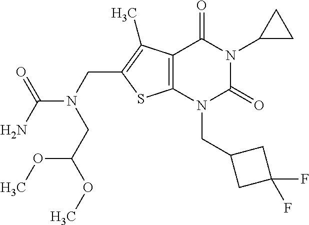 [SMILES: COC(CN(Cc1sc2c(c1C)c(=O)n(C1CC1)c(=O)n2CC1CC(F)(F)C1)C(N)=O)OC]

To a solution of 190 mg (0.428 mmol) of the compound from Ex. 387A in 5 ml of methanol were added, at RT, first 80 mg (0.985 mmol) of potassium cyanate and then 63 μl (0.728 mmol) of perchloric acid (70% in water). After 16 h, the reaction mixture was admixed with water and with aqueous sodium hydrogencarbonate solution and then extracted with ethyl acetate. The organic extract was washed with saturated sodium chloride solution, dried over anhydrous magnesium sulfate, filtered and concentrated. After the residue that remained had been dried under high vacuum, 223 mg (87% of theory, 82% purity) of the title compound were obtained, which was used for subsequent reactions without further purification.

LC/MS (Method 1, ESIpos): Rₜ=1.53 min, m/z=487.18 [M+H]⁺.

### Example 393A

### 1-(2,2-Dimethoxyethyl)-1-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methyl}urea

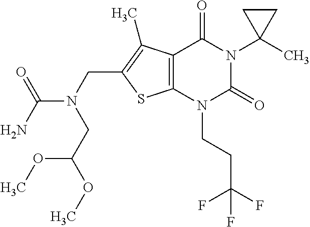 [SMILES: COC(CN(Cc1sc2c(c1C)c(=O)n(C1(C)CC1)c(=O)n2CCC(F)(F)F)C(N)=O)OC]

To a solution of 295 mg (0.656 mmol) of the compound from Ex. 388A in 7 ml of methanol were added, at RT, first 122 mg (1.51 mmol) of potassium cyanate and then 96 μl (1.12 mmol) of perchloric acid (70% in water). After 16 h, the reaction mixture was admixed with water and with saturated sodium hydrogencarbonate solution and then extracted with ethyl acetate. The organic extract was washed with saturated sodium chloride solution, dried over anhydrous magnesium sulfate, filtered and concentrated. After the residue that remained had been dried under high vacuum, 310 mg (84% of theory, 89% purity) of the title compound were obtained, which was used for subsequent reactions without further purification.

LC/MS (Method 2, ESIpos): Rₜ=0.85 min, m/z=493 [M+H]⁺.

### Example 394A

### 1-({1-[(3,3-Difluorocyclobutyl)methyl]-5-methyl-3-(1-methylcyclopropyl)-2,4-dioxo-1,2,3,4-tetrahydrothieno[2,3-d]pyrimidin-6-yl}methyl)-1-(2,2-dimethoxyethyl)urea

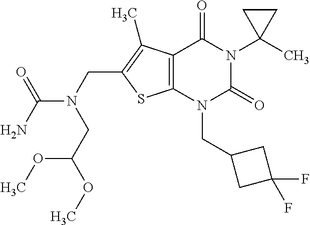 [SMILES: COC(CN(Cc1sc2c(c1C)c(=O)n(C1(C)CC1)c(=O)n2CC1CC(F)(F)C1)C(N)=O)OC]

To a solution of 320 mg (0.699 mmol) of the compound from Ex. 389A in 7 ml of methanol were added, at RT, first 130 mg (1.61 mmol) of potassium cyanate and then 103 μl (1.19 mmol) of perchloric acid (70% in water). After 16 h, the reaction mixture was admixed with water and with saturated sodium hydrogencarbonate solution and then extracted with ethyl acetate. The organic extract was washed with saturated sodium chloride solution, dried over anhydrous magnesium sulfate, filtered and concentrated. After the residue that remained had been dried under high vacuum, 350 mg (83% of theory, 83% purity) of the title compound were obtained, which was used for subsequent reactions without further purification.

LC/MS (Method 1, ESIpos): Rₜ=1.65 min, m/z=501.20 [M+H]⁺.

### Example 395A

### 1-(2,2-Dimethoxyethyl)-1-{[1-(2-methoxyethyl)-5-methyl-3-(1-methylcyclopropyl)-2,4-dioxo-1,2,3,4-tetrahydrothieno[2,3-d]pyrimidin-6-yl]methyl}urea

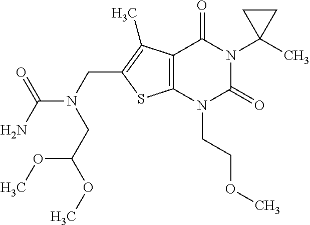 [SMILES: COCCn1c(=O)n(C2(C)CC2)c(=O)c2c(C)c(CN(CC(OC)OC)C(N)=O)sc21]

Analogously to the method described in Ex. 392A, 300 mg (0.685 mmol, 94% purity) of the compound from Ex. 390A, 128 mg (1.58 mmol) of potassium cyanate and 100 μl (1.17 mmol) of perchloric acid (70% in water) were used to obtain 310 mg (81% of theory, 81% purity) of the title compound, which was used for subsequent reactions without further purification.

LC/MS (Method 3, ESIpos): Rₜ=2.05 min, m/z=455 [M+H]⁺.

### Example 396A

### 1-(2,2-Dimethoxyethyl)-1-({5-methyl-3-(2-methylcyclopropyl)-2,4-dioxo-1-[2-(trifluormethoxy)ethyl]-1,2,3,4-tetrahydrothieno[2,3-d]pyrimidin-6-yl}methyl)urea (trans racemate)

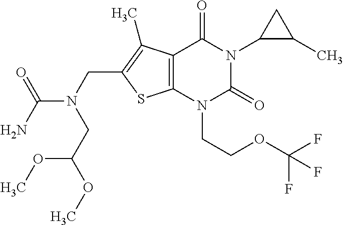 [SMILES: COC(CN(Cc1sc2c(c1C)c(=O)n(C1CC1C)c(=O)n2CCOC(F)(F)F)C(N)=O)OC]

To a solution of 445 mg (0.918 mmol, 96% purity) of the compound from Ex. 391A in 10 ml of methanol were added, at RT, first 171 mg (2.11 mmol) of potassium cyanate and then 134 μl (1.56 mmol) of perchloric acid (70% in water). Since the conversion was incomplete after 16 h, a further 86 mg (1.06 mmol) of potassium cyanate were added. After a further 23 h, the reaction mixture was admixed with water and with saturated sodium hydrogencarbonate solution and then extracted with ethyl acetate. The organic extract was washed with saturated sodium chloride solution, dried over anhydrous magnesium sulfate, filtered and concentrated. After the residue that remained had been dried under high vacuum, 542 mg (96% of theory, 83% purity) of the title compound were obtained, which was used for subsequent reactions without further purification.

LC/MS (Method 1, ESIpos): Rₜ=1.67 min, m/z=509.17 [M+H]⁺.

### Example 397A

### tert-Butyl 2-[(3-cyclopropyl-1-ethyl-5-methyl-2,4-dioxo-1,2,3,4-tetrahydrothieno[2,3-d]pyrimidin-6-yl)methylene]hydrazinecarboxylate

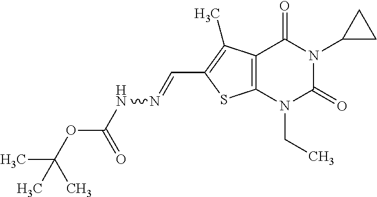 [SMILES: CCn1c(=O)n(C2CC2)c(=O)c2c(C)c(C=NNC(=O)OC(C)(C)C)sc21]

Analogously to the method described in Ex. 156A, 129 mg (0.463 mmol) of the compound from Ex. 319A and 92 mg (0.695 mmol) of tert-butyl hydrazinecarboxylate were used to prepare 177 mg (97% of theory) of the title compound.

¹H-NMR (400 MHz, DMSO-d₆, δ/ppm): 10.84 (br. s, 1H), 8.28 (s, 1H), 3.91 (q, 2H), 2.63-2.56 (m, 1H), 2.44 (s, 3H), 1.45 (s, 9H), 1.25 (t, 3H), 1.05-0.96 (m, 2H), 0.73-0.64 (m, 2H).

LC/MS (Method 1, ESIneg): Rₜ=1.84 min, m/z=391.14 [M−H]⁻.

### Example 398A

### tert-Butyl 2-[(3-cyclopropyl-5-methyl-2,4-dioxo-1-propyl-1,2,3,4-tetrahydrothieno[2,3-d]pyrimidin-6-yl)methylene]hydrazinecarboxylate

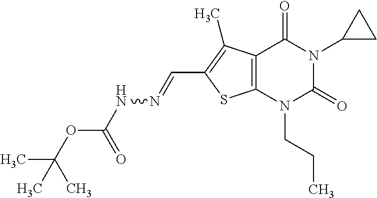 [SMILES: CCCn1c(=O)n(C2CC2)c(=O)c2c(C)c(C=NNC(=O)OC(C)(C)C)sc21]

Analogously to the method described in Ex. 156A, 133 mg (0.455 mmol) of the compound from Ex. 320A and 90 mg (0.682 mmol) of tert-butyl hydrazinecarboxylate were used to prepare 178 mg (92% of theory, 96% purity) of the title compound. The reaction time in this case was 3 h.

¹H-NMR (400 MHz, DMSO-d₆, δ/ppm): 10.84 (br. s, 1H), 8.28 (s, 1H), 3.83 (t, 2H), 2.60 (tt, 1H), 2.44 (s, 3H), 1.70 (sext, 2H), 1.45 (s, 9H), 1.04-0.97 (m, 2H), 0.92 (t, 3H), 0.73-0.64 (m, 2H).

LC/MS (Method 1, ESIneg): Rₜ=1.98 min, m/z=405.16 [M−H].

### Example 399A

### tert-Butyl 2-{[3-cyclopropyl-1-(2-fluoroethyl)-5-methyl-2,4-dioxo-1,2,3,4-tetrahydrothieno[2,3-d]pyrimidin-6-yl]methylene}hydrazinecarboxylate

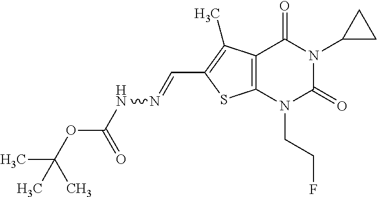 [SMILES: Cc1c(C=NNC(=O)OC(C)(C)C)sc2c1c(=O)n(C1CC1)c(=O)n2CCF]

Analogously to the method described in Ex. 156A, 80 mg (0.270 mmol) of the compound from Ex. 321A and 54 mg (0.405 mmol) of tert-butyl hydrazinecarboxylate were used to prepare 102 mg (92% of theory) of the title compound.

¹H-NMR (400 MHz, DMSO-d₆, δ/ppm): 10.84 (br. s, 1H), 8.28 (s, 1H), 4.73 (dt, 2H), 4.21 (dt, 2H), 2.65-2.57 (m, 1H), 2.44 (s, 3H), 1.45 (s, 9H), 1.06-0.95 (m, 2H), 0.76-0.66 (m, 2H).

LC/MS (Method 1, ESIneg): Rₜ=1.76 min, m/z=409.13 [M−H].

### Example 400A

### tert-Butyl 2-{[3-cyclopropyl-1-(3-fluoropropyl)-5-methyl-2,4-dioxo-1,2,3,4-tetrahydrothieno[2,3-d]pyrimidin-6-yl]methylene}hydrazinecarboxylate

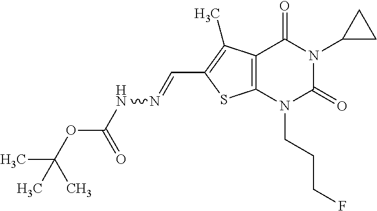 [SMILES: Cc1c(C=NNC(=O)OC(C)(C)C)sc2c1c(=O)n(C1CC1)c(=O)n2CCCF]

Analogously to the method described in Ex. 156A, 144 mg (0.455 mmol, 98% purity) of the compound from Ex. 50A and 90 mg (0.682 mmol) of tert-butyl hydrazinecarboxylate were used to prepare 169 mg (87% of theory) of the title compound. The reaction time here was 3 h.

¹H-NMR (400 MHz, DMSO-d₆, δ/ppm): 10.84 (br. s, 1H), 8.29 (s, 1H), 4.54 (dt, 2H), 3.98 (t, 2H), 2.63-2.56 (m, 1H), 2.44 (s, 3H), 2.15-1.99 (m, 2H), 1.45 (s, 9H), 1.05-0.95 (m, 2H), 0.74-0.64 (m, 2H).

LC/MS (Method 1, ESIneg): Rₜ=1.85 min, m/z=423.15 [M−H].

### Example 401A

### tert-Butyl 2-{[3-cyclopropyl-1-(4-fluorobutyl)-5-methyl-2,4-dioxo-1,2,3,4-tetrahydrothieno[2,3-d]pyrimidin-6-yl]methylene}hydrazinecarboxylate

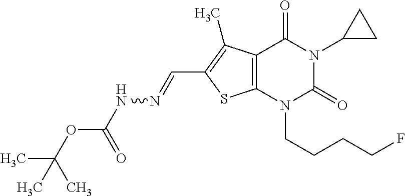 [SMILES: Cc1c(C=NNC(=O)OC(C)(C)C)sc2c1c(=O)n(C1CC1)c(=O)n2CCCCF]

Analogously to the method described in Ex. 156A, 127 mg (0.392 mmol) of the compound from Ex. 322A and 78 mg (0.587 mmol) of tert-butyl hydrazinecarboxylate were used to prepare 167 mg (94% of theory, 97% purity) of the title compound. The reaction time here was 3 h.

¹H-NMR (400 MHz, DMSO-d₆, 8/ppm): 10.84 (br. s, 1H), 8.29 (s, 1H), 4.59-4.48 (m, 1H), 4.42 (t, 1H), 3.91 (br. t, 2H), 2.63-2.56 (m, 1H), 2.44 (s, 3H), 1.84-1.63 (m, 4H), 1.45 (s, 9H), 1.05-0.95 (m, 2H), 0.73-0.65 (m, 2H).

LC/MS (Method 2, ESIneg): Rₜ=1.01 min, m/z=437 [M−H].

### Example 402A

### tert-Butyl 2-{[3-cyclopropyl-1-(2,2-difluoroethyl)-5-methyl-2,4-dioxo-1,2,3,4-tetrahydrothieno[2,3-d]pyrimidin-6-yl]methylene}hydrazinecarboxylate

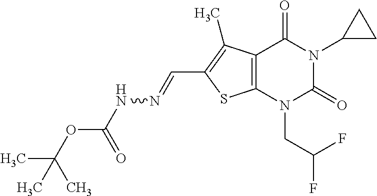 [SMILES: Cc1c(C=NNC(=O)OC(C)(C)C)sc2c1c(=O)n(C1CC1)c(=O)n2CC(F)F]

Analogously to the method described in Ex. 156A, 145 mg (0.452 mmol, 98% purity) of the compound from Ex. 323A and 90 mg (0.678 mmol) of tert-butyl hydrazinecarboxylate were used to prepare 183 mg (94% of theory) of the title compound.

¹H-NMR (400 MHz, DMSO-d₆, δ/ppm): 10.86 (br. s, 1H), 8.28 (s, 1H), 6.33 (tt, 1H), 4.34 (td, 2H), 2.66-2.57 (m, 1H), 2.44 (s, 3H), 1.45 (s, 9H), 1.07-0.95 (m, 2H), 0.77-0.66 (m, 2H).

LC/MS (Method 1, ESIneg): Rₜ=1.84 min, m/z=427.13 [M−H].

### Example 403A

### tert-Butyl 2-{[3-cyclopropyl-5-methyl-2,4-dioxo-1-(4,4,4-trifluorobutyl)-1,2,3,4-tetrahydrothieno[2,3-d]pyrimidin-6-yl]methylene}hydrazinecarboxylate

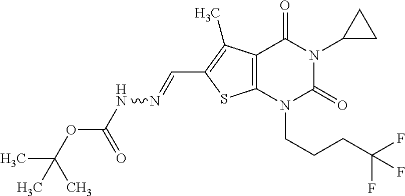 [SMILES: Cc1c(C=NNC(=O)OC(C)(C)C)sc2c1c(=O)n(C1CC1)c(=O)n2CCCC(F)(F)F]

Analogously to the method described in Ex. 156A, 168 mg (0.452 mmol, 97% purity) of the compound from Ex. 324A and 90 mg (0.678 mmol) of tert-butyl hydrazinecarboxylate were used to prepare 210 mg (94% of theory, 97% purity) of the title compound. The reaction time here was 3 h.

¹H-NMR (400 MHz, DMSO-d₆, δ/ppm): 10.84 (br. s, 1H), 8.29 (s, 1H), 3.96 (br. t, 2H), 2.62-2.55 (m, 1H), 2.48-2.35 (m, 2H), 2.44 (s, 3H), 1.90 (quin, 2H), 1.45 (s, 9H), 1.05-0.94 (m, 2H), 0.74-0.65 (m, 2H).

LC/MS (Method 1, ESIneg): Rₜ=2.07 min, m/z=473.15 [M−H].

### Example 404A

### tert-Butyl 2-({3-cyclopropyl-1-[(2,2-difluorocyclobutyl)methyl]-5-methyl-2,4-dioxo-1,2,3,4-tetrahydrothieno[2,3-d]pyrimidin-6-yl}methylene)hydrazinecarboxylate (enantiomer 1)

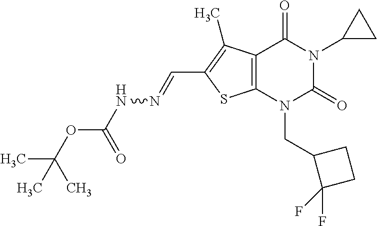 [SMILES: Cc1c(C=NNC(=O)OC(C)(C)C)sc2c1c(=O)n(C1CC1)c(=O)n2CC1CCC1(F)F]

Analogously to the method described in Ex. 156A, 130 mg (0.367 mmol) of the compound from Ex. 326A and 73 mg (0.550 mmol) of tert-butyl hydrazinecarboxylate were used to prepare 160 mg (93% of theory) of the title compound.

¹H-NMR (400 MHz, DMSO-d₆, δ/ppm): 10.86 (br. s, 1H), 8.29 (s, 1H), 4.13-3.98 (m, 2H), 2.64-2.56 (m, 2H), 2.44 (s, 3H), 2.01-1.87 (m, 1H), 1.70-1.56 (m, 1H), 1.45 (s, 9H), 1.08-0.95 (m, 2H), 0.76-0.60 (m, 2H).

LC/MS (Method 2, ESIneg): Rₜ=1.05 min, m/z=467 [M−H].

### Example 405A

### tert-Butyl 2-({3-cyclopropyl-1-[(2,2-difluorocyclobutyl)methyl]-5-methyl-2,4-dioxo-1,2,3,4-tetrahydrothieno[2,3-d]pyrimidin-6-yl}methylene)hydrazinecarboxylate (enantiomer 2)

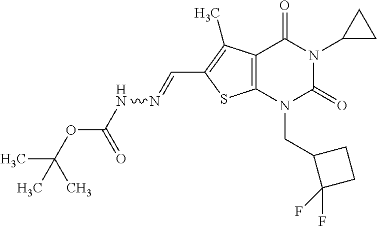 [SMILES: Cc1c(C=NNC(=O)OC(C)(C)C)sc2c1c(=O)n(C1CC1)c(=O)n2CC1CCC1(F)F]

Analogously to the method described in Ex. 156A, 116 mg (0.327 mmol) of the compound from Ex. 327A and 65 mg (0.491 mmol) of tert-butyl hydrazinecarboxylate were used to prepare 144 mg (93% of theory) of the title compound.

¹H-NMR (400 MHz, DMSO-d₆, δ/ppm): 10.85 (br. s, 1H), 8.28 (s, 1H), 4.12-3.97 (m, 2H), 2.65-2.56 (m, 1H), 2.43 (s, 3H), 2.02-1.88 (m, 1H), 1.71-1.56 (m, 1H), 1.45 (s, 9H), 1.07-0.95 (m, 2H), 0.76-0.61 (m, 2H).

LC/MS (Method 2, ESIneg): Rₜ=1.05 min, m/z=467 [M−H]⁻.

### Example 406A

### tert-Butyl 2-({3-cyclopropyl-1-[(3,3-difluorocyclobutyl)methyl]-5-methyl-2,4-dioxo-1,2,3,4-tetrahydrothieno[2,3-d]pyrimidin-6-yl}methylene)hydrazinecarboxylate

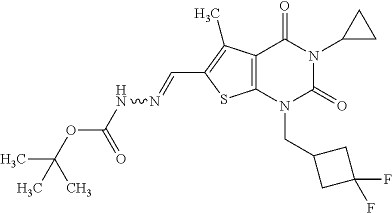 [SMILES: Cc1c(C=NNC(=O)OC(C)(C)C)sc2c1c(=O)n(C1CC1)c(=O)n2CC1CC(F)(F)C1]

To a solution of 240 mg (0.677 mmol) of the compound from Ex. 328A in 7 ml of ethanol were added first 134 mg (1.02 mmol) of tert-butyl hydrazinecarboxylate and then 3 drops of conc. hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 310 mg (97% of theory) of the title compound were obtained.

¹H-NMR (400 MHz, DMSO-d₆, δ/ppm): 10.85 (br. s, 1H), 8.28 (s, 1H), 4.06 (br. d, 2H), 2.75-2.56 (m, 4H), 2.43 (s, 3H), 1.45 (s, 9H), 1.07-0.94 (m, 2H), 0.73-0.62 (m, 2H).

LC/MS (Method 1, ESIneg): Rₜ=2.04 min, m/z=467.16 [M−H].

### Example 407A

### tert-Butyl 2-({3-cyclopropyl-1-[(2,2-difluorocyclopentyl)methyl]-5-methyl-2,4-dioxo-1,2,3,4-tetrahydrothieno[2,3-d]pyrimidin-6-yl}methylene)hydrazinecarboxylate (enantiomer 1)

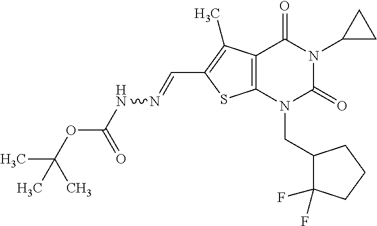 [SMILES: Cc1c(C=NNC(=O)OC(C)(C)C)sc2c1c(=O)n(C1CC1)c(=O)n2CC1CCCC1(F)F]

Analogously to the method described in Ex. 156A, 120 mg (0.326 mmol) of the compound from Ex. 330A and 65 mg (0.489 mmol) of tert-butyl hydrazinecarboxylate were used to prepare 147 mg (93% of theory) of the title compound.

¹H-NMR (400 MHz, DMSO-d₆, δ/ppm): 10.85 (br. s, 1H), 8.29 (s, 1H), 4.08-3.91 (m, 2H), 2.83-2.56 (m, 2H), 2.44 (s, 3H), 2.25-2.03 (m, 2H), 1.98-1.87 (m, 1H), 1.83-1.52 (m, 3H), 1.45 (s, 9H), 1.07-0.96 (m, 2H), 0.75-0.61 (m, 2H).

LC/MS (Method 1, ESIneg): Rₜ=2.12 min, m/z=481.17 [M−H]⁻.

### Example 408A

### tert-Butyl 2-({3-cyclopropyl-1-[(2,2-difluorocyclopentyl)methyl]-5-methyl-2,4-dioxo-1,2,3,4-tetrahydrothieno[2,3-d]pyrimidin-6-yl}methylene)hydrazinecarboxylate (enantiomer 2)

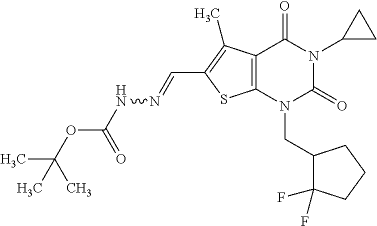 [SMILES: Cc1c(C=NNC(=O)OC(C)(C)C)sc2c1c(=O)n(C1CC1)c(=O)n2CC1CCCC1(F)F]

To a solution of 115 mg (0.312 mmol) of the compound from Ex. 331A in 4 ml of ethanol were added first 62 mg (0.468 mmol) of tert-butyl hydrazinecarboxylate and then 3 drops of conc. hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 145 mg (96% of theory) of the title compound were obtained.

¹H-NMR (400 MHz, DMSO-d₆, δ/ppm): 10.85 (br. s, 1H), 8.28 (s, 1H), 4.05-3.89 (m, 2H), 2.82-2.68 (m, 1H), 2.64-2.56 (m, 1H), 2.44 (s, 3H), 2.23-2.04 (m, 2H), 1.93 (qd, 1H), 1.81-1.52 (m, 3H), 1.45 (s, 9H), 1.06-0.95 (m, 2H), 0.74-0.61 (m, 2H).

LC/MS (Method 1, ESIneg): Rₜ=2.12 min, m/z=481.17 [M−H].

### Example 409A

### tert-Butyl 2-({3-cyclopropyl-1-[(3,3-difluorocyclopentyl)methyl]-5-methyl-2,4-dioxo-1,2,3,4-tetrahydrothieno[2,3-d]pyrimidin-6-yl}methylene)hydrazinecarboxylate (racemate)

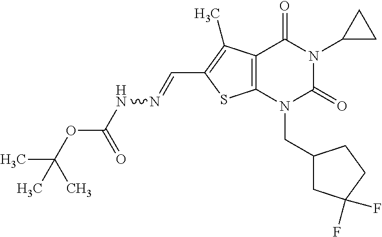 [SMILES: Cc1c(C=NNC(=O)OC(C)(C)C)sc2c1c(=O)n(C1CC1)c(=O)n2CC1CCC(F)(F)C1]

Analogously to the method described in Ex. 156A, 290 mg (0.787 mmol) of the compound from Ex. 332A and 156 mg (1.18 mmol) of tert-butyl hydrazinecarboxylate were used to prepare 350 mg (92% of theory) of the title compound.

¹H-NMR (400 MHz, DMSO-d₆, δ/ppm): 10.85 (br. s, 1H), 8.28 (s, 1H), 4.03-3.84 (m, 2H), 2.69-2.56 (m, 2H), 2.44 (s, 3H), 2.35-2.10 (m, 2H), 2.10-1.84 (m, 3H), 1.60 (dq, 1H), 1.45 (s, 9H), 1.06-0.95 (m, 2H), 0.74-0.63 (m, 2H).

LC/MS (Method 1, ESIneg): Rₜ=2.09 min, m/z=481.17 [M−H].

### Example 410A

### tert-Butyl 2-{[1-(3-cyanopropyl)-3-cyclopropyl-5-methyl-2,4-dioxo-1,2,3,4-tetrahydrothieno[2,3-d]pyrimidin-6-yl]methylene}hydrazinecarboxylate

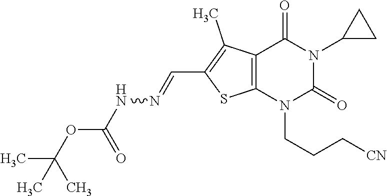 [SMILES: Cc1c(C=NNC(=O)OC(C)(C)C)sc2c1c(=O)n(C1CC1)c(=O)n2CCCC#N]

Analogously to the method described in Ex. 156A, 141 mg (0.444 mmol) of the compound from Ex. 333A and 88 mg (0.666 mmol) of tert-butyl hydrazinecarboxylate were used to prepare 184 mg (95% of theory) of the title compound.

¹H-NMR (400 MHz, DMSO-d₆, δ/ppm): 10.83 (br. s, 1H), 8.29 (s, 1H), 3.97 (t, 2H), 2.63 (t, 2H), 2.61-2.56 (m, 1H), 2.44 (s, 3H), 2.06-1.91 (m, 2H), 1.45 (s, 9H), 1.04-0.95 (m, 2H), 0.73-0.65 (m, 2H).

LC/MS (Method 1, ESIneg): Rₜ=1.72 min, m/z=430.16 [M−H]⁻.

### Example 411A

### tert-Butyl 2-[(3-cyclopropyl-5-methyl-2,4-dioxo-1-{2-[(trifluoromethyl)sulfanyl]ethyl}-1,2,3,4-tetrahydrothieno[2,3-d]pyrimidin-6-yl)methylene]hydrazinecarboxylate

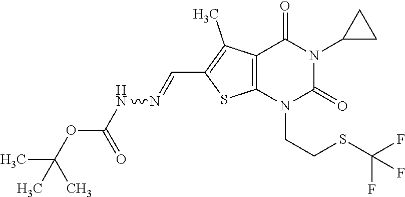 [SMILES: Cc1c(C=NNC(=O)OC(C)(C)C)sc2c1c(=O)n(C1CC1)c(=O)n2CCSC(F)(F)F]

Analogously to the method described in Ex. 156A, 170 mg (0.440 mmol, 98% purity) of the compound from Ex. 334A and 87 mg (0.660 mmol) of tert-butyl hydrazinecarboxylate were used to prepare 211 mg (97% of theory) of the title compound.

¹H-NMR (400 MHz, DMSO-d₆, δ/ppm): 10.84 (br. s, 1H), 8.29 (s, 1H), 4.15 (t, 2H), 3.34 (t, 2H), 2.60 (tt, 1H), 2.44 (s, 3H), 1.45 (s, 9H), 1.07-0.97 (m, 2H), 0.72-0.64 (m, 2H).

LC/MS (Method 1, ESIneg): Rₜ=2.13 min, m/z=491.10 [M−H].

### Example 412A

### tert-Butyl 2-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methylene)hydrazinecarboxylate (trans racemate)

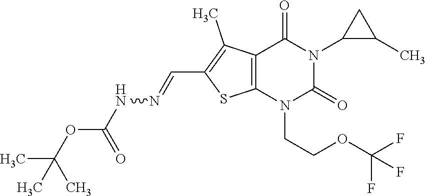 [SMILES: Cc1c(C=NNC(=O)OC(C)(C)C)sc2c1c(=O)n(C1CC1C)c(=O)n2CCOC(F)(F)F]

To a solution of 350 mg (0.930 mmol) of the compound from Ex. 351A in 10 ml of ethanol were added first 184 mg (1.40 mmol) of tert-butyl hydrazinecarboxylate and then 2 drops of conc. hydrochloric acid. After the reaction mixture had been stirred at RT for about 18 h, the majority of the ethanol was removed on a rotary evaporator. The remaining residue was diluted with 150 ml of water and neutralized by adding saturated aqueous sodium hydrogencarbonate solution. The precipitated solids were filtered off with suction, washed with a little water and dried under high vacuum. 446 mg (97% of theory) of the title compound were obtained.

¹H-NMR (400 MHz, DMSO-d₆, δ/ppm): 10.85 (br. s, 1H), 8.28 (s, 1H), 4.39 (t, 2H), 4.27-4.13 (m, 2H), 2.44 (s, 3H), 2.26 (dt, 1H), 1.45 (s, 9H), 1.15 (d, 3H), 1.06-0.93 (m, 1H), 0.89-0.79 (m, 2H).

LC/MS (Method 1, ESIneg): Rₜ=2.13 min, m/z=489.14 [M−H].

### Example 413A

### tert-Butyl 2-({1-(2-methoxyethyl)-5-methyl-2,4-dioxo-3-[2-(trifluoromethyl)cyclopropyl]-1,2,3,4-tetrahydrothieno[2,3-d]pyrimidin-6-yl}methylene)hydrazinecarboxylate (trans racemate)

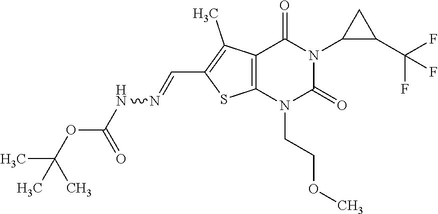 [SMILES: COCCn1c(=O)n(C2CC2C(F)(F)F)c(=O)c2c(C)c(C=NNC(=O)OC(C)(C)C)sc21]

To a solution of 300 mg (0.797 mmol) of the compound from Ex. 357A in 8 ml of ethanol were added first 158 mg (1.20 mmol) of tert-butyl hydrazinecarboxylate and then 3 drops of conc. hydrochloric acid. After the reaction mixture had been stirred at RT for about 18 h, it was diluted with 150 ml of water and neutralized by adding saturated aqueous sodium hydrogencarbonate solution. The mixture was extracted three time with about 50 ml each time of ethyl acetate. The organic extract was washed successively with water and saturated aqueous sodium chloride solution. After drying over anhydrous magnesium sulfate, the mixture was filtered and concentrated. Drying under high vacuum gave 381 mg (97% of theory) of the title compound.

¹H-NMR (400 MHz, DMSO-d₆, δ/ppm): 10.85 (br. s, 1H), 8.28 (s, 1H), 4.12-3.93 (m, 2H), 3.64 (t, 2H), 3.26 (s, 3H), 3.03-2.92 (m, 1H), 2.43 (s, 3H), 2.25 (dtd, 1H), 1.50 (q, 1H), 1.45 (s, 9H), 1.38-1.27 (m, 1H).

LC/MS (Method 1, ESIneg): Rₜ=2.00 min, m/z=489.14 [M−H].

### Example 414A

### tert-Butyl 2-{[3-(2-ethylcyclopropyl)-1-(2-methoxyethyl)-5-methyl-2,4-dioxo-1,2,3,4-tetrahydrothieno[2,3-d]pyrimidin-6-yl]methylene}hydrazinecarboxylate (trans racemate)

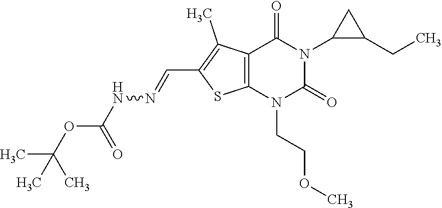 [SMILES: CCC1CC1n1c(=O)c2c(C)c(C=NNC(=O)OC(C)(C)C)sc2n(CCOC)c1=O]

Analogously to the method described in Ex. 413A, 275 mg (0.817 mmol) of the compound from Ex. 358A and 162 mg (1.23 mmol) of tert-butyl hydrazinecarboxylate were used to obtain 402 mg (quant.) of the title compound.

¹H-NMR (400 MHz, DMSO-d₆, δ/ppm): 10.83 (broad, 1H), 8.27 (s, 1H), 4.08-3.97 (m, 2H), 3.63 (t, 2H), 3.25 (s, 3H), 2.43 (s, 3H), 2.31 (dt, 1H), 1.69-1.54 (m, 1H), 1.45 (s, 9H), 1.31-1.16 (m, 1H), 1.08-0.94 (m, 1H), 1.00 (t, 3H), 0.89-0.74 (m, 2H).

LC/MS (Method 2, ESIneg): Rₜ=1.09 min, m/z=449 [M−H].

### Example 415A

### tert-Butyl 2-{[3-(2-methoxycyclopropyl)-1-(2-methoxyethyl)-5-methyl-2,4-dioxo-1,2,3,4-tetrahydrothieno[2,3-d]pyrimidin-6-yl]methylene}hydrazinecarboxylate (trans racemate)

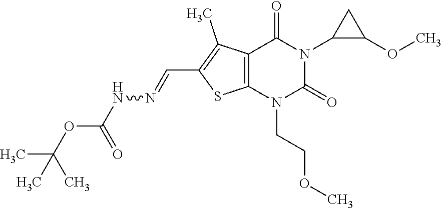 [SMILES: COCCn1c(=O)n(C2CC2OC)c(=O)c2c(C)c(C=NNC(=O)OC(C)(C)C)sc21]

Analogously to the method described in Ex. 413A, 350 mg (1.03 mmol) of the compound from Ex. 359A and 205 mg (1.55 mmol) of tert-butyl hydrazinecarboxylate were used to obtain 438 mg (93% of theory) of the title compound.

¹H-NMR (400 MHz, DMSO-d₆, δ/ppm): 10.84 (br. s, 1H), 8.28 (s, 1H), 4.14-3.96 (m, 2H), 3.67-3.59 (m, 2H), 3.47-3.38 (m, 1H), 3.24 (s, 3H), 3.23 (s, 3H), 2.61 (dt, 1H), 2.43 (s, 3H), 1.45 (s, 9H), 1.31 (td, 1H), 0.91 (ddd, 1H).

LC/MS (Method 1, ESIpos): Rₜ=1.66 min, m/z=453.18 [M+H]⁺.

### Example 416A

### tert-Butyl 2-[(3-cyclopropyl-1-ethyl-5-methyl-2,4-dioxo-1,2,3,4-tetrahydrothieno[2,3-d]pyrimidin-6-yl)methyl]hydrazinecarboxylate

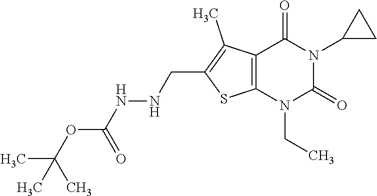 [SMILES: CCn1c(=O)n(C2CC2)c(=O)c2c(C)c(CNNC(=O)OC(C)(C)C)sc21]

Analogously to the method described in Ex. 200A, 177 mg (0.451 mmol) of the compound from Ex. 397A and a total of 213 mg (3.38 mmol) of sodium cyanoborohydride were used to prepare 155 mg (87% of theory) of the title compound.

¹H-NMR (400 MHz, DMSO-d₆, δ/ppm): 8.24 (br. s, 1H), 4.99 (br. d, 1H), 3.96 (br. d, 2H), 3.87 (q, 2H), 2.63-2.56 (m, 1H), 2.31 (s, 3H), 1.38 (s, 9H), 1.24 (t, 3H), 1.05-0.95 (m, 2H), 0.72-0.62 (m, 2H).

LC/MS (Method 1, ESIneg): Rₜ=1.69 min, m/z=439.17 [M−H+HCOOH].

### Example 417A

### tert-Butyl 2-[(3-cyclopropyl-5-methyl-2,4-dioxo-1-propyl-1,2,3,4-tetrahydrothieno[2,3-d]pyrimidin-6-yl)methyl]hydrazinecarboxylate

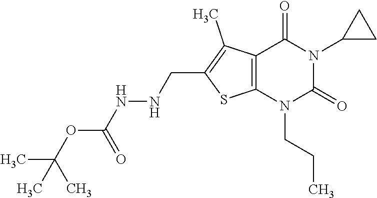 [SMILES: CCCn1c(=O)n(C2CC2)c(=O)c2c(C)c(CNNC(=O)OC(C)(C)C)sc21]

Analogously to the method described in Ex. 200A, 178 mg (0.438 mmol) of the compound from Ex. 398A and a total of 206 mg (3.28 mmol) of sodium cyanoborohydride were used to prepare 146 mg (77% of theory, 95% purity) of the title compound.

¹H-NMR (500 MHz, DMSO-d₆, δ/ppm): 8.24 (br. s, 1H), 4.99 (br. d, 1H), 3.96 (br. d, 2H), 3.84-3.74 (m, 2H), 2.62-2.56 (m, 1H), 2.31 (s, 3H), 1.70 (sext, 2H), 1.38 (s, 9H), 1.04-0.96 (m, 2H), 0.91 (t, 3H), 0.71-0.61 (m, 2H).

LC/MS (Method 1, ESIneg): Rₜ=1.83 min, m/z=453.18 [M−H+HCOOH].

### Example 418A

### tert-Butyl 2-{[3-cyclopropyl-1-(2-fluoroethyl)-5-methyl-2,4-dioxo-1,2,3,4-tetrahydrothieno[2,3-d]pyrimidin-6-yl]methyl}hydrazinecarboxylate

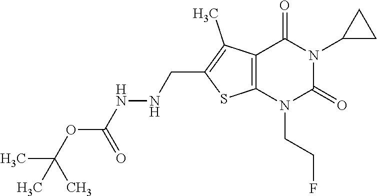 [SMILES: Cc1c(CNNC(=O)OC(C)(C)C)sc2c1c(=O)n(C1CC1)c(=O)n2CCF]

Analogously to the method described in Ex. 200A, 102 mg (0.248 mmol) of the compound from Ex. 399A and a total of 117 mg (1.86 mmol) of sodium cyanoborohydride were used to prepare 88 mg (85% of theory) of the title compound.

¹H-NMR (400 MHz, DMSO-d₆, δ/ppm): 8.24 (br. s, 1H), 5.02-4.91 (m, 1H), 4.72 (dt, 2H), 4.15 (dt, 2H), 3.96 (br. d, 2H), 2.65-2.57 (m, 1H), 2.31 (s, 3H), 1.38 (s, 9H), 1.06-0.95 (m, 2H), 0.73-0.63 (m, 2H).

LC/MS (Method 1, ESIneg): Rₜ=1.62 min, m/z=457.16 [M−H+HCOOH].

### Example 419A

### tert-Butyl 2-{[3-cyclopropyl-1-(3-fluoropropyl)-5-methyl-2,4-dioxo-1,2,3,4-tetrahydrothieno[2,3-d]pyrimidin-6-yl]methyl}hydrazinecarboxylate

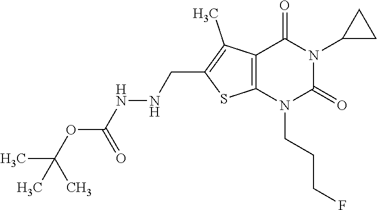 [SMILES: Cc1c(CNNC(=O)OC(C)(C)C)sc2c1c(=O)n(C1CC1)c(=O)n2CCCF]

Analogously to the method described in Ex. 200A, 169 mg (0.398 mmol) of the compound from Ex. 400A and a total of 188 mg (2.99 mmol) of sodium cyanoborohydride were used to prepare 176 mg (88% of theory, 85% purity) of the title compound.

¹H-NMR (400 MHz, DMSO-d₆, δ/ppm): 8.24 (br. s, 1H), 4.99 (br. d, 1H), 4.65-4.42 (m, 2H), 4.01-3.90 (m, 4H), 2.63-2.56 (m, 1H), 2.31 (s, 3H), 2.14-1.99 (m, 2H), 1.38 (s, 9H), 1.04-0.96 (m, 2H), 0.72-0.62 (m, 2H).

LC/MS (Method 1, ESIneg): Rₜ=1.70 min, m/z=471.17 [M−H+HCOOH]⁻.

### Example 420A

### tert-Butyl 2-{[3-cyclopropyl-1-(4-fluorobutyl)-5-methyl-2,4-dioxo-1,2,3,4-tetrahydrothieno[2,3-d]pyrimidin-6-yl]methyl}hydrazinecarboxylate

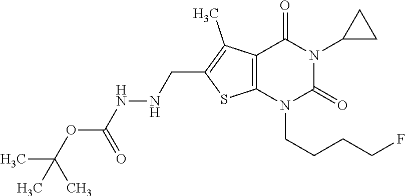 [SMILES: Cc1c(CNNC(=O)OC(C)(C)C)sc2c1c(=O)n(C1CC1)c(=O)n2CCCCF]

Analogously to the method described in Ex. 200A, 182 mg (0.403 mmol, 97% purity) of the compound from Ex. 401A and a total of 190 mg (3.02 mmol) of sodium cyanoborohydride were used to prepare 143 mg (72% of theory, 90% purity) of the title compound.

¹H-NMR (400 MHz, DMSO-d₆, δ/ppm): 8.24 (br. s, 1H), 4.99 (br. d, 1H), 4.47 (dt, 2H), 3.96 (br. d, 2H), 3.88 (br. t, 2H), 2.64-2.56 (m, 1H), 2.31 (s, 3H), 1.83-1.61 (m, 4H), 1.38 (s, 9H), 1.05-0.95 (m, 2H), 0.71-0.61 (m, 2H).

LC/MS (Method 1, ESIneg): Rₜ=1.80 min, m/z=485.19 [M−H+HCOOH].

### Example 421A

### tert-Butyl 2-{[3-cyclopropyl-1-(2,2-difluoroethyl)-5-methyl-2,4-dioxo-1,2,3,4-tetrahydrothieno[2,3-d]pyrimidin-6-yl]methyl}hydrazinecarboxylate

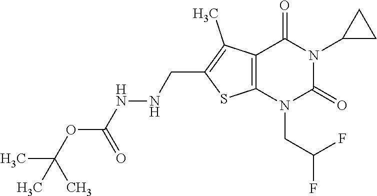 [SMILES: Cc1c(CNNC(=O)OC(C)(C)C)sc2c1c(=O)n(C1CC1)c(=O)n2CC(F)F]

Analogously to the method described in Ex. 200A, 210 mg (0.480 mmol, 98% purity) of the compound from Ex. 402A and a total of 226 mg (3.60 mmol) of sodium cyanoborohydride were used to prepare 130 mg (50% of theory, 81% purity) of the title compound.

¹H-NMR (400 MHz, DMSO-d₆, δ/ppm): 8.24 (br. s, 1H), 6.33 (tt, 1H), 4.99 (br. d, 1H), 4.27 (td, 2H), 3.97 (br. d, 2H), 2.62 (tt, 1H), 2.31 (s, 3H), 1.38 (s, 9H), 1.06-0.96 (m, 2H), 0.75-0.64 (m, 2H).

LC/MS (Method 2, ESIneg): Rₜ=0.91 min, m/z=475 [M−H+HCOOH].

### Example 422A

### tert-Butyl 2-{[3-cyclopropyl-5-methyl-2,4-dioxo-1-(4,4,4-trifluorobutyl)-1,2,3,4-tetrahydrothieno[2,3-d]pyrimidin-6-yl]methyl}hydrazinecarboxylate

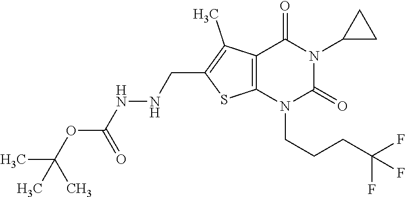 [SMILES: Cc1c(CNNC(=O)OC(C)(C)C)sc2c1c(=O)n(C1CC1)c(=O)n2CCCC(F)(F)F]

Analogously to the method described in Ex. 200A, 210 mg (0.434 mmol, 98% purity) of the compound from Ex. 403A and a total of 204 mg (3.25 mmol) of sodium cyanoborohydride were used to prepare 188 mg (77% of theory, 85% purity) of the title compound.

¹H-NMR (400 MHz, DMSO-d₆, δ/ppm): 8.23 (br. s, 1H), 4.99 (br. d, 1H), 3.97 (br. d, 2H), 3.92 (t, 2H), 2.62-2.56 (m, 1H), 2.45-2.33 (m, 2H), 2.31 (s, 3H), 1.96-1.82 (m, 2H), 1.38 (s, 9H), 1.04-0.95 (m, 2H), 0.72-0.62 (m, 2H).

LC/MS (Method 2, ESIneg): Rₜ=1.02 min, m/z=521 [M−H+HCOOH].

### Example 423A

### tert-Butyl 2-({3-cyclopropyl-1-[(2,2-difluorocyclobutyl)methyl]-5-methyl-2,4-dioxo-1,2,3,4-tetrahydrothieno[2,3-d]pyrimidin-6-yl}methyl)hydrazinecarboxylate (enantiomer 1)

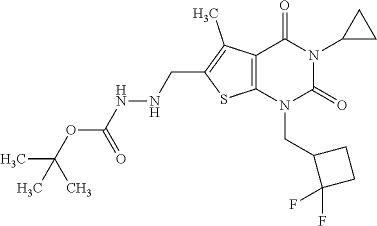 [SMILES: Cc1c(CNNC(=O)OC(C)(C)C)sc2c1c(=O)n(C1CC1)c(=O)n2CC1CCC1(F)F]

Analogously to the method described in Ex. 200A, 158 mg (0.337 mmol) of the compound from Ex. 404A and a total of 159 mg (2.53 mmol) of sodium cyanoborohydride were used to prepare 143 mg (58% of theory, 65% of theory) of the title compound.

LC/MS (Method 1, ESIneg): Rₜ=1.93 min, m/z=515.18 [M−H+HCOOH].

### Example 424A

### tert-Butyl 2-({3-cyclopropyl-1-[(2,2-difluorocyclobutyl)methyl]-5-methyl-2,4-dioxo-1,2,3,4-tetrahydrothieno[2,3-d]pyrimidin-6-yl}methyl)hydrazinecarboxylate (enantiomer 2)

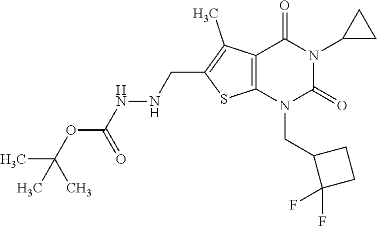 [SMILES: Cc1c(CNNC(=O)OC(C)(C)C)sc2c1c(=O)n(C1CC1)c(=O)n2CC1CCC1(F)F]

Analogously to the method described in Ex. 200A, 140 mg (0.299 mmol) of the compound from Ex. 405A and a total of 141 mg (2.24 mmol) of sodium cyanoborohydride were used to prepare 103 mg (55% of theory, 76% purity) of the title compound.

LC/MS (Method 1, ESIneg): Rₜ=1.93 min, m/z=515.18 [M−H+HCOOH].

### Example 425A

### tert-Butyl 2-({3-cyclopropyl-1-[(3,3-difluorocyclobutyl)methyl]-5-methyl-2,4-dioxo-1,2,3,4-tetrahydrothieno[2,3-d]pyrimidin-6-yl}methyl)hydrazinecarboxylate

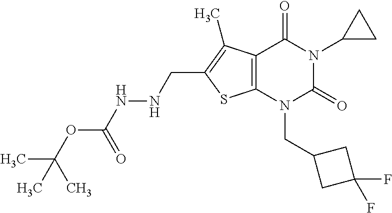 [SMILES: Cc1c(CNNC(=O)OC(C)(C)C)sc2c1c(=O)n(C1CC1)c(=O)n2CC1CC(F)(F)C1]

To a solution of 305 mg (0.651 mmol) of the compound from Ex. 406A in 12 ml of methanol were added 205 mg (3.26 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 102 mg (1.63 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 25 g of silica gel, eluent: cyclohexane/ethyl acetate 2:1). The product fractions were combined and concentrated. After drying under high vacuum, 236 mg (73% of theory, 95% purity) of the title compound were obtained.

¹H-NMR (400 MHz, DMSO-d₆, δ/ppm): 8.24 (br. s, 1H), 5.02 (br. d, 1H), 4.02 (br. d, 2H), 3.96 (br. d, 2H), 2.75-2.56 (m, 5H), 2.30 (s, 3H), 1.38 (s, 9H), 1.09-0.92 (m, 2H), 0.74-0.58 (m, 2H).

LC/MS (Method 1, ESIneg): Rₜ=1.89 min, m/z=515.18 [M−H+HCOOH]⁻.

### Example 426A

### tert-Butyl 2-({3-cyclopropyl-1-[(2,2-difluorocyclopentyl)methyl]-5-methyl-2,4-dioxo-1,2,3,4-tetrahydrothieno[2,3-d]pyrimidin-6-yl}methyl)hydrazinecarboxylate (enantiomer 1)

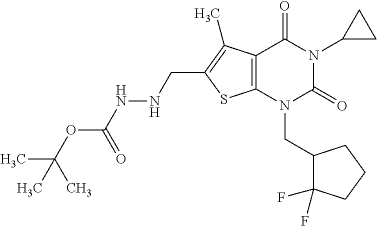 [SMILES: Cc1c(CNNC(=O)OC(C)(C)C)sc2c1c(=O)n(C1CC1)c(=O)n2CC1CCCC1(F)F]

Analogously to the method described in Ex. 200A, 145 mg (0.300 mmol) of the compound from Ex. 407A and a total of 142 mg (2.25 mmol) of sodium cyanoborohydride were used to prepare 122 mg (72% of theory, 87% purity) of the title compound.

¹H-NMR (400 MHz, DMSO-d₆, δ/ppm): 8.24 (br. s, 1H), 5.00 (br. d, 1H), 4.07-3.85 (m, 4H), 2.82-2.67 (m, 1H), 2.63-2.56 (m, 1H), 2.31 (s, 3H), 2.23-2.04 (m, 2H), 1.94-1.84 (m, 1H), 1.83-1.51 (m, 3H), 1.38 (s, 9H), 1.06-0.94 (m, 2H), 0.72-0.59 (m, 2H).

LC/MS (Method 1, ESIneg): Rₜ=1.98 min, m/z=529.19 [M−H+HCOOH].

### Example 427A

### tert-Butyl 2-({3-cyclopropyl-1-[(2,2-difluorocyclopentyl)methyl]-5-methyl-2,4-dioxo-1,2,3,4-tetrahydrothieno[2,3-d]pyrimidin-6-yl}methyl)hydrazinecarboxylate (enantiomer 2)

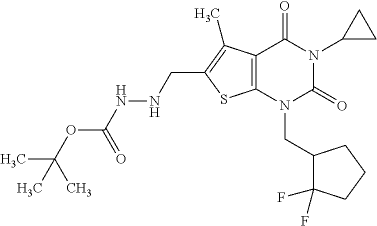 [SMILES: Cc1c(CNNC(=O)OC(C)(C)C)sc2c1c(=O)n(C1CC1)c(=O)n2CC1CCCC1(F)F]

To a solution of 142 mg (0.294 mmol) of the compound from Ex. 408A in 6 ml of methanol were added 92 mg (1.47 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 46 mg (0.736 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 25 g of silica gel, eluent: cyclohexane/ethyl acetate 1:1). The product fractions were combined and concentrated. After drying under high vacuum, 130 mg (80% of theory, 88% purity) of the title compound were obtained.

¹H-NMR (400 MHz, DMSO-d₆, δ/ppm): 8.24 (br. s, 1H), 5.00 (br. d, 1H), 4.08-3.84 (m, 4H), 2.82-2.68 (m, 1H), 2.64-2.56 (m, 1H), 2.31 (s, 3H), 2.23-2.05 (m, 2H), 1.95-1.84 (m, 1H), 1.82-1.52 (m, 3H), 1.38 (s, 9H), 1.07-0.93 (m, 2H), 0.73-0.59 (m, 2H).

LC/MS (Method 1, ESIneg): Rₜ=1.98 min, m/z=529.19 [M−H+HCOOH].

### Example 428A

### tert-Butyl 2-({3-cyclopropyl-1-[(3,3-difluorocyclopentyl)methyl]-5-methyl-2,4-dioxo-1,2,3,4-tetrahydrothieno[2,3-d]pyrimidin-6-yl}methyl)hydrazinecarboxylate (racemate)

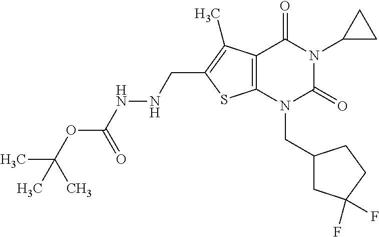 [SMILES: Cc1c(CNNC(=O)OC(C)(C)C)sc2c1c(=O)n(C1CC1)c(=O)n2CC1CCC(F)(F)C1]

Analogously to the method described in Ex. 200A, 340 mg (0.705 mmol) of the compound from Ex. 409A and a total of 332 mg (5.28 mmol) of sodium cyanoborohydride were used to prepare 241 mg (67% of theory, 95% purity) of the title compound.

¹H-NMR (400 MHz, DMSO-d₆, δ/ppm): 8.24 (br. s, 1H), 5.00 (br. d, 1H), 3.96 (br. d, 2H), 3.90 (t, 2H), 2.70-2.56 (m, 2H), 2.39-2.11 (m, 2H), 2.31 (s, 3H), 2.10-1.81 (m, 3H), 1.68-1.52 (m, 1H), 1.38 (s, 9H), 1.07-0.93 (m, 2H), 0.74-0.59 (m, 2H).

LC/MS (Method 1, ESIneg): Rₜ=1.98 min, m/z=529.19 [M−H+HCOOH]⁻.

### Example 429A

### tert-Butyl 2-{[1-(3-cyanopropyl)-3-cyclopropyl-5-methyl-2,4-dioxo-1,2,3,4-tetrahydrothieno[2,3-d]pyrimidin-6-yl]methyl}hydrazinecarboxylate

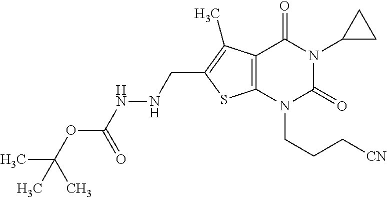 [SMILES: Cc1c(CNNC(=O)OC(C)(C)C)sc2c1c(=O)n(C1CC1)c(=O)n2CCCC#N]

Analogously to the method described in Ex. 200A, 184 mg (0.426 mmol) of the compound from Ex. 410A and a total of 201 mg (3.20 mmol) of sodium cyanoborohydride were used to prepare 125 mg (67% of theory) of the title compound.

¹H-NMR (400 MHz, DMSO-d₆, δ/ppm): 8.25 (br. s, 1H), 5.01 (br. d, 1H), 4.00-3.90 (m, 4H), 2.66-2.55 (m, 3H), 2.31 (s, 3H), 2.04-1.91 (m, 2H), 1.38 (s, 9H), 1.06-0.94 (m, 2H), 0.74-0.63 (m, 2H).

LC/MS (Method 1, ESIneg): Rₜ=1.57 min, m/z=478.18 [M−H+HCOOH].

### Example 430A

### tert-Butyl 2-[(3-cyclopropyl-5-methyl-2,4-dioxo-1-{2-[(trifluoromethyl)sulfanyl]ethyl}-1,2,3,4-tetrahydrothieno[2,3-d]pyrimidin-6-yl)methyl]hydrazinecarboxylate

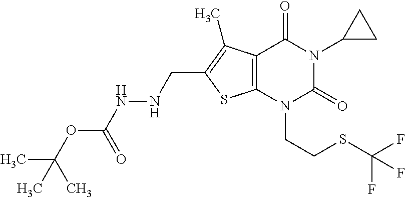 [SMILES: Cc1c(CNNC(=O)OC(C)(C)C)sc2c1c(=O)n(C1CC1)c(=O)n2CCSC(F)(F)F]

Analogously to the method described in Ex. 200A, 210 mg (0.418 mmol, 98% purity) of the compound from Ex. 411A and a total of 197 mg (3.13 mmol) of sodium cyanoborohydride were used to prepare 181 mg (78% of theory, 90% purity) of the title compound.

¹H-NMR (500 MHz, DMSO-d₆, δ/ppm): 8.23 (br. s, 1H), 5.00 (br. d, 1H), 4.11 (t, 2H), 3.97 (br. d, 2H), 3.34 (t, 2H), 2.62-2.56 (m, 1H), 2.31 (s, 3H), 1.38 (s, 9H), 1.06-0.96 (m, 2H), 0.72-0.63 (m, 2H).

LC/MS (Method 6, ESIneg): Rₜ=1.38 min, m/z=539 [M−H+HCOOH].

### Example 431A

### tert-Butyl 2-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)hydrazinecarboxylate (trans racemate)

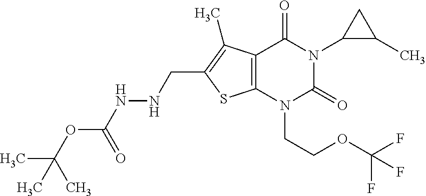 [SMILES: Cc1c(CNNC(=O)OC(C)(C)C)sc2c1c(=O)n(C1CC1C)c(=O)n2CCOC(F)(F)F]

To a solution of 444 mg (0.905 mmol) of the compound from Ex. 412A in 18 ml of methanol were added 284 mg (4.53 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 142 mg (2.26 mmol) of sodium cyanoborohydride were added. Over the entire reaction time, by addition of further acetic acid, the pH was constantly regulated such that the indicator colour just remained yellow. After a total of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 25 g of silica gel, eluent: cyclohexane/ethyl acetate 1:1). The product fractions were combined and concentrated. After drying under high vacuum, 375 mg (79% of theory, 95% purity) of the title compound were obtained.

¹H-NMR (400 MHz, DMSO-d₆, δ/ppm): 8.23 (br. s, 1H), 4.98 (br. d, 1H), 4.43-4.31 (m, 2H), 4.22-4.07 (m, 2H), 3.96 (br. d, 2H), 2.31 (s, 3H), 2.28-2.21 (m, 1H), 1.38 (s, 9H), 1.15 (d, 3H), 1.04-0.91 (m, 1H), 0.88-0.76 (m, 2H).

LC/MS (Method 2, ESIneg): Rₜ=1.07 min, m/z=537 [M−H+HCOOH].

### Example 432A

### tert-Butyl 2-({1-(2-methoxyethyl)-5-methyl-2,4-dioxo-3-[2-(trifluoromethyl)cyclopropyl]-1,2,3,4-tetrahydrothieno[2,3-d]pyrimidin-6-yl}methyl)hydrazinecarboxylate (trans racemate)

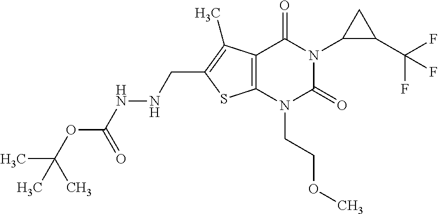 [SMILES: COCCn1c(=O)n(C2CC2C(F)(F)F)c(=O)c2c(C)c(CNNC(=O)OC(C)(C)C)sc21]

Analogously to the method described in Ex. 200A, 381 mg (0.777 mmol) of the compound from Ex. 413A and 249 mg (3.88 mmol) of sodium cyanoborohydride were used to prepare 311 mg (79% of theory, 97% purity) of the title compound. Differences here were that there was no further addition of sodium cyanoborohydride after 1 h and the reaction time was 4 h.

¹H-NMR (400 MHz, DMSO-d₆, δ/ppm): 8.24 (br. s, 1H), 4.98 (br. d, 1H), 4.08-3.88 (m, 4H), 3.63 (t, 2H), 3.25 (s, 3H), 3.02-2.94 (m, 1H), 2.31 (s, 3H), 2.24 (dtd, 1H), 1.55-1.44 (m, 1H), 1.41-1.29 (m, 1H), 1.39 (s, 9H).

LC/MS (Method 1, ESIneg): Rₜ=1.90 min, m/z=491.16 [M−H+HCOOH].

### Example 433A

### tert-Butyl 2-{[3-(2-ethylcyclopropyl)-1-(2-methoxyethyl)-5-methyl-2,4-dioxo-1,2,3,4-tetrahydrothieno[2,3-d]pyrimidin-6-yl]methyl}hydrazinecarboxylate (trans racemate)

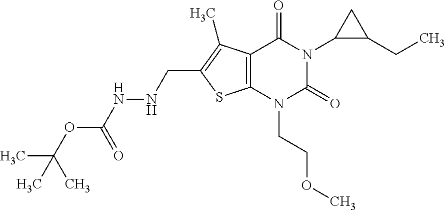 [SMILES: CCC1CC1n1c(=O)c2c(C)c(CNNC(=O)OC(C)(C)C)sc2n(CCOC)c1=O]

Analogously to the method described in Ex. 200A, 401 mg (0.890 mmol) of the compound from Ex. 414A and 285 mg (4.45 mmol) of sodium cyanoborohydride were used to prepare 357 mg (84% of theory, 95% purity) of the title compound. Differences here were that there was no further addition of sodium cyanoborohydride after 1 h and the reaction time was 4 h.

¹H-NMR (400 MHz, DMSO-d₆, δ/ppm): 8.34-8.14 (m, 1H), 4.96 (br. d, 1H), 4.05-3.89 (m, 4H), 3.62 (t, 2H), 3.25 (s, 3H), 2.35-2.24 (m, 1H), 2.30 (s, 3H), 1.69-1.56 (m, 1H), 1.38 (s, 9H), 1.22 (dt, 1H), 1.04-0.96 (m, 1H), 0.99 (t, 3H), 0.88-0.72 (m, 2H).

LC/MS (Method 2, ESIneg): Rₜ=1.03 min, m/z=497 [M−H+HCOOH].

### Example 434A

### tert-Butyl 2-{[3-(2-methoxycyclopropyl)-1-(2-methoxyethyl)-5-methyl-2,4-dioxo-1,2,3,4-tetrahydrothieno[2,3-d]pyrimidin-6-yl]methyl}hydrazinecarboxylate (trans racemate)

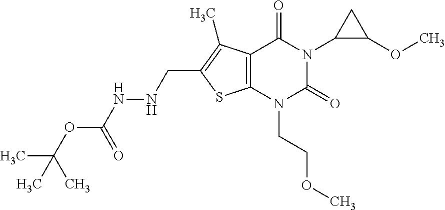 [SMILES: COCCn1c(=O)n(C2CC2OC)c(=O)c2c(C)c(CNNC(=O)OC(C)(C)C)sc21]

Analogously to the method described in Ex. 200A, 438 mg (0.958 mmol) of the compound from Ex. 415A and 307 mg (4.79 mmol) of sodium cyanoborohydride were used to prepare 305 mg (70% of theory) of the title compound. Differences here were that there was no further addition of sodium cyanoborohydride after 1 h and the reaction time was 4 h.

¹H-NMR (400 MHz, DMSO-d₆, δ/ppm): 8.26 (broad, 1H), 4.98 (br. d, 1H), 4.10-3.89 (m, 4H), 3.66-3.59 (m, 2H), 3.45-3.37 (m, 1H), 3.24 (s, 3H), 3.23 (s, 3H), 2.61 (dt, 1H), 2.30 (s, 3H), 1.39 (s, 9H), 1.30 (td, 1H), 0.90 (ddd, 1H).

LC/MS (Method 1, ESIneg): Rₜ=1.53 min, m/z=499.19 [M−H+HCOOH].

### Example 435A

### tert-Butyl 2-carbamoyl-2-[(3-cyclopropyl-1-ethyl-5-methyl-2,4-dioxo-1,2,3,4-tetrahydrothieno[2,3-d]pyrimidin-6-yl)methyl]hydrazinecarboxylate

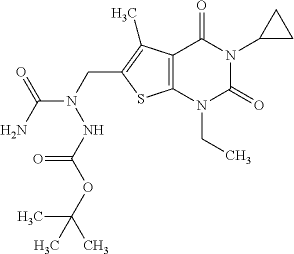 [SMILES: CCn1c(=O)n(C2CC2)c(=O)c2c(C)c(CN(NC(=O)OC(C)(C)C)C(N)=O)sc21]

Analogously to the method described in Ex. 244A, 155 mg (0.393 mmol) of the compound from Ex. 416A and 105 μl (0.786 mmol) of trimethylsilyl isocyanate were used to prepare 170 mg (71% of theory, 72% purity) of the title compound. The reaction time here was about 16 h.

LC/MS (Method 1, ESIneg): Rₜ=1.38 min, m/z=436.17 [M−H].

### Example 436A

### tert-Butyl 2-carbamoyl-2-[(3-cyclopropyl-5-methyl-2,4-dioxo-1-propyl-1,2,3,4-tetrahydrothieno[2,3-d]pyrimidin-6-yl)methyl]hydrazinecarboxylate

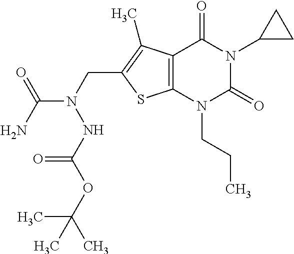 [SMILES: CCCn1c(=O)n(C2CC2)c(=O)c2c(C)c(CN(NC(=O)OC(C)(C)C)C(N)=O)sc21]

Analogously to the method described in Ex. 244A, 145 mg (0.337 mmol, 95% purity) of the compound from Ex. 417A and 90 μl (0.674 mmol) of trimethylsilyl isocyanate were used to prepare 108 mg (67% of theory, 95% purity) of the title compound. The reaction time here was about 40 h.

¹H-NMR (400 MHz, DMSO-d₆, δ/ppm): 8.90 (br. s, 1H), 6.18 (br. s, 2H), 3.79 (br. t, 2H), 2.64-2.57 (m, 1H), 2.32 (s, 3H), 1.68 (sext, 2H), 1.38 (br. s, 9H), 1.06-0.97 (m, 2H), 0.90 (t, 3H), 0.66-0.61 (m, 2H).

LC/MS (Method 1, ESIneg): Rₜ=1.50 min, m/z=450.18 [M−H].

### Example 437A

### tert-Butyl 2-carbamoyl-2-{[3-cyclopropyl-1-(2-fluoroethyl)-5-methyl-2,4-dioxo-1,2,3,4-tetrahydrothieno[2,3-d]pyrimidin-6-yl]methyl}hydrazinecarboxylate

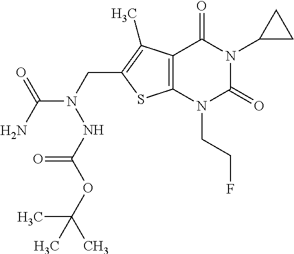 [SMILES: Cc1c(CN(NC(=O)OC(C)(C)C)C(N)=O)sc2c1c(=O)n(C1CC1)c(=O)n2CCF]

Analogously to the method described in Ex. 244A, 88 mg (0.337 mmol) of the compound from Ex. 418A and 57 μl (0.427 mmol) of trimethylsilyl isocyanate were used to prepare 71 mg (64% of theory, 88% purity) of the title compound. The reaction time here was about 16 h.

¹H-NMR (400 MHz, DMSO-d₆, δ/ppm): 8.91 (br. s, 1H), 6.17 (s, 2H), 4.71 (dt, 2H), 4.56 (broad, 2H), 4.16 (dt, 2H), 2.62 (tt, 1H), 2.32 (s, 3H), 1.38 (br. s, 9H), 1.08-0.96 (m, 2H), 0.73-0.60 (m, 2H).

LC/MS (Method 1, ESIneg): Rₜ=1.35 min, m/z=454.16 [M−H]⁻.

### Example 438A

### tert-Butyl 2-carbamoyl-2-{[3-cyclopropyl-1-(3-fluoropropyl)-5-methyl-2,4-dioxo-1,2,3,4-tetrahydrothieno[2,3-d]pyrimidin-6-yl]methyl}hydrazinecarboxylate

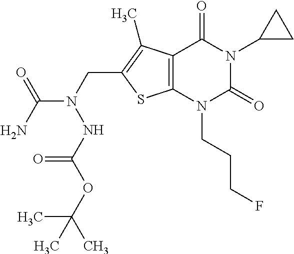 [SMILES: Cc1c(CN(NC(=O)OC(C)(C)C)C(N)=O)sc2c1c(=O)n(C1CC1)c(=O)n2CCCF]

Analogously to the method described in Ex. 244A, 176 mg (0.351 mmol, 85% purity) of the compound from Ex. 419A and 94 μl (0.702 mmol) of trimethylsilyl isocyanate were used to prepare 144 mg (87% of theory) of the title compound. A difference here from the process described above was that a further 47 μl (0.351 mmol) of trimethylsilyl isocyanate was added after 16 h and after 40 h of reaction time.

¹H-NMR (500 MHz, DMSO-d₆, δ/ppm): 8.90 (br. s, 1H), 6.17 (br. s, 2H), 4.70 (broad, 2H), 4.53 (dt, 2H), 3.95 (br. t, 2H), 2.64-2.56 (m, 1H), 2.32 (s, 3H), 2.11-1.99 (m, 2H), 1.38 (br. s, 9H), 1.06-0.96 (m, 2H), 0.71-0.58 (m, 2H).

LC/MS (Method 6, ESIneg): Rₜ=1.04 min, m/z=468 [M−H]⁻.

### Example 439A

### tert-Butyl 2-carbamoyl-2-{[3-cyclopropyl-1-(4-fluorobutyl)-5-methyl-2,4-dioxo-1,2,3,4-tetrahydrothieno[2,3-d]pyrimidin-6-yl]methyl}hydrazinecarboxylate

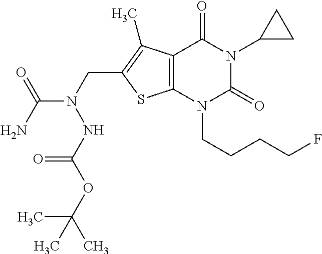 [SMILES: Cc1c(CN(NC(=O)OC(C)(C)C)C(N)=O)sc2c1c(=O)n(C1CC1)c(=O)n2CCCCF]

Analogously to the method described in Ex. 244A, 143 mg (0.292 mmol, 90% purity) of the compound from Ex. 420A and 78 μl (0.584 mmol) of trimethylsilyl isocyanate were used to prepare 135 mg (86% of theory, 91% purity) of the title compound. The reaction time here was about 16 h.

¹H-NMR (400 MHz, DMSO-d₆, δ/ppm): 8.90 (br. s, 1H), 6.18 (br. s, 2H), 4.56 (broad, 2H) 4.46 (dt, 2H), 3.87 (br. t, 2H), 2.61 (tt, 1H), 2.32 (s, 3H), 1.84-1.61 (m, 4H), 1.38 (br. s, 9H), 1.08-0.95 (m, 2H), 0.67-0.61 (m, 2H).

LC/MS (Method 1, ESIneg): Rₜ=1.50 min, m/z=482.19 [M−H].

### Example 440A

### tert-Butyl 2-carbamoyl-2-{[3-cyclopropyl-1-(2,2-difluoroethyl)-5-methyl-2,4-dioxo-1,2,3,4-tetrahydrothieno[2,3-d]pyrimidin-6-yl]methyl}hydrazinecarboxylate

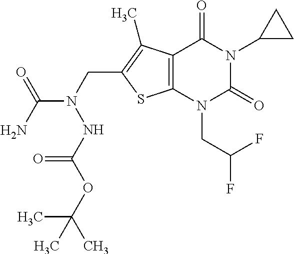 [SMILES: Cc1c(CN(NC(=O)OC(C)(C)C)C(N)=O)sc2c1c(=O)n(C1CC1)c(=O)n2CC(F)F]

Analogously to the method described in Ex. 244A, 130 mg (0.245 mmol, 81% purity) of the compound from Ex. 421A and 66 μl (0.489 mmol) of trimethylsilyl isocyanate were used to prepare 103 mg (80% of theory, 90% purity) of the title compound. The reaction time here was about 40 h.

¹H-NMR (400 MHz, DMSO-d₆, δ/ppm): 8.93 (br. s, 1H), 6.40 (dt, 1H), 6.18 (br. s, 2H), 4.56 (broad, 2H), 4.28 (td, 2H), 2.66-2.58 (m, 1H), 2.32 (s, 3H), 1.38 (br. s, 9H), 1.08-0.96 (m, 2H), 0.74-0.60 (m, 2H).

LC/MS (Method 1, ESIneg): Rₜ=1.42 min, m/z=472.15 [M−H].

### Example 441A

### tert-Butyl 2-carbamoyl-2-{[3-cyclopropyl-5-methyl-2,4-dioxo-1-(4,4,4-trifluorobutyl)-1,2,3,4-tetrahydrothieno[2,3-d]pyrimidin-6-yl]methyl}hydrazinecarboxylate

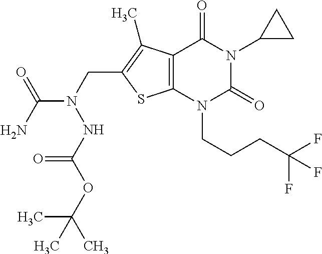 [SMILES: Cc1c(CN(NC(=O)OC(C)(C)C)C(N)=O)sc2c1c(=O)n(C1CC1)c(=O)n2CCCC(F)(F)F]

Analogously to the method described in Ex. 244A, 176 mg (0.314 mmol, 85% purity) of the compound from Ex. 422A and 84 μl (0.628 mmol) of trimethylsilyl isocyanate were used to prepare 159 mg (92% of theory, 95% purity) of the title compound. The reaction time here was about 16 h.

¹H-NMR (400 MHz, DMSO-d₆, δ/ppm): 8.89 (br. s, 1H), 6.18 (br. s, 2H), 4.57 (broad, 2H), 3.92 (br. t, 2H), 2.64-2.56 (m, 1H), 2.46-2.35 (m, 2H), 2.32 (s, 3H), 1.88 (quin, 2H), 1.38 (br. s, 9H), 1.07-0.94 (m, 2H), 0.72-0.59 (m, 2H).

LC/MS (Method 1, ESIneg): Rₜ=1.66 min, m/z=518.17 [M−H].

### Example 442A

### tert-Butyl 2-carbamoyl-2-({3-cyclopropyl-1-[(2,2-difluorocyclobutyl)methyl]-5-methyl-2,4-dioxo-1,2,3,4-tetrahydrothieno[2,3-d]pyrimidin-6-yl}methyl)hydrazinecarboxylate (enantiomer 1)

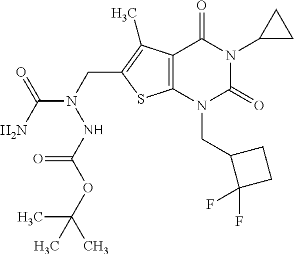 [SMILES: Cc1c(CN(NC(=O)OC(C)(C)C)C(N)=O)sc2c1c(=O)n(C1CC1)c(=O)n2CC1CCC1(F)F]

Analogously to the method described in Ex. 244A, 143 mg (0.304 mmol) of the compound from Ex. 423A and 82 μl (0.608 mmol) of trimethylsilyl isocyanate were used to prepare 143 mg (66% of theory, 73% purity) of the title compound. A difference here from the process described above was that the purification of the product by means of MPLC was dispensed with.

LC/MS (Method 1, ESIneg): Rₜ=1.62 min, m/z=512.18 [M−H]⁻.

### Example 443A

### tert-Butyl 2-carbamoyl-2-({3-cyclopropyl-1-[(2,2-difluorocyclobutyl)methyl]-5-methyl-2,4-dioxo-1,2,3,4-tetrahydrothieno[2,3-d]pyrimidin-6-yl}methyl)hydrazinecarboxylate (enantiomer 2)

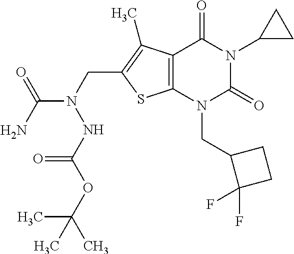 [SMILES: Cc1c(CN(NC(=O)OC(C)(C)C)C(N)=O)sc2c1c(=O)n(C1CC1)c(=O)n2CC1CCC1(F)F]

Analogously to the method described in Ex. 244A, 100 mg (0.213 mmol) of the compound from Ex. 424A and 57 μl (0.425 mmol) of trimethylsilyl isocyanate were used to prepare 128 mg (93% of theory, 80% purity) of the title compound. A difference here from the process described above was that the purification of the product by means of MPLC was dispensed with.

LC/MS (Method 1, ESIneg): Rₜ=1.62 min, m/z=512.18 [M−H].

### Example 444A

### tert-Butyl 2-carbamoyl-2-({3-cyclopropyl-1-[(3,3-difluorocyclobutyl)methyl]-5-methyl-2,4-dioxo-1,2,3,4-tetrahydrothieno[2,3-d]pyrimidin-6-yl}methyl)hydrazinecarboxylate

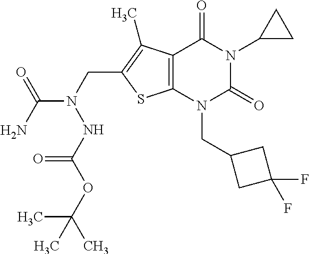 [SMILES: Cc1c(CN(NC(=O)OC(C)(C)C)C(N)=O)sc2c1c(=O)n(C1CC1)c(=O)n2CC1CC(F)(F)C1]

To a solution of 232 mg (0.468 mmol, 95% purity) of the compound from Ex. 425A in 15 ml of isopropanol were added 126 μl (0.937 mmol) of trimethylsilyl isocyanate, and the mixture was stirred at RT for 2.5 days. Since the conversion was still incomplete, a further 63 μl (468 mmol) of trimethylsilyl isocyanate were added and the stirring was continued at RT for 24 h. The reaction mixture was then concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and the concentrated. The residue was purified by means of MPLC (Biotage Isolera One, SNAP Ultra cartridge, 25 g of silica gel, eluent: cyclohexane/ethyl acetate 1:1). After concentration of the product fractions and drying under high vacuum, 232 mg (96% of theory) of the title compound were obtained.

¹H-NMR (400 MHz, DMSO-d₆, δ/ppm): 8.90 (br. s, 1H), 6.18 (br. s, 2H), 4.57 (broad, 2H), 4.09-3.96 (m, 2H), 2.74-2.56 (m, 4H), 2.32 (s, 3H), 1.38 (br. s, 9H), 1.08-0.96 (m, 2H), 0.69-0.57 (m, 2H).

LC/MS (Method 1, ESIneg): Rₜ=1.61 min, m/z=512.18 [M−H].

### Example 445A

### tert-Butyl 2-carbamoyl-2-({3-cyclopropyl-1-[(2,2-difluorocyclopentyl)methyl]-5-methyl-2,4-dioxo-1,2,3,4-tetrahydrothieno[2,3-d]pyrimidin-6-yl}methyl)hydrazinecarboxylate (enantiomer 1)

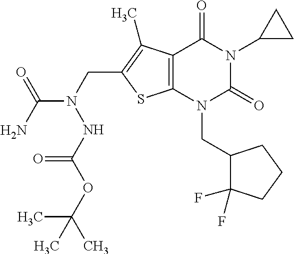 [SMILES: Cc1c(CN(NC(=O)OC(C)(C)C)C(N)=O)sc2c1c(=O)n(C1CC1)c(=O)n2CC1CCCC1(F)F]

Analogously to the method described in Ex. 244A, 120 mg (0.248 mmol) of the compound from Ex. 426A and 66 μl (0.495 mmol) of trimethylsilyl isocyanate were used to prepare 130 mg (80% of theory, 80% purity) of the title compound. The reaction time here was about 16 h.

LC/MS (Method 1, ESIneg): Rₜ=1.70 min, m/z=526.19 [M−H].

### Example 446A

### tert-Butyl 2-carbamoyl-2-({3-cyclopropyl-1-[(2,2-difluorocyclopentyl)methyl]-5-methyl-2,4-dioxo-1,2,3,4-tetrahydrothieno[2,3-d]pyrimidin-6-yl}methyl)hydrazinecarboxylate (enantiomer 2)

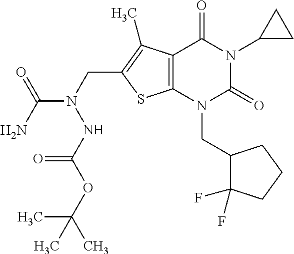 [SMILES: Cc1c(CN(NC(=O)OC(C)(C)C)C(N)=O)sc2c1c(=O)n(C1CC1)c(=O)n2CC1CCCC1(F)F]

To a solution of 127 mg (0.262 mmol) of the compound from Ex. 427A in 9 ml of isopropanol were added 70 μl (0.524 mmol) of trimethylsilyl isocyanate, and the mixture was stirred at RT for 16 h. The reaction mixture was then concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and concentrated. The residue was purified by means of MPLC (Biotage Isolera One, SNAP Ultra cartridge, 25 g of silica gel, eluent: cyclohexane/ethyl acetate 1:1). After concentration of the product fractions and drying under high vacuum, 138 mg (85% of theory, 85% purity) of the title compound were obtained.

LC/MS (Method 1, ESIneg): Rₜ=1.70 min, m/z=526.19 [M−H]⁻.

### Example 447A

### tert-Butyl 2-carbamoyl-2-({3-cyclopropyl-1-[(3,3-difluorocyclopentyl)methyl]-5-methyl-2,4-dioxo-1,2,3,4-tetrahydrothieno[2,3-d]pyrimidin-6-yl}methyl)hydrazinecarboxylate (racemate)

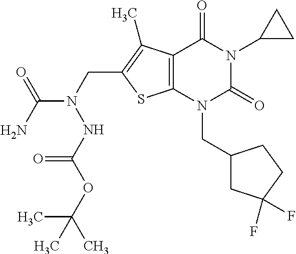 [SMILES: Cc1c(CN(NC(=O)OC(C)(C)C)C(N)=O)sc2c1c(=O)n(C1CC1)c(=O)n2CC1CCC(F)(F)C1]

Analogously to the method described in Ex. 244A, 238 mg (0.467 mmol, 95% purity) of the compound from Ex. 428A and 125 μl (0.933 mmol) of trimethylsilyl isocyanate were used to prepare 216 mg (78% of theory, 90% purity) of the title compound. The reaction time here was 6 days.

¹H-NMR (400 MHz, DMSO-d₆, δ/ppm): 8.90 (br. s, 1H), 6.18 (br. s, 2H), 4.57 (broad, 2H), 3.97-3.83 (m, 2H), 2.69-2.56 (m, 2H), 2.32 (s, 3H), 2.28-2.11 (m, 2H), 2.10-1.91 (m, 2H), 1.91-1.80 (m, 1H), 1.60 (dq, 1H), 1.38 (br. s, 9H), 1.08-0.93 (m, 2H), 0.67-0.60 (m, 2H).

LC/MS (Method 1, ESIneg): Rₜ=1.68 min, m/z=526.19 [M−H]⁻.

### Example 448A

### tert-Butyl 2-carbamoyl-2-{[1-(3-cyanopropyl)-3-cyclopropyl-5-methyl-2,4-dioxo-1,2,3,4-tetrahydrothieno[2,3-d]pyrimidin-6-yl]methyl}hydrazinecarboxylate

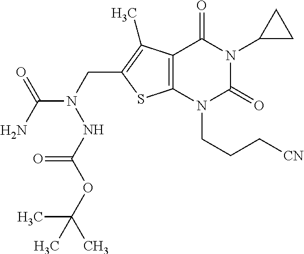 [SMILES: Cc1c(CN(NC(=O)OC(C)(C)C)C(N)=O)sc2c1c(=O)n(C1CC1)c(=O)n2CCCC#N]

Analogously to the method described in Ex. 244A, 125 mg (0.288 mmol) of the compound from Ex. 429A and 77 μl (0.577 mmol) of trimethylsilyl isocyanate were used to prepare 140 mg (96% of theory, 95% purity) of the title compound. The reaction time here was about 16 h.

¹H-NMR (400 MHz, DMSO-d₆, δ/ppm): 8.91 (br. s, 1H), 6.18 (br. s, 2H), 4.57 (broad, 2H), 3.94 (br. t, 2H), 2.65-2.56 (m, 3H), 2.32 (s, 3H), 2.02-1.90 (m, 2H), 1.38 (br. s, 9H), 1.06-0.95 (m, 2H), 0.73-0.59 (m, 2H).

LC/MS (Method 1, ESIneg): Rₜ=1.30 min, m/z=475.18 [M−H].

### Example 449A

### tert-Butyl 2-carbamoyl-2-[(3-cyclopropyl-5-methyl-2,4-dioxo-1-{2-[(trifluoromethyl)sulfanyl]ethyl}-1,2,3,4-tetrahydrothieno[2,3-d]pyrimidin-6-yl)methyl]hydrazinecarboxylate

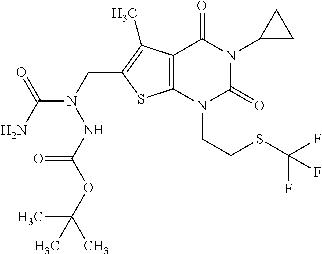 [SMILES: Cc1c(CN(NC(=O)OC(C)(C)C)C(N)=O)sc2c1c(=O)n(C1CC1)c(=O)n2CCSC(F)(F)F]

Analogously to the method described in Ex. 244A, 181 mg (0.337 mmol, 92% purity) of the compound from Ex. 430A and 90 μl (0.859 mmol) of trimethylsilyl isocyanate were used to prepare 154 mg (63% of theory, 75% purity) of the title compound. The reaction time here was about 40 h.

LC/MS (Method 1, ESIneg): Rₜ=1.70 min, m/z=536.12 [M−H].

### Example 450A

### tert-Butyl 2-carbamoyl-2-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)hydrazinecarboxylate (trans racemate)

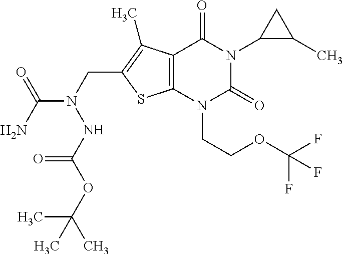 [SMILES: Cc1c(CN(NC(=O)OC(C)(C)C)C(N)=O)sc2c1c(=O)n(C1CC1C)c(=O)n2CCOC(F)(F)F]

To a solution of 324 mg (0.625 mmol, 95% purity) of the compound from Ex. 431A in 20 ml of isopropanol were added 168 μl (1.25 mmol) of trimethylsilyl isocyanate, and the mixture was stirred at RT for 16 h. Thereafter, the reaction mixture was concentrated to dryness and the residue that remained was purified by means of MPLC (Biotage Isolera One, SNAP Ultra cartridge, 25 g of silica gel, eluent: cyclohexane/ethyl acetate gradient). After concentration of the product fractions and drying under high vacuum, 278 mg (77% of theory, 92% purity) of the title compound were obtained.

¹H-NMR (400 MHz, DMSO-d₆, δ/ppm): 8.90 (br. s, 1H), 6.17 (br. s, 2H), 4.56 (broad, 2H), 4.38 (t, 2H), 4.23-4.09 (m, 2H), 2.32 (s, 3H), 2.27 (dt, 1H), 1.37 (br. s, 9H), 1.15 (d, 3H), 1.01-0.90 (m, 1H), 0.89-0.74 (m, 2H).

LC/MS (Method 1, ESIneg): Rₜ=1.75 min, m/z=534.16 [M−H].

### Example 451A

### tert-Butyl 2-carbamoyl-2-({1-(2-methoxyethyl)-5-methyl-2,4-dioxo-3-[2-(trifluoromethyl)cyclopropyl]-1,2,3,4-tetrahydrothieno[2,3-d]pyrimidin-6-yl}methyl)hydrazinecarboxylate (trans racemate)

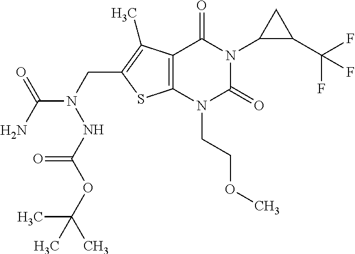 [SMILES: COCCn1c(=O)n(C2CC2C(F)(F)F)c(=O)c2c(C)c(CN(NC(=O)OC(C)(C)C)C(N)=O)sc21]

To a solution of 310 mg (0.617 mmol, 98% purity) of the compound from Ex. 432A in 17 ml of isopropanol were added 165 μl (1.23 mmol) of trimethylsilyl isocyanate, and the mixture was first stirred at RT for 40 h. Then the reaction mixture was stirred at 50° C. for 24 h and then left to stand at RT for 2 days. After adding a further 124 μl (0.926 mmol) of trimethylsilyl isocyanate, the reaction mixture was heated once again to 50° C. for 24 h. Then the reaction mixture was concentrated on a rotary evaporator to about half its original volume. In the course of this, the product precipitated out. The product was filtered off with suction, washed with diethyl ether and dried under high vacuum. 286 mg (81% of theory, 94% purity) of the title compound were obtained.

¹H-NMR (400 MHz, DMSO-d₆, δ/ppm): 8.94 (br. s, 1H), 6.18 (br. s, 2H), 4.56 (broad, 2H), 4.08-3.91 (m, 2H), 3.62 (t, 2H), 3.25 (s, 3H), 3.05-2.96 (m, 1H), 2.32 (s, 3H), 2.23 (dtd, 1H), 1.51 (q, 1H), 1.39 (br. s, 9H), 1.35-1.28 (m, 1H).

LC/MS (Method 2, ESIneg): Rₜ=0.91 min, m/z=534 [M−H].

### Example 452A

### tert-Butyl 2-carbamoyl-2-{[3-(2-ethylcyclopropyl)-1-(2-methoxyethyl)-5-methyl-2,4-dioxo-1,2,3,4-tetrahydrothieno[2,3-d]pyrimidin-6-yl]methyl}hydrazinecarboxylate (trans racemate)

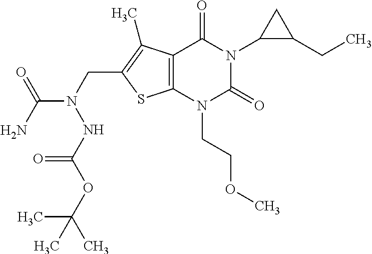 [SMILES: CCC1CC1n1c(=O)c2c(C)c(CN(NC(=O)OC(C)(C)C)C(N)=O)sc2n(CCOC)c1=O]

To a solution of 354 mg (0.767 mmol, 98% purity) of the compound from Ex. 433A in 16 ml of isopropanol were added 206 μl (1.53 mmol) of trimethylsilyl isocyanate, and the mixture was stirred at RT for about 18 h. The reaction mixture was then concentrated by about half. The product which precipitated out was filtered off with suction, washed with a little diethyl ether and dried under high vacuum. 340 mg (85% of theory, 95% purity) of the title compound were obtained.

¹H-NMR (400 MHz, DMSO-d₆, δ/ppm): 8.91 (br. s, 1H), 6.17 (br. s, 2H), 4.77-4.31 (broad, 2H), 4.08-3.88 (m, 2H), 3.61 (t, 2H), 3.24 (s, 3H), 2.39-2.24 (m, 1H), 2.31 (s, 3H), 1.63 (dquin, 1H), 1.38 (s, 9H), 1.28-1.17 (m, 1H), 1.03-0.94 (m, 1H), 0.99 (t, 3H), 0.84 (q, 1H), 0.80-0.70 (m, 1H).

LC/MS (Method 6, ESIneg): Rₜ=1.19 min, m/z=494 [M−H].

### Example 453A

### tert-Butyl 2-carbamoyl-2-{[3-(2-methoxycyclopropyl)-1-(2-methoxyethyl)-5-methyl-2,4-dioxo-1,2,3,4-tetrahydrothieno[2,3-d]pyrimidin-6-yl]methyl}hydrazinecarboxylate (trans racemate)

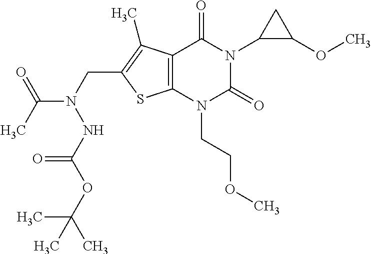 [SMILES: COCCn1c(=O)n(C2CC2OC)c(=O)c2c(C)c(CN(NC(=O)OC(C)(C)C)C(C)=O)sc21]

Analogously to the method described in Ex. 452A, 297 mg (0.653 mmol) of the compound from Ex. 434A and 175 μl (1.31 mmol) of trimethylsilyl isocyanate were used to obtain 275 mg (84% of theory) of the title compound.

¹H-NMR (500 MHz, DMSO-d₆, δ/ppm): 8.95 (br. s, 1H), 6.17 (br. s, 2H), 5.05-4.15 (m, 2H), 4.09-3.91 (m, 2H), 3.62 (t, 2H), 3.44-3.39 (m, 1H), 3.24 (s, 3H), 3.23 (s, 3H), 2.61 (dt, 1H), 2.31 (s, 3H), 1.39 (s, 9H), 1.31 (td, 1H), 0.92-0.85 (m, 1H).

LC/MS (Method 1, ESIneg): Rₜ=1.27 min, m/z=496.19 [M−H]⁻.

### Example 454A

### tert-Butyl 2-{[3-cyclopropyl-5-methyl-2,4-dioxo-1-(3,3,3-trifluoropropyl)-1,2,3,4-tetrahydrothieno[2,3-d]pyrimidin-6-yl]methyl}-2-[3-ethoxyprop-2-enoyl]hydrazinecarboxylate

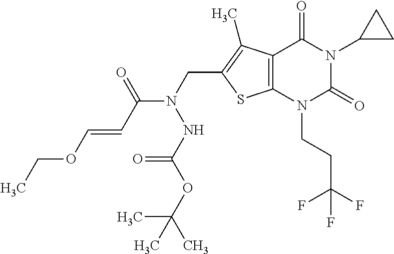 [SMILES: CCO/C=C/C(=O)N(Cc1sc2c(c1C)c(=O)n(C1CC1)c(=O)n2CCC(F)(F)F)NC(=O)OC(C)(C)C]

To a solution of 300 mg (0.649 mmol) of the compound from Ex. 202A in 7 ml of dichloromethane were added, at 0° C., 147 μl (0.843 mmol) of N,N-diisopropylethylamine and 123 mg (0.778 mmol, 85% purity) of 3-ethoxyacryloyl chloride. After the reaction mixture had been stirred at RT for about 16 h, it was diluted with further dichloromethane and washed with water. The organic phase was dried over anhydrous magnesium sulfate, filtered and concentrated. The remaining residue was purified by means of preparative HPLC (Method 11). The product fractions were combined and concentrated. After drying under high vacuum, 213 mg (52% of theory, 90% purity) of the title compound were obtained.

LC/MS (Method 1, ESIneg): Rₜ=1.99 min, m/z=559.18 [M−H]⁻.

### Example 455A

### tert-Butyl 2-[3-ethoxyprop-2-enoyl]-2-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methyl}hydrazinecarboxylate

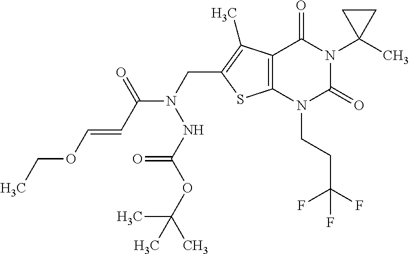 [SMILES: CCO/C=C/C(=O)N(Cc1sc2c(c1C)c(=O)n(C1(C)CC1)c(=O)n2CCC(F)(F)F)NC(=O)OC(C)(C)C]

Analogously to the method described in Ex. 454A, 300 mg (0.630 mmol) of the compound from Ex. 215A and 120 mg (0.755 mmol, 85% purity) of 3-ethoxyacryloyl chloride were used to obtain 315 mg (85% of theory, 98% purity) of the title compound.

LC/MS (Method 2, ESIneg): Rₜ=1.10 min, m/z=573 [M−H].

### Example 456A

### tert-Butyl 2-[3-ethoxyprop-2-enoyl]-2-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)hydrazinecarboxylate (trans racemate)

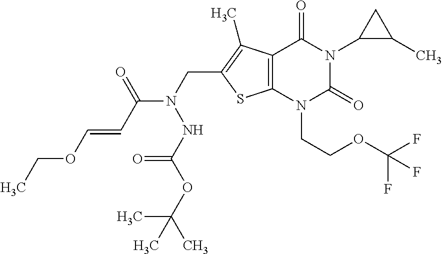 [SMILES: CCO/C=C/C(=O)N(Cc1sc2c(c1C)c(=O)n(C1CC1C)c(=O)n2CCOC(F)(F)F)NC(=O)OC(C)(C)C]

Analogously to the method described in Ex. 454A, 370 mg (0.751 mmol) of the compound from Ex. 431A and 143 mg (0.901 mmol, 85% purity) of 3-ethoxyacryloyl chloride were used to obtain 302 mg (65% of theory, 96% purity) of the title compound.

LC/MS (Method 2, ESIneg): Rₜ=1.13 min, m/z=589 [M−H].

### Example 457A

### tert-Butyl 2-{[3-cyclobutyl-5-methyl-2,4-dioxo-1-(3,3,3-trifluoropropyl)-1,2,3,4-tetrahydrothieno[2,3-d]pyrimidin-6-yl]methyl}-2-[3-ethoxyprop-2-enoyl]hydrazinecarboxylate

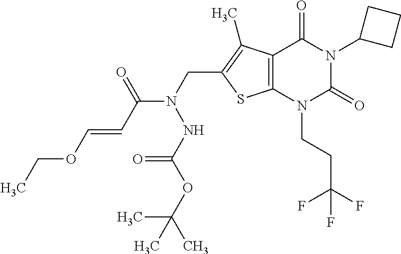 [SMILES: CCO/C=C/C(=O)N(Cc1sc2c(c1C)c(=O)n(C1CCC1)c(=O)n2CCC(F)(F)F)NC(=O)OC(C)(C)C]

Analogously to the method described in Ex. 454A, 250 mg (0.525 mmol) of the compound from Ex. 228A and 100 mg (0.630 mmol, 85% purity) of 3-ethoxyacryloyl chloride were used to obtain 225 mg (37% of theory, 50% purity) of the title compound.

LC/MS (Method 1, ESIneg): Rₜ=2.31 min, m/z=573.20 [M−H].

### Example 458A

### tert-Butyl 2-{[3-(3,3-difluorocyclobutyl)-5-methyl-2,4-dioxo-1-(3,3,3-trifluoropropyl)-1,2,3,4-tetrahydrothieno[2,3-d]pyrimidin-6-yl]methyl}-2-[3-ethoxyprop-2-enoyl]hydrazinecarboxylate

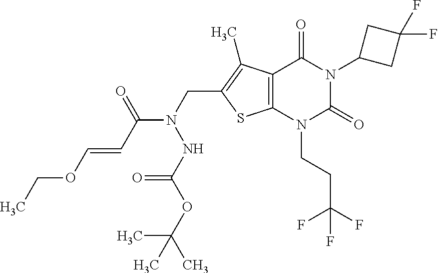 [SMILES: CCO/C=C/C(=O)N(Cc1sc2c(c1C)c(=O)n(C1CC(F)(F)C1)c(=O)n2CCC(F)(F)F)NC(=O)OC(C)(C)C]

Analogously to the method described in Ex. 454A, 300 mg (0.585 mmol) of the compound from Ex. 230A and 111 mg (0.702 mmol, 85% purity) of 3-ethoxyacryloyl chloride were used to obtain 320 mg (89% of theory) of the title compound.

LC/MS (Method 2, ESIneg): Rₜ=1.16 min, m/z=609 [M−H].

### Example 459A

### tert-Butyl 2-{[3-(3,3-dimethylcyclobutyl)-5-methyl-2,4-dioxo-1-(3,3,3-trifluoropropyl)-1,2,3,4-tetrahydrothieno[2,3-d]pyrimidin-6-yl]methyl}-2-[3-ethoxyprop-2-enoyl]hydrazinecarboxylate

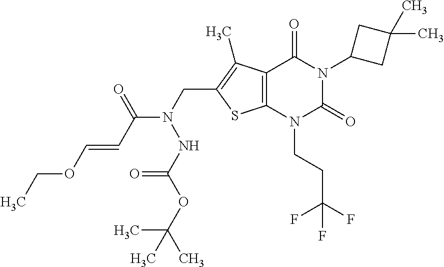 [SMILES: CCO/C=C/C(=O)N(Cc1sc2c(c1C)c(=O)n(C1CC(C)(C)C1)c(=O)n2CCC(F)(F)F)NC(=O)OC(C)(C)C]

Analogously to the method described in Ex. 454A, 280 mg (0.555 mmol) of the compound from Ex. 238A and 105 mg (0.666 mmol, 85% purity) of 3-ethoxyacryloyl chloride were used to obtain 275 mg (82% of theory) of the title compound.

LC/MS (Method 1, ESIneg): Rₜ=2.50 min, m/z=601.23 [M−H].

### Example 460A

### Diethyl 5-amino-3-ethylthiophene-2,4-dicarboxylate

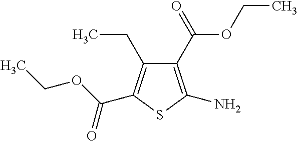 [SMILES: CCOC(=O)c1sc(N)c(C(=O)OCC)c1CC]

5.0 g (34.7 mmol) of ethyl 3-oxopentanoate and 3.92 g (34.7 mmol) of ethyl cyanoacetate were dissolved in 8 ml of ethanol, and 1.22 g (38.1 mmol) of sulfur were added. The mixture was heated to 45° C. and, at this temperature, 4.2 ml (39.9 mmol) of diethylamine were added dropwise. After the addition had ended, the reaction mixture was stirred at 60° C. for 16 h. It was then concentrated to dryness on a rotary evaporator. The remaining residue was taken up in 2.5 litres of water/ethyl acetate (1:1). After phase separation, the aqueous phase was extracted twice more with 200 ml each time of ethyl acetate. The combined organic extracts were washed with saturated aqueous sodium chloride solution, dried over anhydrous magnesium sulfate, filtered and concentrated. The crude product thus obtained was roughly prepurified by means of suction filtration through 300 g of silica gel with cyclohexane/ethyl acetate 10:1-5:1 as eluent to give the desired title compound and the isomeric ethyl 2-amino-4-(2-ethoxy-2-oxoethyl)-5-methylthiophene-3-carboxylate product. The title compound was isolated in sufficiently pure form from the fraction comprising the title compound by means of MPLC (Biotage Isolera, SNAP Ultra cartridge, 100 g of silica gel, cyclohexane/ethyl acetate 5:1). Yield: 1.80 g (18% of theory, 96% purity).

¹H-NMR (400 MHz, DMSO-d₆, δ/ppm): 7.93 (s, 2H), 4.23 (q, 2H), 4.17 (q, 2H), 3.17 (q, 2H), 1.28 (t, 3H), 1.24 (t, 3H), 1.07 (t, 3H).

LC/MS (Method 1, ESIpos): Rₜ=2.04 min, m/z=272.10 [M+H]⁺.

### Example 461A

### Diethyl 3-ethyl-5-{[(1-methylcyclopropyl)carbamoyl]amino}thiophene-2,4-dicarboxylate

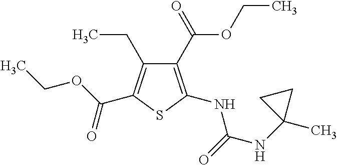 [SMILES: CCOC(=O)c1sc(NC(=O)NC2(C)CC2)c(C(=O)OCC)c1CC]

Analogously to the process described in Ex. 2A, 1.75 g (6.45 mmol) of the compound from Ex. 460A, 2.09 g (12.9 mmol) of 1,1′-carbonyldiimidazole (CDI) and 1.46 g (12.9 mmol) of 1-methylcyclopropanamine hydrochloride were used to obtain 2.09 g (87% of theory) of the title compound. The reaction time after addition of the 1-methylcyclopropanamine hydrochloride here was 5 h, and there was a final purification of the product by means of MPLC (Biotage Isolera, SNAP KP-Sil cartridge, 100 g of silica gel, cyclohexane/ethyl acetate 5:1).

¹H-NMR (400 MHz, DMSO-d₆, δ/ppm): 10.47 (br. s, 1H), 8.34 (br. s, 1H), 4.33 (q, 2H), 4.23 (q, 2H), 3.22 (q, 2H), 1.33 (s, 3H and t, 3H), 1.28 (t, 3H), 1.10 (t, 3H), 0.91-0.38 (m, 4H).

LC/MS (Method 2, ESIpos): Rₜ=1.18 min, m/z=369 [M+H]⁺.

### Example 462A

### Ethyl 5-ethyl-3-(1-methylcyclopropyl)-2,4-dioxo-1,2,3,4-tetrahydrothieno[2,3-d]pyrimidine-6-carboxylate

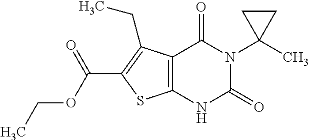 [SMILES: CCOC(=O)c1sc2[nH]c(=O)n(C3(C)CC3)c(=O)c2c1CC]

Analogously to the process described in Ex. 18A, 2.08 g (5.64 mmol) of the compound from Ex. 461A were used to prepare 1.68 g (92% of theory) of the title compound.

¹H-NMR (400 MHz, DMSO-d₆, δ/ppm): 12.26 (s, 1H), 4.26 (q, 2H), 3.28 (q, 2H), 1.33 (s, 3H), 1.28 (t, 3H), 1.11 (t, 3H), 0.96-0.78 (m, 4H).

LC/MS (Method 1, ESIpos): Rₜ=1.83 min, m/z=323.11 [M+H]⁺.

### Example 463A

### Ethyl 5-ethyl-3-(1-methylcyclopropyl)-2,4-dioxo-1-(3,3,3-trifluoropropyl)-1,2,3,4-tetrahydrothieno[2,3-d]pyrimidine-6-carboxylate

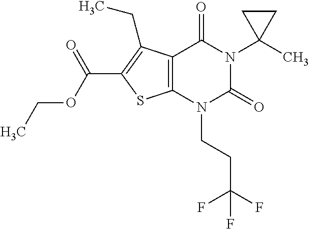 [SMILES: CCOC(=O)c1sc2c(c1CC)c(=O)n(C1(C)CC1)c(=O)n2CCC(F)(F)F]

Analogously to the process described in Ex. 45A, 870 mg (2.70 mmol) of the compound from Ex. 462A and 1.81 g (8.10 mmol) of 1,1,1-trifluoro-3-iodopropane were used to prepare 1.10 g (87% of theory, 90% purity) of the title compound. The reaction here was not effected at RT, but at 50° C.

¹H-NMR (400 MHz, DMSO-d₆, δ/ppm): 4.29 (q, 2H), 4.23-3.96 (m, 2H), 3.38-3.23 (m, 2H, partially concealed by water signal), 2.86-2.70 (m, 2H), 1.35 (s, 3H), 1.30 (t, 3H), 1.14 (t, 3H), 0.99-0.77 (m, 4H).

LC/MS (Method 2, ESIpos): Rₜ=1.22 min, m/z=419 [M+H]⁺.

### Example 464A

### Ethyl 5-ethyl-1-(2-methoxyethyl)-3-(1-methylcyclopropyl)-2,4-dioxo-1,2,3,4-tetrahydrothieno[2,3-d]pyrimidine-6-carboxylate

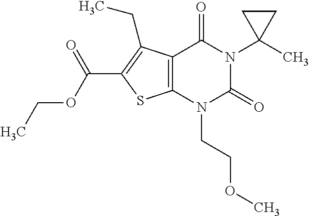 [SMILES: CCOC(=O)c1sc2c(c1CC)c(=O)n(C1(C)CC1)c(=O)n2CCOC]

Analogously to the method described in Ex. 45A, 870 mg (2.70 mmol) of the compound from Ex. 462A and 750 mg (5.40 mmol) of 2-bromoethyl methyl ether were used to prepare 975 mg (94% of theory) of the title compound. The reaction here was not effected at RT, but at 50° C.

¹H-NMR (400 MHz, DMSO-d₆, δ/ppm): 4.28 (q, 2H), 4.20-3.84 (m, 2H), 3.64 (br. t, 2H), 3.25 (s, 3H), 1.35 (s, 3H), 1.29 (t, 3H), 1.13 (t, 3H), 1.00-0.75 (m, 4H).

LC/MS (Method 2, ESIpos): Rₜ=1.12 min, m/z=381 [M+H]⁺.

### Example 465A

### 5-Ethyl-6-(hydroxymethyl)-3-(1-methylcyclopropyl)-1-(3,3,3-trifluoropropyl)thieno[2,3-d]pyrimidine-2,4(1H,3H)-dione

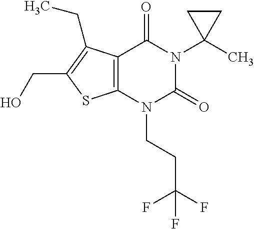 [SMILES: CCc1c(CO)sc2c1c(=O)n(C1(C)CC1)c(=O)n2CCC(F)(F)F]

1.04 g (2.49 mmol) of the compound from Ex. 463A were dissolved in 25 ml of anhydrous THF, and 2.5 ml (2.49 mmol) of a 1 M solution of lithium aluminium hydride in THF were added dropwise at −78° C. After 60 min, the reaction mixture was warmed to about −20° C. and the stirring was continued at this temperature. After 1 h, saturated aqueous ammonium chloride solution was added cautiously and the mixture was then extracted with ethyl acetate. The organic extract was dried over anhydrous magnesium sulfate, filtered and concentrated. The solids that remained were purified by means of MPLC (Biotage Isolera, SNAP Ultra cartridge, 50 g of silica gel, cyclohexane/ethyl acetate 2:1). After concentration of the product fractions and drying under high vacuum, 875 mg (84% of theory, 90% purity) of the title compound were obtained.

¹H-NMR (400 MHz, DMSO-d₆, δ/ppm): 5.61 (t, 1H), 4.59 (d, 2H), 4.21-3.94 (m, 2H), 2.87-2.65 (m, 4H), 1.34 (s, 3H), 1.07 (t, 3H), 0.97-0.75 (m, 4H).

LC/MS (Method 2, ESIpos): Rₜ=0.91 min, m/z=377 [M+H]⁺.

### Example 466A

### 5-Ethyl-6-(hydroxymethyl)-1-(2-methoxyethyl)-3-(1-methylcyclopropyl)thieno[2,3-d]pyrimidine-2,4(1H,3H)-dione

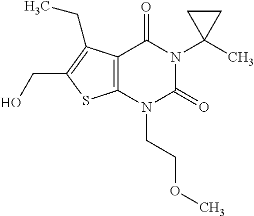 [SMILES: CCc1c(CO)sc2c1c(=O)n(C1(C)CC1)c(=O)n2CCOC]

Analogously to the method described in Ex. 465A, 965 mg (2.54 mmol) of the compound from Ex. 464A and 2.5 ml (2.54 mmol) of a 1 M solution of lithium aluminium hydride in THF were used to obtain 713 mg (83% of theory) of the title compound.

¹H-NMR (400 MHz, DMSO-d₆, δ/ppm): 5.55 (t, 1H), 4.57 (d, 2H), 4.16-3.83 (m, 2H), 3.62 (t, 2H), 3.25 (s, 3H), 2.88-2.68 (m, 2H), 1.34 (s, 3H), 1.07 (t, 3H), 0.97-0.75 (m, 4H).

LC/MS (Method 1, ESIpos): Rₜ=1.49 min, m/z=339.14 [M+H]⁺.

### Example 467A

### 5-Ethyl-3-(1-methylcyclopropyl)-2,4-dioxo-1-(3,3,3-trifluoropropyl)-1,2,3,4-tetrahydrothieno[2,3-d]pyrimidine-6-carbaldehyde

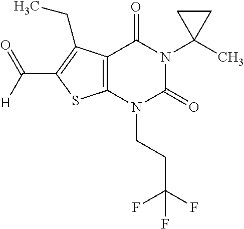 [SMILES: CCc1c(C=O)sc2c1c(=O)n(C1(C)CC1)c(=O)n2CCC(F)(F)F]

702 mg (1.86 mmol) of the compound from Example 465A were dissolved in 20 ml of anhydrous DMSO, and a little 4 Å molecular sieve and 2.6 mg (18.6 mmol) of triethylamine were added. Then, at intervals of 10 min, three portions each of 297 mg (1.86 mmol) of sulfur trioxide-pyridine complex were added. After 1.5 h and after a further 20 h, 297 mg (1.86 mmol) of sulfur trioxide-pyrimidine complex were added once again. After a further 30 min, the reaction mixture was diluted with ethyl acetate and washed successively with water and saturated aqueous sodium chloride solution. The mixture was dried over anhydrous magnesium sulfate, filtered and concentrated. The residue that remained was taken up with a little toluene three times and concentrated again each time. Then the solids were purified by means of MPLC (Biotage Isolera, SNAP Ultra cartridge, 25 g of silica gel, cyclohexane/ethyl acetate 2:1). After concentration of the product fractions and drying under high vacuum, 121 mg (11% of theory, 66% purity) of the title compound were obtained.

LC/MS (Method 1, ESIpos): Rₜ=2.01 min, m/z=375.10 [M+H]⁺.

### Example 468A

### 5-Ethyl-1-(2-methoxyethyl)-3-(1-methylcyclopropyl)-2,4-dioxo-1,2,3,4-tetrahydrothieno[2,3-d]pyrimidine-6-carbaldehyde

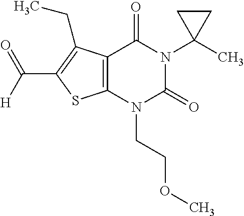 [SMILES: CCc1c(C=O)sc2c1c(=O)n(C1(C)CC1)c(=O)n2CCOC]

At −60° C., a solution of 142 μl (2.00 mmol) of anhydrous DMSO in 1 ml of dichloromethane was added dropwise to a solution of 128 μl (1.46 mmol) of oxalyl chloride in 1 ml of dichloromethane.

Subsequently, at the same temperature and over a period of 30 min, a solution of 450 mg (1.33 mmol) of the compound from Ex. 466A in 3.5 ml of dichloromethane was added. The reaction mixture was subsequently stirred at −60° C. for 1.5 h. Then, at the same temperature, a solution of 927 μl (6.65 mmol) of triethylamine in 1 ml of dichloromethane was added. The reaction mixture was warmed to 0° C. and stirred at this temperature for another 20 min. Then, at RT, the mixture was diluted with 50 ml of dichloromethane and washed successively with water and saturated aqueous sodium chloride solution. The organic phase was dried over anhydrous magnesium sulfate, filtered and concentrated. The solids that remained were purified by means of MPLC (Biotage Isolera, SNAP Ultra cartridge, 25 g of silica gel, cyclohexane/ethyl acetate 2:1). After concentration of the product fractions and drying under high vacuum, 57 mg (10% of theory, 84% purity) of the title compound were obtained.

LC/MS (Method 1, ESIpos): Rₜ=1.77 min, m/z=337.12 [M+H]⁺.

### Example 469A

### 6-{[(2-Aminoethyl)amino]methyl}-5-ethyl-3-(1-methylcyclopropyl)-1-(3,3,3-trifluoropropyl)thieno[2,3-d]pyrimidine-2,4(1H,3H)-dione

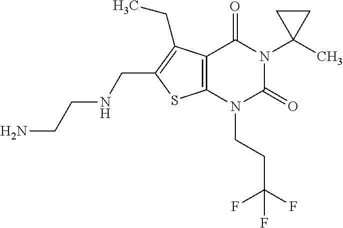 [SMILES: CCc1c(CNCCN)sc2c1c(=O)n(C1(C)CC1)c(=O)n2CCC(F)(F)F]

Analogously to the method described in Ex. 103A, 120 mg (0.321 mmol) of the compound from Ex. 467A, 129 μl (1.92 mmol) of 1,2-diaminoethane and 81 mg (1.28 mmol) of sodium cyanoborohydride were used to prepare 160 mg (78% of theory, 66% purity) of the title compound.

LC/MS (Method 1, ESIpos): Rₜ=0.88 min, m/z=419.17 [M+H]⁺.

### Example 470A

### 6-{[(2-Aminoethyl)amino]methyl}-5-ethyl-1-(2-methoxyethyl)-3-(1-methylcyclopropyl)thieno[2,3-d]pyrimidine-2,4(1H,3H)-dione

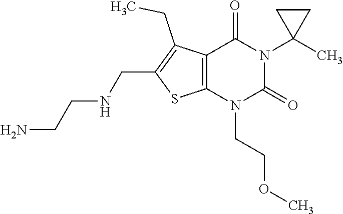 [SMILES: CCc1c(CNCCN)sc2c1c(=O)n(C1(C)CC1)c(=O)n2CCOC]

Analogously to the method described in Ex. 103A, 55 mg (0.166 mmol) of the compound from Ex. 468A, 67 μl (0.997 mmol) of 1,2-diaminoethane and 42 mg (0.665 mmol) of sodium cyanoborohydride were used to prepare 59 mg (61% of theory, 66% purity) of the title compound. A difference here from the process described above was that, after 16 h of reaction time, the same amounts again of 1,2-diaminoethane, acetic acid and sodium cyanoborohydride were added, and the reaction mixture was stirred at 60° C. for a further 20 h.

LC/MS (Method 1, ESIpos): Rₜ=0.68 min, m/z=321.13 [M+H−C₂H₈N₂]⁺.

### Example 471A

### tert-Butyl 2-({1,5-dimethyl-3-[(1S,2S)-2-methylcyclopropyl]-2,4-dioxo-1,2,3,4-tetrahydrothieno[2,3-d]pyrimidin-6-yl}methylene)hydrazinecarboxylate

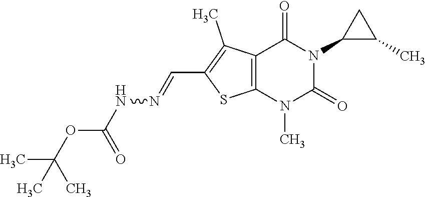 [SMILES: Cc1c(C=NNC(=O)OC(C)(C)C)sc2c1c(=O)n([C@H]1C[C@@H]1C)c(=O)n2C]

To a solution of 313 mg (1.13 mmol) of the compound from Ex. 362A in 11 ml of ethanol were added 223 mg (1.69 mmol) of tert-butyl hydrazinecarboxylate and 3 drops of conc. hydrochloric acid. After the reaction mixture had been stirred at RT for about 16 h, 150 ml of cold water were added and the mixture was neutralized by adding saturated sodium hydrogencarbonate solution. In the course of this, the product precipitated out. The product was filtered off with suction, washed with a little water and dried under high vacuum. 399 mg (90% of theory) of the title compound were obtained.

¹H-NMR (500 MHz, DMSO-d₆, δ/ppm): 10.83 (br. s, 1H), 8.28 (br. s, 1H), 3.41 (s, 3H), 2.44 (s, 3H), 2.27-2.19 (m, 1H), 1.45 (s, 9H), 1.15 (d, 3H), 1.04-0.94 (m, 1H), 0.88-0.78 (m, 2H).

LC/MS (Method 2, ESIneg): Rₜ=1.00 min, m/z=391 [M−H].

### Example 472A

### tert-Butyl 2-({1,5-dimethyl-3-[(1S,2S)-2-methylcyclopropyl]-2,4-dioxo-1,2,3,4-tetrahydrothieno[2,3-d]pyrimidin-6-yl}methyl)hydrazinecarboxylate

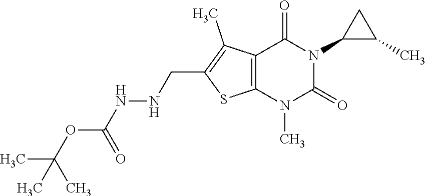 [SMILES: Cc1c(CNNC(=O)OC(C)(C)C)sc2c1c(=O)n([C@H]1C[C@@H]1C)c(=O)n2C]

To a solution of 399 mg (1.02 mmol) of the compound from Ex. 471A in 13 ml of methanol were added 326 mg (5.08 mmol) of sodium cyanoborohydride and a little Bromocresol Green. Subsequently, a sufficient amount of acetic acid was added by titration that the indicator colour just changed from blue to yellow. Then the reaction mixture was heated to 65° C. Over the entire reaction time, by addition of further acetic acid, the pH was constantly regulated such that the indicator colour just remained yellow. After a reaction time of 4 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 10 g of silica gel, eluent: cyclohexane/ethyl acetate 1:1). The product fractions were combined and concentrated. Drying of the residue under high vacuum gave 329 mg (75% of theory, 92% purity) of the title compound.

¹H-NMR (500 MHz, DMSO-d₆, δ/ppm): 8.23 (br. s, 1H), 5.05-4.88 (br. m, 1H), 3.96 (br. d, 2H), 3.38 (s, 3H), 2.31 (s, 3H), 2.23 (td, 1H), 1.38 (s, 9H), 1.15 (d, 3H), 1.02-0.92 (m, 1H), 0.82 (dd, 2H).

LC/MS (Method 1, ESIpos): Rₜ=1.71 min, m/z=263.08 [M+H−C₅H₁₂N₂O₂]⁺.

### Example 473A

### tert-Butyl 2-carbamoyl-2-({1,5-dimethyl-3-[(1S,2S)-2-methylcyclopropyl]-2,4-dioxo-1,2,3,4-tetrahydrothieno[2,3-d]pyrimidin-6-yl}methyl)hydrazinecarboxylate

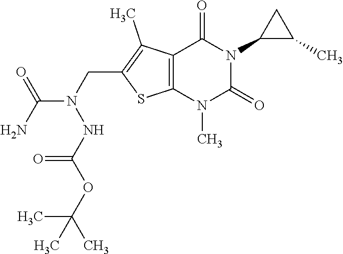 [SMILES: Cc1c(CN(NC(=O)OC(C)(C)C)C(N)=O)sc2c1c(=O)n([C@H]1C[C@@H]1C)c(=O)n2C]

To a solution of 329 mg (0.767 mmol, 92% purity) of the compound from Ex. 472A in 15 ml of isopropanol were added 206 μl (1.54 mmol) of trimethylsilyl isocyanate, and the mixture was stirred at RT for about 18 h. Thereafter, the reaction mixture was concentrated to about half the original volume. In the course of this, the product precipitated out. It was filtered off with suction and washed with a little diethyl ether. After drying under high vacuum, 273 mg (66% of theory, 82% purity) of the title compound were obtained.

LC/MS (Method 1, ESIpos): Rₜ=1.46 min, m/z=438.18 [M+H]⁺.

### Example 474A

### 1-Ethyl-5-methyl-3-[(1S,2S)-2-methylcyclopropyl]-2,4-dioxo-1,2,3,4-tetrahydrothieno[2,3-d]pyrimidine-6-carbaldehyde

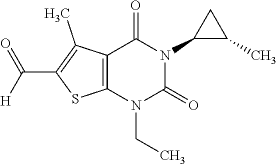 [SMILES: CCn1c(=O)n([C@H]2C[C@@H]2C)c(=O)c2c(C)c(C=O)sc21]

Analogously to the method described in Ex. 362A, 300 mg (1.14 mmol) of the compound from Ex. 303A and 272 μl (3.41 mmol) of ethyl iodide were used to prepare 346 mg (99% of theory, 95% purity) of the title compound.

¹H-NMR (500 MHz, DMSO-d₆, δ/ppm): 10.08 (s, 1H), 3.98-3.88 (m, 2H), 2.77 (s, 3H), 2.29-2.23 (m, 1H), 1.24 (t, 3H), 1.15 (d, 3H), 1.07-0.97 (m, 1H), 0.90-0.80 (m, 2H).

LC/MS (Method 1, ESIpos): Rₜ=1.69 min, m/z=293.09 [M+H]⁺.

### Example 475A

### 5-Methyl-3-[(1S,2S)-2-methylcyclopropyl]-2,4-dioxo-1-propyl-1,2,3,4-tetrahydrothieno[2,3-d]pyrimidine-6-carbaldehyde

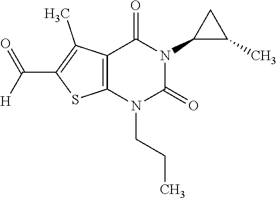 [SMILES: CCCn1c(=O)n([C@H]2C[C@@H]2C)c(=O)c2c(C)c(C=O)sc21]

Analogously to the process described in Ex. 362A, 300 mg (1.14 mmol) of the compound from Ex. 303A and 332 μl (3.41 mmol) of propyl iodide were used to prepare 321 mg (92% of theory) of the title compound. The reaction was effected here at 100° C. in a microwave oven (Biotage Initiator with dynamic control of irradiation power).

¹H-NMR (500 MHz, DMSO-d₆, δ/ppm): 10.07 (s, 1H), 3.91-3.79 (m, 2H), 2.77 (s, 3H), 2.29-2.23 (m, 1H), 1.70 (sext, 2H), 1.15 (d, 3H), 1.07-0.96 (m, 1H), 0.92 (t, 3H), 0.88-0.81 (m, 2H).

LC/MS (Method 1, ESIpos): Rₜ=1.87 min, m/z=307.11 [M+H]⁺.

### Example 476A

### 1-(2-Fluoroethyl)-5-methyl-3-[(1S,2S)-2-methylcyclopropyl]thieno[2,3-d]pyrimidine-2,4(1H,3H)-dione

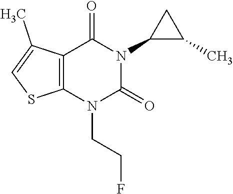 [SMILES: Cc1csc2c1c(=O)n([C@H]1C[C@@H]1C)c(=O)n2CCF]

300 mg (1.27 mmol) of the compound from Ex. 302A were dissolved in 5 ml of anhydrous DMF, 620 mg (1.90 mmol) of caesium carbonate were added, and the mixture was stirred at RT for 15 min. Then 316 μl (3.81 mmol) of 1-fluoro-2-iodoethane were added. After the reaction mixture had been stirred in a microwave oven (Biotage Initiator with dynamic control of irradiation power) at 100° C. for 3 h, it was poured onto water. In the course of this, the product precipitated out. After stirring at RT for 1 h, the solids were filtered off with suction, washed with a little water and dried under high vacuum. 326 mg (90% of theory) of the title compound were obtained.

¹H-NMR (500 MHz, DMSO-d₆, δ/ppm): 6.78 (d, 1H), 4.73 (dt, 2H), 4.16 (dq, 2H), 2.36 (d, 3H), 2.25 (dt, 1H), 1.15 (d, 3H), 1.02 (tq, 1H), 0.90-0.79 (m, 2H).

LC/MS (Method 3, ESIpos): Rₜ=2.47 min, m/z=283 [M+H]⁺.

### Example 477A

### 1-(2-Fluoroethyl)-5-methyl-3-[(1S,2S)-2-methylcyclopropyl)-2,4-dioxo-1,2,3,4-tetrahydrothieno[2,3-d]pyrimidine-6-carbaldehyde

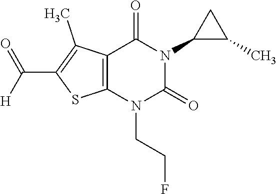 [SMILES: Cc1c(C=O)sc2c1c(=O)n([C@H]1C[C@@H]1C)c(=O)n2CCF]

To a solution of 320 mg (1.13 mmol) of the compound from Ex. 476A in 0.9 ml (11.3 mmol) of DMF were cautiously added 1.3 ml (13.6 mmol) of phosphorus oxychloride. After the strongly exothermic reaction had abated, the mixture was stirred for another 15 min. Then the reaction mixture was stirred cautiously into 30 ml of water. After stirring at RT for about 1 h, the precipitated product was filtered off with suction, washed to neutrality with water and dried under high vacuum. 322 mg (88% of theory, 96% purity) of the title compound were obtained.

¹H-NMR (500 MHz, DMSO-d₆, δ/ppm): 10.08 (s, 1H), 4.74 (dt, 2H), 4.23 (dm, 2H), 2.77 (s, 3H), 2.27 (dt, 1H), 1.15 (d, 3H), 1.09-0.99 (m, 1H), 0.92-0.80 (m, 2H).

LC/MS (Method 2, ESIpos): Rₜ=0.87 min, m/z=311 [M+H]⁺.

### Example 478A

### 1-(2,2-Difluoroethyl)-5-methyl-3-[(1S,2S)-2-methylcyclopropyl]thieno[2,3-d]pyrimidine-2,4(1H,3H)-dione

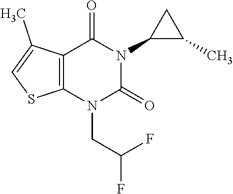 [SMILES: Cc1csc2c1c(=O)n([C@H]1C[C@@H]1C)c(=O)n2CC(F)F]

Analogously to the method described in Ex. 476A, 300 mg (1.27 mmol) of the compound from Ex. 302A and 348 μl (3.81 mmol) of 1,1-difluoro-2-iodoethane were used to prepare 346 mg (86% of theory, 95% purity) of the title compound.

¹H-NMR (500 MHz, DMSO-d₆, δ/ppm): 6.82 (d, 1H), 6.33 (tt, 1H), 4.37-4.20 (m, 2H), 2.37 (d, 3H), 2.26 (dt, 1H), 1.15 (d, 3H), 1.07-0.97 (m, 1H), 0.90-0.80 (m, 2H).

LC/MS (Method 1, ESIpos): Rₜ=1.81 min, m/z=301.08 [M+H]⁺.

### Example 479A

### 1-(2,2-Difluoroethyl)-5-methyl-3-[(1S,2S)-2-methylcyclopropyl)-2,4-dioxo-1,2,3,4-tetrahydrothieno[2,3-d]pyrimidine-6-carbaldehyde

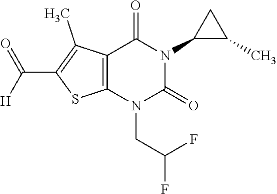 [SMILES: Cc1c(C=O)sc2c1c(=O)n([C@H]1C[C@@H]1C)c(=O)n2CC(F)F]

Analogously to the method described in Ex. 477A, 340 mg (1.08 mmol, 95% purity) of the compound from Ex. 478A, 0.9 ml (11.3 mmol) of DMF and 1.3 ml (13.6 mmol) of phosphorus oxychloride were used to prepare 335 mg (89% of theory, 94% purity) of the title compound.

¹H-NMR (500 MHz, DMSO-d₆, δ/ppm): 10.09 (s, 1H), 6.34 (tt, 1H), 4.47-4.29 (m, 2H), 2.77 (s, 3H), 2.28 (dt, 1H), 1.15 (d, 3H), 1.10-0.99 (m, 1H), 0.92-0.81 (m, 2H).

LC/MS (Method 2, ESIpos): Rₜ=0.92 min, m/z=329 [M+H]⁺.

### Example 480A

### 5-Methyl-3-[(1S,2S)-2-methylcyclopropyl]-1-(2,2,2-trifluoroethyl)thieno[2,3-d]pyrimidine-2,4(1H,3H)-dione

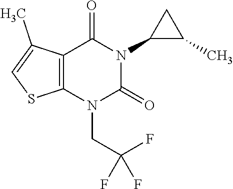 [SMILES: Cc1csc2c1c(=O)n([C@H]1C[C@@H]1C)c(=O)n2CC(F)(F)F]

300 mg (1.27 mmol) of the compound from Ex. 302A were dissolved in 5 ml of anhydrous DMF, 620 mg (1.90 mmol) of caesium carbonate were added, and the mixture was stirred at RT for 10 min. Then 375 μl (3.81 mmol) of 1,1,1-trifluoro-2-iodoethane were added. After the reaction mixture had been stirred in a microwave oven (Biotage Initiator with dynamic control of irradiation power) at 100° C. for 4 h, it was diluted with ethyl acetate and washed successively with water and saturated sodium chloride solution. After drying over anhydrous magnesium sulfate, the mixture was filtered and concentrated. The crude product thus obtained was purified by means of preparative HPLC (Method 11). The product fraction was concentrated by evaporation and the product was dried under high vacuum. 195 mg (48% of theory) of the title compound were obtained.

¹H-NMR (500 MHz, DMSO-d₆, δ/ppm): 6.86 (d, 1H), 4.88-4.71 (m, 2H), 2.38 (d, 3H), 2.32-2.27 (m, 1H), 1.16 (d, 3H), 1.07-0.97 (m, 1H), 0.89-0.82 (m, 2H).

LC/MS (Method 1, ESIpos): Rₜ=1.96 min, m/z=319.07 [M+H]⁺.

### Example 481A

### 5-Methyl-3-[(1S,2S)-2-methylcyclopropyl]-2,4-dioxo-1-(2,2,2-trifluoroethyl)-1,2,3,4-tetrahydrothieno[2,3-d]pyrimidine-6-carbaldehyde

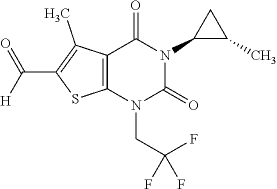 [SMILES: Cc1c(C=O)sc2c1c(=O)n([C@H]1C[C@@H]1C)c(=O)n2CC(F)(F)F]

Analogously to the method described in Ex. 477A, 190 mg (0.597 mmol,) of the compound from Ex. 480A, 0.5 ml (5.97 mmol) of DMF and 0.7 ml (7.16 mmol) of phosphorus oxychloride were used to prepare 183 mg (84% of theory, 95% purity) of the title compound.

¹H-NMR (500 MHz, DMSO-d₆, δ/ppm): 10.10 (s, 1H), 4.99-4.81 (m, 2H), 2.78 (s, 3H), 2.34-2.27 (m, 1H), 1.16 (d, 3H), 1.08-0.98 (m, 1H), 0.91-0.81 (m, 2H).

LC/MS (Method 2, ESIpos): Rₜ=1.00 min, m/z=347 [M+H]⁺.

### Example 482A

### 1-(3-Fluoropropyl)-5-methyl-3-[(1S,2S)-2-methylcyclopropyl]-2,4-dioxo-1,2,3,4-tetrahydrothieno[2,3-d]pyrimidine-6-carbaldehyde

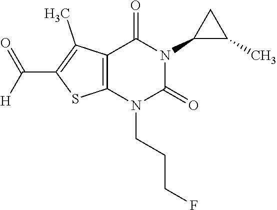 [SMILES: Cc1c(C=O)sc2c1c(=O)n([C@H]1C[C@@H]1C)c(=O)n2CCCF]

300 mg (1.14 mmol) of the compound from Ex. 303A were dissolved in 2.9 ml of anhydrous DMF, 555 mg (1.70 mmol) of caesium carbonate were added, and the mixture was stirred at RT for 10 min. Then 348 μl (3.41 mmol) of 1-fluoro-3-iodopropane were added. After the reaction mixture had been stirred in a microwave oven (Biotage Initiator with dynamic control of irradiation power) at 100° C. for 4 h, it was poured onto water and acidified by addition of 1 M hydrochloric acid. In the course of this, the product precipitated out. After stirring at RT for 1 h, the product was filtered off with suction, washed with a little water and dried under high vacuum. 364 mg (98% of theory) of the title compound were obtained.

¹H-NMR (500 MHz, DMSO-d₆, δ/ppm): 10.08 (s, 1H), 4.54 (dt, 2H), 4.08-3.94 (m, 2H), 2.77 (s, 3H), 2.29-2.22 (m, 1H), 2.15-1.99 (m, 2H), 1.15 (d, 3H), 1.08-0.95 (m, 1H), 0.91-0.80 (m, 2H).

LC/MS (Method 1, ESIpos): Rₜ=1.72 min, m/z=325.10 [M+H]⁺.

### Example 483A

### 5-Methyl-3-[(1S,2S)-2-methylcyclopropyl]-2,4-dioxo-1-(3,3,3-trifluoropropyl)-1,2,3,4-tetrahydrothieno[2,3-d]pyrimidine-6-carbaldehyde

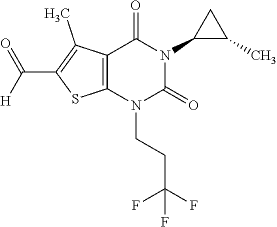 [SMILES: Cc1c(C=O)sc2c1c(=O)n([C@H]1C[C@@H]1C)c(=O)n2CCC(F)(F)F]

Analogously to the method described in Ex. 482A, 300 mg (1.14 mmol) of the compound from Ex. 303A and 399 μl (3.41 mmol) of 1,1,1-trifluoro-3-iodopropane were used to prepare 300 mg (73% of theory) of the title compound. The product was purified here by means of preparative HPLC (Method 11).

¹H-NMR (500 MHz, DMSO-d₆, δ/ppm): 10.09 (s, 1H), 4.13 (tq, 2H), 2.85-2.69 (m, 2H), 2.78 (s, 3H), 2.31-2.23 (m, 1H), 1.16 (d, 3H), 1.08-0.96 (m, 1H), 0.90-0.81 (m, 2H).

LC/MS (Method 2, ESIpos): Rₜ=1.02 min, m/z=361 [M+H]⁺.

### Example 484A

### 5-Methyl-3-[(S,2S)-2-methylcyclopropyl]-2,4-dioxo-1-(4,4-trifluorobutyl)-1,2,3,4-tetrahydrothieno[2,3-d]pyrimidine-6-carbaldehyde

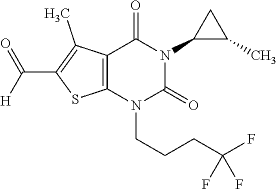 [SMILES: Cc1c(C=O)sc2c1c(=O)n([C@H]1C[C@@H]1C)c(=O)n2CCCC(F)(F)F]

To a solution of 300 mg (1.14 mmol) of the compound from Ex. 303A in 5 ml of anhydrous DMF were added 392 mg (2.84 mmol) of potassium carbonate, and the mixture was stirred at RT for 15 min. Then 810 mg (3.41 mmol) of 1,1,1-trifluoro-4-iodobutane were added, and the reaction mixture was stirred at 50° C. for about 16 h. After cooling to RT, the mixture was diluted with 200 ml of ethyl acetate and washed successively with water and saturated sodium chloride solution. After drying over anhydrous magnesium sulfate, the mixture was filtered and concentrated. The residue that remained was purified by means of MPLC (Biotage Isolera One, SNAP Ultra cartridge, 25 g of silica gel, cyclohexane/ethyl acetate gradient). After concentration of the product fraction and drying under high vacuum, 402 mg (91% of theory, 96% purity) of the title compound were obtained.

¹H-NMR (500 MHz, DMSO-d₆, δ/ppm): 10.08 (s, 1H), 4.04-3.91 (m, 2H), 2.77 (s, 3H), 2.48-2.37 (m, 2H), 2.25 (dt, 1H), 1.89 (quin, 2H), 1.15 (d, 3H), 1.07-0.97 (m, 1H), 0.90-0.80 (m, 2H).

LC/MS (Method 1, ESIpos): Rₜ=1.96 min, m/z=375.10 [M+H]⁺.

### Example 485A

### 1-[(3,3-Difluorocyclobutyl)methyl]-5-methyl-3-[(1S,2S)-2-methylcyclopropyl]-2,4-dioxo-1,2,3,4-tetrahydrothieno[2,3-d]pyrimidine-6-carbaldehyde

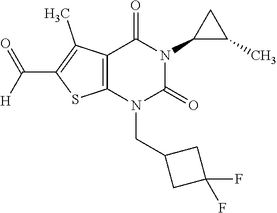 [SMILES: Cc1c(C=O)sc2c1c(=O)n([C@H]1C[C@@H]1C)c(=O)n2CC1CC(F)(F)C1]

Analogously to the method described in Ex. 484A, 300 mg (1.14 mmol) of the compound from Ex. 303A and 941 mg (3.41 mmol) of the compound from Ex. 316A were used to prepare 305 mg (68% of theory, 93% purity) of the title compound.

¹H-NMR (500 MHz, DMSO-d₆, δ/ppm): 10.08 (s, 1H), 4.16-4.01 (m, 2H), 2.77 (s, 3H), 2.73-2.57 (m, 3H), 2.56-2.46 (m, 2H, partially concealed by DMSO signal), 2.30-2.22 (m, 1H), 1.15 (d, 3H), 1.06-0.96 (m, 1H), 0.89-0.80 (m, 2H).

LC/MS (Method 1, ESIpos): Rₜ=1.92 min, m/z=369.11 [M+H]⁺.

### Example 486A

### 5-Methyl-3-[(1S,2S)-2-methylcyclopropyl]-2,4-dioxo-1-[(2R)-tetrahydrofuran-2-ylmethyl]-1,2,3,4-tetrahydrothieno[2,3-d]pyrimidine-6-carbaldehyde

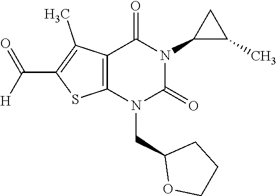 [SMILES: Cc1c(C=O)sc2c1c(=O)n([C@H]1C[C@@H]1C)c(=O)n2C[C@H]1CCCO1]

To a solution of 300 mg (1.14 mmol) of the compound from Ex. 303A in 6 ml of anhydrous DMF were added 392 mg (2.84 mmol) of potassium carbonate, and the mixture was stirred at RT for 15 min. Then 375 mg (2.27 mmol) of (2R)-2-(bromomethyl)tetrahydrofuran were added, and the reaction mixture was stirred at 50° C. After 3 days, a further 157 mg (1.13 mmol) of potassium carbonate and 187 mg (1.13 mmol) of (2R)-2-(bromomethyl)tetrahydrofuran were added and the stirring was continued at 50° C. After a further 3 days, the reaction mixture at RT was diluted with about 200 ml of ethyl acetate and washed successively with water and saturated sodium chloride solution. After drying over anhydrous magnesium sulfate, the mixture was filtered and concentrated. The remaining residue was purified by means of preparative HPLC (Method 11). After concentration of the product fraction and drying under high vacuum, 172 mg (43% of theory) of the title compound were obtained.

¹H-NMR (500 MHz, DMSO-d₆, δ/ppm): 10.07 (s, 1H), 4.25-4.17 (m, 1H), 4.12 (dd, 1H), 3.79-3.67 (m, 2H), 3.62 (td, 1H), 2.76 (s, 3H), 2.30-2.24 (m, 1H), 2.04-1.96 (m, 1H), 1.95-1.86 (m, 1H), 1.86-1.75 (m, 1H), 1.71-1.61 (m, 1H), 1.15 (d, 3H), 1.06-0.97 (m, 1H), 0.91-0.80 (m, 2H).

LC/MS (Method 1, ESIpos): Rₜ=1.76 min, m/z=349.12 [M+H]⁺.

### Example 487A

### tert-Butyl 2-({1-ethyl-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

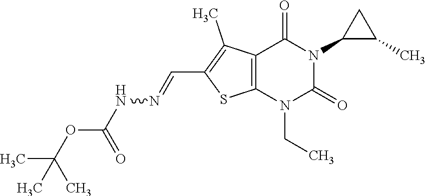 [SMILES: CCn1c(=O)n([C@H]2C[C@@H]2C)c(=O)c2c(C)c(C=NNC(=O)OC(C)(C)C)sc21]

Analogously to the method described in Ex. 471A, 346 mg (1.18 mmol, 95% purity) of the compound from Ex. 474A and 235 mg (1.78 mmol) of tert-butyl hydrazinecarboxylate were used to prepare 452 mg (95% of theory, 97% purity) of the title compound.

¹H-NMR (500 MHz, DMSO-d₆, δ/ppm): 10.84 (br. s, 1H), 8.28 (s, 1H), 3.96-3.85 (m, 2H), 2.43 (s, 3H), 2.23 (dt, 1H), 1.45 (s, 9H), 1.24 (t, 3H), 1.15 (d, 3H), 1.05-0.94 (m, 1H), 0.88-0.79 (m, 2H).

LC/MS (Method 2, ESIneg): Rₜ=1.07 min, m/z=405 [M−H].

### Example 488A

### tert-Butyl 2-({5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

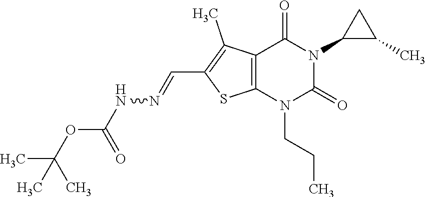 [SMILES: CCCn1c(=O)n([C@H]2C[C@@H]2C)c(=O)c2c(C)c(C=NNC(=O)OC(C)(C)C)sc21]

Analogously to the method described in Ex. 471A, 321 mg (1.05 mmol) of the compound from Ex. 475A and 208 mg (1.57 mmol) of tert-butyl hydrazinecarboxylate were used to prepare 396 mg (88% of theory) of the title compound.

¹H-NMR (500 MHz, DMSO-d₆, δ/ppm): 10.84 (br. s, 1H), 8.28 (s, 1H), 3.89-3.76 (m, 2H), 2.43 (s, 3H), 2.26-2.21 (m, 1H), 1.70 (sext, 2H), 1.45 (s, 9H), 1.15 (d, 3H), 1.04-0.95 (m, 1H), 0.92 (t, 3H), 0.87-0.79 (m, 2H).

LC/MS (Method 2, ESIpos): Rₜ=1.14 min, m/z=421 [M+H]⁺.

### Example 489A

### tert-Butyl 2-({1-butyl-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

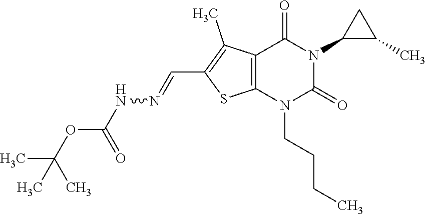 [SMILES: CCCCn1c(=O)n([C@H]2C[C@@H]2C)c(=O)c2c(C)c(C=NNC(=O)OC(C)(C)C)sc21]

Analogously to the method described in Ex. 471A, 345 mg (1.08 mmol) of the compound from Ex. 339A and 213 mg (1.62 mmol) of tert-butyl hydrazinecarboxylate were used to prepare 442 mg (89% of theory, 94% purity) of the title compound.

¹H-NMR (500 MHz, DMSO-d₆, δ/ppm): 10.85 (br. s, 1H), 8.28 (s, 1H), 3.92-3.79 (m, 2H), 2.44 (s, 3H), 2.27-2.21 (m, 1H), 1.66 (quin, 2H), 1.46 (s, 9H), 1.40-1.31 (m, 2H), 1.15 (d, 3H), 1.04-0.96 (m, 1H), 0.93 (t, 3H), 0.88-0.79 (m, 2H).

LC/MS (Method 1, ESIpos): Rₜ=2.29 min, m/z=435.21 [M+H]⁺.

### Example 490A

### tert-Butyl 2-({1-(2-fluoroethyl)-5-methyl-3-[(S,2S)-2-methylcyclopropyl]-2,4-dioxo-1,2,3,4-tetrahydrothieno[2,3-d]pyrimidin-6-yl}methylene)hydrazinecarboxylate

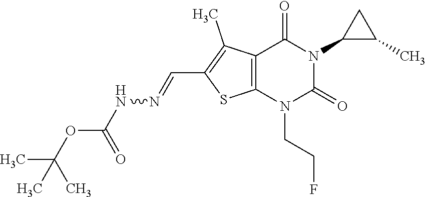 [SMILES: Cc1c(C=NNC(=O)OC(C)(C)C)sc2c1c(=O)n([C@H]1C[C@@H]1C)c(=O)n2CCF]

Analogously to the method described in Ex. 471A, 320 mg (0.990 mmol, 96% purity) of the compound from Ex. 477A and 204 mg (1.55 mmol) of tert-butyl hydrazinecarboxylate were used to prepare 420 mg (95% of theory, 96% purity) of the title compound.

¹H-NMR (500 MHz, DMSO-d₆, δ/ppm): 10.84 (br. s, 1H), 8.27 (s, 1H), 4.73 (dt, 2H), 4.20 (dm, 2H), 2.43 (s, 3H), 2.25 (dt, 1H), 1.45 (s, 9H), 1.15 (d, 3H), 1.02 (tq, 1H), 0.91-0.79 (m, 2H).

LC/MS (Method 1, ESIneg): Rₜ=1.07 min, m/z=423.15 [M−H].

### Example 491A

### tert-Butyl 2-({1-(2,2-difluoroethyl)-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

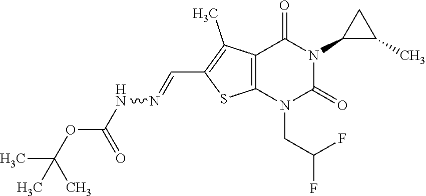 [SMILES: Cc1c(C=NNC(=O)OC(C)(C)C)sc2c1c(=O)n([C@H]1C[C@@H]1C)c(=O)n2CC(F)F]

Analogously to the method described in Ex. 471A, 330 mg (0.945 mmol, 94% purity) of the compound from Ex. 479A and 199 mg (1.51 mmol) of tert-butyl hydrazinecarboxylate were used to prepare 412 mg (78% of theory, 80% purity) of the title compound.

LC/MS (Method 1, ESIneg): Rₜ=2.01 min, m/z=441.14 [M−H].

### Example 492A

### tert-Butyl 2-({5-methyl-3-[(1S,2S)-2-methylcyclopropyl]-2,4-dioxo-1-(2,2,2-trifluoroethyl)-1,2,3,4-tetrahy drothieno[2,3-d]pyrimidin-6-yl}methylene)hydrazinecarboxylate

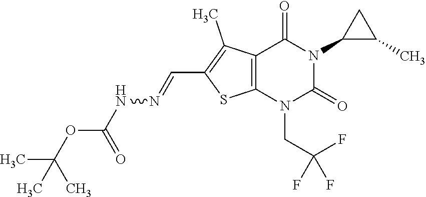 [SMILES: Cc1c(C=NNC(=O)OC(C)(C)C)sc2c1c(=O)n([C@H]1C[C@@H]1C)c(=O)n2CC(F)(F)F]

Analogously to the method described in Ex. 471A, 180 mg (0.494 mmol, 95% purity) of the compound from Ex. 481A and 103 mg (0.780 mmol) of tert-butyl hydrazinecarboxylate were used to prepare 220 mg (82% of theory, 85% purity) of the title compound.

¹H-NMR (500 MHz, DMSO-d₆, δ/ppm): 10.88 (br. s, 1H), 8.28 (s, 1H), 4.95-4.76 (m, 2H), 2.44 (s, 3H), 2.32-2.25 (m, 1H), 1.45 (s, 9H), 1.15 (d, 3H), 1.05-0.96 (m, 1H), 0.90-0.80 (m, 2H).

LC/MS (Method 1, ESIneg): Rₜ=2.13 min, m/z=459.13 [M−H]⁻.

### Example 493A

### tert-Butyl 2-({1-(3-fluoropropyl)-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

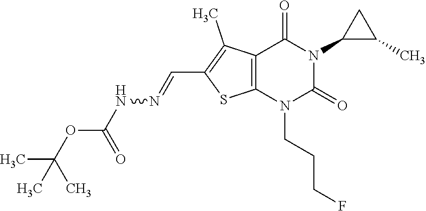 [SMILES: Cc1c(C=NNC(=O)OC(C)(C)C)sc2c1c(=O)n([C@H]1C[C@@H]1C)c(=O)n2CCCF]

Analogously to the method described in Ex. 471A, 345 mg (1.06 mmol) of the compound from Ex. 482A and 211 mg (1.60 mmol) of tert-butyl hydrazinecarboxylate were used to prepare 449 mg (92% of theory, 96% purity) of the title compound.

¹H-NMR (500 MHz, DMSO-d₆, δ/ppm): 10.84 (br. s, 1H), 8.28 (s, 1H), 4.55 (dt, 2H), 4.04-3.92 (m, 2H), 2.44 (s, 3H), 2.26-2.21 (m, 1H), 2.14-2.01 (m, 2H), 1.46 (s, 9H), 1.15 (d, 3H), 1.05-0.95 (m, 1H), 0.90-0.78 (m, 2H).

LC/MS (Method 1, ESIneg): Rₜ=2.01 min, m/z=437.17 [M−H].

### Example 494A

### tert-Butyl 2-({5-methyl-3-[(1S,2S)-2-methylcyclopropyl]-2,4-dioxo-1-(3,3,3-trifluoropropyl)-1,2,3,4-tetrahy drothieno[2,3-d]pyrimidin-6-yl}methylene)hydrazinecarboxylate

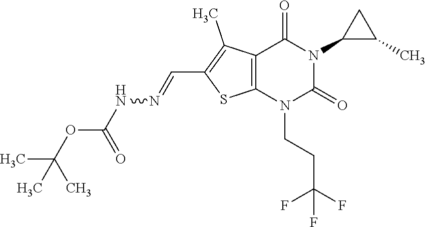 [SMILES: Cc1c(C=NNC(=O)OC(C)(C)C)sc2c1c(=O)n([C@H]1C[C@@H]1C)c(=O)n2CCC(F)(F)F]

Analogously to the method described in Ex. 471A, 300 mg (0.833 mmol) of the compound from Ex. 483A and 165 mg (1.25 mmol) of tert-butyl hydrazinecarboxylate were used to prepare 378 mg (90% of theory, 95% purity) of the title compound.

¹H-NMR (500 MHz, DMSO-d₆, δ/ppm): 10.87 (br. s, 1H), 8.29 (s, 1H), 4.11 (tq, 2H), 2.77 (qt, 2H), 2.45 (s, 3H), 2.28-2.21 (m, 1H), 1.46 (s, 9H), 1.16 (d, 3H), 1.06-0.95 (m, 1H), 0.89-0.80 (m, 2H).

LC/MS (Method 1, ESIneg): Rₜ=2.15 min, m/z=473.15 [M−H].

### Example 495A

### tert-Butyl 2-({5-methyl-3-[(1S,2S)-2-methylcyclopropyl]-2,4-dioxo-1-(4,4,4-trifluorobutyl)-1,2,3,4-tetrahy drothieno[2,3-d]pyrimidin-6-yl}methylene)hydrazinecarboxylate

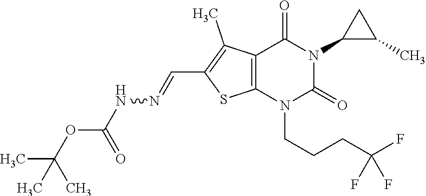 [SMILES: Cc1c(C=NNC(=O)OC(C)(C)C)sc2c1c(=O)n([C@H]1C[C@@H]1C)c(=O)n2CCCC(F)(F)F]

Analogously to the method described in Ex. 471A, 395 mg (1.06 mmol) of the compound from Ex. 484A and 209 mg (1.58 mmol) of tert-butyl hydrazinecarboxylate were used to prepare 484 mg (88% of theory, 94% purity) of the title compound.

¹H-NMR (500 MHz, DMSO-d₆, δ/ppm): 10.85 (br. s, 1H), 8.29 (s, 1H), 4.03-3.89 (m, 2H), 2.49-2.37 (m, 2H), 2.44 (s, 3H), 2.23 (dt, 1H), 1.90 (quin, 2H), 1.46 (s, 9H), 1.15 (d, 3H), 1.05-0.96 (m, 1H), 0.90-0.79 (m, 2H).

LC/MS (Method 1, ESIneg): Rₜ=2.22 min, m/z=487.16 [M−H].

### Example 496A

### tert-Butyl 2-({1-(2-methoxyethyl)-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

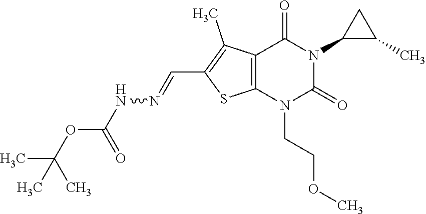 [SMILES: COCCn1c(=O)n([C@H]2C[C@@H]2C)c(=O)c2c(C)c(C=NNC(=O)OC(C)(C)C)sc21]

Analogously to the method described in Ex. 471A, 325 mg (1.01 mmol) of the compound from Ex. 350A and 200 mg (1.51 mmol) of tert-butyl hydrazinecarboxylate were used to prepare 391 mg (85% of theory, 96% purity) of the title compound.

¹H-NMR (500 MHz, DMSO-d₆, δ/ppm): 10.84 (br. s, 1H), 8.27 (s, 1H), 4.09-3.97 (m, 2H), 3.63 (t, 2H), 3.25 (s, 3H), 2.43 (s, 3H), 2.28-2.21 (m, 1H), 1.46 (s, 9H), 1.15 (d, 3H), 1.01 (tq, 1H), 0.89-0.79 (m, 2H).

LC/MS (Method 1, ESIpos): Rₜ=1.94 min, m/z=437.18 [M+H]⁺.

### Example 497A

### tert-Butyl 2-({1-[(2,2-difluorocyclopropyl)methyl]-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate (diastereomer mixture)

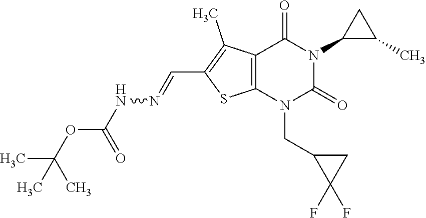 [SMILES: Cc1c(C=NNC(=O)OC(C)(C)C)sc2c1c(=O)n([C@H]1C[C@@H]1C)c(=O)n2CC1CC1(F)F]

Analogously to the method described in Ex. 471A, 325 mg (0.917 mmol) of the compound from Ex. 346A and 182 mg (1.38 mmol) of tert-butyl hydrazinecarboxylate were used to prepare 400 mg (93% of theory) of the title compound.

¹H-NMR (500 MHz, DMSO-d₆, δ/ppm): 10.84 (br. s, 1H), 8.29 (br. s, 1H), 4.19-4.06 (m, 1H), 4.00-3.88 (m, 1H), 2.44 (s, 3H), 2.29-2.15 (m, 2H), 1.76-1.64 (m, 1H), 1.60-1.30 (m, 1H), 1.45 (s, 9H), 1.15 (d, 3H), 1.06-0.96 (m, 1H), 0.89-0.78 (m, 2H).

LC/MS (Method 1, ESIpos): Rₜ=2.11 min, m/z=469.17 [M+H]⁺.

### Example 498A

### tert-Butyl 2-({1-(cyclobutylmethyl)-5-methyl-3-[(1S,2S)-2-methylcyclopropyl]-2,4-dioxo-1,2,3,4-tetrahydrothieno[2,3-d]pyrimidin-6-yl}methylene)hydrazinecarboxylate

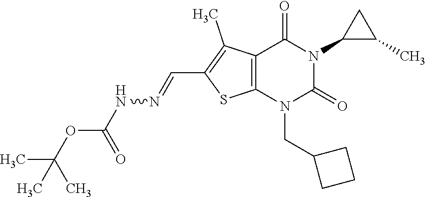 [SMILES: Cc1c(C=NNC(=O)OC(C)(C)C)sc2c1c(=O)n([C@H]1C[C@@H]1C)c(=O)n2CC1CCC1]

Analogously to the method described in Ex. 471A, 325 mg (0.978 mmol) of the compound from Ex. 345A and 194 mg (1.47 mmol) of tert-butyl hydrazinecarboxylate were used to prepare 389 mg (89% of theory) of the title compound.

¹H-NMR (500 MHz, DMSO-d₆, δ/ppm): 10.85 (br. s, 1H), 8.28 (s, 1H), 3.93 (tt, 2H), 2.84-2.71 (m, 1H), 2.43 (s, 3H), 2.24 (td, 1H), 2.05-1.92 (m, 2H), 1.89-1.75 (m, 4H), 1.46 (s, 9H), 1.15 (d, 3H), 1.04-0.91 (m, 1H), 0.83 (dd, 2H).

LC/MS (Method 1, ESIpos): Rₜ=2.34 min, m/z=447.20 [M+H]⁺.

### Example 499A

### tert-Butyl 2-({1-[(3,3-difluorocyclobutyl)methy]-5-methy-5-3-[(-methylcyclopropyl]-2,4-dioxo-1,2,3,4-tetrahydrothieno[2,3-d]pyrimidin-6-yl}methylene)hydrazinecarboxylate

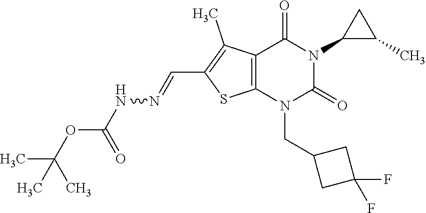 [SMILES: Cc1c(C=NNC(=O)OC(C)(C)C)sc2c1c(=O)n([C@H]1C[C@@H]1C)c(=O)n2CC1CC(F)(F)C1]

Analogously to the method described in Ex. 471A, 300 mg (0.757 mmol, 93% purity) of the compound from Ex. 485A and 161 mg (1.22 mmol) of tert-butyl hydrazinecarboxylate were used to prepare 342 mg (86% of theory, 92% purity) of the title compound.

¹H-NMR (500 MHz, DMSO-d₆, δ/ppm): 10.85 (m, 1H), 8.28 (s, 1H), 4.13-3.99 (m, 2H), 2.76-2.58 (m, 3H), 2.56-2.47 (m, 2H, partially concealed by DMSO signal), 2.44 (s, 3H), 2.28-2.20 (m, 1H), 1.46 (s, 9H), 1.15 (d, 3H), 1.06-0.94 (m, 1H), 0.88-0.80 (m, 2H).

LC/MS (Method 1, ESIneg): Rₜ=2.20 min, m/z=481.17 [M−H].

### Example 500A

### tert-Butyl 2-({5-methyl-3-[(1S,2S)-2-methylcyclopropyl]-2,4-dioxo-1-[(2R)-tetrahydrofuran-2-ylmethyl]-1,2,3,4-tetrahydrothieno[2,3-d]pyrimidin-6-yl}methylene)hydrazinecarboxylate

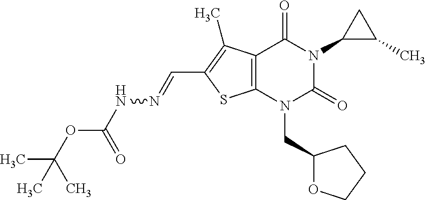 [SMILES: Cc1c(C=NNC(=O)OC(C)(C)C)sc2c1c(=O)n([C@H]1C[C@@H]1C)c(=O)n2C[C@H]1CCCO1]

To a solution of 170 mg (0.488 mmol) of the compound from Ex. 486A in 5 ml of ethanol were added 97 mg (0.732 mmol) of tert-butyl hydrazinecarboxylate and 3 drops of conc. hydrochloric acid. After the reaction mixture had been stirred at RT for about 16 h, it was concentrated almost to dryness, then 150 ml of cold water were added and then the mixture was neutralized by adding saturated sodium hydrogencarbonate solution. In the course of this, the product precipitated out. The product was filtered off with suction, washed with a little water and dried under high vacuum. 180 mg (71% of theory, 90% purity) of the title compound were obtained.

¹H-NMR (500 MHz, DMSO-d₆, δ/ppm): 10.84 (br. s, 1H), 8.28 (br. s, 1H), 4.26-4.15 (m, 1H), 4.14-4.01 (m, 1H), 3.82-3.57 (m, 3H), 2.43 (s, 3H), 2.30-2.19 (m, 1H), 2.05-1.76 (m, 3H), 1.71-1.60 (m, 1H), 1.46 (s, 9H), 1.15 (br. d, 3H), 1.00 (br. d, 1H), 0.85 (br. d, 2H).

LC/MS (Method 1 ESIneg): Rₜ=2.04 min, m/z=461.19 [M−H].

### Example 501A

### tert-Butyl 2-({1-ethyl-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

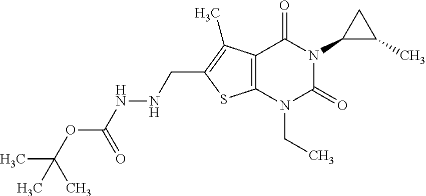 [SMILES: CCn1c(=O)n([C@H]2C[C@@H]2C)c(=O)c2c(C)c(CNNC(=O)OC(C)(C)C)sc21]

Analogously to the method described in Ex. 472A, 452 mg (1.08 mmol, 97% purity) of the compound from Ex. 487A and 346 mg (5.39 mmol) of sodium cyanoborohydride were used to prepare 339 mg (70% of theory, 91% purity) of the title compound. The MPLC purification was effected here using a cartridge with 25 g of silica gel and cyclohexane/ethyl acetate 2:1 as eluent.

¹H-NMR (500 MHz, DMSO-d₆, δ/ppm): 8.24 (br. s, 1H), 4.99 (br. s, 1H), 3.97 (br. d, 2H), 3.92-3.82 (m, 2H), 2.31 (s, 3H), 2.26-2.21 (m, 1H), 1.39 (s, 9H), 1.24 (t, 3H), 1.16 (d, 3H), 1.03-0.92 (m, 1H), 0.87-0.79 (m, 2H).

LC/MS (Method 1, ESIpos): Rₜ=1.88 min, m/z=277.10 [M+H−C₅H₁₂N₂O₂]⁺.

### Example 502A

### tert-Butyl 2-({5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

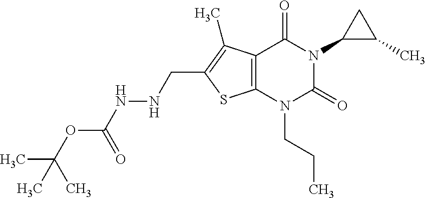 [SMILES: CCCn1c(=O)n([C@H]2C[C@@H]2C)c(=O)c2c(C)c(CNNC(=O)OC(C)(C)C)sc21]

Analogously to the method described in Ex. 472A, 396 mg (0.923 mmol) of the compound from Ex. 488A and 296 mg (4.61 mmol) of sodium cyanoborohydride were used to prepare 345 mg (77% of theory, 88% purity) of the title compound. The MPLC purification was effected here using a cartridge with 25 g of silica gel and cyclohexane/ethyl acetate 2:1 as eluent.

¹H-NMR (500 MHz, DMSO-d₆, δ/ppm): 8.24 (br. s, 1H), 4.99 (br. d, 1H), 3.96 (br. d, 2H), 3.86-3.73 (m, 2H), 2.31 (s, 3H), 2.27-2.20 (m, 1H), 1.70 (sext, 2H), 1.39 (s, 9H), 1.15 (d, 3H), 0.97 (dtd, 1H), 0.91 (t, 3H), 0.86-0.78 (m, 2H).

LC/MS (Method 1, ESIpos): Rₜ=2.03 min, m/z=291.12 [M+H−C₅H₁₂N₂O₂]⁺.

### Example 503A

### tert-Butyl 2-({1-butyl-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

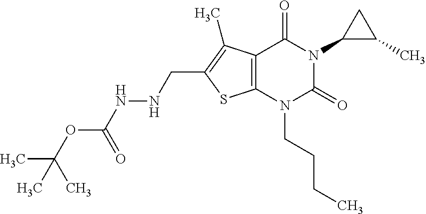 [SMILES: CCCCn1c(=O)n([C@H]2C[C@@H]2C)c(=O)c2c(C)c(CNNC(=O)OC(C)(C)C)sc21]

Analogously to the method described in Ex. 472A, 440 mg (0.962 mmol, 94% purity) of the compound from Ex. 489A and 308 mg (4.81 mmol) of sodium cyanoborohydride were used to prepare 366 mg (78% of theory, 90% purity) of the title compound. The MPLC purification was effected here using a cartridge with 25 g of silica gel and cyclohexane/ethyl acetate 2:1 as eluent.

¹H-NMR (500 MHz, DMSO-d₆, δ/ppm): 8.23 (br. s, 1H), 4.98 (br. d, 1H), 3.96 (br. d, 2H), 3.89-3.75 (m, 2H), 2.31 (s, 3H), 2.26-2.20 (m, 1H), 1.65 (quin, 2H), 1.38 (s, 9H), 1.38-1.30 (m, 2H), 1.15 (d, 3H), 1.00-0.94 (m, 1H), 0.92 (t, 3H), 0.85-0.78 (m, 2H).

LC/MS (Method 1, ESIpos): Rₜ=2.18 min, m/z=305.13 [M+H−C₅H₁₂N₂O₂]⁺.

### Example 504A

### tert-Butyl 2-({1-(2-fluoroethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

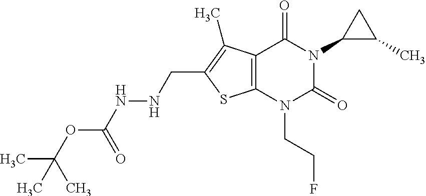 [SMILES: Cc1c(CNNC(=O)OC(C)(C)C)sc2c1c(=O)n([C@H]1C[C@@H]1C)c(=O)n2CCF]

To a solution of 415 mg (0.939 mmol, 96% purity) of the compound from Ex. 490A in 20 ml of methanol were added 307 mg (4.89 mmol) of sodium cyanoborohydride and a little Bromocresol Green. Subsequently, a sufficient amount of acetic acid was added by titration that the indicator colour just changed from blue to yellow. Then the reaction mixture was heated to 65° C. After 1 h, a further 154 mg (2.44 mmol) of sodium cyanoborohydride were added. Over the entire reaction time, by addition of further acetic acid, the pH was constantly regulated such that the indicator colour just remained yellow. After a total reaction time of 3 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 25 g of silica gel, eluent: cyclohexane/ethyl acetate 2:1). The product fractions were combined and concentrated. After drying under high vacuum, 380 mg (85% of theory, 90% purity) of the title compound were obtained.

LC/MS (Method 1, ESIpos): Rₜ=1.77 min, m/z=295.09 [M+H−C₅H₁₂N₂O₂]⁺.

### Example 505A

### tert-Butyl 2-({1-(2,2-difluoroethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

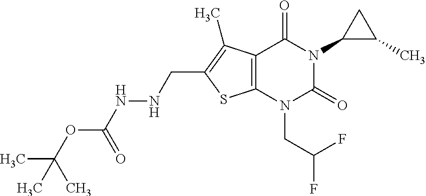 [SMILES: Cc1c(CNNC(=O)OC(C)(C)C)sc2c1c(=O)n([C@H]1C[C@@H]1C)c(=O)n2CC(F)F]

Analogously to the method described in Ex. 504A, 400 mg (0.723 mmol, 80% purity) of the compound from Ex. 491A and a total of 341 mg (5.42 mmol) of sodium cyanoborohydride were used to prepare 205 mg (63% of theory, 72% purity) of the title compound.

LC/MS (Method 1, ESIpos): Rₜ=1.88 min, m/z=313.08 [M+H−C₅H₁₂N₂O₂]⁺.

### Example 506A

### tert-Butyl 2-({5-methyl-3-[(1S,2S)-2-methylcyclopropyl]-2,4-dioxo-1-(2,2,2-trifluoroethyl)-1,2,3,4-tetrahy drothieno[2,3-d]pyrimidin-6-yl}methyl)hydrazinecarboxylate

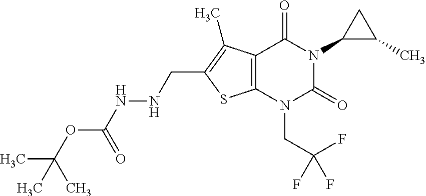 [SMILES: Cc1c(CNNC(=O)OC(C)(C)C)sc2c1c(=O)n([C@H]1C[C@@H]1C)c(=O)n2CC(F)(F)F]

Analogously to the method described in Ex. 504A, 218 mg (0.402 mmol, 85% purity) of the compound from Ex. 492A and a total of 223 mg (3.55 mmol) of sodium cyanoborohydride were used to prepare 170 mg (79% of theory, 87% purity) of the title compound.

LC/MS (Method 1, ESIpos): Rₜ=2.00 min, m/z=331.07 [M+H−C₅H₁₂N₂O₂]⁺.

### Example 507A

### tert-Butyl 2-({1-(3-fluoropropyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

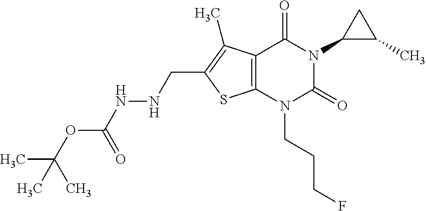 [SMILES: Cc1c(CNNC(=O)OC(C)(C)C)sc2c1c(=O)n([C@H]1C[C@@H]1C)c(=O)n2CCCF]

Analogously to the method described in Ex. 472A, 444 mg (0.972 mmol, 96% purity) of the compound from Ex. 493A and 312 mg (4.86 mmol) of sodium cyanoborohydride were used to prepare 356 mg (73% of theory, 88% purity) of the title compound. The MPLC purification was effected here using a cartridge with 25 g of silica gel and cyclohexane/ethyl acetate 2:1 as eluent.

¹H-NMR (500 MHz, DMSO-d₆, δ/ppm): 8.24 (br. s, 1H), 4.99 (br. d, 1H), 4.54 (dt, 2H), 4.01-3.86 (m, 4H), 2.31 (s, 3H), 2.26-2.20 (m, 1H), 2.13-2.00 (m, 2H), 1.38 (s, 9H), 1.15 (d, 3H), 1.04-0.92 (m, 1H), 0.82 (dd, 2H).

LC/MS (Method 1, ESIpos): Rₜ=1.89 min, m/z=309.11 [M+H−C₅H₁₂N₂O₂]⁺.

### Example 508A

### tert-Butyl 2-({5-methyl-3-[(1S,2S)-2-methylcyclopropyl]-2,4-dioxo-1-(3,3,3-trifluoropropyl)-1,2,3,4-tetrahy drothieno[2,3-d]pyrimidin-6-yl}methyl)hydrazinecarboxylate

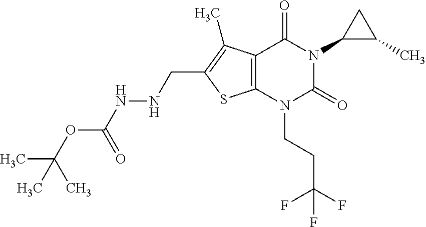 [SMILES: Cc1c(CNNC(=O)OC(C)(C)C)sc2c1c(=O)n([C@H]1C[C@@H]1C)c(=O)n2CCC(F)(F)F]

Analogously to the method described in Ex. 472A, 372 mg (0.745 mmol, 95% purity) of the compound from Ex. 494A and 239 mg (3.72 mmol) of sodium cyanoborohydride were used to prepare 323 mg (79% of theory, 87% purity) of the title compound. The MPLC purification was effected here using a cartridge with 25 g of silica gel and cyclohexane/ethyl acetate 2:1 as eluent.

¹H-NMR (500 MHz, DMSO-d₆, δ/ppm): 8.24 (br. s, 1H), 5.04 (br. d, 1H), 4.14-4.00 (m, 2H), 3.97 (br. d, 2H), 2.82-2.67 (m, 2H), 2.31 (s, 3H), 2.27-2.20 (m, 1H), 1.38 (s, 9H), 1.15 (d, 3H), 0.98 (qdd, 1H), 0.89-0.78 (m, 2H).

LC/MS (Method 1, ESIpos): Rₜ=2.06 min, m/z=345.09 [M+H−C₅H₁₂N₂O₂]⁺.

### Example 509A

### tert-Butyl 2-({5-methyl-3-[(1S,2S)-2-methylcyclopropyl]-2,4-dioxo-1-(4,4,4-trifluorobutyl)-1,2,3,4-tetrahy drothieno[2,3-d]pyrimidin-6-yl}methyl)hydrazinecarboxylate

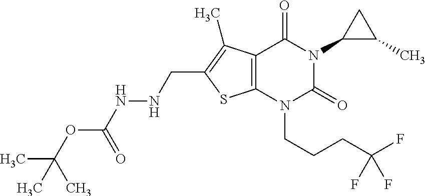 [SMILES: Cc1c(CNNC(=O)OC(C)(C)C)sc2c1c(=O)n([C@H]1C[C@@H]1C)c(=O)n2CCCC(F)(F)F]

Analogously to the method described in Ex. 472A, 484 mg (0.931 mmol, 94% purity) of the compound from Ex. 495A and 299 mg (4.66 mmol) of sodium cyanoborohydride were used to prepare 380 mg (66% of theory, 80% purity) of the title compound. The MPLC purification was effected here using a cartridge with 25 g of silica gel and cyclohexane/ethyl acetate 2:1 as eluent.

LC/MS (Method 1, ESIpos): Rₜ=2.11 min, m/z=359.10 [M+H−C₅H₁₂N₂O₂]⁺.

### Example 510A

### tert-Butyl 2-({1-(2-methoxyethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

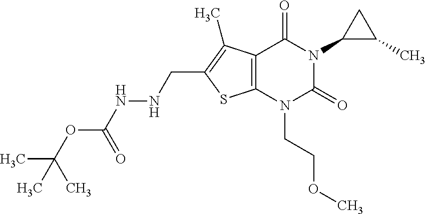 [SMILES: COCCn1c(=O)n([C@H]2C[C@@H]2C)c(=O)c2c(C)c(CNNC(=O)OC(C)(C)C)sc21]

To a solution of 390 mg (0.893 mmol) of the compound from Ex. 496A in 20 ml of methanol were added 281 mg (4.47 mmol) of sodium cyanoborohydride and a little Bromocresol Green.

Subsequently, a sufficient amount of acetic acid was added by titration that the indicator colour just changed from blue to yellow. Then the reaction mixture was heated to 65° C. After 1 h, a further 140 mg (2.23 mmol) of sodium cyanoborohydride were added and, after a further hour, another 281 mg (4.47 mmol). Over the entire reaction time, by addition of further acetic acid, the pH was constantly regulated such that the indicator colour just remained yellow. After a total reaction time of 4 h, the volatile constituents of the reaction mixture were substantially removed on a rotary evaporator. The remaining residue was taken up in ethyl acetate and washed successively with saturated aqueous sodium hydrogencarbonate solution, water and saturated aqueous sodium chloride solution. After drying over anhydrous magnesium sulfate, the mixture was filtered and concentrated to dryness. The crude product was purified by means of MPLC (Biotage Isolera One, SNAP Ultra cartridge, 10 g of silica gel, eluent: cyclohexane/ethyl acetate 2:1). The product fractions were combined and concentrated. After drying under high vacuum, 308 mg (77% of theory, 98% purity) of the title compound were obtained.

¹H-NMR (500 MHz, DMSO-d₆, δ/ppm): 8.23 (br. s, 1H), 4.95 (br. d, 1H), 4.06-3.90 (m, 4H), 3.62 (t, 2H), 3.25 (s, 3H), 2.30 (s, 3H), 2.26-2.21 (m, 1H), 1.39 (s, 9H), 1.15 (d, 3H), 1.03-0.93 (m, 1H), 0.86-0.78 (m, 2H).

LC/MS (Method 1, ESIpos): Rₜ=1.81 min, m/z=307.11 [M+H−C₅H₁₂N₂O₂]⁺.

### Example 511A

### tert-Butyl 2-({1-[(2,2-difluorocyclopropyl)methyl]-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate (diastereomer mixture)

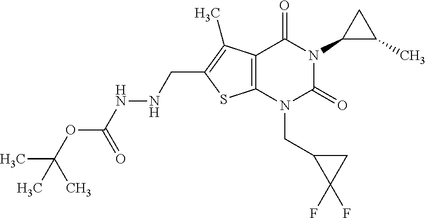 [SMILES: Cc1c(CNNC(=O)OC(C)(C)C)sc2c1c(=O)n([C@H]1C[C@@H]1C)c(=O)n2CC1CC1(F)F]

Analogously to the method described in Ex. 504A, 395 mg (0.843 mmol) of the compound from Ex. 497A and a total of 397 mg (6.32 mmol) of sodium cyanoborohydride were used to prepare 348 mg (76% of theory, 87% purity) of the title compound.

¹H-NMR (500 MHz, DMSO-d₆, δ/ppm): 8.24 (br. s, 1H), 5.01 (br. s, 1H), 4.09-3.88 (m, 4H), 2.31 (s, 3H), 2.28-2.13 (m, 2H), 1.74-1.63 (m, 1H), 1.52-1.43 (m, 1H), 1.38 (s, 9H), 1.15 (d, 3H), 1.04-0.93 (m, 1H), 0.83 (dd, 2H).

LC/MS (Method 1, ESIpos): Rₜ=2.04 min, m/z=339.10 [M+H−C₅H₁₂N₂O₂]⁺.

### Example 512A

### tert-Butyl 2-({1-(cyclobutylmethyl)-5-methyl-3-[(1 S,2S)-2-methylcyclopropyl]-2,4-dioxo-1,2,3,4-tetrahydrothieno[2,3-d]pyrimidin-6-yl}methyl)hydrazinecarboxylate

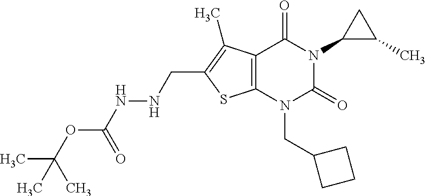 [SMILES: Cc1c(CNNC(=O)OC(C)(C)C)sc2c1c(=O)n([C@H]1C[C@@H]1C)c(=O)n2CC1CCC1]

Analogously to the method described in Ex. 472A, 389 mg (0.871 mmol) of the compound from Ex. 498A and 279 mg (4.36 mmol) of sodium cyanoborohydride were used to prepare 270 mg (60% of theory, 87% purity) of the title compound. The MPLC purification was effected here using a cartridge with 25 g of silica gel and cyclohexane/ethyl acetate 2:1 as eluent.

LC/MS (Method 1, ESIpos): Rₜ=2.23 min, m/z=317.13 [M+H−C₅H₁₂N₂O₂]⁺.

### Example 513A

### tert-Butyl 2-({1-[(3,3-difluorocyclobutyl)methyl]-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

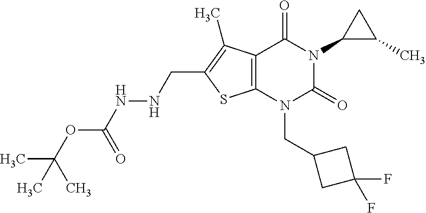 [SMILES: Cc1c(CNNC(=O)OC(C)(C)C)sc2c1c(=O)n([C@H]1C[C@@H]1C)c(=O)n2CC1CC(F)(F)C1]

Analogously to the method described in Ex. 510A, 430 mg (0.649 mmol, 92% purity) of the compound from Ex. 499A and a total of 553 mg (8.81 mmol) of sodium cyanoborohydride were used to prepare 325 mg (90% of theory, 87% purity) of the title compound.

¹H-NMR (500 MHz, DMSO-d₆, δ/ppm): 8.24 (br. s, 1H), 5.01 (br. d, 1H), 4.09-3.91 (m, 4H), 2.73-2.59 (m, 3H), 2.56-2.47 (m, 2H), 2.30 (s, 3H), 2.27-2.20 (m, 1H), 1.38 (s, 9H), 1.15 (d, 3H), 0.97 (dtt, 1H), 0.86-0.78 (m, 2H).

LC/MS (Method 1, ESIpos): Rₜ=2.09 min, m/z=353.11 [M+H−C₅H₁₂N₂O₂]⁺.

### Example 514A

### tert-Butyl 2-({5-methyl-3-[(1S,2S)-2-methylcyclopropyl]-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

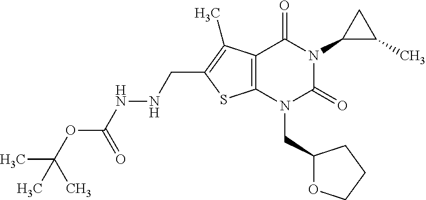 [SMILES: Cc1c(CNNC(=O)OC(C)(C)C)sc2c1c(=O)n([C@H]1C[C@@H]1C)c(=O)n2C[C@H]1CCCO1]

Analogously to the method described in Ex. 504A, 178 mg (0.347 mmol, 90% purity) of the compound from Ex. 500A and a total of 181 mg (2.89 mmol) of sodium cyanoborohydride were used to prepare 110 mg (47% of theory, 70% purity) of the title compound.

LC/MS (Method 1, ESIpos): Rₜ=1.88 min, m/z=333.13 [M+H−C₅H₁₂N₂O₂]⁺.

### Example 515A

### tert-Butyl 2-carbamoyl-2-({1-ethyl-5-methyl-3-[(1S,2S)-2-methylcyclopropyl]-2,4-dioxo-1,2,3,4-tetrahy drothieno[2,3-d]pyrimidin-6-yl}methyl)hydrazinecarboxylate

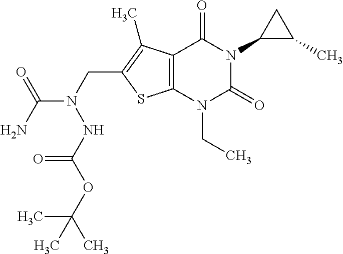 [SMILES: CCn1c(=O)n([C@H]2C[C@@H]2C)c(=O)c2c(C)c(CN(NC(=O)OC(C)(C)C)C(N)=O)sc21]

To a solution of 339 mg (0.788 mmol, 95% purity) of the compound from Ex. 501A in 20 ml of isopropanol were added 211 μl (1.58 mmol) of trimethylsilyl isocyanate, and the mixture was stirred at RT for about 18 h. Thereafter, the reaction mixture was concentrated. The residue that remained was purified by means of MPLC (Biotage Isolera One, SNAP Ultra cartridge, 25 g of silica gel, cyclohexane/ethyl acetate 1:1→dichloromethane/methanol 10:1). After concentration of the product fraction and drying under high vacuum, 288 mg (71% of theory, 88% purity) of the title compound were obtained.

¹H-NMR (500 MHz, DMSO-d₆, δ/ppm): 8.90 (br. s, 1H), 6.17 (br. s, 2H), 4.94-4.25 (br. m, 2H), 3.92-3.81 (m, 2H), 2.32 (s, 3H), 2.25 (dt, 1H), 1.38 (br. s, 9H), 1.22 (t, 3H), 1.15 (d, 3H), 1.00-0.91 (m, 1H), 0.87-0.77 (m, 2H).

LC/MS (Method 1, ESIpos): Rₜ=1.57 min, m/z=452.20 [M+H]⁺.

### Example 516A

### tert-Butyl 2-carbamoyl-2-({5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

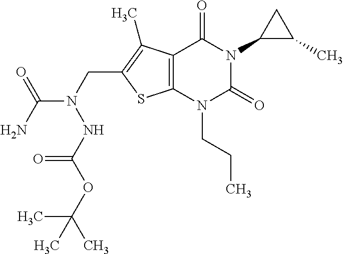 [SMILES: CCCn1c(=O)n([C@H]2C[C@@H]2C)c(=O)c2c(C)c(CN(NC(=O)OC(C)(C)C)C(N)=O)sc21]

Analogously to the method described in Ex. 473A, 345 mg (0.735 mmol, 90% purity) of the compound from Ex. 502A and 197 μl (1.47 mmol) of trimethylsilyl isocyanate were used to prepare 323 mg (85% of theory, 91% purity) of the title compound.

¹H-NMR (500 MHz, DMSO-d₆, δ/ppm): 8.90 (br. s, 1H), 6.17 (br. s, 2H), 4.97-4.22 (m, 2H), 3.85-3.73 (m, 2H), 2.32 (s, 3H), 2.27-2.22 (m, 1H), 1.68 (sext, 2H), 1.38 (br. s, 9H), 1.15 (d, 3H), 0.99-0.91 (m, 1H), 0.90 (t, 3H), 0.86-0.76 (m, 2H).

LC/MS (Method 1, ESIpos): Rₜ=1.69 min, m/z=466.21 [M+H]⁺.

### Example 517A

### tert-Butyl 2-({1-butyl-5-methyl-3-[(1S,2S)-2-methylcyclopropyl]-2,4-dioxo-1,2,3,4-tetrahydrothieno[2,3-d]pyrimidin-6-yl}methyl)-2-carbamoylhydrazinecarboxylate

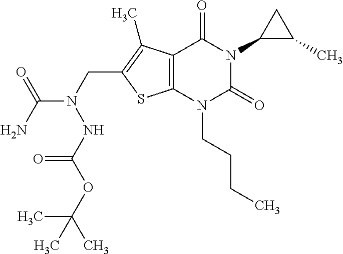 [SMILES: CCCCn1c(=O)n([C@H]2C[C@@H]2C)c(=O)c2c(C)c(CN(NC(=O)OC(C)(C)C)C(N)=O)sc21]

Analogously to the method described in Ex. 515A, 364 mg (0.792 mmol, 95% purity) of the compound from Ex. 503A and 212 μl (1.58 mmol) of trimethylsilyl isocyanate were used to prepare 286 mg (95% of theory, 94% purity) of the title compound.

¹H-NMR (500 MHz, DMSO-d₆, δ/ppm): 8.90 (br. s, 1H), 6.18 (br. s, 2H), 4.99-4.14 (m, 2H), 3.88-3.75 (m, 2H), 2.32 (s, 3H), 2.25 (dt, 1H), 1.64 (quin, 2H), 1.40 (s, 9H), 1.37-1.30 (m, 2H), 1.15 (d, 3H), 0.98-0.92 (m, 1H), 0.91 (t, 3H), 0.87-0.76 (m, 2H).

LC/MS (Method 1, ESIpos): Rₜ=1.79 min, m/z=480.23 [M+H]⁺.

### Example 518A

### tert-Butyl 2-carbamoyl-2-({1-(2-fluoroethyl)-5-methyl-3-[(1S,2S)-2-methylcyclopropyl]-2,4-dioxo-1,2,3,4-tetrahy drothieno[2,3-d]pyrimidin-6-yl}methyl)hydrazine carboxylate

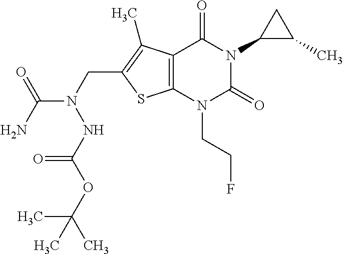 [SMILES: Cc1c(CN(NC(=O)OC(C)(C)C)C(N)=O)sc2c1c(=O)n([C@H]1C[C@@H]1C)c(=O)n2CCF]

To a solution of 375 mg (0.791 mmol, 90% purity) of the compound from Ex. 504A in 25 ml of isopropanol were added 236 μl (1.76 mmol) of trimethylsilyl isocyanate, and the mixture was stirred at RT for 2 days. Thereafter, the reaction mixture was concentrated. The remaining residue was taken up in ethyl acetate and washed successively with saturated solutions of sodium hydrogencarbonate and sodium chloride. After drying over anhydrous magnesium sulfate, the mixture was filtered and concentrated, and the residue was dried under high vacuum. 465 mg (100% of theory, 80% purity) of the title compound were obtained.

LC/MS (Method 1, ESIpos): Rₜ=1.51 min, m/z=470.19 [M+H]⁺.

### Example 519A

### tert-Butyl 2-carbamoyl-2-({1-(2,2-difluoroethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

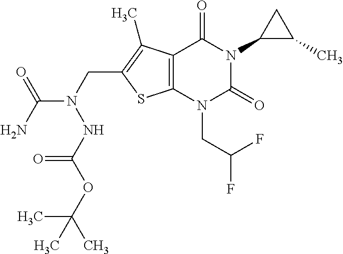 [SMILES: Cc1c(CN(NC(=O)OC(C)(C)C)C(N)=O)sc2c1c(=O)n([C@H]1C[C@@H]1C)c(=O)n2CC(F)F]

Analogously to the method described in Ex. 518A, 200 mg (0.324 mmol, 72% purity) of the compound from Ex. 505A and 121 μl (0.90 mmol) of trimethylsilyl isocyanate were used to obtain 275 mg (quant., 65% purity) of the title compound.

LC/MS (Method 1, ESIneg): Rₜ=1.60 min, m/z=486.16 [M−H].

### Example 520A

### tert-Butyl 2-carbamoyl-2-({5-methyl-3-[(1S,2S)-2-methylcyclopropyl]-2,4-dioxo-1-(2,2,2-trifluoroethyl)-1,2,3,4-tetrahydrothieno[2,3-d]pyrimidin-6-yl}methyl)hydrazinecarboxylate

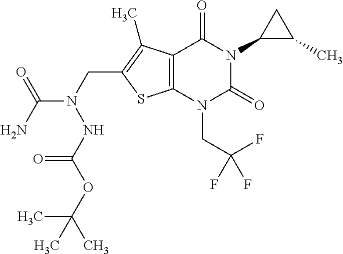 [SMILES: Cc1c(CN(NC(=O)OC(C)(C)C)C(N)=O)sc2c1c(=O)n([C@H]1C[C@@H]1C)c(=O)n2CC(F)(F)F]

Analogously to the method described in Ex. 518A, 165 mg (0.311 mmol, 87% purity) of the compound from Ex. 506A and 96 μl (0.714 mmol) of trimethylsilyl isocyanate were used to obtain 250 mg (quant., 71% purity) of the title compound. The reaction time here was about 16 h.

LC/MS (Method 1, ESIneg): Rₜ=1.73 min, m/z=504.15 [M−H]⁻.

### Example 521A

### tert-Butyl 2-carbamoyl-2-({1-(3-fluoropropyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

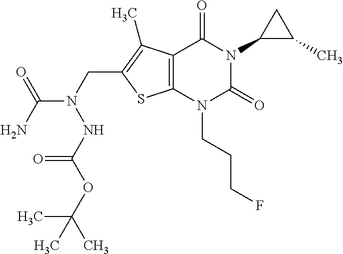 [SMILES: Cc1c(CN(NC(=O)OC(C)(C)C)C(N)=O)sc2c1c(=O)n([C@H]1C[C@@H]1C)c(=O)n2CCCF]

To a solution of 354 mg (0.699 mmol, 87% purity) of the compound from Ex. 507A in 22 ml of isopropanol were added 188 μl (1.40 mmol) of trimethylsilyl isocyanate, and the mixture was stirred at RT for about 18 h. Thereafter, the reaction mixture was concentrated to about half the original volume. In the course of this, a portion of the product precipitated out, which was filtered off with suction, washed with a little pentane and dried under reduced pressure. The mother liquor was fully concentrated and further product was isolated therefrom by means of preparative HPLC (Method 13). The product fraction was concentrated by evaporation and the product was dried under high vacuum. Combination with the solids isolated beforehand gave 285 mg (84% of theory) of the title compound.

¹H-NMR (500 MHz, DMSO-d₆, δ/ppm): 8.90 (br. s, 1H), 6.17 (br. s, 2H), 5.04-4.08 (m, 2H), 4.53 (dt, 2H), 4.02-3.87 (m, 2H), 2.32 (s, 3H), 2.25 (dt, 1H), 2.12-1.97 (m, 2H), 1.38 (br. s, 9H), 1.15 (d, 3H), 1.01-0.91 (m, 1H), 0.88-0.75 (m, 2H).

LC/MS (Method 1, ESIpos): Rₜ=1.57 min, m/z=484.20 [M+H]⁺.

### Example 522A

### tert-Butyl 2-carbamoyl-2-({5-methyl-3-[(1S,2S)-2-methylcyclopropyl]-2,4-dioxo-1-(3,3,3-trifluoropropyl)-1,2,3,4-tetrahydrothieno[2,3-d]pyrimidin-6-yl}methyl)hydrazinecarboxylate

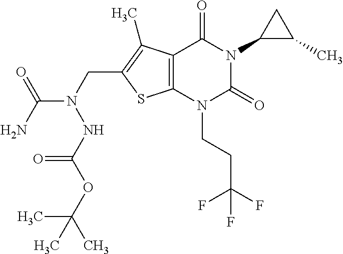 [SMILES: Cc1c(CN(NC(=O)OC(C)(C)C)C(N)=O)sc2c1c(=O)n([C@H]1C[C@@H]1C)c(=O)n2CCC(F)(F)F]

Analogously to the method described in Ex. 473A, 321 mg (0.586 mmol, 87% purity) of the compound from Ex. 508A and 118 μl (0.879 mmol) of trimethylsilyl isocyanate were used to prepare 316 mg (98% of theory, 95% purity) of the title compound.

¹H-NMR (500 MHz, DMSO-d₆, δ/ppm): 8.91 (br. s, 1H), 6.19 (br. s, 2H), 5.01-4.17 (m, 2H), 4.14-4.02 (m, 2H), 2.79-2.66 (m, 2H), 2.32 (s, 3H), 2.26 (dt, 1H), 1.38 (br. s, 9H), 1.15 (d, 3H), 1.01-0.91 (m, 1H), 0.88-0.76 (m, 2H).

LC/MS (Method 1, ESIneg): Rₜ=1.76 min, m/z=518.17 [M−H]⁻.

### Example 523A

### tert-Butyl 2-carbamoyl-2-({5-methyl-3-[(1S,2S)-2-methylcyclopropyl]-2,4-dioxo-1-(4,4,4-trifluorobutyl)-1,2,3,4-tetrahydrothieno[2,3-d]pyrimidin-6-yl}methyl)hydrazinecarboxylate

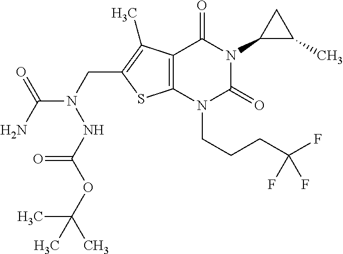 [SMILES: Cc1c(CN(NC(=O)OC(C)(C)C)C(N)=O)sc2c1c(=O)n([C@H]1C[C@@H]1C)c(=O)n2CCCC(F)(F)F]

To a solution of 380 mg (0.713 mmol, 92% purity) of the compound from Ex. 509A in 14 ml of isopropanol were added 191 μl (1.43 mmol) of trimethylsilyl isocyanate, and the mixture was stirred at RT for about 18 h. Thereafter, 100 ml of water were added to the reaction mixture and extraction was effected twice with ethyl acetate. The organic extract was washed with saturated sodium chloride solution, dried over anhydrous magnesium sulfate, filtered and concentrated. The crude product thus obtained was purified by means of MPLC (Biotage Isolera One, SNAP Ultra cartridge, 10 g of silica gel, cyclohexane/ethyl acetate gradient). After concentration of the product fraction and drying under high vacuum, 325 mg (74% of theory, 87% purity) of the title compound were obtained.

¹H-NMR (500 MHz, DMSO-d₆, δ/ppm): 8.89 (br. s, 1H), 6.18 (br. s, 2H), 5.05-4.14 (m, 2H), 3.99-3.84 (m, 2H), 2.46-2.35 (m, 2H), 2.32 (s, 3H), 2.28-2.21 (m, 1H), 1.88 (quin, 2H), 1.38 (br. s, 9H), 1.15 (d, 3H), 1.01-0.91 (m, 1H), 0.88-0.75 (m, 2H).

LC/MS (Method 1, ESIpos): Rₜ=1.80 min, m/z=534.20 [M+H]⁺.

### Example 524A

### tert-Butyl 2-carbamoyl-2-({1-(2-methoxyethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

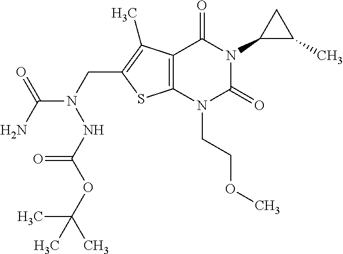 [SMILES: COCCn1c(=O)n([C@H]2C[C@@H]2C)c(=O)c2c(C)c(CN(NC(=O)OC(C)(C)C)C(N)=O)sc21]

Analogously to the method described in Ex. 521A, 308 mg (0.688 mmol, 98% purity) of the compound from Ex. 510A and 323 μl (2.41 mmol) of trimethylsilyl isocyanate were used to prepare 243 mg (71% of theory, 97% purity) of the title compound.

¹H-NMR (500 MHz, DMSO-d₆, δ/ppm): 8.91 (br. s, 1H), 6.17 (br. s, 2H), 5.06-4.14 (m, 2H), 4.06-3.91 (m, 2H), 3.61 (t, 2H), 3.24 (s, 3H), 2.31 (s, 3H), 2.25 (dt, 1H), 1.38 (br. s, 9H), 1.15 (d, 3H), 1.01-0.90 (m, 1H), 0.88-0.76 (m, 2H).

LC/MS (Method 1, ESIpos): Rₜ=1.50 min, m/z=482.21 [M+H]⁺.

### Example 525A

### tert-Butyl 2-carbamoyl-2-({1-[(2,2-difluorocyclopropyl)methyl]-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate (diastereomer mixture)

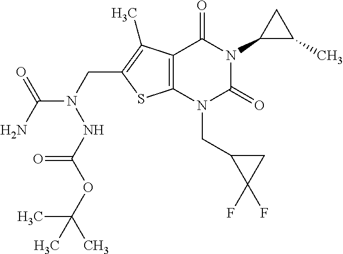 [SMILES: Cc1c(CN(NC(=O)OC(C)(C)C)C(N)=O)sc2c1c(=O)n([C@H]1C[C@@H]1C)c(=O)n2CC1CC1(F)F]

Analogously to the method described in Ex. 518A, 345 mg (0.638 mmol, 87% purity) of the compound from Ex. 511A and 171 μl (1.28 mmol) of trimethylsilyl isocyanate were used to prepare 390 mg (quant., 84% purity) of the title compound.

LC/MS (Method 1, ESIpos): Rₜ=1.71 min, m/z=514.19 [M+H]⁺.

### Example 526A

### tert-Butyl 2-carbamoyl-2-({1-(cyclobutylmethyl)-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

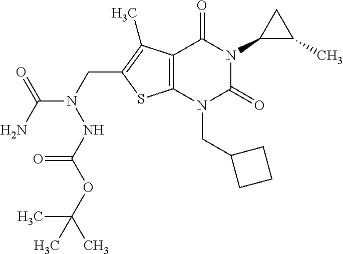 [SMILES: Cc1c(CN(NC(=O)OC(C)(C)C)C(N)=O)sc2c1c(=O)n([C@H]1C[C@@H]1C)c(=O)n2CC1CCC1]

Analogously to the method described in Ex. 523A, 270 mg (0.554 mmol, 92% purity) of the compound from Ex. 512A and 149 μl (1.11 mmol) of trimethylsilyl isocyanate were used to prepare 281 mg (92% of theory, 89% purity) of the title compound.

¹H-NMR (500 MHz, DMSO-d₆, δ/ppm): 8.90 (br. s, 1H), 6.17 (br. s, 2H), 4.97-4.19 (m, 2H), 3.96-3.83 (m, 2H), 2.82-2.69 (m, 1H), 2.31 (s, 3H), 2.25 (dt, 1H), 2.02-1.90 (m, 2H), 1.87-1.74 (m, 4H), 1.38 (br. s, 9H), 1.15 (d, 3H), 0.98-0.88 (m, 1H), 0.87-0.73 (m, 2H).

LC/MS (Method 1, ESIpos): Rₜ=1.84 min, m/z=492.23 [M+H]⁺.

### Example 527A

### tert-Butyl 2-carbamoyl-2-({1-[(3,3-difluorocyclobutyl)methyl]-5-methyl-3-[(1S,2S)-2-methylcyclopropyl]-2,4-dioxo-1,2,3,4-tetrahydrothieno[2,3-d]pyrimidin-6-yl}methyl)hydrazinecarboxylate

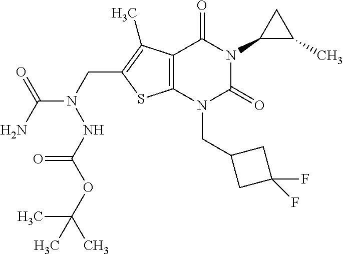 [SMILES: Cc1c(CN(NC(=O)OC(C)(C)C)C(N)=O)sc2c1c(=O)n([C@H]1C[C@@H]1C)c(=O)n2CC1CC(F)(F)C1]

Analogously to the method described in Ex. 521A, 325 mg (0.590 mmol, 87% purity) of the compound from Ex. 513A and 277 μl (2.07 mmol) of trimethylsilyl isocyanate were used to prepare 289 mg (92% of theory) of the title compound.

¹H-NMR (500 MHz, DMSO-d₆, δ/ppm): 8.90 (br. s, 1H), 6.18 (br. s, 2H), 4.95-4.21 (m, 2H), 4.09-3.96 (m, 2H), 2.72-2.57 (m, 3H), 2.56-2.45 (m, 2H, partially concealed by DMSO signal), 2.31 (s, 3H), 2.25 (dt, 1H), 1.38 (br. s, 9H), 1.15 (d, 3H), 1.00-0.90 (m, 1H), 0.87-0.74 (m, 2H).

LC/MS (Method 1, ESIpos): Rₜ=1.77 min, m/z=528.21 [M+H]⁺.

### Example 528A

### tert-Butyl 2-carbamoyl-2-({5-methyl-3-[(1S,2S)-2-methylcyclopropyl]-2,4-dioxo-1-[(2R)-tetrahydrofuran-2-ylmethyl]-1,2,3,4-tetrahydrothieno[2,3-d]pyrimidin-6-yl}methyl)hydrazinecarboxylate

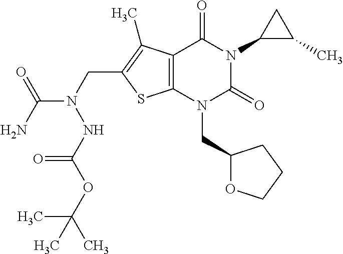 [SMILES: Cc1c(CN(NC(=O)OC(C)(C)C)C(N)=O)sc2c1c(=O)n([C@H]1C[C@@H]1C)c(=O)n2C[C@H]1CCCO1]

Analogously to the method described in Ex. 518A, 105 mg (0.158 mmol, 70% purity) of the compound from Ex. 514A and 61 μl (0.452 mmol) of trimethylsilyl isocyanate were used to obtain 172 mg (quant., 65% purity) of the title compound.

LC/MS (Method 1, ESIneg): Rₜ=1.61 min, m/z=506.21 [M−H].

### WORKING EXAMPLES

### Example 1

### 3-Cyclopropyl-1,5-dimethyl-6-[(2-oxoimidazolidin-1-yl)methyl]thieno[2,3-d]pyrimidine-2,4(1H,3H)-dione

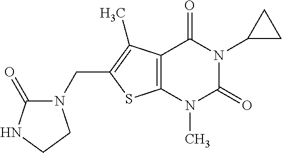 [SMILES: Cc1c(CN2CCNC2=O)sc2c1c(=O)n(C1CC1)c(=O)n2C]

To a solution of 290 mg (0.752 mmol, 80% purity) of the compound from Ex. 100A and 157 μl (1.13 mmol) of triethylamine in 8 ml of THF were added 146 mg (0.903 mmol) of N,N′-carbonyldiimidazole (CDI), and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was prepurified by means of preparative HPLC (Method 11). Concentration of the product fractions was followed by a second normal-phase chromatography operation (MPLC, Biotage Isolera One, SNAP KP-Sil cartridge, 10 g of silica, eluent: ethyl acetate). The product fractions were combined, concentrated and dried in high vacuum. 37 mg (14% of theory) of the title compound were obtained.

¹H-NMR (400 MHz, DMSO-d₆, δ/ppm): 6.51 (s, 1H), 4.34 (s, 2H), 3.37 (s, 3H), 3.27-3.16 (m, 4H), 2.58 (tt, 1H), 2.38 (s, 3H), 1.05-0.95 (m, 2H), 0.71-0.63 (m, 2H).

LC/MS (Method 1, ESIpos): Rₜ=1.05 min, m/z=335.12 [M+H]⁺.

### Example 2

### 3-Cyclopropyl-1,5-dimethyl-6-[(5-oxo-4,5-dihydro-1H-1,2,4-triazol-1-yl)methyl]thieno[2,3-d]pyrimidine-2,4(1H,3H)-dione

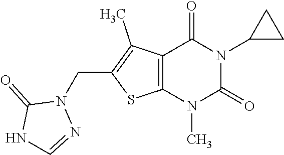 [SMILES: Cc1c(Cn2nc[nH]c2=O)sc2c1c(=O)n(C1CC1)c(=O)n2C]

130 mg (0.307 mmol) of the compound from Ex. 244A were dissolved in a mixture of 10 ml each of methanol and trimethyl orthoformate, and 767 μl (3.07 mmol) of a 4 M solution of hydrogen chloride in dioxane were added at RT. After 2 days, the reaction mixture was concentrated and the residue was taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 50 mg (48% of theory) of the title compound were obtained.

¹H-NMR (400 MHz, DMSO-d₆, δ/ppm): 11.58 (br. s, 1H), 7.83 (s, 1H), 4.93 (s, 2H), 3.36 (s, 3H), 2.61-2.55 (m, 1H), 2.44 (s, 3H), 1.09-0.89 (m, 2H), 0.74-0.58 (m, 2H).

LC/MS (Method 1, ESIpos): Rₜ=0.95 min, m/z=334.10 [M+H]⁺.

### Example 3

### 1-Butyl-3-cyclopropyl-5-methyl-6-[(2-oxoimidazolidin-1-yl)methyl]thieno[2,3-d]pyrimidine-2,4(1H,3H)-dione

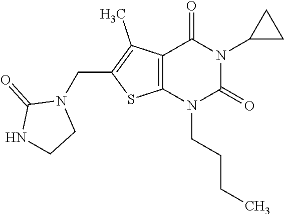 [SMILES: CCCCn1c(=O)n(C2CC2)c(=O)c2c(C)c(CN3CCNC3=O)sc21]

To a solution of 250 mg (0.642 mmol, 90% purity) of the compound from Ex. 101A and 134 μl (0.963 mmol) of triethylamine in 7 ml of THF were added 125 mg (0.770 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 132 mg (54% of theory) of the title compound were obtained.

¹H-NMR (400 MHz, DMSO-d₆, δ/ppm): 6.51 (s, 1H), 4.34 (s, 2H), 3.81 (t, 2H), 3.27-3.16 (m, 4H), 2.63-2.55 (m, 1H), 2.38 (s, 3H), 1.63 (quin, 2H), 1.33 (sext, 2H), 1.05-0.96 (m, 2H), 0.91 (t, 3H), 0.72-0.62 (m, 2H).

LC/MS (Method 1, ESIpos): Rₜ=1.46 min, m/z=377.16 [M+H]⁺.

### Example 4

### 1-Butyl-3-cyclopropyl-5-methyl-6-[(5-oxo-4,5-dihydro-H-1,2,4-triazol-1-yl)methyl]thieno[2,3-d]pyrimidine-2,4(1H,3H)-dione

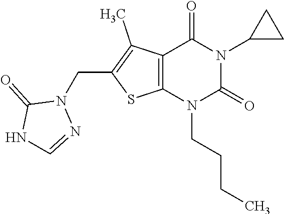 [SMILES: CCCCn1c(=O)n(C2CC2)c(=O)c2c(C)c(Cn3nc[nH]c3=O)sc21]

Analogously to the method described in Ex. 2, 375 mg (0.685 mmol, 85% purity) of the compound from Ex. 245A were used to prepare 177 mg (68% of theory) of the title compound.

¹H-NMR (400 MHz, DMSO-d₆, δ/ppm): 11.58 (br. s, 1H), 7.83 (s, 1H), 4.92 (s, 2H), 3.80 (br. t, 2H), 2.62-2.56 (m, 1H), 2.43 (s, 3H), 1.62 (quin, 2H), 1.32 (sext, 2H), 1.05-0.94 (m, 2H), 0.90 (t, 3H), 0.71-0.61 (m, 2H).

LC/MS (Method 1, ESIpos): Rₜ=1.37 min, m/z=376.14 [M+H]⁺.

### Example 5

### 3-Cyclopropyl-1-(3-fluoropropyl)-5-methyl-6-[(2-oxoimidazolidin-1-yl)methyl]thieno[2,3-d]pyrimidine-2,4(1H,3H)-dione

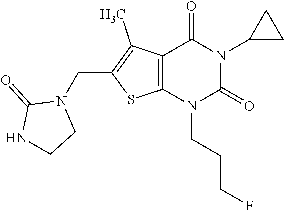 [SMILES: Cc1c(CN2CCNC2=O)sc2c1c(=O)n(C1CC1)c(=O)n2CCCF]

225 mg (0.451 mmol) of the compound from Ex. 102A were dissolved in 20 ml of dioxane, and 113 mg (0.676 mmol) of CDI were added. The mixture was stirred at RT for 19 h. The reaction solution was then concentrated on a rotary evaporator. The residue was dissolved in 3 ml of DMSO and this solution was purified by means of preparative HPLC (Method 14). Combination of the product fractions and freeze-drying gave 102 mg (58% of theory) of the title compound.

¹H-NMR (400 MHz, DMSO-d₆, δ/ppm): 6.54 (s, 1H), 4.58 (t, 1H), 4.46 (t, 1H), 4.34 (s, 2H), 3.93 (t, 2H), 3.27-3.16 (m, 4H), 2.62-2.54 (m, 1H), 2.38 (s, 3H), 2.11-1.96 (m, 2H), 1.03-0.96 (m, 2H), 0.70-0.63 (m, 2H).

LC/MS (Method 4, ESIpos): Rₜ=0.84 min, m/z=381 [M+H]⁺.

### Example 6

### 3-Cyclopropyl-1-(3-fluoropropyl)-5-methyl-6-[(2-thioxoimidazolidin-1-yl)methyl]thieno[2,3-d]pyrimidine-2,4(1H,3H)-dione

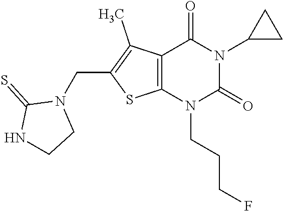 [SMILES: Cc1c(CN2CCNC2=S)sc2c1c(=O)n(C1CC1)c(=O)n2CCCF]

120 mg (0.24 mmol) of the compound from Ex. 102A were dissolved in 15 ml of dioxane, and 68 mg (0.361 mmol) of 1,1′-thiocarbonyldiimidazole were added. The mixture was stirred at RT for 19 h. The reaction solution was concentrated on a rotary evaporator. The residue was dissolved in 3 ml of DMSO and this solution was purified by means of preparative HPLC (Method 14). Combination of the product fractions and freeze-drying gave 40 mg (40% of theory) of the title compound.

¹H-NMR (400 MHz, DMSO-d₆, δ/ppm): 8.35 (s, 1H), 4.82 (s, 2H), 4.58 (t, 1H), 4.46 (t, 1H), 3.93 (t, 2H), 3.56-3.48 (m, 2H), 3.43-3.36 (m, 2H), 2.62-2.55 (m, 1H), 2.42 (s, 3H), 2.11-1.96 (m, 2H), 1.03-0.96 (m, 2H), 0.70-0.63 (m, 2H).

LC/MS (Method 4, ESIpos): Rₜ=0.94 min, m/z=397 [M+H]⁺.

### Example 7

### [1-{[3-Cyclopropyl-1-(3-fluoropropyl)-5-methyl-2,4-dioxo-1,2,3,4-tetrahydrothieno[2,3-d]pyrimidin-6-yl]methyl}imidazolidin-2-ylidene]cyanamide

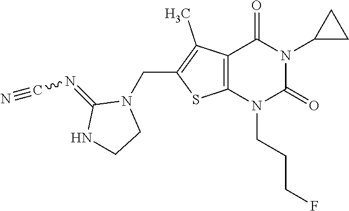 [SMILES: Cc1c(CN2CCNC2=NC#N)sc2c1c(=O)n(C1CC1)c(=O)n2CCCF]

120 mg (0.24 mmol) of the compound from Ex. 102A were dissolved in 5 ml of DMF, and 56 mg (0.361 mmol) of dimethyl N-cyanodithioiminocarbonate and 67 mg (0.481 mmol) of potassium carbonate were added. The mixture was stirred at 80° C. for 19 h. 30 ml of ethyl acetate were then added. The mixture was washed with saturated aqueous sodium hydrogencarbonate solution and water. The organic phase was dried over anhydrous sodium sulfate, filtered and concentrated. The residue was dissolved in 3 ml of DMSO and this solution was purified by means of preparative HPLC (Method 14). Combination of the product fractions and freeze-drying gave 40 mg (40% of theory) of the title compound.

¹H-NMR (400 MHz, DMSO-d₆, δ/ppm): 8.08 (s, 1H), 4.58 (t, 1H), 4.50-4.44 (m, 3H), 3.94 (t, 2H), 3.48-3.37 (m, 4H), 2.62-2.55 (m, 1H), 2.39 (s, 3H), 2.12-1.97 (m, 2H), 1.03-0.96 (m, 2H), 0.70-0.63 (m, 2H).

LC/MS (Method 4, ESIpos): Rₜ=0.90 min, m/z=405 [M+H]⁺.

### Example 8

### 3-Cyclopropyl-6-[(2,3-dioxopiperazin-1-yl)methyl]-1-(3-fluoropropyl)-5-methylthieno[2,3-d]pyrimidine-2,4(1H,3H)-dione

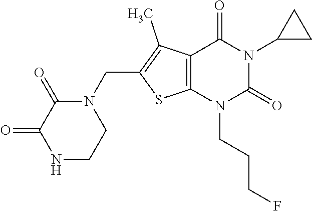 [SMILES: Cc1c(CN2CCNC(=O)C2=O)sc2c1c(=O)n(C1CC1)c(=O)n2CCCF]

120 mg (0.24 mmol) of the compound from Ex. 102A were dissolved in 5 ml of ethanol, and 335 mg (2.4 mmol) of diethyl oxalate were added. The mixture was stirred at 80° C. for 19 h. Thereafter, the reaction solution was concentrated on a rotary evaporator and the residue was dissolved in 30 ml of dichloromethane. The mixture was washed with water and saturated sodium hydrogencarbonate solution. The organic phase was dried over sodium sulfate, filtered and concentrated. The residue obtained was dissolved in 3 ml of DMSO and this solution was purified by means of preparative HPLC (Method 14). Combination of the product fractions and freeze-drying gave 5 mg (5% of theory) of the title compound.

¹H-NMR (400 MHz, DMSO-d₆, δ/ppm): 8.64 (br. s, 1H), 4.68 (s, 2H), 4.58 (t, 1H), 4.46 (t, 1H), 3.93 (t, 2H), 3.50-3.44 (m, 2H), 3.32-3.27 (m, 2H), 2.62-2.54 (m, 1H), 2.42 (s, 3H), 2.12-1.95 (m, 2H), 1.04-0.96 (m, 2H), 0.70-0.63 (m, 2H).

LC/MS (Method 4, ESIneg): Rₜ=0.76 min, m/z=453 [M−H+HCO₂H]⁻.

### Example 9

### 3-Cyclopropyl-5-methyl-6-[(2-oxoimidazolidin-1-yl)methyl]-1-(3,3,3-trifluoropropyl)thieno[2,3-d]pyrimidine-2,4(1H,3H)-dione

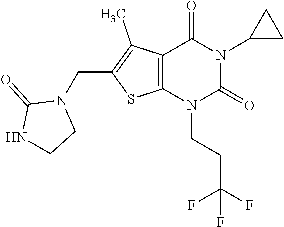 [SMILES: Cc1c(CN2CCNC2=O)sc2c1c(=O)n(C1CC1)c(=O)n2CCC(F)(F)F]

Analogously to Ex. 5, 205 mg (0.42 mmol) of the compound from Ex. 103A in 20 ml of dioxane were reacted with 105 mg (0.63 mmol) of CDI. 116 mg (65% of theory) of the title compound were obtained.

¹H-NMR (400 MHz, DMSO-d₆, δ/ppm): 6.55 (s, 1H), 4.35 (s, 2H), 4.05 (t, 2H), 3.28-3.16 (m, 4H), 2.80-2.68 (m, 2H), 2.59 (tt, 1H), 2.38 (s, 3H), 1.05-0.96 (m, 2H), 0.70-0.63 (m, 2H).

LC/MS (Method 4, ESIpos): Rₜ=0.95 min, m/z=417 [M+H]⁺.

### Example 10

### 3-Cyclopropyl-5-methyl-6-[(2-thioxoimidazolidin-1-yl)methyl]-1-(3,3,3-trifluoropropyl)thieno[2,3-d]pyrimidine-2,4(1H,3H)-dione

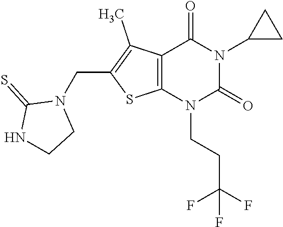 [SMILES: Cc1c(CN2CCNC2=S)sc2c1c(=O)n(C1CC1)c(=O)n2CCC(F)(F)F]

Analogously to Ex. 6, 130 mg (0.266 mmol) of the compound from Ex. 103A in 15 ml of dioxane were reacted with 75 mg (0.4 mmol) of 1,1′-thiocarbonyldiimidazole. 56 mg (48% of theory) of the title compound were obtained.

¹H-NMR (400 MHz, DMSO-d₆, δ/ppm): 8.36 (s, 1H), 4.83 (s, 2H), 4.05 (t, 2H), 3.57-3.47 (m, 2H), 3.45-3.36 (m, 2H), 2.82-2.68 (m, 2H), 2.64-2.56 (m, 1H), 2.42 (s, 3H), 1.05-0.96 (m, 2H), 0.71-0.63 (m, 2H).

LC/MS (Method 4, ESIpos): Rₜ=1.05 min, m/z=433 [M+H]⁺.

### Example 11

### [1-{[3-Cyclopropyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

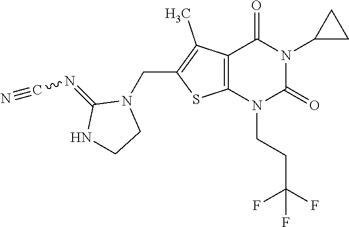 [SMILES: Cc1c(CN2CCNC2=NC#N)sc2c1c(=O)n(C1CC1)c(=O)n2CCC(F)(F)F]

130 mg (0.266 mmol) of the compound from Ex. 103A were dissolved in 5 ml of DMF, and 62 mg (0.4 mmol) of dimethyl N-cyanodithioiminocarbonate and 74 mg (0.649 mmol) of potassium carbonate were added. The mixture was stirred at 80° C. for 19 h. 30 ml of ethyl acetate were then added. The mixture was washed with saturated aqueous sodium hydrogencarbonate solution and water. The organic phase was dried over anhydrous sodium sulfate, filtered and concentrated. The residue was dissolved in 3 ml of DMSO and this solution was purified by means of preparative HPLC (Method 14). Combination of the product fractions and freeze-drying gave 46 mg (38% of theory) of the title compound.

¹H-NMR (400 MHz, DMSO-d₆, δ/ppm): 8.10 (s, 1H), 4.48 (s, 2H), 4.06 (t, 2H), 3.49-3.37 (m, 4H), 2.81-2.69 (m, 2H), 2.59 (tt, 1H), 2.39 (s, 3H), 1.05-0.97 (m, 2H), 0.70-0.63 (m, 2H).

LC/MS (Method 4, ESIpos): Rₜ=1.0 min, m/z=441 [M+H]⁺.

### Example 12

### Methyl [1-{[3-cyclopropyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

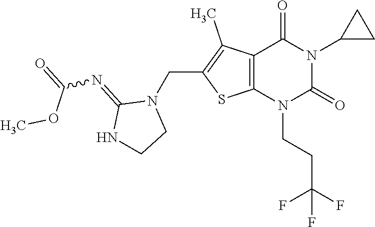 [SMILES: COC(=O)N=C1NCCN1Cc1sc2c(c1C)c(=O)n(C1CC1)c(=O)n2CCC(F)(F)F]

310 mg (0.556 mmol, 70% purity) of the compound from Ex. 103A and 155 μl (1.11 mmol) of triethylamine were dissolved in 10 ml of dichloromethane, and a solution of 173 mg (1.11 mmol) of methyl (dichloromethylene)carbamate in 5 ml of dichloromethane was added dropwise. After the reaction mixture had been stirred at RT for about 18 h, it was concentrated to dryness. The residue was stirred with a little acetonitrile at RT. The solids were filtered off with suction and discarded. The filtrate was concentrated, and the residue was prepurified by means of preparative HPLC (method 11). The product fraction obtained was repurified for a second time by means of preparative HPLC (column: Kinetex C18, 5 μm, 100 mm×30 mm; eluent A: water+0.07% formic acid, eluent B: acetonitrile; gradient: 0.0-2.0 min 10% B, 2.2 min 20% B, 7.0 min 60% B, 7.5-9.0 min 92% B; flow rate: 70 ml/min; temperature: 25° C.; detection: 210 nm). Concentration of the product fraction and drying of the residue under high vacuum gave 46 mg (17% of theory) of the title compound.

¹H-NMR (400 MHz, DMSO-d₆, δ/ppm): 8.02 (s, 1H), 4.56 (s, 2H), 4.04 (t, 2H), 3.53 (s, 3H), 3.49-3.41 (m, 2H), 3.36-3.28 (m, 2H, partially concealed by water signal), 2.81-2.65 (m, 2H), 2.63-2.56 (m, 1H), 2.41 (s, 3H), 1.05-0.96 (m, 2H), 0.74-0.61 (m, 2H).

LC/MS (Method 1, ESIpos): Rₜ=1.39 min, m/z=474.14 [M+H]⁺.

### Example 13

### 3-Cyclopropyl-6-[(2,3-dioxopiperazin-1-yl)methyl]-5-methyl-1-(3,3,3-trifluoropropyl)thieno[2,3-d]pyrimidine-2,4(1H,3H)-dione

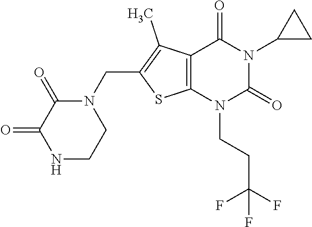 [SMILES: Cc1c(CN2CCNC(=O)C2=O)sc2c1c(=O)n(C1CC1)c(=O)n2CCC(F)(F)F]

130 mg (0.266 mmol) of the compound from Example 103A were dissolved in 5 ml of ethanol, and 393 mg (2.66 mmol) of diethyl oxalate were added. The mixture was stirred at 80° C. for 19 h. Thereafter, the reaction solution was concentrated on a rotary evaporator and the residue was dissolved in 30 ml of dichloromethane. The mixture was washed with water and saturated sodium hydrogencarbonate solution. The organic phase was dried over sodium sulfate, filtered and concentrated. The residue obtained was dissolved in 3 ml of DMSO and this solution was purified by means of preparative HPLC (Method 14). Combination of the product fractions and freeze-drying gave 34 mg (27% of theory) of the title compound.

¹H-NMR (400 MHz, DMSO-d₆, δ/ppm): 8.64 (br. s, 1H), 4.69 (s, 2H), 4.05 (t, 2H), 3.51-3.45 (m, 2H), 3.32-3.27 (m, 2H), 2.81-2.68 (m, 2H), 2.63-2.55 (m, 1H), 2.42 (s, 3H), 1.05-0.97 (m, 2H), 0.70-0.63 (m, 2H).

LC/MS (Method 4, ESIneg): Rₜ=0.86 min, m/z=489 [M−H+HCO₂H]⁻.

### Example 14

### 3-Cyclopropyl-5-methyl-6-[(2-oxo-2,3-dihydro-1H-imidazol-1-yl)methyl]-1-(3,3,3-trifluoropropyl)thieno[2,3-d]pyrimidine-2,4(1H,3H)-dione

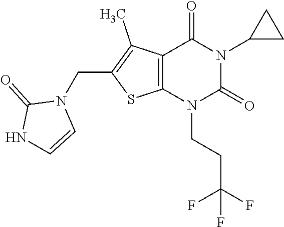 [SMILES: Cc1c(Cn2cc[nH]c2=O)sc2c1c(=O)n(C1CC1)c(=O)n2CCC(F)(F)F]

To a solution of 629 mg (0.93 mmol, 71% purity) of the compound from Ex. 153A in a mixture of 1.9 ml of water and 10 ml of methanol were added 1.9 ml (0.92 mmol) of 0.5 M hydrochloric acid. After the reaction mixture had been stirred at RT for 40 h, it was separated into its components directly by means of preparative HPLC (Method 13). Concentration and drying of the product fraction under high vacuum gave 167 mg (43% of theory) of the title compound.

¹H-NMR (400 MHz, DMSO-d₆, δ/ppm): 9.99 (s, 1H), 6.43 (dd, 1H), 6.34 (dd, 1H), 4.79 (s, 2H), 4.03 (dd, 2H), 2.78-2.65 (m, 2H), 2.61-2.58 (m, 1H), 2.45 (s, 3H), 1.03-0.98 (m, 2H), 0.68-0.64 (m, 2H).

LC/MS (Method 1, ESIneg): Rₜ=1.31 min, m/z=413 [M−H]⁻.

### Example 15

### 3-Cyclopropyl-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

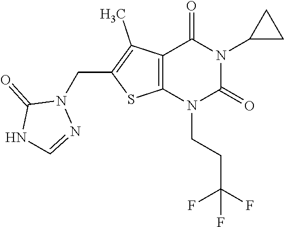 [SMILES: Cc1c(Cn2nc[nH]c2=O)sc2c1c(=O)n(C1CC1)c(=O)n2CCC(F)(F)F]

181 mg (0.301 mmol, 84% purity) of the compound from Ex. 246A were dissolved in a mixture of 7.5 ml each of methanol and trimethyl orthoformate, and 752 μl (3.01 mmol) of a 4 M solution of hydrogen chloride in dioxane were added at RT. After 2 days, a further 371 μl (1.50 mmol) of the 4 M solution of hydrogen chloride in dioxane were added. After a further day, water was added to the reaction mixture, which was extracted with ethyl acetate. The organic extract was concentrated and the residue obtained was purified by means of preparative HPLC (Method 11). After concentration of the product fractions and drying under high vacuum, 99 mg (79% of theory) of the title compound were obtained.

¹H-NMR (500 MHz, DMSO-d₆, δ/ppm): 11.59 (br. s, 1H), 7.83 (s, 1H), 4.94 (s, 2H), 4.04 (t, 2H), 2.79-2.66 (m, 2H), 2.64-2.55 (m, 1H), 2.44 (s, 3H), 1.09-0.92 (m, 2H), 0.78-0.58 (m, 2H).

LC/MS (Method 1, ESIpos): Rₜ=1.29 min, m/z=416.10 [M+H]⁺.

### Example 16

### 3-Cyclopropyl-5-methyl-6-[(2-oxoimidazolidin-1-yl)dideuteromethyl]-1-(3,3,3-trifluoropropyl)thieno[2,3-d]pyrimidine-2,4(1H,3H)-dione

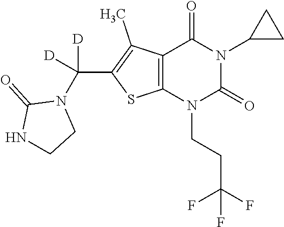 [SMILES: [2H]C([2H])(c1sc2c(c1C)c(=O)n(C1CC1)c(=O)n2CCC(F)(F)F)N1CCNC1=O]

To a solution of 286 mg (3.32 mmol) of imidazolidin-2-one in 8 ml of DMF were added 133 mg (3.32 mmol) of sodium hydride (60% suspension in mineral oil), then the mixture was heated to 60° C. for 5 min and subsequently cooled back down to RT (“Solution 1”). To a solution of 291 mg (0.831 mmol) of the compound from Ex. 292A in 6 ml of dichloromethane in another reaction vessel were added, at 0° C., 289 μl (1.66 mmol) of N,N-diisopropylethylamine and 64 μl (0.872 mmol) of thionyl chloride. After 20 min, Solution 1 was added in portions at 0° C. The reaction mixture was then stirred at RT for 2.5 days. Then water was added and extraction was effected with ethyl acetate. The organic extract was washed with water and saturated sodium chloride solution. After drying over anhydrous magnesium sulfate, the mixture was filtered and concentrated. The residue that remained was first prepurified by means of MPLC (instrument: Biotage Isolera One, SNAP KP-Sil cartridge, 25 g of silica gel, eluent: cyclohexane/ethyl acetate 1:1-dichloromethane/methanol 10:1). The still contaminated product fractions were then repurified by means of preparative HPLC (Method 11). After concentration of the product fractions and drying under high vacuum, 164 mg (47% of theory) of the title compound were obtained.

¹H-NMR (400 MHz, DMSO-d₆, δ/ppm): 6.52 (s, 1H), 4.05 (t, 2H), 3.28-3.16 (m, 4H), 2.81-2.66 (m, 2H), 2.60 (tt, 1H), 2.38 (s, 3H), 1.08-0.93 (m, 2H), 0.74-0.60 (m, 2H).

LC/MS (Method 1, ESIpos): Rₜ=1.38 min, m/z=419.13 [M+H]⁺.

### Example 17

### 1-(Cyclobutylmethyl)-3-cyclopropyl-5-methyl-6-[(2-oxoimidazolidin-1-yl)methyl]thieno[2,3-d]pyrimidine-2,4(1H,3H)-dione

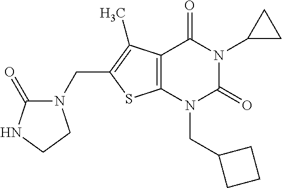 [SMILES: Cc1c(CN2CCNC2=O)sc2c1c(=O)n(C1CC1)c(=O)n2CC1CCC1]

To a solution of 240 mg (0.616 mmol, 93% purity) of the compound from Ex. 104A and 129 μl (0.924 mmol) of triethylamine in 7 ml of THF were added 120 mg (0.739 mmol) of CDI, and the mixture was stirred at RT for about 16 h. Subsequently, the mixture was concentrated to dryness. The remaining residue was taken up in ethyl acetate and washed successively with water and saturated sodium chloride solution. After drying over anhydrous magnesium sulfate, the mixture was filtered and concentrated. The solid residue was purified by means of preparative HPLC (Method 11). The product fractions were combined, concentrated by evaporation and dried under high vacuum. 147 mg (61% of theory) of the title compound were obtained.

¹H-NMR (400 MHz, DMSO-d₆, δ/ppm): 6.51 (s, 1H), 4.33 (s, 2H), 3.88 (d, 2H), 3.28-3.14 (m, 4H), 2.81-2.67 (m, 1H), 2.63-2.55 (m, 1H), 2.37 (s, 3H), 2.05-1.89 (m, 2H), 1.87-1.72 (m, 4H), 1.08-0.92 (m, 2H), 0.75-0.59 (m, 2H).

LC/MS (Method 1, ESIpos): Rₜ=1.50 min, m/z=389.16 [M+H]⁺.

### Example 18

### 1-(Cyclobutylmethyl)-3-cyclopropyl-5-methyl-6-[(5-oxo-4,5-dihydro-1H-1,2,4-triazol-1-yl)methyl]thieno[2,3-d]pyrimidine-2,4(1H,3H)-dione

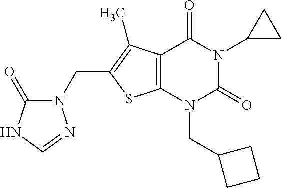 [SMILES: Cc1c(Cn2nc[nH]c2=O)sc2c1c(=O)n(C1CC1)c(=O)n2CC1CCC1]

Analogously to the method described in Ex. 2, 370 mg (0.775 mmol) of the compound from Ex. 247A were used to prepare 193 mg (64% of theory) of the title compound.

¹H-NMR (400 MHz, DMSO-d₆, δ/ppm): 11.58 (s, 1H), 7.83 (s, 1H), 4.91 (s, 2H), 3.87 (d, 2H), 2.80-2.65 (m, 1H), 2.63-2.55 (m, 1H), 2.43 (s, 3H), 2.04-1.88 (m, 2H), 1.87-1.71 (m, 4H), 1.09-0.89 (m, 2H), 0.75-0.56 (m, 2H).

LC/MS (Method 1, ESIpos): Rₜ=1.42 min, m/z=388.14 [M+H]⁺.

### Example 19

### 3-Cyclopropyl-1-[(2,2-difluorocyclopropyl)methyl]-5-methyl-6-[(2-oxoimidazolidin-1-yl)methyl]thieno[2,3-d]pyrimidine-2,4(1H,3H)-dione (racemate)

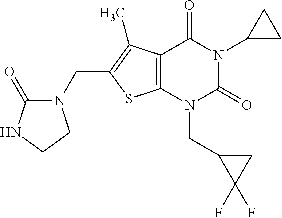 [SMILES: Cc1c(CN2CCNC2=O)sc2c1c(=O)n(C1CC1)c(=O)n2CC1CC1(F)F]

Analogously to the method described in Ex. 3, 495 mg (1.19 mmol, 92% purity) of the compound from Ex. 105A and 230 mg (1.42 mmol) of CDI were used to prepare 254 mg (52% of theory) of the title compound.

¹H-NMR (400 MHz, DMSO-d₆, δ/ppm): 6.52 (s, 1H), 4.35 (s, 2H), 4.13-4.00 (m, 1H), 3.90 (dd, 1H), 3.28-3.16 (m, 4H), 2.60 (tt, 1H), 2.38 (s, 3H), 2.26-2.10 (m, 1H), 1.75-1.62 (m, 1H), 1.51-1.38 (m, 1H), 1.05-0.96 (m, 2H), 0.73-0.64 (m, 2H).

LC/MS (Method 1, ESIpos): Rₜ=1.36 min, m/z=411.13 [M+H]⁺.

### Example 20

### 3-Cyclopropyl-1-[(2,2-difluorocyclopropyl)methyl]-5-methyl-6-[(5-oxo-4,5-dihydro-1H-1,2,4-triazol-1-yl)methyl]thieno[2,3-d]pyrimidine-2,4(1H,3H)-dione (racemate)

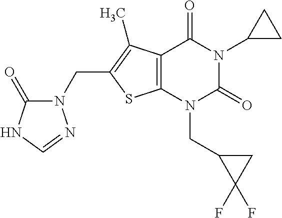 [SMILES: Cc1c(Cn2nc[nH]c2=O)sc2c1c(=O)n(C1CC1)c(=O)n2CC1CC1(F)F]

Analogously to the method described in Ex. 2, 655 mg (1.22 mmol, 93% purity) of the compound from Ex. 248A were used to prepare 380 mg (76% of theory) of the title compound.

¹H-NMR (400 MHz, DMSO-d₆, δ/ppm): 11.59 (s, 1H), 7.83 (s, 1H), 4.93 (s, 2H), 4.13-4.02 (m, 1H), 3.87 (dd, 1H), 2.64-2.56 (m, 1H), 2.44 (s, 3H), 2.24-2.08 (m, 1H), 1.77-1.59 (m, 1H), 1.52-1.36 (m, 1H), 1.07-0.92 (m, 2H), 0.74-0.60 (m, 2H).

LC/MS (Method 2, ESIpos): Rₜ=0.70 min, m/z=410 [M+H]⁺.

### Example 21

### 3-Cyclopropyl-1-[(2,2-difluorocyclopropyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 1)

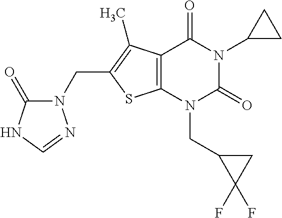 [SMILES: Cc1c(Cn2nc[nH]c2=O)sc2c1c(=O)n(C1CC1)c(=O)n2CC1CC1(F)F]

372 mg of the racemic compound from Ex. 20 were dissolved in a mixture of 5 ml of ethanol and 5 ml of dioxane and, in 20 portions, separated into the enantiomers via preparative HPLC on a chiral phase [column: Daicel Chiralcel, 5 μm, 250 mm×20 mm; eluent: n-heptane/ethanol 1:1; flow rate: 15 ml/min; temperature: 25° C.; detection: 210 nm]. After concentration of the product fractions and drying of the solids under high vacuum, 171 mg (91% of theory) of enantiomer 1 were obtained (98.6% ee, chiral analytical HPLC).

¹H-NMR (400 MHz, DMSO-d₆, δ/ppm): 11.58 (br. s, 1H), 7.82 (s, 1H), 4.93 (s, 2H), 4.13-4.01 (m, 1H), 3.87 (dd, 1H), 2.60 (tt, 1H), 2.44 (s, 3H), 2.24-2.08 (m, 1H), 1.75-1.60 (m, 1H), 1.52-1.37 (m, 1H), 1.07-0.94 (m, 2H), 0.72-0.61 (m, 2H).

Chiral analytical HPLC [column: Phenomenex Cellulose, 3 μm, 50 mm×4.6 mm; eluent: isohexane/ethanol 1:1; flow rate: 1 ml/min; temperature: 25° C.; detection: 220 nm]: Rₜ=1.33 min.

### Example 22

### 3-Cyclopropyl-1-[(2,2-difluorocyclopropyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 2)

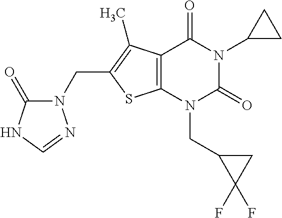 [SMILES: Cc1c(Cn2nc[nH]c2=O)sc2c1c(=O)n(C1CC1)c(=O)n2CC1CC1(F)F]

372 mg of the racemic compound from Ex. 20 were dissolved in a mixture of 5 ml of ethanol and 5 ml of dioxane and, in 20 portions, separated into the enantiomers via preparative HPLC on a chiral phase [column: Daicel Chiralcel, 5 μm, 250 mm×20 mm; eluent: n-heptane/ethanol 1:1; flow rate: 15 ml/min; temperature: 25° C.; detection: 210 nm]. After concentration of the product fractions and drying of the solids under high vacuum, 166 mg (89% of theory) of enantiomer 2 were obtained (99.1% ee, chiral analytical HPLC).

¹H-NMR (400 MHz, DMSO-d₆, δ/ppm): 11.59 (br. s, 1H), 7.82 (s, 1H), 4.93 (s, 2H), 4.15-4.00 (m, 1H), 3.87 (dd, 1H), 2.64-2.56 (m, 1H), 2.44 (s, 3H), 2.23-2.08 (m, 1H), 1.75-1.61 (m, 1H), 1.51-1.38 (m, 1H), 1.05-0.94 (m, 2H), 0.72-0.63 (m, 2H).

Chiral analytical HPLC [column: Phenomenex Cellulose, 3 μm, 50 mm×4.6 mm; eluent: isohexane/ethanol 1:1; flow rate: 1 ml/min; temperature: 25° C.; detection: 220 nm]: Rₜ=1.85 min.

### Example 23

### {3-Cyclopropyl-5-methyl-2,4-dioxo-6-[(5-oxo-4,5-dihydro-1H-1,2,4-triazol-1-yl)methyl]-3,4-dihydrothieno[2,3-d]pyrimidin-1 (2H)-yl}acetonitrile

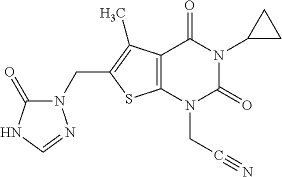 [SMILES: Cc1c(Cn2nc[nH]c2=O)sc2c1c(=O)n(C1CC1)c(=O)n2CC#N]

Analogously to the method described in Ex. 2, 230 mg (0.487 mmol, 95% purity) of the compound from Ex. 249A were used to prepare 110 mg (63% of theory) of the title compound.

¹H-NMR (400 MHz, DMSO-d₆, δ/ppm): 11.61 (br. s, 1H), 7.84 (s, 1H), 5.07 (s, 2H), 4.97 (s, 2H), 2.66-2.57 (m, 1H), 2.45 (s, 3H), 1.10-0.92 (m, 2H), 0.77-0.62 (m, 2H).

LC/MS (Method 1, ESIpos): Rₜ=0.98 min, m/z=359.09 [M+H]⁺.

### Example 24

### 3-Cyclopropyl-1-(2-methoxyethyl)-5-methyl-6-[(2-oxoimidazolidin-1-yl)methyl]thieno[2,3-d]pyrimidine-2,4(1H,3H)-dione

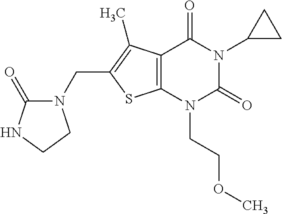 [SMILES: COCCn1c(=O)n(C2CC2)c(=O)c2c(C)c(CN3CCNC3=O)sc21]

138 mg (0.223 mmol) of the compound from Ex. 106A were dissolved in 15 ml of dioxane, and 56 mg (0.335 mmol) of CDI were added. The mixture was stirred at RT for 15 h. The reaction solution was then concentrated on a rotary evaporator. The residue was dissolved in 3 ml of DMSO and this solution was purified by means of preparative HPLC (Method 14). Combination of the product fractions and freeze-drying gave 53 mg (62% of theory) of the title compound.

¹H-NMR (400 MHz, DMSO-d₆, δ/ppm): 6.53 (s, 1H), 4.33 (s, 2H), 3.97 (t, 2H), 3.60 (t, 2H), 3.27-3.15 (m, 7H), 2.59 (tt, 1H), 2.36 (s, 3H), 1.03-0.96 (m, 2H), 0.70-0.63 (m, 2H).

LC/MS (Method 4, ESIpos): Rₜ=0.80 min, m/z=379 [M+H]⁺.

### Example 25

### [1-{[3-Cyclopropyl-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

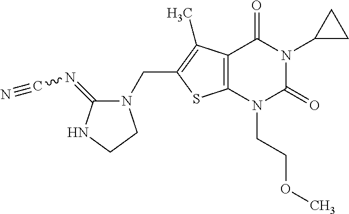 [SMILES: COCCn1c(=O)n(C2CC2)c(=O)c2c(C)c(CN3CCNC3=NC#N)sc21]

145 mg (0.267 mmol) of the compound from Ex. 106A were dissolved in 5 ml of DMF, and 62 mg (0.4 mmol) of dimethyl N-cyanodithioiminocarbonate and 73.9 mg (0.534 mmol) of potassium carbonate were added. The mixture was stirred at 80° C. for 19 h. 30 ml of ethyl acetate were then added. The mixture was washed with saturated aqueous sodium hydrogencarbonate solution and water. The organic phase was dried over anhydrous sodium sulfate, filtered and concentrated. The residue was dissolved in 3 ml of DMSO and this solution was purified by means of preparative HPLC (Method 14). Combination of the product fractions and freeze-drying gave 79 mg (70% of theory) of the title compound.

¹H-NMR (400 MHz, DMSO-d₆, δ/ppm): 8.08 (s, 1H), 4.46 (s, 2H), 3.98 (t, 2H), 3.61 (t, 2H), 3.48-3.36 (m, 4H), 3.23 (s, 3H), 2.63-2.55 (m, 1H), 2.38 (s, 3H), 1.04-0.96 (m, 2H), 0.71-0.63 (m, 2H).

LC/MS (Method 4, ESIpos): Rₜ=0.86 min, m/z=403 [M+H]⁺.

### Example 26

### Methyl [1-{[3-cyclopropyl-1-(2-methoxyethyl)-5-methyl-2,4-dioxo-1,2,3,4-tetrahydrothieno[2,3-d]pyrimidin-6-yl]methyl}imidazolidin-2-ylidene]carbamate

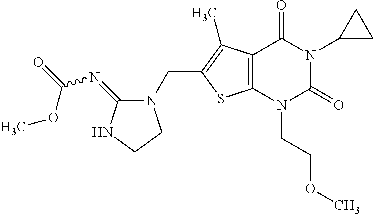 [SMILES: COCCn1c(=O)n(C2CC2)c(=O)c2c(C)c(CN3CCNC3=NC(=O)OC)sc21]

400 mg (0.908 mmol, 80% purity) of the compound from Ex. 106A and 253 μl (1.82 mmol) of triethylamine were dissolved in 15 ml of dichloromethane, and a solution of 283 mg (1.82 mmol) of methyl (dichloromethylene)carbamate in 5 ml of dichloromethane was added dropwise. After the reaction mixture had been stirred at RT for about 18 h, it was concentrated to dryness. The residue was prepurified by means of preparative HPLC (Method 11). Since the product fraction was still contaminated, there followed a second chromatography operation by means of MPLC (Biotage Isolera One, SNAP KP-Sil cartridge, 50 g of silica gel, eluent: ethyl acetate→dichloromethane/methanol 10:1). Concentration of the product fraction and drying of the residue under high vacuum, after stirring with acetonitrile, gave 92 mg (23% of theory) of the title compound.

¹H-NMR (400 MHz, DMSO-d₆, δ/ppm): 8.01 (s, 1H), 4.54 (s, 2H), 3.96 (br. t, 2H), 3.60 (br. t, 2H), 3.53 (s, 3H), 3.49-3.40 (m, 2H), 3.35-3.28 (m, 2H, substantially concealed by water signal), 3.22 (s, 3H), 2.62-2.56 (m, 1H), 2.40 (s, 3H), 1.03-0.97 (m, 2H), 0.70-0.65 (m, 2H).

LC/MS (Method 1, ESIpos): Rₜ=1.05 min, m/z=436.16 [M+H]⁺.

### Example 27

### 3-Cyclopropyl-6-[(2,3-dioxopiperazin-1-yl)methyl]-1-(2-methoxyethyl)-5-methylthieno[2,3-d]pyrimidine-2,4(1H,3H)-dione

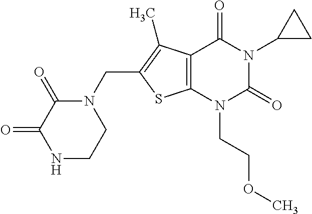 [SMILES: COCCn1c(=O)n(C2CC2)c(=O)c2c(C)c(CN3CCNC(=O)C3=O)sc21]

Analogously to Ex. 8, 145 mg (0.267 mmol) of the compound from Ex. 106A in 5 ml of ethanol were reacted with 395 mg (2.67 mmol) of diethyl oxalate. 28 mg (25% of theory) of the title compound were obtained.

¹H-NMR (400 MHz, DMSO-d₆, δ/ppm): 8.63 (br. s, 1H), 4.67 (s, 2H), 3.97 (t, 2H), 3.60 (t, 2H), 3.50-3.43 (m, 2H), 3.31-3.26 (m, 2H), 3.23 (s, 3H), 2.59 (tt, 1H), 2.40 (s, 3H), 1.04-0.96 (m, 2H), 0.70-0.63 (m, 2H).

LC/MS (Method 4, ESIpos): Rₜ=0.72 min, m/z=405 [M+H]⁺.

### Example 28

### 3-Cyclopropyl-1-(2-methoxyethyl)-5-methyl-6-[(2-oxo-2,3-dihydro-1H-imidazol-1-yl)methyl]thieno[2,3-d]pyrimidine-2,4(1H,3H)-dione

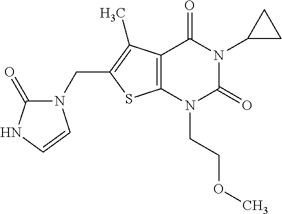 [SMILES: COCCn1c(=O)n(C2CC2)c(=O)c2c(C)c(Cn3cc[nH]c3=O)sc21]

To a solution of 1.06 g (1.59 mmol, 66% purity) of the compound from Ex. 154A in a mixture of 3.2 ml of water and 11.6 ml of methanol were added 3.2 ml (1.58 mmol) of 0.5 M hydrochloric acid. After the reaction mixture had been stirred at RT for 16 h, it was separated into its components directly by means of preparative HPLC (Method 13). Concentration and drying of the product fraction under high vacuum gave 392 mg (63% of theory) of the title compound.

¹H-NMR (400 MHz, DMSO-d₆, δ/ppm): 9.98 (s, 1H), 6.41 (dd, 1H), 6.33 (dd, 1H), 4.77 (s, 2H), 3.96 (dd, 2H), 3.59 (dd, 2H), 3.17 (s, 3H), 2.62-2.56 (m, 1H), 2.43 (s, 3H), 1.02-0.97 (m, 2H), 0.69-0.64 (m, 2H).

LC/MS (Method 1, ESIneg): Rₜ=1.04 min, m/z=375 [M−H]⁻.

### Example 29

### 3-Cyclopropyl-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione

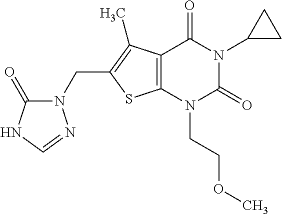 [SMILES: COCCn1c(=O)n(C2CC2)c(=O)c2c(C)c(Cn3nc[nH]c3=O)sc21]

120 mg (0.236 mmol, 92% purity) of the compound from Ex. 250A were dissolved in a mixture of 5.6 ml each of methanol and trimethyl orthoformate, and 590 μl (2.36 mmol) of a 4 M solution of hydrogen chloride in dioxane were added at RT. After 1 h and after 2 h, a further 295 μl (1.18 mmol) each time of the 4 M solution of hydrogen chloride in dioxane were added. After a total of 16 h, the reaction mixture was diluted with water and extracted with ethyl acetate. The organic extract was concentrated and the residue was purified by means of preparative HPLC (Method 11). After the product fractions had been concentrated, the residue was dissolved in ethyl acetate and washed with saturated sodium hydrogencarbonate solution. After concentration and drying under high vacuum again, 42 mg (47% of theory) of the title compound were obtained.

¹H-NMR (400 MHz, DMSO-d₆, δ/ppm): 11.58 (br. s, 1H), 7.83 (s, 1H), 4.91 (s, 2H), 3.96 (t, 2H), 3.60 (t, 2H), 3.22 (s, 3H), 2.63-2.56 (m, 1H), 2.43 (s, 3H), 1.07-0.91 (m, 2H), 0.75-0.60 (m, 2H).

LC/MS (Method 1, ESIpos): Rₜ=1.00 min, m/z=378.12 [M+H]⁺.

### Example 30

### 3-Cyclopropyl-1-(2-methoxyethyl)-5-methyl-6-[(2-oxoimidazolidin-1-yl)dideuteromethyl]thieno[2,3-d]pyrimidine-2,4(1H,3H)-dione

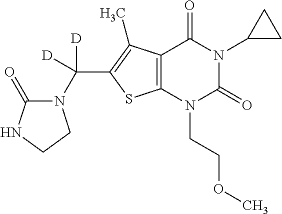 [SMILES: [2H]C([2H])(c1sc2c(c1C)c(=O)n(C1CC1)c(=O)n2CCOC)N1CCNC1=O]

Analogously to the method described in Ex. 16, 570 mg (1.83 mmol) of the compound from Ex. 293A and 628 mg (7.30 mmol) of imidazolidin-2-one were used to prepare 203 mg (28% of theory, 97% purity) of the title compound. The reaction time in this case was about 16 h.

¹H-NMR (400 MHz, DMSO-d₆, δ/ppm): 6.50 (s, 1H), 3.97 (t, 2H), 3.61 (t, 2H), 3.27-3.16 (m, 4H), 3.23 (s, 3H), 2.64-2.55 (m, 1H), 2.37 (s, 3H), 1.06-0.95 (m, 2H), 0.72-0.62 (m, 2H).

LC/MS (Method 1, ESIpos): Rₜ=1.08 min, m/z=381.15 [M+H]⁺.

### Example 31

### 3-Cyclopropyl-1-(2-ethoxyethyl)-5-methyl-6-[(2-oxoimidazolidin-1-yl)methyl]thieno[2,3-d]pyrimidine-2,4(1H,3H)-dione

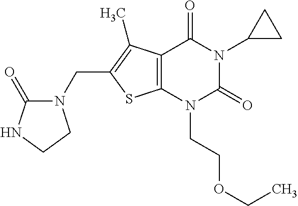 [SMILES: CCOCCn1c(=O)n(C2CC2)c(=O)c2c(C)c(CN3CCNC3=O)sc21]

To a solution of 280 mg (0.611 mmol, 80% purity) of the compound from Ex. 107A and 128 μl (0.917 mmol) of triethylamine in 7 ml of THF were added 119 mg (0.733 mmol) of CDI, and the mixture was stirred at RT for about 16 h. Subsequently, the mixture was concentrated to dryness. The remaining residue was taken up in ethyl acetate and washed successively with saturated sodium hydrogencarbonate solution and saturated sodium chloride solution. After drying over anhydrous magnesium sulfate, the mixture was filtered and concentrated. The solid residue was stirred with a little acetonitrile at RT. The solids were filtered off with suction and dried under high vacuum. The filtrate was purified by means of preparative HPLC (Method 11). The product fraction was concentrated, dried under high vacuum and combined with the solids obtained beforehand. A total of 102 mg (42% of theory) of the title compound were obtained.

¹H-NMR (400 MHz, DMSO-d₆, δ/ppm): 6.51 (s, 1H), 4.33 (s, 2H), 3.97 (t, 2H), 3.63 (t, 2H), 3.43 (q, 2H), 3.27-3.14 (m, 4H), 2.63-2.56 (m, 1H), 2.37 (s, 3H), 1.08-0.94 (m, 2H), 1.04 (t, 3H), 0.72-0.63 (m, 2H).

LC/MS (Method 1, ESIpos): Rₜ=1.24 min, m/z=393.16 [M+H]⁺.

### Example 32

### 3-Cyclopropyl-1-(2-ethoxyethyl)-5-methyl-6-[(5-oxo-4,5-dihydro-1H-1,2,4-triazol-1-yl)methyl]thieno[2,3-d]pyrimidine-2,4(1H,3H)-dione

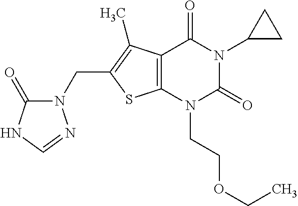 [SMILES: CCOCCn1c(=O)n(C2CC2)c(=O)c2c(C)c(Cn3nc[nH]c3=O)sc21]

Analogously to the method described in Ex. 2, 260 mg (0.513 mmol, 95% purity) of the compound from Ex. 251A were used to prepare 123 mg (61% of theory) of the title compound.

¹H-NMR (400 MHz, DMSO-d₆, δ/ppm): 11.58 (br. s, 1H), 7.82 (s, 1H), 4.91 (s, 2H), 3.95 (br. t, 2H), 3.62 (t, 2H), 3.42 (q, 2H), 2.64-2.56 (m, 1H), 2.43 (s, 3H), 1.08-0.91 (m, 2H), 1.02 (t, 3H), 0.74-0.58 (m, 2H).

LC/MS (Method 1, ESIpos): Rₜ=1.12 min, m/z=392.14 [M+H]⁺.

### Example 33

### 3-Cyclopropyl-1-(2-isopropoxyethyl)-5-methyl-6-[(2-oxoimidazolidin-1-yl)methyl]thieno[2,3-d]pyrimidine-2,4(1H,3H)-dione

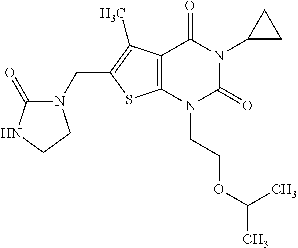 [SMILES: Cc1c(CN2CCNC2=O)sc2c1c(=O)n(C1CC1)c(=O)n2CCOC(C)C]

Analogously to the method described in Ex. 3, 242 mg (0.541 mmol, 85% purity) of the compound from Ex. 108A and 105 mg (0.649 mmol) of CDI were used to prepare 157 mg (71% of theory) of the title compound.

¹H-NMR (400 MHz, DMSO-d₆, δ/ppm): 6.51 (s, 1H), 4.33 (s, 2H), 4.01-3.87 (m, 2H), 3.62 (t, 2H), 3.54 (dt, 1H), 3.26-3.14 (m, 4H), 2.63-2.56 (m, 1H), 2.37 (s, 3H), 1.03-0.98 (m, 2H), 1.01 (d, 6H), 0.74-0.60 (m, 2H).

LC/MS (Method 1, ESIpos): Rₜ=1.34 min, m/z=407.17 [M+H]⁺.

### Example 34

### 3-Cyclopropyl-1-(2-isopropoxyethyl)-5-methyl-6-[(5-oxo-4,5-dihydro-H-1,2,4-triazol-1-yl)methyl]thieno[2,3-d]pyrimidine-2,4(1H,3H)-dione

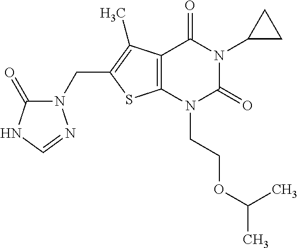 [SMILES: Cc1c(Cn2nc[nH]c2=O)sc2c1c(=O)n(C1CC1)c(=O)n2CCOC(C)C]

Analogously to the method described in Ex. 2, 405 mg (0.817 mmol) of the compound from Ex. 252A were used to prepare 145 mg (43% of theory) of the title compound.

¹H-NMR (400 MHz, DMSO-d₆, δ/ppm): 11.57 (br. s, 1H), 7.82 (s, 1H), 4.91 (s, 2H), 3.92 (br. t, 2H), 3.61 (t, 2H), 3.57-3.47 (m, 1H), 2.59 (dquin, 1H), 2.43 (s, 3H), 1.03-0.96 (m, 2H), 0.99 (d, 6H), 0.72-0.56 (m, 2H).

LC/MS (Method 1, ESIpos): Rₜ=1.23 min, m/z=406.15 [M+H]⁺.

### Example 35

### 3-Cyclopropyl-5-methyl-6-[(2-oxoimidazolidin-1-yl)methyl]-1-[2-(trifluoromethoxy)ethyl]thieno[2,3-d]pyrimidine-2,4(1H,3H)-dione

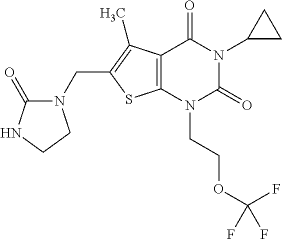 [SMILES: Cc1c(CN2CCNC2=O)sc2c1c(=O)n(C1CC1)c(=O)n2CCOC(F)(F)F]

To a solution of 330 mg (0.650 mmol, 80% purity) of the compound from Ex. 109A and 136 μl (0.974 mmol) of triethylamine in 7 ml of THF were added 126 mg (0.779 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 105 mg (37% of theory) of the title compound were obtained.

¹H-NMR (400 MHz, DMSO-d₆, δ/ppm): 6.52 (s, 1H), 4.37 (t, 2H), 4.34 (s, 2H), 4.15 (t, 2H), 3.27-3.14 (m, 4H), 2.61 (tt, 1H), 2.38 (s, 3H), 1.09-0.94 (m, 2H), 0.76-0.60 (m, 2H).

LC/MS (Method 1, ESIpos): Rₜ=1.43 min, m/z=433.11 [M+H]⁺.

### Example 36

### 3-Cyclopropyl-5-methyl-6-[(5-oxo-4,5-dihydro-1H-1,2,4-triazol-1-yl)methyl]-1-[2-(trifluoromethoxy)ethyl]thieno[2,3-d]pyrimidine-2,4(1H,3H)-dione

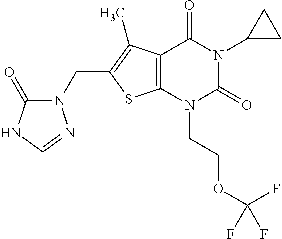 [SMILES: Cc1c(Cn2nc[nH]c2=O)sc2c1c(=O)n(C1CC1)c(=O)n2CCOC(F)(F)F]

360 mg (0.656 mmol, 95% purity) of the compound from Ex. 253A were dissolved in a mixture of 20.5 ml each of methanol and trimethyl orthoformate, and 1.6 ml (6.56 mmol) of a 4 M solution of hydrogen chloride in dioxane were added at RT. After 2 days, the reaction mixture was concentrated and the residue was taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 144 mg (50% of theory) of the title compound were obtained.

¹H-NMR (400 MHz, DMSO-d₆, δ/ppm): 11.59 (br. s, 1H), 7.83 (s, 1H), 4.93 (s, 2H), 4.36 (t, 2H), 4.13 (t, 2H), 2.60 (tt, 1H), 2.44 (s, 3H), 1.09-0.92 (m, 2H), 0.74-0.60 (m, 2H).

LC/MS (Method 1, ESIpos): Rₜ=1.33 min, m/z=432.09 [M+H]⁺.

Crystallization: 20.7 g of the title compound that came from a synthesis on a larger scale were heated to reflux in a mixture of 490 ml of water and 325 ml of ethanol, in the course of which the solid just went completely into solution. Then the heating bath was turned down to 70° C. until the product started to crystallize out. Thereafter, the heating bath was turned back up to 75° C., and the suspension was stirred at this temperature for about 16 h. Subsequently, the temperature of the heating bath was reduced stepwise, and stirring was effected over the following periods of time at the specified temperatures: 5 h 65° C.-16 h 50° C.-2 h 40° C.-2 h 30° C.-90 min RT. Thereafter, the product was filtered off with suction, washed with 200 ml of water/ethanol (3:2), and dried first at 10 mbar and 40° C. for 1 h and then under high vacuum at RT. 17.7 g (85% of theory) of the title compound were obtained in crystalline form.

¹H-NMR (400 MHz, DMSO-d₆, δ/ppm): 11.59 (br. s, 1H), 7.83 (s, 1H), 4.93 (s, 2H), 4.36 (t, 2H), 4.13 (t, 2H), 2.60 (tt, 1H), 2.44 (s, 3H), 1.09-0.93 (m, 2H), 0.74-0.59 (m, 2H).

LC/MS (Method 1, ESIpos): Rₜ=1.33 min, m/z=432.09 [M+H]⁺.

Melting point: 199° C.

### Example 37

### 3-Cyclopropyl-5-methyl-6-[(2-oxoimidazolidin-1-yl)methyl]-1-(tetrahydrofuran-2-ylmethyl)thieno[2,3-d]pyrimidine-2,4(1H,3H)-dione (racemate)

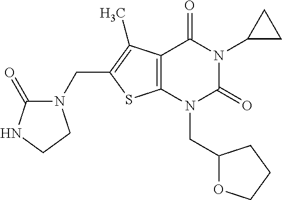 [SMILES: Cc1c(CN2CCNC2=O)sc2c1c(=O)n(C1CC1)c(=O)n2CC1CCCO1]

Analogously to Ex. 5, 300 mg (0.674 mmol) of the compound from Ex. 110A in 30 ml of dioxane were reacted with 169 mg (1.01 mmol) of CDI. 101 mg (36% of theory) of the title compound were obtained.

¹H-NMR (400 MHz, DMSO-d₆, δ/ppm): 6.53 (s, 1H), 4.32 (s, 2H), 4.23-4.15 (m, 1H), 3.99 (dd, 1H), 3.78-3.69 (m, 1H), 3.69-3.56 (m, 2H), 3.27-3.15 (m, 4H), 2.59 (tt, 1H), 2.37 (s, 3H), 2.02-1.91 (m, 1H), 1.91-1.74 (m, 2H), 1.69-1.59 (m, 1H), 1.04-0.96 (m, 2H), 0.70-0.63 (m, 2H).

LC/MS (Method 4, ESIpos): Rₜ=0.85 min, m/z=405 [M+H]⁺.

### Example 38

### 3-Cyclopropyl-5-methyl-6-[(2-oxoimidazolidin-1-yl)methyl]-1-[(2R)-tetrahydrofuran-2-ylmethyl]thieno[2,3-d]pyrimidine-2,4(1H,3H)-dione

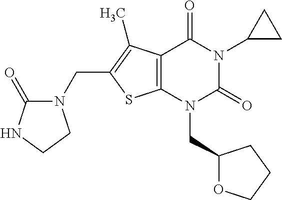 [SMILES: Cc1c(CN2CCNC2=O)sc2c1c(=O)n(C1CC1)c(=O)n2C[C@H]1CCCO1]

Analogously to the method described in Ex. 3, 170 mg (0.404 mmol, 90% purity) of the compound from Ex. 111A and 79 mg (0.485 mmol) of CDI were used to prepare 79 mg (48% of theory) of the title compound.

¹H-NMR (400 MHz, DMSO-d₆, δ/ppm): 6.51 (s, 1H), 4.33 (s, 2H), 4.25-4.15 (m, 1H), 3.99 (dd, 1H), 3.78-3.70 (m, 1H), 3.70-3.56 (m, 2H), 3.27-3.15 (m, 4H), 2.64-2.56 (m, 1H), 2.37 (s, 3H), 2.03-1.73 (m, 3H), 1.71-1.57 (m, 1H), 1.07-0.94 (m, 2H), 0.72-0.61 (m, 2H).

LC/MS (Method 1, ESIpos): Rₜ=1.21 min, m/z=405.16 [M+H]⁺.

Specific optical rotation: [c]_{D}20=−26.8·ml·dm⁻¹·g⁻¹ (DMSO).

### Example 39

### 3-Cyclopropyl-5-methyl-6-[(2-oxoimidazolidin-1-yl)methyl]-1-[(2S)-tetrahydrofuran-2-ylmethyl]thieno[2,3-d]pyrimidine-2,4(1H,3H)-dione

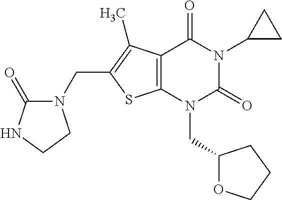 [SMILES: Cc1c(CN2CCNC2=O)sc2c1c(=O)n(C1CC1)c(=O)n2C[C@@H]1CCCO1]

Analogously to the method described in Ex. 3, 150 mg (0.357 mmol, 90% purity) of the compound from Ex. 112A and 69 mg (0.428 mmol) of CDI were used to prepare 58 mg (40% of theory) of the title compound.

¹H-NMR (400 MHz, DMSO-d₆, δ/ppm): 6.51 (s, 1H), 4.33 (s, 2H), 4.25-4.14 (m, 1H), 3.99 (dd, 1H), 3.79-3.70 (m, 1H), 3.70-3.55 (m, 2H), 3.27-3.16 (m, 4H), 2.64-2.56 (m, 1H), 2.37 (s, 3H), 2.03-1.74 (m, 3H), 1.70-1.58 (m, 1H), 1.07-0.92 (m, 2H), 0.72-0.60 (m, 2H).

LC/MS (Method 1, ESIpos): Rₜ=1.21 min, m/z=405.16 [M+H]⁺.

Specific optical rotation: [c]D₂₀=+25.5°·ml·dm⁻¹·g⁻¹ (DMSO).

### Example 40

### 3-Cyclopropyl-5-methyl-1-(tetrahydrofuran-2-ylmethyl)-6-[(2-thioxoimidazolidin-1-yl)methyl]thieno[2,3-d]pyrimidine-2,4(1H,3H)-dione (racemate)

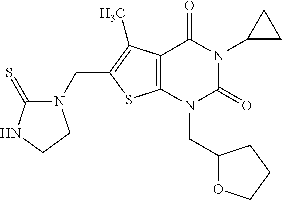 [SMILES: Cc1c(CN2CCNC2=S)sc2c1c(=O)n(C1CC1)c(=O)n2CC1CCCO1]

Analogously to Ex. 6, 100 mg (0,225 mmol) of the compound from Ex. 110A in 15 ml of dioxane were reacted with 63 mg (0.337 mmol) of 1,1′-thiocarbonyldiimidazole. 30 mg (31% of theory) of the title compound were obtained.

¹H-NMR (400 MHz, DMSO-d₆, δ/ppm): 8.34 (s, 1H), 4.80 (s, 2H), 4.23-4.15 (m, 1H), 3.99 (dd, 1H), 3.78-3.70 (m, 1H), 3.69-3.56 (m, 2H), 3.55-3.47 (m, 2H), 3.43-3.36 (m, 2H), 2.63-2.56 (m, 1H), 2.41 (s, 3H), 2.02-1.91 (m, 1H), 1.91-1.74 (m, 2H), 1.69-1.59 (m, 1H), 1.04-0.96 (m, 2H), 0.69-0.63 (m, 2H).

LC/MS (Method 4, ESIpos): Rₜ=0.95 min, m/z=421 [M+H]⁺.

### Example 41

### [1-{[3-Cyclopropyl-5-methyl-2,4-dioxo-1-(tetrahydrofuran-2-ylmethyl)-1,2,3,4-tetrahydrothieno[2,3-d]pyrimidin-6-yl]methyl}imidazolidin-2-ylidene]cyanamide (racemate)

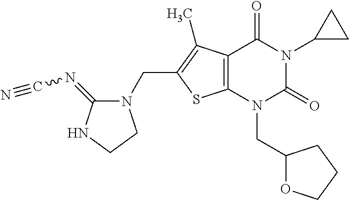 [SMILES: Cc1c(CN2CCNC2=NC#N)sc2c1c(=O)n(C1CC1)c(=O)n2CC1CCCO1]

570 mg (1.21 mmol, 80% purity) of the compound from Ex. 110A were dissolved in 16 ml of DMF, and 264 mg (1.81 mmol) of dimethyl N-cyanodithioiminocarbonate and 333 mg (2.41 mmol) of potassium carbonate were added. The mixture was stirred at 80° C. in a microwave oven (Biotage Initiator with dynamic control of irradiation power) for 4 h. Thereafter, the reaction mixture was diluted with ethyl acetate and washed successively with saturated sodium carbonate solution and saturated sodium chloride solution. After drying over anhydrous magnesium sulfate, the mixture was filtered and concentrated. The crude product was purified by preparative HPLC (Method 11). The product fractions were combined, concentrated by evaporation and dried under high vacuum. 215 mg (40% of theory, 97% purity) of the title compound were obtained.

¹H-NMR (500 MHz, DMSO-d₆, δ/ppm): 8.06 (s, 1H), 4.46 (s, 2H), 4.23-4.17 (m, 1H), 4.01 (br. dd, 1H), 3.75 (q, 1H), 3.70-3.57 (m, 2H), 3.48-3.36 (m, 4H), 2.65-2.56 (m, 1H), 2.38 (s, 3H), 2.03-1.75 (m, 3H), 1.72-1.59 (m, 1H), 1.03-0.97 (m, 2H), 0.69-0.64 (m, 2H).

LC/MS (Method 6, ESIpos): Rₜ=0.99 min, m/z=429 [M+H]⁺.

### Example 42

### Methyl [1-{[3-cyclopropyl-5-methyl-2,4-dioxo-1-(tetrahydrofuran-2-ylmethyl)-1,2,3,4-tetrahydrothieno[2,3-d]pyrimidin-6-yl]methyl}imidazolidin-2-ylidene]carbamate (racemate)

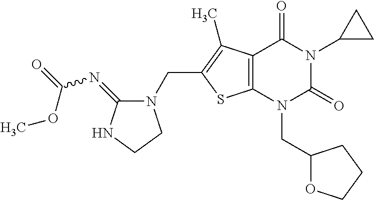 [SMILES: COC(=O)N=C1NCCN1Cc1sc2c(c1C)c(=O)n(C1CC1)c(=O)n2CC1CCCO1]

510 mg (1.08 mmol, 80% purity) of the compound from Ex. 110A and 300 μl (2.16 mmol) of triethylamine were dissolved in 20 ml of dichloromethane, and a solution of 336 mg (2.16 mmol) of methyl (dichloromethylene)carbamate in 5 ml of dichloromethane was added dropwise. After the reaction mixture had been stirred at RT for about 16 h, it was concentrated to dryness. The residue was prepurified by means of preparative HPLC (Method 11). Since the product fraction was still contaminated, there followed a second chromatography operation by means of MPLC (Biotage Isolera One, SNAP KP-Sil cartridge, 50 g of silica gel, eluent: ethyl acetate). Concentration of the product fraction and drying of the residue under high vacuum gave 166 mg (31% of theory, 95% purity) of the title compound.

¹H-NMR (400 MHz, DMSO-d₆, δ/ppm): 8.01 (s, 1H), 4.53 (s, 2H), 4.25-4.15 (m, 1H), 4.00 (dd, 1H), 3.77-3.68 (m, 1H), 3.66-3.55 (m, 2H), 3.53 (s, 3H), 3.49-3.41 (m, 2H), 3.35-3.28 (m, 2H, substantially concealed by water signal), 2.64-2.56 (m, 1H), 2.40 (s, 3H), 2.02-1.74 (m, 3H), 1.70-1.59 (m, 1H), 1.04-0.95 (m, 2H), 0.71-0.62 (m, 2H).

LC/MS (Method 1, ESIpos): Rₜ=1.16 min, m/z=462.18 [M+H]⁺.

### Example 43

### Methyl [1-{[3-cyclopropyl-5-methyl-2,4-dioxo-1-(tetrahydrofuran-2-ylmethyl)-1,2,3,4-tetrahydrothieno[2,3-d]pyrimidin-6-yl]methyl}imidazolidin-2-ylidene]carbamate (enantiomer 1)

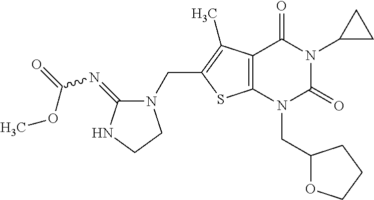 [SMILES: COC(=O)N=C1NCCN1Cc1sc2c(c1C)c(=O)n(C1CC1)c(=O)n2CC1CCCO1]

161 mg of the racemic compound from Ex. 42 were dissolved in a mixture of 4 ml of ethanol and 2 ml of acetonitrile and, in 20 portions, separated into the enantiomers via preparative HPLC on a chiral phase [column: YMC Chiralart SC, 5 μm, 250 mm×20 mm; eluent: ethanol; flow rate: 15 ml/min; temperature: 55° C.; detection: 220 nm]. After concentration of the product fractions and drying of the solids under high vacuum, 66 mg (81% of theory) of enantiomer 1 were obtained (>99.0% ee, chiral analytical HPLC).

¹H-NMR (400 MHz, DMSO-d₆, δ/ppm): 8.01 (s, 1H), 4.53 (s, 2H), 4.25-4.15 (m, 1H), 4.00 (dd, 1H), 3.77-3.68 (m, 1H), 3.66-3.56 (m, 2H), 3.53 (s, 3H), 3.48-3.41 (m, 2H), 3.35-3.28 (m, 2H, substantially concealed by water signal), 2.60 (tt, 1H), 2.40 (s, 3H), 2.02-1.74 (m, 3H), 1.71-1.59 (m, 1H), 1.05-0.96 (m, 2H), 0.71-0.61 (m, 2H).

Chiral analytical HPLC [column: Daicel Chiralpak IC, 5 μm, 250 mm×4.6 mm; Laufmittel: ethanol; flow rate: 1 ml/min; temperature: 55° C.; detection: 220 nm]: Rₜ=13.93 min.

### Example 44

### Methyl [1-{[3-cyclopropyl-5-methyl-2,4-dioxo-1-(tetrahydrofuran-2-ylmethyl)-1,2,3,4-tetrahydrothieno[2,3-d]pyrimidin-6-yl]methyl}imidazolidin-2-ylidene]carbamate (enantiomer 2)

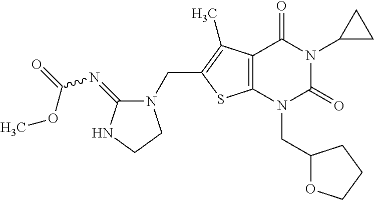 [SMILES: COC(=O)N=C1NCCN1Cc1sc2c(c1C)c(=O)n(C1CC1)c(=O)n2CC1CCCO1]

161 mg of the racemic compound from Ex. 42 were dissolved in a mixture of 4 ml of ethanol and 2 ml of acetonitrile and, in 20 portions, separated into the enantiomers via preparative HPLC on a chiral phase [column: YMC Chiralart SC, 5 μm, 250 mm×20 mm; eluent: ethanol; flow rate: 15 ml/min; temperature: 55° C.; detection: 220 nm]. After concentration of the product fractions and drying of the solids under high vacuum, 59 mg (73% of theory) of enantiomer 2 were obtained (>99.0% ee, chiral analytical HPLC).

¹H-NMR (400 MHz, DMSO-d₆, δ/ppm): 8.01 (s, 1H), 4.53 (s, 2H), 4.25-4.14 (m, 1H), 4.00 (dd, 1H), 3.77-3.68 (m, 1H), 3.66-3.56 (m, 2H), 3.53 (s, 3H), 3.49-3.41 (m, 2H), 3.35-3.28 (m, 2H), 2.63-2.56 (m, 1H), 2.40 (s, 3H), 2.02-1.74 (m, 3H), 1.70-1.59 (m, 1H), 1.07-0.92 (m, 2H), 0.72-0.60 (m, 2H).

Chiral analytical HPLC [column: Daicel Chiralpak IC, 5 μm, 250 mm×4.6 mm; eluent: ethanol; flow rate: 1 ml/min; temperature: 55° C.; detection: Rₜ=15.76 min.

### Example 45

### 3-Cyclopropyl-6-[(2,3-dioxopiperazin-1-yl)methyl]-5-methyl-1-(tetrahydrofuran-2-ylmethyl)thieno[2,3-d]pyrimidine-2,4(1H,3H)-dione (racemate)

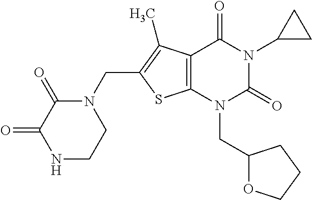 [SMILES: Cc1c(CN2CCNC(=O)C2=O)sc2c1c(=O)n(C1CC1)c(=O)n2CC1CCCO1]

Analogously to Ex. 8, 100 mg (0.225 mmol) of the compound from Ex. 110A in 5 ml of ethanol were reacted with 331 mg (2.246 mmol) of diethyl oxalate. 27 mg (27% of theory) of the title compound were obtained.

¹H-NMR (400 MHz, DMSO-d₆, δ/ppm): 8.63 (br. s, 1H), 4.67 (s, 2H), 4.24-4.15 (m, 1H), 4.00 (dd, 1H), 3.77-3.70 (m, 1H), 3.69-3.56 (m, 2H), 3.50-3.43 (m, 2H), 3.31 (br. d, 2H), 2.60 (tt, 1H), 2.40 (s, 3H), 2.02-1.91 (m, 1H), 1.91-1.75 (m, 2H), 1.69-1.59 (m, 1H), 1.04-0.97 (m, 2H), 0.69-0.63 (m, 2H).

LC/MS (Method 4, ESIneg): Rₜ=0.77 min, m/z=477 [M−H+HCO₂H]⁻.

### Example 46

### 3-Cyclopropyl-5-methyl-6-[(2-oxo-2,3-dihydro-H-imidazol-1-yl)methyl]-1-(tetrahydrofuran-2-ylmethyl)thieno[2,3-d]pyrimidine-2,4(1H,3H)-dione (racemate)

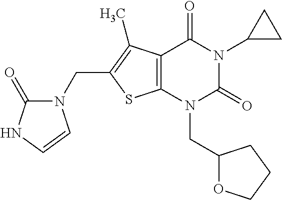 [SMILES: Cc1c(Cn2cc[nH]c2=O)sc2c1c(=O)n(C1CC1)c(=O)n2CC1CCCO1]

To a solution of 1.1 g (1.38 mmol, 60% purity) of the compound from Ex. 155A in a mixture of 2.8 ml of water and 10 ml of methanol were added 2.7 ml (1.36 mmol) of 0.5 M hydrochloric acid. After the reaction mixture had been stirred at RT for 40 h, it was separated into its components directly by means of preparative HPLC (Method 13). Concentration and drying of the product fraction under high vacuum gave 151 mg (26% of theory) of the title compound.

¹H-NMR (400 MHz, DMSO-d₆, δ/ppm): 9.98 (s, 1H), 6.41 (dd, 1H), 6.33 (dd, 1H), 4.77 (s, 2H), 4.21-4.15 (m, 1H), 3.99 (dd, 1H), 3.75-3.70 (m, 1H), 3.66-3.57 (m, 2H), 2.62-2.58 (m, 1H), 2.44 (s, 3H), 1.98-1.75 (m, 3H), 1.68-1.59 (m, 1H), 1.03-0.97 (m, 2H), 0.68-0.64 (m, 2H).

LC/MS (Method 1, ESIneg): Rₜ=1.11 min, m/z=401 [M−H]⁻.

### Example 47

### 3-Cyclopropyl-5-methyl-6-[(2-oxo-2,3-dihydro-H-imidazol-1-yl)methyl]-1-(tetrahydrofuran-2-ylmethyl)thieno[2,3-d]pyrimidine-2,4(1H,3H)-dione (enantiomer 1)

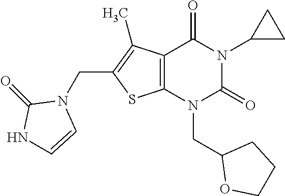 [SMILES: Cc1c(Cn2cc[nH]c2=O)sc2c1c(=O)n(C1CC1)c(=O)n2CC1CCCO1]

151 mg of the racemic compound from Ex. 46 were dissolved in 3 ml of a methanol/TBME/dichloromethane mixture (3:2:1) and separated into the enantiomers by means of preparative HPLC on a chiral phase [column: Daicel Chiralpak ID 5 μm 250 mm×20 mm; eluent: TBME/Methanol 1:1; flow rate: 15 ml/min; temperature: 30° C.; detection: 220 nm]. The product fractions were concentrated on a rotary evaporator, admixed with tert-butanol and freeze-dried. 50 mg (66% of theory) of the title compound (enantiomer 1) and 53 mg (70% of theory) of enantiomer 2 (see Ex. 48) were obtained.

¹H-NMR (400 MHz, DMSO-d₆, δ/ppm): 9.98 (s, 1H), 6.41 (dd, 1H), 6.33 (dd, 1H), 4.77 (s, 2H), 4.21-4.15 (m, 1H), 3.99 (dd, 1H), 3.75-3.70 (m, 1H), 3.66-3.57 (m, 2H), 2.62-2.58 (m, 1H), 2.44 (s, 3H), 1.98-1.75 (m, 3H), 1.68-1.59 (m, 1H), 1.03-0.97 (m, 2H), 0.68-0.64 (m, 2H).

LC/MS (Method 1, ESIneg): Rₜ=1.13 min, m/z=401 [M−H]⁻.

Chiral analytical HPLC [column: Daicel Chiralpak ID 5 μm 100 mm×4.6 mm; eluent: methanol+0.2% acetic acid/TBME 15:85; flow rate: 1.0 ml/min; temperature: 30° C.; injection: 235 nm]: Rₜ=5.90 min.

### Example 48

### 3-Cyclopropyl-5-methyl-6-[(2-oxo-2,3-dihydro-H-imidazol-1-yl)methyl]-1-(tetrahydrofuran-2-ylmethyl)thieno[2,3-d]pyrimidine-2,4(1H,3H)-dione (enantiomer 2)

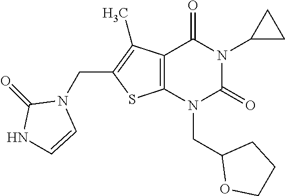 [SMILES: Cc1c(Cn2cc[nH]c2=O)sc2c1c(=O)n(C1CC1)c(=O)n2CC1CCCO1]

The title compound (53 mg) was obtained as the second enantiomer in the preparative HPLC separation of the racemate from Ex. 46 described in Ex. 47.

¹H-NMR (400 MHz, DMSO-d₆, δ/ppm): 9.98 (s, 1H), 6.41 (dd, 1H), 6.33 (dd, 1H), 4.77 (s, 2H), 4.21-4.15 (m, 1H), 3.99 (dd, 1H), 3.75-3.70 (m, 1H), 3.66-3.57 (m, 2H), 2.62-2.58 (m, 1H), 2.44 (s, 3H), 1.98-1.75 (m, 3H), 1.68-1.59 (m, 1H), 1.03-0.97 (m, 2H), 0.68-0.64 (m, 2H).

LC/MS (Method 1, ESIneg): Rₜ=1.11 min, m/z=401 [M−H]⁻.

Chiral analytical HPLC [column: Daicel Chiralpak ID 5 μm 100 mm×4.6 mm; eluent: methanol+0.2% acetic acid/TBME 15:85; flow rate: 1.0 ml/min; temperature: 30° C.; injection: 5 μl; DAD: 235 nm]: Rₜ=6.69 min.

### Example 49

### 3-Cyclopropyl-5-methyl-6-[(5-oxo-4,5-dihydro-1H-1,2,4-triazol-1-yl)methyl]-1-(tetrahydrofuran-2-ylmethyl)thieno[2,3-d]pyrimidine-2,4(1H,3H)-dione (racemate)

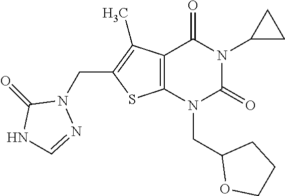 [SMILES: Cc1c(Cn2nc[nH]c2=O)sc2c1c(=O)n(C1CC1)c(=O)n2CC1CCCO1]

358 mg (0.689 mmol, 95% purity) of the compound from Ex. 254A were dissolved in a mixture of 15 ml each of methanol and trimethyl orthoformate, and 1.7 ml (6.89 mmol) of a 4 M solution of hydrogen chloride in dioxane were added at RT. After 1 h and after 2 h, a further 0.85 ml (3.45 mmol) each time of the 4 M solution of hydrogen chloride in dioxane were added. After a total of 16 h, the reaction mixture was diluted with water and extracted with ethyl acetate. The organic extract was concentrated and the residue was then stirred with a little acetonitrile at RT. The solids were removed and dried under high vacuum. The filtrate was concentrated, and the residue was purified by means of preparative HPLC (Method 11). After concentration of the product fraction and drying under high vacuum, the residue was combined with the solids obtained beforehand. A total of 195 mg (70% of theory) of the title compound were obtained.

¹H-NMR (400 MHz, DMSO-d₆, δ/ppm): 11.59 (broad, 1H), 7.82 (s, 1H), 4.91 (s, 2H), 4.24-4.13 (m, 1H), 3.99 (dd, 1H), 3.77-3.69 (m, 1H), 3.68-3.55 (m, 2H), 2.60 (tt, 1H), 2.43 (s, 3H), 2.03-1.74 (m, 3H), 1.71-1.58 (m, 1H), 1.06-0.93 (m, 2H), 0.74-0.60 (m, 2H).

LC/MS (Method 1, ESIpos): Rₜ=1.06 min, m/z=404.14 [M+H]⁺.

### Example 50

### 3-Cyclopropyl-5-methyl-6-[(5-oxo-4,5-dihydro-1H-1,2,4-triazol-1-yl)methyl]-1-[(2R)-tetrahydrofuran-2-ylmethyl]thieno[2,3-d]pyrimidine-2,4(1H,3H)-dione

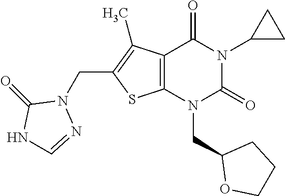 [SMILES: Cc1c(Cn2nc[nH]c2=O)sc2c1c(=O)n(C1CC1)c(=O)n2C[C@H]1CCCO1]

255 mg (0.465 mmol, 90% purity) of the compound from Ex. 255A were dissolved in a mixture of 10 ml each of methanol and trimethyl orthoformate, and 1.2 ml (4.65 mmol) of a 4 M solution of hydrogen chloride in dioxane were added at RT. After about 16 h, the reaction mixture was concentrated and the residue was taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 121 mg (64% of theory) of the title compound were obtained.

¹H-NMR (400 MHz, DMSO-d₆, δ/ppm): 11.58 (br. s, 1H), 7.83 (s, 1H), 4.91 (s, 2H), 4.22-4.14 (m, 1H), 3.99 (br. dd, 1H), 3.73 (q, 1H), 3.68-3.55 (m, 2H), 2.63-2.56 (m, 1H), 2.43 (s, 3H), 2.03-1.74 (m, 3H), 1.71-1.57 (m, 1H), 1.03-0.97 (m, 2H), 0.69-0.63 (m, 2H).

LC/MS (Method 1, ESIpos): Rₜ=1.10 min, m/z=404.14 [M+H]⁺.

Specific optical rotation: [α]_{D}20=−23.4·ml·dm⁻¹·g⁻¹ (DMSO).

### Example 51

### 3-Cyclopropyl-5-methyl-6-[(5-oxo-4,5-dihydro-1H-1,2,4-triazol-1-yl)methyl]-1-[(2S)-tetrahydrofuran-2-ylmethyl]thieno[2,3-d]pyrimidine-2,4(1H, 3H)-dione

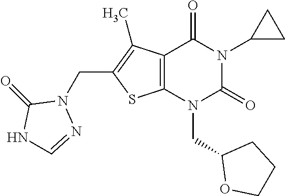 [SMILES: Cc1c(Cn2nc[nH]c2=O)sc2c1c(=O)n(C1CC1)c(=O)n2C[C@@H]1CCCO1]

Analogously to the method described in Ex. 50, 210 mg (0.383 mmol, 90% purity) of the compound from Ex. 256A were used to prepare 107 mg (69% of theory) of the title compound.

¹H-NMR (400 MHz, DMSO-d₆, δ/ppm): 11.58 (br. s, 1H), 7.82 (s, 1H), 4.91 (s, 2H), 4.22-4.14 (m, 1H), 3.99 (dd, 1H), 3.73 (q, 1H), 3.68-3.55 (m, 2H), 2.63-2.56 (m, 1H), 2.43 (s, 3H), 2.04-1.75 (m, 3H), 1.70-1.57 (m, 1H), 1.06-0.93 (m, 2H), 0.68-0.63 (m, 2H).

LC/MS (Method 2, ESIpos): Rₜ=0.63 min, m/z=404 [M+H]⁺.

Specific optical rotation: [α]_{D}²⁰=+30.0°·ml·dm⁻¹·g⁻¹ (DMSO).

### Example 52

### 1,5-Dimethyl-3-(1-methylcyclopropyl)-6-[(2-oxoimidazolidin-1-yl)methyl]thieno[2,3-d]pyrimidine-2,4(1H,3H)-dione

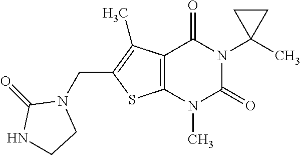 [SMILES: Cc1c(CN2CCNC2=O)sc2c1c(=O)n(C1(C)CC1)c(=O)n2C]

Analogously to the method described in Ex. 3, 247 mg (0.689 mmol, 90% purity) of the compound from Ex. 113A and 134 mg (0.827 mmol) of CDI were used to prepare 164 mg (68% of theory) of the title compound.

¹H-NMR (400 MHz, DMSO-d₆, δ/ppm): 6.51 (s, 1H), 4.34 (s, 2H), 3.31 (s, 3H), 3.27-3.14 (m, 4H), 2.39 (s, 3H), 1.33 (s, 3H), 0.95-0.77 (m, 4H).

LC/MS (Method 2, ESIpos): Rₜ=0.66 min, m/z=349 [M+H]⁺.

### Example 53

### 1,5-Dimethyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione

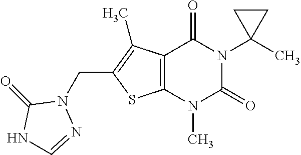 [SMILES: Cc1c(Cn2nc[nH]c2=O)sc2c1c(=O)n(C1(C)CC1)c(=O)n2C]

Analogously to the method described in Ex. 50, 275 mg (0.597 mmol, 95% purity) of the compound from Ex. 257A were used to prepare 153 mg (73% of theory) of the title compound.

¹H-NMR (400 MHz, DMSO-d₆, δ/ppm): 11.58 (br. s, 1H), 7.83 (s, 1H), 4.93 (s, 2H), 3.36 (s, 3H), 2.45 (s, 3H), 1.32 (s, 3H), 0.98-0.73 (m, 4H).

LC/MS (Method 1, ESIpos): Rₜ=1.08 min, m/z=348.11 [M+H]⁺.

### Example 54

### 1-Butyl-5-methyl-3-(1-methylcyclopropyl)-6-[(2-oxoimidazolidin-1-yl)methyl]thieno[2,3-d]pyrimidine-2,4(1H,3H)-dione

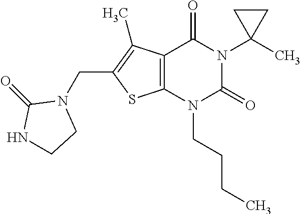 [SMILES: CCCCn1c(=O)n(C2(C)CC2)c(=O)c2c(C)c(CN3CCNC3=O)sc21]

To a solution of 295 mg (0.728 mmol, 90% purity) of the compound from Ex. 114A and 152 μl (1.09 mmol) of triethylamine in 8 ml of THF were added 142 mg (0.874 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 179 mg (63% of theory) of the title compound were obtained.

¹H-NMR (400 MHz, DMSO-d₆, δ/ppm): 6.51 (s, 1H), 4.34 (s, 2H), 3.99-3.65 (m, 2H), 3.28-3.15 (m, 4H), 2.38 (s, 3H), 1.63 (quin, 2H), 1.42-1.26 (m, 2H), 1.33 (s, 3H), 0.91 (t, 3H), 0.89-0.78 (m, 4H).

LC/MS (Method 2, ESIpos): Rₜ=0.86 min, m/z=391 [M+H]⁺.

### Example 55

### 1-Butyl-5-methyl-3-(1-methylcyclopropyl)-6-[(5-oxo-4,5-dihydro-1H-1,2,4-triaiazol-1-yl)methyl]thieno[2,3-d]pyrimidine-2,4(1H,3H)-dione

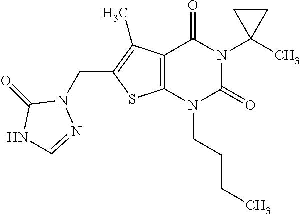 [SMILES: CCCCn1c(=O)n(C2(C)CC2)c(=O)c2c(C)c(Cn3nc[nH]c3=O)sc21]

Analogously to the method described in Ex. 2, 320 mg (0.594 mmol, 89% purity) of the compound from Ex. 258A were used to prepare 174 mg (75% of theory) of the title compound.

¹H-NMR (400 MHz, DMSO-d₆, δ/ppm): 11.59 (br. s, 1H), 7.83 (s, 1H), 4.92 (s, 2H), 3.98-3.63 (m, 2H), 2.44 (s, 3H), 1.62 (quin, 2H), 1.41-1.25 (m, 2H), 1.32 (s, 3H), 0.97-0.70 (m, 4H), 0.90 (t, 3H).

LC/MS (Method 1, ESIpos): Rₜ=1.51 min, m/z=390.16 [M+H]⁺.

### Example 56

### 5-Methyl-3-(1-methylcyclopropyl)-6-[(2-oxoimidazolidin-1-yl)methyl]-1-(3,3,3-trifluoropropyl)thieno[2,3-d]pyrimidine-2,4(1H,3H)-dione

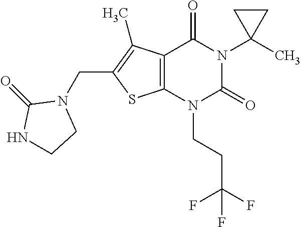 [SMILES: Cc1c(CN2CCNC2=O)sc2c1c(=O)n(C1(C)CC1)c(=O)n2CCC(F)(F)F]

320 mg (0.68 mmol, 86% purity) of the compound from Ex. 115A were dissolved in 5.6 ml of THF, and 132 mg (0.81 mmol) of CDI and 0.14 ml (1.02 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated aqueous sodium hydrogencarbonate solution and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 195 mg (67% of theory) of the title compound.

¹H-NMR (400 MHz, DMSO-d₆, δ/ppm): 6.52 (br. s, 1H), 4.35 (s, 2H), 4.14-3.97 (m, 2H), 3.30-3.18 (m, 4H), 2.80-2.66 (m, 2H), 2.39 (s, 3H), 1.35 (s, 3H), 1.00-0.50 (m, 4H).

LC/MS (Method 2, ESIpos): Rₜ=0.83 min, m/z=431 [M+H]⁺.

### Example 57

### [1-{[5-Methyl-3-(1-methylcyclopropyl)-2,4-dioxo-1-(3,3,3-trifluoropropyl)-1,2,3,4-tetrahydrothieno[2,3-d]pyrimidin-6-yl]methyl}imidazolidin-2-ylidene]cyanamide

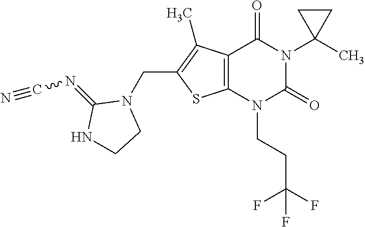 [SMILES: Cc1c(CN2CCNC2=NC#N)sc2c1c(=O)n(C1(C)CC1)c(=O)n2CCC(F)(F)F]

451 mg (0.96 mmol, 86% purity) of the compound from Ex. 115A were dissolved in 38 ml of DMF, and 264 mg (1.91 mmol) of potassium carbonate were added. The mixture was stirred at RT for 15 min, then 210 mg (1.43 mmol) of dimethyl N-cyanodithioiminocarbonate were added, and the reaction mixture was stirred in a microwave oven (Biotage Initiator with dynamic control of irradiation power) at 80° C. for 3 h. Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 129 mg (30% of theory) of the title compound.

¹H-NMR (400 MHz, CD₃OD, δ/ppm): 4.55 (s, 2H), 4.22-4.13 (m, 2H), 3.53 (s, 4H), 2.75-2.65 (m, 2H), 2.47 (s, 3H), 1.41 (s, 3H), 1.02-0.96 (m, 1H), 0.95-0.87 (m, 3H).

LC/MS (Method 2, ESIpos): Rₜ=0.88 min, m/z=455 [M+H]⁺.

### Example 58

### Methyl [1-{[5-methyl-3-(1-methylcyclopropyl)-2,4-dioxo-1-(3,3,3-trifluoropropyl)-1,2,3,4-tetrahydrothieno[2,3-d]pyrimidin-6-yl]methyl}imidazolidin-2-ylidene]carbamate

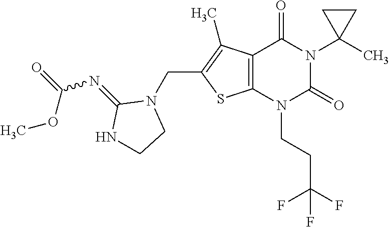

250 mg (0.53 mmol, 86% purity) of the compound from Ex. 115A and 147 μl (1.06 mmol) of triethylamine were dissolved in 24 ml of dichloromethane, and 82 mg (0.53 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 210 mg (82% of theory) of the title compound.

¹H-NMR (400 MHz, CD₃OD, δ/ppm): 4.83 (s, 2H), 4.26-4.16 (m, 2H), 3.95 (s, 3H), 3.83 (t, 2H), 3.68 (t, 2H), 2.78-2.70 (m, 2H), 2.52 (s, 3H), 1.44 (s, 3H), 1.05-1.01 (m, 1H), 0.98-0.92 (m, 3H).

LC/MS (Method 2, ESIpos): Rₜ=0.79 min, m/z=488 [M+H]⁺.

### Example 59

### 6-[(2,3-Dioxopiperazin-1-yl)methyl]-5-methyl-3-(1-methylcyclopropyl)-1-(3,3,3-trifluoropropyl)thieno[2,3-d]pyrimidine-2,4(1H,3H)-dione

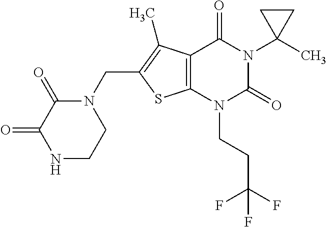 [SMILES: Cc1c(CN2CCNC(=O)C2=O)sc2c1c(=O)n(C1(C)CC1)c(=O)n2CCC(F)(F)F]

230 mg (0.49 mmol, 86% purity) of the compound from Ex. 115A were dissolved in 19 ml of ethanol, and 133 mg (0.90 mmol) of diethyl oxalate were added. The mixture was stirred at 80° C. for 3 days. Thereafter, the reaction solution was concentrated on a rotary evaporator. The residue obtained was purified by preparative HPLC (Method 13). Concentration of the product fractions and drying under high vacuum gave 89 mg (40% of theory) of the title compound.

¹H-NMR (400 MHz, CD₃OD, δ/ppm): 4.80 (s, 2H), 4.22-4.12 (m, 2H), 3.60 (m, 2H), 3.46 (m, 2H), 2.74-2.66 (m, 2H), 2.51 (s, 3H), 1.42 (s, 3H), 1.03-0.97 (m, 1H), 0.95-0.88 (m, 3H).

LC/MS (Method 2, ESIpos): Rₜ=0.75 min, m/z=459 [M+H]⁺.

### Example 60

### 5-Methyl-3-(1-methylcyclopropyl)-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

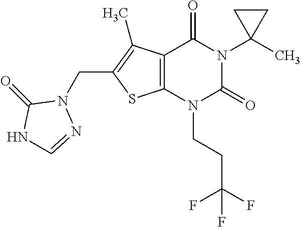 [SMILES: Cc1c(Cn2nc[nH]c2=O)sc2c1c(=O)n(C1(C)CC1)c(=O)n2CCC(F)(F)F]

500 mg (0.71 mmol, 74% purity) of the compound from Ex. 259A were initially charged in 17.5 ml of trimethyl orthoformate and 17.5 ml of methanol, and 1.8 ml (7.14 mmol) of a 4 M solution of hydrogen chloride in dioxane were added. Since the conversion was still incomplete after stirring at RT for 18 h, a further 0.9 ml of 4 M hydrogen chloride in dioxane was added and the mixture was stirred for a further 2.5 h. Thereafter, another 0.9 ml of 4 M hydrogen chloride in dioxane was added, and the mixture was stirred for a further 2.5 h. Then water was added to the reaction mixture, which was extracted with ethyl acetate. The organic phase was concentrated and the residue was purified by means of preparative HPLC (Method 13). The product fractions were combined and concentrated, and the residue was dried under high vacuum. 185 mg (61% of theory) of the title compound were obtained.

¹H-NMR (400 MHz, CD₃OD, δ/ppm): 7.72 (s, 1H), 5.07 (s, 2H), 4.20-4.15 (m, 2H), 3.33-3.32 (m, 4H), 2.75-2.70 (m, 2H), 2.56 (s, 3H), 1.43 (s, 3H), 1.04-0.98 (m, 1H), 0.96-0.90 (m, 3H).

LC/MS (Method 2, ESIpos): Rₜ=0.78 min, m/z=430 [M+H]⁺.

### Example 61

### 1-(Cyclobutylmethyl)-5-methyl-3-(1-methylcyclopropyl)-6-[(2-oxoimidazolidin-1-yl)methyl]thieno[2,3-d]pyrimidine-2,4(1H,3H)-dione

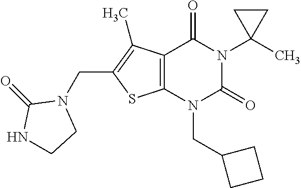 [SMILES: Cc1c(CN2CCNC2=O)sc2c1c(=O)n(C1(C)CC1)c(=O)n2CC1CCC1]

To a solution of 240 mg (0.542 mmol, 85% purity) of the compound from Ex. 116A and 113 μl (0.813 mmol) of triethylamine in 6 ml of THF were added 105 mg (0.650 mmol) of CDI, and the mixture was stirred at RT for 5 days. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 30 mg (13% of theory) of the title compound were obtained.

¹H-NMR (400 MHz, DMSO-d₆, δ/ppm): 6.51 (s, 1H), 4.33 (s, 2H), 4.07-3.72 (m, 2H), 3.21 (br. s, 3H), 2.80-2.64 (m, 1H), 2.37 (s, 3H), 2.00-1.93 (m, 2H), 1.87-1.73 (m, 4H), 1.32 (s, 3H), 0.98-0.67 (m, 4H).

LC/MS (Method 1, ESIpos): Rₜ=1.67 min, m/z=403.18 [M+H]⁺.

### Example 62

### 1-(Cyclobutylmethyl)-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione

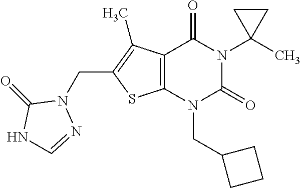 [SMILES: Cc1c(Cn2nc[nH]c2=O)sc2c1c(=O)n(C1(C)CC1)c(=O)n2CC1CCC1]

Analogously to the method described in Ex. 2, 250 mg (0.458 mmol, 90% purity) of the compound from Ex. 260A were used to prepare 65 mg (35% of theory) of the title compound. The reaction time in this case was 6 days.

¹H-NMR (400 MHz, DMSO-d₆, δ/ppm): 11.58 (br. s, 1H), 7.83 (s, 1H), 4.91 (s, 2H), 4.06-3.69 (m, 2H), 2.80-2.64 (m, 1H), 2.43 (s, 3H), 2.00-1.90 (m, 2H), 1.87-1.71 (m, 4H), 1.32 (s, 3H), 0.97-0.67 (m, 4H).

LC/MS (Method 1, ESIpos): Rₜ=1.55 min, m/z=402.16 [M+H]⁺.

### Example 63

### 1-[(2,2-Difluorocyclopropyl)methyl]-5-methyl-3-(1-methylcyclopropyl)-6-[(2-oxoimidazolidin-1-yl)methyl]thieno[2,3-d]pyrimidine-2,4(1H,3H)-dione (racemate)

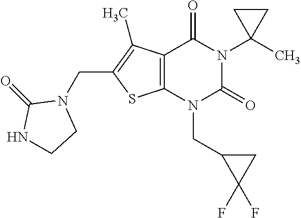 [SMILES: Cc1c(CN2CCNC2=O)sc2c1c(=O)n(C1(C)CC1)c(=O)n2CC1CC1(F)F]

To a solution of 567 mg (1.27 mmol, 89% purity) of the compound from Ex. 117A and 265 μl (1.90 mmol) of triethylamine in 13 ml of THF were added 246 mg (1.52 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 410 mg (76% of theory) of the title compound were obtained.

¹H-NMR (400 MHz, DMSO-d₆, δ/ppm): 6.52 (s, 1H), 4.35 (s, 2H), 4.22-3.75 (m, 2H), 3.28-3.16 (m, 4H), 2.39 (s, 3H), 2.27-2.11 (m, 1H), 1.78-1.62 (m, 1H), 1.50-1.39 (m, 1H), 1.34 (s, 3H), 0.98-0.75 (m, 4H).

LC/MS (Method 2, ESIpos): Rₜ=0.82 min, m/z=425 [M+H]⁺.

### Example 64

### 1-[(2,2-Difluorocyclopropyl)methyl]-5-methyl-3-(1-methylcyclopropyl)-6-[(2-oxoimidazolidin-1-yl)methyl]thieno[2,3-d]pyrimidine-2,4(1H,3H)-dione (enantiomer 1)

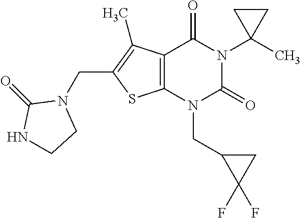 [SMILES: Cc1c(CN2CCNC2=O)sc2c1c(=O)n(C1(C)CC1)c(=O)n2CC1CC1(F)F]

398 mg of the racemic compound from Ex. 63 were dissolved in 40 ml of methanol and, in 66 portions, separated into the enantiomers by preparative SFC-HPLC on a chiral phase [column: Daicel Chiralpak OD-H, 5 μm, 250 mm×20 mm; eluent: carbon dioxide/methanol 70:30; flow rate: 150 ml/min; temperature: 40° C.; detection: 210 nm]. After concentration of the product fractions and drying of the solids under high vacuum, 192 mg (96% of theory) of enantiomer 1 were obtained (>99% ee, chiral analytical HPLC).

¹H-NMR (400 MHz, DMSO-d₆, δ/ppm): 6.52 (s, 1H), 4.35 (s, 2H), 4.21-3.76 (m, 2H), 3.28-3.17 (m, 4H), 2.39 (s, 3H), 2.26-2.10 (m, 1H), 1.78-1.62 (m, 1H), 1.50-1.40 (m, 1H), 1.34 (s, 3H), 0.98-0.75 (m, 4H).

Chiral analytical HPLC [column: Daicel Chiralpak OD-3, 3 μm, 50 mm×4.6 mm; eluent: carbon dioxide/methanol 95:5→40:60→95:5; flow rate: 3 ml/min; temperature: 40° C.; detection: 220 nm]: Rₜ=2.98 min.

### Example 65

### 1-[(2,2-Difluorocyclopropyl)methyl]-5-methyl-3-(1-methylcyclopropyl)-6-[(2-oxoimidazolidin-1-yl)methyl]thieno[2,3-d]pyrimidine-2,4(1H,3H)-dione (enantiomer 2)

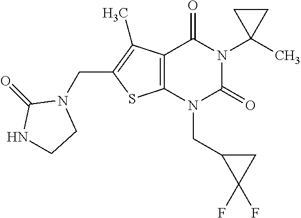 [SMILES: Cc1c(CN2CCNC2=O)sc2c1c(=O)n(C1(C)CC1)c(=O)n2CC1CC1(F)F]

398 mg of the racemic compound from Ex. 63 were dissolved in 40 ml of methanol and, in 66 portions, separated into the enantiomers by preparative SFC-HPLC on a chiral phase [column: Daicel Chiralpak OD-H, 5 μm, 250 mm×20 mm; eluent: carbon dioxide/methanol 70:30; flow rate: 150 ml/min; temperature: 40° C.; detection: 210 nm]. After concentration of the product fractions and drying of the solids under high vacuum, 156 mg (78% of theory) of enantiomer 2 were obtained (99% ee, chiral analytical HPLC).

¹H-NMR (400 MHz, DMSO-d₆, δ/ppm): 6.52 (s, 1H), 4.35 (s, 2H), 4.22-3.75 (m, 2H), 3.28-3.16 (m, 4H), 2.39 (s, 3H), 2.27-2.08 (m, 1H), 1.78-1.61 (m, 1H), 1.50-1.40 (m, 1H), 1.34 (s, 3H), 0.97-0.74 (m, 4H).

Chiral analytical HPLC [column: Daicel Chiralpak OD-3, 3 μm, 50 mm×4.6 mm; eluent: carbon dioxide/methanol 95:5→40:60→95:5; flow rate: 3 ml/min; temperature: 40° C.; detection: 220 nm]: Rₜ=3.31 min.

### Example 66

### 1-[(2,2-Difluorocyclopropyl)methyl]-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione (racemate)

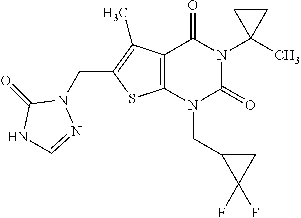 [SMILES: Cc1c(Cn2nc[nH]c2=O)sc2c1c(=O)n(C1(C)CC1)c(=O)n2CC1CC1(F)F]

Analogously to the method described in Ex. 2, 668 mg (1.20 mmol, 92% purity) of the compound from Ex. 261A were used to prepare 357 mg (70% of theory) of the title compound.

¹H-NMR (400 MHz, DMSO-d₆, δ/ppm): 11.59 (br. s, 1H), 7.83 (s, 1H), 4.93 (s, 2H), 4.22-3.74 (m, 2H), 2.45 (s, 3H), 2.24-2.08 (m, 1H), 1.77-1.60 (m, 1H), 1.49-1.39 (m, 1H), 1.33 (s, 3H), 0.98-0.73 (m, 4H).

LC/MS (Method 1, ESIpos): Rₜ=1.42 min, m/z=424.12 [M+H]⁺.

### Example 67

### 1-[(2,2-Difluorocyclopropyl)methyl]-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione (enantiomer 1)

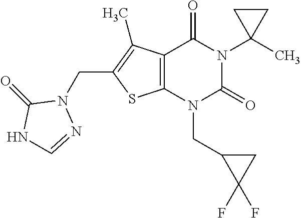 [SMILES: Cc1c(Cn2nc[nH]c2=O)sc2c1c(=O)n(C1(C)CC1)c(=O)n2CC1CC1(F)F]

349 mg of the racemic compound from Ex. 66 were dissolved in 27 ml of methanol and, in 33 portions, separated into the enantiomers by preparative SFC-HPLC on a chiral phase [column: Daicel Chiralpak OD-H, 5 μm, 250 mm×20 mm; eluent: carbon dioxide/methanol 72:28; flow rate: 70 ml/min; temperature: 40° C.; detection: 210 nm]. After concentration of the product fractions and drying of the solids under high vacuum, 151 mg (86% of theory) of enantiomer 1 were obtained (>99% ee, chiral analytical HPLC).

¹H-NMR (400 MHz, DMSO-d₆, δ/ppm): 11.58 (br. s, 1H), 7.83 (s, 1H), 4.93 (s, 2H), 4.23-3.73 (m, 2H), 2.45 (s, 3H), 2.24-2.06 (m, 1H), 1.77-1.61 (m, 1H), 1.49-1.38 (m, 1H), 1.33 (s, 3H), 0.99-0.68 (m, 4H).

Chiral analytical HPLC [column: Daicel Chiralpak OD-3, 3 μm, 50 mm×4.6 mm; eluent: carbon dioxide/methanol 70:30; flow rate: 3 ml/min; temperature: 40° C.; detection: 210 nm]: Rₜ=0.97 min.

### Example 68

### 1-[(2,2-Difluorocyclopropyl)methyl]-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione (enantiomer 2)

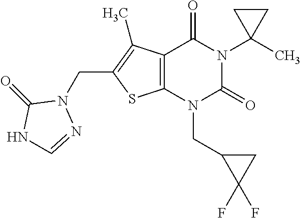 [SMILES: Cc1c(Cn2nc[nH]c2=O)sc2c1c(=O)n(C1(C)CC1)c(=O)n2CC1CC1(F)F]

349 mg of the racemic compound from Ex. 66 were dissolved in 27 ml of methanol and, in 33 portions, separated into the enantiomers by preparative SFC-HPLC on a chiral phase [column: Daicel Chiralpak OD-H, 5 μm, 250 mm×20 mm; eluent: carbon dioxide/methanol 72:28; flow rate: 70 ml/min; temperature: 40° C.; detection: 210 nm]. After concentration of the product fractions and drying of the solids under high vacuum, 140 mg (80% of theory) of enantiomer 2 were obtained (>99% ee, chiral analytical HPLC).

¹H-NMR (400 MHz, DMSO-d₆, δ/ppm): 11.58 (br. s, 1H), 7.83 (s, 1H), 4.93 (s, 2H), 4.23-3.73 (m, 2H), 2.45 (s, 3H), 2.23-2.08 (m, 1H), 1.76-1.62 (m, 1H), 1.49-1.39 (m, 1H), 1.33 (s, 3H), 0.99-0.73 (m, 4H).

Chiral analytical HPLC [column: Daicel Chiralpak OD-3, 3 μm, 50 mm×4.6 mm; eluent: carbon dioxide/methanol 70:30; flow rate: 3 ml/min; temperature: 40° C.; detection: 210 nm]: Rₜ=1.03 min.

### Example 69

### [5-Methyl-3-(1-methylcyclopropyl)-2,4-dioxo-6-[(5-oxo-4,5-dihydro-1H-1,2,4-triazol-1-yl)methyl]-3,4-dihydrothieno[2,3-d]pyrimidin-1(2H)-yl]acetonitrile

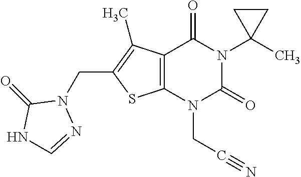 [SMILES: Cc1c(Cn2nc[nH]c2=O)sc2c1c(=O)n(C1(C)CC1)c(=O)n2CC#N]

Analogously to the method described in Ex. 2, 123 mg (0.245 mmol, 92% purity) of the compound from Ex. 262A were used to prepare 56 mg (62% of theory) of the title compound.

¹H-NMR (400 MHz, DMSO-d₆, δ/ppm): 11.61 (br. s, 1H), 7.84 (s, 1H), 5.07 (br. d, 2H), 4.96 (s, 2H), 2.46 (s, 3H), 1.34 (s, 3H), 0.97-0.79 (m, 4H).

LC/MS (Method 1, ESIpos): Rₜ=1.14 min, m/z=373.11 [M+H]⁺.

### Example 70

### 1-(2-Methoxyethyl)-5-methyl-3-(1-methylcyclopropyl)-6-[(2-oxoimidazolidin-1-yl)methyl]thieno[2,3-d]pyrimidine-2,4(1H,3H)-dione

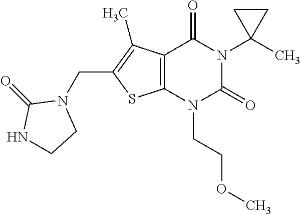 [SMILES: COCCn1c(=O)n(C2(C)CC2)c(=O)c2c(C)c(CN3CCNC3=O)sc21]

To a solution of 19.1 g (42.2 mmol, 81% purity) of the compound from Ex. 118A and 8.8 ml (63.3 mmol) of triethylamine in 420 ml of THF were added 8.21 g (50.7 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was stirred first at RT in diethyl ether/ethyl acetate (10:1) and then in hot ethyl acetate. After being cooled, the solids were filtered off with suction and dried under high vacuum. 7.18 g (43% of theory) of the title compound were obtained.

¹H-NMR (400 MHz, DMSO-d₆, δ/ppm): 6.51 (s, 1H), 4.33 (s, 2H), 4.12-3.83 (m, 2H), 3.61 (t, 2H), 3.27-3.15 (m, 4H), 3.24 (s, 3H), 2.37 (s, 3H), 1.33 (s, 3H), 0.99-0.73 (m, 4H).

LC/MS (Method 1, ESIpos): Rₜ=1.22 min, m/z=393.16 [M+H]⁺.

### Example 71

### [1-{[1-(2-Methoxyethyl)-5-methyl-3-(1-methylcyclopropyl)-2,4-dioxo-1,2,3,4-tetrahydrothieno[2,3-d]pyrimidin-6-yl]methyl}imidazolidine-2-ylidene]cyanamide

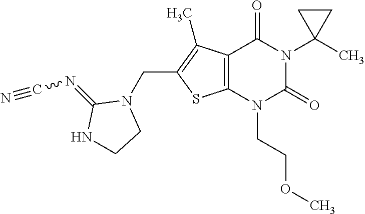 [SMILES: COCCn1c(=O)n(C2(C)CC2)c(=O)c2c(C)c(CN3CCNC3=NC#N)sc21]

310 mg (0.38 mmol, 44% purity) of the compound from Example 118A were dissolved in 14 ml of DMF, and 103 mg (0.76 mmol) of potassium carbonate were added. The mixture was stirred at RT for 15 min, then 82 mg (0.57 mmol) of dimethyl N-cyanodithioiminocarbonate were added, and the reaction mixture was stirred in a microwave oven (Biotage Initiator with dynamic control of irradiation power) at 80° C. for 1.5 h. Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 22 mg (14% of theory) of the title compound.

¹H-NMR (400 MHz, CD₃OD, δ/ppm): 4.53 (s, 2H), 4.21-4.12 (m, 1H), 4.09-3.99 (m, 1H), 3.71 (t, 2H), 3.53 (s, 4H), 3.33 (s, 3H), 2.48 (s, 3H), 1.41 (s, 3H), 1.02-0.95 (m, 1H), 0.95-0.88 (m, 3H).

LC/MS (Method 2, ESIpos): Rₜ=0.76 min, m/z=417 [M+H]⁺.

### Example 72

### Methyl [1-{[1-(2-methoxyethyl)-5-methyl-3-(1-methylcyclopropyl)-2,4-dioxo-1,2,3,4-tetrahydrothieno[2,3-d]pyrimidin-6-yl]methyl}imidazolidine-2-ylidene]carbamate

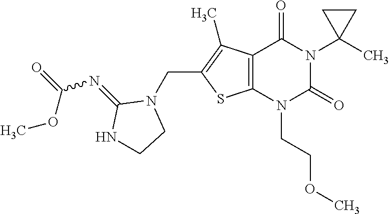

250 mg (0.30 mmol, 44% purity) of the compound from Ex. 118A and 84 μl (0.61 mmol) of triethylamine were dissolved in 14 ml of dichloromethane, and 47 mg (0.30 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 59 mg (42% of theory) of the title compound.

¹H-NMR (400 MHz, CD₃OD, δ/ppm): 4.78 (s, 2H), 4.23-4.15 (m, 1H), 4.10-3.98 (m, 1H), 3.91 (s, 3H), 3.79 (t, 2H), 3.74-3.68 (m, 2H), 3.65 (t, 2H), 3.31 (s, 3H), 2.47 (s, 3H), 1.41 (s, 3H), 1.03-0.87 (m, 4H).

LC/MS (Method 2,ESIpos): Rₜ=0.65 min, m/z=450 [M+H]⁺.

### Example 73

### 6-[(2,3-Dioxopiperazin-1-yl)methyl]-1-(2-methoxyethyl)-5-methyl-3-(1-methylcyclopropyl)thieno[2,3-d]pyrimidine-2,4(1H,3H)-dione

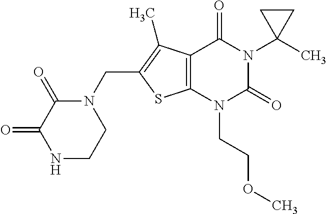 [SMILES: COCCn1c(=O)n(C2(C)CC2)c(=O)c2c(C)c(CN3CCNC(=O)C3=O)sc21]

300 mg (0.36 mmol, 44% purity) of the compound from Example 118A were dissolved in 15 ml of ethanol, and 99 mg (0.67 mmol) of diethyl oxalate were added. The mixture was stirred at 80° C. overnight. Thereafter, the reaction solution was concentrated on a rotary evaporator. The residue obtained was purified by preparative HPLC (Method 13). Concentration of the product fractions and drying under high vacuum gave 61 mg (39% of theory) of the title compound.

¹H-NMR (400 MHz, CD₃OD, δ/ppm): 4.80 (s, 2H), 4.20-4.10 (m, 1H), 4.05-3.98 (m, 1H), 3.70 (dd, 2H), 3.58 (dd, 2H), 3.45 (dd, 2H), 3.31 (br. s, 2H), 2.49 (s, 3H), 1.41 (s, 3H), 1.03-0.97 (m, 1H), 0.95-0.70 (m, 3H).

LC/MS (Method 2, ESIpos): Rₜ=0.63 min, m/z=421 [M+H]⁺.

### Example 74

### 1-(2-Methoxyethyl)-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione

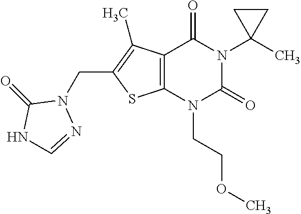 [SMILES: COCCn1c(=O)n(C2(C)CC2)c(=O)c2c(C)c(Cn3nc[nH]c3=O)sc21]

500 mg (0.87 mmol, 83% purity) of the compound from Ex. 263A were initially charged in 21.3 ml of trimethyl orthoformate and 21.3 ml of methanol, and 2.2 ml (8.68 mmol) of a 4 M solution of hydrogen chloride in dioxane were added. Since the conversion was still incomplete after stirring at RT for 18 h, a further 1.1 ml of 4 M hydrogen chloride in dioxane was added and the mixture was stirred for a further 2.5 h. Thereafter, another 1.1 ml of 4 M hydrogen chloride in dioxane was added, and the mixture was stirred for a further 2.5 h. Then water was added to the reaction mixture, which was extracted with ethyl acetate. The organic phase was concentrated and the residue was purified by means of preparative HPLC (Method 13). The product fractions were combined and concentrated, and the residue was dried under high vacuum. 125 mg (36% of theory) of the title compound were obtained.

¹H-NMR (400 MHz, CD3OD, δ/ppm): 7.72 (s, 1H), 5.07 (s, 2H), 4.25-4.10 (m, 2H), 2.75-2.67 (m, 2H), 2.56 (s, 3H), 1.43 (s, 3H), 1.03-0.90 (m, 4H).

LC/MS (Method 2, ESIpos): Rₜ=0.65 min, m/z=392 [M+H]⁺.

### Example 75

### 5-Methyl-3-(1-methylcyclopropyl)-6-[(2-oxoimidazolidin-1-yl)methyl]-1-[2-(trifluormethoxy)ethyl]thieno[2,3-d]pyrimidine-2,4(1H,3H)-dione

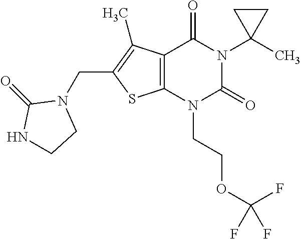 [SMILES: Cc1c(CN2CCNC2=O)sc2c1c(=O)n(C1(C)CC1)c(=O)n2CCOC(F)(F)F]

To a solution of 355 mg (0.785 mmol, 93% purity) of the compound from Ex. 119A and 164 μl (1.18 mmol) of triethylamine in 8 ml of THF were added 153 mg (0.942 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 223 mg (63% of theory) of the title compound were obtained.

¹H-NMR (400 MHz, DMSO-d₆, δ/ppm): 6.52 (s, 1H), 4.38 (br. t, 2H), 4.34 (s, 2H), 4.27-4.00 (m, 2H), 3.26-3.15 (m, 4H), 2.38 (s, 3H), 1.33 (s, 3H), 1.00-0.73 (m, 4H).

LC/MS (Method 1, ESIpos): Rₜ=1.57 min, m/z=447.13 [M+H]⁺.

### Example 76

### 5-Methyl-3-(1-methylcyclopropyl)-6-[(5-oxo-4,5-dihydro-1H-1,2,4-triazol-1-yl)methyl]-1-[2-(trifluormethoxy)ethyl]thieno[2,3-d]pyrimidine-2,4(1H,3H)-dione

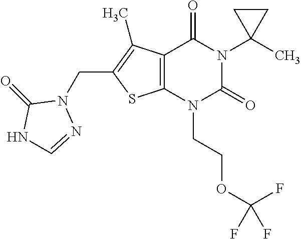 [SMILES: Cc1c(Cn2nc[nH]c2=O)sc2c1c(=O)n(C1(C)CC1)c(=O)n2CCOC(F)(F)F]

234 mg (0.411 mmol, 94% purity) of the compound from Ex. 264A were dissolved in a mixture of 14 ml each of methanol and trimethyl orthoformate, and 1 ml (4.11 mmol) of a 4 M solution of hydrogen chloride in dioxane were added at RT. After 2 days, the reaction mixture was concentrated and the residue was taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 125 mg (68% of theory) of the title compound were obtained.

¹H-NMR (400 MHz, DMSO-d₆, δ/ppm): 11.58 (br. s, 1H), 7.82 (s, 1H), 4.92 (s, 2H), 4.37 (br. t, 2H), 4.28-3.99 (m, 2H), 2.44 (s, 3H), 1.33 (s, 3H), 0.97-0.72 (m, 4H).

LC/MS (Method 1, ESIpos): Rₜ=1.46 min, m/z=446.11 [M+H]⁺.

### Example 77

### 1-(2-Ethoxyethyl)-5-methyl-3-(1-methylcyclopropyl)-6-[(2-oxoimidazolidin-1-yl)methyl]thieno[2,3-d]pyrimidine-2,4(1H,3H)-dione

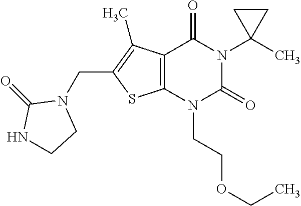 [SMILES: CCOCCn1c(=O)n(C2(C)CC2)c(=O)c2c(C)c(CN3CCNC3=O)sc21]

Analogously to the method described in Ex. 3, 240 mg (0.536 mmol, 85% purity) of the compound from Ex. 120A and 104 mg (0.643 mmol) of CDI were used to prepare 122 mg (55% of theory) of the title compound.

¹H-NMR (400 MHz, DMSO-d₆, δ/ppm): 6.51 (s, 1H), 4.33 (s, 2H), 4.12-3.82 (m, 2H), 3.64 (t, 2H), 3.43 (q, 2H), 3.26-3.15 (m, 4H), 2.38 (s, 3H), 1.33 (s, 3H), 1.04 (t, 3H), 0.96-0.74 (m, 4H).

LC/MS (Method 1, ESIpos): Rₜ=1.38 min, m/z=407.17 [M+H]⁺.

### Example 78

### 1-(2-Ethoxyethyl)-5-methyl-3-(1-methylcyclopropyl)-6-[(5-oxo-4,5-dihydro-1H-1,2,4-triazol-1-yl)methyl]thieno[2,3-d]pyrimidine-2,4(1H,3H)-dione

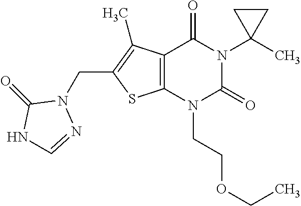 [SMILES: CCOCCn1c(=O)n(C2(C)CC2)c(=O)c2c(C)c(Cn3nc[nH]c3=O)sc21]

Analogously to the method described in Ex. 2, 155 mg (0.313 mmol) of the compound from Ex. 265A were used to prepare 73 mg (57% of theory) of the title compound. The product here, after isolation by means of preparative HPLC, was stirred again with acetonitrile at RT.

¹H-NMR (400 MHz, DMSO-d₆, δ/ppm): 11.58 (br. s, 1H), 7.82 (s, 1H), 4.91 (s, 2H), 4.11-3.80 (m, 2H), 3.62 (br. t, 2H), 3.42 (q, 2H), 2.44 (s, 3H), 1.33 (s, 3H), 1.02 (t, 3H), 0.95-0.74 (m, 4H).

LC/MS (Method 1, ESIpos): Rₜ=1.27 min, m/z=406.15 [M+H]⁺.

### Example 79

### 1-(2-Isopropoxyethyl)-5-methyl-3-(1-methylcyclopropyl)-6-[(2-oxoimidazolidin-1-yl)methyl]thieno[2,3-d]pyrimidine-2,4(1H,3H)-dione

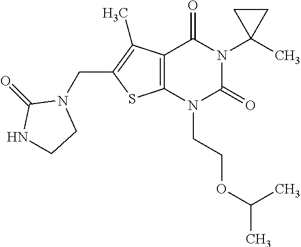 [SMILES: Cc1c(CN2CCNC2=O)sc2c1c(=O)n(C1(C)CC1)c(=O)n2CCOC(C)C]

Analogously to the method described in Ex. 3, 250 mg (0.570 mmol, 90% purity) of the compound from Ex. 121A and 111 mg (0.684 mmol) of CDI were used to prepare 153 mg (63% of theory) of the title compound.

¹H-NMR (400 MHz, DMSO-d₆, δ/ppm): 6.51 (s, 1H), 4.33 (s, 2H), 4.06-3.80 (m, 2H), 3.63 (t, 2H), 3.54 (sept, 1H), 3.26-3.15 (m, 4H), 2.37 (s, 3H), 1.33 (s, 3H), 1.00 (d, 6H), 0.96-0.74 (m, 4H).

LC/MS (Method 1, ESIpos): Rₜ=1.50 min, m/z=421.19 [M+H]⁺.

### Example 80

### 1-(2-Isopropoxyethyl)-5-methyl-3-(1-methylcyclopropyl)-6-[(5-oxo-4,5-dihydro-H-1,2,4-triazol-1-yl)methyl]thieno[2,3-d]pyrimidine-2,4(1H,3H)-dione

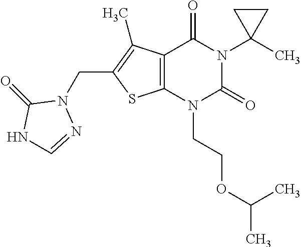 [SMILES: Cc1c(Cn2nc[nH]c2=O)sc2c1c(=O)n(C1(C)CC1)c(=O)n2CCOC(C)C]

255 mg (0.500 mmol) of the compound from Ex. 266A were dissolved in a mixture of 16 ml each of methanol and trimethyl orthoformate, and 1.3 ml (5.00 mmol) of a 4 M solution of hydrogen chloride in dioxane were added at RT. After 2 days, the reaction mixture was concentrated and the residue was taken up in ethyl acetate. The mixture was washed successively with water and saturated sodium chloride solution. After drying over anhydrous magnesium sulfate, the mixture was filtered and concentrated again. The residue that remained was stirred with a little acetonitrile at RT. The solids were filtered off with suction and dried under high vacuum. The filtrate was concentrated, and the residue was purified by means of preparative HPLC (Method 11). After concentration of the product fractions, drying under high vacuum and combination with the solids isolated beforehand, a total of 138 mg (65% of theory) of the title compound were obtained.

¹H-NMR (400 MHz, DMSO-d₆, δ/ppm): 11.57 (br. s, 1H), 7.82 (s, 1H), 4.91 (s, 2H), 4.08-3.78 (m, 2H), 3.62 (t, 2H), 3.53 (sept, 1H), 2.44 (s, 3H), 1.33 (s, 3H), 0.99 (d, 6H), 0.95-0.72 (m, 4H).

LC/MS (Method 1, ESIpos): Rₜ=1.39 min, m/z=420.17 [M+H]⁺.

### Example 81

### 5-Methyl-3-(1-methylcyclopropyl)-6-[(2-oxoimidazolidin-1-yl)methyl]-1-[(2R)-tetrahydrofuran-2-ylmethyl]thieno[2,3-d]pyrimidine-2,4(1H,3H)-dione

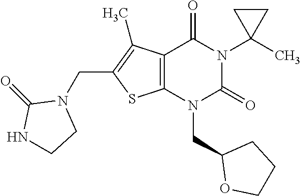 [SMILES: Cc1c(CN2CCNC2=O)sc2c1c(=O)n(C1(C)CC1)c(=O)n2C[C@H]1CCCO1]

To a solution of 220 mg (0.532 mmol, 95% purity) of the compound from Ex. 122A and 111 μl (0.799 mmol) of triethylamine in 6 ml of THF were added 104 mg (0.639 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 125 mg (56% of theory) of the title compound were obtained.

¹H-NMR (400 MHz, DMSO-d₆, δ/ppm): 6.50 (s, 1H), 4.33 (s, 2H), 4.25-4.14 (m, 1H), 4.13-3.85 (m, 1H), 3.82-3.48 (m, 3H), 3.27-3.15 (m, 4H), 2.37 (s, 3H), 2.03-1.74 (m, 3H), 1.70-1.58 (m, 1H), 1.33 (s, 3H), 0.98-0.72 (m, 4H).

LC/MS (Method 1, ESIpos): Rₜ=1.36 min, m/z=419.17 [M+H]⁺.

### Example 82

### 5-Methyl-3-(1-methylcyclopropyl)-6-[(2-oxoimidazolidin-1-yl)methyl]-1-[(2S)-tetrahydrofuran-2-ylmethyl]thieno[2,3-d]pyrimidine-2,4(1H,3H)-dione

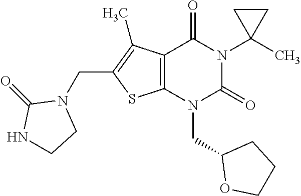 [SMILES: Cc1c(CN2CCNC2=O)sc2c1c(=O)n(C1(C)CC1)c(=O)n2C[C@@H]1CCCO1]

Analogously to the method described in Ex. 3, 308 mg (0.706 mmol, 90% purity) of the compound from Ex. 123A were used to prepare 188 mg (63% of theory) of the title compound.

¹H-NMR (400 MHz, DMSO-d₆, δ/ppm): 6.50 (s, 1H), 4.33 (s, 2H), 4.25-4.14 (m, 1H), 4.13-3.86 (m, 1H), 3.81-3.50 (m, 3H), 3.26-3.15 (m, 4H), 2.38 (s, 3H), 2.03-1.75 (m, 3H), 1.71-1.58 (m, 1H), 1.33 (s, 3H), 0.99-0.72 (m, 4H).

LC/MS (Method 1, ESIpos): Rₜ=1.36 min, m/z=419.17 [M+H]⁺.

### Example 83

### 5-Methyl-3-(1-methylcyclopropyl)-6-[(5-oxo-4,5-dihydro-1H-1,2,4-triazol-1-yl)methyl]-1-[(2R)-tetrahydrofuran-2-ylmethyl]thieno[2,3-d]pyrimidine-2,4(1H, 3H)-dione

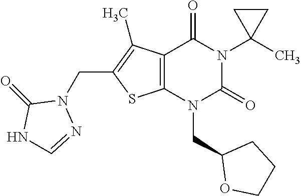 [SMILES: Cc1c(Cn2nc[nH]c2=O)sc2c1c(=O)n(C1(C)CC1)c(=O)n2C[C@H]1CCCO1]

Analogously to the method described in Ex. 2, 155 mg (0.266 mmol, 87% purity) of the compound from Ex. 267A were used to prepare 40 mg (35% of theory) of the title compound. The reaction time here was about 16 h.

¹H-NMR (400 MHz, DMSO-d₆, δ/ppm): 11.58 (br. s, 1H), 7.82 (s, 1H), 4.91 (s, 2H), 4.26-3.84 (m, 2H), 3.80-3.46 (m, 3H), 2.43 (s, 3H), 2.02-1.74 (m, 3H), 1.71-1.56 (m, 1H), 1.33 (s, 3H), 0.98-0.70 (m, 4H).

LC/MS (Method 2, ESIpos): Rₜ=0.69 min, m/z=418 [M+H]⁺.

### Example 84

### 5-Methyl-3-(1-methylcyclopropyl)-6-[(5-oxo-4,5-dihydro-1H-1,2,4-triazol-1-yl)methyl]-1-[(2S)-tetrahydrofuran-2-ylmethyl]thieno[2,3-d]pyrimidine-2,4(1H,3H)-dione

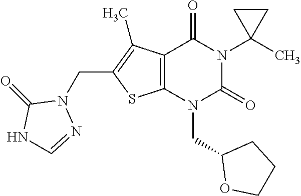 [SMILES: Cc1c(Cn2nc[nH]c2=O)sc2c1c(=O)n(C1(C)CC1)c(=O)n2C[C@@H]1CCCO1]

Analogously to the method described in Ex. 2, 295 mg (0.581 mmol) of the compound from Ex. 268A were used to prepare 90 mg (37% of theory) of the title compound. The reaction time here was about 16 h.

¹H-NMR (400 MHz, DMSO-d₆, δ/ppm): 11.57 (s, 1H), 7.83 (s, 1H), 4.91 (s, 2H), 4.28-3.86 (m, 2H), 3.80-3.46 (m, 3H), 2.43 (s, 3H), 2.03-1.75 (m, 3H), 1.70-1.53 (m, 1H), 1.33 (s, 3H), 0.99-0.70 (m, 4H).

LC/MS (Method 1, ESIpos): Rₜ=1.25 min, m/z=418.15 [M+H]⁺.

### Example 85

### 1-(2-Methoxyethyl)-5-methyl-6-[(2-oxoimidazolidin-1-yl)methyl]-3-[1-(trifluoromethyl)cyclopropyl]thieno[2,3-d]pyrimidine-2,4(1H,3H)-dione

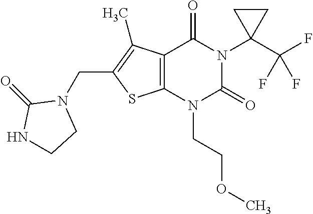 [SMILES: COCCn1c(=O)n(C2(C(F)(F)F)CC2)c(=O)c2c(C)c(CN3CCNC3=O)sc21]

Analogously to the method described in Ex. 31, 320 mg (0.723 mmol, 95% purity) of the compound from Ex. 124A and 141 mg (0.868 mmol) of CDI were used to prepare 171 mg (52% of theory) of the title compound.

¹H-NMR (400 MHz, DMSO-d₆, δ/ppm): 6.53 (s, 1H), 4.34 (s, 2H), 4.11-3.90 (m, 2H), 3.61 (t, 2H), 3.28-3.16 (m, 4H), 3.24 (s, 3H), 2.37 (s, 3H), 1.66-1.49 (m, 2H), 1.39-1.30 (m, 2H).

LC/MS (Method 1, ESIpos): Rₜ=1.43 min, m/z=447.13 [M+H]⁺.

### Example 86

### [1-({1-(2-Methoxy ethyl)-5-methyl-2,4-dioxo-3-[1-(trifluoromethyl)cyclopropyl]-1,2,3,4-tetrahydrothieno[2,3-d]pyrimidin-6-yl}methyl)imidazolidin-2-ylidene]cyanamide

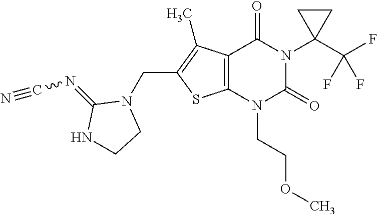 [SMILES: COCCn1c(=O)n(C2(C(F)(F)F)CC2)c(=O)c2c(C)c(CN3CCNC3=NC#N)sc21]

Analogously to the method described in Ex. 41, 320 mg (0.723 mmol, 95% purity) of the compound from Ex. 124A and 159 mg (1.09 mmol) of dimethyl N-cyanodithioiminocarbonate were used to prepare 116 mg (33% of theory) of the title compound.

¹H-NMR (400 MHz, DMSO-d₆, δ/ppm): 8.08 (s, 1H), 4.47 (s, 2H), 4.09-3.93 (m, 2H), 3.62 (t, 2H), 3.51-3.36 (m, 4H), 3.24 (s, 3H), 2.39 (s, 3H), 1.67-1.50 (m, 2H), 1.41-1.28 (m, 2H).

LC/MS (Method 1, ESIpos): Rₜ=1.54 min, m/z=471.14 [M+H]⁺.

### Example 87

### Methyl [1-({1-(2-methoxyethyl)-5-methyl-2,4-dioxo-3-[1-(trifluoromethyl)cyclopropyl]-1,2,3,4-tetrahydrothieno[2,3-d]pyrimidin-6-yl}methyl)imidazolidin-2-ylidene]carbamate

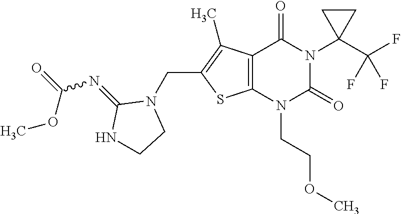 [SMILES: COCCn1c(=O)n(C2(C(F)(F)F)CC2)c(=O)c2c(C)c(CN3CCNC3=NC(=O)OC)sc21]

320 mg (0.723 mmol, 95% purity) of the compound from Ex. 124A and 202 μl (1.45 mmol) of triethylamine were dissolved in 10 ml of dichloromethane, and a solution of 226 mg (1.45 mmol) of methyl (dichloromethylene)carbamate in 5 ml of dichloromethane was added dropwise. After the reaction mixture had been stirred at RT for about 18 h, it was diluted with water and extracted with ethyl acetate. The organic extract was washed with saturated sodium chloride solution, dried over anhydrous magnesium sulfate, filtered and concentrated. 115 mg (30% of theory) of the title compound were isolated from the residue by means of preparative HPLC (Method 11).

¹H-NMR (400 MHz, DMSO-d₆, δ/ppm): 8.02 (s, 1H), 4.55 (s, 2H), 4.08-3.91 (m, 2H), 3.61 (t, 2H), 3.53 (s, 3H), 3.49-3.42 (m, 2H), 3.39-3.30 (m, 2H, partially concealed by water signal), 3.23 (s, 3H), 2.40 (s, 3H), 1.66-1.51 (m, 2H), 1.41-1.27 (m, 2H).

LC/MS (Method 1, ESIpos): Rₜ=1.35 min, m/z=504.15 [M+H]⁺.

### Example 88

### 6-[(2,3-Dioxopiperazin-1-yl)methyl]-1-(2-methoxyethyl)-5-methyl-3-[1-(trifluoromethyl)cyclopropyl]thieno[2,3-d]pyrimidine-2,4(1H,3H)-dione

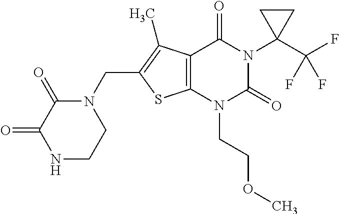 [SMILES: COCCn1c(=O)n(C2(C(F)(F)F)CC2)c(=O)c2c(C)c(CN3CCNC(=O)C3=O)sc21]

To a solution of 320 mg (0.723 mmol, 95% purity) of the compound from Ex. 124A in 15 ml of ethanol were added 185 μl (1.34 mmol) of diethyl oxalate, and the mixture was stirred at 80° C. for about 16 h. Thereafter, the reaction mixture was concentrated and the residue was taken up in ethyl acetate. The organic phase was washed successively with saturated sodium hydrogencarbonate solution, water and saturated sodium chloride solution. After drying over anhydrous magnesium sulfate, the mixture was filtered and concentrated. 133 mg (37% of theory) of the title compound were isolated from the residue that remained by means of preparative HPLC (Method 11).

¹H-NMR (400 MHz, DMSO-d₆, δ/ppm): 8.62 (br. s, 1H), 4.69 (d, 2H), 4.09-3.93 (m, 2H), 3.61 (t, 2H), 3.54-3.42 (m, 2H), 3.34-3.28 (m, 2H, almost entirely concealed by water signal), 3.24 (s, 3H), 2.41 (s, 3H), 1.66-1.51 (m, 2H), 1.40-1.28 (m, 2H).

LC/MS (Method 1, ESIneg): Rₜ=1.28 min, m/z=519.12 [M−H]⁻.

### Example 89

### 1-(2-Methoxyethyl)-5-methyl-6-[(5-oxo-4,5-dihydro-1H-1,2,4-triazol-1-yl)methyl]-3-[1-(trifluoromethyl)cyclopropyl]thieno[2,3-d]pyrimidine-2,4(1H,3H)-dione

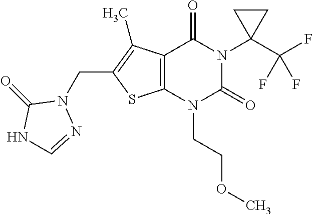 [SMILES: COCCn1c(=O)n(C2(C(F)(F)F)CC2)c(=O)c2c(C)c(Cn3nc[nH]c3=O)sc21]

Analogously to the method described in Ex. 50, 490 mg (0.796 mmol, 87% purity) of the compound from Ex. 269A were used to prepare 75 mg (21% of theory) of the title compound.

¹H-NMR (400 MHz, DMSO-d₆, δ/ppm): 11.59 (br. s, 1H), 7.83 (s, 1H), 4.93 (s, 2H), 4.08-3.89 (m, 2H), 3.60 (t, 2H), 3.23 (s, 3H), 2.43 (s, 3H), 1.66-1.48 (m, 2H), 1.40-1.26 (m, 2H).

LC/MS (Method 1, ESIpos): Rₜ=1.33 min, m/z=446.11 [M+H]⁺.

### Example 90

### 1-(3-Fluoropropyl)-5-methyl-3-(2-methylcyclopropyl)-6-[(2-oxoimidazolidin-1-yl)methyl]thieno[2,3-d]pyrimidine-2,4(1H,3H)-dione (trans racemate)

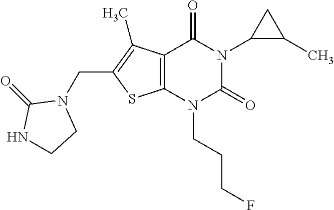 [SMILES: Cc1c(CN2CCNC2=O)sc2c1c(=O)n(C1CC1C)c(=O)n2CCCF]

510 mg (0.941 mmol) of the compound from Example 125A were dissolved in 40 ml of dioxane, and 236 mg (1.412 mmol) of CDI were added. The mixture was stirred at RT for 19 h. The reaction solution was then concentrated on a rotary evaporator. The residue was dissolved in 12 ml of DMSO and this solution was purified by means of preparative HPLC (Method 14). Combination of the product fractions and freeze-drying gave 228 mg (71% of theory) of the title compound.

¹H-NMR (400 MHz, DMSO-d₆, 8/ppm): 6.53 (s, 1H), 4.58 (t, 1H), 4.46 (t, 1H), 4.34 (s, 2H), 3.99-3.86 (m, 2H), 3.27-3.16 (m, 4H), 2.37 (s, 3H), 2.25-2.19 (m, 1H), 2.12-2.03 (m, 1H), 2.03-1.95 (m, 1H), 1.14 (d, 3H), 1.04-0.92 (m, 1H), 0.86-0.78 (m, 2H).

LC/MS (Method 4, ESIpos): Rₜ=0.96 min, m/z=395 [M+H]⁺.

### Example 91

### 1-(3-Fluoropropyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

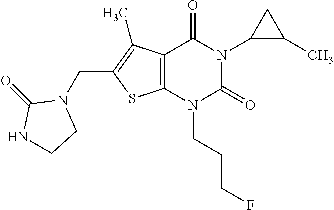 [SMILES: Cc1c(CN2CCNC2=O)sc2c1c(=O)n(C1CC1C)c(=O)n2CCCF]

228 mg of the racemic compound from Ex. 90 were dissolved in 8 ml of an ethanol/methanol mixture (1:1) and separated into the enantiomers by means of preparative HPLC on a chiral phase [column: Daicel Chiralpak AD-H, 5 μm, 250 mm×30 mm; eluent A: methanol+0.1% diethylamine (99%), eluent B: ethanol+0.1% diethylamine (99%), isocratic 50% A+50% B; flow rate: 60 ml/min; detection: 254 nm]. The product fractions were concentrated on a rotary evaporator, admixed with tert-butanol and freeze-dried. 88 mg (77% of theory) of the title compound (enantiomer 1) and 83 mg (72% of theory) of enantiomer 2 (see Ex. 92) were obtained.

¹H-NMR (400 MHz, DMSO-d₆, δ/ppm): 6.53 (s, 1H), 4.58 (t, 1H), 4.46 (t, 1H), 4.34 (s, 2H), 4.00-3.86 (m, 2H), 3.27-3.16 (m, 4H), 2.37 (s, 3H), 2.26-2.19 (m, 1H), 2.12-2.03 (m, 1H), 2.03-1.95 (m, 1H), 1.14 (d, 3H), 1.04-0.92 (m, 1H), 0.86-0.78 (m, 2H).

Chiral analytical HPLC [column: Daicel Chiralpak AD-H, 3 μm, 100 mm×4.6 mm; eluent A: methanol+0.1% diethylamine (99%), eluent B: ethanol, isocratic 50% A+50% B; flow rate: 1.4 ml/min; temperature: 25° C.; detection: 254 nm]: Rₜ=2.43 min.

### Example 92

### 1-(3-Fluoropropyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

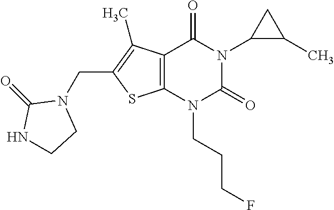 [SMILES: Cc1c(CN2CCNC2=O)sc2c1c(=O)n(C1CC1C)c(=O)n2CCCF]

The title compound (83 mg) was obtained as the second enantiomer in the preparative HPLC separation of the racemate from Ex. 90 (see Ex. 91).

¹H-NMR (400 MHz, DMSO-d₆, δ/ppm): 6.53 (s, 1H), 4.58 (t, 1H), 4.46 (t, 1H), 4.34 (s, 2H), 4.00-3.86 (m, 2H), 3.27-3.15 (m, 4H), 2.37 (s, 3H), 2.26-2.18 (m, 1H), 2.11-2.03 (m, 1H), 2.03-1.96 (m, 1H), 1.14 (d, 3H), 1.04-0.92 (m, 1H), 0.86-0.78 (m, 2H).

Chiral analytical HPLC [column: Daicel Chiralpak AD-H, 3 μm, 100 mm×4.6 mm; eluent A: methanol+0.1% diethylamine (99%), eluent B: ethanol, isocratic 50% A+50% B; flow rate: 1.4 ml/min; temperature: 25° C.; detection: Rₜ=3.05 min.

### Example 93

### 5-Methyl-3-(2-methylcyclopropyl)-6-[(2-oxoimidazolidin-1-yl)methyl]-1-(3,3,3-trifluoropropyl)thieno[2,3-d]pyrimidine-2,4(1H,3H)-dione (trans racemate)

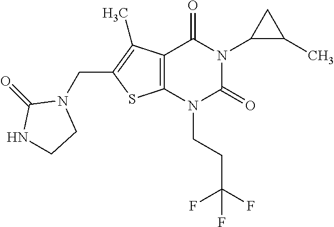 [SMILES: Cc1c(CN2CCNC2=O)sc2c1c(=O)n(C1CC1C)c(=O)n2CCC(F)(F)F]

610 mg (1.237 mmol) of the compound from Example 126A were dissolved in 50 ml of dioxane, and 310 mg (1.855 mmol) of CDI were added. The mixture was stirred at RT for 19 h. The reaction solution was then concentrated on a rotary evaporator. The residue was dissolved in 12 ml of DMSO and this solution was purified by means of preparative HPLC (Method 14). Combination of the product fractions and freeze-drying gave 380 mg (70% of theory) of the title compound.

¹H-NMR (400 MHz, DMSO-d₆, δ/ppm): 6.54 (s, 1H), 4.35 (s, 2H), 4.04 (dsext, 2H), 3.28-3.16 (m, 4H), 2.80-2.67 (m, 2H), 2.38 (s, 3H), 2.26-2.20 (m, 1H), 1.15 (d, 3H), 1.04-0.93 (m, 1H), 0.87-0.79 (m, 2H).

LC/MS (Method 4, ESIpos): Rₜ=1.06 min, m/z=431 [M+H]⁺.

### Example 94

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (trans racemate)

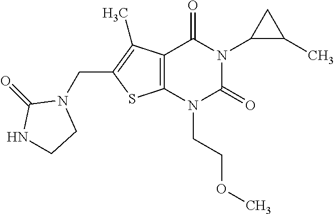 [SMILES: COCCn1c(=O)n(C2CC2C)c(=O)c2c(C)c(CN3CCNC3=O)sc21]

504 mg (1.114 mmol) of the compound from Example 127A were dissolved in 45 ml of dioxane, and 279 mg (1.671 mmol) of CDI were added. The mixture was stirred at RT for 20 h. The reaction solution was then concentrated on a rotary evaporator. The residue was dissolved in 12 ml of DMSO and this solution was purified by means of preparative HPLC (Method 14). Combination of the product fractions and freeze-drying gave 224 mg (58% of theory) of the title compound.

¹H-NMR (400 MHz, DMSO-d₆, δ/ppm): 6.53 (s, 1H), 4.33 (s, 2H), 3.97 (br. d, 2H), 3.63-3.57 (m, 2H), 3.27-3.16 (m, 7H), 2.36 (s, 3H), 2.26-2.20 (m, 1H), 1.14 (d, 3H), 1.03-0.93 (m, 1H), 0.86-0.79 (m, 2H).

LC/MS (Method 4, ESIpos): Rₜ=0.92 min, m/z=393 [M+H]⁺.

### Example 95

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

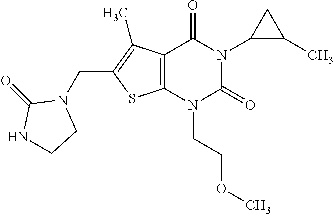 [SMILES: COCCn1c(=O)n(C2CC2C)c(=O)c2c(C)c(CN3CCNC3=O)sc21]

224 mg of the racemic compound from Ex. 94 were dissolved in 13 ml of an ethanol/methanol mixture (1:1) and separated into the enantiomers by means of preparative HPLC on a chiral phase [column: Daicel Chiralpak AD-H, 5 μm, 250 mm×30 mm; eluent A: methanol+0.1% diethylamine (99%), eluent B: ethanol+0.1% diethylamine (99%), isocratic 50% A+50% B; flow rate: 30 ml/min; detection: 254 nm]. The product fractions were concentrated on a rotary evaporator, admixed with tert-butanol and freeze-dried. 105 mg (93% of theory) of the title compound (enantiomer 1) and 106 mg (94% of theory) of enantiomer 2 (see Ex. 96) were obtained.

¹H-NMR (400 MHz, DMSO-d₆, δ/ppm): 6.53 (s, 1H), 4.33 (s, 2H), 4.03-3.91 (m, 2H), 3.60 (t, 2H), 3.27-3.16 (m, 7H), 2.36 (s, 3H), 2.23 (dt, 1H), 1.14 (d, 3H), 1.04-0.93 (m, 1H), 0.86-0.79 (m, 2H).

Chiral analytical HPLC [column: Daicel Chiralpak AD-H, 3 μm, 100 mm×4.6 mm; eluent A: methanol+0.1% diethylamine (99%), eluent B: ethanol, isocratic 50% A+50% B; flow rate: 1.4 ml/min; temperature: 25° C.; detection: 254 nm]: Rₜ=2.68 min.

### Example 96

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

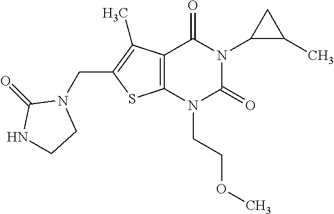 [SMILES: COCCn1c(=O)n(C2CC2C)c(=O)c2c(C)c(CN3CCNC3=O)sc21]

The title compound (106 mg) was obtained as the second enantiomer in the preparative HPLC separation of the racemate from Ex. 94 (see Ex. 95).

¹H-NMR (400 MHz, DMSO-d₆, δ/ppm): 6.53 (s, 1H), 4.33 (s, 2H), 4.03-3.91 (m, 2H), 3.60 (t, 2H), 3.27-3.16 (m, 7H), 2.36 (s, 3H), 2.23 (dt, 1H), 1.14 (d, 4H), 1.03-0.92 (m, 1H), 0.82 (dd, 2H).

Chiral analytical HPLC [column: Daicel Chiralpak AD-H, 3 μm, 100 mm×4.6 mm; eluent A: methanol+0.1% diethylamine (99%), eluent B: ethanol, isocratic 50% A+50% B; flow rate: 1.4 ml/min; temperature: 25° C.; detection: 254 nm]: Rₜ=3.60 min.

### Example 97

### 3-(2,2-Dimethylcyclopropyl)-1-(3-fluoropropyl)-5-methyl-6-[(2-oxoimidazolidin-1-yl)methyl]thieno[2,3-d]pyrimidine-2,4(1H,3H)-dione (racemate)

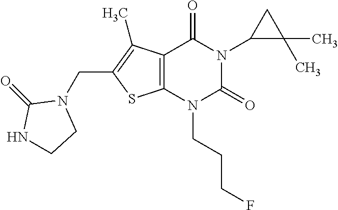 [SMILES: Cc1c(CN2CCNC2=O)sc2c1c(=O)n(C1CC1(C)C)c(=O)n2CCCF]

Analogously to Ex. 90, 869 mg (1.13 mmol, 49% purity) of the compound from Ex. 128A in 50 ml of dioxane were reacted with 279 mg (1.67 mmol) of CDI. 121 mg (26% of theory) of the title compound were obtained.

¹H-NMR (400 MHz, DMSO-d₆, δ/ppm): 6.53 (s, 1H), 4.58 (t, 1H), 4.46 (t, 1H), 4.34 (s, 2H), 4.04-3.86 (m, 2H), 3.29-3.16 (m, 4H), 2.41-2.34 (m, 4H), 2.12-2.04 (m, 1H), 2.04-1.96 (m, 1H), 1.16 (s, 3H), 1.03 (dd, 1H), 0.85 (s, 3H), 0.72 (dd, 1H).

LC/MS (Method 4, ESIpos): Rₜ=1.03 min, m/z=409 [M+H]⁺.

### Example 98

### 3-(2,2-Dimethylcyclopropyl)-1-(3-fluoropropyl)-5-methyl-6-[(2-oxoimidazolidin-1-yl)methyl]thieno[2,3-d]pyrimidine-2,4(1H,3H)-dione (enantiomer 1)

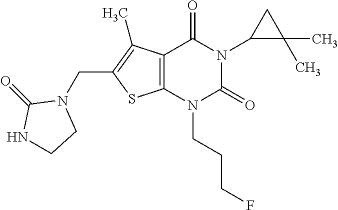 [SMILES: Cc1c(CN2CCNC2=O)sc2c1c(=O)n(C1CC1(C)C)c(=O)n2CCCF]

118 mg of the racemic compound from Ex. 97 were separated into the enantiomers by means of preparative HPLC on a chiral phase [column: Daicel Chiralpak IC, 5 μm, 250 mm×30 mm, eluent A: acetonitrile+0.1% diethylamine (99%), eluent B: ethanol, isocratic 90% A+10% B; flow rate: 50 ml/min; detection: 254 nm]. The product fractions were concentrated on a rotary evaporator, admixed with tert-butanol and freeze-dried. 48 mg (81% of theory) of the title compound (enantiomer 1) and 50 mg (84% of theory) of enantiomer 2 (see Ex. 99) were obtained.

¹H-NMR (400 MHz, DMSO-d₆, δ/ppm): 6.53 (s, 1H), 4.58 (t, 1H), 4.46 (t, 1H), 4.34 (s, 2H), 4.04-3.86 (m, 2H), 3.29-3.17 (m, 4H), 2.41-2.34 (m, 4H), 2.12-2.04 (m, 1H), 2.04-1.96 (m, 1H), 1.19-1.14 (m, 3H), 1.03 (dd, 1H), 0.85 (s, 3H), 0.75-0.69 (m, 1H).

Chiral analytical HPLC [column: Daicel Chiralpak IC, 3 μm, 100 mm×4.6 mm, eluent A: acetonitrile+0.1% diethylamine (99%), eluent B: ethanol, isocratic 90% A+10% B; flow rate: 1.4 ml/min; temperature: 25° C.; detection: 254 nm]: Rₜ=3.08 min.

### Example 99

### 3-(2,2-Dimethylcyclopropyl)-1-(3-fluoropropyl)-5-methyl-6-[(2-oxoimidazolidin-1-yl)methyl]thieno[2,3-d]pyrimidine-2,4(1H,3H)-dione (enantiomer 2)

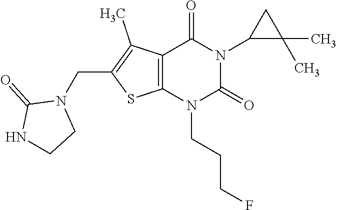 [SMILES: Cc1c(CN2CCNC2=O)sc2c1c(=O)n(C1CC1(C)C)c(=O)n2CCCF]

The title compound (50 mg) was obtained as the second enantiomer in the preparative HPLC separation of the racemate from Ex. 97 (see Ex. 98).

¹H-NMR (400 MHz, DMSO-d₆, δ/ppm): 6.53 (s, 1H), 4.58 (t, 1H), 4.46 (t, 1H), 4.34 (s, 2H), 4.04-3.86 (m, 2H), 3.29-3.17 (m, 4H), 2.40-2.36 (m, 4H), 2.12-2.04 (m, 1H), 2.04-1.96 (m, 1H), 1.16 (s, 3H), 1.06-1.00 (m, 1H), 0.85 (s, 3H), 0.75-0.69 (m, 1H).

Chiral analytical HPLC [column: Daicel Chiralpak IC, 3 μm, 100 mm×4.6 mm, eluent A: acetonitrile+0.1% diethylamine (99%), eluent B: ethanol, isocratic 90% A+10% B; flow rate: 1.4 ml/min; temperature: 25° C.; detection: 254 nm]: Rₜ=3.85 min.

### Example 100

### 3-(2,2-Dimethylcyclopropyl)-5-methyl-6-[(2-oxoimidazolidin-1-yl)methyl]-1-(3,3,3-trifluoropropyl)thieno[2,3-d]pyrimidine-2,4(1H,3H)-dione (racemate)

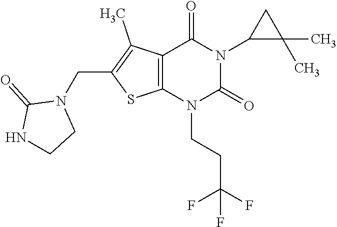 [SMILES: Cc1c(CN2CCNC2=O)sc2c1c(=O)n(C1CC1(C)C)c(=O)n2CCC(F)(F)F]

Analogously to Ex. 90, 699 mg (1.40 mmol, 84% purity) of the compound from Ex. 129A in 50 ml of dioxane were reacted with 352 mg (2.105 mmol) of CDI. 348 mg (64% of theory) of the title compound were obtained.

¹H-NMR (400 MHz, DMSO-d₆, δ/ppm): 6.55 (s, 1H), 4.35 (s, 2H), 4.07 (td, 2H), 3.29-3.17 (m, 4H), 2.81-2.69 (m, 2H), 2.42-2.36 (m, 4H), 1.17 (s, 3H), 1.04 (dd, 1H), 0.85 (s, 3H), 0.72 (dd, 1H).

LC/MS (Method 4, ESIpos): Rₜ=1.14 min, m/z=445 [M+H]⁺.

### Example 101

### 3-(2,2-Dimethylcyclopropyl)-5-methyl-6-[(2-oxoimidazolidin-1-yl)methyl]-1-(3,3,3-trifluoropropyl)thieno[2,3-d]pyrimidine-2,4(1H,3H)-dione (enantiomer 1)

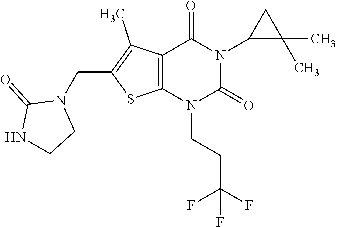 [SMILES: Cc1c(CN2CCNC2=O)sc2c1c(=O)n(C1CC1(C)C)c(=O)n2CCC(F)(F)F]

348 mg of the racemic compound from Ex. 100 were separated into the enantiomers by means of preparative HPLC on a chiral phase [column: Daicel Chiralpak IC, 5 μm, 250 mm×30 mm, eluent A: acetonitrile+0.1% diethylamine (99%), eluent B: MTBE+0.1% diethylamine (99%), isocratic 50% A+50% B; flow rate: 50 ml/min; detection: 254 nm]. The product fractions were concentrated on a rotary evaporator, admixed with tert-butanol and freeze-dried. In this way, 149 mg (85% of theory) of the title compound (enantiomer 1) and 151 mg (86% of theory) of enantiomer 2 (see Ex. 102) were obtained.

¹H-NMR (400 MHz, DMSO-d₆, δ/ppm): 6.55 (br. s, 1H), 4.35 (s, 2H), 4.07 (br. s, 2H), 3.27-3.18 (m, 4H), 2.82-2.69 (m, 2H), 2.41-2.36 (m, 4H), 1.16 (s, 3H), 1.04 (br. t, 1H), 0.85 (s, 3H), 0.72 (t, 1H).

Chiral analytical HPLC [column: Daicel Chiralpak IC, 3 μm, 100 mm×4.6 mm, eluent A: acetonitrile+0.1% diethylamine (99%), eluent B: MTBE+0.1% diethylamine (99%), isocratic: 50% A+50% B; flow rate: 1.4 ml/min; temperature: 25° C.; detection: 254 nm]: Rₜ=3.63 min.

### Example 102

### 3-(2,2-Dimethylcyclopropyl)-5-methyl-6-[(2-oxoimidazolidin-1-yl)methyl]-1-(3,3,3-trifluoropropyl)thieno[2,3-d]pyrimidine-2,4(1H,3H)-dione (enantiomer 2)

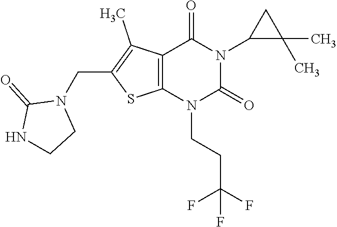 [SMILES: Cc1c(CN2CCNC2=O)sc2c1c(=O)n(C1CC1(C)C)c(=O)n2CCC(F)(F)F]

The title compound (151 mg) was obtained as the second enantiomer in the preparative HPLC separation of the racemate from Ex. 100 (see Ex. 101).

¹H-NMR (400 MHz, DMSO-d₆, δ/ppm): 6.55 (br. s, 1H), 4.35 (s, 2H), 4.07 (td, 2H), 3.29-3.17 (m, 4H), 2.81-2.68 (m, 2H), 2.42-2.36 (m, 4H), 1.16 (s, 3H), 1.04 (dd, 1H), 0.85 (s, 3H), 0.72 (dd, 1H).

Chiral analytical HPLC [column: Daicel Chiralpak IC, 3 μm, 100 mm×4.6 mm, eluent A: acetonitrile+0.1% diethylamine (99%), eluent B: MTBE+0.1% diethylamine (99%), isocratic: 50% A+50% B; flow rate: 1.4 ml/min; temperature: 25° C.; detection: 254 nm]: Rₜ=4.42 min.

### Example 103

### 3-(2,2-Dimethylcyclopropyl)-1-(2-methoxyethyl)-5-methyl-6-[(2-oxoimidazolidin-1-yl)methyl]thieno[2,3-d]pyrimidine-2,4(1H,3H)-dione (racemate)

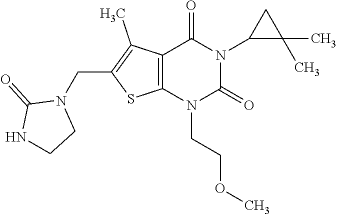 [SMILES: COCCn1c(=O)n(C2CC2(C)C)c(=O)c2c(C)c(CN3CCNC3=O)sc21]

Analogously to Ex. 90, 465 mg (1.1 mmol, 90% purity) of the compound from Ex. 130A in 45 ml of dioxane were reacted with 276 ml (1.65 mmol) of CDI. 310 mg (68% of theory) of the title compound were obtained.

¹H-NMR (400 MHz, DMSO-d₆, δ/ppm): 6.53 (s, 1H), 4.33 (s, 2H), 4.10-3.99 (m, 1H), 3.98-3.87 (m, 1H), 3.66-3.55 (m, 2H), 3.29-3.16 (m, 6H), 2.42-2.34 (m, 4H), 1.16 (s, 3H), 1.03 (dd, 1H), 0.85 (s, 3H), 0.72 (dd, 1H).

LC/MS (Method 4, ESIpos): Rₜ=0.99 min, m/z=407 [M+H]⁺.

### Example 104

### 3-(2,2-Dimethylcyclopropyl)-1-(2-methoxyethyl)-5-methyl-6-[(2-oxoimidazolidin-1-yl)methyl]thieno[2,3-d]pyrimidine-2,4(1H,3H)-dione (enantiomer 1)

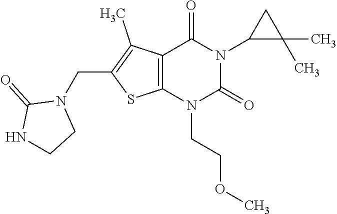 [SMILES: COCCn1c(=O)n(C2CC2(C)C)c(=O)c2c(C)c(CN3CCNC3=O)sc21]

264 mg of the racemic compound from Ex. 103 were separated into the enantiomers by means of preparative SFC-HPLC on a chiral phase [column: Daicel Chiralpak IC, 5 μm, 250 mm×30 mm, eluent A: carbon dioxide, eluent B: ethanol, isocratic 51% A+49% B; flow rate: 100 ml/min; temperature: 40° C.; BPR: 150 bar; MWD: 254 nm]. The product fractions were concentrated on a rotary evaporator, admixed with tert-butanol and freeze-dried. 114 mg (86% of theory) of the title compound (enantiomer 1) and 116 mg (87% of theory) of enantiomer 2 (see Ex. 105) were obtained.

¹H-NMR (400 MHz, DMSO-d₆, δ/ppm): 6.53 (s, 1H), 4.33 (s, 2H), 4.10-3.99 (m, 1H), 3.97-3.87 (m, 1H), 3.65-3.56 (m, 2H), 3.28-3.16 (m, 6H), 2.41-2.34 (m, 4H), 1.16 (s, 3H), 1.03 (dd, 1H), 0.85 (s, 3H), 0.72 (dd, 1H).

Chiral analytical SFC-HPLC [column: Daicel Chiralpak IC, 5 μm, 100 mm×4.6 mm, eluent A: carbon dioxide, eluent B: ethanol, isocratic 51% A+49% B; flow rate: 4.0 ml/min; temperature: 37.5° C.; BPR: 100 bar; MWD: 254 nm]: Rₜ=2.74 min.

### Example 105

### 3-(2,2-Dimethylcyclopropyl)-1-(2-methoxyethyl)-5-methyl-6-[(2-oxoimidazolidin-1-yl)methyl]thieno[2,3-d]pyrimidine-2,4(1H,3H)-dione (enantiomer 2)

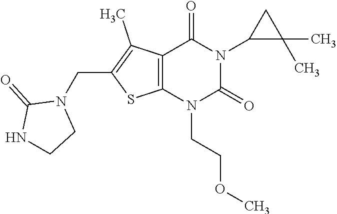 [SMILES: COCCn1c(=O)n(C2CC2(C)C)c(=O)c2c(C)c(CN3CCNC3=O)sc21]

The title compound (116 mg) was obtained as the second enantiomer in the preparative HPLC separation of the racemate from Ex. 103 (see Ex. 104).

¹H-NMR (400 MHz, DMSO-d₆, δ/ppm): 6.53 (s, 1H), 4.33 (s, 2H), 4.10-3.99 (m, 1H), 3.98-3.87 (m, 1H), 3.66-3.55 (m, 2H), 3.29-3.17 (m, 6H), 2.42-2.34 (m, 4H), 1.16 (s, 3H), 1.03 (dd, 1H), 0.85 (s, 3H), 0.72 (dd, 1H).

Chiral analytical SFC-HPLC [column: Daicel Chiralpak IC, 5 μm, 100 mm×4.6 mm, eluent A: carbon dioxide, eluent B: ethanol, isocratic 51% A+49% B; flow rate: 4.0 ml/min; temperature: 37.5° C.; BPR: 100 bar; MWD: 254 nm]: Rₜ=3.42 min.

### Example 106

### 3-(1-Ethylcyclopropyl)-5-methyl-6-[(2-oxoimidazolidin-1-yl)methyl]-1-(3,3,3-trifluoropropyl)thieno[2,3-d]pyrimidine-2,4(1H,3H)-dione

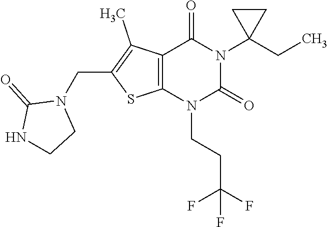 [SMILES: CCC1(n2c(=O)c3c(C)c(CN4CCNC4=O)sc3n(CCC(F)(F)F)c2=O)CC1]

Analogously to the method described in Ex. 3, 159 mg (0.342 mmol, 90% purity) of the compound from Ex. 131A and 67 mg (0.410 mmol) of CDI were used to prepare 82 mg (53% of theory) of the title compound.

¹H-NMR (400 MHz, DMSO-d₆, δ/ppm): 6.52 (s, 1H), 4.35 (s, 2H), 4.14-3.96 (m, 2H), 3.28-3.17 (m, 4H), 2.82-2.65 (m, 2H), 2.38 (s, 3H), 1.69 (q, 2H), 1.01-0.87 (m, 2H), 0.86-0.77 (m, 2H), 0.81 (t, 3H).

LC/MS (Method 1, ESIpos): Rₜ=1.69 min, m/z=445.15 [M+H]⁺.

### Example 107

### 3-(1-Ethylcyclopropyl)-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

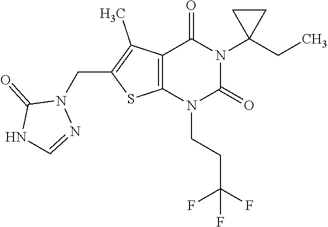 [SMILES: CCC1(n2c(=O)c3c(C)c(Cn4nc[nH]c4=O)sc3n(CCC(F)(F)F)c2=O)CC1]

Analogously to the method described in Ex. 2, 116 mg (0.217 mmol) of the compound from Ex. 270A were used to prepare 66 mg (68% of theory) of the title compound.

¹H-NMR (400 MHz, DMSO-d₆, δ/ppm): 11.59 (br. s, 1H), 7.83 (s, 1H), 4.94 (s, 2H), 4.12-3.96 (m, 2H), 2.80-2.65 (m, 2H), 2.45 (s, 3H), 1.69 (q, 2H), 1.00-0.86 (m, 2H), 0.86-0.74 (m, 2H), 0.81 (t, 3H).

LC/MS (Method 1, ESIpos): Rₜ=1.61 min, m/z=444.13 [M+H]⁺.

### Example 108

### 3-(1-Ethylcyclopropyl)-1-(2-methoxyethyl)-5-methyl-6-[(2-oxoimidazolidin-1-yl)methyl]thieno[2,3-d]pyrimidine-2,4(1H,3H)-dione

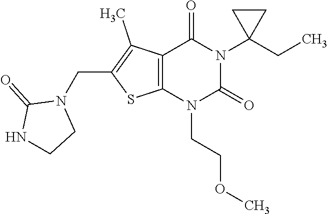 [SMILES: CCC1(n2c(=O)c3c(C)c(CN4CCNC4=O)sc3n(CCOC)c2=O)CC1]

Analogously to the method described in Ex. 3, 247 mg (0.584 mmol, 90% purity) of the compound from Ex. 132A and 114 mg (0.701 mmol) of CDI were used to prepare 104 mg (43% of theory) of the title compound.

¹H-NMR (400 MHz, DMSO-d₆, δ/ppm): 6.51 (s, 1H), 4.33 (s, 2H), 4.07-3.87 (m, 2H), 3.60 (t, 2H), 3.28-3.15 (m, 4H), 3.23 (s, 3H), 2.37 (s, 3H), 1.76-1.62 (m, 2H), 0.99-0.87 (m, 2H), 0.86-0.75 (m, 2H), 0.81 (t, 3H).

LC/MS (Method 1, ESIpos): Rₜ=1.44 min, m/z=407.17 [M+H]⁺.

### Example 109

### 3-(1-Ethylcyclopropyl)-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione

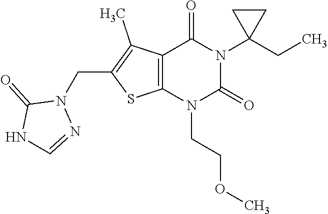 [SMILES: CCC1(n2c(=O)c3c(C)c(Cn4nc[nH]c4=O)sc3n(CCOC)c2=O)CC1]

Analogously to the method described in Ex. 50, 169 mg (0.324 mmol, 95% purity) of the compound from Ex. 271A were used to prepare 62 mg (47% of theory) of the title compound.

¹H-NMR (400 MHz, DMSO-d₆, δ/ppm): 11.58 (br. s, 1H), 7.83 (s, 1H), 4.91 (s, 2H), 4.06-3.86 (m, 2H), 3.59 (t, 2H), 3.22 (s, 3H), 2.43 (s, 3H), 1.69 (q, 2H), 0.99-0.86 (m, 2H), 0.86-0.74 (m, 2H), 0.81 (t, 3H).

LC/MS (Method 1, ESIpos): Rₜ=1.33 min, m/z=406.15 [M+H]⁺.

### Example 110

### 3-Cyclobutyl-5-methyl-6-[(2-oxoimidazolidin-1-yl)methyl]-1-(3,3,3-trifluoropropyl)thieno[2,3-d]pyrimidine-2,4(1H,3H)-dione

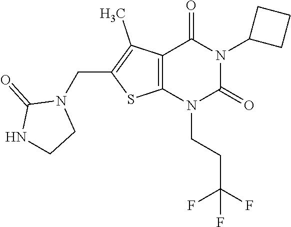 [SMILES: Cc1c(CN2CCNC2=O)sc2c1c(=O)n(C1CCC1)c(=O)n2CCC(F)(F)F]

Analogously to the method described in Ex. 3, 500 mg (0.989 mmol, 80% purity) of the compound from Ex. 133A and 192 mg (1.19 mmol) of CDI were used to prepare 138 mg (32% of theory) of the title compound.

¹H-NMR (400 MHz, DMSO-d₆, δ/ppm): 6.53 (s, 1H), 5.21 (quin, 1H), 4.36 (s, 2H), 4.06 (t, 2H), 3.29-3.17 (m, 4H), 2.92-2.66 (m, 4H), 2.39 (s, 3H), 2.16 (qt, 2H), 1.89-1.63 (m, 2H).

LC/MS (Method 1, ESIpos): Rₜ=1.77 min, m/z=431.14 [M+H]⁺.

### Example 111

### [1-{[3-Cyclobutyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

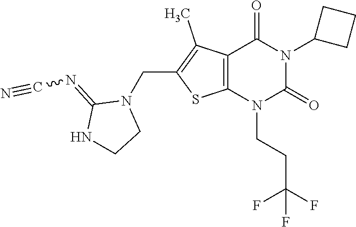 [SMILES: Cc1c(CN2CCNC2=NC#N)sc2c1c(=O)n(C1CCC1)c(=O)n2CCC(F)(F)F]

500 mg (0.989 mmol, 80% purity) of the compound from Ex. 133A were dissolved in 10 ml of DMF, and 217 mg (1.48 mmol) of dimethyl N-cyanodithioiminocarbonate and 273 mg (1.98 mmol) of potassium carbonate were added. The mixture was stirred at 80° C. in a microwave oven (Biotage Initiator with dynamic control of irradiation power) for 4 h. Thereafter, the reaction mixture was concentrated to dryness and then purified by means of preparative HPLC (Method 11). The product fractions were combined, concentrated by evaporation and dried under high vacuum. 224 mg (49% of theory) of the title compound were obtained.

¹H-NMR (400 MHz, DMSO-d₆, δ/ppm): 8.08 (s, 1H), 5.21 (quin, 1H), 4.49 (s, 2H), 4.08 (t, 2H), 3.52-3.36 (m, 4H), 2.91-2.68 (m, 4H), 2.40 (s, 3H), 2.16 (qt, 2H), 1.88-1.65 (m, 2H).

LC/MS (Method 2, ESIpos): Rₜ=0.98 min, m/z=455 [M+H]⁺.

### Example 112

### Methyl [1-{[3-cyclobutyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

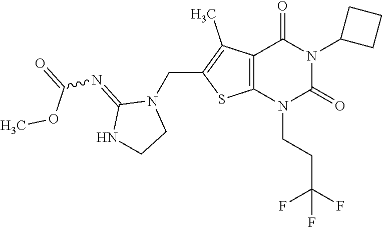 [SMILES: COC(=O)N=C1NCCN1Cc1sc2c(c1C)c(=O)n(C1CCC1)c(=O)n2CCC(F)(F)F]

Analogously to the method described in Ex. 26, 500 mg (0.989 mmol, 80% purity) of the compound from Ex. 133A and 308 mg (1.98 mmol) of methyl (dichloromethylene)carbamate were used to prepare 204 mg (42% of theory) of the title compound.

¹H-NMR (400 MHz, DMSO-d₆, δ/ppm): 8.02 (s, 1H), 5.21 (quin, 1H), 4.57 (s, 2H), 4.05 (t, 2H), 3.53 (s, 3H), 3.50-3.41 (m, 2H), 3.38-3.29 (m, 2H, partially concealed by water signal), 2.91-2.67 (m, 4H), 2.41 (s, 3H), 2.16 (qt, 2H), 1.88-1.63 (m, 2H).

LC/MS (Method 1, ESIpos): Rₜ=1.79 min, m/z=488.16 [M+H]⁺.

### Example 113

### 3-Cyclobutyl-6-[(2,3-dioxopiperazin-1-yl)methyl]-5-methyl-1-(3,3,3-trifluoropropyl)thieno[2,3-d]pyrimidine-2,4(1H,3H)-dione

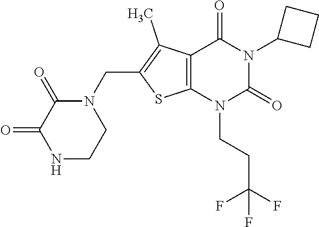 [SMILES: Cc1c(CN2CCNC(=O)C2=O)sc2c1c(=O)n(C1CCC1)c(=O)n2CCC(F)(F)F]

To a solution of 500 mg (0.989 mmol, 80% purity) of the compound from Ex. 133A in 40 ml of ethanol were added 252 μl (1.83 mmol) of diethyl oxalate, and the mixture was stirred at 80° C. for about 16 h. Thereafter, the reaction mixture was concentrated and then purified by means of preparative HPLC (Method 11). After concentration of the product fraction and drying under high vacuum, 118 mg (26% of theory) of the title compound were obtained.

¹H-NMR (400 MHz, DMSO-d₆, δ/ppm): 8.62 (br. s, 1H), 5.20 (quin, 1H), 4.70 (s, 2H), 4.07 (t, 2H), 3.53-3.43 (m, 2H), 3.34-3.28 (m, 2H, almost entirely covered by water signal), 2.91-2.68 (m, 4H), 2.42 (s, 3H), 2.23-2.10 (m, 2H), 1.88-1.63 (m, 2H).

LC/MS (Method 2, ESIneg): Rₜ=0.85 min, m/z=503 [M−H+HCOOH]⁻.

### Example 114

### 3-Cyclobutyl-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

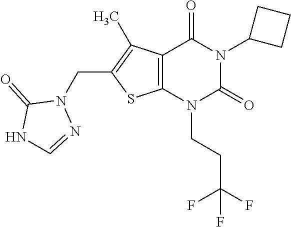 [SMILES: Cc1c(Cn2nc[nH]c2=O)sc2c1c(=O)n(C1CCC1)c(=O)n2CCC(F)(F)F]

121 mg (0.233 mmol) of the compound from Ex. 272A were dissolved in a mixture of 5.6 ml each of methanol and trimethyl orthoformate, and 582 μl (2.33 mmol) of a 4 M solution of hydrogen chloride in dioxane were added at RT. After 1 h and after 2 h, a further 291 μl (1.17 mmol) each time of the 4 M solution of hydrogen chloride in dioxane were added. After a total of 16 h, the reaction mixture was diluted with water and extracted with ethyl acetate. The organic extract was concentrated and the residue was purified by means of preparative HPLC (Method 11). After concentration of the product fractions and drying under high vacuum, 41 mg (41% of theory) of the title compound were obtained.

¹H-NMR (400 MHz, DMSO-d₆, δ/ppm): 11.60 (br. s, 1H), 7.84 (s, 1H), 5.20 (quin, 1H), 4.94 (s, 2H), 4.05 (t, 2H), 2.90-2.64 (m, 4H), 2.45 (s, 3H), 2.23-2.10 (m, 2H), 1.88-1.63 (m, 2H).

LC/MS (Method 1, ESIpos): Rₜ=1.64 min, m/z=430.12 [M+H]⁺.

### Example 115

### 3-Cyclobutyl-1-(2-methoxyethyl)-5-methyl-6-[(2-oxoimidazolidin-1-yl)methyl]thieno[2,3-d]pyrimidine-2,4(1H,3H)-dione

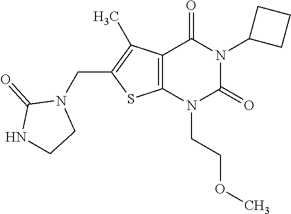 [SMILES: COCCn1c(=O)n(C2CCC2)c(=O)c2c(C)c(CN3CCNC3=O)sc21]

To a solution of 650 mg (1.42 mmol, 80% purity) of the compound from Ex. 134A and 297 μl (2.13 mmol) of triethylamine in 15 ml of THF were added 276 mg (1.70 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was stirred in a little acetonitrile at RT, then filtered off with suction and dried under high vacuum. 170 mg (30% of theory) of the title compound were obtained.

¹H-NMR (400 MHz, DMSO-d₆, δ/ppm): 6.52 (s, 1H), 5.22 (quin, 1H), 4.33 (s, 2H), 3.99 (t, 2H), 3.62 (t, 2H), 3.28-3.16 (m, 4H), 3.24 (s, 3H), 2.92-2.77 (m, 2H), 2.37 (s, 3H), 2.22-2.09 (m, 2H), 1.87-1.64 (m, 2H).

LC/MS (Method 2, ESIpos): Rₜ=0.81 min, m/z=393 [M+H]⁺.

### Example 116

### [1-{[3-Cyclobutyl-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

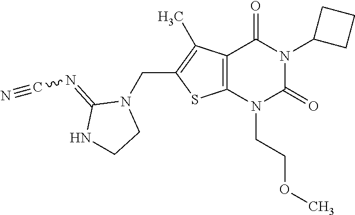 [SMILES: COCCn1c(=O)n(C2CCC2)c(=O)c2c(C)c(CN3CCNC3=NC#N)sc21]

600 mg (1.31 mmol, 80% purity) of the compound from Ex. 134A were dissolved in 12 ml of DMF, and 287 mg (1.97 mmol) of dimethyl N-cyanodithioiminocarbonate and 362 mg (2.62 mmol) of potassium carbonate were added. The mixture was stirred at 80° C. in a microwave oven (Biotage Initiator with dynamic control of irradiation power) for 4 h. Thereafter, the reaction mixture was concentrated to dryness and then purified by means of preparative HPLC (Method 11). The product fractions were combined, concentrated by evaporation and dried under high vacuum. 335 mg (61% of theory) of the title compound were obtained.

¹H-NMR (400 MHz, DMSO-d₆, δ/ppm): 8.07 (s, 1H), 5.21 (quin, 1H), 4.46 (s, 2H), 4.00 (t, 2H), 3.63 (t, 2H), 3.50-3.37 (m, 4H), 3.24 (s, 3H), 2.91-2.77 (m, 2H), 2.39 (s, 3H), 2.16 (qt, 2H), 1.88-1.64 (m, 2H).

LC/MS (Method 2, ESIpos): Rₜ=0.87 min, m/z=417 [M+H]⁺.

### Example 117

### Methyl [1-{[3-cyclobutyl-1-(2-methoxyethyl)-5-methyl-2,4-dioxo-1,2,3,4-tetrahydrothieno[2,3-d]pyrimidin-6-yl]methyl}imidazolidin-2-ylidene]carbamate

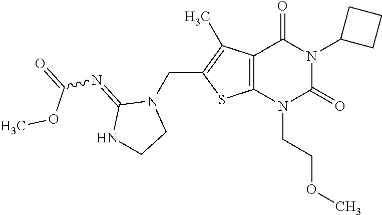 [SMILES: COCCn1c(=O)n(C2CCC2)c(=O)c2c(C)c(CN3CCNC3=NC(=O)OC)sc21]

Analogously to the method described in Ex. 26, 650 mg (1.42 mmol, 80% purity) of the compound from Ex. 134A and 442 mg (2.84 mmol) of methyl (dichloromethylene)carbamate were used to prepare 336 mg (52% of theory) of the title compound. A difference from the purification process described above was that the preparative HPLC here followed the MPLC.

¹H-NMR (400 MHz, DMSO-d₆, δ/ppm): 8.06 (br. s, 1H), 5.22 (quin, 1H), 4.56 (s, 2H), 3.98 (t, 2H), 3.62 (t, 2H), 3.54 (s, 3H), 3.50-3.42 (m, 2H), 3.36-3.29 (m, 2H, partially concealed by water signal), 3.23 (s, 3H), 2.91-2.77 (m, 2H), 2.40 (s, 3H), 2.16 (qt, 2H), 1.88-1.64 (m, 2H).

LC/MS (Method 2, ESIpos): Rₜ=0.82 min, m/z=450 [M+H]⁺.

### Example 118

### 3-Cyclobutyl-6-[(2,3-dioxopiperazin-1-yl)methyl]-1-(2-methoxyethyl)-5-methylthieno[2,3-d]pyrimidine-2,4(1H,3H)-dione

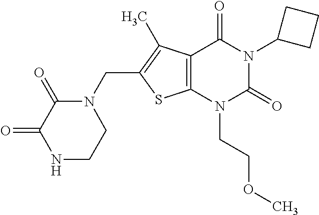 [SMILES: COCCn1c(=O)n(C2CCC2)c(=O)c2c(C)c(CN3CCNC(=O)C3=O)sc21]

To a solution of 480 mg (1.05 mmol, 80% purity) of the compound from Ex. 134A in 35 ml of ethanol were added 267 μl (1.94 mmol) of diethyl oxalate, and the mixture was stirred at 80° C. for about 16 h. Thereafter, the reaction mixture was concentrated and then prepurified by means of preparative HPLC (Method 11). After the product fraction had been concentrated, the solids obtained were stirred in a little acetonitrile at RT. After filtration with suction and drying under high vacuum, 74 mg (16% of theory) of the title compound were obtained.

¹H-NMR (400 MHz, DMSO-d₆, δ/ppm): 8.61 (br. s, 1H), 5.21 (quin, 1H), 4.68 (s, 2H), 3.99 (t, 2H), 3.62 (t, 2H), 3.52-3.43 (m, 2H), 3.34-3.28 (m, 2H, almost entirely covered by water signal), 3.24 (s, 3H), 2.92-2.76 (m, 2H), 2.41 (s, 3H), 2.22-2.09 (m, 2H), 1.89-1.63 (m, 2H).

LC/MS (Method 2, ESIneg): Rₜ=0.73 min, m/z=465 [M−H+HCOOH]⁻.

### Example 119

### 3-Cyclobutyl-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione

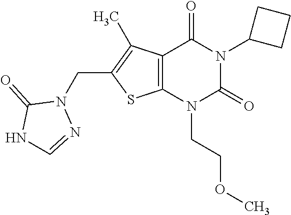 [SMILES: COCCn1c(=O)n(C2CCC2)c(=O)c2c(C)c(Cn3nc[nH]c3=O)sc21]

110 mg (0.228 mmol) of the compound from Ex. 273A were dissolved in a mixture of 5.5 ml each of methanol and trimethyl orthoformate, and 571 μl (2.28 mmol) of a 4 M solution of hydrogen chloride in dioxane were added at RT. After 3 h and after 15 h, a further 285 μl (1.14 mmol) each time of the 4 M solution of hydrogen chloride in dioxane were added. After a total of 40 h, the reaction mixture was diluted with water and extracted with ethyl acetate. The organic extract was concentrated, and the residue obtained was stirred in a mixture of acetonitrile, methanol and water. The solids were filtered off with suction and dried under high vacuum. The filtrate was purified by means of preparative HPLC (Method 11). After concentration of the product fractions, drying under high vacuum and combination with the solids isolated beforehand, 70 mg (78% of theory) of the title compound were obtained.

¹H-NMR (400 MHz, DMSO-d₆, δ/ppm): 11.59 (br. s, 1H), 7.83 (s, 1H), 5.21 (quin, 1H), 4.92 (s, 2H), 3.97 (t, 2H), 3.61 (t, 2H), 3.23 (s, 3H), 2.90-2.76 (m, 2H), 2.43 (s, 3H), 2.21-2.10 (m, 2H), 1.87-1.63 (m, 2H).

LC/MS (Method 1, ESIpos): Rₜ=1.39 min, m/z=392.14 [M+H]⁺.

### Example 120

### 3-(3,3-Difluorocyclobutyl)-5-methyl-6-[(2-oxoimidazolidin-1-yl)methyl]-1-(3,3,3-trifluoropropyl)thieno[2,3-d]pyrimidine-2,4(1H,3H)-dione

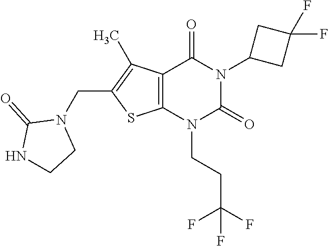 [SMILES: Cc1c(CN2CCNC2=O)sc2c1c(=O)n(C1CC(F)(F)C1)c(=O)n2CCC(F)(F)F]

200 mg (0.39 mmol, 87% purity) of the compound from Ex. 135A were dissolved in 3 ml of THF, and 77 mg (0.47 mmol) of CDI were added. The mixture was stirred at RT for 16 h. The reaction solution was then concentrated on a rotary evaporator. The residue was taken up in ethyl acetate and washed successively with 1 M hydrochloric acid, saturated aqueous sodium hydrogencarbonate solution and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 29 mg (16% of theory) of the title compound.

¹H-NMR (400 MHz, DMSO-d₆, δ/ppm): 6.54 (s, 1H), 5.15 (td, 1H), 4.37 (s, 2H), 4.07 (t, 2H), 3.54-3.39 (m, 2H), 3.28-3.18 (m, 4H), 2.91-2.67 (m, 4H), 2.39 (s, 3H).

LC/MS (Method 2, ESIpos): Rₜ=0.95 min, m/z=467 [M+H]⁺.

### Example 121

### [1-{[3-(3,3-Difluorocyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

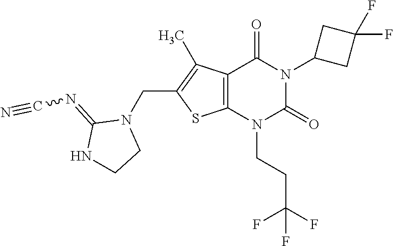 [SMILES: Cc1c(CN2CCNC2=NC#N)sc2c1c(=O)n(C1CC(F)(F)C1)c(=O)n2CCC(F)(F)F]

200 mg (0.40 mmol, 87% purity) of the compound from Ex. 135A were dissolved in 16 ml of DMF, and 109 mg (0.79 mmol) of potassium carbonate and 87 mg (0.593 mmol) of dimethyl N-cyanodithioiminocarbonate were added. The mixture was stirred at 80° C. in a microwave oven (Biotage Initiator with dynamic control of irradiation power) for 3 h. Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue was purified by preparative HPLC (Method 13). Concentration of the product fractions and drying under high vacuum gave 15 mg (8% of theory) of the title compound.

¹H-NMR (400 MHz, DMSO-d₆, δ/ppm): 8.09 (s, 1H), 5.17-5.12 (m, 1H), 4.50 (s, 2H), 4.09 (t, 2H), 3.53-3.39 (m, 6H), 2.90-2.71 (m, 4H), 2.41 (s, 3H).

LC/MS (Method 2, ESIpos): Rₜ=1.32 min, m/z=491 [M+H]⁺.

### Example 122

### Methyl [1-{[3-(3,3-difluorocyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

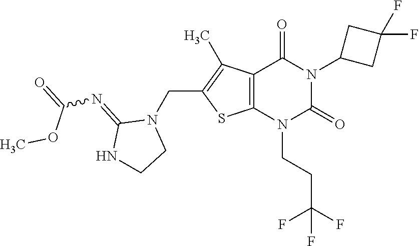 [SMILES: COC(=O)N=C1NCCN1Cc1sc2c(c1C)c(=O)n(C1CC(F)(F)C1)c(=O)n2CCC(F)(F)F]

250 mg (0.50 mmol, 87% purity) of the compound from Ex. 135A and 138 μl (0.99 mmol) of triethylamine were dissolved in 23 ml of dichloromethane, and 77 mg (0.49 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 177 mg (68% of theory) of the title compound.

¹H-NMR (400 MHz, DMSO-d₆, δ/ppm): 5.27-5.19 (m, 1H), 4.80 (s, 2H), 4.17 (dd, 2H), 3.91 (s, 3H), 3.79 (dd, 2H), 3.65 (dd, 2H), 3.57-3.47 (m, 2H), 2.87-2.79 (m, 2H), 2.77-2.67 (m, 2H), 2.48 (s, 3H).

LC/MS (Method 2, ESIpos): Rₜ=0.93 min, m/z=524 [M+H]⁺.

### Example 123

### 3-(3,3-Difluorocyclobutyl)-6-[(2,3-dioxopiperazin-1-yl)methyl]-5-methyl-1-(3,3,3-trifluoropropyl)thieno[2,3-d]pyrimidine-2,4(1H,3H)-dione

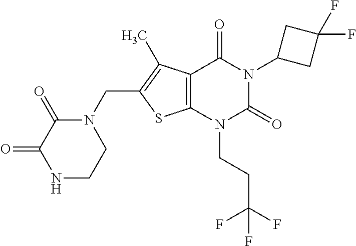 [SMILES: Cc1c(CN2CCNC(=O)C2=O)sc2c1c(=O)n(C1CC(F)(F)C1)c(=O)n2CCC(F)(F)F]

200 mg (0.40 mmol, 87% purity) of the compound from Example 135A were dissolved in 16 ml of ethanol, and 108 mg (0.73 mmol) of diethyl oxalate were added. The mixture was stirred at 80° C. for 3 days. Thereafter, the reaction solution was concentrated on a rotary evaporator. The residue obtained was purified by preparative HPLC (Method 13). Concentration of the product fractions and drying under high vacuum gave 96 mg (49% of theory) of the title compound.

¹H-NMR (400 MHz, DMSO-d₆, δ/ppm): 8.63 (br. s, 1H), 5.19-5.08 (m, 1H), 4.71 (s, 2H), 4.08 (t, 2H), 3.52-3.38 (m, 4H), 2.92-2.71 (m, 4H), 2.43 (s, 3H).

LC/MS (Method 2, ESIpos): Rₜ=0.88 min, m/z=495 [M+H]⁺.

### Example 124

### 3-(3,3-Difluorocyclobutyl)-5-methyl-6-[(5-oxo-4,5-dihydro-H-1,2,4-triazol-1-yl)methyl]-1-(3,3,3-trifluoropropyl)thieno[2,3-d]pyrimidine-2,4(1H,3H)-dione

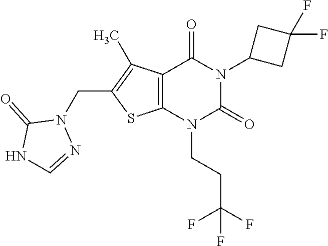 [SMILES: Cc1c(Cn2nc[nH]c2=O)sc2c1c(=O)n(C1CC(F)(F)C1)c(=O)n2CCC(F)(F)F]

756 mg (1.07 mmol, 79% purity) of the compound from Ex. 274A were initially charged in 26.3 ml of trimethyl orthoformate and 26.3 ml of methanol, and 2.7 ml (10.7 mmol) of a 4 M solution of hydrogen chloride in dioxane were added. Since the conversion was still incomplete after stirring at RT for 18 h, a further 1.3 ml of 4 M hydrogen chloride in dioxane was added and the mixture was stirred for a further 4 h. Thereafter, another 1.3 ml of 4 M hydrogen chloride in dioxane was added, and the mixture was stirred for a further 2.5 h. Then water was added to the reaction mixture, which was extracted with ethyl acetate. The organic phase was concentrated and the residue was purified by means of preparative HPLC (Method 13). The product fractions were combined and concentrated, and the residue was dried under high vacuum. 267 mg (54% of theory) of the title compound were obtained.

¹H-NMR (400 MHz, DMSO-d₆, δ/ppm): 11.60 (br. s, 1H), 7.84 (s, 1H), 5.18-5.09 (m, 1H), 4.95 (s, 2H), 4.06 (t, 2H), 3.52-3.41 (m, 2H), 2.90-2.67 (m, 4H), 2.45 (s, 3H).

LC/MS (Method 2, ESIpos): Rₜ=0.88 min, m/z=466 [M+H]⁺.

### Example 125

### 3-(3,3-Difluorocyclobutyl)-1-(2-methoxyethyl)-5-methyl-6-[(2-oxoimidazolidin-1-yl)methyl]thieno[2,3-d]pyrimidine-2,4(1H,3H)-dione

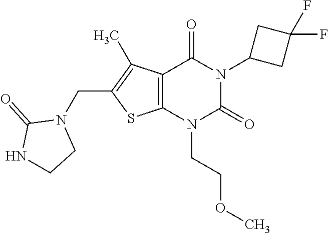 [SMILES: COCCn1c(=O)n(C2CC(F)(F)C2)c(=O)c2c(C)c(CN3CCNC3=O)sc21]

300 mg (0.51 mmol, 69% purity) of the compound from Ex. 136A were dissolved in 4 ml of THF, and 99 mg (0.62 mmol) of CDI and 107 μl (0.77 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated sodium hydrogencarbonate solution and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 73 mg (33% of theory) of the title compound.

¹H-NMR (400 MHz, DMSO-d₆, δ/ppm): 6.53 (br. s, 1H), 5.20-5.11 (m, 1H), 4.35 (s, 2H), 4.00 (t, 2H), 3.62 (t, 2H), 3.54-3.40 (m, 2H), 3.25-3.18 (m, 7H), 2.89-2.79 (m, 2H), 2.38 (s, 3H).

LC/MS (Method 2, ESIpos): Rₜ=0.85 min, m/z=429 [M+H]⁺.

### Example 126

### [1-{[3-(3,3-Difluorocyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

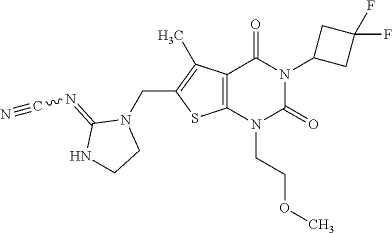 [SMILES: COCCn1c(=O)n(C2CC(F)(F)C2)c(=O)c2c(C)c(CN3CCNC3=NC#N)sc21]

377 mg (0.64 mmol, 69% purity) of the compound from Example 136A were dissolved in 25 ml of DMF, and 178 mg (1.29 mmol) of potassium carbonate were added. The mixture was stirred at RT for 15 min, then 141 mg (0.97 mmol) of dimethyl N-cyanodithioiminocarbonate were added. The reaction mixture was stirred at 80° C. in a microwave oven (Biotage Initiator with dynamic control of irradiation power) for 3 h. Thereafter, the reaction mixture was taken up in ethyl acetate and washed successively with saturated sodium hydrogencarbonate solution, water and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 52 mg (17% of theory) of the title compound.

¹H-NMR (400 MHz, DMSO-d₆, δ/ppm): 8.09 (s, 1H), 5.20-5.12 (m, 1H), 4.48 (s, 2H), 4.01 (t, 2H), 3.63 (t, 2H), 3.50-3.30 (m, 6H), 3.26 (s, 3H), 2.88-2.80 (m, 2H), 2.39 (s, 3H).

LC/MS (Method 2, ESIpos): Rₜ=0.89 min, m/z=453 [M+H]⁺.

### Example 127

### Methyl [1-{[3-(3,3-difluorocyclobuty)-1-(2-methoxyethyl)-5-methyl-2,4-dioxo-1,2,3,4-tetrahydrothieno[2,3-d]pyrimidin-6-yl]methyl}imidazolidin-2-ylidene]carbamate

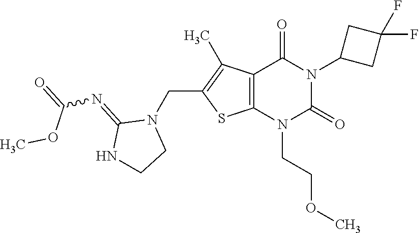 [SMILES: COCCn1c(=O)n(C2CC(F)(F)C2)c(=O)c2c(C)c(CN3CCNC3=NC(=O)OC)sc21]

250 mg (0.54 mmol, 69% purity) of the compound from Ex. 136A and 151 μl (1.08 mmol) of triethylamine were dissolved in 25 ml of dichloromethane, and 84 mg (0.54 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 104 mg (40% of theory) of the title compound.

¹H-NMR (400 MHz, DMSO-d₆, δ/ppm): 5.20-5.12 (m, 1H), 4.72 (br. s, 2H), 4.01 (t, 2H), 3.72 (br. s, 3H), 3.63 (t, 2H), 3.58 (d, 2H), 3.57-3.46 (m, 3H), 2.90-2.80 (m, 2H), 2.41 (s, 3H).

LC/MS (Method 1, ESIpos): Rₜ=0.84 min, m/z=486 [M+H]⁺.

### Example 128

### 3-(3,3-Difluorocyclobutyl)-6-[(2,3-dioxopiperazin-1-yl)methyl]-1-(2-methoxyethyl)-5-methylthieno[2,3-d]pyrimidine-2,4(1H,3H)-dione

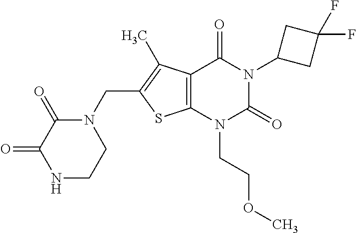 [SMILES: COCCn1c(=O)n(C2CC(F)(F)C2)c(=O)c2c(C)c(CN3CCNC(=O)C3=O)sc21]

250 mg (0.43 mmol, 69% purity) of the compound from Example 136A were dissolved in 17 ml of ethanol, and 116 mg (0.79 mmol) of diethyl oxalate were added. The mixture was stirred at 80° C. for 2 days. Thereafter, the reaction solution was concentrated on a rotary evaporator. The residue obtained was purified by preparative HPLC (Method 13). Concentration of the product fractions and drying under high vacuum gave 85 mg (43% of theory) of the title compound.

¹H-NMR (400 MHz, DMSO-d₆, δ/ppm): 8.62 (br. s, 1H), 5.19-5.08 (m, 1H), 4.69 (s, 2H), 4.00 (t, 2H), 3.62 (t, 2H), 3.54-4.40 (m, 4H), 3.33-3.29 (m, 2H), 3.24 (s, 3H), 2.90-2.79 (m, 2H), 2.42 (s, 3H).

LC/MS (Method 2, ESIpos): Rₜ=0.80 min, m/z=457 [M+H]⁺.

### Example 129

### 3-(3,3-Difluorocyclobutyl)-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione

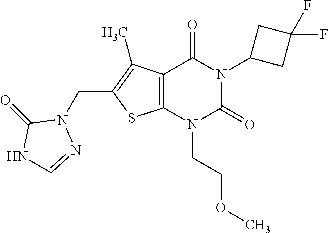 [SMILES: COCCn1c(=O)n(C2CC(F)(F)C2)c(=O)c2c(C)c(Cn3nc[nH]c3=O)sc21]

232 mg (0.39 mmol, 86% purity) of the compound from Ex. 275A were initially charged in 9.5 ml of trimethyl orthoformate and 9.5 ml of methanol, and 1 ml (3.9 mmol) of a 4 M solution of hydrogen chloride in dioxane were added. Since the conversion was still incomplete after stirring at RT for 18 h, a further 0.5 ml of 4 M hydrogen chloride in dioxane was added and the mixture was stirred for a further 4 h. Thereafter, another 0.5 ml of 4 M hydrogen chloride in dioxane was added, and the mixture was stirred for a further 2.5 h. Then water was added to the reaction mixture, which was extracted with ethyl acetate. The organic phase was concentrated and the residue was purified by means of preparative HPLC (Method 13). The product fractions were combined and concentrated, and the residue was dried under high vacuum. 90 mg (55% of theory) of the title compound were obtained.

¹H-NMR (400 MHz, DMSO-d₆, δ/ppm): 11.60 (br. s, 1H), 7.83 (s, 1H), 5.20-5.09 (m, 1H), 4.93 (s, 2H), 3.99 (t, 2H), 3.61 (t, 2H), 3.52-3.39 (m, 2H), 3.23 (s, 3H), 2.90-2.80 (m, 2H), 2.44 (s, 3H).

LC/MS (Method 2, ESIpos): Rₜ=0.77 min, m/z=428 [M+H]⁺.

### Example 130

### 5-Methyl-3-(oxetan-3-yl)-6-[(2-oxoimidazolidin-1-yl)methyl]-1-(3,3,3-trifluoropropyl)thieno[2,3-d]pyrimidine-2,4(1H,3H)-dione

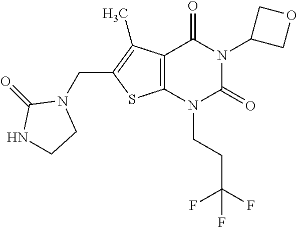 [SMILES: Cc1c(CN2CCNC2=O)sc2c1c(=O)n(C1COC1)c(=O)n2CCC(F)(F)F]

Analogously to the method described in Ex. 3, 135 mg (0.266 mmol, 80% purity) of the compound from Ex. 137A and 52 mg (0.319 mmol) of CDI were used to prepare 49 mg (41% of theory) of the title compound.

¹H-NMR (400 MHz, DMSO-d₆, δ/ppm): 6.54 (s, 1H), 5.02 (quin, 1H), 4.76-4.65 (m, 4H), 4.37 (s, 2H), 4.06 (t, 2H), 3.28-3.18 (m, 4H), 2.81-2.68 (m, 2H), 2.36 (s, 3H).

LC/MS (Method 1, ESIpos): Rₜ=1.24 min, m/z=433.11 [M+H]⁺.

### Example 131

### [1-{[5-Methyl-3-(oxetan-3-yl)-2,4-dioxo-1-(3,3,3-trifluoropropyl)-1,2,3,4-tetrahydrothieno[2,3-d]pyrimidin-6-yl]methyl}imidazolidin-2-ylidene]cyanamide

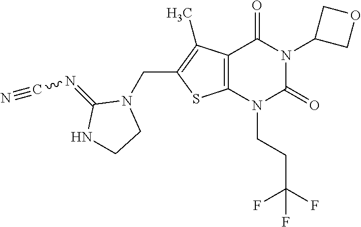 [SMILES: Cc1c(CN2CCNC2=NC#N)sc2c1c(=O)n(C1COC1)c(=O)n2CCC(F)(F)F]

Analogously to the method described in Ex. 41, 135 mg (0.266 mmol, 80% purity) of the compound from Ex. 137A and 58 mg (0.399 mmol) of dimethyl N-cyanodithioiminocarbonate were used to prepare 14 mg (11% of theory) of the title compound.

¹H-NMR (400 MHz, DMSO-d₆, δ/ppm): 8.09 (s, 1H), 5.02 (quin, 1H), 4.73-4.67 (m, 4H), 4.50 (s, 2H), 4.07 (t, 2H), 3.55-3.37 (m, 4H), 2.84-2.67 (m, 2H), 2.38 (s, 3H).

LC/MS (Method 1, ESIpos): Rₜ=1.35 min, m/z=457.12 [M+H]⁺.

### Example 132

### Methyl [1-{[5-methyl-3-(oxetan-3-yl)-2,4-dioxo-1-(3,3,3-trifluoropropyl)-1,2,3,4-tetrahydrothieno[2,3-d]pyrimidin-6-yl]methyl}imidazolidin-2-ylidene]carbamate

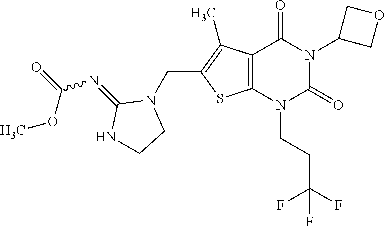 [SMILES: COC(=O)N=C1NCCN1Cc1sc2c(c1C)c(=O)n(C1COC1)c(=O)n2CCC(F)(F)F]

Analogously to the method described in Ex. 87, 135 mg (0.266 mmol, 80% purity) of the compound from Ex. 137A and 83 mg (0.531 mmol) of methyl (dichloromethylene)carbamate were used to prepare 7 mg (5% of theory) of the title compound.

¹H-NMR (400 MHz, DMSO-d₆, δ/ppm): 8.03 (s, 1H), 5.02 (quin, 1H), 4.75-4.65 (m, 4H), 4.58 (s, 2H), 4.05 (t, 2H), 3.53 (s, 3H), 3.50-3.43 (m, 2H), 3.38-3.32 (m, 2H), 2.82-2.64 (m, 2H), 2.39 (s, 3H).

LC/MS (Method 3, ESIpos): Rₜ=1.82 min, m/z=490 [M+H]⁺.

### Example 133

### 6-[(2,3-Dioxopiperazin-1-yl)methyl]-5-methyl-3-(oxetan-3-yl)-1-(3,3,3-trifluoropropyl)thieno[2,3-d]pyrimidine-2,4(1H,3H)-dione

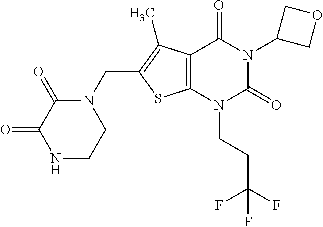 [SMILES: Cc1c(CN2CCNC(=O)C2=O)sc2c1c(=O)n(C1COC1)c(=O)n2CCC(F)(F)F]

Analogously to the method described in Ex. 88, 135 mg (0.266 mmol, 80% purity) of the compound from Ex. 137A and 73 mg (0.492 mmol) of diethyl oxalate were used to prepare 15 mg (12% of theory) of the title compound.

¹H-NMR (400 MHz, DMSO-d₆, δ/ppm): 8.63 (br. s, 1H), 5.02 (quin, 1H), 4.75-4.65 (m, 4H), 4.71 (s, 2H), 4.06 (t, 2H), 3.53-3.45 (m, 2H), 2.83-2.67 (m, 2H), 2.40 (s, 3H).

LC/MS (Method 1, ESIpos): Rₜ=1.08 min, m/z=461.11 [M+H]⁺.

### Example 134

### 5-Methyl-3-(1-methylcyclobutyl)-6-[(2-oxoimidazolidin-1-yl)methyl]-1-(3,3,3-trifluoropropyl)thieno[2,3-d]pyrimidine-2,4(1H,3H)-dione

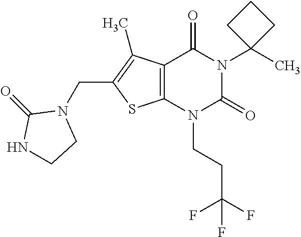 [SMILES: Cc1c(CN2CCNC2=O)sc2c1c(=O)n(C1(C)CCC1)c(=O)n2CCC(F)(F)F]

Analogously to the method described in Ex. 3, 340 mg (0.650 mmol, 80% purity) of the compound from Ex. 138A and 126 mg (0.780 mmol) of CDI were used to prepare 174 mg (60% of theory) of the title compound.

¹H-NMR (400 MHz, DMSO-d₆, δ/ppm): 6.53 (s, 1H), 4.35 (s, 2H), 4.03 (t, 2H), 3.29-3.16 (m, 4H), 2.82-2.64 (m, 2H), 2.36 (s, 3H), 2.34-2.21 (m, 4H), 1.82-1.56 (m, 2H), 1.51 (s, 3H).

LC/MS (Method 1, ESIpos): Rₜ=1.80 min, m/z=445.15 [M+H]⁺.

### Example 135

### [1-{[5-Methyl-3-(1-methylcyclobutyl)-2,4-dioxo-1-(3,3,3-trifluoropropyl)-1,2,3,4-tetrahydrothieno[2,3-d]pyrimidin-6-yl]methyl}imidazolidin-2-ylidene]cyanamide

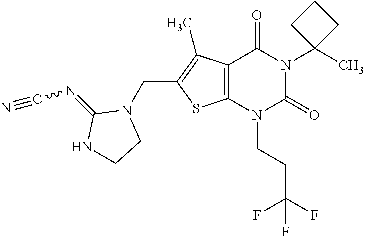 [SMILES: Cc1c(CN2CCNC2=NC#N)sc2c1c(=O)n(C1(C)CCC1)c(=O)n2CCC(F)(F)F]

Analogously to the method described in Ex. 41, 340 mg (0.650 mmol, 80% purity) of the compound from Ex. 138A and 143 mg (0.975 mmol) of dimethyl N-cyanodithioiminocarbonate were used to prepare 140 mg (45% of theory) of the title compound. The preparative HPLC was followed here by another second purification step by means of MPLC (Biotage Isolera One, SNAP KP-Sil cartridge, 50 g of silica gel, eluent: cyclohexane/ethyl acetate 1:2→0:1).

¹H-NMR (400 MHz, DMSO-d₆, δ/ppm): 8.07 (s, 1H), 4.48 (s, 2H), 4.05 (t, 2H), 3.52-3.37 (m, 4H), 2.83-2.65 (m, 2H), 2.38 (s, 3H), 2.33-2.24 (m, 4H), 1.81-1.56 (m, 2H), 1.51 (s, 3H).

LC/MS (Method 1, ESIpos): Rₜ=1.89 min, m/z=469.16 [M+H]⁺.

### Example 136

### Methyl [1-{[5-methyl-3-(1-methylcyclobutyl)-2,4-dioxo-1-(3,3,3-trifluoropropyl)-1,2,3,4-tetrahydrothieno[2,3-d]pyrimidin-6-yl]methyl}imidazolidin-2-ylidene]carbamate

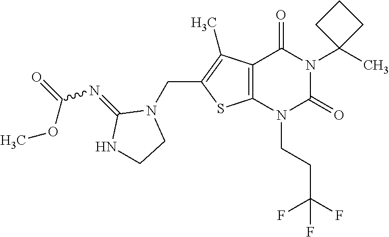

340 mg (0.650 mmol, 80% purity) of the compound from Ex. 138A and 181 μl (1.30 mmol) of triethylamine were dissolved in 15 ml of dichloromethane, and a solution of 203 mg (1.30 mmol) of methyl (dichloromethylene)carbamate in 10 ml of dichloromethane was added dropwise. After the reaction mixture had been stirred at RT for about 18 h, it was concentrated to dryness and the residue was purified by means of preparative HPLC (Method 11). The product fractions were combined and concentrated and the residue was finally stirred with pentane. 84 mg (25% of theory) of the title compound were isolated.

¹H-NMR (400 MHz, DMSO-d₆, δ/ppm): 8.02 (s, 1H), 4.56 (s, 2H), 4.03 (br. t, 2H), 3.53 (s, 3H), 3.49-3.42 (m, 2H), 3.37-3.31 (m, 2H, partially concealed by water signal), 2.80-2.63 (m, 2H), 2.39 (s, 3H), 2.33-2.25 (m, 4H), 1.82-1.56 (m, 2H), 1.51 (s, 3H).

LC/MS (Method 2, ESIpos): Rₜ=0.93 min, m/z=502 [M+H]⁺.

### Example 137

### 6-[(2,3-Dioxopiperazin-1-yl)methyl]-5-methyl-3-(1-methylcyclobutyl)-1-(3,3,3-trifluoropropyl)thieno[2,3-d]pyrimidine-2,4(1H,3H)-dione

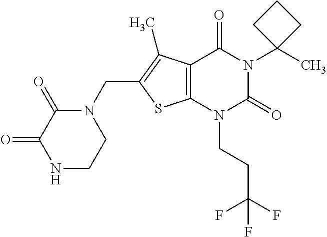 [SMILES: Cc1c(CN2CCNC(=O)C2=O)sc2c1c(=O)n(C1(C)CCC1)c(=O)n2CCC(F)(F)F]

Analogously to the method described in Ex. 88, 340 mg (0.650 mmol, 80% purity) of the compound from Ex. 138A and 178 mg (1.20 mmol) of diethyl oxalate were used to prepare 138 mg (44% of theory) of the title compound. Prior to the final purification by means of preparative HPLC, another purification was conducted here by means of MPLC (Biotage Isolera One, SNAP KP-Sil cartridge, 50 g of silica gel, eluent: cyclohexane/ethyl acetate 2:1-dichloromethane/methanol 10:1).

¹H-NMR (400 MHz, DMSO-d₆, δ/ppm): 8.62 (br. s, 1H), 4.69 (s, 2H), 4.04 (t, 2H), 3.54-3.43 (m, 2H), 3.38-3.26 (m, 2H, partially concealed by water signal), 2.82-2.65 (m, 2H), 2.40 (s, 3H), 2.33-2.23 (m, 4H), 1.83-1.56 (m, 2H), 1.51 (s, 3H).

LC/MS (Method 1, ESIneg): Rₜ=1.61 min, m/z=517.14 [M−H+HCOOH]⁻.

### Example 138

### 5-Methyl-3-(1-methylcyclobutyl)-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

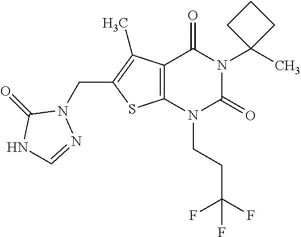 [SMILES: Cc1c(Cn2nc[nH]c2=O)sc2c1c(=O)n(C1(C)CCC1)c(=O)n2CCC(F)(F)F]

Analogously to the method described in Ex. 50, 408 mg (0.650 mmol, 85% purity) of the compound from Ex. 276A were used to prepare 117 mg (40% of theory) of the title compound. The product was purified in this case not by means of preparative HPLC but by means of MPLC (Biotage Isolera One, SNAP KP-Sil cartridge, 100 g of silica gel, eluent: cyclohexane/ethyl acetate 1:2-0:1).

¹H-NMR (400 MHz, DMSO-d₆, δ/ppm): 11.59 (s, 1H), 7.84 (s, 1H), 4.94 (s, 2H), 4.08-3.95 (m, 2H), 2.80-2.64 (m, 2H), 2.42 (s, 3H), 2.34-2.22 (m, 4H), 1.82-1.56 (m, 2H), 1.51 (s, 3H).

LC/MS (Method 2, ESIpos): Rₜ=0.92 min, m/z=444 [M+H]⁺.

### Example 139

### 1-(2-Methoxyethyl)-5-methyl-3-(1-methylcyclobutyl)-6-[(2-oxoimidazolidin-1-yl)methyl]thieno[2,3-d]pyrimidine-2,4(1H,3H)-dione

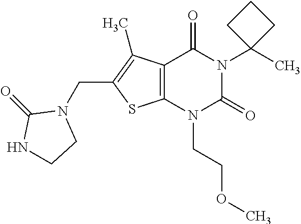 [SMILES: COCCn1c(=O)n(C2(C)CCC2)c(=O)c2c(C)c(CN3CCNC3=O)sc21]

Analogously to the method described in Ex. 1, 315 mg (0.662 mmol, 80% purity) of the compound from Ex. 139A and 129 mg (0.795 mmol) of CDI were used to prepare 208 mg (77% of theory) of the title compound.

¹H-NMR (400 MHz, DMSO-d₆, δ/ppm): 6.51 (s, 1H), 4.33 (s, 2H), 3.95 (t, 2H), 3.60 (t, 2H), 3.28-3.15 (m, 4H), 3.24 (s, 3H), 2.35 (s, 3H), 2.33-2.23 (m, 4H), 1.85-1.56 (m, 2H), 1.51 (s, 3H).

LC/MS (Method 2, ESIpos): Rₜ=0.85 min, m/z=407 [M+H]⁺.

### Example 140

### [1-{[1-(2-Methoxyethyl)-5-methyl-3-(1-methylcyclobutyl)-2,4-dioxo-1,2,3,4-tetrahydrothieno[2,3-d]pyrimidin-6-yl]methyl}imidazolidine-2-ylidene]cyanamide

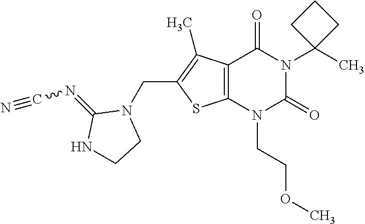 [SMILES: COCCn1c(=O)n(C2(C)CCC2)c(=O)c2c(C)c(CN3CCNC3=NC#N)sc21]

Analogously to the method described in Ex. 41, 315 mg (0.662 mmol, 80% purity) of the compound from Ex. 139A and 145 mg (0.993 mmol) of dimethyl N-cyanodithioiminocarbonate were used to prepare 132 mg (46% of theory) of the title compound. The preparative HPLC was followed here by another second purification step by means of MPLC (Biotage Isolera One, SNAP KP-Sil cartridge, 50 g of silica gel, eluent: cyclohexane/ethyl acetate 1:2-0:1).

¹H-NMR (400 MHz, DMSO-d₆, δ/ppm): 8.06 (s, 1H), 4.46 (s, 2H), 3.97 (br. t, 2H), 3.60 (t, 2H), 3.50-3.36 (m, 4H), 3.25 (s, 3H), 2.36 (s, 3H), 2.33-2.24 (m, 4H), 1.80-1.57 (m, 2H), 1.51 (s, 3H).

LC/MS (Method 1, ESIpos): Rₜ=1.66 min, m/z=431.19 [M+H]⁺.

### Example 141

### Methyl [1-{[1-(2-methoxyethyl)-5-methyl-3-(1-methylcyclobutyl)-2,4-dioxo-1,2,3,4-tetrahydrothieno[2,3-d]pyrimidin-6-yl]methyl}imidazolidine-2-ylidene]carbamate

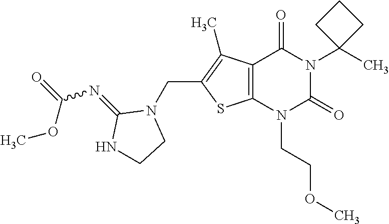

315 mg (0.662 mmol, 80% purity) of the compound from Ex. 139A and 185 μl (1.33 mmol) of triethylamine were dissolved in 10 ml of dichloromethane, and a solution of 207 mg (1.33 mmol) of methyl (dichloromethylene)carbamate in 5 ml of dichloromethane was added dropwise. After the reaction mixture had been stirred at RT for about 18 h, it was concentrated to dryness and the residue was taken up in ethyl acetate. The organic phase was washed successively with water and saturated sodium chloride solution, dried over anhydrous magnesium sulfate, filtered and concentrated. The crude product was purified by preparative HPLC twice (Method 11 each time). The product fractions were combined, concentrated by evaporation and dried under high vacuum. 72 mg (23% of theory) of the title compound were isolated.

¹H-NMR (400 MHz, DMSO-d₆, δ/ppm): 8.02 (s, 1H), 4.54 (s, 2H), 3.94 (t, 2H), 3.59 (t, 2H), 3.53 (s, 3H), 3.49-3.42 (m, 2H), 3.35-3.30 (m, 2H, substantially concealed by water signal), 3.23 (s, 3H), 2.37 (s, 3H), 2.34-2.22 (m, 4H), 1.83-1.56 (m, 2H), 1.51 (s, 3H).

LC/MS (Method 1, ESIpos): Rₜ=1.51 min, m/z=464.20 [M+H]⁺.

### Example 142

### 6-[(2,3-Dioxopiperazin-1-yl)methyl]-1-(2-methoxyethyl)-5-methyl-3-(1-methylcyclobutyl)thieno[2,3-d]pyrimidine-2,4(1H,3H)-dione

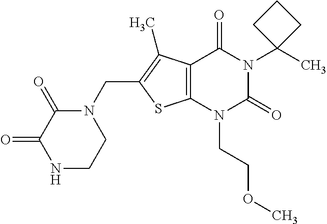 [SMILES: COCCn1c(=O)n(C2(C)CCC2)c(=O)c2c(C)c(CN3CCNC(=O)C3=O)sc21]

Analogously to the method described in Ex. 88, 315 mg (0.662 mmol, 80% purity) of the compound from Ex. 139A and 181 mg (1.20 mmol) of diethyl oxalate were used to prepare 178 mg (60% of theory) of the title compound. Prior to the final purification by means of preparative HPLC, another purification was conducted here by means of MPLC (Biotage Isolera One, SNAP KP-Sil cartridge, 50 of silica gel, eluent: cyclohexane/ethyl acetate 2:1→dichloromethane/methanol 10:1).

¹H-NMR (400 MHz, DMSO-d₆, δ/ppm): 8.61 (br. s, 1H), 4.67 (s, 2H), 3.96 (t, 2H), 3.60 (t, 2H), 3.52-3.42 (m, 2H), 3.33-3.28 (m, 2H, almost entirely concealed by water signal), 3.24 (s, 3H), 2.38 (s, 3H), 2.35-2.22 (m, 4H), 1.81-1.57 (m, 2H), 1.51 (s, 3H).

LC/MS (Method 2, ESIneg): Rₜ=0.76 min, m/z=479 [M−H+HCOOH]⁻.

### Example 143

### 1-(2-Methoxyethyl)-5-methyl-3-(1-methylcyclobutyl)-6-[(5-oxo-4,5-dihydro-H-1,2,4-triazol-1-yl)methyl]thieno[2,3-d]pyrimidine-2,4(1H,3H)-dione

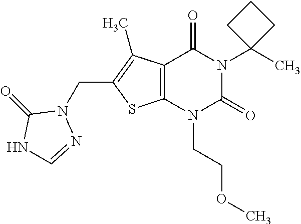 [SMILES: COCCn1c(=O)n(C2(C)CCC2)c(=O)c2c(C)c(Cn3nc[nH]c3=O)sc21]

Analogously to the method described in Ex. 50, 377 mg (0.624 mmol, 82% purity) of the compound from Ex. 277A were used to prepare 45 mg (17% of theory) of the title compound. Prior to the final purification by means of preparative HPLC, another purification was conducted here by means of MPLC (Biotage Isolera One, SNAP KP-Sil cartridge, 100 g of silica gel, eluent: cyclohexane/ethyl acetate 1:2).

¹H-NMR (400 MHz, DMSO-d₆, δ/ppm): 11.58 (br. s, 1H), 7.83 (s, 1H), 4.91 (s, 2H), 3.94 (br. t, 2H), 3.59 (t, 2H), 3.23 (s, 3H), 2.40 (s, 3H), 2.34-2.18 (m, 4H), 1.84-1.55 (m, 2H), 1.51 (s, 3H).

LC/MS (Method 2, ESIpos): Rₜ=0.79 min, m/z=406 [M+H]⁺.

### Example 144

### 3-(trans-3-Methoxycyclobutyl)-5-methyl-6-[(2-oxoimidazolidin-1-yl)methyl]-1-(3,3,3-trifluoropropyl)thieno[2,3-d]pyrimidine-2,4(1H,3H)-dione

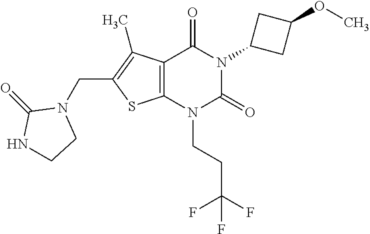 [SMILES: CO[C@H]1C[C@H](n2c(=O)c3c(C)c(CN4CCNC4=O)sc3n(CCC(F)(F)F)c2=O)C1]

400 mg (0.56 mmol, 61% purity) of the compound from Ex. 140A were dissolved in 5 ml of THF, and 109 mg (0.67 mmol) of CDI and 118 μl (0.84 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated sodium hydrogencarbonate solution and saturated sodium chloride solution. The organic phase was then dried over anhydrous magnesium sulfate, filtered and concentrated. The residue was purified by preparative HPLC (Method 13). Concentration of the product fractions and drying under high vacuum gave 160 mg (62% of theory) of the title compound.

¹H-NMR (400 MHz, CD₃OD, δ/ppm): 5.63-5.56 (m, 1H), 4.46 (s, 2H), 4.24-4.21 (m, 2H), 4.16 (dd, 2H), 3.39-3.35 (m, 4H), 3.27 (s, 3H), 3.07-3.01 (m, 2H), 2.74-2.65 (m, 2H), 2.45 (s, 3H), 2.35-2.30 (m, 2H).

LC/MS (Method 2, ESIpos): Rₜ=0.84 min, m/z=461 [M+H]⁺.

### Example 145

### [1-{[3-(trans-3-Methoxycyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

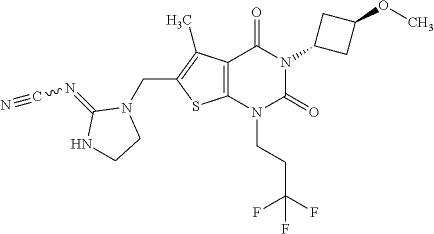 [SMILES: CO[C@H]1C[C@H](n2c(=O)c3c(C)c(CN4CCNC4=NC#N)sc3n(CCC(F)(F)F)c2=O)C1]

507 mg (0.72 mmol, 61% purity) of the compound from Ex. 140A were dissolved in 19 ml of DMF, and 156 mg (1.07 mmol) of dimethyl N-cyanodithioiminocarbonate and 197 mg (1.42 mmol) of potassium carbonate were added. The mixture was stirred in a microwave oven first at RT for 15 min and then at 80° C. for 3 h (Biotage Initiator with dynamic control of irradiation power). Thereafter, the reaction mixture was taken up in ethyl acetate and washed successively with saturated sodium hydrogencarbonate solution, water and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 94 mg (27% of theory) of the title compound.

¹H-NMR (400 MHz, DMSO-d₆, δ/ppm): 5.63-5.56 (m, 1H), 4.55 (s, 2H), 4.26-4.21 (m, 1H), 4.17 (dd, 2H), 3.54 (s, 4H), 3.11 (s, 3H), 3.07-3.01 (m, 2H), 2.75-2.66 (m, 2H), 2.46 (s, 3H), 2.35-2.30 (m, 2H).

LC/MS (Method 1, ESIpos): Rₜ=0.91 min, m/z=485 [M+H]⁺.

### Example 146

### Methyl [1-{[3-(trans-3-methoxycyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

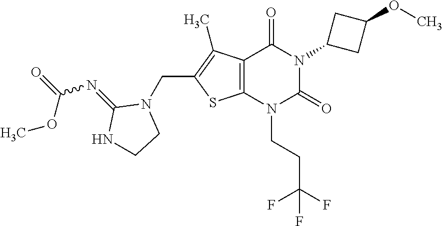

400 mg (0.56 mmol, 61% purity) of the compound from Ex. 140A and 156 μl (1.12 mmol) of triethylamine were dissolved in 25 ml of dichloromethane, and 67 μl (0.56 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 156 mg (54% of theory) of the title compound.

¹H-NMR (400 MHz, CD₃OD, δ/ppm): 5.64-5.55 (m, 1H), 4.80 (s, 2H), 4.23-4.16 (m, 1H), 4.17 (dd, 2H), 3.92 (s, 3H), 3.80 (dd, 2H), 3.66 (dd, 2H), 3.27 (s, 3H), 3.07-3-01 (m, 2H), 2.76-2.37 (m, 2H), 2.48 (s, 3H), 2.36-2.30 (m, 2H).

LC/MS (Method 2, ESIpos): Rₜ=0.84 min, m/z=518 [M+H]⁺.

### Example 147

### 6-[(2,3-Dioxopiperazin-1-yl)methyl]-3-(trans-3-methoxycyclobutyl)-5-methyl-1-(3,3,3-trifluoropropyl)thieno[2,3-d]pyrimidine-2,4(1H,3H)-dione

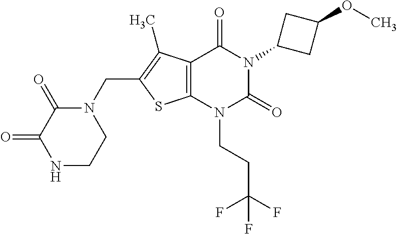 [SMILES: CO[C@H]1C[C@H](n2c(=O)c3c(C)c(CN4CCNC(=O)C4=O)sc3n(CCC(F)(F)F)c2=O)C1]

400 mg (0.56 mmol, 61% purity) of the compound from Ex. 140A were dissolved in 22 ml of ethanol, and 153 mg (1.04 mmol) of diethyl oxalate were added. The mixture was stirred at 80° C. for 2 days. Thereafter, the reaction solution was concentrated on a rotary evaporator. The residue obtained was purified by preparative HPLC (Method 13). Concentration of the product fractions and drying under high vacuum gave 182 mg (66% of theory) of the title compound.

¹H-NMR (400 MHz, CD₃OD, δ/ppm): 5.63-5.56 (m, 1H), 4.78 (s, 2H), 4.24-4.21 (m, 1H), 4.16 (dd, 2H), 3.59 (dd, 2H), 3.46 (dd, 2H), 3.27 (s, 3H), 3.06-3.01 (m, 2H), 2.74-2.65 (m, 2H), 2.49 (s, 3H), 2.36-2.30 (m, 2H).

LC/MS (Method 1, ESIneg): Rₜ=1.38 min, m/z=534 [M−H+HCOOH]⁻.

### Example 148

### 3-(trans-3-Methoxycyclobutyl)-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

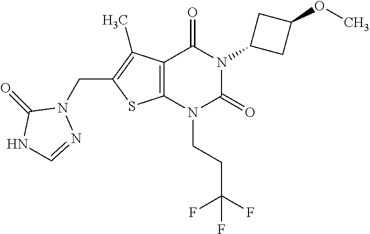 [SMILES: CO[C@H]1C[C@H](n2c(=O)c3c(C)c(Cn4nc[nH]c4=O)sc3n(CCC(F)(F)F)c2=O)C1]

500 mg (0.57 mmol, 62% purity) of the compound from Ex. 278A were initially charged in 14 ml of trimethyl orthoformate and 14 ml of methanol, and 1.4 ml (5.63 mmol) of a 4 M solution of hydrogen chloride in dioxane were added. After stirring for 18 h at RT, a further 0.7 ml of 4 M hydrogen chloride in dioxane was added and the mixture was stirred at RT for a further 3 h. Then water was added to the reaction mixture, which was extracted with ethyl acetate. The organic phase was then dried over anhydrous magnesium sulfate, filtered and concentrated. The residue obtained was purified by preparative HPLC (Method 15). Concentration of the product fractions and drying under high vacuum gave 75 mg (29% of theory) of the title compound.

¹H-NMR (400 MHz, DMSO-d₆, δ/ppm): 11.57 (br. s, 1H), 7.82 (s, 1H), 5.56-5.47 (m, 1H), 4.93 (s, 2H), 4.62 (dd, 2H), 4.09-4.05 (m, 1H), 3.17 (s, 3H), 2.95-2.88 (m, 4H), 2.47 (s, 3H), 2.32-2.28 (m, 2H).

### Example 149

### 3-(trans-3-Methoxycyclobutyl)-1-(2-methoxyethyl)-5-methyl-6-[(2-oxoimidazolidin-1-yl)methyl]thieno[2,3-d]pyrimidine-2,4(1H,3H)-dione

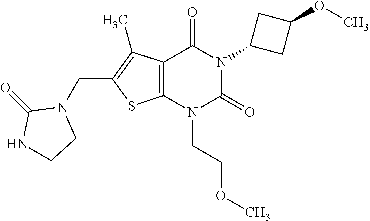 [SMILES: COCCn1c(=O)n([C@H]2C[C@H](OC)C2)c(=O)c2c(C)c(CN3CCNC3=O)sc21]

380 mg (0.61 mmol, 63% purity) of the compound from Ex. 141A were dissolved in 5 ml of THF, and 119 mg (0.73 mmol) of CDI and 128 μl (0.92 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated sodium hydrogencarbonate solution and saturated sodium chloride solution. The organic phase was then dried over anhydrous magnesium sulfate, filtered and concentrated. The residue obtained was purified by preparative HPLC (Method 13). Concentration of the product fractions and drying under high vacuum gave 127 mg (49% of theory) of the title compound.

¹H-NMR (400 MHz, CD₃OD, δ/ppm): 5.64-5.57 (m, 1H), 4.44 (s, 2H), 4.24-4.21 (m, 1H), 4.09 (dd, 2H), 3.70 (dd, 2H), 3.40-3.36 (m, 4H), 3.32 (s, 3H), 3.27 (s, 3H), 3.07-3.01 (m, 2H), 2.44 (s, 3H), 2.35-2.30 (m, 2H).

LC/MS (Method 2, ESIpos): Rₜ=0.71 min, m/z=423 [M+H]⁺.

### Example 150

### [1-{[3-(trans-3-Methoxycyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

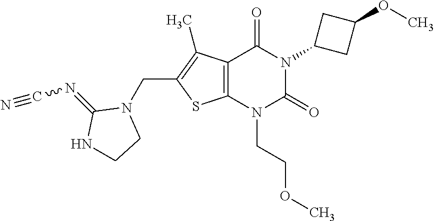 [SMILES: COCCn1c(=O)n([C@H]2C[C@H](OC)C2)c(=O)c2c(C)c(CN3CCNC3=NC#N)sc21]

475 mg (0.76 mmol, 64% purity) of the compound from Ex. 141A were dissolved in 19 ml of DMF, and 168 mg (1.15 mmol) of dimethyl N-cyanodithioiminocarbonate and 211 mg (1.53 mmol) of potassium carbonate were added. The mixture was stirred in a microwave oven first at RT for 15 min and then at 80° C. for 3 h (Biotage Initiator with dynamic control of irradiation power). Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue obtained was purified by means of preparative HPLC (Method 15). Concentration of the product fractions and drying under high vacuum gave 13 mg (4% of theory) of the title compound.

¹H-NMR (400 MHz, DMSO-d₆, δ/ppm): 8.06 (s, 1H), 5.52-5.43 (m, 1H), 4.47 (s, 2H), 4.16-4.11 (m, 1H), 4.00 (dd, 2H), 3.63 (dd, 2H), 3.46-3.39 (m, 4H), 3.24 (s, 3H), 2.96-2.88 (m, 2H), 2.54 (s, 3H), 2.39 (s, 3H), 2.28-2.19 (m, 2H).

LC/MS (Method 6, ESIpos): Rₜ=1.11 min, m/z=447 [M+H]⁺.

### Example 151

### Methyl [1-{[3-(trans-3-methoxycyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]carbamate

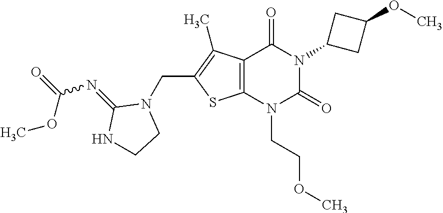

380 mg (0.61 mmol, 64% purity) of the compound from Ex. 141A and 170 μl (1.22 mmol) of triethylamine were dissolved in 28 ml of dichloromethane, and 95 mg (0.61 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 13). Concentration of the product fraction and drying of the residue under high vacuum gave 248 mg (85% of theory) of the title compound.

¹H-NMR (400 MHz, CD₃OD, δ/ppm): 5.64-5.57 (m, 1H), 4.79 (s, 2H), 4.24-4.21 (m, 1H), 4.10 (dd, 2H), 3.92 (s, 3H), 3.80 (dd, 2H), 3.72 (dd, 2H), 3.66 (dd, 2H), 3.32 (s, 3H), 3.27 (s, 3H), 3.07-3.01 (m, 2H), 2.47 (s, 3H), 2.36-2.30 (m, 2H).

LC/MS (Method 2, ESIpos): Rₜ=0.70 min, m/z=481 [M+H]⁺.

### Example 152

### 6-[(2,3-Dioxopiperazin-1-yl)methyl]-3-(trans-3-methoxycyclobutyl)-1-(2-methoxyethyl)-5-methylthieno[2,3-d]pyrimidine-2,4(1H,3H)-dione

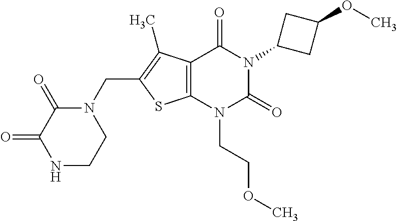 [SMILES: COCCn1c(=O)n([C@H]2C[C@H](OC)C2)c(=O)c2c(C)c(CN3CCNC(=O)C3=O)sc21]

380 mg (0.61 mmol, 63% purity) of the compound from Example 141A were dissolved in 25 ml of ethanol, and 167 mg (1.31 mmol) of diethyl oxalate were added. The mixture was stirred at 80° C. for 2 days. Thereafter, the reaction solution was concentrated on a rotary evaporator. The residue obtained was purified by preparative HPLC (Method 13). Concentration of the product fractions and drying under high vacuum gave 165 mg (57% of theory) of the title compound.

¹H-NMR (400 MHz, CD₃OD, δ/ppm): 5.63-5.56 (m, 1H), 4.78 (s, 2H), 4.24-4.21 (m, 1H), 4.09 (dd, 2H), 3.70 (dd, 2H), 3.59 (dd, 2H), 3.46 (dd, 2H), 3.32 (s, 3H), 3.27 (s, 3H), 3.07-3.01 (m, 2H), 2.48 (s, 3H), 2.35-2.30 (m, 2H).

LC/MS (Method 1, ESIneg): Rₜ=0.88 min, m/z=465 [M−H+HCOOH]⁻.

### Example 153

### 3-(trans-3-Methoxycyclobutyl)-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione

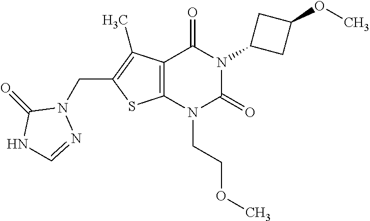 [SMILES: COCCn1c(=O)n([C@H]2C[C@H](OC)C2)c(=O)c2c(C)c(Cn3nc[nH]c3=O)sc21]

500 mg (0.52 mmol, 54% purity) of the compound from Ex. 279A were initially charged in 13 ml of trimethyl orthoformate and 13 ml of methanol, and 1.3 ml (5.20 mmol) of a 4 M solution of hydrogen chloride in dioxane were added. After stirring for 18 h at RT, a further 0.65 ml of 4 M hydrogen chloride in dioxane was added and the mixture was stirred at RT for a further 3 h. Then water was added to the reaction mixture, which was extracted with ethyl acetate. The organic phase was then dried over anhydrous magnesium sulfate, filtered and concentrated. The residue obtained was purified by preparative HPLC (Method 13). Concentration of the product fractions and drying under high vacuum gave 9 mg (4% of theory) of the title compound.

¹H-NMR (400 MHz, DMSO-d₆, δ/ppm): 11.60 (s, 1H), 7.84 (s, 1H), 5.51-5.42 (m, 1H), 4.92 (s, 2H), 4.16-4.10 (m, 1H), 3.98 (dd, 2H), 3.61 (dd, 2H), 3.23 (s, 3H), 3.16 (s, 3H), 2.96-2.88 (m, 2H), 2.43 (s, 2H), 2.26-2.18 (m, 2H).

LC/MS (Method 2, ESIpos): Rₜ=0.67 min, m/z=422 [M+H]⁺.

### Example 154

### 3-(cis-3-Methoxycyclobutyl)-5-methyl-6-[(2-oxoimidazolidin-1-yl)methyl]-1-(3,3,3-trifluoropropyl)thieno[2,3-d]pyrimidine-2,4(1H,3H)-dione

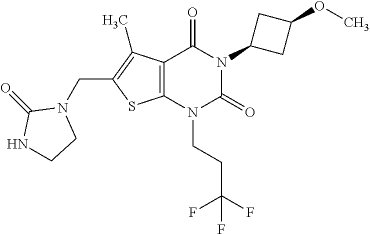 [SMILES: CO[C@H]1C[C@@H](n2c(=O)c3c(C)c(CN4CCNC4=O)sc3n(CCC(F)(F)F)c2=O)C1]

299 mg (0.56 mmol, 81% purity) of the compound from Ex. 142A were dissolved in 4.6 ml of THF, and 108 mg (0.67 mmol) of CDI and 116 μl (0.84 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated sodium hydrogencarbonate solution and saturated sodium chloride solution. The organic phase was then dried over anhydrous magnesium sulfate, filtered and concentrated. The residue obtained was purified by preparative HPLC (Method 15). Concentration of the product fractions and drying under high vacuum gave 55 mg (21% of theory) of the title compound.

¹H-NMR (400 MHz, DMSO-d₆, δ/ppm): 6.53 (s, 1H), 4.71-4.62 (m, 1H), 4.36 (s, 2H), 4.05 (dd, 2H), 3.68-3.31 (m, 1H), 3.28-3.19 (m, 4H), 3.15 (s, 3H), 2.81-2.67 (m, 4H), 2.38 (s, 3H).

LC/MS (Method 2, ESIpos): Rₜ=0.82 min, m/z=461 [M+H]⁺.

### Example 155

### [1-{[3-(cis-3-Methoxycyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

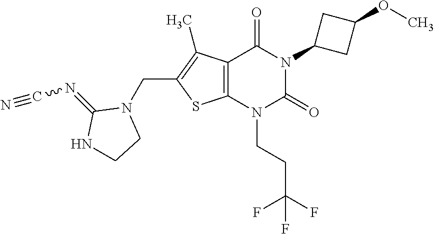 [SMILES: CO[C@H]1C[C@@H](n2c(=O)c3c(C)c(CN4CCNC4=NC#N)sc3n(CCC(F)(F)F)c2=O)C1]

250 mg (0.47 mmol, 81% purity) of the compound from Ex. 142A were dissolved in 12 ml of DMF, and 102 mg (0.70 mmol) of dimethyl N-cyanodithioiminocarbonate and 129 mg (0.93 mmol) of potassium carbonate were added. The mixture was stirred in a microwave oven first at RT for 15 min and then at 80° C. for 3 h (Biotage Initiator with dynamic control of irradiation power). Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue obtained was purified by means of preparative HPLC (Method 15). Concentration of the product fractions and drying under high vacuum gave 38 mg (17% of theory) of the title compound.

¹H-NMR (400 MHz, DMSO-d₆, δ/ppm): 8.08 (s, 1H), 4.69-4.64 (m, 1H), 4.49 (s, 2H), 4.07 (dd, 2H), 3.68-3.61 (m, 1H), 3.47-3.40 (m, 4H), 3.15 (s, 3H), 2.79-2.69 (m, 4H), 2.39 (s, 3H).

LC/MS (Method 2, ESIpos): Rₜ=0.87 min, m/z=485 [M+H]⁺.

### Example 156

### Methyl [1-{[3-(cis-3-methoxycyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

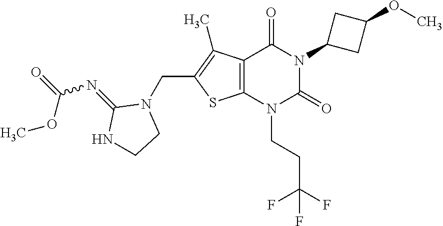

229 mg (0.43 mmol, 81% purity) of the compound from Ex. 142A and 119 μl (0.85 mmol) of triethylamine were dissolved in 20 ml of dichloromethane, and 67 mg (0.43 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The residue obtained was purified by means of preparative HPLC (Method 15). Concentration of the product fractions and drying under high vacuum gave 60 mg (28% of theory) of the title compound.

¹H-NMR (400 MHz, DMSO-d₆, δ/ppm): 8.03 (s, 1H), 4.71-4.62 (m, 1H), 4.57 (s, 2H), 4.04 (dd, 2H), 3.71-3.61 (m, 1H), 3.53 (s, 3H), 3.46 (dd, 2H), 3.34 (dd, 2H), 3.15 (s, 3H), 2.80-2.67 (m, 4H), 2.41 (s, 3H).

LC/MS (Method 2, ESIpos): Rₜ=0.79 min, m/z=518 [M+H]⁺.

### Example 157

### 6-[(2,3-Dioxopiperazin-1-yl)methyl]-3-(cis-3-methoxycyclobutyl)-5-methyl-1-(3,3,3-trifluoropropyl)thieno[2,3-d]pyrimidine-2,4(1H,3H)-dione

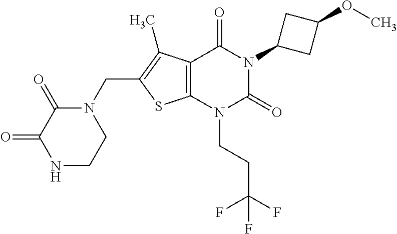 [SMILES: CO[C@H]1C[C@@H](n2c(=O)c3c(C)c(CN4CCNC(=O)C4=O)sc3n(CCC(F)(F)F)c2=O)C1]

249 mg (0.46 mmol, 81% purity) of the compound from Example 142A were dissolved in 19 ml of ethanol, and 127 mg (0.86 mmol) of diethyl oxalate were added. The mixture was stirred at 80° C. for 40 h. Thereafter, the reaction solution was concentrated on a rotary evaporator. The residue obtained was purified by means of preparative HPLC (Method 15). Concentration of the product fractions and drying under high vacuum gave 44 mg (19% of theory) of the title compound.

¹H-NMR (400 MHz, DMSO-d₆, δ/ppm): 8.62 (br. s, 1H), 4.70 (s, 2H), 4.68-4.62 (m, 1H), 4.06 (dd, 2H), 3.68-3.31 (m, 1H), 3.48 (dd, 2H), 3.15 (s, 3H), 2.81-2.67 (m, 4H), 2.42 (s, 3H).

LC/MS (Method 2, ESIpos): Rₜ=0.75 min, m/z=489 [M+H]⁺.

### Example 158

### 3-(cis-3-Methoxycyclobutyl)-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

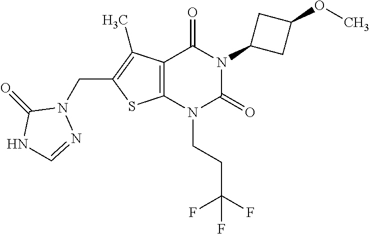 [SMILES: CO[C@H]1C[C@@H](n2c(=O)c3c(C)c(Cn4nc[nH]c4=O)sc3n(CCC(F)(F)F)c2=O)C1]

620 mg (0.60 mmol) of the compound from Ex. 280A were initially charged in 15 ml of trimethyl orthoformate and 15 ml of methanol, and 1.5 ml (6.05 mmol) of a 4 M solution of hydrogen chloride in dioxane were added. Since the conversion was still incomplete after stirring at RT for 18 h, a further 0.75 ml of 4 M hydrogen chloride in dioxane was added and the mixture was stirred for a further 3 h. Then water was added to the reaction mixture, which was extracted with ethyl acetate. The organic phase was concentrated and the residue was purified by means of preparative HPLC (Method 13). The product fractions were combined and concentrated, and the residue was dried under high vacuum. 29 mg (11% of theory) of the title compound were obtained.

¹H-NMR (400 MHz, DMSO-d₆, δ/ppm): 11.60 (s, 1H), 7.84 (s, 1H), 4.94 (s, 2H), 4.69-4.60 (m, 1H), 4.04 (dd, 2H), 3.68-3.60 (m, 1H), 3.15 (s, 3H), 2.79-2.67 (m, 4H), 2.44 (s, 3H).

LC/MS (Method 2, ESIpos): Rₜ=0.78 min, m/z=460 [M+H]⁺.

### Example 159

### 3-(cis-3-Methoxycyclobutyl)-1-(2-methoxyethyl)-5-methyl-6-[(2-oxoimidazolidin-1-yl)methyl]thieno[2,3-d]pyrimidine-2,4(1H,3H)-dione

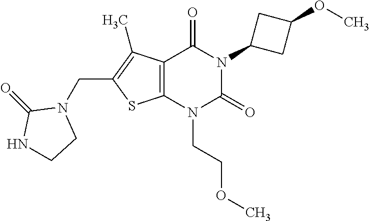 [SMILES: COCCn1c(=O)n([C@H]2C[C@@H](OC)C2)c(=O)c2c(C)c(CN3CCNC3=O)sc21]

250 mg (0.38 mmol, 69% purity) of the compound from Ex. 143A were dissolved in 3 ml of THF, and 74 mg (0.45 mmol) of CDI and 79 μl (0.57 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated aqueous sodium hydrogencarbonate solution and saturated sodium chloride solution. The organic phase was then dried over anhydrous magnesium sulfate, filtered and concentrated. The residue obtained was purified by means of preparative HPLC (Method 15). Concentration of the product fractions and drying under high vacuum gave 16 mg (10% of theory) of the title compound.

¹H-NMR (400 MHz, DMSO-d₆, δ/ppm): 6.52 (s, 1H), 4.73-4.64 (m, 1H), 4.33 (s, 2H), 3.98 (dd, 2H), 3.68-3.60 (m, 3H), 3.24-3.21 (m, 3H), 3.15 (s, 3H), 2.77-2.67 (m, 2H), 2.37 (s, 3H).

LC/MS (Method 2, ESIpos): Rₜ=0.70 min, m/z=423 [M+H]⁺.

### Example 160

### [1-{[3-(cis-3-Methoxycyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

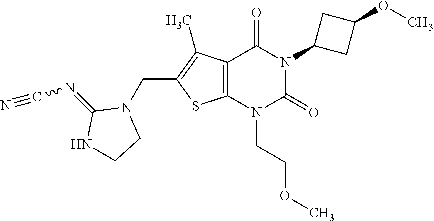 [SMILES: COCCn1c(=O)n([C@H]2C[C@@H](OC)C2)c(=O)c2c(C)c(CN3CCNC3=NC#N)sc21]

250 mg (0.38 mmol, 69% purity) of the compound from Ex. 143A were dissolved in 10 ml of DMF, and 83 mg (0.57 mmol) of dimethyl N-cyanodithioiminocarbonate and 104 mg (0.76 mmol) of potassium carbonate were added. The mixture was stirred in a microwave oven first at RT for 30 min and then at 80° C. for 2 h (Biotage Initiator with dynamic control of irradiation power). Thereafter, the reaction mixture was taken up in ethyl acetate and washed successively with saturated aqueous sodium hydrogencarbonate solution, water and saturated sodium chloride solution. The organic phase was then dried over anhydrous magnesium sulfate, filtered and concentrated. The residue obtained was purified by means of preparative HPLC (Method 15). Concentration of the product fractions and drying under high vacuum gave 21 mg (13% of theory) of the title compound.

¹H-NMR (400 MHz, DMSO-d₆, δ/ppm): 8.07 (s, 1H), 4.72-4.64 (m, 1H), 4.46 (s, 2H), 3.39 (dd, 2H), 3.66-3.61 (m, 3H), 3.48-3.38 (m, 4H), 3.11 (s, 3H), 3.15 (s, 3H), 2.76-2.69 (m, 2H), 2.38 (s, 3H).

LC/MS (Method 2, ESIpos): Rₜ=0.76 min, m/z=447 [M+H]⁺.

### Example 161

### Methyl [1-{[3-(cis-3-methoxycyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]carbamate

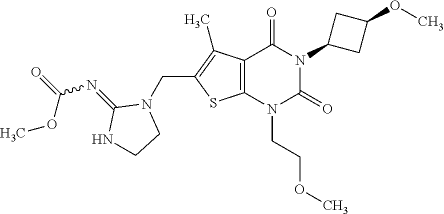

250 mg (0.43 mmol, 69% purity) of the compound from Ex. 143A and 121 μl (0.87 mmol) of triethylamine were dissolved in 20 ml of dichloromethane, and 67 mg (0.43 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The residue obtained was purified by means of preparative HPLC (Method 15). Concentration of the product fractions and drying under high vacuum gave 29 mg (14% of theory) of the title compound.

¹H-NMR (400 MHz, DMSO-d₆, δ/ppm): 8.02 (s, 1H), 4.73-4.64 (m, 1H), 4.55 (s, 2H), 3.97 (dd, 2H), 3.96-3.60 (m, 3H), 3.53 (s, 3H), 3.46 (dd, 2H), 3.33 (dd, 2H), 3.23 (s, 3H), 3.15 (s, 3H), 2.76-2.65 (m, 2H), 2.39 (s, 3H).

LC/MS (Method 2, ESIpos): Rₜ=0.65 min, m/z=480 [M+H]⁺.

### Example 162

### 6-[(2,3-Dioxopiperazin-1-yl)methyl]-3-(cis-3-methoxycyclobutyl)-1-(2-methoxyethyl)-5-methylthieno[2,3-d]pyrimidine-2,4(1H,3H)-dione

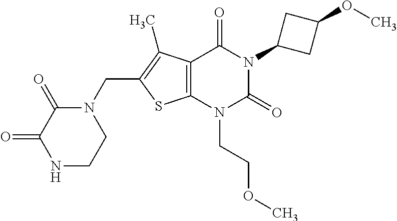 [SMILES: COCCn1c(=O)n([C@H]2C[C@@H](OC)C2)c(=O)c2c(C)c(CN3CCNC(=O)C3=O)sc21]

250 mg (0.43 mmol, 69% purity) of the compound from Example 143A were dissolved in 17 ml of ethanol, and 119 mg (0.81 mmol) of diethyl oxalate were added. The mixture was stirred at 80° C. for 40 h. Thereafter, the reaction solution was concentrated on a rotary evaporator. The residue obtained was purified by means of preparative HPLC (Method 15). Concentration of the product fractions and drying under high vacuum gave 70 mg (36% of theory) of the title compound.

¹H-NMR (400 MHz, DMSO-d₆, δ/ppm): 8.61 (s, 1H), 4.72-4.64 (m, 3H), 3.98 (dd, 2H), 3.68-3.60 (m, 3H), 3.47 (dd, 2H), 3.23 (s, 3H), 3.15 (s, 3H), 2.76-2.67 (m, 2H), 2.40 (s, 3H).

LC/MS (Method 2, ESIpos): Rₜ=0.63 min, m/z=451 [M+H]⁺.

### Example 163

### 3-(cis-3-Methoxycyclobutyl)-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione

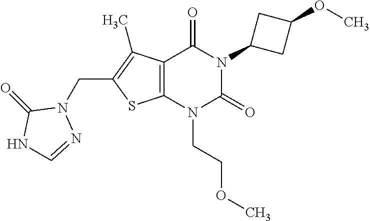 [SMILES: COCCn1c(=O)n([C@H]2C[C@@H](OC)C2)c(=O)c2c(C)c(Cn3nc[nH]c3=O)sc21]

574 mg (0.58 mmol, 59% purity) of the compound from Ex. 281A were initially charged in 14.3 ml of trimethyl orthoformate and 14.3 ml of methanol, and 1.5 ml (5.83 mmol) of a 4 M solution of hydrogen chloride in dioxane were added. After stirring for 48 h at RT, water was added to the reaction mixture. The solids formed were filtered off with suction, washed with acetonitrile and dried under high vacuum. 23 mg (10% of theory) of the title compound were obtained.

¹H-NMR (400 MHz, DMSO-d₆, δ/ppm): 11.59 (s, 1H), 7.84 (s, 1H), 4.92 (s, 2H), 4.71-4.63 (m, 1H), 3.97 (dd, 2H), 3.67-3.59 (m, 3H), 3.23 (s, 3H), 3.15 (s, 3H), 2.75-2.67 (m, 2H), 2.42 (s, 3H).

LC/MS (Method 2, ESIpos): Rₜ=0.63 min, m/z=451 [M+H]⁺.

### Example 164

### 3-(3,3-Dimethylcyclobutyl)-5-methyl-6-[(2-oxoimidazolidin-1-yl)methyl]-1-(3,3,3-trifluoropropyl)thieno[2,3-d]pyrimidine-2,4(1H,3H)-dione

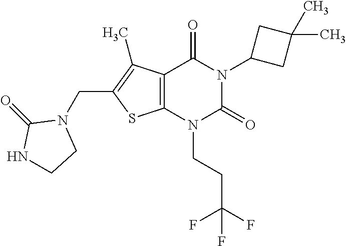 [SMILES: Cc1c(CN2CCNC2=O)sc2c1c(=O)n(C1CC(C)(C)C1)c(=O)n2CCC(F)(F)F]

265 mg (0.56 mmol, 92% purity) of the compound from Ex. 144A were dissolved in 4.6 ml of THF, and 109 mg (0.68 mmol) of CDI and 120 μl (0.84 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated aqueous sodium hydrogencarbonate solution and saturated sodium chloride solution. The organic phase was then dried over anhydrous magnesium sulfate, filtered and concentrated. The residue obtained was purified by preparative HPLC (Method 15). Concentration of the product fractions and drying under high vacuum gave 85 mg (33% of theory) of the title compound.

¹H-NMR (500 MHz, CD₃OD, δ/ppm): 5.30 (quin, 1H), 4.46 (s, 2H), 4.20-4.10 (m, 2H), 3.41-3.34 (m, 4H), 2.79-2.64 (m, 4H), 2.45 (s, 3H), 2.03-1.99 (m, 2H), 1.22 (2s, 6H).

LC/MS (Method 2, ESIpos): Rₜ=1.08 min, m/z=459 [M+H]⁺.

### Example 165

### [1-{[3-(3,3-Dimethylcyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

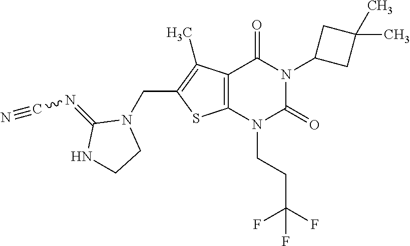 [SMILES: Cc1c(CN2CCNC2=NC#N)sc2c1c(=O)n(C1CC(C)(C)C1)c(=O)n2CCC(F)(F)F]

265 mg (0.56 mmol, 92% purity) of the compound from Ex. 144A were dissolved in 14 ml of DMF, and 124 mg (0.84 mmol) of dimethyl N-cyanodithioiminocarbonate and 156 mg (1.13 mmol) of potassium carbonate were added. The mixture was stirred at 80° C. in a microwave oven (Biotage Initiator with dynamic control of irradiation power) for 3 h. Thereafter, the reaction mixture was taken up in ethyl acetate and washed successively with saturated sodium hydrogencarbonate solution, water and saturated sodium chloride solution. The organic phase was then dried over anhydrous magnesium sulfate, filtered and concentrated. The residue obtained was purified by preparative HPLC (Method 15). Concentration of the product fractions and drying under high vacuum gave 71 mg (25% of theory) of the title compound.

LC/MS (Method 2, ESIpos): Rₜ=1.12 min, m/z=483 [M+H]⁺.

### Example 166

### Methyl [1-{[3-(3,3-dimethylcyclobutyl)-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

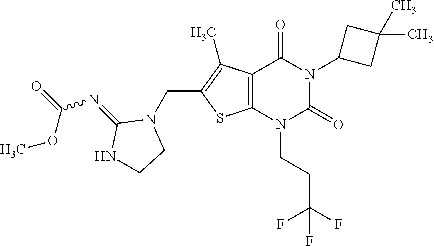

265 mg (0.56 mmol, 92% purity) of the compound from Ex. 144A and 160 μl (1.23 mmol) of triethylamine were dissolved in 26 ml of dichloromethane, and 88 mg (0.56 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 15). Concentration of the product fraction and drying of the residue under high vacuum gave 70 mg (24% of theory) of the title compound.

¹H-NMR (400 MHz, CD₃OD, δ/ppm): 5.33-5.26 (m, 1H), 4.63 (s, 2H), 4.14 (dd, 2H), 3.68 (s, 3H), 3.58 (dd, 2H), 3.42 (dd, 2H), 2.78-2.64 (m, 4H), 2.46 (s, 3H), 2.03-1.99 (m, 2H), 1.22 (2s, 6H).

LC/MS (Method 2, ESIpos): Rₜ=1.06 min, m/z=516 [M+H]⁺.

### Example 167

### 3-(3,3-Dimethylcyclobutyl)-6-[(2,3-dioxopiperazin-1-yl)methyl]-5-methyl-1-(3,3,3-trifluoropropyl)thieno[2,3-d]pyrimidine-2,4(1H,3H)-dione

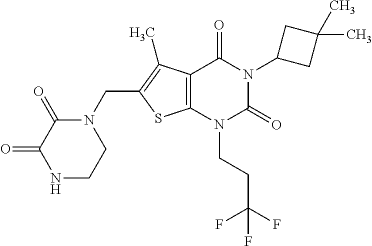 [SMILES: Cc1c(CN2CCNC(=O)C2=O)sc2c1c(=O)n(C1CC(C)(C)C1)c(=O)n2CCC(F)(F)F]

266 mg (0.56 mmol, 92% purity) of the compound from Example 144A were dissolved in 23 ml of ethanol, and 154 mg (0.79 mmol) of diethyl oxalate were added. The mixture was stirred at 80° C. for 3 days. Thereafter, the reaction solution was concentrated on a rotary evaporator. The residue obtained was purified by preparative HPLC (Method 15). Concentration of the product fractions and drying under high vacuum gave 64 mg (23% of theory) of the title compound.

¹H-NMR (400 MHz, CD₃OD, δ/ppm): 5.33-5.25 (m, 1H), 4.79 (s, 2H), 4.15 (dd, 2H), 3.59 (dd, 2H), 3.46 (dd, 2H), 2.78-2.64 (m, 4H), 2.49 (s, 3H), 2.03-1.99 (m, 2H), 1.22 (2s, 6H).

LC/MS (Method 2, ESIpos): Rₜ=0.99 min, m/z=487 [M+H]⁺.

### Example 168

### 3-(3,3-Dimethylcyclobutyl)-5-methyl-6-[(5-oxo-4,5-dihydro-1,2,4-triazol-1-yl)methy]-1-(3,3,3-trifluoropropyl)thieno[2,3-d]pyrimidine-2,4(1H,3H)-dione

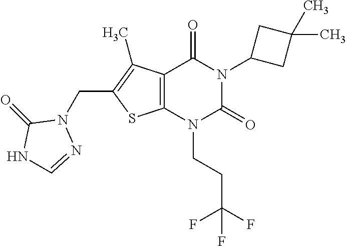 [SMILES: Cc1c(Cn2nc[nH]c2=O)sc2c1c(=O)n(C1CC(C)(C)C1)c(=O)n2CCC(F)(F)F]

449 mg (0.70 mmol, 85% purity) of the compound from Ex. 282A were initially charged in 17 ml of trimethyl orthoformate and 17 ml of methanol, and 1.7 ml (6.96 mmol) of a 4 M solution of hydrogen chloride in dioxane were added. After stirring for 48 h at RT, a further 0.8 ml of 4 M hydrogen chloride in dioxane was added and the mixture was stirred at RT for a further 5 h. Then water was added to the reaction mixture, which was extracted with ethyl acetate. The organic phase was then dried over anhydrous magnesium sulfate, filtered and concentrated. Acetonitrile was added to the residue obtained, and the precipitated solids were filtered off, washed with a little acetonitrile, dried under reduced pressure and finally purified by means of preparative HPLC (Method 13). Concentration of the product fractions and drying under high vacuum gave 107 mg (34% of theory) of the title compound.

¹H-NMR (400 MHz, DMSO-d₆, δ/ppm): 11.59 (s, 1H), 7.64 (s, 1H), 5.25-5.16 (m, 1H), 4.94 (s, 2H), 4.05 (dd, 2H), 2.77-2.64 (m, 4H), 2.45 (s, 3H), 1.96 (dd, 2H), 1.18 (s, 6H).

LC/MS (Method 2, ESIpos): Rₜ=1.02 min, m/z=458 [M+H]⁺.

### Example 169

### 3-(3,3-Dimethylcyclobutyl)-1-(2-methoxyethyl)-5-methyl-6-[(2-oxoimidazolidin-1-yl)methyl]thieno[2,3-d]pyrimidine-2,4(1H,3H)-dione

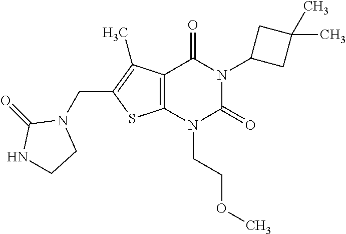 [SMILES: COCCn1c(=O)n(C2CC(C)(C)C2)c(=O)c2c(C)c(CN3CCNC3=O)sc21]

421 mg (0.52 mmol, 48% purity) of the compound from Ex. 145A were dissolved in 4.3 ml of THF, and 101 mg (0.62 mmol) of CDI and 110 μl (0.78 mmol) of triethylamine were added. The mixture was stirred at RT for 16 h. The reaction solution was then concentrated on a rotary evaporator. The residue was taken up in ethyl acetate and washed successively with 1 M hydrochloric acid, saturated sodium hydrogencarbonate solution and saturated sodium chloride solution. The organic phase was then dried over anhydrous magnesium sulfate, filtered and concentrated. The residue obtained was purified by preparative HPLC (Method 15). Concentration of the product fractions and drying under high vacuum gave 10 mg (5% of theory) of the title compound.

¹H-NMR (400 MHz, CD₃OD, δ/ppm): 5.32-5.24 (m, 1H), 4.42 (s, 2H), 4.06 (dd, 2H), 3.68 (dd, 2H), 3.36-3.34 (m, 4H), 3.30 (s, 3H), 2.77-2.73 (m, 2H), 2.41 (s, 3H), 2.00-1.96 (m, 2H), 1.19 (2s, 6H).

LC/MS (Method 2, ESIpos): Rₜ=0.97 min, m/z=421 [M+H]⁺.

### Example 170

### [1-{[3-(3,3-Dimethylcyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

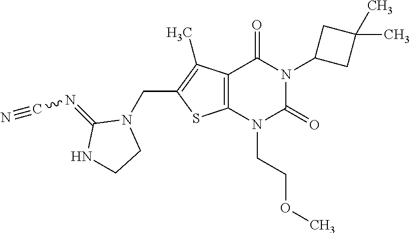 [SMILES: COCCn1c(=O)n(C2CC(C)(C)C2)c(=O)c2c(C)c(CN3CCNC3=NC#N)sc21]

444 mg (0.55 mmol, 49% purity) of the compound from Ex. 145A were dissolved in 14 ml of DMF, and 120 mg (0.82 mmol) of dimethyl N-cyanodithioiminocarbonate and 151 mg (1.09 mmol) of potassium carbonate were added. The mixture was stirred at 80° C. in a microwave oven (Biotage Initiator with dynamic control of irradiation power) for 3.5 h. Thereafter, the reaction mixture was taken up in ethyl acetate and washed successively with saturated sodium hydrogencarbonate solution, water and saturated sodium chloride solution. The organic phase was then dried over anhydrous magnesium sulfate, filtered and concentrated. The residue obtained was purified by preparative HPLC (Method 15). Concentration of the product fractions and drying under high vacuum gave 34 mg (14% of theory) of the title compound.

¹H-NMR (400 MHz, DMSO-d₆, δ/ppm): 8.07 (s, 1H), 5.27-5.18 (m, 1H), 4.46 (s, 2H), 4.00 (t, 2H), 3.62 (t, 2H), 3.46-3.38 (m, 4H), 3.24 (s, 3H), 2.72-2.66 (m, 2H), 2.38 (s, 3H), 1.97-1.92 (m, 2H), 1.18 (2s, 6H).

LC/MS (Method 2, ESIpos): Rₜ=1.02 min, m/z=445 [M+H]⁺.

### Example 171

### Methyl [1-{[3-(3,3-dimethylcyclobutyl)-1-(2-methoxyethyl)-5-methyl-2,4-dioxo-1,2,3,4-tetrahydrothieno[2,3-d]pyrimidin-6-yl]methyl}imidazolidin-2-ylidene]carbamate

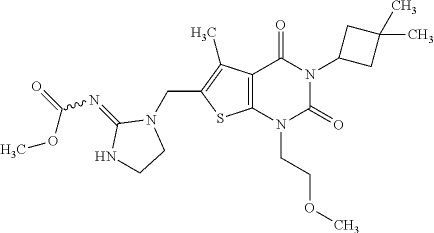

421 mg (0.52 mmol, 48% purity) of the compound from Ex. 145A and 140 μl (1.04 mmol) of triethylamine were dissolved in 24 ml of dichloromethane, and 81 mg (0.52 mmol) of methyl (dichloromethylene)carbamate were added. After the reaction mixture had been stirred at RT for about 16 h, it was diluted with ethyl acetate and washed successively with saturated sodium hydrogencarbonate solution, water and saturated sodium chloride solution. After drying over anhydrous magnesium sulfate, the mixture was filtered and concentrated. The remaining residue was purified by means of preparative HPLC (Method 15). Concentration of the product fraction and drying of the residue under high vacuum gave 41 mg (17% of theory) of the title compound.

¹H-NMR (400 MHz, CD₃OD, δ/ppm): 5.33-5.26 (m, 1H), 4.77 (s, 2H), 4.08 (t, 2H), 3.91 (s, 3H), 3.79 (dd, 2H), 3.70 (t, 2H), 3.67 (dd, 2H), 3.30 (s, 3H), 2.77-2.73 (m, 2H), 2.45 (s, 3H), 2.01-1.97 (m, 2H), 1.22 (2s, 6H).

LC/MS (Method 2, ESIpos): Rₜ=0.93 min, m/z=478 [M+H]⁺.

### Example 172

### 3-(3,3-Dimethylcyclobutyl)-6-[(2,3-dioxopiperazin-1-yl)methyl]-1-(2-methoxyethyl)-5-methylthieno[2,3-d]pyrimidine-2,4(1H,3H)-dione

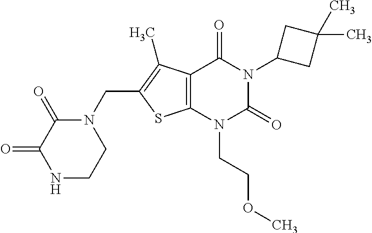 [SMILES: COCCn1c(=O)n(C2CC(C)(C)C2)c(=O)c2c(C)c(CN3CCNC(=O)C3=O)sc21]

444 mg (0.55 mmol, 48% purity) of the compound from Ex. 145A were dissolved in 22 ml of ethanol, and 149 mg (1.01 mmol) of diethyl oxalate were added. The mixture was stirred at 80° C. for 2.5 days. Thereafter, the reaction solution was concentrated on a rotary evaporator. The residue obtained was purified by preparative HPLC (Method 15). Concentration of the product fractions and drying under high vacuum gave 56 mg (23% of theory) of the title compound.

¹H-NMR (400 MHz, DMSO-d₆, δ/ppm): 8.61 (br. s, 1H), 5.27-5.18 (m, 1H), 4.68 (s, 2H), 3.99 (dd, 2H), 3.62 (dd, 2H), 3.48-3.45 (m, 2H), 3.23 (s, 3H), 2.71-2.66 (dd, 2H), 2.41 (s, 3H), 1.97-1.92 (m, 2H), 1.18 (2s, 6H).

LC/MS (Method 2, ESIpos): Rₜ=0.87 min, m/z=449 [M+H]⁺.

### Example 173

### 3-(3,3-Dimethylcyclobutyl)-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione

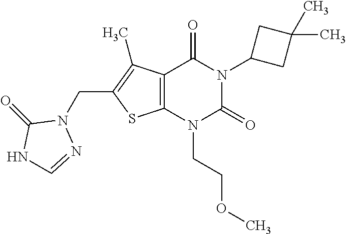 [SMILES: COCCn1c(=O)n(C2CC(C)(C)C2)c(=O)c2c(C)c(Cn3nc[nH]c3=O)sc21]

720 mg (0.834 mmol, 59% purity) of the compound from Ex. 283A were initially charged in 20 ml of trimethyl orthoformate and 20 ml of methanol, and 2.1 ml (8.34 mmol) of a 4 M solution of hydrogen chloride in dioxane were added. After stirring for 48 h at RT, another 1 ml of 4 M hydrogen chloride in dioxane was added and the mixture was stirred at RT for a further 5 h. Then water was added to the reaction mixture, which was extracted with ethyl acetate. The organic phase was then dried over anhydrous magnesium sulfate, filtered and concentrated. Acetonitrile was added to the residue obtained, and the precipitated solids were filtered off, washed with a little acetonitrile, dried under reduced pressure and then purified by means of preparative HPLC (Method 13). Concentration of the product fractions and drying under high vacuum gave 15 mg (4% of theory) of the title compound.

¹H-NMR (400 MHz, DMSO-d₆, δ/ppm): 11.59 (s, 1H), 7.84 (s, 1H), 5.27-5.17 (m, 1H), 4.92 (s, 2H), 3.97 (dd, 2H), 3.61 (dd, 2H), 3.23 (s, 3H), 2.68 (dd, 2H), 2.43 (s, 3H), 1.95 (dd, 2H), 1.18 (2s, 6H).

LC/MS (Method 2, ESIpos): Rₜ=0.90 min, m/z=420 [M+H]⁺.

### Example 174

### 5-Methyl-6-[(2-oxoimidazolidin-1-yl)methyl]-3-(spiro[3.3]hept-2-yl)-1-(3,3,3-trifluoropropyl)thieno[2,3-d]pyrimidine-2,4(1H,3H)-dione

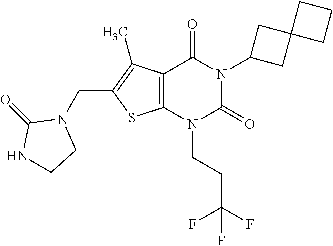 [SMILES: Cc1c(CN2CCNC2=O)sc2c1c(=O)n(C1CC3(CCC3)C1)c(=O)n2CCC(F)(F)F]

Analogously to the method described in Ex. 31, 365 mg (0.673 mmol, 82% purity) of the compound from Ex. 146A and 131 mg (0.808 mmol) of CDI were used to prepare 185 mg (58% of theory) of the title compound.

¹H-NMR (400 MHz, DMSO-d₆, δ/ppm): 6.53 (s, 1H), 5.11-4.98 (m, 1H), 4.35 (s, 2H), 4.04 (t, 2H), 3.28-3.16 (m, 4H), 2.87-2.79 (m, 2H), 2.79-2.69 (m, 2H), 2.37 (s, 3H), 2.23 (td, 2H), 2.10-2.03 (m, 2H), 2.02-1.93 (m, 2H), 1.88-1.75 (m, 2H).

LC/MS (Method 1, ESIpos): Rₜ=2.12 min, m/z=471.17 [M+H]⁺.

### Example 175

### [1-{[5-Methyl-2,4-dioxo-3-(spiro[3.3]hept-2-yl)-1-(3,3,3-trifluoropropyl)-1,2,3,4-tetrahydrothieno[2,3-d]pyrimidin-6-yl]methyl}imidazolidin-2-ylidene]cyanamide

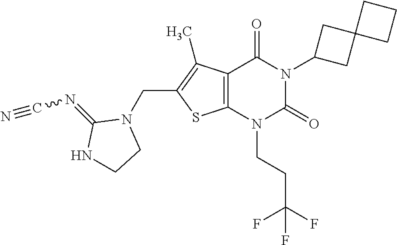 [SMILES: Cc1c(CN2CCNC2=NC#N)sc2c1c(=O)n(C1CC3(CCC3)C1)c(=O)n2CCC(F)(F)F]

365 mg (0.673 mmol, 82% purity) of the compound from Ex. 146A were dissolved in 7 ml of DMF, and 148 mg (1.01 mmol) of dimethyl N-cyanodithioiminocarbonate and 186 mg (1.35 mmol) of potassium carbonate were added. The mixture was stirred at 80° C. in a microwave oven (Biotage Initiator with dynamic control of irradiation power) for 4 h. The reaction mixture was then concentrated to dryness. Stirring of the residue with a little acetonitrile at RT, filtration with suction and drying under high vacuum gave 130 mg (39% of theory) of the title compound.

¹H-NMR (400 MHz, DMSO-d₆, δ/ppm): 8.07 (s, 1H), 5.04 (quin, 1H), 4.48 (s, 2H), 4.06 (br. t, 2H), 3.50-3.36 (m, 4H), 2.87-2.69 (m, 4H), 2.39 (s, 3H), 2.24 (td, 2H), 2.11-2.03 (m, 2H), 2.01-1.94 (m, 2H), 1.87-1.75 (m, 2H).

LC/MS (Method 1, ESIpos): Rₜ=2.18 min, m/z=495.18 [M+H]⁺.

### Example 176

### Methyl [1-{[5-methyl-2,4-dioxo-3-(spiro[3.3]hept-2-yl)-1-(3,3,3-trifluoropropyl)-1,2,3,4-tetrahydrothieno[2,3-d]pyrimidin-6-yl]methyl}imidazolidin-2-ylidene]carbamate

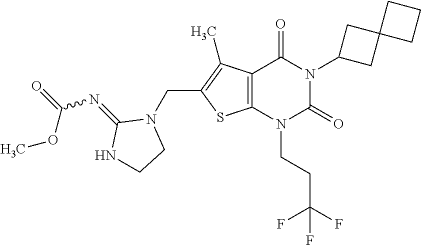 [SMILES: COC(=O)N=C1NCCN1Cc1sc2c(c1C)c(=O)n(C1CC3(CCC3)C1)c(=O)n2CCC(F)(F)F]

365 mg (0.673 mmol, 82% purity) of the compound from Ex. 146A and 188 μl (1.35 mmol) of triethylamine were dissolved in 10 ml of dichloromethane, and a solution of 210 mg (1.35 mmol) of methyl (dichloromethylene)carbamate in 5 ml of dichloromethane was added dropwise. After the reaction mixture had been stirred at RT for about 18 h, it was concentrated to dryness and the residue was taken up in ethyl acetate. The organic phase was washed successively with water and saturated sodium chloride solution, dried over anhydrous magnesium sulfate, filtered and concentrated. The crude product was purified by preparative HPLC (Method 11). The product fractions were combined and concentrated. The residue was stirred with a little acetonitrile at RT, filtered off with suction and dried under high vacuum. 110 mg (30% of theory) of the title compound were isolated.

¹H-NMR (400 MHz, DMSO-d₆, δ/ppm): 8.02 (s, 1H), 5.04 (quin, 1H), 4.56 (s, 2H), 4.04 (br. t, 2H), 3.53 (s, 3H), 3.49-3.40 (m, 2H), 3.37-3.30 (m, 2H, partially concealed by water signal), 2.88-2.79 (m, 2H), 2.73 (dt, 2H), 2.40 (s, 3H), 2.30-2.18 (m, 2H), 2.11-2.02 (m, 2H), 2.01-1.93 (m, 2H), 1.87-1.75 (m, 2H).

LC/MS (Method 2, ESIpos): Rₜ=1.09 min, m/z=528 [M+H]⁺.

### Example 177

### 6-[(2,3-Dioxopiperazin-1-yl)methyl]-5-methyl-3-(spiro[3.3]hept-2-yl)-1-(3,3,3-trifluoropropyl)thieno[2,3-d]pyrimidine-2,4(1H,3H)-dione

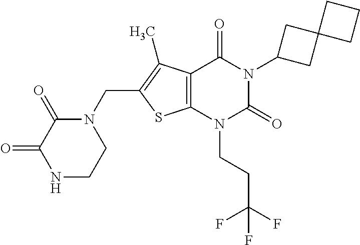 [SMILES: Cc1c(CN2CCNC(=O)C2=O)sc2c1c(=O)n(C1CC3(CCC3)C1)c(=O)n2CCC(F)(F)F]

Analogously to the method described in Ex. 88, 365 mg (0.673 mmol, 82% purity) of the compound from Ex. 146A and 184 mg (1.25 mmol) of diethyl oxalate were used to prepare 184 mg (54% of theory) of the title compound.

¹H-NMR (400 MHz, DMSO-d₆, δ/ppm): 8.64 (br. s, 1H), 5.04 (quin, 1H), 4.70 (s, 2H), 4.05 (t, 2H), 3.53-3.42 (m, 2H), 2.87-2.69 (m, 4H), 2.41 (s, 3H), 2.24 (td, 2H), 2.10-2.03 (m, 2H), 2.01-1.93 (m, 2H), 1.87-1.75 (m, 2H).

LC/MS (Method 1, ESIneg): Rₜ=1.93 min, m/z=543.15 [M−H]⁻.

### Example 178

### 5-Methyl-6-[(5-oxo-4,5-dihydro-1H-1,2,4-triazol-1-yl)methyl]-3-(spiro[3.3]hept-2-yl)-1-(3,3,3-trifluoropropyl)thieno[2,3-d]pyrimidine-2,4(1H,3H)-dione

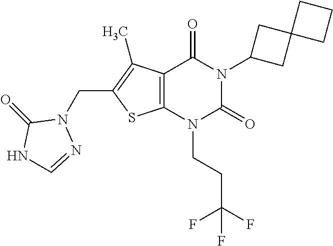 [SMILES: Cc1c(Cn2nc[nH]c2=O)sc2c1c(=O)n(C1CC3(CCC3)C1)c(=O)n2CCC(F)(F)F]

455 mg (0.813 mmol) of the compound from Ex. 284A were dissolved in a mixture of 25 ml each of methanol and trimethyl orthoformate, and 2 ml (8.13 mmol) of a 4 M solution of hydrogen chloride in dioxane were added at RT. After about 16 h, the reaction mixture was concentrated and the residue was taken up in ethyl acetate. The mixture was washed successively with water and saturated sodium chloride solution. After drying over anhydrous magnesium sulfate, the mixture was filtered and concentrated again. The residue that remained was stirred with a little acetonitrile at RT. The solids were filtered off with suction and dried under high vacuum, and gave 153 mg (40% of theory) of the title compound.

¹H-NMR (400 MHz, DMSO-d₆, δ/ppm): 11.59 (br. s, 1H), 7.84 (s, 1H), 5.03 (quin, 1H), 4.94 (s, 2H), 4.03 (t, 2H), 2.86-2.78 (m, 2H), 2.78-2.68 (m, 2H), 2.44 (s, 3H), 2.28-2.19 (m, 2H), 2.11-2.02 (m, 2H), 2.02-1.93 (m, 2H), 1.86-1.75 (m, 2H).

LC/MS (Method 1, ESIpos): Rₜ=1.98 min, m/z=470.15 [M+H]⁺.

### Example 179

### 1-(2-Methoxyethyl)-5-methyl-6-[(2-oxoimidazolidin-1-yl)methyl]-3-(spiro[3.3]hept-2-yl)thieno[2,3-d]pyrimidine-2,4(1H,3H)-dione

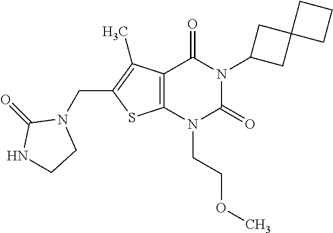 [SMILES: COCCn1c(=O)n(C2CC3(CCC3)C2)c(=O)c2c(C)c(CN3CCNC3=O)sc21]

Analogously to the method described in Ex. 31, 345 mg (0.687 mmol, 81% purity) of the compound from Ex. 147A and 134 mg (0.825 mmol) of CDI were used to prepare 190 mg (63% of theory) of the title compound.

¹H-NMR (400 MHz, DMSO-d₆, δ/ppm): 6.51 (s, 1H), 5.06 (quin, 1H), 4.33 (s, 2H), 3.97 (t, 2H), 3.61 (t, 2H), 3.28-3.15 (m, 4H), 3.23 (s, 3H), 2.91-2.78 (m, 2H), 2.36 (s, 3H), 2.23 (td, 2H), 2.12-2.03 (m, 2H), 2.02-1.93 (m, 2H), 1.87-1.74 (m, 2H).

LC/MS (Method 1, ESIpos): Rₜ=1.90 min, m/z=433.19 [M+H]⁺.

### Example 180

### [1-{[1-(2-Methoxyethyl)-5-methyl-2,4-dioxo-3-(spiro[3.3]hept-2-yl)-1,2,3,4-tetrahydrothieno[2,3-d]pyrimidin-6-yl]methyl}imidazolidin-2-ylidene]cyanamide

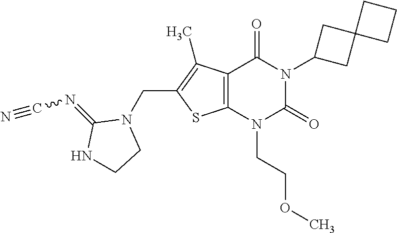 [SMILES: COCCn1c(=O)n(C2CC3(CCC3)C2)c(=O)c2c(C)c(CN3CCNC3=NC#N)sc21]

345 mg (0.687 mmol, 81% purity) of the compound from Ex. 147A were dissolved in 7 ml of DMF, and 151 mg (1.03 mmol) of dimethyl N-cyanodithioiminocarbonate and 190 mg (1.37 mmol) of potassium carbonate were added. The mixture was stirred at 80° C. in a microwave oven (Biotage Initiator with dynamic control of irradiation power) for 4 h. Thereafter, the reaction mixture was diluted with ethyl acetate and washed successively with saturated sodium carbonate solution, water and saturated sodium chloride solution. After drying over anhydrous magnesium sulfate, the mixture was filtered and concentrated. The crude product was stirred with a little acetonitrile at RT. After the solids had been filtered off with suction and dried under high vacuum, 115 mg (36% of theory) of the title compound were obtained.

¹H-NMR (500 MHz, DMSO-d₆, δ/ppm): 8.06 (s, 1H), 5.19-4.94 (m, 1H), 4.46 (s, 2H), 3.98 (t, 2H), 3.61 (t, 2H), 3.51-3.35 (m, 4H), 3.24 (s, 3H), 2.95-2.77 (m, 2H), 2.37 (s, 3H), 2.23 (td, 2H), 2.13-2.02 (m, 2H), 2.01-1.92 (m, 2H), 1.89-1.73 (m, 2H).

LC/MS (Method 2, ESIpos): Rₜ=1.05 min, m/z=457 [M+H]⁺.

### Example 181

### Methyl [1-{[1-(2-methoxyethyl)-5-methyl-2,4-dioxo-3-(spiro[3.3]hept-2-yl)-1,2,3,4-tetrahydrothieno[2,3-d]pyrimidin-6-yl]methyl}imidazolidin-2-ylidene]carbamate

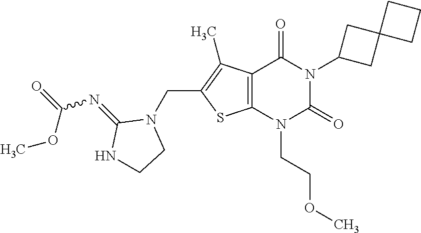 [SMILES: COCCn1c(=O)n(C2CC3(CCC3)C2)c(=O)c2c(C)c(CN3CCNC3=NC(=O)OC)sc21]

Analogously to the method described in Ex. 176, 345 mg (0.687 mmol, 81% purity) of the compound from Ex. 147A and 214 mg (1.37 mmol) of methyl (dichloromethylene)carbamate were used to prepare 166 mg (49% of theory) of the title compound.

¹H-NMR (400 MHz, DMSO-d₆, δ/ppm): 8.02 (s, 1H), 5.14-4.99 (m, 1H), 4.54 (s, 2H), 3.96 (t, 2H), 3.60 (t, 2H), 3.53 (s, 3H), 3.49-3.41 (m, 2H), 3.35-3.29 (m, 2H, partially concealed by water signal), 3.22 (s, 3H), 2.91-2.78 (m, 2H), 2.39 (s, 3H), 2.28-2.17 (m, 2H), 2.10-2.02 (m, 2H), 2.01-1.94 (m, 2H), 1.88-1.75 (m, 2H).

LC/MS (Method 1, ESIpos): Rₜ=1.88 min, m/z=490.21 [M+H]⁺.

### Example 182

### 6-[(2,3-Dioxopiperazin-1-yl)methyl]-1-(2-methoxyethyl)-5-methyl-3-(spiro[3.3]hept-2-yl)thieno[2,3-d]pyrimidine-2,4(1H,3H)-dione

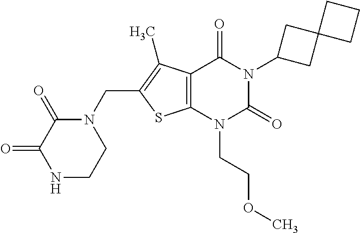 [SMILES: COCCn1c(=O)n(C2CC3(CCC3)C2)c(=O)c2c(C)c(CN3CCNC(=O)C3=O)sc21]

Analogously to the method described in Ex. 88, 345 mg (0.687 mmol, 81% purity) of the compound from Ex. 147A and 188 mg (1.27 mmol) of diethyl oxalate were used to prepare 162 mg (51% of theory) of the title compound.

¹H-NMR (400 MHz, DMSO-d₆, δ/ppm): 8.61 (br. s, 1H), 5.14-4.96 (m, 1H), 4.67 (s, 2H), 3.98 (t, 2H), 3.61 (t, 2H), 3.51-3.43 (m, 2H), 3.23 (s, 3H), 2.90-2.78 (m, 2H), 2.40 (s, 3H), 2.29-2.18 (m, 2H), 2.10-2.03 (m, 2H), 2.02-1.94 (m, 2H), 1.87-1.75 (m, 2H).

LC/MS (Method 1, ESIneg): Rₜ=1.70 min, m/z=505.18 [M−H]⁻.

### Example 183

### 1-(2-Methoxyethyl)-5-methyl-6-[(5-oxo-4,5-dihydro-1H-1,2,4-triazol-1-yl)methyl]-3-(spiro[3.3]hept-2-yl)thieno[2,3-d]pyrimidine-2,4(1H,3H)-dione

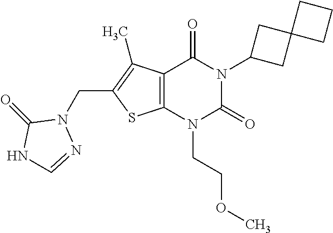 [SMILES: COCCn1c(=O)n(C2CC3(CCC3)C2)c(=O)c2c(C)c(Cn3nc[nH]c3=O)sc21]

375 mg (0.719 mmol) of the compound from Ex. 285A were dissolved in a mixture of 22 ml each of methanol and trimethyl orthoformate, and 1.8 ml (7.19 mmol) of a 4 M solution of hydrogen chloride in dioxane were added at RT. After about 16 h, the reaction mixture was concentrated and the residue was taken up in ethyl acetate. The mixture was washed successively with water and saturated sodium chloride solution. After drying over anhydrous magnesium sulfate, the mixture was filtered and concentrated again. The residue that remained was stirred with a little acetonitrile at RT. The solids were filtered off with suction, then dissolved again in ethyl acetate, and the solution was extracted thoroughly by shaking with water. The organic phase was dried over anhydrous magnesium sulfate, filtered and concentrated. After the residue had been dried under high vacuum, 170 mg (54% of theory) of the title compound were obtained.

¹H-NMR (400 MHz, DMSO-d₆, δ/ppm): 11.58 (br. s, 1H), 7.83 (s, 1H), 5.05 (quin, 1H), 4.91 (s, 2H), 3.96 (br. t, 2H), 3.60 (t, 2H), 3.22 (s, 3H), 2.90-2.75 (m, 2H), 2.42 (s, 3H), 2.23 (td, 2H), 2.10-2.02 (m, 2H), 2.01-1.93 (m, 2H), 1.87-1.74 (m, 2H).

LC/MS (Method 1, ESIpos): Rₜ=1.76 min, m/z=432.17 [M+H]⁺.

### Example 184

### 3-Cyclopentyl-5-methyl-6-[(2-oxoimidazolidin-1-yl)methyl]-1-(3,3,3-trifluoropropyl)thieno[2,3-d]pyrimidine-2,4(1H,3H)-dione

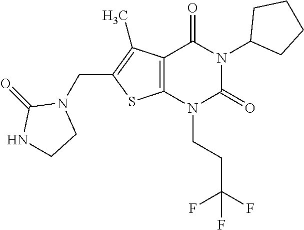 [SMILES: Cc1c(CN2CCNC2=O)sc2c1c(=O)n(C1CCCC1)c(=O)n2CCC(F)(F)F]

To a solution of 240 mg (0.344 mmol, 60% purity) of the compound from Ex. 148A and 72 μl (0.516 mmol) of triethylamine in 10 ml of THF were added 67 mg (0.413 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The remaining residue was purified by means of preparative HPLC (Method 11). After concentration of the product fractions and drying under high vacuum, 42 mg (27% of theory) of the title compound were obtained.

¹H-NMR (500 MHz, DMSO-d₆, δ/ppm): 6.53 (s, 1H), 5.30 (quin, 1H), 4.36 (s, 2H), 4.07 (t, 2H), 3.28-3.16 (m, 4H), 2.83-2.67 (m, 2H), 2.39 (s, 3H), 2.10-1.96 (m, 2H), 1.94-1.83 (m, 2H), 1.78-1.68 (m, 2H), 1.60-1.48 (m, 2H).

LC/MS (Method 2, ESIpos): Rₜ=1.01 min, m/z=445 [M+H]⁺.

### Example 185

### [1-{[3-Cyclopentyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]cyanamide

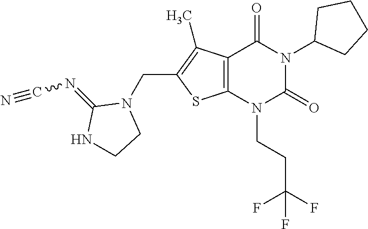 [SMILES: Cc1c(CN2CCNC2=NC#N)sc2c1c(=O)n(C1CCCC1)c(=O)n2CCC(F)(F)F]

249 mg (0.357 mmol, 60% purity) of the compound from Ex. 148A were dissolved in 5 ml of DMF, and 78 mg (0.536 mmol) of dimethyl N-cyanodithioiminocarbonate and 99 mg (0.714 mmol) of potassium carbonate were added. The mixture was stirred at 80° C. in a microwave oven (Biotage Initiator with dynamic control of irradiation power) for 5 h. Thereafter, the reaction mixture was diluted with ethyl acetate and washed successively with water and saturated sodium chloride solution. After drying over anhydrous magnesium sulfate, the mixture was filtered and concentrated. The crude product was purified by preparative HPLC (Method 11). The product fractions were combined, concentrated by evaporation and dried under high vacuum. 34 mg (20% of theory) of the title compound were obtained.

¹H-NMR (500 MHz, DMSO-d₆, δ/ppm): 8.07 (s, 1H), 5.29 (quin, 1H), 4.49 (s, 2H), 4.08 (t, 2H), 3.52-3.36 (m, 4H), 2.84-2.68 (m, 2H), 2.41 (s, 3H), 2.09-1.96 (m, 2H), 1.93-1.84 (m, 2H), 1.81-1.67 (m, 2H), 1.62-1.48 (m, 2H).

LC/MS (Method 2, ESIpos): Rₜ=1.06 min, m/z=469 [M+H]⁺.

### Example 186

### Methyl [1-{[3-cyclopentyl-5-methyl-2,4-dioxo-1-(3,3,3-trifluoropropyl)-1,2,3,4-tetrahydrothieno[2,3-d]pyrimidin-6-yl]methyl}imidazolidin-2-ylidene]carbamate

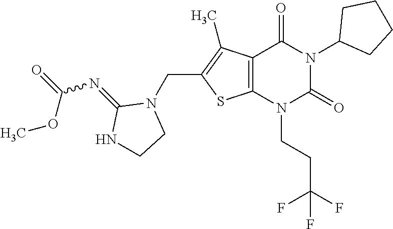 [SMILES: COC(=O)N=C1NCCN1Cc1sc2c(c1C)c(=O)n(C1CCCC1)c(=O)n2CCC(F)(F)F]

248 mg (0.356 mmol, 60% purity) of the compound from Ex. 148A and 99 μl (0.711 mmol) of triethylamine were dissolved in 10 ml of dichloromethane, and a solution of 111 mg (0.711 mmol) of methyl (dichloromethylene)carbamate in 2 ml of dichloromethane was added dropwise. After the reaction mixture had been stirred at RT for about 16 h, it was concentrated to dryness and the residue was taken up in ethyl acetate. The organic phase was washed successively with saturated sodium carbonate solution and saturated sodium chloride solution, dried over anhydrous magnesium sulfate, filtered and concentrated again. The crude product was purified by preparative HPLC (Method 11). The product fractions were combined, concentrated by evaporation and dried under high vacuum. 44 mg (24% of theory) of the title compound were isolated.

¹H-NMR (500 MHz, DMSO-d₆, δ/ppm): 8.02 (s, 1H), 5.29 (quin, 1H), 4.57 (s, 2H), 4.06 (t, 2H), 3.53 (s, 3H), 3.49-3.42 (m, 2H), 3.37-3.31 (m, 2H, partially concealed by water signal), 2.81-2.69 (m, 2H), 2.42 (s, 3H), 2.07-1.97 (m, 2H), 1.94-1.83 (m, 2H), 1.79-1.68 (m, 2H), 1.60-1.48 (m, 2H).

LC/MS (Method 1, ESIpos): Rₜ=1.92 min, m/z=502.17 [M+H]⁺.

### Example 187

### 3-Cyclopentyl-6-[(2,3-dioxopiperazin-1-yl)methyl]-5-methyl-1-(3,3,3-trifluoropropyl)thieno[2,3-d]pyrimidine-2,4(1H,3H)-dione

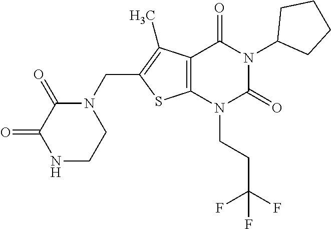 [SMILES: Cc1c(CN2CCNC(=O)C2=O)sc2c1c(=O)n(C1CCCC1)c(=O)n2CCC(F)(F)F]

To a solution of 250 mg (0.358 mmol, 60% purity) of the compound from Ex. 148A in 10 ml of ethanol were added 91 μl (0.663 mmol) of diethyl oxalate, and the mixture was stirred at 80° C. for about 19 h. Then the reaction mixture was concentrated and the residue was taken up in ethyl acetate. The organic phase was washed with saturated sodium chloride solution. After drying over anhydrous magnesium sulfate, the mixture was filtered and concentrated. A crude product was obtained from the residue that remained by means of preparative HPLC (Method 11), and was dissolved once again in ethyl acetate and washed thoroughly with saturated sodium hydrogencarbonate solution. Drying again over anhydrous magnesium sulfate, filtering and concentrating gave 54 mg (31% of theory) of the title compound.

¹H-NMR (400 MHz, DMSO-d₆, δ/ppm): 8.62 (br. s, 1H), 5.29 (quin, 1H), 4.70 (s, 2H), 4.07 (t, 2H), 3.52-3.44 (m, 2H), 3.34-3.29 (m, 2H, almost entirely concealed by water signal), 2.83-2.69 (m, 2H), 2.43 (s, 3H), 2.08-1.96 (m, 2H), 1.95-1.82 (m, 2H), 1.80-1.68 (m, 2H), 1.62-1.49 (m, 2H).

LC/MS (Method 1, ESIneg): Rₜ=1.70 min, m/z=517.14 [M−H]⁻.

### Example 188

### 3-Cyclopentyl-5-methyl-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

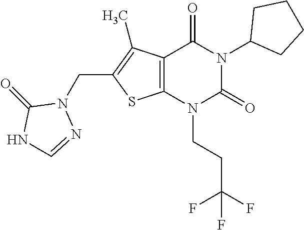 [SMILES: Cc1c(Cn2nc[nH]c2=O)sc2c1c(=O)n(C1CCCC1)c(=O)n2CCC(F)(F)F]

280 mg (0.525 mmol) of the compound from Ex. 286A were dissolved in a mixture of 15 ml each of methanol and trimethyl orthoformate, and 1.3 ml (5.25 mmol) of a 4 M solution of hydrogen chloride in dioxane were added at RT. After about 16 h, a further 656 μl (5.25 mmol) of the 4 M solution of hydrogen chloride in dioxane were added. After a further 2 h, the reaction mixture was concentrated and the residue was taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 116 mg (50% of theory) of the title compound were obtained.

¹H-NMR (400 MHz, DMSO-d₆, δ/ppm): 11.60 (br. s, 1H), 7.84 (s, 1H), 5.29 (quin, 1H), 4.95 (s, 2H), 4.06 (t, 2H), 2.82-2.67 (m, 2H), 2.46 (s, 3H), 2.07-1.96 (m, 2H), 1.95-1.82 (m, 2H), 1.79-1.67 (m, 2H), 1.61-1.47 (m, 2H).

LC/MS (Method 2, ESIpos): Rₜ=0.94 min, m/z=444 [M+H]⁺.

### Example 189

### 3-Cyclopentyl-1-(2-methoxyethyl)-5-methyl-6-[(2-oxoimidazolidin-1-yl)methyl]thieno[2,3-d]pyrimidine-2,4(1H,3H)-dione

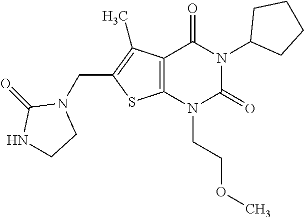 [SMILES: COCCn1c(=O)n(C2CCCC2)c(=O)c2c(C)c(CN3CCNC3=O)sc21]

To a solution of 479 mg (1.03 mmol, 82% purity) of the compound from Ex. 149A and 216 μl (1.55 mmol) of triethylamine in 20 ml of THF were added 201 mg (1.24 mmol) of CDI, and the mixture was stirred at RT for about 16 h. Subsequently, the mixture was concentrated to dryness. The remaining residue was taken up in ethyl acetate and washed successively with 1 M hydrochloric acid, water, saturated sodium hydrogencarbonate solution and saturated sodium chloride solution. After drying over anhydrous magnesium sulfate, the mixture was filtered and concentrated. The solid residue was stirred with a little diisopropyl ether at RT, filtered off with suction and then purified by means of preparative HPLC (Method 11). After concentration of the product fractions and drying under high vacuum, 231 mg (55% of theory) of the title compound were obtained in this way.

¹H-NMR (400 MHz, DMSO-d₆, δ/ppm): 6.52 (s, 1H), 5.30 (quin, 1H), 4.34 (s, 2H), 3.99 (t, 2H), 3.62 (t, 2H), 3.28-3.15 (m, 4H), 3.24 (s, 3H), 2.38 (s, 3H), 2.09-1.96 (m, 2H), 1.95-1.81 (m, 2H), 1.81-1.66 (m, 2H), 1.62-1.48 (m, 2H).

LC/MS (Method 1, ESIpos): Rₜ=1.64 min, m/z=407.17 [M+H]⁺.

### Example 190

### [1-{[3-Cyclopentyl-1-(2-methoxyethyl)-5-methyl-2,4-dioxo-1,2,3,4-tetrahydrothieno[2,3-d]pyrimidin-6-yl]methyl}imidazolidin-2-ylidene]cyanamide

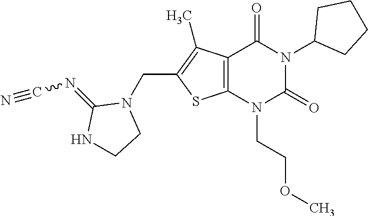 [SMILES: COCCn1c(=O)n(C2CCCC2)c(=O)c2c(C)c(CN3CCNC3=NC#N)sc21]

421 mg (0.907 mmol, 82% purity) of the compound from Ex. 149A were dissolved in 13 ml of DMF, and 199 mg (1.36 mmol) of dimethyl N-cyanodithioiminocarbonate and 251 mg (1.81 mmol) of potassium carbonate were added. The mixture was stirred at 80° C. in a microwave oven (Biotage Initiator with dynamic control of irradiation power) for 5 h. Thereafter, the reaction mixture was diluted with ethyl acetate and washed successively with water and saturated sodium chloride solution. After drying over anhydrous magnesium sulfate, the mixture was filtered and concentrated. The crude product was stirred with a little ethyl acetate at RT, filtered off with suction and then purified by preparative HPLC (Method 11). The product fractions were combined and concentrated and finally stirred at RT with a mixture of 15 ml of pentane and 0.5 ml of dichloromethane. After another filtration with suction and drying under high vacuum, 143 mg (36% of theory) of the title compound were obtained.

¹H-NMR (400 MHz, DMSO-d₆, δ/ppm): 8.06 (s, 1H), 5.30 (quin, 1H), 4.47 (s, 2H), 4.00 (t, 2H), 3.63 (t, 2H), 3.50-3.36 (m, 4H), 3.24 (s, 3H), 2.39 (s, 3H), 2.09-1.96 (m, 2H), 1.95-1.82 (m, 2H), 1.80-1.68 (m, 2H), 1.61-1.48 (m, 2H).

LC/MS (Method 1, ESIpos): Rₜ=1.76 min, m/z=431.19 [M+H]⁺.

### Example 191

### Methyl [1-{[3-cyclopentyl-1-(2-methoxyethyl)-5-methyl-2,4-dioxo-1,2,3,4-tetrahydrothieno[2,3-d]pyrimidin-6-yl]methyl}imidazolidin-2-ylidene]carbamate

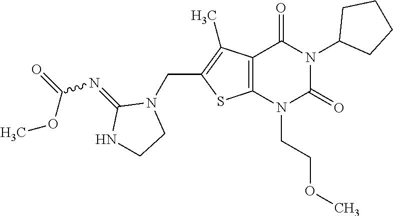 [SMILES: COCCn1c(=O)n(C2CCCC2)c(=O)c2c(C)c(CN3CCNC3=NC(=O)OC)sc21]

Analogously to the method described in Ex. 87, 453 mg (0.976 mmol, 82% purity) of the compound from Ex. 149A and 305 mg (1.95 mmol) of methyl (dichloromethylene)carbamate were used to prepare 142 mg (31% of theory) of the title compound. The product here, after the preparative HPLC purification, was stirred again in a small volume of DMSO at RT.

¹H-NMR (400 MHz, DMSO-d₆, δ/ppm): 8.02 (s, 1H), 5.29 (quin, 1H), 4.55 (s, 2H), 3.98 (t, 2H), 3.61 (t, 2H), 3.53 (s, 3H), 3.49-3.41 (m, 2H), 3.36-3.29 (m, 2H, partially concealed by water signal), 2.41 (s, 3H), 2.09-1.96 (m, 2H), 1.95-1.81 (m, 2H), 1.80-1.66 (m, 2H), 1.62-1.47 (m, 2H).

LC/MS (Method 2, ESIpos): Rₜ=0.84 min, m/z=464 [M+H]⁺.

### Example 192

### 3-Cyclopentyl-6-[(2,3-dioxopiperazin-1-yl)methyl]-1-(2-methoxyethyl)-5-methylthieno[2,3-d]pyrimidine-2,4(1H,3H)-dione

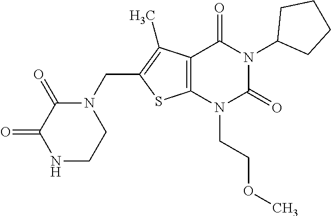 [SMILES: COCCn1c(=O)n(C2CCCC2)c(=O)c2c(C)c(CN3CCNC(=O)C3=O)sc21]

Analogously to the method described in Ex. 88, 448 mg (0.965 mmol, 82% purity) of the compound from Ex. 149A and 264 mg (1.79 mmol) of diethyl oxalate were used to prepare 148 mg (35% of theory) of the title compound. Prior to the purification by means of preparative HPLC (twice here), another first purification step was conducted by means of MPLC (Biotage Isolera One, SNAP KP-Sil cartridge, 25 g of silica gel, eluent: cyclohexane/ethyl acetate 1:1→dichloromethane/methanol 10:1).

¹H-NMR (400 MHz, DMSO-d₆, δ/ppm): 8.61 (br. s, 1H), 5.30 (quin, 1H), 4.68 (s, 2H), 4.00 (t, 2H), 3.62 (t, 2H), 3.52-3.43 (m, 2H), 3.36-3.27 (m, 2H, partially concealed by water signal), 2.42 (s, 3H), 2.09-1.96 (m, 2H), 1.95-1.82 (m, 2H), 1.81-1.68 (m, 2H), 1.62-1.48 (m, 2H).

LC/MS (Method 2, ESIneg): Rₜ=0.79 min, m/z=479 [M−H+HCOOH]⁻.

### Example 193

### 3-Cyclopentyl-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione

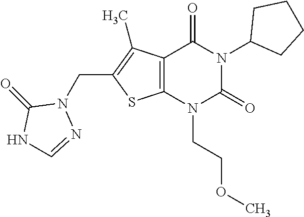 [SMILES: COCCn1c(=O)n(C2CCCC2)c(=O)c2c(C)c(Cn3nc[nH]c3=O)sc21]

Analogously to the method described in Ex. 188, 460 mg (0.845 mmol, 91% purity) of the compound from Ex. 287A were used to prepare 108 mg (31% of theory) of the title compound.

¹H-NMR (400 MHz, DMSO-d₆, δ/ppm): 11.59 (br. s, 1H), 7.84 (s, 1H), 5.29 (quin, 1H), 4.92 (s, 2H), 3.98 (t, 2H), 3.61 (t, 2H), 3.23 (s, 3H), 2.44 (s, 3H), 2.07-1.96 (m, 2H), 1.94-1.82 (m, 2H), 1.80-1.67 (m, 2H), 1.62-1.48 (m, 2H).

LC/MS (Method 1, ESIpos): Rₜ=1.51 min, m/z=406.15 [M+H]⁺.

### Example 194

### 3-Cyclopropyl-1-ethyl-5-methyl-6-[(5-oxo-4,5-dihydro-1H-1,2,4-triazol-1-yl)methyl]thieno[2,3-d]pyrimidine-2,4(1H,3H)-dione

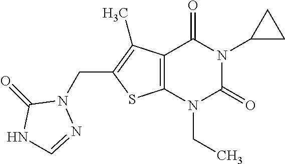 [SMILES: CCn1c(=O)n(C2CC2)c(=O)c2c(C)c(Cn3nc[nH]c3=O)sc21]

170 mg (0.276 mmol, 71% purity) of the compound from Ex. 435A were dissolved in a mixture of 7 ml each of methanol and trimethyl orthoformate, and 690 μl (2.76 mmol) of a 4 M solution of hydrogen chloride in dioxane were added at RT. After 2 days, the reaction mixture was concentrated and the remaining residue was purified by means of preparative HPLC twice (first by Method 11, then by Method 13). After concentration of the product fractions and drying under high vacuum, 40 mg (41% of theory) of the title compound were obtained.

¹H-NMR (400 MHz, DMSO-d₆, δ/ppm): 11.59 (br. s, 1H), 7.83 (s, 1H), 4.93 (s, 2H), 3.84 (q, 2H), 2.62-2.55 (m, 1H), 2.44 (s, 3H), 1.20 (t, 3H), 1.06-0.93 (m, 2H), 0.71-0.61 (m, 2H).

LC/MS (Method 1, ESIpos): Rₜ=1.05 min, m/z=348.11 [M+H]⁺.

### Example 195

### 3-Cyclopropyl-5-methyl-6-[(5-oxo-4,5-dihydro-1H-1,2,4-triazol-1-yl)methyl]-1-propylthieno[2,3-d]pyrimidine-2,4(1H,3H)-dione

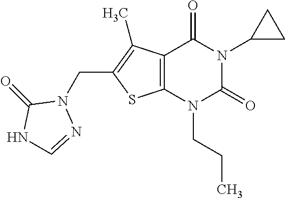 [SMILES: CCCn1c(=O)n(C2CC2)c(=O)c2c(C)c(Cn3nc[nH]c3=O)sc21]

108 mg (0.227 mmol, 95% purity) of the compound from Ex. 436A were dissolved in a mixture of 5.6 ml each of methanol and trimethyl orthoformate, and 568 μl (2.27 mmol) of a 4 M solution of hydrogen chloride in dioxane were added at RT. After 2 days, the reaction mixture was concentrated and the remaining residue was purified by means of preparative HPLC (Method 13). After concentration of the product fractions and drying under high vacuum, 55 mg (66% of theory) of the title compound were obtained.

¹H-NMR (400 MHz, DMSO-d₆, δ/ppm): 11.57 (br. s, 1H), 7.83 (s, 1H), 4.92 (s, 2H), 3.82-3.72 (m, 2H), 2.59 (tt, 1H), 2.44 (s, 3H), 1.66 (sext, 2H), 1.04-0.95 (m, 2H), 0.89 (t, 3H), 0.71-0.62 (m, 2H).

LC/MS (Method 1, ESIpos): Rₜ=1.19 min, m/z=362.13 [M+H]⁺.

### Example 196

### 3-Cyclopropyl-1-(2-fluoroethyl)-5-methyl-6-[(5-oxo-4,5-dihydro-1H-1,2,4-triazol-1-yl)methyl]thieno[2,3-d]pyrimidine-2,4(1H,3H)-dione

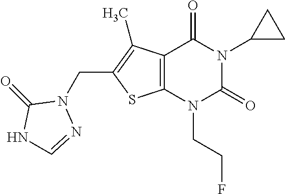 [SMILES: Cc1c(Cn2nc[nH]c2=O)sc2c1c(=O)n(C1CC1)c(=O)n2CCF]

Analogously to the method described in Ex. 195, 71 mg (0.137 mmol, 88% purity) of the compound from Ex. 437A were used to prepare 30 mg (59% of theory) of the title compound.

¹H-NMR (400 MHz, DMSO-d₆, δ/ppm): 11.58 (br. s, 1H), 7.82 (s, 1H), 4.92 (s, 2H), 4.70 (dt, 2H), 4.13 (dt, 2H), 2.63-2.56 (m, 1H), 2.43 (s, 3H), 1.04-0.95 (m, 2H), 0.75-0.61 (m, 2H).

LC/MS (Method 2, ESIpos): Rₜ=0.58 min, m/z=366 [M+H]⁺.

### Example 197

### 3-Cyclopropyl-1-(3-fluoropropyl)-5-methyl-6-[(5-oxo-4,5-dihydro-1H-1,2,4-triazol-1-yl)methyl]thieno[2,3-d]pyrimidine-2,4(1H,3H)-dione

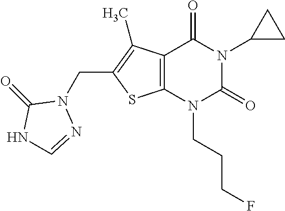 [SMILES: Cc1c(Cn2nc[nH]c2=O)sc2c1c(=O)n(C1CC1)c(=O)n2CCCF]

Analogously to the method described in Ex. 195, 144 mg (0.307 mmol) of the compound from Ex. 438A were used to prepare 74 mg (63% of theory) of the title compound.

¹H-NMR (400 MHz, DMSO-d₆, δ/ppm): 11.59 (br. s, 1H), 7.82 (s, 1H), 4.92 (s, 2H), 4.52 (dt, 2H), 3.92 (t, 2H), 2.62-2.55 (m, 1H), 2.44 (s, 3H), 2.12-1.94 (m, 2H), 1.04-0.95 (m, 2H), 0.70-0.61 (m, 2H).

LC/MS (Method 1, ESIpos): Rₜ=1.09 min, m/z=380.12 [M+H]⁺.

### Example 198

### 3-Cyclopropyl-1-(4-fluorobutyl)-5-methyl-6-[(5-oxo-4,5-dihydro-H-1,2,4-triazol-1-yl)methyl]thieno[2,3-d]pyrimidine-2,4(1H,3H)-dione

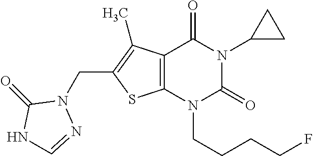 [SMILES: Cc1c(Cn2nc[nH]c2=O)sc2c1c(=O)n(C1CC1)c(=O)n2CCCCF]

Analogously to the method described in Ex. 2, 134 mg (0.252 mmol, 91% purity) of the compound from Ex. 439A were used to prepare 60 mg (60% of theory) of the title compound.

¹H-NMR (400 MHz, DMSO-d₆, δ/ppm): 11.58 (br. s, 1H), 7.83 (s, 1H), 4.92 (s, 2H), 4.58-4.32 (m, 2H), 3.85 (br. t, 2H), 2.62-2.55 (m, 1H), 2.44 (s, 3H), 1.80-1.58 (m, 4H), 1.04-0.94 (m, 2H), 0.71-0.62 (m, 2H).

LC/MS (Method 6, ESIpos): Rₜ=0.93 min, m/z=394 [M+H]⁺.

### Example 199

### 3-Cyclopropyl-1-(2,2-difluoroethyl)-5-methyl-6-[(5-oxo-4,5-dihydro-H-1,2,4-triazol-1-yl)methyl]thieno[2,3-d]pyrimidine-2,4(1H,3H)-dione

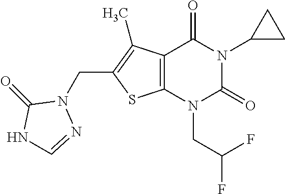 [SMILES: Cc1c(Cn2nc[nH]c2=O)sc2c1c(=O)n(C1CC1)c(=O)n2CC(F)F]

Analogously to the method described in Ex. 195, 102 mg (0.194 mmol, 90% purity) of the compound from Ex. 440A were used to prepare 50 mg (67% of theory) of the title compound.

¹H-NMR (400 MHz, DMSO-d₆, δ/ppm): 11.59 (br. s, 1H), 7.83 (s, 1H), 6.30 (tt, 1H), 4.93 (s, 2H), 4.26 (td, 2H), 2.65-2.56 (m, 1H), 2.44 (s, 3H), 1.08-0.92 (m, 2H), 0.76-0.61 (m, 2H).

LC/MS (Method 1, ESIpos): Rₜ=1.11 min, m/z=384.09 [M+H]⁺.

### Example 200

### 3-Cyclopropyl-5-methyl-6-[(5-oxo-2,5-dihydro-H-pyrazol-1-yl)methyl]-1-(3,3,3-trifluoropropyl)thieno[2,3-d]pyrimidine-2,4(1H,3H)-dione

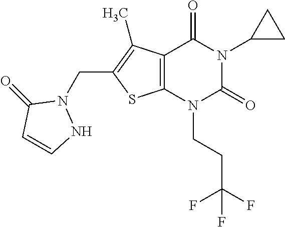 [SMILES: Cc1c(Cn2[nH]ccc2=O)sc2c1c(=O)n(C1CC1)c(=O)n2CCC(F)(F)F]

210 mg (0.337 mmol, 90% purity) of the compound from Ex. 454A were dissolved in 5 ml of dichloromethane and one drop of concentrated sulfuric acid was added. After the reaction mixture had been stirred at RT for 30 min, it was admixed with ice-water. Subsequently, the organic phase was removed and washed with saturated aqueous sodium chloride solution, dried over anhydrous magnesium sulfate, filtered and concentrated. The remaining residue was purified by means of preparative HPLC (Method 11). After concentration of the product fractions and drying under high vacuum, 87 mg (62% of theory) of the title compound were obtained.

¹H-NMR (400 MHz, DMSO-d₆, δ/ppm): 11.29 (broad, 1H), 7.15 (s, 1H), 5.30 (d, 1H), 5.14 (s, 2H), 4.02 (t, 2H), 2.78-2.63 (m, 2H), 2.62-2.56 (m, 1H), 2.46 (s, 3H), 1.06-0.94 (m, 2H), 0.71-0.59 (m, 2H).

LC/MS (Method 1, ESIneg): Rₜ=1.36 min, m/z=413.09 [M−H].

### Example 201

### 3-Cyclopropyl-5-methyl-6-[(5-oxo-4,5-dihydro-1H-1,2,4-triazol-1-yl)methyl]-1-(4,4,4-trifluorobutyl)thieno[2,3-d]pyrimidine-2,4(1H,3H)-dione

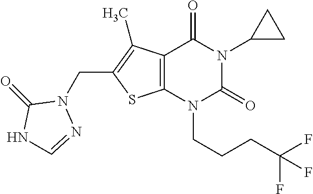 [SMILES: Cc1c(Cn2nc[nH]c2=O)sc2c1c(=O)n(C1CC1)c(=O)n2CCCC(F)(F)F]

Analogously to the method described in Ex. 195, 159 mg (0.291 mmol, 95% purity) of the compound from Ex. 441A were used to prepare 78 mg (62% of theory) of the title compound.

¹H-NMR (400 MHz, DMSO-d₆, δ/ppm): 11.59 (br. s, 1H), 7.82 (s, 1H), 4.93 (s, 2H), 3.90 (t, 2H), 2.62-2.55 (m, 1H), 2.47-2.30 (m, 2H), 2.44 (s, 3H), 1.86 (quin, 2H), 1.05-0.93 (m, 2H), 0.73-0.61 (m, 2H).

LC/MS (Method 1, ESIpos): Rₜ=1.38 min, m/z=430.11 [M+H]⁺.

### Example 202

### 3-Cyclopropyl-1-[(2,2-difluorocyclobutyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 1)

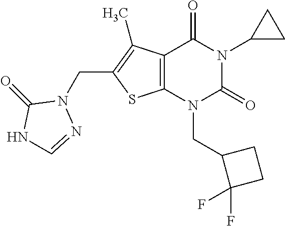 [SMILES: Cc1c(Cn2nc[nH]c2=O)sc2c1c(=O)n(C1CC1)c(=O)n2CC1CCC1(F)F]

Analogously to the method described in Ex. 2, 143 mg (0.278 mmol) of the compound from Ex. 442A were used to prepare 28 mg (23% of theory) of the title compound. The reaction time here was 4 days.

¹H-NMR (400 MHz, DMSO-d₆, δ/ppm): 11.52 (br. s, 1H), 7.83 (br. s, 1H), 4.93 (br. s, 2H), 4.13-3.87 (m, 2H), 2.44 (br. s, 3H), 2.04-1.80 (m, 1H), 1.73-1.48 (m, 1H), 1.12-0.87 (m, 2H), 0.72-0.59 (m, 2H).

LC/MS (Method 2, ESIpos): Rₜ=0.74 min, m/z=424 [M+H]⁺.

### Example 203

### 3-Cyclopropyl-1-[(2,2-difluorocyclobutyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 2)

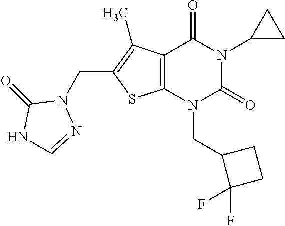 [SMILES: Cc1c(Cn2nc[nH]c2=O)sc2c1c(=O)n(C1CC1)c(=O)n2CC1CCC1(F)F]

Analogously to the method described in Ex. 2, 128 mg (0.199 mmol, 80% purity) of the compound from Ex. 443A were used to prepare 31 mg (36% of theory) of the title compound. The reaction time here was 4 days.

¹H-NMR (400 MHz, DMSO-d₆, δ/ppm): 11.59 (br. s, 1H), 7.83 (s, 1H), 4.93 (s, 2H), 4.10-3.88 (m, 2H), 3.34-3.17 (m, 2H, partially concealed by water signal), 2.63-2.55 (m, 1H), 2.47-2.40 (m, 1H), 2.44 (s, 3H), 1.98-1.83 (m, 1H), 1.68-1.53 (m, 1H), 1.08-0.93 (m, 2H), 0.73-0.58 (m, 2H).

LC/MS (Method 2, ESIpos): Rₜ=0.74 min, m/z=424 [M+H]⁺.

### Example 204

### 3-Cyclopropyl-1-[(3,3-difluorocyclobutyl)methyl]-5-methyl-6-[(2-oxoimidazolidin-1-yl)methyl]thieno[2,3-d]pyrimidine-2,4(1H,3H)-dione

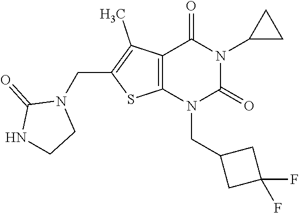 [SMILES: Cc1c(CN2CCNC2=O)sc2c1c(=O)n(C1CC1)c(=O)n2CC1CC(F)(F)C1]

To a solution of 185 mg (0.395 mmol, 85% purity) of the compound from Ex. 361A and 83 μl (0.592 mmol) of triethylamine in 5 ml of THF were added 77 mg (0.474 mmol) of CDI, and the mixture was stirred at RT for about 16 h. Subsequently, the mixture was concentrated to dryness. The remaining residue was taken up in ethyl acetate and washed successively with water and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 86 mg (51% of theory) of the title compound were obtained.

¹H-NMR (400 MHz, DMSO-d₆, δ/ppm): 6.52 (s, 1H), 4.34 (s, 2H), 4.01 (d, 2H), 3.27-3.16 (m, 4H), 2.76-2.41 (m, 6H, partially concealed by DMSO signal), 2.37 (s, 3H), 1.05-0.96 (m, 2H), 0.72-0.61 (m, 2H).

LC/MS (Method 1, ESIpos): Rₜ=1.46 min, m/z=425.14 [M+H]⁺.

### Example 205

### 3-Cyclopropyl-1-[(3,3-difluorocyclobutyl)methyl]-5-methyl-6-[(2-oxo-2,3-dihydro-1H-imidazol-1-yl)methyl]thieno[2,3-d]pyrimidine-2,4(1H,3H)-dione

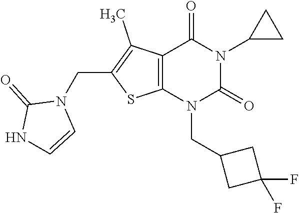 [SMILES: Cc1c(Cn2cc[nH]c2=O)sc2c1c(=O)n(C1CC1)c(=O)n2CC1CC(F)(F)C1]

220 mg (0.375 mmol, 83% purity) of the compound from Ex. 392A were dissolved in 4 ml of methanol, and 0.75 ml of water and 0.75 ml of 1 M hydrochloric acid were added. After the reaction mixture had been stirred at RT for 2 days, it was diluted with water and extracted with ethyl acetate. The organic extract was washed successively with water and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 71 mg (44% of theory) of the title compound were obtained.

¹H-NMR (400 MHz, DMSO-d₆, δ/ppm): 9.98 (br. s, 1H), 6.42 (dd, 1H), 6.34 (t, 1H), 4.78 (s, 2H), 4.00 (d, 2H), 2.74-2.56 (m, 4H), 2.44 (s, 3H), 1.05-0.94 (m, 2H), 0.69-0.61 (m, 2H).

LC/MS (Method 1, ESIpos): Rₜ=1.36 min, m/z=423.13 [M+H]⁺.

### Example 206

### 3-Cyclopropyl-1-[(3,3-difluorocyclobutyl)methyl]-5-methyl-6-[(5-oxo-4,5-dihydro-1H-1,2,4-triazol-1-yl)methyl]thieno[2,3-d]pyrimidine-2,4(1H,3H)-dione

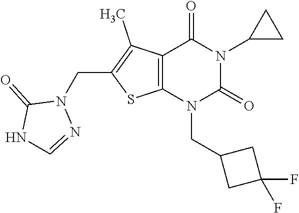 [SMILES: Cc1c(Cn2nc[nH]c2=O)sc2c1c(=O)n(C1CC1)c(=O)n2CC1CC(F)(F)C1]

230 mg (0.448 mmol) of the compound from Ex. 444A were dissolved in a mixture of 15 ml each of methanol and trimethyl orthoformate, and 1.1 ml (4.48 mmol) of a 4 M solution of hydrogen chloride in dioxane were added at RT. After 5 days, the reaction mixture was first concentrated and then taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 103 mg (54% of theory) of the title compound were obtained.

¹H-NMR (400 MHz, DMSO-d₆, δ/ppm): 11.59 (br. s, 1H), 7.83 (s, 1H), 4.92 (s, 2H), 4.00 (d, 2H), 2.75-2.55 (m, 4H), 2.44 (s, 3H), 1.06-0.92 (m, 2H), 0.72-0.59 (m, 2H).

LC/MS (Method 2, ESIpos): Rₜ=0.73 min, m/z=424 [M+H]⁺.

### Example 207

### 3-Cyclopropyl-1-[(2,2-difluorocyclopentyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 1)

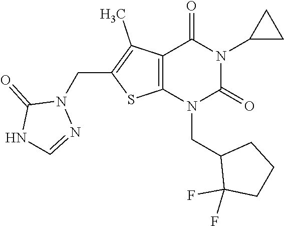 [SMILES: Cc1c(Cn2nc[nH]c2=O)sc2c1c(=O)n(C1CC1)c(=O)n2CC1CCCC1(F)F]

Analogously to the method described in Ex. 2, 128 mg (0.243 mmol) of the compound from Ex. 445A were used to prepare 33 mg (31% of theory) of the title compound.

¹H-NMR (400 MHz, DMSO-d₆, δ/ppm): 11.57 (br. s, 1H), 7.83 (s, 1H), 4.93 (s, 2H), 4.05-3.82 (m, 2H), 2.79-2.63 (m, 1H), 2.59 (tt, 1H), 2.44 (s, 3H), 2.25-1.99 (m, 2H), 1.96-1.83 (m, 1H), 1.82-1.49 (m, 3H), 1.07-0.93 (m, 2H), 0.75-0.56 (m, 2H).

LC/MS (Method 1, ESIpos): Rₜ=1.40 min, m/z=438.14 [M+H]⁺.

### Example 208

### 3-Cyclopropyl-1-[(2,2-difluorocyclopentyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 2)

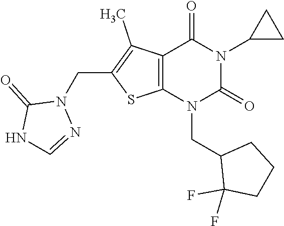 [SMILES: Cc1c(Cn2nc[nH]c2=O)sc2c1c(=O)n(C1CC1)c(=O)n2CC1CCCC1(F)F]

135 mg (0.256 mmol) of the compound from Ex. 446A were dissolved in a mixture of 8 ml each of methanol and trimethyl orthoformate, and 640 μl (2.56 mmol) of a 4 M solution of hydrogen chloride in dioxane were added at RT. After 2 days, the reaction mixture was first concentrated and then taken up in ethyl acetate. The mixture was washed successively with water and saturated sodium chloride solution. After drying over anhydrous magnesium sulfate, the mixture was filtered and concentrated again. The remaining residue was purified by means of preparative HPLC (Method 11). After concentration of the product fractions and drying under high vacuum, 35 mg (31% of theory) of the title compound were obtained.

¹H-NMR (400 MHz, DMSO-d₆, δ/ppm): 11.59 (br. s, 1H), 7.83 (s, 1H), 4.93 (s, 2H), 4.05-3.82 (m, 2H), 2.80-2.63 (m, 1H), 2.59 (tt, 1H), 2.44 (s, 3H), 2.25-2.00 (m, 2H), 1.97-1.84 (m, 1H), 1.81-1.48 (m, 3H), 1.08-0.92 (m, 2H), 0.76-0.54 (m, 2H).

LC/MS (Method 1, ESIpos): Rₜ=1.40 min, m/z=438.14 [M+H]⁺.

### Example 209

### 3-Cyclopropyl-1-[(3,3-difluorocyclopentyl)methyl]-5-methyl-6-[(5-oxo-4,5-dihydro-1H-1,2,4-triazol-1-yl)methyl]thieno[2,3-d]pyrimidine-2,4(1H,3H)-dione (racemate)

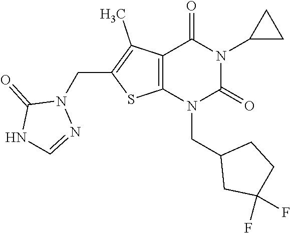 [SMILES: Cc1c(Cn2nc[nH]c2=O)sc2c1c(=O)n(C1CC1)c(=O)n2CC1CCC(F)(F)C1]

Analogously to the method described in Ex. 2, 212 mg (0.402 mmol) of the compound from Ex. 447A were used to prepare 95 mg (54% of theory) of the title compound.

¹H-NMR (400 MHz, DMSO-d₆, δ/ppm): 11.58 (m, 1H), 7.83 (s, 1H), 4.92 (s, 2H), 3.99-3.75 (m, 2H), 2.64-2.55 (m, 2H), 2.44 (s, 3H), 2.31-1.79 (m, 5H), 1.57 (dq, 1H), 1.06-0.91 (m, 2H), 0.72-0.60 (m, 2H).

LC/MS (Method 1, ESIpos): Rₜ=1.42 min, m/z=438.14 [M+H]⁺.

### Example 210

### 3-Cyclopropyl-1-[(3,3-difluorocyclopentyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 1)

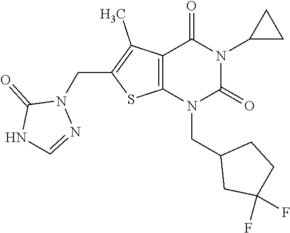 [SMILES: Cc1c(Cn2nc[nH]c2=O)sc2c1c(=O)n(C1CC1)c(=O)n2CC1CCC(F)(F)C1]

90 mg of the racemic compound from Ex. 209 were dissolved in 10 ml of a mixture of ethanol, heptane and dichloromethane, and, in 20 portions, separated into the enantiomers via preparative HPLC on a chiral phase [column: Daicel Chiralpak OD-H 5 μm, 250 mm×20 mm; eluent: n-heptane/ethanol 1:1; flow rate: 25 ml/min; temperature: 40° C.; detection: 220 nm]. After concentration of the product fractions and drying of the solids under high vacuum, 24 mg (53% of theory) of enantiomer 1 were obtained (99% ee, chiral analytical HPLC).

¹H-NMR (400 MHz, DMSO-d₆, δ/ppm): 11.57 (br. s, 1H), 7.82 (s, 1H), 4.92 (s, 2H), 3.96-3.77 (m, 2H), 2.64-2.55 (m, 2H), 2.44 (s, 3H), 2.32-1.80 (m, 5H), 1.57 (dq, 1H), 1.03-0.93 (m, 2H), 0.73-0.57 (m, 2H).

Chiral analytical HPLC [column: Daicel Chiraltek OD-3, 3 μm 50 mm×4.6 mm; eluent: isohexane/ethanol 1:1; flow rate: 3 ml/min; temperature: 40° C.; detection: Rₜ=2.93 min.

LC/MS (Method 2, ESIpos): Rₜ=0.77 min, m/z=438 [M+H]⁺.

### Example 211

### 3-Cyclopropyl-1-[(3,3-difluorocyclopentyl)methyl]-5-methyl-6-[(5-oxo-4,5-dihydro-1H-1,2,4-triazol-1-yl)methyl]thieno[2,3-d]pyrimidine-2,4(1H,3H)-dione (enantiomer 2)

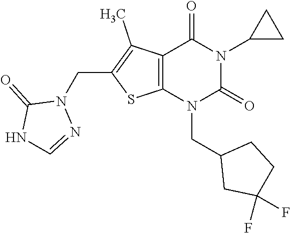 [SMILES: Cc1c(Cn2nc[nH]c2=O)sc2c1c(=O)n(C1CC1)c(=O)n2CC1CCC(F)(F)C1]

27 mg (60% of theory) of enantiomer 2 were obtained from the HPLC separation on a chiral phase described in Ex. 210 (92.6% ee, chiral analytical HPLC).

¹H-NMR (400 MHz, DMSO-d₆, δ/ppm): 11.58 (br. s, 1H), 7.82 (s, 1H), 4.92 (s, 2H), 3.98-3.77 (m, 2H), 2.64-2.55 (m, 2H), 2.44 (s, 3H), 2.32-1.80 (m, 5H), 1.57 (dq, 1H), 1.07-0.91 (m, 2H), 0.74-0.57 (m, 2H).

Chiral analytical HPLC [column: Daicel Chiraltek OD-3, 3 μm 50 mm×4.6 mm; eluent: isohexane/ethanol 1:1; flow rate: 3 ml/min; temperature: 40° C.; detection: 220 nm]: Rₜ=4.00 min.

LC/MS (Method 2, ESIpos): Rₜ=0.77 min, m/z=438 [M+H]⁺.

### Example 212

### 4-{3-Cyclopropyl-5-methyl-2,4-dioxo-6-[(5-oxo-4,5-dihydro-1H-1,2,4-triazol-1-yl)methyl]-3,4-dihydrothieno[2,3-d]pyrimidin-1 (2H)-yl}butanenitrile

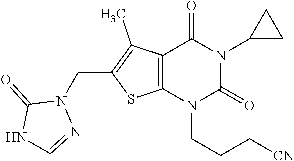 [SMILES: Cc1c(Cn2nc[nH]c2=O)sc2c1c(=O)n(C1CC1)c(=O)n2CCCC#N]

Analogously to the method described in Ex. 195, 140 mg (0.279 mmol, 95% purity) of the compound from Ex. 448A were used to prepare 68 mg (63% of theory) of the title compound.

¹H-NMR (400 MHz, DMSO-d₆, δ/ppm): 11.58 (br. s, 1H), 7.83 (s, 1H), 4.93 (s, 2H), 3.91 (t, 2H), 2.64-2.55 (m, 3H), 2.44 (s, 3H), 1.94 (quin, 2H), 1.06-0.92 (m, 2H), 0.75-0.61 (m, 2H).

LC/MS (Method 6, ESIpos): Rₜ=0.81 min, m/z=387 [M+H]⁺.

### Example 213

### 3-Cyclopropyl-5-methyl-6-[(5-oxo-4,5-dihydro-1H-1,2,4-triazol-1-yl)methyl]-1-{2-[(trifluoromethyl)sulfanyl]ethyl}thieno[2,3-d]pyrimidine-2,4(1H,3H)-dione

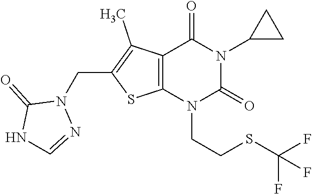 [SMILES: Cc1c(Cn2nc[nH]c2=O)sc2c1c(=O)n(C1CC1)c(=O)n2CCSC(F)(F)F]

Analogously to the method described in Ex. 195, 154 mg (0.215 mmol, 75% purity) of the compound from Ex. 449A were used to prepare 75 mg (78% of theory) of the title compound.

¹H-NMR (400 MHz, DMSO-d₆, δ/ppm): 11.52 (br. s, 1H), 7.83 (s, 1H), 4.93 (s, 2H), 4.08 (t, 2H), 3.27 (t, 2H, very substantially concealed by water signal), 2.63-2.55 (m, 1H), 2.44 (s, 3H), 1.08-0.93 (m, 2H), 0.73-0.59 (m, 2H).

LC/MS (Method 1, ESIpos): Rₜ=1.44 min, m/z=448.07 [M+H]⁺.

### Example 214

### 5-Methyl-3-(1-methylcyclopropyl)-6-[(2-oxo-2,3-dihydro-1H-imidazol-1-yl)methyl]-1-(3,3,3-trifluoropropyl)thieno[2,3-d]pyrimidine-2,4(1H,3H)-dione

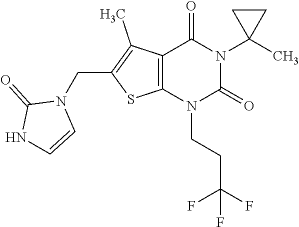 [SMILES: Cc1c(Cn2cc[nH]c2=O)sc2c1c(=O)n(C1(C)CC1)c(=O)n2CCC(F)(F)F]

306 mg (0.621 mmol) of the compound from Ex. 393A were dissolved in 6 ml of methanol, and 1.2 ml of water and 1.2 ml of 1 M hydrochloric acid were added. Since there was still no apparent conversion after stirring at RT overnight, 1 ml of concentrated hydrochloric acid was added and the stirring was continued at RT. After 2 days, the mixture was diluted with water and extracted with ethyl acetate. The organic extract was washed successively with water and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 178 mg (67% of theory) of the title compound were obtained.

¹H-NMR (400 MHz, DMSO-d₆, δ/ppm): 9.99 (br. s, 1H), 6.42 (t, 1H), 6.34 (t, 1H), 4.79 (s, 2H), 4.22-3.84 (m, 2H), 2.81-2.63 (m, 2H), 2.46 (s, 3H), 1.33 (s, 3H), 1.01-0.72 (m, 4H).

LC/MS (Method 1, ESIpos): Rₜ=1.44 min, m/z=429.12 [M+H]⁺.

### Example 215

### 5-Methyl-3-(1-methylcyclopropyl)-6-[(5-oxo-2,5-dihydro-1H-pyrazol-1-yl)methyl]-1-(3,3,3-trifluoropropyl)thieno[2,3-d]pyrimidine-2,4(1H,3H)-dione

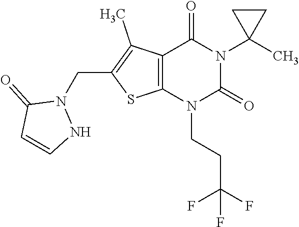 [SMILES: Cc1c(Cn2[nH]ccc2=O)sc2c1c(=O)n(C1(C)CC1)c(=O)n2CCC(F)(F)F]

Analogously to the method described in Ex. 200, 310 mg (0.539 mmol) of the compound from Ex. 455A were used to prepare 117 mg (50% of theory) of the title compound.

¹H-NMR (400 MHz, DMSO-d₆, δ/ppm): 11.23 (br. s, 1H), 7.15 (s, 1H), 5.30 (d, 1H), 5.14 (s, 2H), 4.18-3.88 (m, 2H), 2.80-2.63 (m, 2H), 2.46 (s, 3H), 1.32 (s, 3H), 1.01-0.68 (m, 4H).

LC/MS (Method 1, ESIneg): Rₜ=1.47 min, m/z=427.11 [M−H].

### Example 216

### 1-[(3,3-Difluorocyclobutyl)methyl]-5-methyl-6-[(2-oxo-2,3-dihydro-H-imidazol-1-yl)methyl]thieno[2,3-d]pyrimidine-2,4(1H,3H)-dione

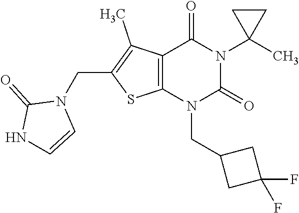 [SMILES: Cc1c(Cn2cc[nH]c2=O)sc2c1c(=O)n(C1(C)CC1)c(=O)n2CC1CC(F)(F)C1]

350 mg (0.699 mmol) of the compound from Ex. 394A were dissolved in 7 ml of methanol, and 1.4 ml of water and 1.4 ml of 1 M hydrochloric acid were added. After the reaction mixture had been stirred at RT for 2 days, it was diluted with water and extracted with ethyl acetate. The organic extract was washed successively with water and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 125 mg (40% of theory) of the title compound were obtained.

¹H-NMR (400 MHz, DMSO-d₆, δ/ppm): 9.98 (br. s, 1H), 6.41 (t, 1H), 6.34 (t, 1H), 4.78 (s, 2H), 4.18-3.81 (m, 2H), 2.75-2.61 (m, 2H), 2.45 (s, 3H), 1.32 (s, 3H), 1.02-0.67 (m, 4H).

LC/MS (Method 1, ESIpos): Rₜ=1.50 min, m/z=437.14 [M+H]⁺.

### Example 217

### 1-(2-Methoxyethyl)-5-methyl-3-(1-methylcyclopropyl)-6-[(2-oxo-2,3-dihydro-1H-imidazol-1-yl)methyl]thieno[2,3-d]pyrimidine-2,4(1H,3H)-dione

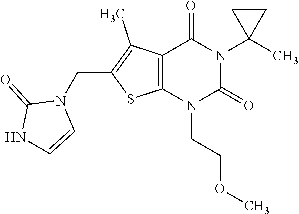 [SMILES: COCCn1c(=O)n(C2(C)CC2)c(=O)c2c(C)c(Cn3cc[nH]c3=O)sc21]

Analogously to the method described in Ex. 214, 305 mg (0.671 mmol) of the compound from Ex. 395A were used to prepare 70 mg (26% of theory) of the title compound.

¹H-NMR (400 MHz, DMSO-d₆, δ/ppm): 9.98 (br. s, 1H), 6.41 (t, 1H), 6.33 (t, 1H), 4.77 (s, 2H), 4.09-3.83 (m, 2H), 3.65-3.53 (m, 2H), 3.22 (s, 3H), 2.44 (s, 3H), 1.33 (s, 3H), 1.01-0.68 (m, 4H).

LC/MS (Method 1, ESIneg): Rₜ=1.17 min, m/z=389.13 [M−H].

### Example 218

### 5-Ethyl-3-(1-methylcyclopropyl)-6-[(2-oxoimidazolidin-1-yl)methyl]-1-(3,3,3-trifluoropropyl)thieno[2,3-d]pyrimidine-2,4(1H,3H)-dione

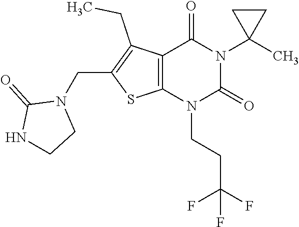 [SMILES: CCc1c(CN2CCNC2=O)sc2c1c(=O)n(C1(C)CC1)c(=O)n2CCC(F)(F)F]

Analogously to the method described in Ex. 17, 150 mg (0.323 mmol, 90% purity) of the compound from Ex. 469A were used to prepare 49 mg (34% of theory) of the title compound.

¹H-NMR (400 MHz, DMSO-d₆, δ/ppm): 6.54 (s, 1H), 4.36 (s, 2H), 4.18-3.92 (m, 2H), 3.29-3.16 (m, 4H), 2.94-2.69 (m, 4H), 1.34 (s, 3H), 1.08 (t, 3H), 0.97-0.77 (m, 4H).

LC/MS (Method 1, ESIpos): Rₜ=1.66 min, m/z=445.15 [M+H]⁺.

### Example 219

### 5-Ethyl-1-(2-methoxyethyl)-3-(1-methylcyclopropyl)-6-[(2-oxoimidazolidin-1-yl)methyl]thieno[2,3-d]pyrimidine-2,4(1H,3H)-dione

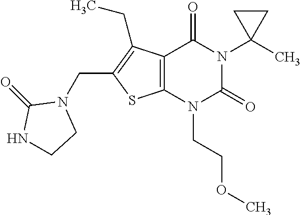 [SMILES: CCc1c(CN2CCNC2=O)sc2c1c(=O)n(C1(C)CC1)c(=O)n2CCOC]

Analogously to the method described in Ex. 17, 58 mg (0.107 mmol, 70% purity) of the compound from Ex. 470A were used to prepare 18 mg (42% of theory) of the title compound. The reaction time here was 2.5 days.

¹H-NMR (400 MHz, DMSO-d₆, δ/ppm): 6.52 (s, 1H), 4.34 (s, 2H), 4.14-3.81 (m, 2H), 3.67-3.56 (m, 2H), 3.24 (s, 3H), 2.94-2.75 (m, 2H), 1.34 (s, 3H), 1.08 (t, 3H), 0.97-0.77 (m, 4H).

LC/MS (Method 6, ESIpos): Rₜ=1.05 min, m/z=407 [M+H]⁺.

### Example 220

### 1,5-Dimethyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

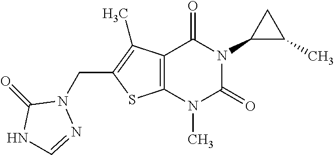 [SMILES: Cc1c(Cn2nc[nH]c2=O)sc2c1c(=O)n([C@H]1C[C@@H]1C)c(=O)n2C]

273 mg (0.499 mmol, 80% purity) of the compound from Ex. 473A were dissolved in a mixture of 11.5 ml each of methanol and trimethyl orthoformate, and 1.9 ml (7.49 mmol) of a 4 M solution of hydrogen chloride in dioxane were added at RT. After the reaction mixture had been stirred at RT for about 16 h, it was concentrated to dryness and the residue was subsequently purified by means of preparative HPLC (Method 13). After concentration of the product fraction and drying under high vacuum, 122 mg (70% of theory) of the title compound were obtained.

¹H-NMR (500 MHz, DMSO-d₆, δ/ppm): 11.59 (br. s, 1H), 7.82 (s, 1H), 4.92 (s, 2H), 3.35 (s, 3H), 2.44 (s, 3H), 2.25-2.18 (m, 1H), 1.14 (d, 3H), 1.03-0.92 (m, 1H), 0.86-0.77 (m, 2H).

LC/MS (Method 1, ESIpos): Rₜ=1.12 min, m/z=348.11 [M+H]⁺.

### Example 221

### 1,5-Dimethyl-3-(2-methylcyclopropyl)-6-[(2-oxoimidazolidin-1-yl)methyl]thieno[2,3-d]pyrimidine-2,4(1H,3H)-dione (trans racemate)

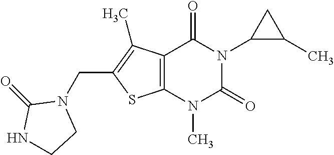 [SMILES: Cc1c(CN2CCNC2=O)sc2c1c(=O)n(C1CC1C)c(=O)n2C]

To a solution of 170 mg of the crude product from Ex. 363A (0.53 mmol, calculated with a theoretical purity of 100%) and 110 μl (0.79 mmol) of triethylamine in 3.4 ml of THF were added 103 mg (0.63 mmol) of 1,1′-carbonyldiimidazole (CDI), and the mixture was heated to 80° C. in a microwave apparatus (Biotage Initiator) for 10 min. The reaction mixture was then separated directly into its components by means of preparative HPLC (Method 17). After concentration of the product fractions and drying under high vacuum, 52 mg (28% of theory) of the title compound were obtained.

¹H-NMR (400 MHz, DMSO-d₆, δ/ppm): 6.51 (s, 1H), 4.34 (s, 2H), 3.36 (s, 3H), 3.22 (m, 4H), 2.38 (s, 3H), 2.22 (m, 1H), 1.15 (d, 3H), 1.02-0.93 (m, 1H), 0.86-0.79 (m, 2H).

LC/MS (Method 1, ESIpos): Rₜ=1.26 min, m/z=349.1 [M+H]⁺.

### Example 222

### 1,5-Dimethyl-3-(2-methylcyclopropyl)-6-[(2-oxoimidazolidin-1-yl)methyl]thieno[2,3-d]pyrimidine-2,4(1H,3H)-dione (trans enantiomer 1)

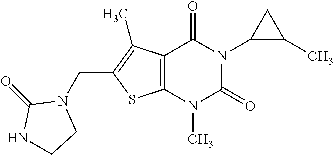 [SMILES: Cc1c(CN2CCNC2=O)sc2c1c(=O)n(C1CC1C)c(=O)n2C]

46 mg of the racemic compound from Ex. 221 were dissolved in 3 ml of ethanol and 1 ml of acetonitrile and separated into the enantiomers via preparative HPLC on a chiral phase [column: Daicel Chiracel OJ-H, 5 μm, 250 mm×20 mm; eluent: n-heptane/ethanol 1:1; flow rate: 20 ml/min; temperature: 40° C.; detection: 220 nm]. After concentration of the product fractions and drying of the solids under high vacuum, 19 mg (41% of theory) of enantiomer 1 were obtained (>99.0% ee, chiral analytical HPLC).

¹H-NMR (400 MHz, DMSO-d₆, δ/ppm): 6.51 (s, 1H), 4.34 (s, 2H), 3.37 (s, 3H), 3.22 (m, 4H), 2.38 (s, 3H), 2.22 (m, 1H), 1.15 (d, 3H), 1.02-0.93 (m, 1H), 0.86-0.79 (m, 2H).

Chiral analytical HPLC [column: Daicel IC-3, 3 μm 50 mm×4.6 mm; eluent: ethanol; flow rate: 1 ml/min; detection: 220 nm]: Rₜ=2.34 min.

### Example 223

### 1,5-Dimethyl-3-(2-methylcyclopropyl)-6-[(2-oxoimidazolidin-1-yl)methyl]thieno[2,3-d]pyrimidine-2,4(1H,3H)-dione (trans enantiomer 2)

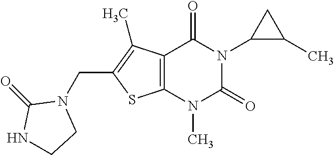 [SMILES: Cc1c(CN2CCNC2=O)sc2c1c(=O)n(C1CC1C)c(=O)n2C]

46 mg of the racemic compound from Ex. 221 were dissolved in 3 ml of ethanol and 1 ml of acetonitrile and separated into the enantiomers via preparative HPLC on a chiral phase [column: Daicel Chiracel OJ-H, 5 μm, 250 mm×20 mm; eluent: n-heptane/ethanol 1:1; flow rate: 20 ml/min; temperature: 40° C.; detection: 220 nm]. After concentration of the product fractions and drying of the solids under high vacuum, 21 mg (46% of theory) of enantiomer 2 were obtained (>99.0% ee, chiral analytical HPLC).

¹H-NMR (400 MHz, DMSO-d₆, δ/ppm): 6.51 (s, 1H), 4.34 (s, 2H), 3.37 (s, 3H), 3.22 (m, 4H), 2.38 (s, 3H), 2.22 (m, 1H), 1.15 (d, 3H), 1.02-0.93 (m, 1H), 0.86-0.79 (m, 2H).

Chiral analytical HPLC [column: Daicel IC-3, 3 μm 50 mm×4.6 mm; eluent: ethanol; flow rate: 1 ml/min; detection: 220 nm]: Rₜ=3.34 min.

### Example 224

### 1-Ethyl-5-methyl-3-(2-methylcyclopropyl)-6-[(2-oxoimidazolidin-1-yl)methyl]thieno[2,3-d]pyrimidine-2,4(1H,3H)-dione (trans racemate)

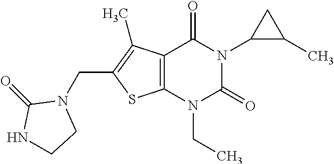 [SMILES: CCn1c(=O)n(C2CC2C)c(=O)c2c(C)c(CN3CCNC3=O)sc21]

To a solution of 238 mg of the crude product from Ex. 364A (0.71 mmol, calculated with a theoretical purity of 100% and a yield of 100%) and 148 μl (1.06 mmol) of triethylamine in 1.9 ml of THF were added 138 mg (0.85 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 18). After concentration of the product fractions and drying under high vacuum, 68 mg (27% of theory) of the title compound were obtained.

¹H-NMR (400 MHz, DMSO-d₆, δ/ppm): 6.51 (s, 1H), 4.34 (s, 2H), 3.85 (q, 2H), 3.23 (m, 4H), 2.38 (s, 3H), 2.23 (m, 1H), 1.22 (t, 3H), 1.15 (d, 3H), 1.02-0.93 (m, 1H), 0.86-0.79 (m, 2H).

LC/MS (Method 1, ESIpos): Rₜ=1.34 min, m/z=363.1 [M+H]⁺.

### Example 225

### 1-Ethyl-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

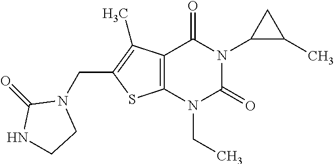 [SMILES: CCn1c(=O)n(C2CC2C)c(=O)c2c(C)c(CN3CCNC3=O)sc21]

60 mg of the racemic compound from Ex. 224 were dissolved in 2 ml of ethanol and 1 ml of acetonitrile and separated into the enantiomers via preparative HPLC on a chiral phase [column: Daicel Chiracel OX-H, 5 μm, 250 mm×20 mm; eluent: ethanol; flow rate: 15 ml/min; temperature: 50° C.; detection: 220 nm]. After concentration of the product fractions and drying of the solids under high vacuum, 24 mg (41% of theory) of enantiomer 1 were obtained (>99.0% ee, chiral analytical HPLC).

¹H-NMR (400 MHz, DMSO-d₆, δ/ppm): 6.51 (s, 1H), 4.34 (s, 2H), 3.85 (q, 2H), 3.23 (m, 4H), 2.38 (s, 3H), 2.23 (m, 1H), 1.22 (t, 3H), 1.15 (d, 3H), 1.02-0.93 (m, 1H), 0.86-0.79 (m, 2H). Chiral analytical HPLC [column: Daicel Chiralcel OX-3, 3 μm 50 mm×4.6 mm; eluent: ethanol; flow rate: 1 ml/min; temperature: 50° C.; detection: 220 nm]: Rₜ=3.05 min.

### Example 226

### 1-Ethyl-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

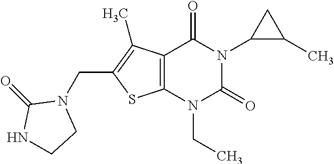 [SMILES: CCn1c(=O)n(C2CC2C)c(=O)c2c(C)c(CN3CCNC3=O)sc21]

60 mg of the racemic compound from Ex. 224 were dissolved in 2 ml of ethanol and 1 ml of acetonitrile and separated into the enantiomers via preparative HPLC on a chiral phase [column: Daicel Chiracel OX-H, 5 μm, 250 mm×20 mm; eluent: ethanol; flow rate: 15 ml/min; temperature: 50° C.; detection: 220 nm]. After concentration of the product fractions and drying of the solids under high vacuum, 26 mg (43% of theory) of enantiomer 2 were obtained (>99.0% ee, chiral analytical HPLC).

¹H-NMR (400 MHz, DMSO-d₆, δ/ppm): 6.51 (s, 1H), 4.34 (s, 2H), 3.85 (q, 2H), 3.23 (m, 4H), 2.38 (s, 3H), 2.23 (m, 1H), 1.22 (t, 3H), 1.15 (d, 3H), 1.02-0.93 (m, 1H), 0.86-0.79 (m, 2H).

Chiral analytical HPLC [column: Daicel Chiralcel OX-3, 3 μm 50 mm×4.6 mm; eluent: ethanol; flow rate: 1 ml/min; temperature: 50° C.; detection: 220 nm]: Rₜ=3.82 min.

### Example 227

### 5-Methyl-3-(2-methylcyclopropyl)-6-[(2-oxoimidazolidin-1-yl)methyl]-1-propylthieno[2,3-d]pyrimidine-2,4(1H,3H)-dione (trans racemate)

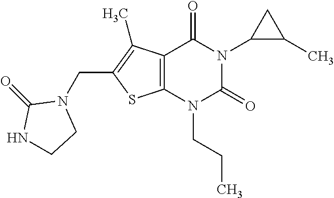 [SMILES: CCCn1c(=O)n(C2CC2C)c(=O)c2c(C)c(CN3CCNC3=O)sc21]

To a solution of 230 mg of the crude product from Ex. 365A (0.66 mmol, calculated with a theoretical purity of 100% and a yield of 100%) and 137 μl (0.98 mmol) of triethylamine in 4.2 ml of THF were added 128 mg (0.78 mmol) of 1,1′-carbonyldiimidazole (CDI), and the mixture was heated to 80° C. in a microwave apparatus (Biotage Initiator) for 5 min. The reaction mixture was then purified directly by means of preparative HPLC (Method 16). After concentration of the product fractions and drying under high vacuum, 154 mg (62% of theory) of the title compound were obtained.

¹H-NMR (400 MHz, DMSO-d₆, δ/ppm): 6.51 (broad, 1H), 4.34 (s, 2H), 3.78 (m, 2H), 3.22 (m, 4H), 2.37 (s, 3H), 2.23 (m, 1H), 1.67 (m, 2H), 1.15 (d, 3H), 1.02-0.93 (m, 1H), 0.90 (t, 3H), 0.86-0.79 (m, 2H).

LC/MS (Method 1, ESIpos): Rₜ=1.52 min, m/z=377.1 [M+H]⁺.

### Example 228

### 5-Methyl-3-(2-methylcyclopropyl)-6-[(2-oxoimidazolidin-1-yl)methyl]-1-propylthieno[2,3-d]pyrimidine-2,4(1H,3H)-dione (trans enantiomer 1)

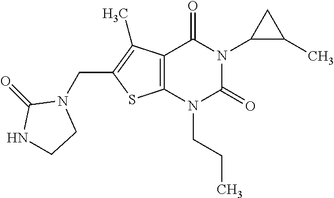 [SMILES: CCCn1c(=O)n(C2CC2C)c(=O)c2c(C)c(CN3CCNC3=O)sc21]

150 mg of the racemic compound from Ex. 227 were dissolved in 5 ml of ethanol/acetonitrile (1:1) and separated into the enantiomers via preparative HPLC on a chiral phase [column: Daicel Chiralpak OX-H 5 μm, 250 mm×20 mm; eluent: n-heptane/ethanol 1:1; flow rate: 40 ml/min; temperature: RT; detection: 220 nm]. After concentration of the product fractions and drying of the solids under high vacuum, 53 mg (35% of theory) of enantiomer 1 were obtained (>99.0% ee, chiral analytical HPLC).

¹H-NMR (400 MHz, DMSO-d₆, δ/ppm): 6.51 (s, 1H), 4.34 (s, 2H), 3.78 (m, 2H), 3.22 (m, 4H), 2.37 (s, 3H), 2.23 (m, 1H), 1.67 (m, 2H), 1.15 (d, 3H), 1.02-0.93 (m, 1H), 0.90 (t, 3H), 0.86-0.79 (m, 2H).

Chiral analytical HPLC [column: Daicel OX-3, 3 μm 50 mm×4.6 mm; eluent: ethanol; flow rate: 1 ml/min; detection: 220 nm]: Rₜ=2.94 min.

### Example 229

### 5-Methyl-3-(2-methylcyclopropyl)-6-[(2-oxoimidazolidin-1-yl)methyl]-1-propylthieno[2,3-d]pyrimidine-2,4(1H,3H)-dione (trans enantiomer 2)

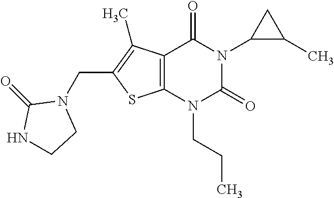 [SMILES: CCCn1c(=O)n(C2CC2C)c(=O)c2c(C)c(CN3CCNC3=O)sc21]

150 mg of the racemic compound from Ex. 227 were dissolved in 5 ml of ethanol/acetonitrile (1:1) and separated into the enantiomers via preparative HPLC on a chiral phase [column: Daicel Chiralpak OX-H 5 μm, 250 mm×20 mm; eluent: n-heptane/ethanol 1:1; flow rate: 40 ml/min; temperature: RT; detection: 220 nm]. After concentration of the product fractions and drying of the solids under high vacuum, 53 mg (35% of theory) of enantiomer 2 were obtained (>99.0% ee, chiral analytical HPLC).

¹H-NMR (400 MHz, DMSO-d₆, δ/ppm): 6.51 (s, 1H), 4.34 (s, 2H), 3.78 (m, 2H), 3.22 (m, 4H), 2.37 (s, 3H), 2.23 (m, 1H), 1.67 (m, 2H), 1.15 (d, 3H), 1.02-0.93 (m, 1H), 0.90 (t, 3H), 0.86-0.79 (m, 2H).

Chiral analytical HPLC [column: Daicel OX-3, 3 μm 50 mm×4.6 mm; eluent: ethanol; flow rate: 1 ml/min; detection: 220 nm]: Rₜ=4.51 min.

### Example 230

### 1-Butyl-5-methyl-3-[(1S,2S)-2-methylcyclopropyl]-6-[(2-oxoimidazolidin-1-yl)methyl]thieno[2,3-d]pyrimidine-2,4(1H,3H)-dione

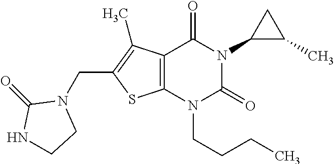 [SMILES: CCCCn1c(=O)n([C@H]2C[C@@H]2C)c(=O)c2c(C)c(CN3CCNC3=O)sc21]

To a solution of 95 mg of the crude product from Ex. 366A (0.26 mmol, calculated with a theoretical purity of 100%) and 54 μl (0.39 mmol) of triethylamine in 1.5 ml of THF were added 51 mg (0.31 mmol) of 1,1′-carbonyldiimidazole (CDI), and the mixture was heated to 80° C. in a microwave apparatus (Biotage Initiator) for 5 min. The reaction mixture was then purified directly by means of preparative HPLC (Method 16). After concentration of the product fractions and drying under high vacuum, 51 mg (50% of theory) of the title compound were obtained.

¹H-NMR (400 MHz, DMSO-d₆, δ/ppm): 6.52 (broad, 1H), 4.34 (s, 2H), 3.81 (m, 2H), 3.22 (m, 4H), 2.37 (s, 3H), 2.23 (m, 1H), 1.62 (m, 2H), 1.33 (m, 2H), 1.15 (d, 3H), 1.02-0.93 (m, 1H), 0.91 (t, 3H), 0.86-0.79 (m, 2H).

LC/MS (Method 1, ESIpos): Rₜ=1.63 min, m/z=391.1 [M+H]⁺.

### Example 231

### 1-(2-Fluoroethyl)-5-methyl-3-(2-methylcyclopropyl)-6-[(2-oxoimidazolidin-1-yl)methyl]thieno[2,3-d]pyrimidine-2,4(1H,3H)-dione (trans racemate)

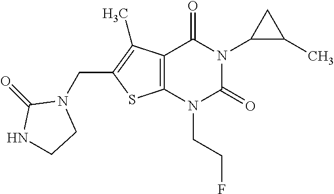 [SMILES: Cc1c(CN2CCNC2=O)sc2c1c(=O)n(C1CC1C)c(=O)n2CCF]

To a solution of 242 mg of the crude product from Ex. 367A (0.68 mmol, calculated with a theoretical purity of 100% and a yield of 100%) and 143 μl (1.02 mmol) of triethylamine in 2 ml of THF were added 133 mg (0.82 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 78 mg (30% of theory) of the title compound were obtained.

¹H-NMR (400 MHz, DMSO-d₆, δ/ppm): 6.51 (s, 1H), 4.70 (dt, 2H), 4.14 (dm, 2H), 3.22 (m, 4H), 2.37 (s, 3H), 2.24 (m, 1H), 1.15 (d, 3H), 1.05-0.94 (m, 1H), 0.88-0.80 (m, 2H).

LC/MS (Method 1, ESIpos): Rₜ=1.33 min, m/z=381.1 [M+H]⁺.

### Example 232

### 1-(2-Fluoroethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

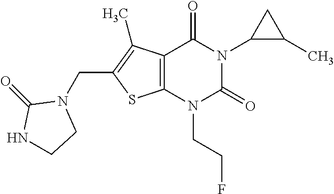 [SMILES: Cc1c(CN2CCNC2=O)sc2c1c(=O)n(C1CC1C)c(=O)n2CCF]

72 mg of the racemic compound from Ex. 231 were dissolved in a mixture of 2 ml of isopropanol, 1 ml of dichloromethane, 1 ml of acetonitrile and 2 ml of n-heptane, and, separated into the enantiomers via preparative HPLC on a chiral phase [column: Daicel Chiralpak ID 5 μm 250 mm×20 mm; eluent: n-heptane/isopropanol 1:1; flow rate: 15 ml/min; temperature: 40° C.; detection: 220 nm]. After concentration of the product fractions and drying of the solids under high vacuum, 33 mg (45% of theory) of enantiomer 1 were obtained (>99.0% ee, chiral analytical HPLC).

¹H-NMR (400 MHz, DMSO-d₆, δ/ppm): 6.51 (s, 1H), 4.70 (dt, 2H), 4.14 (dm, 2H), 3.22 (m, 4H), 2.37 (s, 3H), 2.24 (m, 1H), 1.15 (d, 3H), 1.05-0.94 (m, 1H), 0.88-0.80 (m, 2H).

Chiral analytical HPLC [column: Daicel ID-3, 3 μm 50 mm×4.6 mm; eluent: isopropanol; flow rate: 1 ml/min; detection: 220 nm]: Rₜ=3.55 min.

### Example 233

### 1-(2-Fluoroethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

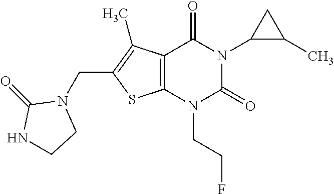 [SMILES: Cc1c(CN2CCNC2=O)sc2c1c(=O)n(C1CC1C)c(=O)n2CCF]

72 mg of the racemic compound from Ex. 231 were dissolved in a mixture of 2 ml of isopropanol, 1 ml of dichloromethane, 1 ml of acetonitrile and 2 ml of n-heptane, and, separated into the enantiomers via preparative HPLC on a chiral phase [column: Daicel Chiralpak ID 5 μm 250 mm×20 mm; eluent: n-heptane/isopropanol 1:1; flow rate: 15 ml/min; temperature: 40° C.; detection: 220 nm]. After concentration of the product fractions and drying of the solids under high vacuum, 28 mg (40% of theory) of enantiomer 2 were obtained (>99.0% ee, chiral analytical HPLC).

¹H-NMR (400 MHz, DMSO-d₆, δ/ppm): 6.51 (s, 1H), 4.70 (dt, 2H), 4.14 (dm, 2H), 3.22 (m, 4H), 2.37 (s, 3H), 2.24 (m, 1H), 1.15 (d, 3H), 1.05-0.94 (m, 1H), 0.88-0.80 (m, 2H).

Chiral analytical HPLC [column: Daicel ID-3, 3 μm 50 mm×4.6 mm; eluent: isopropanol; flow rate: 1 ml/min; detection: 220 nm]: 220 nm]: Rₜ=5.07 min.

### Example 234

### 1-(2,2-Difluoroethyl)-5-methyl-3-(2-methylcyclopropyl)-6-[(2-oxoimidazolidin-1-yl)methyl]thieno[2,3-d]pyrimidine-2,4(1H,3H)-dione (trans racemate)

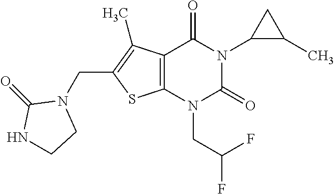 [SMILES: Cc1c(CN2CCNC2=O)sc2c1c(=O)n(C1CC1C)c(=O)n2CC(F)F]

To a solution of 189 mg of the crude product from Ex. 368A (0.51 mmol, calculated with a theoretical purity of 100% and a yield of 100%) and 106 μl (0.76 mmol) of triethylamine in 2 ml of THF were added 107 mg (0.66 mmol) of 1,1′-carbonyldiimidazole (CDI), and the mixture was heated to 80° C. in a microwave apparatus (Biotage Initiator) for 5 min. The reaction mixture was then purified directly by means of preparative HPLC (Method 16). After concentration of the product fractions and drying under high vacuum, 125 mg (59% of theory) of the title compound were obtained.

¹H-NMR (400 MHz, DMSO-d₆, δ/ppm): 6.52 (s, 1H), 6.31 (tt, 1H), 4.34 (s, 2H), 4.26 (tt, 2H), 3.22 (m, 4H), 2.38 (s, 3H), 2.25 (m, 1H), 1.15 (d, 3H), 1.06-0.97 (m, 1H), 0.88-0.80 (m, 2H).

LC/MS (Method 1, ESIpos): Rₜ=1.40 min, m/z=399.1 [M+H]⁺.

### Example 235

### 1-(2,2-Difluoroethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

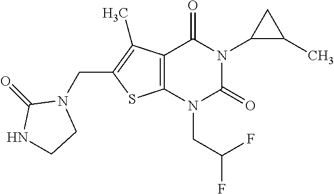 [SMILES: Cc1c(CN2CCNC2=O)sc2c1c(=O)n(C1CC1C)c(=O)n2CC(F)F]

115 mg of the racemic compound from Ex. 234 were dissolved in 5 ml of ethanol/acetonitrile (1:1) and separated into the enantiomers via preparative HPLC on a chiral phase [column: Daicel Chiralpak OX-H 5 μm, 250 mm×20 mm; eluent: n-heptane/ethanol 60:40; flow rate: 40 ml/min; temperature: 28° C.; detection: 220 nm]. After concentration of the product fractions and drying of the solids under high vacuum, 31 mg (27% of theory) of enantiomer 1 were obtained (>99.0% ee, chiral analytical HPLC).

¹H-NMR (400 MHz, DMSO-d₆, δ/ppm): 6.52 (s, 1H), 6.31 (tt, 1H), 4.34 (s, 2H), 4.26 (tt, 2H), 3.22 (m, 4H), 2.38 (s, 3H), 2.25 (m, 1H), 1.15 (d, 3H), 1.06-0.97 (m, 1H), 0.88-0.80 (m, 2H).

Chiral analytical HPLC [column: Daicel OX-3, 3 μm 50 mm×4.6 mm; eluent: ethanol; flow rate: 1 ml/min; detection: 220 nm]: Rₜ=3.25 min.

### Example 236

### 1-(2,2-Difluoroethyl)-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

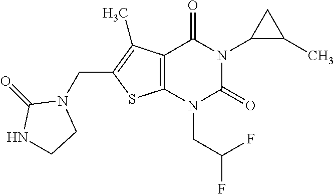 [SMILES: Cc1c(CN2CCNC2=O)sc2c1c(=O)n(C1CC1C)c(=O)n2CC(F)F]

115 mg of the racemic compound from Ex. 234 were dissolved in 5 ml of ethanol/acetonitrile (1:1) and separated into the enantiomers via preparative HPLC on a chiral phase [column: Daicel Chiralpak OX-H 5 μm, 250 mm×20 mm; eluent: n-heptane/ethanol 60:40; flow rate: 40 ml/min; temperature: 28° C.; detection: 220 nm]. After concentration of the product fractions and drying of the solids under high vacuum, 32 mg (28% of theory) of enantiomer 2 were obtained (>99.0% ee, chiral analytical HPLC).

¹H-NMR (400 MHz, DMSO-d₆, δ/ppm): 6.52 (s, 1H), 6.31 (tt, 1H), 4.34 (s, 2H), 4.26 (tt, 2H), 3.22 (m, 4H), 2.38 (s, 3H), 2.25 (m, 1H), 1.15 (d, 3H), 1.06-0.97 (m, 1H), 0.88-0.80 (m, 2H).

Chiral analytical HPLC [column: Daicel OX-3, 3 μm 50 mm×4.6 mm; eluent: ethanol; flow rate: 1 ml/min; detection: 220 nm]: Rₜ=4.28 min.

### Example 237

### 5-Methyl-3-(2-methylcyclopropyl)-6-[(2-oxoimidazolidin-1-yl)methyl]-1-(2,2,2-trifluoroethyl)thieno[2,3-d]pyrimidine-2,4(1H,3H)-dione (trans racemate)

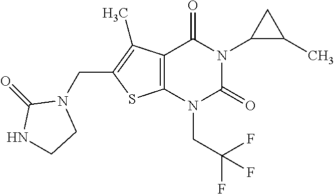 [SMILES: Cc1c(CN2CCNC2=O)sc2c1c(=O)n(C1CC1C)c(=O)n2CC(F)(F)F]

To a solution of 109 mg of the crude product from Ex. 369A (0.28 mmol, calculated with a theoretical purity of 100% and a yield of 100%) and 58 μl (0.42 mmol) of triethylamine in 0.75 ml of THF were added 54 mg (0.33 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 39 mg (34% of theory) of the title compound were obtained.

¹H-NMR (400 MHz, DMSO-d₆, δ/ppm): 6.54 (broad, 1H), 4.77 (m, 2H), 4.35 (s, 2H), 3.22 (m, 4H), 2.38 (s, 3H), 2.27 (m, 1H), 1.15 (d, 3H), 1.04-0.97 (m, 1H), 0.87-0.81 (m, 2H).

LC/MS (Method 1, ESIpos): Rₜ=1.58 min, m/z=417.1 [M+H]⁺.

### Example 238

### 5-Methyl-3-(2-methylcyclopropyl)-6-[(2-oxoimidazolidin-1-yl)methyl]-1-(2,2,2-trifluoroethyl)thieno[2,3-d]pyrimidine-2,4(1H,3H)-dione (trans enantiomer 1)

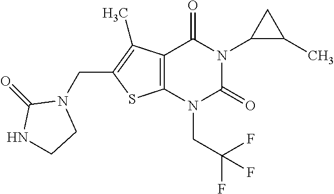 [SMILES: Cc1c(CN2CCNC2=O)sc2c1c(=O)n(C1CC1C)c(=O)n2CC(F)(F)F]

33 mg of the racemic compound from Ex. 237 were dissolved in 3 ml of isopropanol/acetonitrile (40:60) and separated into the enantiomers via preparative HPLC on a chiral phase [column: Daicel Chiralcel OX-H, 5 μm, 250 mm×20 mm; eluent: n-heptane/isopropanol 30:70; flow rate: 35 ml/min; temperature: 28° C.; detection: 220 nm]. After concentration of the product fractions and drying of the solids under high vacuum, 11 mg (32% of theory) of enantiomer 1 were obtained (>99.0% ee, chiral analytical HPLC).

¹H-NMR (400 MHz, DMSO-d₆, δ/ppm): 6.53 (s, 1H), 4.77 (m, 2H), 4.35 (s, 2H), 3.22 (m, 4H), 2.38 (s, 3H), 2.27 (m, 1H), 1.15 (d, 3H), 1.04-0.97 (m, 1H), 0.87-0.81 (m, 2H).

Chiral analytical HPLC [column: Daicel Chiralpak OX-3, 3 μm 50 mm×4.6 mm; eluent: n-heptane/isopropanol 1:1; flow rate: 1 ml/min; detection: 220 nm]: Rₜ=2.59 min.

### Example 239

### 5-Methyl-3-(2-methylcyclopropyl)-6-[(2-oxoimidazolidin-1-yl)methyl]-1-(2,2,2-trifluoroethyl)thieno[2,3-d]pyrimidine-2,4(1H,3H)-dione (trans enantiomer 2)

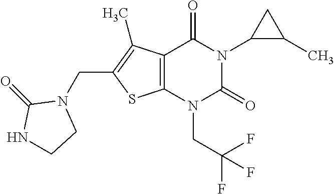 [SMILES: Cc1c(CN2CCNC2=O)sc2c1c(=O)n(C1CC1C)c(=O)n2CC(F)(F)F]

33 mg of the racemic compound from Ex. 237 were dissolved in 3 ml of isopropanol/acetonitrile (40:60) and separated into the enantiomers via preparative HPLC on a chiral phase [column: Daicel Chiralcel OX-H, 5 μm, 250 mm×20 mm; eluent: n-heptane/isopropanol 30:70; flow rate: 35 ml/min; temperature: 28° C.; detection: 220 nm]. After concentration of the product fractions and drying of the solids under high vacuum, 11 mg (32% of theory) of enantiomer 2 were obtained (>99.0% ee, chiral analytical HPLC).

¹H-NMR (400 MHz, DMSO-d₆, δ/ppm): 6.53 (s, 1H), 4.77 (m, 2H), 4.35 (s, 2H), 3.22 (m, 4H), 2.38 (s, 3H), 2.27 (m, 1H), 1.15 (d, 3H), 1.04-0.97 (m, 1H), 0.87-0.81 (m, 2H).

Chiral analytical HPLC [column: Daicel Chiralpak OX-3, 3 μm 50 mm×4.6 mm; eluent: n-heptane/isopropanol 1:1; flow rate: 1 ml/min; detection: 220 nm]: Rₜ=4.61 min.

### Example 240

### 5-Methyl-3-(2-methylcyclopropyl)-6-[(2-oxoimidazolidin-1-yl)methyl]-1-(3,3,3-trifluoropropyl)thieno[2,3-d]pyrimidine-2,4(1H,3H)-dione (trans enantiomer 1)

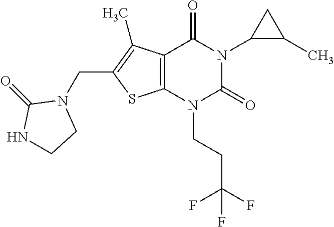 [SMILES: Cc1c(CN2CCNC2=O)sc2c1c(=O)n(C1CC1C)c(=O)n2CCC(F)(F)F]

225 mg of the racemic compound from Ex. 93 were separated into the enantiomers by preparative SFC-HPLC on a chiral phase [instrument: Sepiatec Prep SFC100; column: Reprosil chiral NR, 8 μm, 250 mm×30 mm; eluent A: carbon dioxide, eluent B: ethanol, isocratic with 29% B; flow rate: 100 ml/min; temperature: 40° C.; BPR: 150 bar; detection: MWD 220 nm]. The respective product fractions were concentrated on a rotary evaporator, admixed with acetonitrile/water (1:1) and freeze-dried. 110 mg (44% of theory) of the title compound (enantiomer 1) and 110 mg (44% of theory) of enantiomer 2 (see Ex. 241) were obtained.

¹H-NMR (400 MHz, DMSO-d₆, δ/ppm): 6.55 (s, 1H), 4.35 (s, 2H), 4.04 (q, 2H), 3.17-3.28 (m, 4H), 2.66-2.80 (m, 2H), 2.38 (s, 3H), 2.20-2.26 (m, 1H), 1.15 (d, 3H), 0.94-1.04 (m, 1H), 0.79-0.86 (m, 2H).

Chiral analytical SFC-HPLC [instrument: Agilent 1260, Aurora SFC module; column: Reprosil chiral NR, 5 μm, 100 mm×4.6 mm; eluent A: carbon dioxide, eluent B: ethanol, isocratic with 29% B; flow rate: 4 ml/min; temperature: 37.5° C.; BPR: 100 bar; detection: MWD 220 nm]: Rₜ=3.07 min.

### Example 241

### 5-Methyl-3-(2-methylcyclopropyl)-6-[(2-oxoimidazolidin-1-yl)methyl]-1-(3,3,3-trifluoropropyl)thieno[2,3-d]pyrimidine-2,4(1H,3H)-dione (trans enantiomer 2)

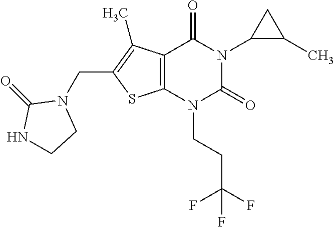 [SMILES: Cc1c(CN2CCNC2=O)sc2c1c(=O)n(C1CC1C)c(=O)n2CCC(F)(F)F]

The title compound (110 mg) was obtained as the second enantiomer from the preparative HPLC separation (see Ex. 240) of the racemate from Ex. 93.

¹H-NMR (400 MHz, DMSO-d₆, δ/ppm): 6.55 (s, 1H), 4.35 (s, 2H), 3.97-4.12 (m, 2H), 3.17-3.27 (m, 4H), 2.66-2.79 (m, 2H), 2.38 (s, 3H), 2.21-2.26 (m, 1H), 1.15 (d, 3H), 0.93-1.03 (m, 1H), 0.79-0.87 (m, 2H).

Chiral analytical SFC-HPLC [instrument: Agilent 1260, Aurora SFC module; column: Reprosil chiral NR, 5 μm, 100 mm×4.6 mm; eluent A: carbon dioxide, eluent B: ethanol, isocratic with 29% B; flow rate: 4 ml/min; temperature: 37.5° C.; BPR: 100 bar; detection: MWD 220 nm]: Rₜ=4.24 min.

### Example 242

### 5-Methyl-3-(2-methylcyclopropyl)-6-[(2-oxoimidazolidin-1-yl)methyl]-1-(4,4,4-trifluorobutyl)thieno[2,3-d]pyrimidine-2,4(1H,3H)-dione (trans racemate)

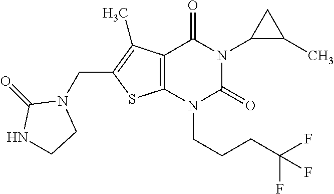 [SMILES: Cc1c(CN2CCNC2=O)sc2c1c(=O)n(C1CC1C)c(=O)n2CCCC(F)(F)F]

To a solution of 257 mg of the crude product from Ex. 370A (0.61 mmol, calculated with a theoretical purity of 100% and a yield of 100%) and 128 μl (0.92 mmol) of triethylamine in 1.9 ml of THF were added 119 mg (0.74 mmol) of 1,1′-carbonyldiimidazole (CDI), and the mixture was heated to 80° C. in a microwave apparatus (Biotage Initiator) for 5 min. The reaction mixture was then purified directly by means of preparative HPLC (Method 16). After concentration of the product fractions and drying under high vacuum, 167 mg (58% of theory) of the title compound were obtained.

¹H-NMR (400 MHz, DMSO-d₆, δ/ppm): 6.51 (s, 1H), 4.34 (s, 2H), 3.91 (m, 2H), 3.22 (m, 4H), 2.41 (m, 2H), 2.38 (s, 3H), 2.22 (m, 1H), 1.87 (m, 2H), 1.15 (d, 3H), 1.04-0.97 (m, 1H), 0.87-0.78 (m, 2H).

LC/MS (Method 1, ESIpos): Rₜ=1.65 min, m/z=445.1 [M+H]⁺.

### Example 243

### 5-Methyl-3-(2-methylcyclopropyl)-6-[(2-oxoimidazolidin-1-yl)methyl]-1-(4,4,4-trifluorobutyl)thieno[2,3-d]pyrimidine-2,4(1H,3H)-dione (trans enantiomer 1)

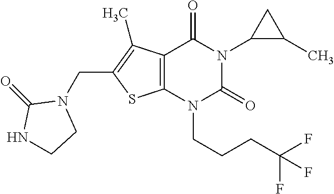 [SMILES: Cc1c(CN2CCNC2=O)sc2c1c(=O)n(C1CC1C)c(=O)n2CCCC(F)(F)F]

150 mg of the racemic compound from Ex. 242 were separated into the enantiomers via preparative HPLC on a chiral phase [column: Daicel Chiralcel OX-H, 5 μm, 250 mm×20 mm; eluent: n-heptane/ethanol 35:65; flow rate: 15 ml/min; temperature: 25° C.; detection: 220 nm]. After concentration of the product fractions and drying of the solids under high vacuum, 58 mg (39% of theory) of enantiomer 1 were obtained (>99.0% ee, chiral analytical HPLC).

¹H-NMR (400 MHz, DMSO-d₆, δ/ppm): 6.51 (s, 1H), 4.34 (s, 2H), 3.91 (m, 2H), 3.22 (m, 4H), 2.41 (m, 2H), 2.38 (s, 3H), 2.22 (m, 1H), 1.87 (m, 2H), 1.15 (d, 3H), 1.04-0.97 (m, 1H), 0.87-0.78 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=2.12 min.

### Example 244

### 5-Methyl-3-(2-methylcyclopropyl)-6-[(2-oxoimidazolidin-1-yl)methyl]-1-(4,4,4-trifluorobutyl)thieno[2,3-d]pyrimidine-2,4(1H,3H)-dione (trans enantiomer 2)

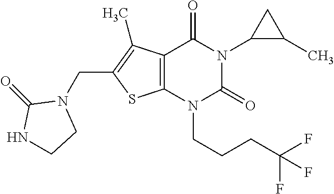 [SMILES: Cc1c(CN2CCNC2=O)sc2c1c(=O)n(C1CC1C)c(=O)n2CCCC(F)(F)F]

150 mg of the racemic compound from Ex. 242 were separated into the enantiomers via preparative HPLC on a chiral phase [column: Daicel Chiralcel OX-H, 5 μm, 250 mm×20 mm; eluent: n-heptane/ethanol 35:65; flow rate: 15 ml/min; temperature: 25° C.; detection: 220 nm]. After concentration of the product fractions and drying of the solids under high vacuum, 60 mg (40% of theory) of enantiomer 2 were obtained (>99.0% ee, chiral analytical HPLC).

¹H-NMR (400 MHz, DMSO-d₆, δ/ppm): 6.51 (s, 1H), 4.34 (s, 2H), 3.91 (m, 2H), 3.22 (m, 4H), 2.41 (m, 2H), 2.38 (s, 3H), 2.22 (m, 1H), 1.87 (m, 2H), 1.15 (d, 3H), 1.04-0.97 (m, 1H), 0.87-0.78 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=2.88 min.

### Example 245

### 5-Methyl-3-(2-methylcyclopropyl)-6-[(2-oxoimidazolidin-1-yl)methyl]-1-(3,3,4,4-tetrafluorobutyl)thieno[2,3-d]pyrimidine-2,4(1H,3H)-dione (trans racemate)

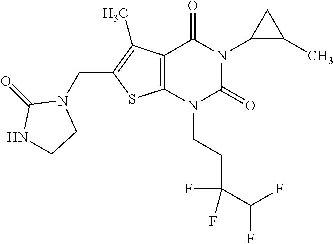 [SMILES: Cc1c(CN2CCNC2=O)sc2c1c(=O)n(C1CC1C)c(=O)n2CCC(F)(F)C(F)F]

To a solution of 245 mg (0.48 mmol, 86% purity) of the crude product from Ex. 371A and 100 μl (0.72 mmol) of triethylamine in 2.8 ml of THF were added 93 mg (0.57 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 127 mg (57% of theory) of the title compound were obtained.

¹H-NMR (400 MHz, DMSO-d₆, δ/ppm): 6.54 (tt, 1H), 6.52 (s, 1H), 4.35 (s, 2H), 4.04 (m, 2H), 3.22 (m, 4H), 2.38 (s, 3H), 2.24 (m, 1H), 1.15 (d, 3H), 1.03-0.94 (m, 1H), 0.86-0.79 (m, 2H).

LC/MS (Method 2, ESIpos): Rₜ=0.87 min, m/z=463.0 [M+H]⁺.

### Example 246

### 5-Methyl-3-(2-methylcyclopropyl)-6-[(2-oxoimidazolidin-1-yl)methyl]-1-(3,3,4,4-tetrafluorobutyl)thieno[2,3-d]pyrimidine-2,4(1H,3H)-dione (trans enantiomer 1)

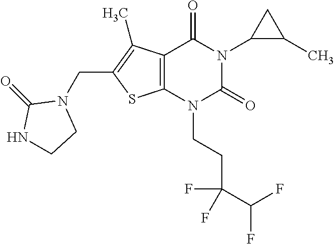 [SMILES: Cc1c(CN2CCNC2=O)sc2c1c(=O)n(C1CC1C)c(=O)n2CCC(F)(F)C(F)F]

120 mg of the racemic compound from Ex. 245 were dissolved in about 20 ml of ethanol/acetonitrile (9:1) and separated into the enantiomers via preparative HPLC on a chiral phase [column: Daicel Chiralpak OX-H 5 μm, 250 mm×30 mm; eluent: n-heptane/ethanol 60:40; flow rate: 60 ml/min; temperature: 25° C.; detection: 220 nm]. After concentration of the product fractions and drying of the solids under high vacuum, 46 mg (39% of theory) of enantiomer 1 were obtained (>99.0% ee, chiral analytical HPLC).

¹H-NMR (400 MHz, DMSO-d₆, δ/ppm): 6.54 (tt, 1H), 6.52 (s, 1H), 4.35 (s, 2H), 4.05 (m, 2H), 3.22 (m, 4H), 2.38 (s, 3H), 2.22 (m, 1H), 1.15 (d, 3H), 1.03-0.94 (m, 1H), 0.87-0.80 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=2.27 min.

### Example 247

### 5-Methyl-3-(2-methylcyclopropyl)-6-[(2-oxoimidazolidin-1-yl)methyl]-1-(3,3,4,4-tetrafluorobutyl)thieno[2,3-d]pyrimidine-2,4(1H,3H)-dione (trans enantiomer 2)

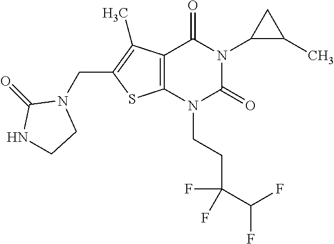 [SMILES: Cc1c(CN2CCNC2=O)sc2c1c(=O)n(C1CC1C)c(=O)n2CCC(F)(F)C(F)F]

120 mg of the racemic compound from Ex. 245 were dissolved in about 20 ml of ethanol/acetonitrile (9:1) and separated into the enantiomers via preparative HPLC on a chiral phase [column: Daicel Chiralpak OX-H 5 μm, 250 mm×30 mm; eluent: n-heptane/ethanol 60:40; flow rate: 60 ml/min; temperature: 25° C.; detection: 220 nm]. After concentration of the product fractions and drying of the solids under high vacuum, 43 mg (36% of theory) of enantiomer 2 were obtained (94.4% ee, chiral analytical HPLC).

¹H-NMR (400 MHz, DMSO-d₆, δ/ppm): 6.54 (tt, 1H), 6.52 (s, 1H), 4.35 (s, 2H), 4.05 (m, 2H), 3.22 (m, 4H), 2.38 (s, 3H), 2.22 (m, 1H), 1.15 (d, 3H), 1.03-0.94 (m, 1H), 0.87-0.80 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=3.32 min.

### Example 248

### 1-(Cyclobutylmethyl)-5-methyl-3-[(1S,2S)-2-methylcyclopropyl]-6-[(2-oxoimidazolidin-1-yl)methyl]thieno[2,3-d]pyrimidine-2,4(1H,3H)-dione

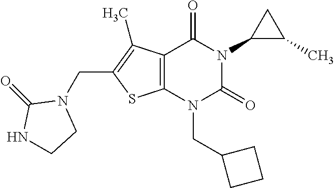 [SMILES: Cc1c(CN2CCNC2=O)sc2c1c(=O)n([C@H]1C[C@@H]1C)c(=O)n2CC1CCC1]

To a solution of 120 mg of the crude product from Ex. 372A (0.32 mmol, calculated with a theoretical purity of 100% and a yield of 100%) and 67 μl (0.48 mmol) of triethylamine in 2 ml of THF were added 62 mg (0.38 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 65 mg (50% of theory) of the title compound were obtained.

¹H-NMR (400 MHz, DMSO-d₆, δ/ppm): 6.51 (s, 1H), 4.33 (s, 2H), 3.21 (m, 4H), 2.74 (m, 1H), 2.37 (s, 3H), 2.22 (m, 1H), 1.97 (m, 2H), 1.91 (m, 4H), 1.15 (d, 3H), 1.01-0.92 (m, 1H), 0.85-0.78 (m, 2H).

LC/MS (Method 1, ESIpos): Rₜ=1.72 min, m/z=403.1 [M+H]⁺.

### Example 249

### 1-[(2,2-Difluorocyclopropyl)methyl]-5-methyl-3-[(1S,2S)-2-methylcyclopropyl]-6-[(2-oxoimidazolidin-1-yl)methyl]thieno[2,3-d]pyrimidine-2,4(1H,3H)-dione (diastereomer mixture)

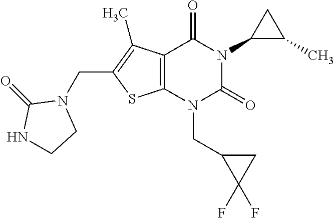 [SMILES: Cc1c(CN2CCNC2=O)sc2c1c(=O)n([C@H]1C[C@@H]1C)c(=O)n2CC1CC1(F)F]

To a solution of 120 mg of the crude product from Ex. 373A (0.30 mmol, calculated with a theoretical purity of 100% and a yield of 100%) and 63 μl (0.45 mmol) of triethylamine in 1.9 ml of THF were added 59 mg (0.36 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 64 mg (50% of theory) of the title compound were obtained.

¹H-NMR (400 MHz, DMSO-d₆, δ/ppm): 6.52 (s, 1H), 4.35 (s, 2H), 4.06 (m, 1H), 3.90 (m, 1H), 3.22 (m, 4H), 2.38 (s, 3H), 2.24 (m, 1H), 2.18 (m, 1H), 1.69 (m, 1H), 1.44 (m, 1H), 1.15 (d, 3H), 1.03-0.97 (m, 1H), 0.87-0.80 (m, 2H).

LC/MS (Method 2, ESIpos): Rₜ=1.58 min, m/z=425.1 [M+H]⁺.

### Example 250

### 1-[(2,2-Difluorocyclopropyl)methyl]-5-methyl-3-[(1S,2S)-2-methylcyclopropyl]-6-[(2-oxoimidazolidin-1-yl)methyl]thieno[2,3-d]pyrimidine-2,4(1H,3H)-dione (diastereomer 1)

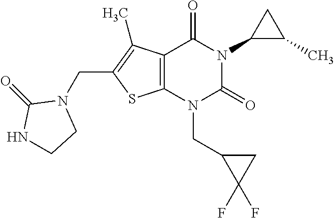 [SMILES: Cc1c(CN2CCNC2=O)sc2c1c(=O)n([C@H]1C[C@@H]1C)c(=O)n2CC1CC1(F)F]

58 mg of the diastereomer mixture from Ex. 249 were dissolved in 5 ml of ethanol/acetonitrile (6:4) and separated into the enantiomerically pure diastereomers via preparative HPLC on a chiral phase [column: Daicel Chiralpak OJ-H, 5 μm, 250 mm×20 mm; eluent: n-heptane/ethanol 80:20; flow rate: 35 ml/min; temperature: 28° C.; detection: 220 nm]. After concentration of the product fractions and drying of the solids under high vacuum, 25 mg (44% of theory) of diastereomer 1 were obtained (>99.0% de, chiral analytical HPLC).

¹H-NMR (400 MHz, DMSO-d₆, δ/ppm): 6.52 (s, 1H), 4.35 (s, 2H), 4.06 (m, 1H), 3.91 (m, 1H), 3.22 (m, 4H), 2.38 (s, 3H), 2.24 (m, 1H), 2.18 (m, 1H), 1.69 (m, 1H), 1.44 (m, 1H), 1.15 (d, 3H), 1.03-0.97 (m, 1H), 0.87-0.80 (m, 2H).

Chiral analytical HPLC [column: Daicel Chiralpak IA-3, 3 μm 50 mm×4.6 mm; eluent: isohexane/ethanol 1:1; flow rate: 1 ml/min; detection: 220 nm]: Rₜ=1.41 min.

### Example 251

### 1-[(2,2-Difluorocyclopropyl)methyl]-5-methyl-3-[(1S,2S)-2-methylcyclopropyl]-6-[(2-oxoimidazolidin-1-yl)methyl]thieno[2,3-d]pyrimidine-2,4(1H,3H)-dione (diastereomer 2)

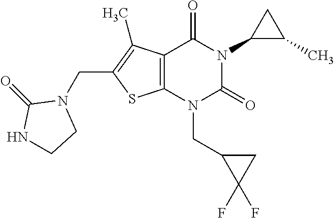 [SMILES: Cc1c(CN2CCNC2=O)sc2c1c(=O)n([C@H]1C[C@@H]1C)c(=O)n2CC1CC1(F)F]

58 mg of the diastereomer mixture from Ex. 249 were dissolved in 5 ml of ethanol/acetonitrile (6:4) and separated into the enantiomerically pure diastereomers via preparative HPLC on a chiral phase [column: Daicel Chiralpak OJ-H, 5 μm, 250 mm×20 mm; eluent: n-heptane/ethanol 80:20; flow rate: 35 ml/min; temperature: 28° C.; detection: 220 nm]. After concentration of the product fractions and drying of the solids under high vacuum, 25 mg (44% of theory) of diastereomer 2 were obtained (>99.0% de, chiral analytical HPLC).

¹H-NMR (400 MHz, DMSO-d₆, δ/ppm): 6.52 (s, 1H), 4.35 (s, 2H), 4.06 (m, 1H), 3.91 (m, 1H), 3.22 (m, 4H), 2.38 (s, 3H), 2.24 (m, 1H), 2.18 (m, 1H), 1.69 (m, 1H), 1.44 (m, 1H), 1.15 (d, 3H), 1.03-0.97 (m, 1H), 0.87-0.80 (m, 2H).

Chiral analytical HPLC [column: Daicel Chiralpak IA-3, 3 μm 50 mm×4.6 mm; eluent: isohexane/ethanol 1:1; flow rate: 1 ml/min; detection: 220 nm]: Rₜ=1.53 min.

### Example 252

### 1-[(3,3-Difluorocyclobutyl)methyl]-5-methyl-3-(2-methylcyclopropyl)-6-[(2-oxoimidazolidin-1-yl)methyl]thieno[2,3-d]pyrimidine-2,4(1H,3H)-dione (trans racemate)

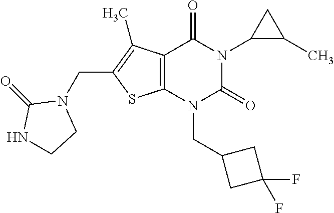 [SMILES: Cc1c(CN2CCNC2=O)sc2c1c(=O)n(C1CC1C)c(=O)n2CC1CC(F)(F)C1]

To a solution of 190 mg of the crude product from Ex. 374A (0.46 mmol, calculated with a theoretical purity of 100% and a yield of 100%) and 96 μl (0.69 mmol) of triethylamine in 1.4 ml of THF were added 89 mg (0.55 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 74 mg (37% of theory) of the title compound were obtained.

¹H-NMR (400 MHz, DMSO-d₆, δ/ppm): 6.52 (s, 1H), 4.34 (s, 2H), 4.01 (m, 2H), 3.22 (m, 4H), 2.73-2.43 (m, 5H), 2.37 (s, 3H), 2.23 (m, 1H), 1.15 (d, 3H), 1.02-0.95 (m, 1H), 0.85-0.78 (m, 2H).

LC/MS (Method 1, ESIpos): Rₜ=1.65 min, m/z=439.1 [M+H]⁺.

### Example 253

### 1-[(3,3-Difluorocyclobutyl)methyl]-5-methyl-3-(2-methylcyclopropyl)-6-[(2-oxoimidazolidin-1-yl)methyl]thieno[2,3-d]pyrimidine-2,4(1H,3H)-dione (trans enantiomer 1)

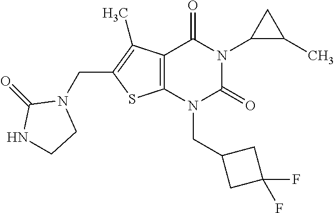 [SMILES: Cc1c(CN2CCNC2=O)sc2c1c(=O)n(C1CC1C)c(=O)n2CC1CC(F)(F)C1]

70 mg of the racemic compound from Ex. 252 were dissolved in 4 ml of ethanol/acetonitrile (1:1) and separated into the enantiomers via preparative HPLC on a chiral phase [column: Daicel Chiralpak OJ-H, 5 μm, 250 mm×20 mm; eluent: n-heptane/ethanol 70:30; flow rate: 20 ml/min; temperature: 28° C.; detection: 220 nm]. After concentration of the product fractions and drying of the solids under high vacuum, 29 mg (41% of theory) of enantiomer 1 were obtained (>99.0% ee, chiral analytical HPLC).

¹H-NMR (400 MHz, DMSO-d₆, δ/ppm): 6.52 (s, 1H), 4.34 (s, 2H), 4.01 (m, 2H), 3.22 (m, 4H), 2.73-2.43 (m, 5H), 2.37 (s, 3H), 2.23 (m, 1H), 1.15 (d, 3H), 1.02-0.95 (m, 1H), 0.85-0.78 (m, 2H).

Chiral analytical HPLC [column: Daicel Chiralpak IC-3, 3 μm 50 mm×4.6 mm; eluent: ethanol; flow rate: 1 ml/min; detection: 220 nm]: Rₜ=1.55 min.

### Example 254

### 1-[(3,3-Difluorocyclobutyl)methyl]-5-methyl-3-(2-methylcyclopropyl)-6-[(2-oxoimidazolidin-1-yl)methyl]thieno[2,3-d]pyrimidine-2,4(1H,3H)-dione (trans enantiomer 2)

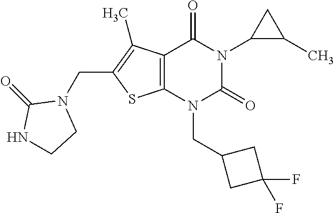 [SMILES: Cc1c(CN2CCNC2=O)sc2c1c(=O)n(C1CC1C)c(=O)n2CC1CC(F)(F)C1]

70 mg of the racemic compound from Ex. 252 were dissolved in 4 ml of ethanol/acetonitrile (1:1) and separated into the enantiomers via preparative HPLC on a chiral phase [column: Daicel Chiralpak OJ-H, 5 μm, 250 mm×20 mm; eluent: n-heptane/ethanol 70:30; flow rate: 20 ml/min; temperature: 28° C.; detection: 220 nm]. After concentration of the product fractions and drying of the solids under high vacuum, 28 mg (39% of theory) of enantiomer 2 were obtained (97.3% ee, chiral analytical HPLC).

¹H-NMR (400 MHz, DMSO-d₆, δ/ppm): 6.52 (s, 1H), 4.34 (s, 2H), 4.01 (m, 2H), 3.22 (m, 4H), 2.73-2.43 (m, 5H), 2.37 (s, 3H), 2.23 (m, 1H), 1.15 (d, 3H), 1.02-0.95 (m, 1H), 0.85-0.78 (m, 2H).

Chiral analytical HPLC [column: Daicel Chiralpak IC-3, 3 μm 50 mm×4.6 mm; eluent: ethanol; flow rate: 1 ml/min; detection: 220 nm]: Rₜ=1.93 min.

### Example 255

### 1-[(3,3-Difluorocyclopenty)methy]-5-methyethyl-3-[(S,2S)-2-methylcyclopropyl]-6-[(2-oxoimidazolidin-1-yl)methyl]thieno[2,3-d]pyrimidine-2,4(1H,3H)-dione (diastereomer mixture)

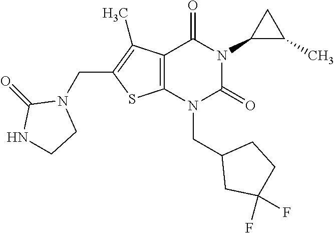 [SMILES: Cc1c(CN2CCNC2=O)sc2c1c(=O)n([C@H]1C[C@@H]1C)c(=O)n2CC1CCC(F)(F)C1]

To a solution of 102 mg of the crude product from Ex. 375A (0.24 mmol, calculated with a theoretical purity of 100% and a yield of 100%) and 50 μl (0.34 mmol) of triethylamine in 2 ml of THF were added 47 mg (0.28 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 35 mg (32% of theory) of the title compound were obtained.

¹H-NMR (400 MHz, DMSO-d₆, δ/ppm): 6.51 (s, 1H), 4.34 (s, 2H), 3.88 (m, 2H), 3.22 (m, 4H), 2.61 (m, 1H), 2.38 (s, 3H), 2.34-1.82 (m, 6H), 1.58 (m, 1H), 1.15 (d, 3H), 1.03-0.94 (m, 1H), 0.86-0.79 (m, 2H).

LC/MS (Method 1, ESIpos): Rₜ=1.67 min, m/z=453.1 [M+H]⁺.

### Example 256

### 1-[(3,3-Difluorocyclopentyl)methyl]-5-methyl-3-[(1S,2S)-2-methylcyclopropyl]-6-[(2-oxoimidazolidin-1-yl)methyl]thieno[2,3-d]pyrimidine-2,4(1H,3H)-dione (diastereomer 1)

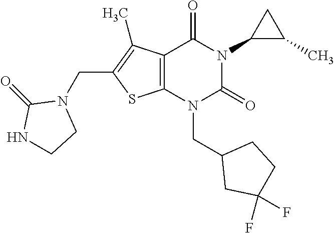 [SMILES: Cc1c(CN2CCNC2=O)sc2c1c(=O)n([C@H]1C[C@@H]1C)c(=O)n2CC1CCC(F)(F)C1]

33 mg of the diastereomer mixture from Ex. 255 were dissolved in 4 ml of ethanol/acetonitrile (1:1) and separated into the enantiomerically pure diastereomers via preparative HPLC on a chiral phase [column: Daicel Chiralcel OX-H, 5 μm, 250 mm×20 mm; eluent: n-heptane/ethanol 60:40; flow rate: 20 ml/min; temperature: 35° C.; detection: 220 nm]. After concentration of the product fractions and drying of the solids under high vacuum, 16 mg (49% of theory) of diastereomer 1 were obtained (>99% de, chiral analytical HPLC).

¹H-NMR (400 MHz, DMSO-d₆, δ/ppm): 6.51 (s, 1H), 4.34 (s, 2H), 3.88 (m, 2H), 3.22 (m, 4H), 2.61 (m, 1H), 2.38 (s, 3H), 2.34-1.82 (m, 6H), 1.58 (m, 1H), 1.15 (d, 3H), 1.03-0.94 (m, 1H), 0.86-0.79 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=3.59 min.

### Example 257

### 1-[(3,3-Difluorocyclopentyl)methyl]-5-methyl-3-[(1S,2S)-2-methylcyclopropyl]-6-[(2-oxoimidazolidin-1-yl)methyl]thieno[2,3-d]pyrimidine-2,4(1H,3H)-dione (diastereomer 2)

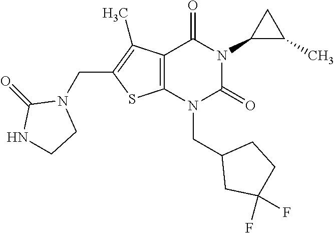 [SMILES: Cc1c(CN2CCNC2=O)sc2c1c(=O)n([C@H]1C[C@@H]1C)c(=O)n2CC1CCC(F)(F)C1]

33 mg of the diastereomer mixture from Ex. 255 were dissolved in 4 ml of ethanol/acetonitrile (1:1) and separated into the enantiomerically pure diastereomers via preparative HPLC on a chiral phase [column: Daicel Chiralcel OX-H, 5 μm, 250 mm×20 mm; eluent: n-heptane/ethanol 60:40; flow rate: 20 ml/min; temperature: 35° C.; detection: 220 nm]. After concentration of the product fractions and drying of the solids under high vacuum, 17 mg (50% of theory) of diastereomer 2 were obtained (94.7% de, chiral analytical HPLC).

¹H-NMR (400 MHz, DMSO-d₆, δ/ppm): 6.51 (s, 1H), 4.34 (s, 2H), 3.88 (m, 2H), 3.22 (m, 4H), 2.61 (m, 1H), 2.38 (s, 3H), 2.34-1.82 (m, 6H), 1.58 (m, 1H), 1.15 (d, 3H), 1.03-0.94 (m, 1H), 0.86-0.79 (m, 2H).

Chiral analytical HPLC [column: Daicel Chiralpak OX-3, 3 μm 50 mm×4.6 mm; eluent: ethanol; flow rate: 1 ml/min; detection: 220 nm]: Rₜ=4.33 min.

### Example 258

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (cis racemate)

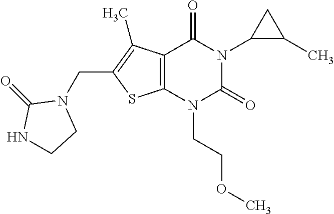 [SMILES: COCCn1c(=O)n(C2CC2C)c(=O)c2c(C)c(CN3CCNC3=O)sc21]

600 mg (1.47 mmol, 90% purity) of the compound from Ex. 376A were dissolved in 25 ml of THF, and 308 μl (2.21 mmol) of triethylamine and 287 mg (1.77 mmol) of CDI were added. The mixture was stirred at RT for about 16 h. It was then concentrated to dryness on a rotary evaporator. The remaining residue was purified by means of preparative HPLC (Method 11). Combination of the product fractions and freeze-drying gave 373 mg (64% of theory) of the title compound.

¹H-NMR (400 MHz, DMSO-d₆, δ/ppm): 6.51 (s, 1H), 4.33 (s, 2H), 4.13-3.85 (m, 2H), 3.67-3.55 (m, 2H), 3.29-3.16 (m, 4H), 3.22 (s, 3H), 2.62 (td, 1H), 2.37 (s, 3H), 1.27-1.18 (m, 2H), 0.84 (d, 3H), 0.60-0.45 (m, 1H).

LC/MS (Method 1, ESIpos): Rₜ=1.23 min, m/z=393.16 [M+H]⁺.

### Example 259

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (cis enantiomer 1)

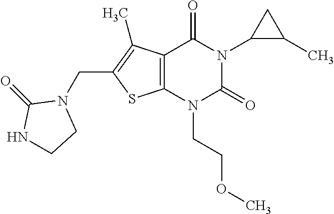 [SMILES: COCCn1c(=O)n(C2CC2C)c(=O)c2c(C)c(CN3CCNC3=O)sc21]

360 mg of the racemic compound from Ex. 258 were dissolved in 25 ml of methanol/ethanol/acetonitrile (2:2:1) and, in 26 portions, separated into the enantiomers by preparative SFC-HPLC on a chiral phase [column: Daicel Chiralpak OJ-H, 5 μm, 250 mm×20 mm; eluent: carbon dioxide/methanol 86:14; flow rate: 85 ml/min; temperature: 40° C.; detection: 210 nm]. After concentration of the product fractions and drying of the solids under high vacuum, 146 mg (81% of theory) of enantiomer 1 were obtained (>99% ee, chiral analytical HPLC).

¹H-NMR (400 MHz, DMSO-d₆, δ/ppm): 6.51 (s, 1H), 4.33 (s, 2H), 4.12-3.86 (m, 2H), 3.67-3.55 (m, 2H), 3.29-3.16 (m, 4H), 3.23 (s, 3H), 2.62 (td, 1H), 2.37 (s, 3H), 1.28-1.17 (m, 2H), 0.84 (d, 3H), 0.57-0.47 (m, 1H).

Chiral analytical SFC-HPLC [column: Daicel Chiralpak OJ-H, 5 μm, 50 mm×4.6 mm; eluent: carbon dioxide/methanol 90:10; flow rate: 3 ml/min; temperature: 40° C.; detection: 210 nm]: Rₜ=2.24 min.

### Example 260

### 1-(2-Methoxyethyl)-5-methyl-3-(2-methylcyclopropyl)-6-[(2-oxoimidazolidin-1-yl)methyl]thieno[2,3-d]pyrimidine-2,4(1H,3H)-dione (cis enantiomer 2)

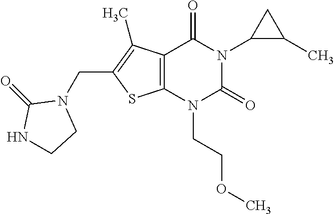 [SMILES: COCCn1c(=O)n(C2CC2C)c(=O)c2c(C)c(CN3CCNC3=O)sc21]

75 mg (41% of theory) of enantiomer 2 were obtained from the preparative HPLC separation on a chiral phase described in Ex. 259 (>99% ee, chiral analytical HPLC).

¹H-NMR (400 MHz, DMSO-d₆, δ/ppm): 6.51 (s, 1H), 4.33 (s, 2H), 4.13-3.86 (m, 2H), 3.67-3.55 (m, 2H), 3.29-3.17 (m, 4H), 3.22 (s, 3H), 2.62 (td, 1H), 2.37 (s, 3H), 1.29-1.16 (m, 2H), 0.84 (d, 3H), 0.58-0.47 (m, 1H).

Chiral analytical SFC-HPLC [column: Daicel Chiralpak OJ-H, 5 μm, 50 mm×4.6 mm; eluent: carbon dioxide/methanol 90:10; flow rate: 3 ml/min; temperature: 40° C.; detection: 210 nm]: Rₜ=2.87 min.

### Example 261

### 5-Methyl-3-(2-methylcyclopropyl)-6-[(2-oxoimidazolidin-1-yl)methyl]-1-[2-(trifluoromethoxy)ethyl]thieno[2,3-d]pyrimidine-2,4(1H,3H)-dione (trans racemate)

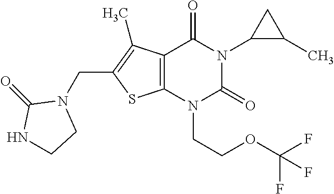 [SMILES: Cc1c(CN2CCNC2=O)sc2c1c(=O)n(C1CC1C)c(=O)n2CCOC(F)(F)F]

193 mg (0.438 mmol, 95% purity) of the compound from Ex. 378A were dissolved in 5 ml of THF, and 92 μl (0.657 mmol) of triethylamine and 85 mg (0.525 mmol) of CDI were added. The mixture was stirred at RT for about 16 h. It was then concentrated to dryness on a rotary evaporator. The remaining residue was purified by means of preparative HPLC (Method 11). Combination of the product fractions and freeze-drying gave 125 mg (63% of theory) of the title compound.

¹H-NMR (400 MHz, DMSO-d₆, δ/ppm): 6.52 (s, 1H), 4.37 (t, 2H), 4.34 (s, 2H), 4.21-4.08 (m, 2H), 3.27-3.15 (m, 4H), 2.38 (s, 3H), 2.28-2.22 (m, 1H), 1.15 (d, 3H), 1.04-0.93 (m, 1H), 0.87-0.78 (m, 2H).

LC/MS (Method 1, ESIpos): Rₜ=1.59 min, m/z=447.13 [M+H]⁺.

### Example 262

### 5-Methyl-3-(2-methylcyclopropyl)-6-[(2-oxoimidazolidin-1-yl)methyl]-1-[2-(trifluoromethoxy)ethyl]thieno[2,3-d]pyrimidine-2,4(1H,3H)-dione (trans enantiomer 1)

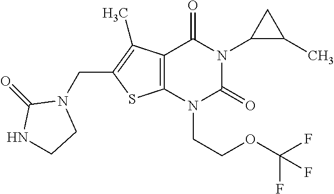 [SMILES: Cc1c(CN2CCNC2=O)sc2c1c(=O)n(C1CC1C)c(=O)n2CCOC(F)(F)F]

120 mg of the racemic compound from Ex. 261 were dissolved in a mixture of 4 ml of ethanol and 2 ml of acetonitrile and, in 12 portions, separated into the enantiomers via preparative HPLC on a chiral phase [column: Daicel Chiralcel OX-H, 5 μm, 250 mm×20 mm; eluent: isohexane/ethanol 60:40; flow rate: 20 ml/min; temperature: 50° C.; detection: 220 nm]. After concentration of the product fractions and drying of the solids under high vacuum, 53 mg (88% of theory) of enantiomer 1 were obtained (>99% ee, chiral analytical HPLC).

¹H-NMR (400 MHz, DMSO-d₆, δ/ppm): 6.52 (s, 1H), 4.37 (t, 2H), 4.34 (s, 2H), 4.21-4.07 (m, 2H), 3.27-3.15 (m, 4H), 2.38 (s, 3H), 2.25 (td, 1H), 1.15 (d, 3H), 1.05-0.92 (m, 1H), 0.86-0.79 (m, 2H).

Chiral analytical HPLC [column: Daicel Chiralpak OX-3, 3 μm 50 mm×4.6 mm; eluent: isohexane/ethanol 1:1; flow rate: 1 ml/min; temperature: 40° C.; detection: 220 nm]: Rₜ=2.42 min.

### Example 263

### 5-Methyl-3-(2-methylcyclopropyl)-6-[(2-oxoimidazolidin-1-yl)methyl]-1-[2-(trifluoromethoxy)ethyl]thieno[2,3-d]pyrimidine-2,4(1H,3H)-dione (trans enantiomer 2)

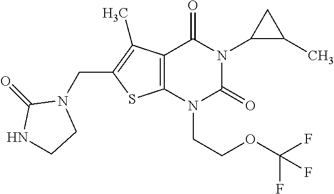 [SMILES: Cc1c(CN2CCNC2=O)sc2c1c(=O)n(C1CC1C)c(=O)n2CCOC(F)(F)F]

65 mg of enantiomer 2 were obtained from the preparative HPLC separation on a chiral phase described in Ex. 262 (>99% ee, chiral analytical HPLC).

¹H-NMR (400 MHz, DMSO-d₆, δ/ppm): 6.52 (s, 1H), 4.37 (t, 2H), 4.34 (s, 2H), 4.21-4.07 (m, 2H), 3.26-3.16 (m, 4H), 2.38 (s, 3H), 2.25 (td, 1H), 1.15 (d, 3H), 1.04-0.92 (m, 1H), 0.87-0.79 (m, 2H).

Chiral analytical HPLC [column: Daicel Chiralpak OX-3, 3 μm 50 mm×4.6 mm; eluent: isohexane/ethanol 1:1; flow rate: 1 ml/min; temperature: 40° C.; detection: 220 nm]: Rₜ=3.84 min.

### Example 264

### [1-({5-Methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]cyanamide (trans racemate)

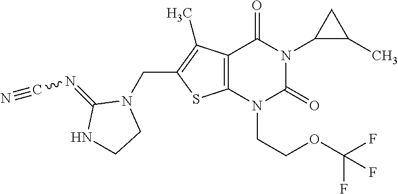 [SMILES: Cc1c(CN2CCNC2=NC#N)sc2c1c(=O)n(C1CC1C)c(=O)n2CCOC(F)(F)F]

390 mg (0.881 mmol, 95% purity) of the compound from Ex. 378A were dissolved in 10 ml of DMF, and 193 mg (1.32 mmol) of dimethyl N-cyanodithioiminocarbonate and 244 mg (1.76 mmol) of potassium carbonate were added. The mixture was stirred at 80° C. in a microwave oven (Biotage Initiator with dynamic control of irradiation power) for 4 h. Water was then added to the reaction mixture which was extracted with tert-butyl methyl ether. The organic extract was washed with saturated sodium chloride solution, dried over anhydrous magnesium sulfate, filtered and concentrated. The crude product was purified by preparative HPLC (Method 11). The product fractions were combined, concentrated by evaporation and dried under high vacuum. 114 mg (26% of theory, 96% purity) of the title compound were obtained.

¹H-NMR (400 MHz, DMSO-d₆, δ/ppm): 8.06 (s, 1H), 4.47 (s, 2H), 4.37 (t, 2H), 4.22-4.09 (m, 2H), 3.48-3.36 (m, 4H), 2.39 (s, 3H), 2.29-2.21 (m, 1H), 1.15 (d, 3H), 1.04-0.92 (m, 1H), 0.88-0.77 (m, 2H).

LC/MS (Method 1, ESIpos): Rₜ=1.68 min, m/z=471.14 [M+H]⁺.

### Example 265

### [1-({5-Methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]cyanamide (trans enantiomer 1)

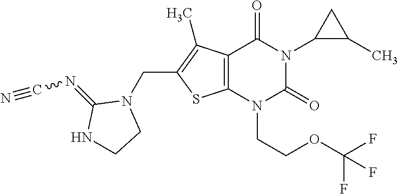 [SMILES: Cc1c(CN2CCNC2=NC#N)sc2c1c(=O)n(C1CC1C)c(=O)n2CCOC(F)(F)F]

111 mg of the racemic compound from Ex. 264 were dissolved in a mixture of 3 ml of dichloromethane and 2 ml of ethanol and, in 16 portions, separated into the enantiomers via preparative HPLC on a chiral phase [column: Daicel Chiralcel OX-H, 5 μm, 250 mm×20 mm; eluent: ethanol; flow rate: 15 ml/min; temperature: 25° C.; detection: 210 nm]. After concentration of the product fractions and drying of the solids under high vacuum, 44 mg (79% of theory) of enantiomer 1 were obtained (>99% ee, chiral analytical HPLC).

¹H-NMR (400 MHz, DMSO-d₆, δ/ppm): 8.06 (s, 1H), 4.47 (s, 2H), 4.37 (t, 2H), 4.22-4.09 (m, 2H), 3.48-3.35 (m, 4H), 2.39 (s, 3H), 2.29-2.21 (m, 1H), 1.15 (d, 3H), 1.04-0.92 (m, 1H), 0.88-0.78 (m, 2H).

Chiral analytical HPLC [column: Daicel Chiralpak OX-3, 3 μm 50 mm×4.6 mm; eluent: ethanol; flow rate: 1 ml/min; temperature: 25° C.; detection: 220 nm]: Rₜ=1.37 min.

### Example 266

### [1-({5-Methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]cyanamide (trans enantiomer 2)

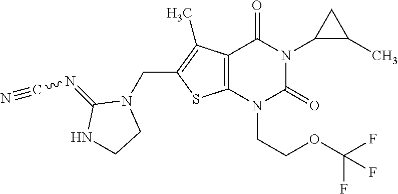 [SMILES: Cc1c(CN2CCNC2=NC#N)sc2c1c(=O)n(C1CC1C)c(=O)n2CCOC(F)(F)F]

43 mg (75% of theory, 98% purity) of enantiomer 2 were obtained from the preparative HPLC separation on a chiral phase described in Ex. 265 (99% ee, chiral analytical HPLC).

¹H-NMR (400 MHz, DMSO-d₆, δ/ppm): 8.06 (s, 1H), 4.47 (s, 2H), 4.37 (t, 2H), 4.22-4.09 (m, 2H), 3.48-3.36 (m, 4H), 2.39 (s, 3H), 2.29-2.21 (m, 1H), 1.15 (d, 3H), 1.04-0.92 (m, 1H), 0.88-0.77 (m, 2H).

Chiral analytical HPLC [column: Daicel Chiralpak OX-3, 3 μm 50 mm×4.6 mm; eluent: ethanol; flow rate: 1 ml/min; temperature: 25° C.; detection: 220 nm]: Rₜ=2.25 min.

### Example 267

### Methyl [1-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]carbamate (trans racemate)

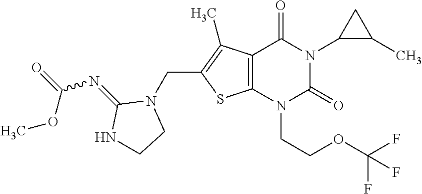

260 mg (0.557 mmol, 90% purity) of the compound from Ex. 378A and 155 μl (1.11 mmol) of triethylamine were dissolved in 10 ml of dichloromethane, and a solution of 174 mg (1.11 mmol) of methyl (dichloromethylene)carbamate in 5 ml of dichloromethane was added dropwise. After the reaction mixture had been stirred at RT for 3 h, it was concentrated to dryness. The remaining residue was purified by means of preparative HPLC (Method 11). Concentration of the product fraction and drying of the residue under high vacuum gave 205 mg (73% of theory) of the title compound.

¹H-NMR (400 MHz, DMSO-d₆, δ/ppm): 8.29 (broad, 1H), 4.62 (s, 2H), 4.37 (t, 2H), 4.21-4.06 (m, 2H), 3.60 (s, 3H), 3.54-3.45 (m, 2H), 3.41-3.33 (m, 2H, partially concealed by water signal), 2.40 (s, 3H), 2.29-2.21 (m, 1H), 1.15 (d, 3H), 1.05-0.91 (m, 1H), 0.88-0.78 (m, 2H).

LC/MS (Method 1, ESIpos): Rₜ=1.62 min, m/z=504.15 [M+H]⁺.

### Example 268

### Methyl [1-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]carbamate (trans enantiomer 1)

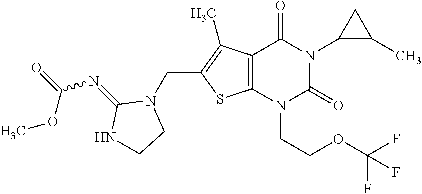

195 mg of the racemic compound from Ex. 267 were dissolved in 20 ml of a methanol/acetonitrile mixture and, in 7 portions, separated into the enantiomers by preparative SFC-HPLC on a chiral phase [column: Daicel Chiralcel OX-H, 5 μm, 250 mm×20 mm; eluent: carbon dioxide/ethanol 3:1; flow rate: 80 ml/min; temperature: 40° C.; detection: 210 nm]. After concentration of the product fractions and drying of the solids under high vacuum, 66 mg (67% of theory) of enantiomer 1 were obtained (>99% ee, chiral analytical HPLC).

¹H-NMR (400 MHz, DMSO-d₆, δ/ppm): 8.02 (s, 1H), 4.56 (s, 2H), 4.36 (t, 2H), 4.19-4.06 (m, 2H), 3.53 (s, 3H), 3.48-3.40 (m, 2H), 3.35-3.29 (m, 2H, substantially concealed by water signal), 2.40 (s, 3H), 2.29-2.21 (m, 1H), 1.15 (d, 3H), 1.04-0.92 (m, 1H), 0.87-0.78 (m, 2H).

Chiral analytical SFC-HPLC [column: Daicel Chiralpak OX-H 5 μm, 50 mm×4.6 mm; eluent: carbon dioxide/ethanol 60:40; flow rate: 3 ml/min; temperature: 40° C.; detection: 210 nm]: Rₜ=1.67 min.

### Example 269

### Methyl [1-({5-methyl-3-(2-methylcyclopropyl)-2,4-dioxo-1-[2-(trifluoromethoxy)ethyl]-1,2,3,4-tetrahydrothieno[2,3-d]pyrimidin-6-yl}methyl)imidazolidin-2-ylidene]carbamate (trans enantiomer 2)

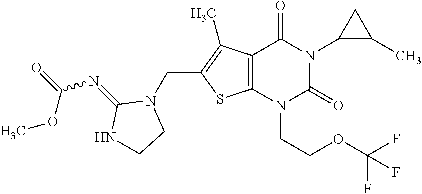

48 mg (49% of theory) of enantiomer 2 were obtained from the preparative HPLC separation on a chiral phase described in Ex. 268 (>99% ee, chiral analytical HPLC).

¹H-NMR (400 MHz, DMSO-d₆, δ/ppm): 8.08 (1H), 4.57 (2H), 4.36 (2H), 4.13 (2H), 3.54 (3H), 3.46 (2H), 2.40 (3H), 2.25 (1H), 1.15 (3H), 0.98 (1H), 0.83 (2H).

Chiral analytical SFC-HPLC [column: Daicel Chiralpak OX-H 5 μm, 50 mm×4.6 mm; eluent: carbon dioxide/ethanol 60:40; flow rate: 3 ml/min; temperature: 40° C.; detection: 210 nm]: Rₜ=4.10 min.

### Example 270

### 5-Methyl-3-(2-methylcyclopropyl)-6-[(2-oxo-2,3-dihydro-1H-imidazol-1-yl)methyl]-1-[2-(trifluoromethoxy)ethyl]thieno[2,3-d]pyrimidine-2,4(1H,3H)-dione (trans racemate)

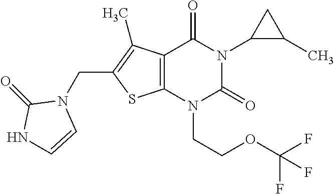 [SMILES: Cc1c(Cn2cc[nH]c2=O)sc2c1c(=O)n(C1CC1C)c(=O)n2CCOC(F)(F)F]

540 mg (0.881 mmol, 83% purity) of the compound from Ex. 396A were dissolved in 10 ml of methanol, and 1.8 ml of water and 1.8 ml of 1 M hydrochloric acid were added. After the reaction mixture had been stirred at RT for 2.5 days, it was diluted with water and extracted with ethyl acetate. The organic extract was washed successively with water and saturated sodium chloride solution. After drying over anhydrous magnesium sulfate, the mixture was filtered and concentrated. The solid residue was purified by means of preparative HPLC (Method 11). After concentration of the product fractions and drying under high vacuum, 242 mg (61% of theory) of the title compound were obtained.

¹H-NMR (400 MHz, DMSO-d₆, δ/ppm): 9.98 (br. s, 1H), 6.40 (t, 1H), 6.33 (t, 1H), 4.78 (s, 2H), 4.36 (t, 2H), 4.19-4.05 (m, 2H), 2.44 (s, 3H), 2.28-2.21 (m, 1H), 1.15 (d, 3H), 1.03-0.91 (m, 1H), 0.87-0.77 (m, 2H).

LC/MS (Method 1, ESIpos): Rₜ=1.51 min, m/z=445.11 [M+H]⁺.

### Example 271

### 5-Methyl-3-(2-methylcyclopropyl)-6-[(2-oxo-2,3-dihydro-1H-imidazol-1-yl)methyl]-1-[2-(trifluoromethoxy)ethyl]thieno[2,3-d]pyrimidine-2,4(1H,3H)-dione (trans enantiomer 1)

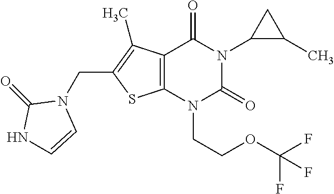 [SMILES: Cc1c(Cn2cc[nH]c2=O)sc2c1c(=O)n(C1CC1C)c(=O)n2CCOC(F)(F)F]

233 mg of the racemic compound from Ex. 270 were dissolved in 5 ml of acetonitrile/ethanol (1:1) and, in 62 portions, separated into the enantiomers by preparative HPLC on a chiral phase [column: Daicel Chiralcel OX-H, 5 μm, 250 mm×20 mm; eluent: n-heptane/ethanol 1:1; flow rate: 40 ml/min; temperature: 28° C.; detection: 220 nm]. After concentration of the product fractions and drying of the solids under high vacuum, 93 mg (79% of theory) of enantiomer 1 were obtained (99% ee, chiral analytical HPLC).

¹H-NMR (400 MHz, DMSO-d₆, δ/ppm): 9.98 (br. s, 1H), 6.40 (dd, 1H), 6.33 (t, 1H), 4.78 (s, 2H), 4.39-4.32 (m, 2H), 4.19-4.05 (m, 2H), 2.44 (s, 3H), 2.28-2.21 (m, 1H), 1.15 (d, 3H), 0.98 (tq, 1H), 0.87-0.77 (m, 2H).

Chiral analytical HPLC [column: Daicel Chiralpak OX-3, 3 μm 50 mm×4.6 mm; eluent: n-heptane/ethanol 1:1; flow rate: 1 ml/min; temperature: 25° C.; detection: 220 nm]: Rₜ=1.64 min.

### Example 272

### 5-Methyl-3-(2-methylcyclopropyl)-6-[(2-oxo-2,3-dihydro-1H-imidazol-1-yl)methyl]-1-[2-(trifluoromethoxy)ethyl]thieno[2,3-d]pyrimidine-2,4(1H,3H)-dione (trans enantiomer 2)

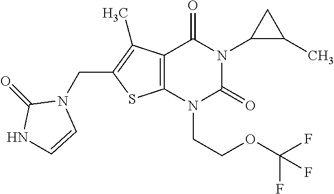 [SMILES: Cc1c(Cn2cc[nH]c2=O)sc2c1c(=O)n(C1CC1C)c(=O)n2CCOC(F)(F)F]

95 mg (81% of theory) of enantiomer 2 were obtained from the preparative HPLC separation on a chiral phase described in Ex. 271 (99% ee, chiral analytical HPLC).

¹H-NMR (400 MHz, DMSO-d₆, δ/ppm): 9.98 (br. s, 1H), 6.45-6.37 (m, 1H), 6.33 (t, 1H), 4.78 (s, 2H), 4.36 (t, 2H), 4.20-4.04 (m, 2H), 2.44 (s, 3H), 2.28-2.21 (m, 1H), 1.15 (d, 3H), 1.03-0.91 (m, 1H), 0.87-0.78 (m, 2H).

Chiral analytical HPLC [column: Daicel Chiralpak OX-3, 3 μm 50 mm×4.6 mm; eluent: n-heptane/ethanol 1:1; flow rate: 1 ml/min; temperature: 25° C.; detection: 220 nm]: Rₜ=2.23 min.

### Example 273

### 5-Methyl-3-(2-methylcyclopropyl)-6-[(5-oxo-2,5-dihydro-1H-pyrazol-1-yl)methyl]-1-[2-(trifluoromethoxy)ethyl]thieno[2,3-d]pyrimidine-2,4(1H,3H)-dione (trans racemate)

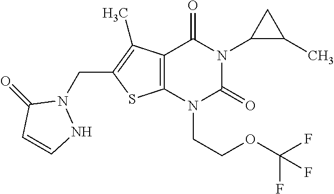 [SMILES: Cc1c(Cn2[nH]ccc2=O)sc2c1c(=O)n(C1CC1C)c(=O)n2CCOC(F)(F)F]

Analogously to the method described in Ex. 200, 300 mg (0.508 mmol) of the compound from Ex. 456A were used to prepare 158 mg (70% of theory) of the title compound.

¹H-NMR (400 MHz, DMSO-d₆, δ/ppm): 11.12 (br. s, 1H), 7.14 (br. s, 1H), 5.30 (s, 1H), 5.13 (s, 2H), 4.34 (t, 2H), 4.19-4.04 (m, 2H), 2.45 (s, 3H), 2.28-2.20 (m, 1H), 1.14 (d, 3H), 1.03-0.91 (m, 1H), 0.87-0.77 (m, 2H).

LC/MS (Method 1, ESIneg): Rₜ=1.57 min, m/z=443.10 [M−H].

### Example 274

### 5-Methyl-3-(2-methylcyclopropyl)-6-[(5-oxo-2,5-dihydro-1H-pyrazol-1-yl)methyl]-1-[2-(trifluoromethoxy)ethyl]thieno[2,3-d]pyrimidine-2,4(1H,3H)-dione (trans enantiomer 1)

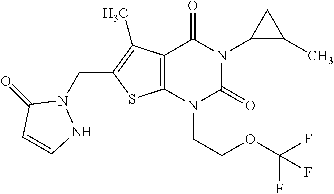 [SMILES: Cc1c(Cn2[nH]ccc2=O)sc2c1c(=O)n(C1CC1C)c(=O)n2CCOC(F)(F)F]

149 mg of the racemic compound from Ex. 273 were dissolved in a mixture of 6 ml of ethanol and 2 ml of acetonitrile and, in 16 portions, separated into the enantiomers via preparative HPLC on a chiral phase [column: Daicel Chiralcel OX-H, 5 μm, 250 mm×20 mm; eluent: n-heptane/ethanol 1:1; flow rate: 20 ml/min; temperature: 40° C.; detection: 220 nm]. After concentration of the product fractions and drying of the solids under high vacuum, 72 mg (96% of theory) of enantiomer 1 were obtained (99% ee, chiral analytical HPLC).

¹H-NMR (400 MHz, DMSO-d₆, δ/ppm): 11.14 (br. s, 1H), 7.14 (br. s, 1H), 5.30 (d, 1H), 5.13 (s, 2H), 4.34 (t, 2H), 4.18-4.04 (m, 2H), 2.45 (s, 3H), 2.27-2.21 (m, 1H), 1.14 (d, 3H), 1.03-0.91 (m, 1H), 0.86-0.77 (m, 2H).

Chiral analytical HPLC [column: Daicel Chiralpak OX-3, 3 μm 50 mm×4.6 mm; eluent: n-heptane/ethanol 1:1; flow rate: 1 ml/min; temperature: 25° C.; detection: 220 nm]: Rₜ=1.18 min.

### Example 275

### 5-Methyl-3-(2-methylcyclopropyl)-6-[(5-oxo-2,5-dihydro-1H-pyrazol-1-yl)methyl]-1-[2-(trifluoromethoxy)ethyl]thieno[2,3-d]pyrimidine-2,4(1H,3H)-dione (trans enantiomer 2)

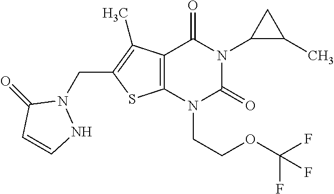 [SMILES: Cc1c(Cn2[nH]ccc2=O)sc2c1c(=O)n(C1CC1C)c(=O)n2CCOC(F)(F)F]

65 mg (87% of theory) of enantiomer 2 were obtained from the preparative HPLC separation on a chiral phase described in Ex. 274 (99% ee, chiral analytical HPLC).

¹H-NMR (400 MHz, DMSO-d₆, δ/ppm): 11.13 (br. s, 1H), 7.14 (br. s, 1H), 5.30 (s, 1H), 5.13 (s, 2H), 4.34 (t, 2H), 4.18-4.04 (m, 2H), 2.45 (s, 3H), 2.27-2.21 (m, 1H), 1.14 (d, 3H), 1.03-0.91 (m, 1H), 0.87-0.76 (m, 2H).

Chiral analytical HPLC [column: Daicel Chiralpak OX-3, 3 μm 50 mm×4.6 mm; eluent: n-heptane/ethanol 1:1; flow rate: 1 ml/min; temperature: 25° C.; detection: 220 nm]: Rₜ=1.60 min.

### Example 276

### 5-Methyl-3-(2-methylcyclopropyl)-6-[(5-oxo-4,5-dihydro-1H-1,2,4-triazol-1-yl)methyl]-1-[2-(trifluoromethoxy)ethyl]thieno[2,3-d]pyrimidine-2,4(1H,3H)-dione (trans racemate)

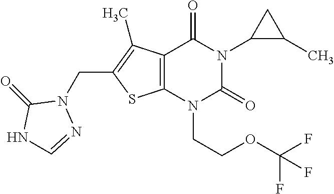 [SMILES: Cc1c(Cn2nc[nH]c2=O)sc2c1c(=O)n(C1CC1C)c(=O)n2CCOC(F)(F)F]

270 mg (0.504 mmol) of the compound from Ex. 450A were dissolved in a mixture of 16.5 ml each of methanol and trimethyl orthoformate, and 1.3 ml (5.04 mmol) of a 4 M solution of hydrogen chloride in dioxane were added at RT. After 4 days, the reaction mixture was concentrated and the remaining residue was purified by means of preparative HPLC (Method 11). After concentration of the product fractions and drying under high vacuum, 136 mg (60% of theory) of the title compound were obtained.

¹H-NMR (400 MHz, DMSO-d₆, δ/ppm): 11.59 (br. s, 1H), 7.82 (s, 1H), 4.92 (s, 2H), 4.36 (t, 2H), 4.21-4.05 (m, 2H), 2.44 (s, 3H), 2.29-2.20 (m, 1H), 1.15 (d, 3H), 1.04-0.92 (m, 1H), 0.87-0.77 (m, 2H).

LC/MS (Method 1, ESIpos): Rₜ=1.48 min, m/z=446.11 [M+H]⁺.

### Example 277

### 5-Methyl-3-(2-methylcyclopropyl)-6-[(5-oxo-4,5-dihydro-1H-1,2,4-triazol-1-yl)methyl]-1-[2-(trifluoromethoxy)ethyl]thieno[2,3-d]pyrimidine-2,4(1H,3H)-dione (trans enantiomer 1)

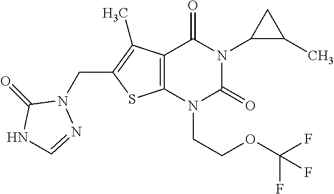 [SMILES: Cc1c(Cn2nc[nH]c2=O)sc2c1c(=O)n(C1CC1C)c(=O)n2CCOC(F)(F)F]

132 mg of the racemic compound from Ex. 276 were dissolved in 20 ml of a methanol/acetonitrile mixture and, in 10 portions, separated into the enantiomers by preparative SFC-HPLC on a chiral phase [column: Daicel Chiralcel OX-H, 5 μm, 250 mm×20 mm; eluent: carbon dioxide/ethanol 83:17; flow rate: 80 ml/min; temperature: 40° C.; detection: 210 nm]. After concentration of the product fractions and drying of the solids under high vacuum, 46 mg (69% of theory) of enantiomer 1 were obtained (>99% ee, chiral analytical HPLC).

¹H-NMR (400 MHz, DMSO-d₆, δ/ppm): 11.58 (br. s, 1H), 7.82 (s, 1H), 4.92 (s, 2H), 4.36 (t, 2H), 4.21-4.05 (m, 2H), 2.44 (s, 3H), 2.28-2.21 (m, 1H), 1.15 (d, 3H), 1.04-0.92 (m, 1H), 0.87-0.78 (m, 2H).

Chiral analytical SFC-HPLC [column: Daicel Chiralpak OX-H 5 μm, 50 mm×4.6 mm; eluent: carbon dioxide/ethanol 80:20; flow rate: 3 ml/min; temperature: 40° C.; detection: 210 nm]: Rₜ=1.81 min.

### Example 278

### 5-Methyl-3-(2-methylcyclopropyl)-6-[(5-oxo-4,5-dihydro-1H-1,2,4-triazol-1-yl)methyl]-1-[2-(trifluoromethoxy)ethyl]thieno[2,3-d]pyrimidine-2,4(1H,3H)-dione (trans enantiomer 2)

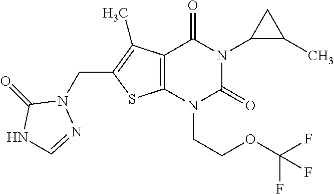 [SMILES: Cc1c(Cn2nc[nH]c2=O)sc2c1c(=O)n(C1CC1C)c(=O)n2CCOC(F)(F)F]

43 mg (65% of theory) of enantiomer 2 were obtained from the preparative HPLC separation on a chiral phase described in Ex. 277 (>99% ee, chiral analytical HPLC).

¹H-NMR (400 MHz, DMSO-d₆, δ/ppm): 11.58 (br. s, 1H), 7.83 (s, 1H), 4.92 (s, 2H), 4.36 (t, 2H), 4.20-4.06 (m, 2H), 2.44 (s, 3H), 2.29-2.21 (m, 1H), 1.15 (d, 3H), 1.04-0.92 (m, 1H), 0.87-0.77 (m, 2H).

Chiral analytical SFC-HPLC [column: Daicel Chiralpak OX-H 5 μm, 50 mm×4.6 mm; eluent: carbon dioxide/ethanol 80:20; flow rate: 3 ml/min; temperature: 40° C.; detection: 210 nm]: Rₜ=3.79 min.

### Example 279

### 5-Methyl-3-(2-methylcyclopropyl)-6-[(2-oxoimidazolidin-1-yl)methyl]-1-{2-[(trifluoromethyl)sulfanyl]ethyl}thieno[2,3-d]pyrimidine-2,4(1H,3H)-dione (trans racemate)

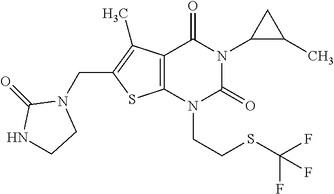 [SMILES: Cc1c(CN2CCNC2=O)sc2c1c(=O)n(C1CC1C)c(=O)n2CCSC(F)(F)F]

To a solution of 294 mg of the crude product from Ex. 379A (0.67 mmol, calculated with a theoretical purity of 100% and a yield of 100%) and 141 μl (1.01 mmol) of triethylamine in 2 ml of THF were added 131 mg (0.81 mmol) of 1,1′-carbonyldiimidazole (CDI), and the mixture was heated to 80° C. in a microwave apparatus (Biotage Initiator) for 5 min. The reaction mixture was then stirred into water and extracted three times with 5 ml each time of ethyl acetate. The combined organic phases were washed with saturated aqueous sodium chloride solution, dried over sodium sulfate, filtered and concentrated on a rotary evaporator. The residue was purified by preparative HPLC (Method 16). After concentration of the product fractions and drying under high vacuum, 70 mg (23% of theory) of the title compound were obtained.

¹H-NMR (400 MHz, DMSO-d₆, δ/ppm): 6.52 (s, 1H), 4.35 (s, 2H), 4.10 (m, 2H), 3.32 (t, 2H), 3.22 (m, 4H), 2.38 (s, 3H), 2.23 (m, 1H), 1.15 (d, 3H), 1.02-0.95 (m, 1H), 0.87-0.80 (m, 2H).

LC/MS (Method 1, ESIpos): Rₜ=1.74 min, m/z=463.1 [M+H]⁺.

### Example 280

### 5-Methyl-3-(2-methylcyclopropyl)-6-[(2-oxoimidazolidin-1-yl)methyl]-1-{2-[(trifluoromethyl)sulfanyl]ethyl}thieno[2,3-d]pyrimidine-2,4(1H,3H)-dione (trans enantiomer 1)

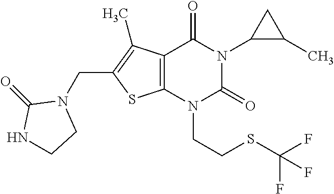 [SMILES: Cc1c(CN2CCNC2=O)sc2c1c(=O)n(C1CC1C)c(=O)n2CCSC(F)(F)F]

65 mg of the racemic compound from Ex. 279 were dissolved in a mixture of 1 ml of ethanol and 3 ml of acetonitrile and separated into the enantiomers via preparative HPLC on a chiral phase [column: Daicel Chiralcel OX-H, 5 μm, 250 mm×20 mm; eluent: n-heptane/ethanol 30:70; flow rate: 15 ml/min; temperature: 25° C.; detection: 220 nm]. After concentration of the product fractions and drying of the solids under high vacuum, 27 mg (42% of theory) of enantiomer 1 were obtained (>99% ee, chiral analytical HPLC).

¹H-NMR (400 MHz, DMSO-d₆, δ/ppm): 6.52 (s, 1H), 4.35 (s, 2H), 4.10 (m, 2H), 3.32 (t, 2H), 3.22 (m, 4H), 2.38 (s, 3H), 2.23 (m, 1H), 1.15 (d, 3H), 1.02-0.95 (m, 1H), 0.87-0.80 (m, 2H).

Chiral analytical HPLC [column: Daicel Chiralpak OX-3, 3 μm 50 mm×4.6 mm; eluent: n-heptane/ethanol 1:1; flow rate: 1 ml/min; detection: 220 nm]: Rₜ=2.02 min.

### Example 281

### 5-Methyl-3-(2-methylcyclopropyl)-6-[(2-oxoimidazolidin-1-yl)methyl]-1-{2-[(trifluoromethyl)sulfanyl]ethyl}thieno[2,3-d]pyrimidine-2,4(1H,3H)-dione (trans enantiomer 2)

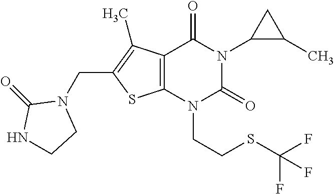 [SMILES: Cc1c(CN2CCNC2=O)sc2c1c(=O)n(C1CC1C)c(=O)n2CCSC(F)(F)F]

65 mg of the racemic compound from Ex. 279 were dissolved in a mixture of 1 ml of ethanol and 3 ml of acetonitrile and separated into the enantiomers via preparative HPLC on a chiral phase [column: Daicel Chiralcel OX-H, 5 μm, 250 mm×20 mm; eluent: n-heptane/ethanol 30:70; flow rate: 15 ml/min; temperature: 25° C.; detection: 220 nm]. After concentration of the product fractions and drying of the solids under high vacuum, 28 mg (43% of theory) of enantiomer 2 were obtained (>99% ee, chiral analytical HPLC).

¹H-NMR (400 MHz, DMSO-d₆, δ/ppm): 6.52 (s, 1H), 4.35 (s, 2H), 4.10 (m, 2H), 3.32 (t, 2H), 3.22 (m, 4H), 2.38 (s, 3H), 2.23 (m, 1H), 1.15 (d, 3H), 1.02-0.95 (m, 1H), 0.87-0.80 (m, 2H).

Chiral analytical HPLC [column: Daicel Chiralpak OX-3, 3 μm 50 mm×4.6 mm; eluent: n-heptane/ethanol 1:1; flow rate: 1 ml/min; detection: 220 nm]: Rₜ=2.97 min.

### Example 282

### 5-Methyl-3-[(1S,2S)-2-methylcyclopropyl]-1-(oxetan-2-ylmethyl)-6-[(2-oxoimidazoliidin-1-yl)methyl]thieno[2,3-d]pyrimidine-2,4(1H,3H)-dione (diastereomer mixture)

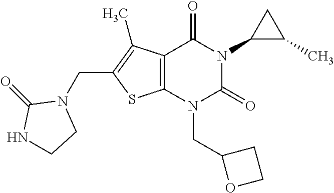 [SMILES: Cc1c(CN2CCNC2=O)sc2c1c(=O)n([C@H]1C[C@@H]1C)c(=O)n2CC1CCO1]

To a solution of 460 mg of the crude product from Ex. 380A (1.22 mmol, calculated with a theoretical purity of 100%) and 254 μl (1.82 mmol) of triethylamine in 10 ml of THF were added 236 mg (1.46 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 161 mg (32% of theory) of the title compound were obtained.

¹H-NMR (400 MHz, DMSO-d₆, δ/ppm): 6.51 (broad, 1H), 4.98 (m, 1H), 4.47 (m, 1H), 4.41 (m, 1H), 4.33 (s, 2H), 4.09 (m, 2H), 3.21 (m, 4H), 2.67 (m, 1H), 2.37 (s, 3H), 2.23 (m, 1H), 1.15 (d, 3H), 1.03-0.95 (m, 1H), 0.86-0.78 (m, 2H).

LC/MS (Method 1, ESIpos): Rₜ=1.25 min, m/z=405.1 [M+H]⁺.

### Example 283

### 5-Methyl-3-[(1S,2S)-2-methylcyclopropyl]-1-(oxetan-2-ylmethyl)-6-[(2-oxoimidazoliidin-1-yl)methyl]thieno[2,3-d]pyrimidine-2,4(1H,3H)-dione (diastereomer 1)

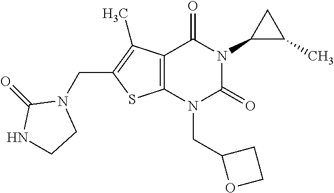 [SMILES: Cc1c(CN2CCNC2=O)sc2c1c(=O)n([C@H]1C[C@@H]1C)c(=O)n2CC1CCO1]

161 mg of the diastereomer mixture from Ex. 282 were dissolved in about 4 ml of methanol/acetonitrile/dichloromethane (3:3:2) and separated into the enantiomerically pure diastereomers via preparative HPLC on a chiral phase [column: Daicel Chiralpak IC, 5 μm, 250 mm×20 mm; eluent: acetonitrile/methanol 1:1; flow rate: 20 ml/min; detection: 220 nm]. After concentration of the product fractions and drying of the solids under high vacuum, 68 mg (42% of theory) of diastereomer 1 were obtained (>99% de, chiral analytical HPLC).

¹H-NMR (400 MHz, DMSO-d₆, δ/ppm): 6.50 (s, 1H), 4.98 (m, 1H), 4.47 (m, 1H), 4.41 (m, 1H), 4.33 (s, 2H), 4.09 (d, 2H), 3.21 (m, 4H), 2.67 (m, 1H), 2.37 (s, 3H), 2.24 (m, 1H), 1.15 (d, 3H), 1.03-0.95 (m, 1H), 0.85-0.79 (m, 2H).

Chiral analytical HPLC [column: Daicel Chiralpak IC, 5 μm, 250 mm×4.6 mm; eluent: acetonitrile/methanol 1:1; flow rate: 1 ml/min; detection: 220 nm]: Rₜ=9.30 min.

### Example 284

### 5-Methyl-3-[(1S,2S)-2-methylcyclopropyl]-1-(oxetan-2-ylmethyl)-6-[(2-oxoimidazolidin-1-yl)methyl]thieno[2,3-d]pyrimidine-2,4(1H,3H)-dione (diastereomer 2)

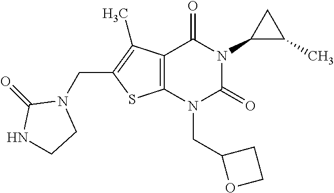 [SMILES: Cc1c(CN2CCNC2=O)sc2c1c(=O)n([C@H]1C[C@@H]1C)c(=O)n2CC1CCO1]

161 mg of the diastereomer mixture from Ex. 282 were dissolved in about 4 ml of methanol/acetonitrile/dichloromethane (3:3:2) and separated into the enantiomerically pure diastereomers via preparative HPLC on a chiral phase [column: Daicel Chiralpak IC, 5 μm, 250 mm×20 mm; eluent: acetonitrile/methanol 1:1; flow rate: 20 ml/min; detection: 220 nm]. After concentration of the product fractions and drying of the solids under high vacuum, 6.2 mg (4% of theory) of diastereomer 2 were obtained (99% de, chiral analytical HPLC).

¹H-NMR (400 MHz, DMSO-d₆, δ/ppm): 6.51 (s, 1H), 4.98 (m, 1H), 4.47 (m, 1H), 4.41 (m, 1H), 4.33 (s, 2H), 4.09 (dq, 2H), 3.22 (m, 4H), 2.67 (m, 1H), 2.37 (s, 3H), 2.24 (m, 1H), 1.15 (d, 3H), 1.02-0.95 (m, 1H), 0.86-0.79 (m, 2H).

Chiral analytical HPLC [column: Daicel Chiralpak IC, 5 μm, 250 mm×4.6 mm; eluent: acetonitrile/methanol 1:1; flow rate: 1 ml/min; detection: 220 nm]: Rₜ=11.54 min.

### Example 285

### 5-Methyl-3-[(1S,2S)-2-methylcyclopropyl]-1-(oxetan-3-ylmethyl)-6-[(2-oxoimidazoliddin-1-yl)methyl]thieno[2,3-d]pyrimidine-2,4(1H,3H)-dione

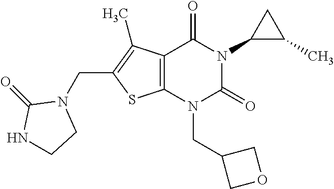 [SMILES: Cc1c(CN2CCNC2=O)sc2c1c(=O)n([C@H]1C[C@@H]1C)c(=O)n2CC1COC1]

To a solution of 113 mg of the crude product from Ex. 381A (0.30 mmol, calculated with a theoretical purity of 100% and a yield of 100%) and 62 μl (0.45 mmol) of triethylamine in 1.7 ml of THF were added 58 mg (0.36 mmol) of 1,1′-carbonyldiimidazole (CDI), and the mixture was heated to 80° C. in a microwave apparatus (Biotage Initiator) for 10 min. The reaction mixture was then separated directly into its components by means of preparative HPLC (Method 16). After concentration of the product fractions and drying under high vacuum, the material thus obtained was repurified by means of another preparative HPLC (Method 17). 5 mg (4% of theory) of the title compound were obtained.

¹H-NMR (400 MHz, DMSO-d₆, δ/ppm): 6.52 (s, 1H), 4.61 (dd, 2H), 4.42 (t, 2H), 4.34 (s, 2H), 4.15 (m, 2H), 3.39 (m, 1H), 3.22 (m, 4H), 2.37 (s, 3H), 2.22 (m, 1H), 1.15 (d, 3H), 1.02-0.92 (m, 1H), 0.85-0.78 (m, 2H).

LC/MS (Method 1, ESIpos): Rₜ=1.20 min, m/z=405.1 [M+H]⁺.

### Example 286

### 5-Methyl-3-(2-methylcyclopropyl)-6-[(2-oxoimidazolidin-1-yl)methyl]-1-[(2R)-tetrahydrofuran-2-ylmethyl]thieno[2,3-d]pyrimidine-2,4(1H,3H)-dione (trans-diastereomer mixture)

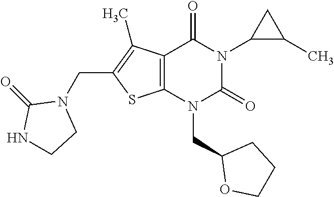 [SMILES: Cc1c(CN2CCNC2=O)sc2c1c(=O)n(C1CC1C)c(=O)n2C[C@H]1CCCO1]

To a solution of 230 mg of the crude product from Ex. 382A (0.58 mmol, calculated with a theoretical purity of 100% and a yield of 100%) and 123 μl (0.88 mmol) of triethylamine in 3.8 ml of THF were added 114 mg (0.70 mmol) of 1,1′-carbonyldiimidazole (CDI), and the mixture was heated to 80° C. in a microwave apparatus (Biotage Initiator) for 5 min. The reaction mixture was then separated directly into its components by means of preparative HPLC (Method 16). After concentration of the product fractions and drying under high vacuum, 100 mg (41% of theory) of the title compound were obtained.

¹H-NMR (400 MHz, DMSO-d₆, δ/ppm): 6.51 (broad, 1H), 4.33 (s, 2H), 4.20 (broad, 1H), 4.00 (m, 1H), 3.77-3.58 (m, 3H), 3.21 (m, 4H), 2.37 (s, 3H), 2.24 (m, 1H), 2.01-1.75 (m, 3H), 1.64 (m, 1H), 1.15 (d, 3H), 1.02-0.92 (m, 1H), 0.85-0.79 (m, 2H).

LC/MS (Method 1, ESIpos): Rₜ=1.42 min, m/z=419.1 [M+H]⁺.

### Example 287

### 5-Methyl-3-(2-methylcyclopropyl)-6-[(2-oxoimidazolidin-1-yl)methyl]-1-[(2R)-tetrahydrofuran-2-ylmethyl]thieno[2,3-d]pyrimidine-2,4(1H,3H)-dione (trans diastereomer 1)

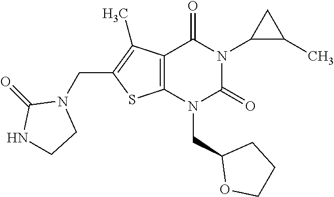 [SMILES: Cc1c(CN2CCNC2=O)sc2c1c(=O)n(C1CC1C)c(=O)n2C[C@H]1CCCO1]

94 mg of the diastereomer mixture from Ex. 286 were dissolved in 4 ml of ethanol and 1 ml of acetonitrile and separated into the enantiomerically pure diastereomers via preparative HPLC on a chiral phase [column: Daicel Chiralcel OJ-H, 5 μm, 250 mm×20 mm; eluent: n-heptane/ethanol 1:1; flow rate: 20 ml/min; temperature: 40° C.; detection: 220 nm]. After concentration of the product fractions and drying of the solids under high vacuum, 30 mg (32% of theory) of diastereomer 1 were obtained (>99% de, chiral analytical HPLC).

¹H-NMR (400 MHz, DMSO-d₆, δ/ppm): 6.50 (s, 1H), 4.33 (s, 2H), 4.20 (broad, 1H), 4.01 (dd, 1H), 3.75 (q, 1H), 3.66-3.58 (m, 2H), 3.21 (m, 4H), 2.37 (s, 3H), 2.24 (m, 1H), 2.01-1.77 (m, 3H), 1.64 (m, 1H), 1.15 (d, 3H), 1.02-0.92 (m, 1H), 0.85-0.79 (m, 2H).

Chiral analytical HPLC [column: Daicel OJ-3, 3 μm 50 mm×4.6 mm; eluent: ethanol; flow rate: 1 ml/min; detection: 220 nm]: Rₜ=1.56 min.

### Example 288

### 5-Methyl-3-(2-methylcyclopropyl)-6-[(2-oxoimidazolidin-1-yl)methyl]-1-[(2R)-tetrahydrofuran-2-ylmethyl]thieno[2,3-d]pyrimidine-2,4(1H,3H)-dione (trans diastereomer 2)

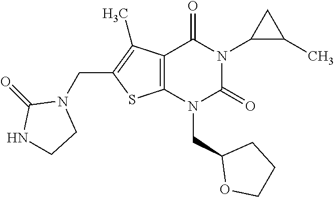 [SMILES: Cc1c(CN2CCNC2=O)sc2c1c(=O)n(C1CC1C)c(=O)n2C[C@H]1CCCO1]

94 mg of the diastereomer mixture from Ex. 286 were dissolved in 4 ml of ethanol and 1 ml of acetonitrile and separated into the enantiomerically pure diastereomers via preparative HPLC on a chiral phase [column: Daicel Chiralcel OJ-H, 5 μm, 250 mm×20 mm; eluent: n-heptane/ethanol 1:1; flow rate: 20 ml/min; temperature: 40° C.; detection: 220 nm]. After concentration of the product fractions and drying of the solids under high vacuum, 40 mg (43% of theory) of diastereomer 2 were obtained (98.6% de, chiral analytical HPLC).

¹H-NMR (400 MHz, DMSO-d₆, δ/ppm): 6.50 (s, 1H), 4.33 (s, 2H), 4.20 (broad, 1H), 3.99 (dd, 1H), 3.77-3.58 (m, 3H), 3.21 (m, 4H), 2.37 (s, 3H), 2.24 (m, 1H), 2.01-1.77 (m, 3H), 1.64 (m, 1H), 1.15 (d, 3H), 1.02-0.92 (m, 1H), 0.85-0.79 (m, 2H).

Chiral analytical HPLC [column: Daicel OJ-3, 3 μm 50 mm×4.6 mm; eluent: ethanol; flow rate: 1 ml/min; detection: 220 nm]: Rₜ=2.17 min.

### Example 289

### 5-Methyl-3-(2-methylcyclopropyl)-6-[(2-oxoimidazolidin-1-yl)methyl]-1-(tetrahydrofuran-3-ylmethyl)thieno[2,3-d]pyrimidine-2,4(1H,3H)-dione (mixture of all trans stereoisomers)

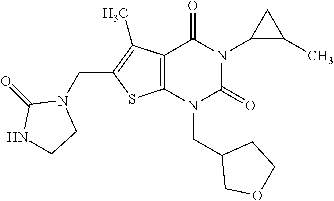 [SMILES: Cc1c(CN2CCNC2=O)sc2c1c(=O)n(C1CC1C)c(=O)n2CC1CCOC1]

Analogously to the process described in Ex. 286, 297 mg of the crude product from Ex. 383A (0.76 mmol, calculated with theoretical purity of 100% and a yield of 100%) were used to obtain 107 mg (32% of theory) of the title compound.

¹H-NMR (400 MHz, DMSO-d₆, δ/ppm): 6.52 (s, 1H), 4.34 (s, 2H), 3.88-3.77 (m, 3H), 3.63 (m, 2H), 3.50 (m, 1H), 3.10 (q, 2H), 2.71 (m, 1H), 2.38 (s, 3H), 2.24 (m, 1H), 1.94 (m, 1H), 1.65 (m, 1H), 1.15 (d, 3H), 1.02-0.94 (m, 1H), 0.85-0.80 (m, 2H).

LC/MS (Method 1, ESIpos): Rₜ=1.31 min, m/z=419.1 [M+H]⁺.

### Example 290

### 5-Methyl-3-(2-methylcyclopropyl)-6-[(2-oxoimidazolidin-1-yl)methyl]-1-(tetrahydrofuran-3-ylmethyl)thieno[2,3-d]pyrimidine-2,4(1H,3H)-dione (trans stereoisomer 1)

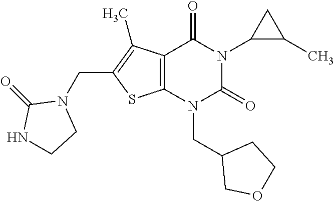 [SMILES: Cc1c(CN2CCNC2=O)sc2c1c(=O)n(C1CC1C)c(=O)n2CC1CCOC1]

100 mg (0.24 mmol) of the diastereomer mixture from Ex. 289 were dissolved in 6 ml of ethanol and first separated into two respective diastereomer pairs via preparative HPLC on a chiral phase [column: YMC Chiralart Cellulose SC, 5 μm, 250 mm×20 mm; eluent: ethanol; flow rate: 15 ml/min; temperature: 60° C.; detection: 220 nm]. After concentration of the corresponding product fractions, the diastereomer pairs were then separated into the individual diastereomers via a further preparative HPLC on a chiral phase [column: Daicel Chiralcel OX-H, 5 μm, 250 mm×20 mm; eluent: isopropanol; flow rate: 15 ml/min; temperature: 70° C.; detection: 220 nm]. After concentration of the product fractions and drying of the solids under high vacuum, 16 mg (16% of theory) of diastereomer 1 were obtained (>99% de, chiral analytical HPLC).

¹H-NMR (400 MHz, DMSO-d₆, δ/ppm): 6.52 (s, 1H), 4.34 (s, 2H), 3.88-3.77 (m, 3H), 3.63 (m, 2H), 3.50 (m, 1H), 3.22 (m, 4H), 2.71 (m, 1H), 2.38 (s, 3H), 2.24 (m, 1H), 1.94 (m, 1H), 1.63 (m, 1H), 1.15 (d, 3H), 1.03-0.94 (m, 1H), 0.86-0.80 (m, 2H).

Chiral analytical HPLC [column: Daicel Chiralcel OX-H, 5 μm, 250 mm×4.6 mm; eluent: isopropanol; flow rate: 1 ml/min; detection: 220 nm]: Rₜ=8.16 min.

### Example 291

### 5-Methyl-3-(2-methylcyclopropyl)-6-[(2-oxoimidazolidin-1-yl)methyl]-1-(tetrahydrofuran-3-ylmethyl)thieno[2,3-d]pyrimidine-2,4(1H,3H)-dione (trans stereoisomer 2)

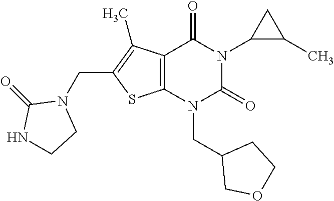 [SMILES: Cc1c(CN2CCNC2=O)sc2c1c(=O)n(C1CC1C)c(=O)n2CC1CCOC1]

16 mg (16% of theory) of diastereomer 2 were obtained from the double HPLC separation described in Ex. 290 (>99% de, chiral analytical HPLC).

¹H-NMR (400 MHz, DMSO-d₆, δ/ppm): 6.51 (s, 1H), 4.34 (s, 2H), 3.90-3.77 (m, 3H), 3.63 (m, 2H), 3.50 (m, 1H), 3.22 (m, 4H), 2.71 (m, 1H), 2.38 (s, 3H), 2.23 (m, 1H), 1.94 (m, 1H), 1.64 (m, 1H), 1.15 (d, 3H), 1.03-0.94 (m, 1H), 0.86-0.80 (m, 2H).

Chiral analytical HPLC [column: Daicel Chiralcel OX-H, 5 μm, 250 mm×4.6 mm; eluent: isopropanol; flow rate: 1 ml/min; detection: 220 nm]: Rₜ=9.45 min.

### Example 292

### 5-Methyl-3-(2-methylcyclopropyl)-6-[(2-oxoimidazolidin-1-yl)methyl]-1-(tetrahydrofuran-3-ylmethyl)thieno[2,3-d]pyrimidine-2,4(1H,3H)-dione (trans stereoisomer 3)

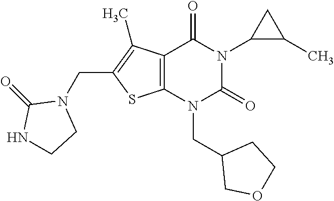 [SMILES: Cc1c(CN2CCNC2=O)sc2c1c(=O)n(C1CC1C)c(=O)n2CC1CCOC1]

17 mg (17% of theory) of diastereomer 3 were obtained from the double HPLC separation described in Ex. 290 (>99% de, chiral analytical HPLC).

¹H-NMR (400 MHz, DMSO-d₆, δ/ppm): 6.51 (s, 1H), 4.34 (s, 2H), 3.90-3.77 (m, 3H), 3.63 (m, 2H), 3.50 (m, 1H), 3.22 (m, 4H), 2.71 (m, 1H), 2.38 (s, 3H), 2.23 (m, 1H), 1.95 (m, 1H), 1.65 (m, 1H), 1.15 (d, 3H), 1.03-0.94 (m, 1H), 0.86-0.80 (m, 2H).

Chiral analytical HPLC [column: Daicel Chiralcel OX-H, 5 μm, 250 mm×4.6 mm; eluent: isopropanol; flow rate: 1 ml/min; detection: 220 nm]: Rₜ=10.50 min.

### Example 293

### 5-Methyl-3-(2-methylcyclopropyl)-6-[(2-oxoimidazolidin-1-yl)methyl]-1-(tetrahydrofuran-3-ylmethyl)thieno[2,3-d]pyrimidine-2,4(1H,3H)-dione (trans stereoisomer 4)

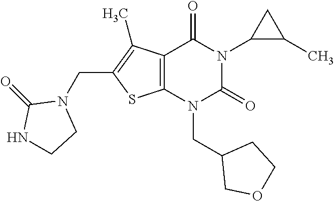 [SMILES: Cc1c(CN2CCNC2=O)sc2c1c(=O)n(C1CC1C)c(=O)n2CC1CCOC1]

17 mg (17% of theory) of diastereomer 4 were obtained from the double HPLC separation described in Ex. 290 (>99% de, chiral analytical HPLC).

¹H-NMR (400 MHz, DMSO-d₆, δ/ppm): 6.51 (s, 1H), 4.34 (s, 2H), 3.85-3.77 (m, 3H), 3.63 (m, 2H), 3.50 (m, 1H), 3.22 (m, 4H), 2.71 (m, 1H), 2.38 (s, 3H), 2.23 (m, 1H), 1.95 (m, 1H), 1.65 (m, 1H), 1.15 (d, 3H), 1.03-0.94 (m, 1H), 0.86-0.80 (m, 2H).

Chiral analytical HPLC [column: Daicel Chiralcel OX-H, 5 μm, 250 mm×4.6 mm; eluent: isopropanol; flow rate: 1 ml/min; detection: 220 nm]: Rₜ=12.15 min.

### Example 294

### 1-(2-Methoxyethyl)-5-methyl-6-[(2-oxoimidazolidin-1-yl)methyl]-3-[2-(trifluoromethyl)cyclopropyl]thieno[2,3-d]pyrimidine-2,4(1H,3H)-dione (trans racemate)

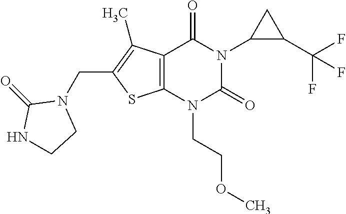 [SMILES: COCCn1c(=O)n(C2CC2C(F)(F)F)c(=O)c2c(C)c(CN3CCNC3=O)sc21]

To a solution of 411 mg (0.860 mmol, 88% purity) of the compound from Ex. 384A and 180 μl (1.29 mmol) of triethylamine in 12 ml of THF were added 167 mg (1.03 mmol) of CDI, and the mixture was stirred at RT for about 16 h. Subsequently, the mixture was concentrated to dryness. The remaining residue was taken up in ethyl acetate and washed successively with water and saturated sodium chloride solution. After drying over anhydrous magnesium sulfate, the mixture was filtered and the solution was concentrated to about half the original volume. In the course of this, a portion of the product precipitated out, which was filtered off. The filtrate was then concentrated to complete dryness and the residue that remained was purified by preparative HPLC (Method 11). After the product fractions had been concentrated, these were combined with the precipitate isolated beforehand and dried under high vacuum. This gave 197 mg (51% of theory) of the title compound.

¹H-NMR (400 MHz, DMSO-d₆, δ/ppm): 6.52 (s, 1H), 4.34 (s, 2H), 4.07-3.90 (m, 2H), 3.61 (t, 2H), 3.28-3.16 (m, 4H), 3.24 (s, 3H), 3.02-2.94 (m, 1H), 2.37 (s, 3H), 2.30-2.16 (m, 1H), 1.49 (q, 1H), 1.37-1.27 (m, 1H).

LC/MS (Method 1, ESIpos): Rₜ=1.47 min, m/z=447.13 [M+H]⁺.

### Example 295

### 1-(2-Methoxyethyl)-5-methyl-6-[(2-oxoimidazolidin-1-yl)methyl]-3-[2-(trifluoromethyl)cyclopropyl]thieno[2,3-d]pyrimidine-2,4(1H,3H)-dione (trans enantiomer 1)

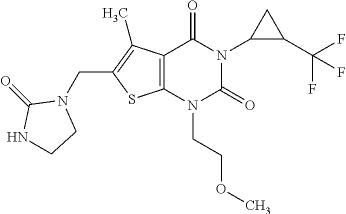 [SMILES: COCCn1c(=O)n(C2CC2C(F)(F)F)c(=O)c2c(C)c(CN3CCNC3=O)sc21]

192 mg of the racemic compound from Ex. 294 were dissolved in 20 ml of acetonitrile and, in 8 portions, separated into the enantiomers by preparative SFC-HPLC on a chiral phase [column: Daicel Chiralcel OX-H, 5 μm, 250 mm×20 mm; eluent: carbon dioxide/ethanol 70:30; flow rate: 80 ml/min; temperature: 40° C.; detection: 210 nm]. After concentration of the product fractions and drying of the solids under high vacuum, 66 mg (68% of theory) of enantiomer 1 were obtained (>99% ee, chiral analytical HPLC).

¹H-NMR (400 MHz, DMSO-d₆, δ/ppm): 6.52 (s, 1H), 4.34 (s, 2H), 4.07-3.90 (m, 2H), 3.61 (t, 2H), 3.28-3.16 (m, 4H), 3.24 (s, 3H), 3.02-2.93 (m, 1H), 2.37 (s, 3H), 2.30-2.17 (m, 1H), 1.49 (q, 1H), 1.38-1.27 (m, 1H).

Chiral analytical SFC-HPLC [column: Daicel Chiralpak OX-H 5 μm, 50 mm×4.6 mm; eluent: carbon dioxide/ethanol 70:30; flow rate: 3 ml/min; temperature: 40° C.; detection: 210 nm]: Rₜ=2.47 min.

### Example 296

### 1-(2-Methoxyethyl)-5-methyl-6-[(2-oxoimidazolidin-1-yl)methyl]-3-[2-(trifluoromethyl)cyclopropyl]thieno[2,3-d]pyrimidine-2,4(1H,3H)-dione (trans enantiomer 2)

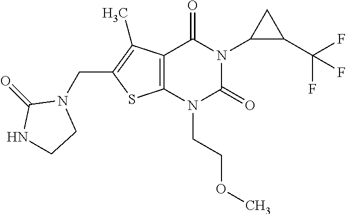 [SMILES: COCCn1c(=O)n(C2CC2C(F)(F)F)c(=O)c2c(C)c(CN3CCNC3=O)sc21]

64 mg (66% of theory) of enantiomer 2 were obtained from the preparative HPLC separation on a chiral phase described in Ex. 295 (>99% ee, chiral analytical HPLC).

¹H-NMR (400 MHz, DMSO-d₆, δ/ppm): 6.52 (s, 1H), 4.34 (s, 2H), 4.07-3.90 (m, 2H), 3.61 (t, 2H), 3.27-3.16 (m, 4H), 3.24 (s, 3H), 2.97 (ddd, 1H), 2.37 (s, 3H), 2.24 (dtd, 1H), 1.55-1.44 (m, 1H), 1.38-1.28 (m, 1H).

Chiral analytical SFC-HPLC [column: Daicel Chiralpak OX-H 5 μm, 50 mm×4.6 mm; eluent: carbon dioxide/ethanol 70:30; flow rate: 3 ml/min; temperature: 40° C.; detection: 210 nm]: Rₜ=5.13 min.

### Example 297

### 1-(2-Methoxyethyl)-5-methyl-6-[(5-oxo-4,5-dihydro-1H-1,2,4-triazol-1-yl)methyl]-3-[2-(trifluoromethyl)cyclopropyl]thieno[2,3-d]pyrimidine-2,4(1H,3H)-dione (trans racemate)

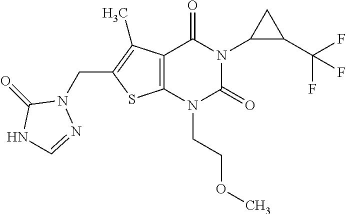 [SMILES: COCCn1c(=O)n(C2CC2C(F)(F)F)c(=O)c2c(C)c(Cn3nc[nH]c3=O)sc21]

Analogously to the method described in Ex. 195, 284 mg (0.504 mmol, 95% purity) of the compound from Ex. 451A were used to prepare 171 mg (76% of theory) of the title compound. The reaction time here was 7 days.

¹H-NMR (400 MHz, DMSO-d₆, δ/ppm): 11.59 (s, 1H), 7.83 (s, 1H), 4.92 (s, 2H), 4.06-3.89 (m, 2H), 3.60 (t, 2H), 3.23 (s, 3H), 2.97 (ddd, 1H), 2.43 (s, 3H), 2.23 (dtd, 1H), 1.55-1.43 (m, 1H), 1.36-1.27 (m, 1H).

LC/MS (Method 1, ESIpos): Rₜ=1.37 min, m/z=446.11 [M+H]⁺.

### Example 298

### 1-(2-Methoxyethyl)-5-methyl-6-[(5-oxo-4,5-dihydro-1H-1,2,4-triazol-1-yl)methyl]-3-[2-(trifluoromethyl)cyclopropyl]thieno[2,3-d]pyrimidine-2,4(1H,3H)-dione (trans enantiomer 1)

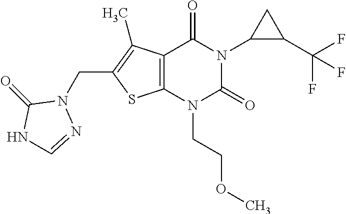 [SMILES: COCCn1c(=O)n(C2CC2C(F)(F)F)c(=O)c2c(C)c(Cn3nc[nH]c3=O)sc21]

165 mg of the racemic compound from Ex. 297 were dissolved in 40 ml of a acetonitrile/methanol mixture and, in 10 portions, separated into the enantiomers by preparative SFC-HPLC on a chiral phase [column: Daicel Chiralcel OX-H, 5 μm, 250 mm×20 mm; eluent: carbon dioxide/ethanol 3:1; flow rate: 90 ml/min; temperature: 40° C.; detection: 210 nm]. After concentration of the product fractions and drying of the solids under high vacuum, 71 mg (86% of theory) of enantiomer 1 were obtained (>99% ee, chiral analytical HPLC).

¹H-NMR (400 MHz, DMSO-d₆, δ/ppm): 11.58 (br. s, 1H), 7.83 (s, 1H), 4.92 (s, 2H), 4.06-3.89 (m, 2H), 3.60 (t, 2H), 3.23 (s, 3H), 3.02-2.92 (m, 1H), 2.43 (s, 3H), 2.28-2.16 (m, 1H), 1.49 (q, 1H), 1.32 (dt, 1H).

Chiral analytical SFC-HPLC [column: Daicel Chiralpak OX-H 5 μm, 50 mm×4.6 mm; eluent: carbon dioxide/ethanol 70:30; flow rate: 3 ml/min; temperature: 40° C.; detection: 210 nm]: Rₜ=1.26 min.

### Example 299

### 1-(2-Methoxyethyl)-5-methyl-6-[(5-oxo-4,5-dihydro-H-1,2,4-triazol-1-yl)methyl]-3-[2-(trifluoromethyl)cyclopropyl]thieno[2,3-d]pyrimidine-2,4(1H,3H)-dione (trans enantiomer 2)

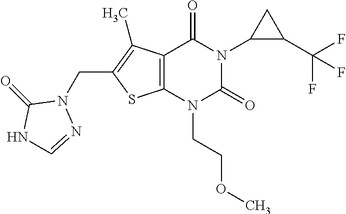 [SMILES: COCCn1c(=O)n(C2CC2C(F)(F)F)c(=O)c2c(C)c(Cn3nc[nH]c3=O)sc21]

83 mg (100% of theory) of enantiomer 2 were obtained from the preparative HPLC separation on a chiral phase described in Ex. 298 (>99% ee, chiral analytical HPLC).

¹H-NMR (400 MHz, DMSO-d₆, δ/ppm): 11.59 (br. s, 1H), 7.83 (s, 1H), 4.92 (s, 2H), 4.06-3.89 (m, 2H), 3.60 (t, 2H), 3.23 (s, 3H), 3.02-2.92 (m, 1H), 2.43 (s, 3H), 2.28-2.17 (m, 1H), 1.49 (q, 1H), 1.37-1.27 (m, 1H).

Chiral analytical SFC-HPLC [column: Daicel Chiralpak OX-H 5 μm, 50 mm×4.6 mm; eluent: carbon dioxide/ethanol 70:30; flow rate: 3 ml/min; temperature: 40° C.; detection: 210 nm]: Rₜ=1.95 min.

### Example 300

### 3-(2-Ethylcyclopropyl)-1-(2-methoxyethyl)-5-methyl-6-[(2-oxoimidazolidin-1-yl)methyl]thieno[2,3-d]pyrimidine-2,4(1H,3H)-dione (trans racemate)

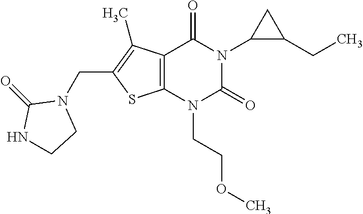 [SMILES: CCC1CC1n1c(=O)c2c(C)c(CN3CCNC3=O)sc2n(CCOC)c1=O]

Analogously to the method described in Ex. 294, 323 mg (0.696 mmol, 82% purity) of the compound from Ex. 385A and 135 mg (0.835 mmol) of CDI were used to prepare 93 mg (32% of theory) of the title compound.

¹H-NMR (400 MHz, DMSO-d₆, δ/ppm): 6.51 (s, 1H), 4.33 (s, 2H), 4.04-3.89 (m, 2H), 3.60 (t, 2H), 3.27-3.14 (m, 4H), 3.23 (s, 3H), 2.37 (s, 3H), 2.30 (dt, 1H), 1.69-1.55 (m, 1H), 1.30-1.16 (m, 1H), 1.07-0.92 (m, 1H), 0.99 (t, 3H), 0.89-0.71 (m, 2H).

LC/MS (Method 1, ESIpos): Rₜ=1.49 min, m/z=407.17 [M+H]⁺.

### Example 301

### 3-(2-Ethylcyclopropyl)-1-(2-methoxyethyl)-5-methyl-6-[(2-oxoimidazolidin-1-yl)methyl]thieno[2,3-d]pyrimidine-2,4(1H,3H)-dione (trans enantiomer 1)

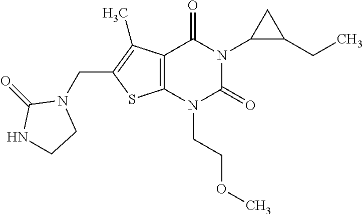 [SMILES: CCC1CC1n1c(=O)c2c(C)c(CN3CCNC3=O)sc2n(CCOC)c1=O]

90 mg of the racemic compound from Ex. 300 were dissolved in a mixture of 3 ml of acetonitrile and 2 ml of ethanol and, in 13 portions, separated into the enantiomers via preparative HPLC on a chiral phase [column: Daicel Chiralcel OX-H, 5 μm, 250 mm×20 mm; eluent: n-heptane/ethanol 90:10; flow rate: 15 ml/min; temperature: 40° C.; detection: 210 nm]. After concentration of the product fractions and drying of the solids under high vacuum, 39 mg (86% of theory) of enantiomer 1 were obtained (>99% ee, chiral analytical HPLC).

¹H-NMR (400 MHz, DMSO-d₆, δ/ppm): 6.51 (s, 1H), 4.33 (s, 2H), 3.97 (td, 2H), 3.60 (t, 2H), 3.27-3.16 (m, 4H), 3.23 (s, 3H), 2.37 (s, 3H), 2.30 (dt, 1H), 1.70-1.55 (m, 1H), 1.29-1.15 (m, 1H), 1.06-0.94 (m, 1H), 0.99 (t, 3H), 0.88-0.73 (m, 2H).

Chiral analytical HPLC [column: Daicel Chiralpak OX-3, 3 μm 50 mm×4.6 mm; eluent: ethanol; flow rate: 1 ml/min; temperature: 50° C.; detection: 220 nm]: Rₜ=1.65 min.

### Example 302

### 3-(2-Ethylcyclopropyl)-1-(2-methoxyethyl)-5-methyl-6-[(2-oxoimidazolidin-1-yl)methyl]thieno[2,3-d]pyrimidine-2,4(1H,3H)-dione (trans enantiomer 2)

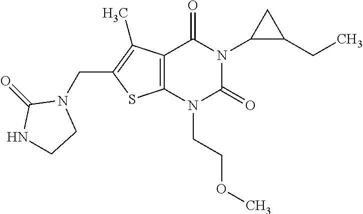 [SMILES: CCC1CC1n1c(=O)c2c(C)c(CN3CCNC3=O)sc2n(CCOC)c1=O]

40 mg (88% of theory) of enantiomer 2 were obtained from the preparative HPLC separation on a chiral phase described in Ex. 301 (>99% ee, chiral analytical HPLC).

¹H-NMR (400 MHz, DMSO-d₆, δ/ppm): 6.51 (s, 1H), 4.33 (s, 2H), 3.97 (td, 2H), 3.60 (t, 2H), 3.27-3.15 (m, 4H), 3.23 (s, 3H), 2.37 (s, 3H), 2.34-2.27 (m, 1H), 1.68-1.55 (m, 1H), 1.29-1.16 (m, 1H), 1.07-0.94 (m, 1H), 0.99 (t, 3H), 0.88-0.74 (m, 2H).

Chiral analytical HPLC [column: Daicel Chiralpak OX-3, 3 μm 50 mm×4.6 mm; eluent: ethanol; flow rate: 1 ml/min; temperature: 50° C.; detection: 220 nm]: Rₜ=3.02 min.

### Example 303

### 3-(2-Ethylcyclopropyl)-1-(2-methoxyethyl-6-[(5-oxo-4,5-dihydro-1H-1,2,4-triazol-1-yl)methyl]thieno[2,3-d]pyrimidine-2,4(1H,3H)-dione (trans racemate)

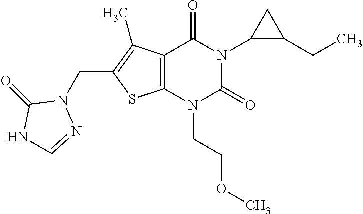 [SMILES: CCC1CC1n1c(=O)c2c(C)c(Cn3nc[nH]c3=O)sc2n(CCOC)c1=O]

340 mg (0.652 mmol, 95% purity) of the compound from Ex. 452A were dissolved in a mixture of 15 ml each of methanol and trimethyl orthoformate, and 2.4 ml (9.78 mmol) of a 4 M solution of hydrogen chloride in dioxane were added at RT. After about 16 hours, the reaction mixture was concentrated to half of the original volume, in the course of which solids are separated out. The precipitation was completed by adding 15 ml of diethyl ether. After stirring at RT for 10 min, the solids were filtered off with suction and dried under reduced pressure. Subsequently, they were stirred in a mixture of 5 ml of DMSO and 10 ml of water at 60° C. for 30 min. After filtration with suction again and drying under high vacuum, 175 mg (66% of theory) of the title compound were obtained.

¹H-NMR (500 MHz, DMSO-d₆, δ/ppm): 11.58 (broad, 1H), 7.82 (s, 1H), 4.91 (s, 2H), 4.03-3.89 (m, 2H), 3.59 (br. t, 2H), 3.22 (s, 3H), 2.43 (s, 3H), 2.30 (dt, 1H), 1.62 (dquin, 1H), 1.22 (dquin, 1H), 0.99 (br. t, 4H), 0.88-0.72 (m, 2H).

LC/MS (Method 2, ESIpos): Rₜ=0.76 min, m/z=406 [M+H]⁺.

### Example 304

### 3-(2-Ethylcyclopropyl)-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione (trans enantiomer 1)

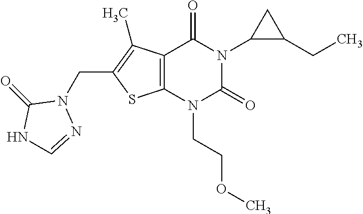 [SMILES: CCC1CC1n1c(=O)c2c(C)c(Cn3nc[nH]c3=O)sc2n(CCOC)c1=O]

168 mg of the racemic compound from Ex. 303 were dissolved in a mixture of 8 ml of dichloromethane and 2 ml of ethanol and, in 20 portions, separated into the enantiomers via preparative HPLC on a chiral phase [column: Daicel Chiralcel OX-H, 5 μm, 250 mm×20 mm; eluent: n-heptane/ethanol 60:40; flow rate: 15 ml/min; temperature: 40° C.; detection: 210 nm]. After concentration of the product fractions and drying of the solids under high vacuum, 73 mg (86% of theory) of enantiomer 1 were obtained (>99% ee, chiral analytical HPLC).

¹H-NMR (500 MHz, DMSO-d₆, δ/ppm): 11.58 (br. s, 1H), 7.83 (s, 1H), 4.91 (s, 2H), 4.02-3.90 (m, 2H), 3.59 (t, 2H), 3.22 (s, 3H), 2.43 (s, 3H), 2.30 (dt, 1H), 1.68-1.55 (m, 1H), 1.22 (dquin, 1H), 1.04-0.94 (m, 1H), 0.99 (t, 3H), 0.87-0.73 (m, 2H).

Chiral analytical HPLC [column: Daicel Chiralpak OX-3, 3 μm 50 mm×4.6 mm; eluent: ethanol; flow rate: 1 ml/min; temperature: 50° C.; detection: 220 nm]: Rₜ=2.69 min.

### Example 305

### 3-(2-Ethylcyclopropyl)-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione (trans enantiomer 2)

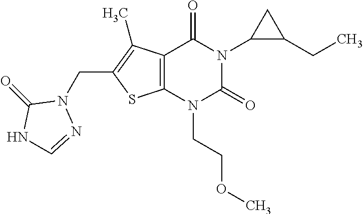 [SMILES: CCC1CC1n1c(=O)c2c(C)c(Cn3nc[nH]c3=O)sc2n(CCOC)c1=O]

74 mg (88% of theory) of enantiomer 2 were obtained from the preparative HPLC separation on a chiral phase described in Ex. 304 (>99% ee, chiral analytical HPLC).

¹H-NMR (500 MHz, DMSO-d₆, δ/ppm): 11.58 (br. s, 1H), 7.83 (s, 1H), 4.91 (s, 2H), 4.02-3.89 (m, 2H), 3.59 (t, 2H), 3.22 (s, 3H), 2.43 (s, 3H), 2.30 (dt, 1H), 1.69-1.55 (m, 1H), 1.22 (dquin, 1H), 1.04-0.93 (m, 1H), 0.99 (t, 3H), 0.86-0.73 (m, 2H).

Chiral analytical HPLC [column: Daicel Chiralpak OX-3, 3 μm 50 mm×4.6 mm; eluent: ethanol; flow rate: 1 ml/min; temperature: 50° C.; detection: 220 nm]: Rₜ=4.71 min.

### Example 306

### 3-(2-Methoxycyclopropyl)-1-(2-methoxyethyl)-5-methyl-6-[(2-oxoimidazolidin-1-yl)methyl]thieno[2,3-d]pyrimidine-2,4(1H,3H)-dione (trans racemate)

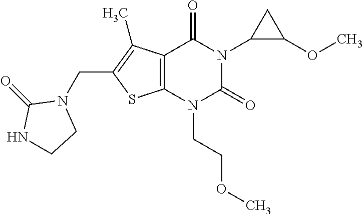 [SMILES: COCCn1c(=O)n(C2CC2OC)c(=O)c2c(C)c(CN3CCNC3=O)sc21]

Analogously to the method described in Ex. 3, 509 mg (0.932 mmol, 70% purity) of the compound from Ex. 386A and 196 mg (1.21 mmol) of CDI were used to prepare 196 mg (51% of theory) of the title compound.

¹H-NMR (500 MHz, DMSO-d₆, δ/ppm): 6.51 (s, 1H), 4.34 (s, 2H), 4.08-3.90 (m, 2H), 3.66-3.55 (m, 2H), 3.41 (ddd, 1H), 3.30-3.16 (m, 10H), 2.60 (dt, 1H), 2.37 (s, 3H), 1.31 (td, 1H), 0.91 (ddd, 1H).

LC/MS (Method 2, ESIpos): Rₜ=0.59 min, m/z=409 [M+H]⁺.

### Example 307

### 3-(2-Methoxycyclopropyl)-1-(2-methoxyethyl)-5-methyl-6-[(2-oxoimidazolidin-1-yl)methyl]thieno[2,3-d]pyrimidine-2,4(1H,3H)-dione (trans enantiomer 1)

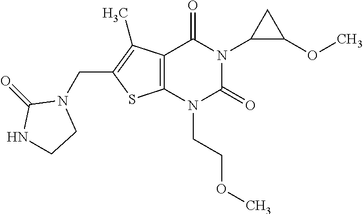 [SMILES: COCCn1c(=O)n(C2CC2OC)c(=O)c2c(C)c(CN3CCNC3=O)sc21]

191 mg of the racemic compound from Ex. 306 were dissolved in a mixture of 6 ml of acetonitrile and 2 ml of ethanol and, in 32 portions, separated into the enantiomers via preparative HPLC on a chiral phase [column: Daicel Chiralcel OD-H, 5 μm 250 mm×20 mm, eluent: n-heptane/ethanol 20:80; flow rate: 15 ml/min; temperature: 40° C.; detection: 210 nm]. After concentration of the product fractions and drying of the solids under high vacuum, 84 mg (87% of theory) of enantiomer 1 were obtained (>99% ee, chiral analytical HPLC).

¹H-NMR (500 MHz, DMSO-d₆, δ/ppm): 6.51 (s, 1H), 4.34 (s, 2H), 4.08-3.90 (m, 2H), 3.66-3.55 (m, 2H), 3.41 (ddd, 1H), 3.28-3.17 (m, 10H), 2.60 (dt, 1H), 2.37 (s, 3H), 1.31 (td, 1H), 0.90 (ddd, 1H).

Chiral analytical HPLC [column: Phenomenex Cellulose 2, 3 μm, 50 mm×4.6 mm; eluent: isohexane/ethanol 1:1; flow rate: 1 ml/min; temperature: 50° C.; detection: 220 nm]: Rₜ=1.95 min.

### Example 308

### 3-(2-Methoxycyclopropyl)-1-(2-methoxyethyl)-5-methyl-6-[(2-oxoimidazolidin-1-yl)methyl]thieno[2,3-d]pyrimidine-2,4(1H,3H)-dione (trans enantiomer 2)

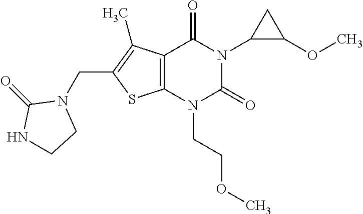 [SMILES: COCCn1c(=O)n(C2CC2OC)c(=O)c2c(C)c(CN3CCNC3=O)sc21]

83 mg (86% of theory) of enantiomer 2 were obtained from the preparative HPLC separation on a chiral phase described in Ex. 307 (>99% ee, chiral analytical HPLC).

¹H-NMR (600 MHz, DMSO-d₆, δ/ppm): 6.49 (s, 1H), 4.33 (s, 2H), 4.07-3.90 (m, 2H), 3.65-3.57 (m, 2H), 3.44-3.38 (m, 1H), 3.28-3.18 (m, 10H), 2.60 (dt, 1H), 2.37 (s, 3H), 1.35-1.26 (m, 1H), 0.91 (ddd, 1H).

Chiral analytical HPLC [column: Phenomenex Cellulose 2, 3 μm, 50 mm×4.6 mm; eluent: isohexane/ethanol 1:1; flow rate: 1 ml/min; temperature: 50° C.; detection: 220 nm]: Rₜ=3.26 min.

### Example 309

### 3-(2-Methoxycyclopropyl)-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione (trans racemate)

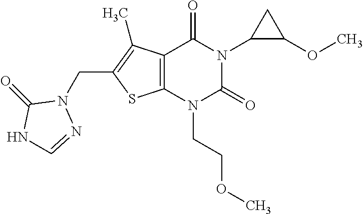 [SMILES: COCCn1c(=O)n(C2CC2OC)c(=O)c2c(C)c(Cn3nc[nH]c3=O)sc21]

Analogously to the process described in Ex. 195, 275 mg (0.553 mmol) of the compound from Ex. 453A gave 180 mg (79% of theory) of the title compound.

¹H-NMR (600 MHz, DMSO-d₆, δ/ppm): 8.91 (broad, 1H), 7.83 (s, 1H), 4.92 (s, 2H), 4.07-3.89 (m, 2H), 3.65-3.55 (m, 2H), 3.41 (td, 1H), 3.22 (2 s, 6H), 2.61 (dt, 1H), 2.42 (s, 3H), 1.30 (td, 1H), 0.90 (ddd, 1H).

LC/MS (Method 1, ESIpos): Rₜ=0.95 min, m/z=408.13 [M+H]⁺.

### Example 310

### 3-(2-Methoxycyclopropyl)-1-(2-methoxyethyl)-5-methyl-6-[(5-oxo-4,5-dihydro-1H-1,2,4-triazol-1-yl)methyl]thieno[2,3-d]pyrimidine-2,4(1H,3H)-dione (trans enantiomer 1)

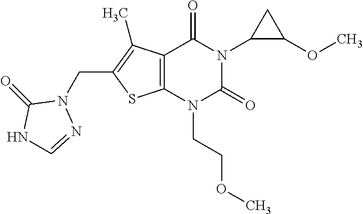 [SMILES: COCCn1c(=O)n(C2CC2OC)c(=O)c2c(C)c(Cn3nc[nH]c3=O)sc21]

170 mg of the racemic compound from Ex. 309 were dissolved in 7.5 ml of acetonitrile/ethanol (1:1) and, in 75 portions, separated into the enantiomers by preparative HPLC on a chiral phase [column: Daicel Chiralcel OX-H, 5 μm, 250 mm×20 mm; eluent: n-heptane/ethanol 30:70; flow rate: 15 ml/min; temperature: 40° C.; detection: 210 nm]. After concentration of the product fractions and drying of the solids under high vacuum, 60 mg (70% of theory) of enantiomer 1 were obtained (>99% ee, chiral analytical HPLC).

¹H-NMR (500 MHz, DMSO-d₆, δ/ppm): 11.56 (broad, 1H), 7.83 (s, 1H), 4.92 (s, 2H), 4.07-3.89 (m, 2H), 3.64-3.56 (m, 2H), 3.41 (ddd, 1H), 3.22 (2 s, 6H), 2.61 (dt, 1H), 2.42 (s, 3H), 1.30 (td, 1H), 0.90 (ddd, 1H).

Chiral analytical HPLC [column: Daicel Chiralpak OX-3, 3 μm 50 mm×4.6 mm; eluent: isohexane/ethanol 1:1; flow rate: 1 ml/min; temperature: 50° C.; detection: 220 nm]: Rₜ=4.55 min.

### Example 311

### 3-(2-Methoxycyclopropyl)-1-(2-methoxyethyl)-5-methyl-6-[(5-oxo-4,5-dihydro-H-1,2,4-triazol-1-yl)methyl]thieno[2,3-d]pyrimidine-2,4(1H,3H)-dione (trans enantiomer 2)

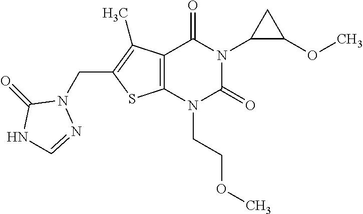 [SMILES: COCCn1c(=O)n(C2CC2OC)c(=O)c2c(C)c(Cn3nc[nH]c3=O)sc21]

48 mg (56% of theory) of enantiomer 2 were obtained from the preparative HPLC separation on a chiral phase described in Ex. 310 (>99% ee, chiral analytical HPLC). The product here, after HPLC chromatography, still as a methanolic solution, was passed through a hydrogencarbonate cartridge, then concentrated and dried under reduced pressure.

¹H-NMR (500 MHz, DMSO-d₆, δ/ppm): 7.81 (s, 1H), 4.91 (s, 2H), 4.07-3.89 (m, 2H), 3.65-3.56 (m, 2H), 3.44-3.39 (m, 1H, partially concealed by water signal), 3.22 (2 s, 6H), 2.61 (dt, 1H), 2.42 (s, 3H), 1.30 (td, 1H), 0.90 (ddd, 1H).

Chiral analytical HPLC [column: Daicel Chiralpak OX-3, 3 μm 50 mm×4.6 mm; eluent: isohexane/ethanol 1:1; flow rate: 1 ml/min; temperature: 50° C.; detection: 220 nm]: Rₜ=6.92 min.

### Example 312

### 3-Cyclobutyl-5-methyl-6-[(5-oxo-2,5-dihydro-1H-pyrazol-1-yl)methyl]-1-(3,3,3-trifluoropropyl)thieno[2,3-d]pyrimidine-2,4(1H,3H)-dione

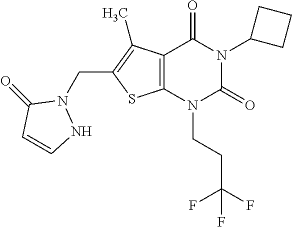 [SMILES: Cc1c(Cn2[nH]ccc2=O)sc2c1c(=O)n(C1CCC1)c(=O)n2CCC(F)(F)F]

Analogously to the method described in Ex. 200, 220 mg (0.193 mmol, 50% purity) of the compound from Ex. 457A were used to prepare 68 mg (82% of theory) of the title compound.

¹H-NMR (400 MHz, DMSO-d₆, δ/ppm): 11.34 (broad, 1H), 7.15 (s, 1H), 5.30 (s, 1H), 5.20 (quin, 1H), 5.14 (s, 2H), 4.03 (t, 2H), 2.89-2.64 (m, 4H), 2.46 (s, 3H), 2.22-2.08 (m, 2H), 1.88-1.62 (m, 2H).

LC/MS (Method 2, ESIneg): Rₜ=0.91 min, m/z=427 [M−H].

### Example 313

### 3-(3,3-Difluorocyclobutyl)-5-methyl-6-[(5-oxo-2,5-dihydro-1H-pyrazol-1-yl)methyl]-1-(3,3,3-trifluoropropyl)thieno[2,3-d]pyrimidine-2,4(1H,3H)-dione

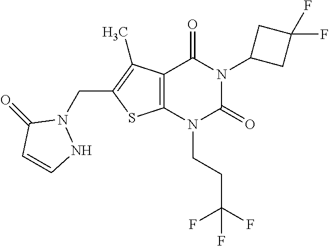 [SMILES: Cc1c(Cn2[nH]ccc2=O)sc2c1c(=O)n(C1CC(F)(F)C1)c(=O)n2CCC(F)(F)F]

Analogously to the method described in Ex. 200, 316 mg (0.518 mmol) of the compound from Ex. 458A were used to prepare 160 mg (66% of theory) of the title compound.

¹H-NMR (400 MHz, DMSO-d₆, δ/ppm): 11.29 (broad, 1H), 7.15 (s, 1H), 5.30 (s, 1H), 5.20-5.06 (m, 1H), 5.15 (s, 2H), 4.04 (t, 2H), 3.53-3.37 (m, 2H), 2.93-2.78 (m, 2H), 2.78-2.65 (m, 2H), 2.47 (s, 3H).

LC/MS (Method 1, ESIneg): Rₜ=1.68 min, m/z=463.09 [M−H].

### Example 314

### 3-(3,3-Dimethylcyclobutyl)-5-methyl-6-[(5-oxo-2,5-dihydro-H-pyrazol-1-yl)methyl]-1-(3,3,3-trifluoropropyl)thieno[2,3-d]pyrimidine-2,4(1H,3H)-dione

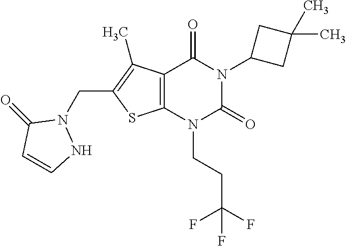 [SMILES: Cc1c(Cn2[nH]ccc2=O)sc2c1c(=O)n(C1CC(C)(C)C1)c(=O)n2CCC(F)(F)F]

Analogously to the method described in Ex. 200, 270 mg (0.448 mmol) of the compound from Ex. 459A were used to prepare 123 mg (60% of theory) of the title compound. In this case, the reaction time was only 5 min.

¹H-NMR (400 MHz, DMSO-d₆, δ/ppm): 11.16 (br. s, 1H), 7.15 (br. s, 1H), 5.31 (d, 1H), 5.20 (quin, 1H), 5.15 (s, 2H), 4.03 (t, 2H), 2.80-2.61 (m, 4H), 2.46 (s, 3H), 2.01-1.89 (m, 2H), 1.18 (s, 6H).

LC/MS (Method 2, ESIneg): Rₜ=1.03 min, m/z=455 [M−H].

### Example 315

### 1-(2-Methoxyethyl)-5-methyl-3-[(1S,2S)-2-methylcyclopropyl]-6-[(2-oxoimidazolidin-1-yl)methyl]thieno[2,3-d]pyrimidine-2,4(1H,3H)-dione

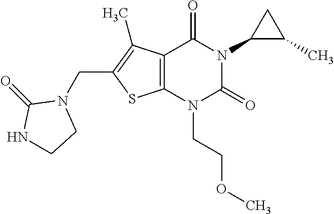 [SMILES: COCCn1c(=O)n([C@H]2C[C@@H]2C)c(=O)c2c(C)c(CN3CCNC3=O)sc21]

To a suspension of 273 g (621 mmol) of the compound from Ex. 377A in 5.7 litres of THF were added 303 ml (2.17 mol) of triethylamine and 151 g (932 mmol) of CDI. The reaction mixture was stirred at RT for 55 h. Then the volatile constituents were removed on a rotary evaporator. The residue that remained was taken up in 12 litres of ethyl acetate and washed twice with 4 litres each time of 2 M hydrochloric acid. The combined aqueous phases were extracted twice with 4 litres each time of ethyl acetate. All ethyl acetate phases were combined and washed successively with 4 litres each time of aqueous sodium chloride solution (10%) and aqueous sodium hydrogencarbonate solution (10%). The mixture was dried over anhydrous sodium sulfate, filtered and concentrated. The solids that remained were admixed with a little tert-butyl methyl ether and stirred at RT for 1 h. Then the solids were filtered off with suction and dried at 50° C. under reduced pressure. 167 g (68% of theory) of the title compound were obtained, which is identical to the compound from Ex. 96.

¹H-NMR (400 MHz, DMSO-d₆, δ/ppm): 6.51 (s, 1H), 4.33 (s, 2H), 4.05-3.90 (m, 2H), 3.60 (t, 2H), 3.28-3.15 (m, 4H), 3.23 (s, 3H), 2.37 (s, 3H), 2.27-2.20 (m, 1H), 1.15 (d, 3H), 1.05-0.92 (m, 1H), 0.87-0.78 (m, 2H).

LC/MS (Method 6, ESIpos): Rₜ=0.99 min, m/z=393 [M+H]⁺.

Crystallization: 6.75 g of the title compound were heated to reflux in a mixture of 135 ml of water and 15 ml of ethanol, in the course of which the solid just went completely into solution. The mixture was hot-filtered through a fluted filter. The filtrate was heated to reflux again for 30 min. Then the heating bath was turned down to 80° C. and the mixture was stirred at this temperature for 3 h. During this period, a portion of the product gradually crystallized out. Then the heating bath was turned back up to 90° C. and the suspension was stirred at this temperature for 1 h. Subsequently, the temperature of the heating bath was reduced stepwise, and stirring was effected over the following periods of time at the specified temperatures: 15 h 80° C.→75 min 70° C.→75 min 60° C.→75 min 50° C.-75 min 40° C.→60 min 30° C.→60 min RT. Thereafter, the product was filtered off with suction, washed with 15 ml of water/ethanol (9:1), and dried under high vacuum at RT. 5.83 g (86% of theory) of the title compound were obtained in crystalline form.

¹H-NMR (400 MHz, DMSO-d₆, δ/ppm): 6.51 (s, 1H), 4.33 (s, 2H), 4.05-3.89 (m, 2H), 3.60 (t, 2H), 3.28-3.15 (m, 4H), 3.23 (s, 3H), 2.37 (s, 3H), 2.27-2.20 (m, 1H), 1.15 (d, 3H), 1.05-0.93 (m, 1H), 0.87-0.78 (m, 2H).

LC/MS (Method 2, ESIpos): Rₜ=0.73 min, m/z=393 [M+H]⁺.

Chiral analytical HPLC [column: Daicel Chiralpak AY-3, 3 μm 50 mm×4.6 mm; eluent: isohexane/ethanol 1:1; flow rate: 1 ml/min; temperature: RT; detection: 220 nm]: Rₜ=2.13 min, ee=99.5%.

Specific optical rotation: [α]_{D}²⁰=+46.7°·ml·dm⁻¹·g⁻¹ (Chloroform).

Melting point: 167° C.

### Example 316

### 1-Ethyl-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

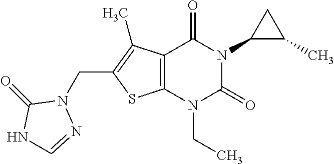 [SMILES: CCn1c(=O)n([C@H]2C[C@@H]2C)c(=O)c2c(C)c(Cn3nc[nH]c3=O)sc21]

Analogously to the method described in Ex. 220, 287 mg (0.553 mmol, 87% purity) of the compound from Ex. 515A and 10 ml of trimethyl orthoformate were used to prepare 141 mg (70% of theory) of the title compound.

¹H-NMR (500 MHz, DMSO-d₆, δ/ppm): 11.59 (br. s, 1H), 7.83 (s, 1H), 4.92 (s, 2H), 3.91-3.78 (m, 2H), 2.44 (s, 3H), 2.26-2.19 (m, 1H), 1.20 (t, 3H), 1.14 (d, 3H), 1.03-0.92 (m, 1H), 0.87-0.77 (m, 2H).

LC/MS (Method 1, ESIpos): Rₜ=1.23 min, m/z=362.13 [M+H]⁺.

### Example 317

### 5-Methyl-3-[(1S,2S)-2-methylcyclopropyl]-6-[(5-oxo-4,5-dihydro-1H-1,2,4-triazol-1-yl)methyl]-1-propylthieno[2,3-d]pyrimidine-2,4(1H,3H)-dione

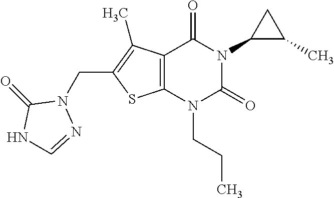 [SMILES: CCCn1c(=O)n([C@H]2C[C@@H]2C)c(=O)c2c(C)c(Cn3nc[nH]c3=O)sc21]

Analogously to the method described in Ex. 220, 323 mg (0.611 mmol, 88% purity) of the compound from Ex. 516A and 14 ml of trimethyl orthoformate were used to prepare 190 mg (82% of theory) of the title compound.

¹H-NMR (500 MHz, DMSO-d₆, δ/ppm): 11.59 (br. s, 1H), 7.82 (s, 1H), 4.92 (s, 2H), 3.84-3.69 (m, 2H), 2.44 (s, 3H), 2.26-2.19 (m, 1H), 1.66 (sext, 2H), 1.14 (d, 3H), 1.02-0.93 (m, 1H), 0.89 (t, 3H), 0.84-0.78 (m, 2H).

LC/MS (Method 1, ESIpos): Rₜ=1.38 min, m/z=376.14 [M+H]⁺.

### Example 318

### 1-Butyl-5-methyl-3-[(1S,2S)-2-methylcyclopropyl]-6-[(5-oxo-4,5-dihydro-H-1,2,4-triazol-1-yl)methyl]thieno[2,3-d]pyrimidine-2,4(1H,3H)-dione

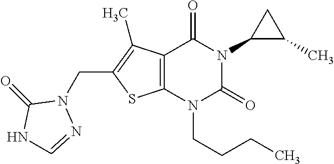 [SMILES: CCCCn1c(=O)n([C@H]2C[C@@H]2C)c(=O)c2c(C)c(Cn3nc[nH]c3=O)sc21]

Analogously to the method described in Ex. 220, 386 mg (0.757 mmol, 94% purity) of the compound from Ex. 517A and 15 ml of trimethyl orthoformate were used to prepare 199 mg (67% of theory) of the title compound.

¹H-NMR (500 MHz, DMSO-d₆, δ/ppm): 11.58 (br. s, 1H), 7.83 (s, 1H), 4.92 (s, 2H), 3.87-3.74 (m, 2H), 2.43 (s, 3H), 2.26-2.19 (m, 1H), 1.61 (quin, 2H), 1.32 (sext, 2H), 1.14 (d, 3H), 1.02-0.93 (m, 1H), 0.90 (t, 3H), 0.81 (dd, 2H).

LC/MS (Method 2, ESIpos): Rₜ=0.85 min, m/z=390 [M+H]⁺.

### Example 319

### 1-(2-Fluoroethyl)-5-methyl-3-[(1S,2S)-2-methylcyclopropyl]-6-[(5-oxo-4,5-dihydro-H-1,2,4-triazol-1-yl)methyl]thieno[2,3-d]pyrimidine-2,4(1H,3H)-dione

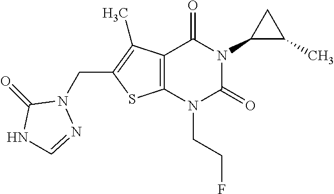 [SMILES: Cc1c(Cn2nc[nH]c2=O)sc2c1c(=O)n([C@H]1C[C@@H]1C)c(=O)n2CCF]

Analogously to the method described in Ex. 220, 465 mg (0.792 mmol, 80% purity) of the compound from Ex. 518A and 20 ml of trimethyl orthoformate were used to prepare 137 mg (45% of theory) of the title compound. The product here, after the preparative HPLC, was additionally purified by stirring in acetonitrile.

¹H-NMR (500 MHz, DMSO-d₆, δ/ppm): 11.61 (br. s, 1H), 7.83 (s, 1H), 4.92 (s, 2H), 4.70 (dt, 2H), 4.22-4.04 (m, 2H), 2.44 (s, 3H), 2.24 (dt, 1H), 1.15 (d, 3H), 1.05-0.95 (m, 1H), 0.89-0.79 (m, 2H).

LC/MS (Method 1, ESIpos): Rₜ=1.21 min, m/z=380.12 [M+H]⁺.

### Example 320

### 1-(2,2-Difluoroethyl)-5-methyl-3-[(S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

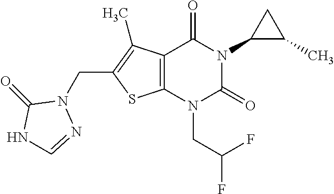 [SMILES: Cc1c(Cn2nc[nH]c2=O)sc2c1c(=O)n([C@H]1C[C@@H]1C)c(=O)n2CC(F)F]

Analogously to the method described in Ex. 220, 275 mg (0.367 mmol, 65% purity) of the compound from Ex. 519A and 10 ml of trimethyl orthoformate were used to prepare 76 mg (52% of theory) of the title compound.

¹H-NMR (500 MHz, DMSO-d₆, δ/ppm): 11.60 (br. s, 1H), 7.83 (s, 1H), 6.31 (tt, 1H), 4.93 (s, 2H), 4.36-4.16 (m, 2H), 2.44 (s, 3H), 2.29-2.22 (m, 1H), 1.15 (d, 3H), 1.06-0.95 (m, 1H), 0.90-0.79 (m, 2H).

LC/MS (Method 1, ESIpos): Rₜ=1.31 min, m/z=398.11 [M+H]⁺.

### Example 321

### 5-Methyl-3-[(1S,2S)-2-methylcyclopropyl]-6-[(5-oxo-4,5-dihydro-1H-1,2,4-triazol-1-yl)methyl]-1-(2,2,2-trifluoroethyl)thieno[2,3-d]pyrimidine-2,4(1H, 3H)-dione

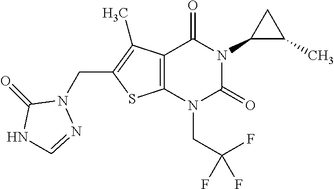 [SMILES: Cc1c(Cn2nc[nH]c2=O)sc2c1c(=O)n([C@H]1C[C@@H]1C)c(=O)n2CC(F)(F)F]

Analogously to the method described in Ex. 220, 250 mg (0.347 mmol, 70% purity) of the compound from Ex. 520A and 10 ml of trimethyl orthoformate were used to prepare 55 mg (38% of theory) of the title compound.

¹H-NMR (500 MHz, DMSO-d₆, δ/ppm): 11.61 (br. s, 1H), 7.84 (s, 1H), 4.95 (s, 2H), 4.85-4.68 (m, 2H), 2.45 (s, 3H), 2.31-2.25 (m, 1H), 1.15 (d, 3H), 1.04-0.95 (m, 1H), 0.88-0.80 (m, 2H).

LC/MS (Method 1, ESIpos): Rₜ=1.44 min, m/z=416.10 [M+H]⁺.

### Example 322

### 1-(3-Fluoropropyl)-5-methyl-3-[(1S,2S)-2-methylcyclopropyl]-6-[(5-oxo-4,5-dihydro-H-1,2,4-triazol-1-yl)methyl]thieno[2,3-d]pyrimidine-2,4(1H,3H)-dione

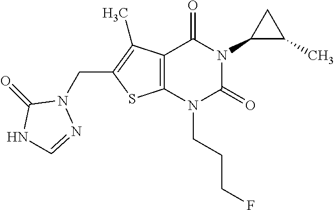 [SMILES: Cc1c(Cn2nc[nH]c2=O)sc2c1c(=O)n([C@H]1C[C@@H]1C)c(=O)n2CCCF]

Analogously to the method described in Ex. 220, 285 mg (0.589 mmol) of the compound from Ex. 521A and 12 ml of trimethyl orthoformate were used to prepare 182 mg (78% of theory) of the title compound.

¹H-NMR (500 MHz, DMSO-d₆, δ/ppm): 11.60 (br. s, 1H), 7.83 (s, 1H), 4.93 (s, 2H), 4.52 (dt, 2H), 4.01-3.85 (m, 2H), 2.44 (s, 3H), 2.27-2.19 (m, 1H), 2.11-1.94 (m, 2H), 1.15 (d, 3H), 1.04-0.93 (m, 1H), 0.87-0.77 (m, 2H).

LC/MS (Method 1, ESIpos): Rₜ=1.31 min, m/z=394.13 [M+H]⁺.

### Example 323

### 5-Methyl-3-[(1S,2S)-2-methylcyclopropyl]-6-[(5-oxo-4,5-dihydro-1H-1,2,4-triazol-1-yl)methyl]-1-(3,3,3-trifluoropropyl)thieno[2,3-d]pyrimidine-2,4(1H,3H)-dione

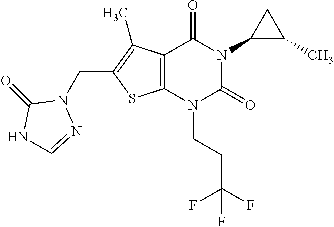 [SMILES: Cc1c(Cn2nc[nH]c2=O)sc2c1c(=O)n([C@H]1C[C@@H]1C)c(=O)n2CCC(F)(F)F]

Analogously to the method described in Ex. 220, 316 mg (0.572 mmol, 94% purity) of the compound from Ex. 522A and 12 ml of trimethyl orthoformate were used to prepare 172 mg (70% of theory) of the title compound.

¹H-NMR (500 MHz, DMSO-d₆, δ/ppm): 11.59 (br. s, 1H), 7.83 (s, 1H), 4.94 (s, 2H), 4.13-3.94 (m, 2H), 2.72 (qt, 2H), 2.44 (s, 3H), 2.27-2.20 (m, 1H), 1.15 (d, 3H), 1.03-0.93 (m, 1H), 0.87-0.77 (m, 2H).

LC/MS (Method 2, ESIpos): Rₜ=0.82 min, m/z=430 [M+H]⁺.

### Example 324

### 5-Methyl-3-[(1S,2S)-2-methylcyclopropyl]-6-[(5-oxo-4,5-dihydro-1H-1,2,4-triazol-1-yl)methyl]-1-(4,4,4-trifluorobutyl)thieno[2,3-d]pyrimidine-2,4(1H, 3H)-dione

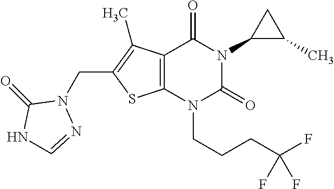 [SMILES: Cc1c(Cn2nc[nH]c2=O)sc2c1c(=O)n([C@H]1C[C@@H]1C)c(=O)n2CCCC(F)(F)F]

Analogously to the method described in Ex. 220, 310 mg (0.523 mmol, 90% purity) of the compound from Ex. 523A and 13 ml of trimethyl orthoformate were used to prepare 196 mg (84% of theory) of the title compound.

¹H-NMR (500 MHz, DMSO-d₆, δ/ppm): 11.58 (s, 1H), 7.83 (d, 1H), 4.93 (s, 2H), 3.97-3.82 (m, 2H), 2.47-2.33 (m, 2H), 2.44 (s, 3H), 2.25-2.18 (m, 1H), 1.86 (quin, 2H), 1.14 (d, 3H), 1.03-0.93 (m, 1H), 0.87-0.76 (m, 2H).

LC/MS (Method 1, ESIpos): Rₜ=1.58 min, m/z=444.13 [M+H]⁺.

### Example 325

### 1-(2-Methoxyethyl)-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

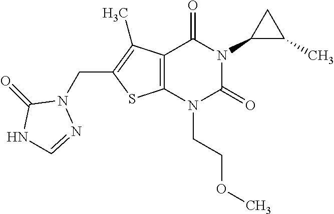 [SMILES: COCCn1c(=O)n([C@H]2C[C@@H]2C)c(=O)c2c(C)c(Cn3nc[nH]c3=O)sc21]

Analogously to the method described in Ex. 220, 243 mg (0.492 mmol, 97% purity) of the compound from Ex. 524A and 12 ml of trimethyl orthoformate were used to prepare 157 mg (81% of theory) of the title compound.

¹H-NMR (500 MHz, DMSO-d₆, δ/ppm): 11.58 (br. s, 1H), 7.83 (s, 1H), 4.91 (s, 2H), 4.02-3.89 (m, 2H), 3.59 (t, 2H), 3.22 (s, 3H), 2.43 (s, 3H), 2.23 (dt, 1H), 1.14 (d, 3H), 1.04-0.92 (m, 1H), 0.86-0.78 (m, 2H).

LC/MS (Method 1, ESIpos): Rₜ=1.20 min, m/z=392.14 [M+H]⁺.

### Example 326

### 1-[(2,2-Difluorocyclopropyl)methyl]-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione (diastereomer mixture)

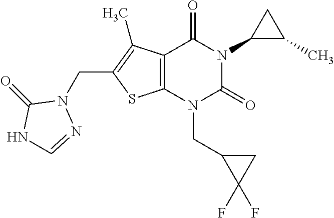 [SMILES: Cc1c(Cn2nc[nH]c2=O)sc2c1c(=O)n([C@H]1C[C@@H]1C)c(=O)n2CC1CC1(F)F]

Analogously to the method described in Ex. 220, 385 mg (0.630 mmol, 84% purity) of the compound from Ex. 525A and 15 ml of trimethyl orthoformate were used to prepare 188 mg (70% of theory) of the title compound.

¹H-NMR (500 MHz, DMSO-d₆, δ/ppm): 11.59 (br. s, 1H), 7.83 (s, 1H), 4.93 (s, 2H), 4.07 (tdd, 1H), 3.94-3.80 (m, 1H), 2.44 (s, 3H), 2.27-2.21 (m, 1H), 2.21-2.09 (m, 1H), 1.74-1.62 (m, 1H), 1.49-1.38 (m, 1H), 1.15 (d, 3H), 1.04-0.94 (m, 1H), 0.88-0.77 (m, 2H).

LC/MS (Method 1, ESIpos): Rₜ=1.45 min, m/z=424.12 [M+H]⁺.

### Example 327

### 1-(Cyclobutylmethyl)-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

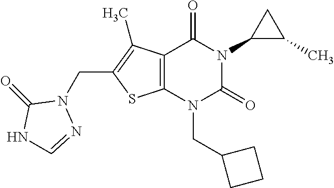 [SMILES: Cc1c(Cn2nc[nH]c2=O)sc2c1c(=O)n([C@H]1C[C@@H]1C)c(=O)n2CC1CCC1]

Analogously to the method described in Ex. 220, 270 mg (0.494 mmol, 90% purity) of the compound from Ex. 526A and 12 ml of trimethyl orthoformate were used to prepare 152 mg (76% of theory) of the title compound.

¹H-NMR (500 MHz, DMSO-d₆, δ/ppm): 11.59 (s, 1H), 7.83 (d, 1H), 4.91 (s, 2H), 3.95-3.79 (m, 2H), 2.78-2.67 (m, 1H), 2.43 (s, 3H), 2.27-2.19 (m, 1H), 2.03-1.88 (m, 2H), 1.86-1.73 (m, 4H), 1.14 (d, 3H), 1.01-0.91 (m, 1H), 0.86-0.75 (m, 2H).

LC/MS (Method 1, ESIpos): Rₜ=1.61 min, m/z=402.16 [M+H]⁺.

### Example 328

### 1-[(3,3-Difluorocyclobutyl)methyl]-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione

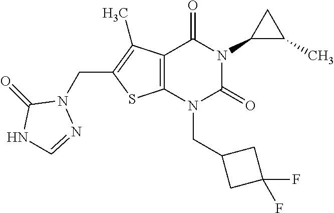 [SMILES: Cc1c(Cn2nc[nH]c2=O)sc2c1c(=O)n([C@H]1C[C@@H]1C)c(=O)n2CC1CC(F)(F)C1]

285 mg (0.540 mmol) of the compound from Ex. 527A were dissolved in a mixture of 12.5 ml each of methanol and trimethyl orthoformate, and 2 ml (8.10 mmol) of a 4 M solution of hydrogen chloride in dioxane were added at RT. After the reaction mixture had been stirred at RT for about 16 h, it was concentrated to dryness and then purified by means of preparative HPLC (Method 13). After concentration of the product fraction and drying under high vacuum, 172 mg (73% of theory) of the title compound were obtained.

¹H-NMR (500 MHz, DMSO-d₆, δ/ppm): 11.59 (br. s, 1H), 7.83 (s, 1H), 4.92 (s, 2H), 4.08-3.90 (m, 2H), 2.73-2.62 (m, 2H), 2.61-2.54 (m, 1H), 2.53-2.45 (m, 2H, partially concealed by DMSO signal), 2.43 (s, 3H), 2.26-2.19 (m, 1H), 1.14 (d, 3H), 1.02-0.92 (m, 1H), 0.85-0.77 (m, 2H).

LC/MS (Method 1, ESIpos): Rₜ=1.52 min, m/z=438.14 [M+H]⁺.

### Example 329

### 5-Methyl-3-[(1S,2S)-2-methylcyclopropyl]-6-[(5-oxo-4,5-dihydro-1H-1,2,4-triazol-1-yl)methyl]-1-[(2R)-tetrahydrofuran-2-ylmethyl]thieno[2,3-d]pyrimidine-2,4(1H,3H)-dione

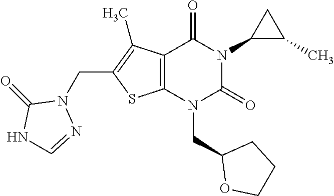 [SMILES: Cc1c(Cn2nc[nH]c2=O)sc2c1c(=O)n([C@H]1C[C@@H]1C)c(=O)n2C[C@H]1CCCO1]

Analogously to the method described in Ex. 220, 172 mg (0.220 mmol, 65% purity) of the compound from Ex. 528A and 10 ml of trimethyl orthoformate were used to prepare 36 mg (39% of theory) of the title compound.

¹H-NMR (500 MHz, DMSO-d₆, δ/ppm): 11.59 (br. s, 1H), 7.83 (s, 1H), 4.91 (s, 2H), 4.23-4.12 (m, 1H), 4.02 (dd, 1H), 3.78-3.69 (m, 1H), 3.67-3.56 (m, 2H), 2.43 (s, 3H), 2.28-2.20 (m, 1H), 2.02-1.92 (m, 1H), 1.92-1.75 (m, 2H), 1.64 (ddt, 1H), 1.15 (d, 3H), 1.04-0.92 (m, 1H), 0.87-0.78 (m, 2H).

LC/MS (Method 1, ESIpos): Rₜ=1.29 min, m/z=418.15 [M+H]⁺.

### Example 330

### 1-[(2,2-Difluorocyclopropyl)methyl]-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione (diastereomer 1)

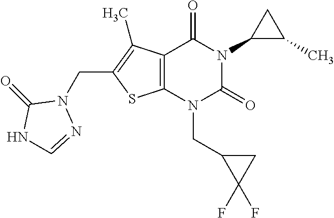 [SMILES: Cc1c(Cn2nc[nH]c2=O)sc2c1c(=O)n([C@H]1C[C@@H]1C)c(=O)n2CC1CC1(F)F]

179 mg of the diastereomer mixture from Ex. 326 were dissolved in a mixture of 5 ml of acetonitrile and 2 ml of ethanol and, in 47 portions, separated into the diastereomers via preparative HPLC on a chiral phase [column: Daicel Chiralpak AS-H, 5 μm, 250 mm×20 mm; eluent: n-heptane/ethanol 20:80; flow rate: 15 ml/min; temperature: 50° C.; detection: 210 nm]. After concentration of the product fractions and drying of the solids under high vacuum, 88 mg (98% of theory) of diastereomer 1 were obtained (>99% de, chiral analytical HPLC).

¹H-NMR (500 MHz, DMSO-d₆, δ/ppm): 11.58 (br. s, 1H), 7.83 (s, 1H), 4.93 (s, 2H), 4.06 (ddd, 1H), 3.88 (dd, 1H), 2.44 (s, 3H), 2.28-2.22 (m, 1H), 2.21-2.10 (m, 1H), 1.74-1.63 (m, 1H), 1.49-1.38 (m, 1H), 1.15 (d, 3H), 1.03-0.94 (m, 1H), 0.88-0.77 (m, 2H).

Chiral analytical HPLC [column: Daicel Chiralpak AS-3, 3 μm, 50 mm×4.6 mm; eluent: ethanol; flow rate: 1 ml/min; temperature: 50° C.; detection: 220 nm]: Rₜ=1.12 min.

### Example 331

### 1-[(2,2-Difluorocyclopropyl)methyl]-5-methyl-3-[(1S,2S)-2-methylcyclopropyl]-6-[(5-oxo-4,5-dihydro-1H-1,2,4-triazol-1-yl)methyl]thieno[2,3-d]pyrimidine-2,4(1H,3H)-dione (diastereomer 2)

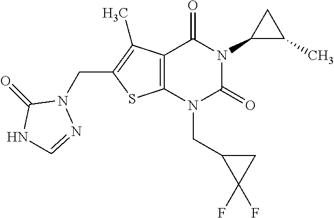 [SMILES: Cc1c(Cn2nc[nH]c2=O)sc2c1c(=O)n([C@H]1C[C@@H]1C)c(=O)n2CC1CC1(F)F]

83 mg (92% of theory) of diastereomer 2 were obtained from the diastereomer separation described in Ex. 330 (99% de, chiral analytical HPLC).

¹H-NMR (500 MHz, DMSO-d₆, δ/ppm): 11.56 (br. s, 1H), 7.83 (s, 1H), 4.93 (s, 2H), 4.09 (ddd, 1H), 3.86 (dd, 1H), 2.44 (s, 3H), 2.27-2.22 (m, 1H), 2.21-2.10 (m, 1H), 1.73-1.62 (m, 1H), 1.49-1.39 (m, 1H), 1.15 (d, 3H), 1.04-0.94 (m, 1H), 0.87-0.78 (m, 2H).

Chiral analytical HPLC [column: Daicel Chiralpak AS-3, 3 μm, 50 mm×4.6 mm; eluent: ethanol; flow rate: 1 ml/min; temperature: 50° C.; detection: 220 nm]: Rₜ=2.70 min.

### B. ASSESSMENT OF PHARMACOLOGICAL EFFICACY

The pharmacological activity of the compounds of the invention can be demonstrated by in vitro and in vivo studies as known to the person skilled in the art. The application examples which follow describe the biological action of the compounds of the invention, without restricting the invention to these examples.

### B-1. Cellular In Vitro Tests for Determining A2b Receptor Activity and Adenosine Receptor Selectivity

The identification of selective antagonists of the human adenosine A2b receptor and the quantification of the efficacy and selectivity of the compounds according to the invention was carried out with the aid of recombinant cell lines for the human adenosine receptors A1, A2a, A2b and A3. These cell lines were originally derived from an ovarepithelial cell of the hamster (Chinese Hamster Ovary, CHO-K1, American Type Culture Collection, Manassas, Va. 20108, USA). In addition to the respective recombinantly expressed adenosine receptor for testing the efficacy at the A1, A2a and A2b receptors, the cell lines contain a reporter gene construct where expression of the firefly (Photinus pyralis) luciferase is under the control of a promoter which can be activated via intracellular signal cascades by stimulation of the receptors with the (not subtype-selective) adenosine receptor agonist NECA (5′-N-ethylcarboxamidoadenosine) [S. J. Hill, J. G. Baker, S. Rhees, Curr. Opin. Pharmacol. 1, 526-532 (2001)].

In the case of the A2a and A2b cell lines, this is a minimal promoter having a plurality of cAMP-responsive elements (CRE). Stimulation of the Gₛ-coupled A2b or A2a receptors by NECA ultimately leads, via formation of cAMP, to CRE-dependent induction of luciferase expression, which is detected 3 hours after the start of the incubation with NECA using a detection solution in a suitable luminometer. For testing the antagonists, initially, in a pre-experiment, the concentration of NECA which, at the test day in question, results in half-maximum stimulation of luciferase expression (EC50 concentration) is determined. By joint incubation of this EC₅₀ concentration of NECA with the substances to be tested, it is possible to determine their antagonistic activity.

The cell line for testing the Gᵢ-coupled A1 receptor contains a different reporter gene construct where expression of the firefly luciferase is under the control of an NFAT (nuclear factor of activated T-cells) promoter. This cell line was, in addition to the A1 receptor and the NFAT reporter gene, also stably transfected with a further gene coding for the promiscuous Gα₁₆ protein [T. T. Amatruda, D. A. Steele, V. Z. Slepak, M. I. Simon, Proc. Natl. Acad. Sci. USA 88, 5587-5591 (1991)], either independently or as a fusion gene. The resulting test cells react to stimulation of the usually Gᵢ-coupled A1 receptor with an increased intracellular calcium concentration which then leads to a NFAT-dependent luciferase expression. The procedure of the experiment for testing the antagonists at the A1 receptor corresponds to the procedure for testing with the A2a and A2b cell lines.

During generation of the A3 receptor cell line, co-transfection of the A3 receptor and the promiscuous Gα₁₆ protein were also carried out so that here, too, stimulation of the receptor leads to an increased intracellular calcium concentration. However, in the A3 receptor test, this increase in calcium is measured directly via the calcium-sensitive photoprotein Photina® [S. Bovolenta, M. Foti, S. Lohmer, S. Corazza, J. Biomol. Screen. 12, 694-704 (2007)]. After determination of the EC₅₀ concentration of NECA, the effects of the substance were measured after 5-10 minutes of pre-incubation with substance by addition of this EC₅₀ concentration in measuring position in a suitable luminometer capable of dispensing.

Table 1 below lists the IC₅₀ values from the A2b receptor assay for individual working examples (in some cases as means of a plurality of independent individual determinations and rounded to two significant figures; it may also be the case here that various independent preparations of the respective working example have been used):

TABLE 1

| Example | A2b receptor |
|---|---|
| No. | IC₅₀ [nmol/l] |
| 1 | 20 |
| 2 | 81 |
| 3 | 6.9 |
| 4 | 34 |
| 5 | 12 |
| 6 | 12 |
| 7 | 18 |
| 8 | 44 |
| 9 | 1.5 |
| 10 | 1.5 |
| 11 | 3.3 |
| 12 | 4.2 |
| 13 | 4.0 |
| 14 | 3.7 |
| 15 | 14 |
| 16 | 3.0 |
| 17 | 4.1 |
| 18 | 25 |
| 19 | 19 |
| 20 | 130 |
| 21 | 53 |
| 22 | 280 |
| 23 | 130 |
| 24 | 29 |
| 25 | 12 |
| 26 | 8.3 |
| 27 | 40 |
| 28 | 19 |
| 29 | 57 |
| 30 | 9.3 |
| 31 | 67 |
| 32 | 430 |
| 33 | 100 |
| 34 | 500 |
| 35 | 1.8 |
| 36 | 4.4 |
| 37 | 35 |
| 38 | 33 |
| 39 | 240 |
| 40 | 15 |
| 41 | 16 |
| 42 | 10 |
| 43 | 12 |
| 44 | 16 |
| 45 | 110 |
| 46 | 39 |
| 47 | 140 |
| 48 | 56 |
| 49 | 110 |
| 50 | 140 |
| 51 | 320 |
| 52 | 19 |
| 53 | 120 |
| 54 | 7.5 |
| 55 | 49 |
| 56 | 3.6 |
| 57 | 4.4 |
| 58 | 10 |
| 59 | 9.3 |
| 60 | 15 |
| 61 | 5.4 |
| 62 | 65 |
| 63 | 12 |
| 64 | 13 |
| 65 | 36 |
| 66 | 140 |
| 67 | 54 |
| 68 | 160 |
| 69 | 120 |
| 70 | 15 |
| 71 | 12 |
| 72 | 15 |
| 73 | 29 |
| 74 | 86 |
| 75 | 3.3 |
| 76 | 8.5 |
| 77 | 77 |
| 78 | 560 |
| 79 | 110 |
| 80 | 740 |
| 81 | 39 |
| 82 | 77 |
| 83 | 130 |
| 84 | 460 |
| 85 | 310 |
| 86 | 270 |
| 87 | 140 |
| 88 | 690 |
| 89 | 1200 |
| 90 | 5.3 |
| 91 | 20 |
| 92 | 2.8 |
| 93 | 1.8 |
| 94 | 7.2 |
| 95 | 26 |
| 96 | 3.8 |
| 97 | 26 |
| 98 | 140 |
| 99 | 28 |
| 100 | 12 |
| 101 | 32 |
| 102 | 7.3 |
| 103 | 21 |
| 104 | 35 |
| 105 | 92 |
| 106 | 41 |
| 107 | 290 |
| 108 | 110 |
| 109 | 640 |
| 110 | 0.59 |
| 111 | 1.7 |
| 112 | 1.2 |
| 113 | 2.0 |
| 114 | 1.9 |
| 115 | 4.3 |
| 116 | 4.0 |
| 117 | 1.3 |
| 118 | 14 |
| 119 | 12 |
| 120 | 3.2 |
| 121 | 15 |
| 122 | 4.2 |
| 123 | 10 |
| 124 | 26 |
| 125 | 33 |
| 126 | 14 |
| 127 | 10 |
| 128 | 36 |
| 129 | 280 |
| 130 | 14 |
| 131 | 68 |
| 132 | 11 |
| 133 | 430 |
| 134 | 34 |
| 135 | 48 |
| 136 | 19 |
| 137 | 77 |
| 138 | 260 |
| 139 | 310 |
| 140 | 230 |
| 141 | 110 |
| 142 | 460 |
| 143 | 880 |
| 144 | 10 |
| 145 | 23 |
| 146 | 6.2 |
| 147 | 48 |
| 148 | 66 |
| 149 | 82 |
| 150 | 120 |
| 151 | 45 |
| 152 | 340 |
| 153 | 220 |
| 154 | 23 |
| 155 | 56 |
| 156 | 18 |
| 157 | 63 |
| 158 | 180 |
| 159 | 200 |
| 160 | 200 |
| 161 | 130 |
| 162 | 730 |
| 163 | 1200 |
| 164 | 4.6 |
| 165 | 14 |
| 166 | 4.7 |
| 167 | 5.3 |
| 168 | 16 |
| 169 | 14 |
| 170 | 8.6 |
| 171 | 17 |
| 172 | 36 |
| 173 | 83 |
| 174 | 2.8 |
| 175 | 18 |
| 176 | 4.0 |
| 177 | 17 |
| 178 | 26 |
| 179 | 10 |
| 180 | 13 |
| 181 | 6.7 |
| 182 | 27 |
| 183 | 70 |
| 184 | 6.9 |
| 185 | 16 |
| 186 | 7.9 |
| 187 | 19 |
| 188 | 100 |
| 189 | 40 |
| 190 | 28 |
| 191 | 18 |
| 192 | 140 |
| 193 | 230 |
| 194 | 180 |
| 195 | 120 |
| 196 | 210 |
| 197 | 120 |
| 198 | 190 |
| 199 | 130 |
| 200 | 55 |
| 201 | 53 |
| 202 | 140 |
| 203 | 160 |
| 204 | 1.8 |
| 205 | 7.0 |
| 206 | 6.2 |
| 207 | 910 |
| 208 | 10 |
| 209 | 200 |
| 210 | 200 |
| 211 | 1300 |
| 212 | 240 |
| 213 | 40 |
| 214 | 10 |
| 215 | 73 |
| 216 | 3.2 |
| 217 | 38 |
| 218 | 54 |
| 219 | 400 |
| 220 | 33 |
| 221 | 3.3 |
| 222 | 1.6 |
| 223 | 19 |
| 224 | 11 |
| 225 | 26 |
| 226 | 1.9 |
| 227 | 2.9 |
| 228 | 5.8 |
| 229 | 1.2 |
| 230 | 1.3 |
| 231 | 2.3 |
| 232 | 0.86 |
| 233 | 11 |
| 234 | 1.9 |
| 235 | 18 |
| 236 | 1.2 |
| 237 | 4.4 |
| 238 | 24 |
| 239 | 4.7 |
| 240 | 2.4 |
| 241 | 7.1 |
| 242 | 2.1 |
| 243 | 8.4 |
| 244 | 3.3 |
| 245 | 6.7 |
| 246 | 22 |
| 247 | 3.5 |
| 248 | 1.2 |
| 249 | 1.9 |
| 250 | 1.1 |
| 251 | 6.3 |
| 252 | 1.4 |
| 253 | 2.7 |
| 254 | 7.2 |
| 255 | 6.7 |
| 256 | 5.5 |
| 257 | 6.8 |
| 258 | 6.4 |
| 259 | 4.1 |
| 260 | 82 |
| 261 | 15 |
| 262 | 1.2 |
| 263 | 1.1 |
| 264 | 16 |
| 265 | 1.3 |
| 266 | 0.96 |
| 267 | 2.4 |
| 268 | 1.5 |
| 269 | 2.0 |
| 270 | 1.5 |
| 271 | 1.4 |
| 272 | 1.0 |
| 273 | 67 |
| 274 | 86 |
| 275 | 63 |
| 276 | 2.0 |
| 277 | 3.9 |
| 278 | 1.6 |
| 279 | 1.6 |
| 280 | 3.4 |
| 281 | 2.1 |
| 282 | 4.1 |
| 283 | 25 |
| 284 | 8.6 |
| 285 | 4.1 |
| 286 | 17 |
| 287 | 2.8 |
| 288 | 47 |
| 289 | 35 |
| 290 | 270 |
| 291 | 19 |
| 292 | 150 |
| 293 | 12 |
| 294 | 20 |
| 295 | 120 |
| 296 | 16 |
| 297 | 120 |
| 298 | 1800 |
| 299 | 220 |
| 300 | 13 |
| 301 | 64 |
| 302 | 10 |
| 303 | 71 |
| 304 | 350 |
| 305 | 120 |
| 306 | 16 |
| 307 | 8.1 |
| 308 | 870 |
| 309 | 110 |
| 310 | 3900 |
| 311 | 44 |
| 312 | 15 |
| 313 | 93 |
| 314 | 49 |
| 315 | 3.8 |
| 316 | 25 |
| 317 | 19 |
| 318 | 12 |
| 320 | 13 |
| 321 | 17 |
| 322 | 14 |
| 323 | 3.1 |
| 324 | 5.5 |
| 325 | 43 |
| 326 | 17 |
| 327 | 8.5 |
| 328 | 4.9 |

### B-2. Adenosine Receptor Binding Assays

The binding properties of the test compounds on adenosine receptors were determined in binding studies with radioligands. For this purpose, membrane preparations of the human adenosine receptor subtypes were produced from cell lines having recombinant receptor expression (CHO cells for the A1 receptor, HEK293 cells for the A2a, A2b and A3 receptors). The following radioligands were used in the experiments: [³H]-DPCPX for the A1 receptor, [³H]-CGS 21680 for the A2a receptor, [³H]-CPX for the A2b receptor and [¹²⁵I]-AB-MECA for the A3 receptor. The test substances were each tested in 8 different concentrations and 2 repeat tests per concentration. The displacement of the particular radioligand by the test compound was expressed as percentage inhibition of the specific binding of the controls.

The IC₅₀ values (concentration which brings about half-maximum inhibition of the specific binding of the controls) and the Hill coefficients (nH) were determined by a non-linear regression analysis, using the competition curves obtained from the mean values of the repeat tests and conducting a curve fit according to the Hill equation:

Y=D+[A-D/1+(C/C50)^{nH}]

(Y=specific binding; A=left-hand asymptote of the curve; D=right-hand asymptote of the curve; C=substance concentration; C50=IC₅₀; nH=rise factor).

The inhibition constant (Kᵢ) was calculated by the Cheng-Prusoff equation:

Kᵢ═IC₅₀/(1+L/K_{D})

(L=concentration of the radioligand in the assay; K_{D}=receptor affinity of the radioligand for the receptor, determined with a Scatchard plot).

[Literature: A1 receptor: Townsend-Nicholson, A. und Schofield, P. R., J. Biol. Chem. 269: 2373-2376 (1994); A2a receptor: Luthin, D. R. et al., Mol. Pharmacol. 47: 307-313 (1995); A2b receptor: Stehle, J. H. et al., Mol. Endocrinol. 6: 384-393 (1992) and Linden et al., Mol. Pharmacol. 56: 705-713 (1999); A3 receptor: Salvatore, C. A. et al., Proc. Natl. Acad. Sci. U.S.A. 90: 10365-10369 (1993) and Jacobson, K. A. et al., Neuropharmacology 36: 1157-1165 (1997)].

Table 2 below lists the K₁ values thus determined from these binding assays for representative working examples (in some cases as means of a plurality of independent individual determinations and rounded to two significant figures; it may also be the case here that various independent preparations of the respective working example have been used):

TABLE 2

| | A2b | A1 | A2a | A3 |
|---|---|---|---|---|
| Example | receptor | receptor | receptor | receptor |
| No. | Kᵢ [nmol/l] | Kᵢ [nmol/l] | Kᵢ [nmol/l] | Kᵢ [nmol/l] |
| 15 | 3.6 | 8000 | 3000 | >10000 |
| 36 | 2.6 | 2600 | 950 | >50000 |
| 70 | 5.7 | >10000 | 4800 | >10000 |
| 96 | 3.7 | 3400 | 480 | 3700 |
| 315 | 3.7 | 3400 | 480 | 3700 |

### B-3. Measurement of NECA-Induced IL-6 Release by LL29 Fibroblasts

Stimulation of fibroblasts with adenosine or the adenosine analog 5′-N-ethylcarboxamidoadenosine (NECA) leads to release of the pro-inflammatory and pro-fibrotic cytokine IL-6 which can be prevented by inhibition of the A2b receptor.

Accordingly, confluent cells of the human fibroblast cell line LL29 were treated with the test substances and stimulated with NECA (10 μM). After an incubation time of 24 hours, the cell supernatant is removed and human IL-6 in the cell supernatant is determined by ELISA (Quantikine® IL6 ELISA, R&D Systems, Minneapolis, USA).

Table 3 below lists the IC₅₀ values obtained in this way for inhibition of IL-6 release for representative working examples (in some cases as means of a plurality of independent individual determinations and rounded to two significant figures; it may also be the case here that various independent preparations of the respective working example have been used):

TABLE 3

| Example | IL-6 release |
|---|---|
| No. | IC₅₀ [nmol/l] |
| 5 | 21 |
| 26 | 12 |
| 36 | 6.1 |
| 76 | 11 |
| 96 | 4.9 |
| 315 | 4.9 |

### B-4. Animal Model of Monocrotaline-Induced Pulmonary Hypertension

Monocrotaline-induced pulmonary hypertension of the rat is a widely used animal model of pulmonary hypertension. The pyrrolizidine alkaloid monocrotaline is, after subcutaneous injection, metabolized in the liver to the toxic monocrotalinepyrrole, and within a few days endothelium injury in the pulmonary circulation results, followed by remodeling of the small pulmonary arteries (mediahypertrophy, de novo muscularization). A single subcutaneous injection suffices to induce pronounced pulmonary hypertension in rats within 4 weeks [Cowan et al., Nature Med. 6, 698-702 (2000)].

Male Sprague-Dawley rats are used for the model. On day 0, the animals receive a subcutaneous injection of 60 mg of monocrotaline/kg. Treatment of the animals with the test substance (by gavage, by addition to the feed or drinking water, using an osmotic minipump, by subcutaneous or intraperitoneal injection or by inhalation) starts 14 days after the monocrotaline injection at the earliest and extends over a period of at least 14 days. At the end of the study, the animals are examined haemodynamically. For the haemodynamic measurement, the rats are initially anaesthetized with pentobarbital (60 mg/kg). The animals are then tracheotomized and artificially ventilated (frequency: 60 breaths/min; ratio inspiration to expiration: 50:50; positive end-expiratory pressure: 1 cm H₂O; tidal volume: 10 ml/kg of body weight; FIO₂: 0.5). Anaesthesia is maintained by inhalative isofluran anaesthesia. The systemic blood pressure is determined in the left carotid artery using a Millar microtip catheter. A polyethylene catheter is advanced via the right jugular vein into the right ventricle to determine the right-ventricular pressure. Following the haemodynamic measurements, the heart is removed, the ratio of right to left ventricle including septum is determined and the tissue is deep-frozen for expression analyses. The lung is likewise removed, the left half of the lung is fixed in formalin for histopathological examination and the right half of the lung is deep-frozen for expression analyses. Furthermore, plasma samples are obtained to determine biomarkers (for example proBNP) and plasma substance concentrations.

### B-5. Animal Model of SU5416/Hypoxia-Induced Pulmonary Hypertension

SU5416/hypoxia-induced pulmonary hypertension of the rat is a widely used animal model of pulmonary hypertension. By injection of the VEGF receptor antagonist SU5416 in combination with hypoxia, the effect of the reduced oxygen content may be enhanced, leading to changes in the endothelium in the form of plexiform lesions. A single subcutaneous injection, generally of 20 mg/kg, is, in combination with hypoxia, i.e. increased vascular shear forces by vasoconstriction, sufficient to induce severe pulmonary hypertension [Oka et al., Circ. Res. 100, 923-929 (2007)].

Male Sprague-Dawley rats or Dahl-Salz rats are used for the model. On day 0, the animals receive a subcutaneous injection of SU5416 and are kept in a controlled hypoxic atmosphere (10% oxygen). Corresponding control rats receive an injection of vehicle and are kept under normoxic conditions. Chronic hypoxia of at least 14 days with subsequent normoxia of at least 28 days leads to the development of pulmonary hypertension which can be demonstrated both functionally and morphologically. Treatment of the animals with the test substance (by gavage, by addition to the feed or drinking water, using an osmotic minipump, by subcutaneous or intraperitoneal injection or by inhalation) starts 14 days after the SU5416 injection and at the beginning of the animals being kept in a controlled hypoxic atmosphere at the earliest and extends over a period of at least 14-28 days.

At the end of the study, the animals are examined haemodynamically. For the haemodynamic measurement, the rats are initially anaesthetized with pentobarbital (60 mg/kg). The animals are then tracheotomized and artificially ventilated (frequency: 60 breaths/min; ratio inspiration to expiration: 50:50; positive end-expiratory pressure: 1 cm H₂O; tidal volume: 10 ml/kg of body weight; FIO₂: 0.5). Anaesthesia is maintained by inhalative isofluran anaesthesia. The systemic blood pressure is determined in the left carotid artery using a Millar microtip catheter. A polyethylene catheter is advanced via the right jugular vein into the right ventricle to determine the right-ventricular pressure. Following the haemodynamic measurements, the heart is removed, the ratio of right to left ventricle including septum is determined and the tissue is deep-frozen for expression analyses. The lung is likewise removed, the left half of the lung is fixed in formalin for histopathological examination and the right half of the lung is deep-frozen for expression analyses. Furthermore, plasma samples are obtained to determine biomarkers (for example proBNP) and plasma substance concentrations.

### B-6. Animal Model of Bleomycin-Induced Pulmonary Fibrosis

Bleomycin-induced pulmonary fibrosis in the mouse or rat is a widely used animal model of pulmonary fibrosis. Bleomycin is a glycopeptide antibiotic employed in oncology for the therapy of testicular tumors and Hodgkin- and Non-Hodgkin tumors. It is eliminated renally, has a half-life of about 3 hours and, as cytostatic, influences various phases of the division cycle [Lazo et al., Cancer Chemother. Biol. Response Modif. 15, 44-50 (1994)]. Its anti-neoplastic effect is based on an oxidatively damaging action on DNA [Hay et al., Arch. Toxicol. 65, 81-94 (1991)]. Lung tissue is at a particular risk when exposed to bleomycin since it contains only a small number of cysteine hydrolases which, in other tissues, lead to inactivation of bleomycin. Following administration of bleomycin, the animals suffer an acute respiratory distress syndrome (ARDS) with subsequent development of pulmonary fibrosis.

Administration of bleomycin may be by single or repeat intratracheal, inhalative, intravenous or intraperitoneal administration. Treatment of the animals with the test substance (by gavage, by addition to the feed or drinking water, using an osmotic minipump, by subcutaneous or intraperitoneal injection or by inhalation) starts at the day of the first bleomycin administration or therapeutically 3-14 days later and extends over a period of 2-6 weeks. At the end of the study, lung function measurements, a bronchio-alveolar lavage to determine the cell content and the pro-inflammatory and pro-fibrotic markers and a histological assessment of pulmonary fibrosis are carried out.

B-7. Animal model of DQ12 quartz-induced pulmonary fibrosis

DQ12 quartz-induced pulmonary fibrosis in the mouse or rat is a widely used animal model of pulmonary fibrosis [Shimbori et al., Exp. Lung Res. 36, 292-301 (2010)]. DQ12 quartz is quartz which is highly active owing to breaking or grinding. In mice and rats, intratracheal or inhalative administration of DQ12 quartz leads to alveolar proteinosis followed by interstitial pulmonary fibrosis. The animals receive a single or repeat intratracheal or inhalative instillation of DQ12 quartz. Treatment of the animals with the test substance (by gavage, by addition to the feed or drinking water, using an osmotic minipump, by subcutaneous or intraperitoneal injection or by inhalation) starts at the day of the first silicate instillation or therapeutically 3-14 days later and extends over a period of 3-20 weeks. At the end of the study, lung function measurements, a bronchio-alveolar lavage to determine the cell content and the pro-inflammatory and pro-fibrotic markers and a histological assessment of pulmonary fibrosis are carried out.

### B-8. Animal Model of DQ12 Quartz or FITC-Induced Pulmonary Inflammation

In the mouse and the rat, intratracheal administration of DQ12 quartz or fluorescein isothiocyanate (FITC) leads to an inflammation in the lung [Shimbori et al., Exp. Lung Res. 36, 292-301 (2010)]. At the day of the instillation of DQ12 quartz or FITC or a day later the animals are treated with the test substance for a duration of 24 h up to 7 days (by gavage, by addition to the feed or drinking water, using an osmotic minipump, by subcutaneous or intraperitoneal injection or by inhalation). At the end of the experiment, a bronchio-alveolar lavage to determine the cell content and the pro-inflammatory and pro-fibrotic markers is carried out.

### B-9. Animal Model of Ovalbumin-Induced Allergic Respiratory Pathway Inflammation and Hyperreactivity

The animal model of ovalbumin-induced allergic respiratory pathway inflammation and hyperreactivity is a widely used animal model for bronchial asthma [Rückert et al., J. Immunol. 174, 5507-5515 (2005)]. Mice are sensitized on days 0, 14 and 21 by means of an intraperitoneal injection with the ovalbumin allergen in combination with adjuvant; the negative control receives an intraperitoneal injection of NaCl in combination with adjuvant. On days 28 and 29, the animals receive an intratracheal instillation of ovalbumin.

On day 30, a hyperreactivity test is conducted in the form of an inhalative provocation with a stepwise rise in concentration of a bronchoconstrictor, for example methacholine or adenosine monophosphate. First of all, the animals are anaesthetized by means of injected anaesthetic, then orotracheally intubated or tracheotomized and connected to a lung function system by means of a tube. First of all, lung function is measured by body plethysmography prior to provocation (including parameters such as tidal volume, breathing frequency, dynamic compliance and lung resistance). This is followed by measurement of lung function on inhalative provocation with a stepwise rise in concentration of the bronchoconstrictor. Thereafter, a bronchio-alveolar lavage is conducted to determine the cell content and the pro-inflammatory markers.

### B-10. Animal Model of the Elastase-Induced Pulmonary Emphysema

The elastase-induced pulmonary emphysema in the mouse, rat or hamster is a widely used animal model of pulmonary emphysema [Sawada et al., Exp. Lung Res. 33, 277-288 (2007)]. The animals receive an orotracheal instillation of porcine pancreas elastase. The treatment of the animals starts at the day of the instillation of the porcine pancreas elastase and extends over a period of 3 weeks. At the end of the study, an alveolar morphometry is carried out.

### B-11. Animal Model of Permanent Coronary Ligature in Mouse and Rat

Mice or rats are anaesthetized with 5% isoflurane in an anaesthetization cage, intubated, connected to a ventilation pump and ventilated with 2% of isoflurane/N₂O/O₂. The body temperature is maintained at 37-38° C. by a heating mat. Temgesic® is administered as painkiller. The chest is opened laterally between the third and fourth ribs, and the heart is exposed. The coronary artery of the left ventricle (LAD) is permanently ligated with an occlusion thread passed underneath shortly below its origin (below the left atrium). The thorax is closed again, and the muscle layers and the epidermis are sutured. From the day of the operation or up to a week later the animals are treated with the test substance over a period of 4-8 weeks (by gavage, by addition of the test substance to the feed or drinking water, using an osmotic minipump, by subcutaneous or intraperitoneal injection or by inhalation). A further control included is a sham group in which only the surgical procedure, but not the LAD occlusion, was performed.

At the end of the experiment, the animals are anaesthetized again [1.5% isoflurane (mouse), 2% isoflurane (rat)/N₂O/air], and a pressure catheter is introduced via the carotid artery into the left ventricle. The heart rate, left-ventricular pressure (LVP), left-ventricular end-diastolic pressure (LVEDP), contractility (dp/dt) and relaxation rate (tau) are measured there and analyzed with the aid of the Powerlab system (AD Instruments, ADI-PWLB-4SP) and the Chart5 software (SN 425-0586). A blood sample is then taken to determine the blood levels of the substance and plasma biomarkers, and the animals are sacrificed. The heart (heart chambers, left ventricle plus septum, right ventricle), liver, lung and kidney are removed and weighed.

### B-12. Animal Model of Tumour Growth

Syngeneic tumour models in immunocompetent mice and xenogeneic tumour models in immunosuppressed mice are employed for substance assessment. For this purpose, tumour cells are cultivated in vitro and implanted subcutaneously or orthotopically. The animals are treated by oral, subcutaneous, intraperitoneal or intravenous therapy after the establishment of the tumour or starting on the day of tumour inoculation. The efficacy of the test substances is analysed in monotherapy and in combination therapy with other active pharmacological substances. During the experiment, the state of health of the animals is checked daily and the treatments are effected in accordance with animal protection regulations. The tumour area is measured with slide gauges (length L, breadth B=shorter dimension). The tumour volume is calculated by the formula (L×B²)/2. The inhibition in tumour growth is determined at the end of the study as the T/C ratio of the tumour areas or tumour weights and as the TGI value (tumour growth inhibition, calculated by the formula [1−(T/C)]×100) (T=tumour size in the treated group; C=tumour size in the untreated control group).

### B-13. Animal Model of Formation of Metastases in the Lung

Syngeneic tumour models in immunocompetent mice and xenogeneic tumour models in immunosuppressed mice are employed for substance assessment. For this purpose, tumour cells are cultivated in vitro and injected into the tail vein of the test animals. The animals are treated by oral, subcutaneous, intraperitoneal or intravenous therapy. The efficacy of the test substances is analysed in monotherapy and in combination therapy with other active pharmacological substances. During the experiment, the state of health of the animals is checked daily and the treatments are effected in accordance with animal protection regulations. After the experiment has ended, the lungs of the test animals are examined microscopically with regard to the number of tumour colonies formed.

### C. WORKING EXAMPLES OF PHARMACEUTICAL COMPOSITIONS

The compounds of the invention can be converted to pharmaceutical preparations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

Suspension for oral administration:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol; the compound of the invention is added to the suspension.

The water is added while stirring. The mixture is stirred for about 6 h until the swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. A compound of formula (I) 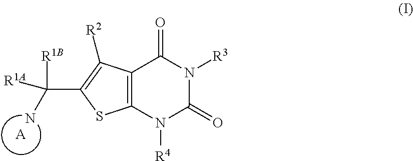
wherein
the ring A is an azaheterocycle of formula 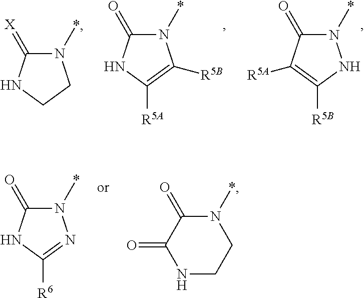
wherein * marks the bond to the adjoining C(R^{1A})(R^{1B}) group,
R^{5A} and R^{5B} are the same or different and are independently hydrogen or (C₁-C₄)-alkyl,
R⁶ is hydrogen or (C₁-C₄)-alkyl,
and
X is O, S or N(R⁷) wherein
R⁷ represents cyano, methoxycarbonyl or ethoxycarbonyl,
R^{1A} and R^{1B} are independently hydrogen or deuterium,
R² is methyl or ethyl,
R³ is cyclopropyl, cyclobutyl, cyclopentyl, spiro[3.3]hept-2-yl, 3-oxetanyl or 3-tetrahydrofuranyl,
where cyclopropyl, cyclobutyl, cyclopentyl and spiro[3.3]hept-2-yl may be up to disubstituted identically or differently by a radical selected from fluorine, methyl, ethyl, trifluoromethyl and methoxy,
and
where 3-oxetanyl and 3-tetrahydrofuranyl may be up to disubstituted identically or differently by a radical selected from fluorine and methyl,
and
R⁴ is methyl, ethyl, 2-fluoroethyl, 2,2-difluoroethyl, 2,2,2-trifluoroethyl, n-propyl, 3-cyanopropyl, 3-fluoropropyl, 3,3-difluoropropyl, 3,3,3-trifluoropropyl, n-butyl, 4-fluorobutyl, 4,4,4-trifluorobutyl, 3,3,4,4-tetrafluorobutyl, n-pentyl, iso-pentyl or n-hexyl,
or
R⁴ is a group of formula —CH₂—R⁸ wherein
R⁸ is cyano, cyclopropyl, cyclobutyl, cyclopentyl, 2-oxetanyl, 3-oxetanyl, 2-tetrahydrofuranyl or 3-tetrahydrofuranyl,
where cyclopropyl, cyclobutyl and cyclopentyl may be up to disubstituted by fluorine,
or
R⁴ is a group of formula —CH₂—CH₂—OR⁹ or —CH₂—CH₂—SR¹⁰ wherein
R⁹ is methyl, trifluoromethyl, ethyl or iso-propyl
and
R¹⁰ is methyl or trifluoromethyl,
and solvates thereof.

2. The compound of formula (I) according to claim 1 wherein
the ring A is an azaheterocycle of formula 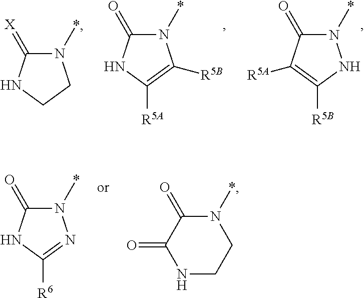
wherein * marks the bond to the adjoining C(R^{1A})(R^{1B}) group,
R^{5A} and R^{5B} are the same or different and are independently hydrogen or (C₁-C₄)-alkyl,
R⁶ is hydrogen or (C₁-C₄)-alkyl,
and
X is O, S or N(R⁷) wherein
R⁷ represents cyano, methoxycarbonyl or ethoxycarbonyl,
R^{1A} and R^{1B} are independently hydrogen or deuterium,
R² is methyl or ethyl,
R³ is cyclopropyl, cyclobutyl, cyclopentyl, spiro[3.3]hept-2-yl, 3-oxetanyl or 3-tetrahydrofuranyl,
where cyclopropyl, cyclobutyl, cyclopentyl and spiro[3.3]hept-2-yl may be up to disubstituted identically or differently by a radical selected from fluorine, methyl, ethyl, trifluoromethyl and methoxy,
and
where 3-oxetanyl and 3-tetrahydrofuranyl may be up to disubstituted identically or differently by a radical selected from fluorine and methyl,
and
R⁴ is methyl, ethyl, n-propyl, 3-fluoropropyl, 3,3-difluoropropyl, 3,3,3-trifluoropropyl, n-butyl, n-pentyl, iso-pentyl or n-hexyl,
or
R⁴ is a group of formula —CH₂—R⁸ wherein
R⁸ is cyano, cyclopropyl, cyclobutyl, cyclopentyl, 2-oxetanyl, 3-oxetanyl, 2-tetrahydrofuranyl or 3-tetrahydrofuranyl,
where cyclopropyl, cyclobutyl and cyclopentyl may be up to disubstituted by fluorine,
or
R⁴ is a group of formula —CH₂—CH₂—OR⁹ wherein
R⁹ is methyl, trifluoromethyl, ethyl or iso-propyl,
And/or a solvate thereof.

3. The compound of formula (I) according to claim 1 wherein
the ring A is an azaheterocycle of formula 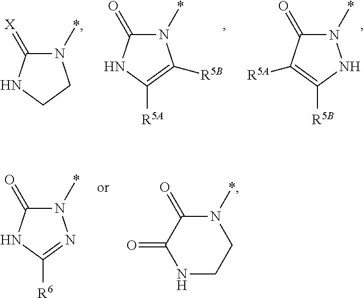
wherein * marks the bond to the adjoining C(R^{1A})(R^{1B}) group,
R^{5A} and R^{5B} are the same or different and are independently hydrogen or methyl,
R⁶ is hydrogen or methyl,
and
X is O or N(R⁷) wherein
R⁷ represents cyano or methoxycarbonyl,
R^{1A} and R^{1B} are both hydrogen or both deuterium,
R² is methyl,
R³ is cyclopropyl, cyclobutyl, cyclopentyl or spiro[3.3]hept-2-yl,
where cyclopropyl, cyclobutyl, cyclopentyl and spiro[3.3]hept-2-yl may be up to disubstituted identically or differently by a radical selected from fluorine, methyl and methoxy,
and
R⁴ is methyl, ethyl, 2-fluoroethyl, 2,2-difluoroethyl, 2,2,2-trifluoroethyl, n-propyl, 3-fluoropropyl, 3,3-difluoropropyl, 3,3,3-trifluoropropyl, n-butyl, 4,4,4-trifluorobutyl, n-pentyl or n-hexyl,
or
R⁴ is a group of formula —CH₂—R wherein
R⁸ is cyclopropyl, cyclobutyl or 2-tetrahydrofuranyl,
where cyclopropyl and cyclobutyl may be up to disubstituted by fluorine,
or
R⁴ is a group of formula —CH₂—CH₂—OR⁹ wherein
R⁹ is methyl or trifluoromethyl,
And/or a solvate thereof.

4. The compound of formula (I) according to claim 1 wherein
the ring A is an azaheterocycle of formula 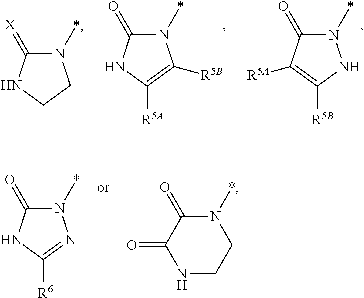
wherein * marks the bond to the adjoining C(R^{1A})(R^{1B}) group,
R^{5A} and R^{5B} are the same or different and are independently hydrogen or methyl,
R⁶ is hydrogen or methyl,
and
X is O or N(R⁷) wherein
R⁷ represents cyano or methoxycarbonyl,
R^{1A} and R^{1B} are both hydrogen or both deuterium,
R² is methyl,
R³ is cyclopropyl, cyclobutyl, cyclopentyl or spiro[3.3]hept-2-yl,
where cyclopropyl, cyclobutyl, cyclopentyl and spiro[3.3]hept-2-yl may be up to disubstituted identically or differently by a radical selected from fluorine, methyl and methoxy,
and
R⁴ is n-propyl, 3-fluoropropyl, 3,3-difluoropropyl, 3,3,3-trifluoropropyl, n-butyl, n-pentyl or n-hexyl,
or
R⁴ is a group of formula —CH₂—R⁸ wherein
R⁸ is cyclopropyl, cyclobutyl or 2-tetrahydrofuranyl,
or
R⁴ is a group of formula —CH₂—CH₂—OR⁹ wherein
R⁹ is methyl or trifluoromethyl,
And/or a solvate thereof.

5. The compound of formula (I) according to claim 1, wherein
the ring A is an azaheterocycle of formula 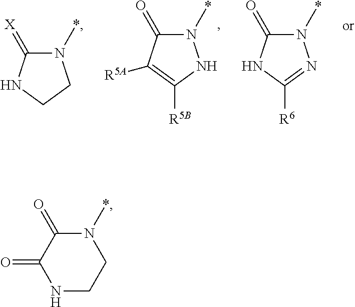
wherein * marks the bond to the adjoining C(R^{1A})(R^{1B}) group,
R^{5A} and R^{5B} are each hydrogen,
R⁶ is hydrogen,
and
X is O or N(R⁷) wherein
R⁷ represents cyano or methoxycarbonyl,
R^{1A} and R^{1B} are both hydrogen or both deuterium,
R² is methyl,
R³ is cyclopropyl, cyclobutyl or cyclopentyl,
where cyclopropyl, cyclobutyl and cyclopentyl may be up to disubstituted, identically or differently, by a radical selected from fluorine and methyl,
and
R⁴ is methyl, ethyl, 2-fluoroethyl, 2,2-difluoroethyl, 2,2,2-trifluoroethyl, n-propyl, 3-fluoropropyl, 3,3,3-trifluoropropyl or n-butyl,
or
R⁴ is a group of formula —CH₂—R⁸ wherein
R⁸ is cyclopropyl or cyclobutyl,
where cyclopropyl and cyclobutyl may be up to disubstituted by fluorine,
or
R⁴ is a group of formula —CH₂—CH₂—OR⁹ wherein
R⁹ is methyl or trifluoromethyl,
And/or a solvate thereof.

6. The compound of formula (I) according to claim 1 wherein
the ring A is an azaheterocycle of formula 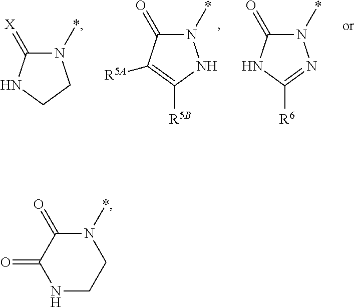
wherein * marks the bond to the adjoining C(R^{1A})(R^{1B}) group,
R^{5A} and R^{5B} are each hydrogen,
R⁶ is hydrogen,
and
X is O or N(R⁷) wherein
R⁷ represents cyano or methoxycarbonyl,
R^{1A} and R^{1B} are both hydrogen or both deuterium,
R² is methyl,
R³ is cyclopropyl, cyclobutyl or cyclopentyl,
where cyclopropyl, cyclobutyl and cyclopentyl may be up to disubstituted, identically or differently, by a radical selected from fluorine and methyl,
and
R⁴ is 3-fluoropropyl, 3,3,3-trifluoropropyl or n-butyl,
or
R⁴ is a group of formula —CH₂—R⁸ wherein
R⁸ is cyclopropyl or cyclobutyl,
or
R⁴ is a group of formula —CH₂—CH₂—OR⁹ wherein
R⁹ is methyl or trifluoromethyl,
And/or a solvate thereof.

7. The compound as defined in claim 1 for treatment and/or prevention of one or more diseases.

8. The compound as defined in claim 1 for use in a method for treatment and/or prevention of idiopathic pulmonary fibrosis, pulmonary hypertension, Bronchiolitis obliterans syndrome, chronic-obstructive pulmonary disease, asthma, cystic fibrosis, myocardial infarction, heart failure, sickle cell anaemia and/or cancer.

9. A product comprising a compound as defined in claim 1 for production of a medicament for treatment and/or prevention of idiopathic pulmonary fibrosis, pulmonary hypertension, Bronchiolitis obliterans syndrome, chronic-obstructive pulmonary disease, asthma, cystic fibrosis, myocardial infarction, heart failure, sickle cell anaemia and/or cancer.

10. Medicament comprising a compound as defined in claim 1 in combination with one or more inert, nontoxic, pharmaceutically suitable excipients.

11. Medicament comprising a compound as defined in claim 1 in combination with one or more further active ingredients selected from the group consisting of PDE 5 inhibitors, sGC activators, sGC stimulators, prostacyclin analogues, IP receptor agonists, endothelin antagonists, antifibrotic agents, antiinflammatory, immunomodulating, immunosuppressive and/or cytotoxic agents and/or compounds that inhibit the signal transduction cascade.

12. Medicament according to claim 10 for treatment and/or prevention of idiopathic pulmonary fibrosis, pulmonary hypertension, Bronchiolitis obliterans syndrome, chronic-obstructive pulmonary disease, asthma, cystic fibrosis, myocardial infarction, heart failure, sickle cell anaemia and/or cancer.

13. Method for treatment and/or prevention of idiopathic pulmonary fibrosis, pulmonary hypertension, Bronchiolitis obliterans syndrome, chronic-obstructive pulmonary disease, asthma, cystic fibrosis, myocardial infarction, heart failure, sickle cell anaemia and/or cancer in humans and animals comprising administering an effective amount of at least one compound as defined in claim 1, or a medicament thereof.
